(19) United States
(12) Patent Application Publication
(10) Pub. No.: US 2026/0132404 A1
(43) Pub. Date: May 14, 2026

(54) MODIFIED OLIGONUCLEOTIDES AND DOUBLE-STRANDED RNAS

(71) Applicant: ALNYLAM PHARMACEUTICALS, INC., Cambridge, MA (US)

(72) Inventors: Muthiah MANOHARAN, Cambrigde, MA (US); Rajat S. DAS, Cambrigde, MA (US); Kallanthottahill RAJEEV, Cambrigde, MA (US); Dhrubajyoti DATTA, Cambrigde, MA (US); Christopher THEILE, Cambrigde, MA (US)

(73) Assignee: ALNYLAM PHARMACEUTICALS, INC., CAMBRIDGE, MA (US)

(21) Appl. No.: 19/115,562

(22) PCT Filed: Sep. 29, 2023
(86) PCT No.: PCT/US2023/075580

§ 371 Date: Mar. 26, 2025

Related U.S. Application Data
(60) Provisional application No. 63/412,000, filed on Sep. 30, 2022.
(60) Provisional application No. 63/451,486, filed on Mar. 10, 2023.

(51) Int. Cl. C12N 15/113; A61K 9/00; A61K 31/7105; A61K 31/7115; A61K 31/712; A61K 31/7125; A61K 31/713

(52) U.S. Cl. CPC C12N 15/113; A61K 9/0019; A61K 31/7105; A61K 31/7115; A61K 31/712; A61K 31/7125; A61K 31/713; C12N 2310/14; C12N 2310/315; C12N 2310/321; C12N 2310/322; C12N 2310/33

(57) ABSTRACT

The technology described herein relates modified oligonucleotides and double-stranded RNAs, e.g., siRNAs, compositions and kits comprising them and methods of their use for inhibiting target genes.

### CROSS REFERENCE TO RELATED APPLICATIONS

This application claims benefit under 35 U.S.C. § 119 (e) of U.S. Provisional Application No. 63/412,000 filed Sep. 30, 2022 and U.S. Provisional Application No. 63/451,486 filed Mar. 10, 2023, the contents of each of which are incorporated herein by reference in their entireties.

### SEQUENCE LISTING

The instant application contains a Sequence Listing which has been submitted electronically in XML format, and is hereby incorporated by reference in its entirety. Said XML copy, created on Sep. 29, 2023 is named “051058-000100WOPT_SL.xml” and is 4,106,746 bytes in size.

### TECHNICAL FIELD

The technology described herein relates modified oligonucleotides and double-stranded RNAs, e.g., siRNAs, compositions and kits comprising them and methods of their use for inhibiting target genes.

### BACKGROUND

There remains a need in the art for oligonucleotides and siRNAs having improved activity and/or pharmacodynamics. The present disclosure addresses some of these needs.

### SUMMARY

In one aspect provided herein is a double-stranded nucleic acid (e.g., dsRNA) comprising an antisense strand and a sense strand, wherein the antisense strand and the sense strand are complementary to each other and form a double-stranded region, e.g., a double-stranded region of at least 15 base-pairs. The antisense strand comprises a ligand at its 3′-end and at least one nuclease resistant modification at each end. In other words, the antisense strand comprises a ligand at its 3′-end, at least one nuclease resistant modification at its 3′-end and at least one nuclease resistant modification at its 5′-end.

In some embodiments of any one of the aspects described herein, the sense strand also comprises at least one nuclease resistant modification. For example, the sense strand comprises at least one nuclease resistant modification at its 5′-end. In another non-limiting example, the sense strand comprises at least one nuclease resistant modification at its 3-end. In yet another non-limiting example, the sense strand comprises at least one nuclease resistant modification at its 3′-end and at least one nuclease resistant modification at its 5′-end.

As used herein, a nuclease resistant modification is a modification which makes a nucleic acid (e.g., dsRNA) more stable to degradation with nucleases (e.g., endo- or exo-nucleases). In other words, a nuclease resistant modification is a modification that inhibits or reduces cleavage of a nucleic acid by an endo- or exo-nuclease relative to the cleavage of dsRNA lacking that modification. Generally, a nuclease resistant modification is a modified internucleoside linkage, a modified sugar moiety and/or a modified nucleobase. In some embodiments of any one of the aspects described herein, the nuclease resistant modification is a modified internucleoside linkage, e.g., an internucleoside linkage other than a phosphate ester. For example, the nuclease resistant modification is a phosphorothioate or phosphorodithioate internucleoside linkage.

In some embodiments, the nuclease resistant modification is a 2′-5′-linked nucleotide, e.g.,

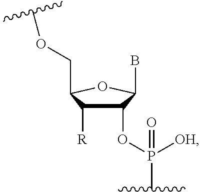

where B is an optionally modified nucleobase and R is —OH or a sugar modification described herein (e.g., —F, —OMe).

In some embodiments, the nuclease resistant modification is a L-nucleotide,

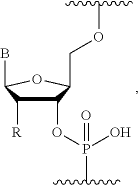

where B is an optionally modified nucleobase and R is —OH or a sugar modification described herein (e.g., —F, —OMe).

In another aspect, provided herein is a compound of Formula (I):

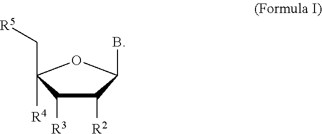

In compounds of Formula (I), B is an optionally modified nucleobase.

In compounds of Formula (I), X^{S} is O, CH₂, S, or NH. In some embodiments of any one of the aspects described herein, X^{S} is O or CH₂. For example, X^{S} is O.

In compounds of Formula (I), R⁵ is -L¹-R^{H} or —O—N(R¹³)R¹⁴, where L¹ is a bond, -L³-, C₁₋₃₀alkylene, C₂₋₃₀alkenylene, C₂₋₃₀alkynylene, *-L³-C₁₋₃₀alkylene *-L³-C₂₋₃₀alkenylene, or *-L³-C₂₋₃₀alkynylene; L³ is —O—, —N(R^{L3})—, —S—, —C(O)—, —S(O)—, —S(O)₂—, —P(X^{L3})(Y^{L3}R^{L3B})—; R^{L3} is hydrogen, optionally substituted C₁₋₃₀alkyl, optionally substituted C₁-C₃₀alkoxy, C₁₋₄haloalkyl, optionally substituted C₂₋₄alkenyl, optionally substituted C₂₋₄alkynyl, optionally substituted C₁₋₃₀alkyl-CO₂H, or a nitrogen-protecting group; X^{L2} is O or S; Y^{L3} is O, S, NH, or a bond; R^{L3B} is H or optionally substituted alkyl; * is bond to R^{H}; and R^{H} is 4-8 membered heterocyclyl comprising 1, 2 or 3 heteroatoms selected independently from N, O and S, and the heterocyclyl is optionally substituted with 1, 2, 3 or 4 independently selected substituents, and, optionally, the heterocyclyl comprises at least one nitrogen atom; or R^{H} is

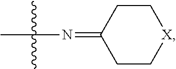

where X is O, NR^{L}, S, or CH₂; R^{L} is hydrogen, a ligand, a linker covalently bonded to one or more ligands, aliphatic and aromatic alkyl, alkylester, alkylamine, dimethylamino alkyl, alkylether, alkylthioether, heteroaromatic alkyl, allyl, vinyl, alkyl groups functionalized with disulfide, oxime, ketone, acetal, hemiacetal, cleavable peptides, or cleavable sugars; and R¹³ and R¹⁴ are independently -L²-R^{H2}, where L² is a linker; and R^{H2} is 4-8 membered heterocyclyl comprising 1, 2 or 3 heteroatoms selected independently from N, O and S, and the heterocyclyl is optionally substituted with 1, 2, 3 or 4 independently selected substituents, and, optionally at least one of R¹³ and R¹⁴ is -L²-R^{H2}.

In some compounds of Formula (I), R⁵ is -L¹-R^{H}.

In some compounds of Formula (I), L¹ is L³. For example, L¹ is —O—, —N(R^{L3})—, —S—, —C(O)—, —S(O)—, —S(O)₂—, or —P(X^{L3})(Y^{L3}R^{L3B})—.

In some compounds of Formula (I), L¹ is O or a C₁₋₃₀ alkylene. For example, L¹ is O. In some other non-limiting example, L¹ is —(CH₂)ₙ—, where n is 0 or an integer selected from 1 to 30 (e.g., n is 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30, such as n is 1, 2, 3, 4, 5 or 6). In some embodiments of any one of the aspects described herein, L¹ is methylene, i.e., —CH₂—.

In some compounds of Formula (I), R^{H} is an optionally substituted 6-membered heterocyclyl comprising a nitrogen atom and 0, 1 or 2 additional heteroatoms selected independently from N, O and S. In some compounds of Formula (I), R^{H} is

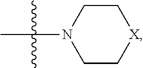

where X is O, NR^{L}, S, or CH₂; and R^{L} is hydrogen, a ligand, a linker covalently bonded to one or more ligands, aliphatic and aromatic alkyl, alkylester, alkylamine, dimethylamino alkyl, alkylether, alkylthioether, heteroaromatic alkyl, allyl, vinyl, alkyl groups functionalized with disulfide, oxime, ketone, acetal, hemiacetal, cleavable peptides, or cleavable sugars.

In some compounds of Formula (I), R^{H} is

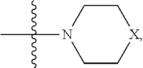

where X is O.

In some other compounds of Formula (I), R^{H} is

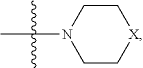

where X is NR^{L}. In some further embodiments of these compounds, R^{L} is H or aliphatic and aromatic alkyl, alkylester, alkylamine, dimethylamino alkyl, alkylether, alkylthioether, heteroaromatic alkyl, allyl, vinyl, alkyl groups functionalized with disulfide, oxime, ketone, acetal, hemiacetal, cleavable peptides, or cleavable sugars. In yet some other embodiments of these compounds, R^{L} is a ligand or linker covalently bonded to one or more independently selected ligands.

In some compounds of Formula (I), R^{H} is

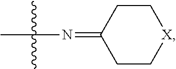

where X is O.

In some other compounds of Formula (I), R^{H} is

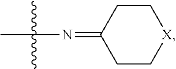

where X is NR^{L}. In some further embodiments of these compounds, R^{L} is H or aliphatic and aromatic alkyl, alkylester, alkylamine, dimethylamino alkyl, alkylether, alkylthioether, heteroaromatic alkyl, allyl, vinyl, alkyl groups functionalized with disulfide, oxime, ketone, acetal, hemiacetal, cleavable peptides, or cleavable sugars. In yet some other embodiments of these compounds, R^{L} is a ligand or linker covalently bonded to one or more independently selected ligands.

In some compounds of Formula (I), R⁵ is —O—N(R¹³)R¹⁴. It is noted, when R⁵ is —O—N(R¹³)R¹⁴, R¹³ and R¹⁴ can be same or different. Accordingly, in some compounds of Formula (I), R¹³ and R¹⁴ are same. In some other compounds of Formula (I), R¹³ and R¹⁴ are different.

In some compounds of Formula (I) described herein, one or both of R¹³ and R¹⁴ can be -L²-R^{H2}.

In some compounds of Formula (I), L² is a bond or an optionally substituted alkylene. For example, L² is a bond. In some other compounds of Formula (I), L² is —Z—(CH₂)ₘ—, where Z is absent, aryl, heteroaryl, cycloalkyl or heterocyclyl; and m is 0 or an integer selected from 1 to 20 (e.g., m is 1, 2, 3, 4, 5, 6, 7, 8, 9, 10, 11, 12, 13, 14 or 15, such as m is 1, 2, 3, 4, 5 or 6). For example, L² is —(CH₂)ₘ— or —(CH₂)ₘ-phenyl-.

In some compounds of Formula (I), at least one (e.g., one or both) of R¹³ and R¹⁴ is —

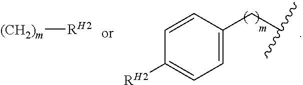

In some compounds of Formula (I), R^{H2} is an optionally substituted 6-membered heterocyclyl comprising a nitrogen atom and 0, 1 or 2 additional heteroatoms selected independently from N, O and S. For example, R^{H2} is

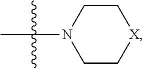

where X is O, NR^{L}, S, or CH₂; and R^{L} is hydrogen, a ligand, a linker covalently bonded to one or more ligands, aliphatic and aromatic alkyl, alkylester, alkylamine, dimethylamino alkyl, alkylether, alkylthioether, heteroaromatic alkyl, allyl, vinyl, alkyl groups functionalized with disulfide, oxime, ketone, acetal, hemiacetal, cleavable peptides, or cleavable sugars.

In some compounds of Formula (I), R^{H2} is

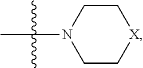

where X is O.

In some other compounds of Formula (I), R^{H2} is

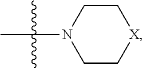

where X is NR^{L}. In some further embodiments, R^{L} is H or aliphatic and aromatic alkyl, alkylester, alkylamine, dimethylamino alkyl, alkylether, alkylthioether, heteroaromatic alkyl, allyl, vinyl, alkyl groups functionalized with disulfide, oxime, ketone, acetal, hemiacetal, cleavable peptides, or cleavable sugars. In yet some other embodiments of any one of the aspects described herein, R^{L} is a ligand or linker covalently bonded to one or more independently selected ligands.

In some compounds of Formula (I), one of R¹³ and R¹⁴ is an optionally substituted C₁-C₆alkyl. For example, one of R¹³ and R¹⁴ is methyl.

In some embodiments of compounds of Formula (I), one of R¹³ and R¹⁴ is -L²-R^{H2} and the other is an optionally substituted C₁-C₆alkyl (e.g., methyl).

In some compounds of Formula (I), one of R¹³ and R¹⁴ is

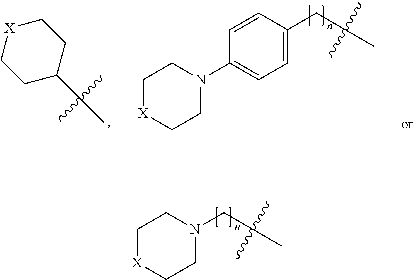

and the other of R¹³ and R¹⁴ is C₁-C₆alkyl,

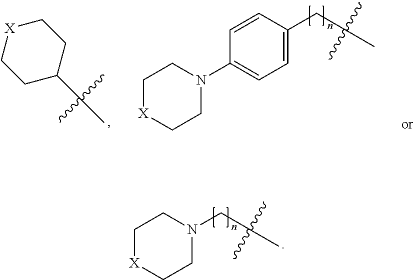

In some embodiments of any one of the aspects described herein, X^{P} is —P(X)(OR^{V})₂, where each X is independently O or S, and each R^{V} is H or oxygen protecting group. For example, R⁵ is —CH—CH—P(X)(OR^{V})₂, where each X is independently O or S, and each RY is independently H or an oxygen protecting group.

In some cases, X is O. For example, R⁵ is —CH═CH—P(O)(OR^{V})₂. In some embodiments, R⁵ is —CH═CH—P(O)(OH)₂. In some other embodiments, R⁵ is —CH═CH—P(O)(OR^{V})₂, where each R^{V} is independently an oxygen protecting group. For example, R⁵ is —CH═CH—P(O)(OR^{V})₂, where each R^{V} is independently 4-pentenyloxymethyl (POM). In yet some other embodiments, R⁵ is —CH═CH—P(O)(OH)(OR^{V}), where R^{V} is an oxygen protecting group.

In some cases, X is S. For example, R⁵ is —CH═CH—P(S)(OR^{V})₂. In some embodiments, R⁵ is —CH═CH—P(S)(OH)₂. In some other embodiments, R⁵ is —CH═CH—P(S)(OR^{V})₂, where each R^{V} is independently an oxygen protecting group. For example, R⁵ is —CH═CH—P(S)(OR^{V})₂, where each R^{V} is independently 4-pentenyloxymethyl (POM). In yet some other embodiments, R⁵ is —CH═CH—P(S)(OH)(OR^{V}), where R^{V} is an oxygen protecting group.

In compounds of Formula (I), R² is hydrogen, hydroxyl, protected hydroxyl, phosphate group, reactive phosphorous group, halogen, optionally substituted C₁₋₃₀ alkyl, optionally substituted C₂₋₃₀alkenyl, optionally substituted C₂₋₃₀alkynyl, optionally substituted C₁₋₃₀ alkoxy (e.g., methoxy or 2′-methoxyethoxy), alkoxyalkyl (e.g., 2-methoxyethyl), alkoxyalkylamine, alkoxyoxycarboxylate, amino, alkylamino, dialkylamino, 5-8 membered heterocyclyl, —O—C₄₋₃₀alkyl-ON(CH₂R⁸)(CH₂R⁹), or —O—C₄₋₃₀alkyl-ON(CH₂R⁸)(CH₂R⁹), a ligand, a linker covalently bonded to one or more ligands, a solid support, a linker or a linker covalently bonded a solid support. In some compounds of Formula (I), R² can be hydrogen, hydroxyl, protected hydroxyl, phosphate group, reactive phosphorous group, halogen, optionally substituted C₁₋₃₀ alkyl, optionally substituted C₂₋₃₀alkenyl, optionally substituted C₂₋₃₀alkynyl, optionally substituted C₁₋₃₀ alkoxy (e.g., methoxy or 2′-methoxyethoxy), alkoxyalkyl (e.g., 2-methoxyethyl), amino, alkylamino, dialkylamino, —O—C₄₋₃₀alkyl-ON(CH₂R⁸)(CH₂R⁹), or —O—C₄₋₃₀alkyl-ON(CH₂R⁸)(CH₂R⁹), alkoxyoxycarboxylate. In some compounds of Formula (I), R² can be hydrogen, hydroxyl, halogen, protected hydroxyl, phosphate group, reactive phosphorous group, optionally substituted C₁₋₃₀ alkyl, optionally substituted C₂₋₃₀ alkenyl, optionally substituted C₂₋₃₀ alkynyl, optionally substituted C₁₋₃₀alkoxy (e.g., methoxy or 2′-methoxyethoxy), alkoxyalkyl (e.g., methoxyethyl), amino, alkylamino, dialkylamino, —O—C₄₋₃₀alkyl-ON(CH₂R⁸)(CH₂R⁹), —O—C₄₋₃₀alkyl-ON(CH₂R⁸)(CH₂R⁹). In some compounds of Formula (I), R² is hydrogen, hydroxyl, protected hydroxyl, fluoro, methoxy, ethoxy, 2-methoxyethoxy, C₆₋₂₄ alkyl (e.g., n-C₆₋₂₄ alkyl), or a reactive phosphorous group. In some compounds of Formula (I), R² is hydrogen, hydroxyl, protected hydroxyl, fluoro, methoxy, ethoxy, 2-methoxyethoxy, or a reactive phosphorous group. In some compounds of Formula (I), R² is hydrogen, hydroxyl, protected hydroxyl, fluoro or methoxy. In some compounds of Formula (I), R² is hydrogen, fluoro or methoxy.

In compounds of Formula (I), R³ is hydrogen, hydroxyl, protected hydroxyl, phosphate group, reactive phosphorous group, halogen, optionally substituted C₁₋₃₀ alkyl, optionally substituted C₂₋₃₀alkenyl, optionally substituted C₂₋₃₀alkynyl, optionally substituted C₁₋₃₀ alkoxy (e.g., methoxy or 2′-methoxyethoxy), alkoxyalkyl (e.g., 2-methoxyethyl), alkoxyalkylamine, alkoxyoxycarboxylate, amino, alkylamino, dialkylamino, 5-8 membered heterocyclyl, —O—C₄₋₃₀alkyl-ON(CH₂R⁸)(CH₂R⁹), or —O—C₄₋₃₀alkyl-ON(CH₂R⁸)(CH₂R⁹), a ligand, a linker covalently bonded to one or more ligands, a solid support, a linker or a linker covalently bonded a solid support. In some compounds of Formula (I), R³ can be hydrogen, hydroxyl, protected hydroxyl, phosphate group, reactive phosphorous group, halogen, optionally substituted C₁₋₃₀ alkyl, optionally substituted C₂₋₃₀alkenyl, optionally substituted C₂₋₃₀alkynyl, optionally substituted C₁₋₃₀ alkoxy (e.g., methoxy or 2′-methoxyethoxy), alkoxyalkyl (e.g., 2-methoxyethyl), amino, alkylamino, dialkylamino, —O—C₄₋₃₀alkyl-ON(CH₂R⁸)(CH₂R⁹), or —O—C₄₋₃₀alkyl-ON(CH₂R⁸)(CH₂R⁹), alkoxyoxycarboxylate. For example, R³ can be reactive phosphorous group, hydrogen, hydroxyl, halogen, protected hydroxyl, phosphate group, optionally substituted C₁₋₃₀ alkyl, optionally substituted C₂₋₃₀ alkenyl, optionally substituted C₂₋₃₀ alkynyl, optionally substituted C₁₋₃₀alkoxy (e.g., methoxy or 2′-methoxyethoxy), alkoxyalkyl (e.g., methoxyethyl), amino, alkylamino, dialkylamino, —O—C₄₋₃₀alkyl-ON(CH₂R⁸)(CH₂R⁹), —O—C₄₋₃₀alkyl-ON(CH₂R⁸)(CH₂R⁹). In some compounds of Formula (I), R³ is a reactive phosphorous group, hydrogen, hydroxyl, protected hydroxyl, fluoro, methoxy, ethoxy, 2-methoxyethoxy, or C₆₋₂₄ alkyl (e.g., n-C₆₋₂₄ alkyl). In some compounds of Formula (I), R³ is a reactive phosphorous group, hydrogen, hydroxyl, protected hydroxyl, fluoro, methoxy, ethoxy, or 2-methoxyethoxy. In some compounds of Formula (I), R³ is a reactive phosphorous group. In some compounds of Formula (I), R³ is a phosphoramidite group such as 3′-[(2-cyanoethyl)-(N,N-diisopropyl)]-phosphoramidite, 3′-[(2-cyanoethyl)-(N,N-diisopropyl)]-phosphoramidite, or 3′-[(ß-thiobenzoylethyl)-(1-pyrrolidinyl)]-thiophosphoramidite).

It is noted that in compounds of Formula (I) no more than one of R² and R³ is a reactive phosphorous group. For example, only R³ is a reactive phosphorous group.

In some compounds of Formula (I), R⁴ is hydrogen, optionally substituted C₁₋₆alkyl, optionally substituted C₂₋₆alkenyl, optionally substituted C₂₋₆alkynyl, or optionally substituted C₁₋₆alkoxy. For example, R⁴ in Formula (I) is H.

In some compounds of Formula (I), R⁴ and R² taken together are 4′-C(R¹⁰R¹¹)ᵥ—Y-2′ or 4′-Y—C(R¹⁰R¹¹)ᵥ-2′;Y is —O—, —CH₂—, —CH(Me)—, —C(CH₃)₂—, —S—, —N(R¹²)—, —C(O)—, —C(S)—, —S(O)—, —S(O)₂—, —OC(O)—, —C(O)O—, —N(R¹²)C(O)—, or —C(O)N(R¹²)—; R¹⁰ and R¹¹ independently are H, optionally substituted C₁-C₆alkyl, optionally substituted C₂-C₆alkenyl or optionally substituted C₂-C₆alkynyl; R¹² is hydrogen, optionally substituted C₁₋₃₀alkyl, optionally substituted C₁-C₃₀alkoxy, C₁₋₄haloalkyl, optionally substituted C₂₋₄alkenyl, optionally substituted C₂₋₄alkynyl, optionally substituted C₁₋₃₀alkyl-CO₂H, or a nitrogen-protecting group; and v is 1, 2 or 3. For example, R² and R⁴ taken together are 4′-C(R¹⁰R¹¹)ᵥ—Y-2′ or 4′-Y—C(R¹⁰R¹¹)ᵥ-2′.

In some compounds of Formula (I), R⁴ and R³ taken together with the atoms to which they are attached form an optionally substituted C₃₋₈cycloalkyl, optionally substituted C₃₋₈cycloalkenyl, or optionally substituted 3-8 membered heterocyclyl.

In some embodiments of any one of the aspects described herein, the compound of Formula (I) is a compound selected from formulae (I-A)-(I-D):

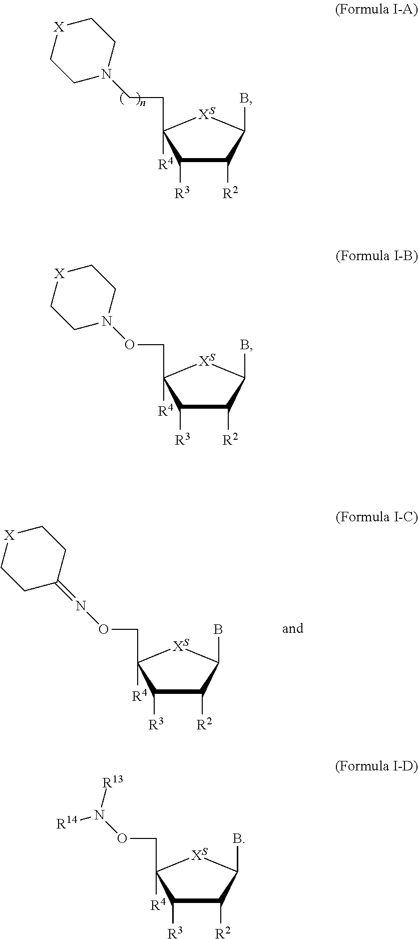

In some compounds of Formula (I), (I-A), (I-B), (I-C) or (I-D), X^{S} is O; R² and R⁴ taken together are 4′-Y—C(R¹⁰R¹¹)ᵥ-2′; and R³ is a reactive phosphorous group, hydroxyl or protected hydroxyl.

In some compounds of Formula (I), (I-A), (I-B), (I-C) or (I-D), X^{S} is O; R² is H, —OMe, —F; R³ is a reactive phosphorous group, hydroxyl or protected hydroxyl; and R⁴ is H.

In some embodiments of the various aspects described herein, the compound of Formula (I) is of Formula (I-E):

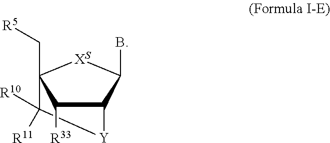

In some compounds of Formula (I-E), R³ is a reactive phosphorous group, hydroxyl, or protected hydroxyl; R⁵ is -L¹-R^{H}; and X^{S}, B, Y, R¹⁰ and R¹¹ are as defined for Formula (I).

In some embodiments of the various aspects described herein, the compound of Formula (I-E) is of Formula (I-E¹) or (I-E²):

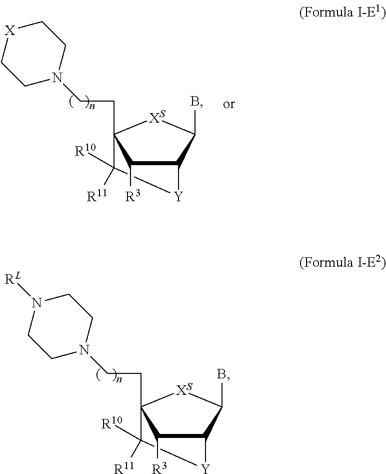

where n is 0 or an integer selected from 1 to 30 (e.g., n is 1, 2, 3, 4, 5, 6, 7, 8, 9, 10, 11, 12, 13, 14, 15, 16, 16, 17, 18, 19, 20, 21, 22, 23, 24, 25, 26, 27, 28, 29 or 30, such as n is 1, 2, 3, 4, 5 or 6, preferably n is 0 or 1); and
R^{L} is H, a ligand, a linker covalently bonded to one or more ligands, aliphatic and aromatic alkyl, alkylester, alkylamine, dimethylamino alkyl, alkylether, alkylthioether, heteroaromatic alkyl, allyl, vinyl, alkyl groups functionalized with disulfide, oxime, ketone, acetal, hemiacetal, cleavable peptides, or cleavable sugars.

In some compounds of Formula (I-E¹) or (I-E²), X^{s} is O; Y is O; and one of R¹⁰ and R¹¹ is H and the other is H, a ligand, a linker covalently bonded to one or more ligands, aliphatic and aromatic alkyl, alkylester, alkylamine, dimethylamino alkyl, alkylether, alkylthioether, heteroaromatic alkyl, allyl, vinyl, alkyl groups functionalized with disulfide, oxime, ketone, acetal, hemiacetal, cleavable peptides, or cleavable sugars. For example, one of R¹⁰ and R¹¹ is H and the other is H or linear, cyclic or branched alkyl (e.g., methyl, propyl, isopropyl, etc.)

In some embodiments of the various aspects described herein, the compound of Formula (I-E) is of Formula (I-Ea), (I-Eb) or (I-Ec):

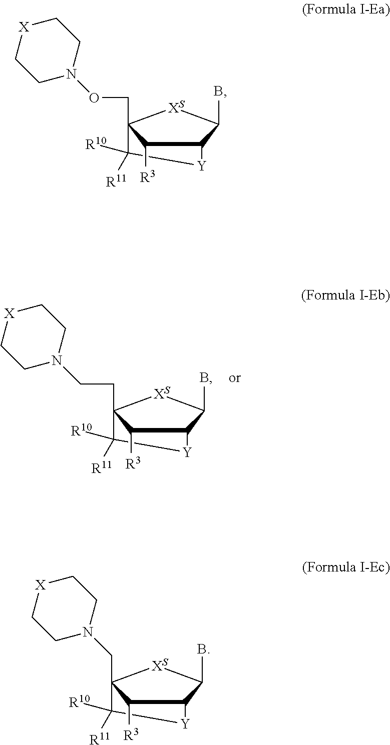

In some compounds of Formula (I-E), R³ is a reactive phosphorous group, hydroxyl, or protected hydroxyl; and X^{S}, B, Y, R¹⁰ and R¹¹ are as defined for Formula (I).

In some embodiments of the various aspects described herein, the compound of Formula (I-E) is of Formula (I-E³) or (I-E⁴):

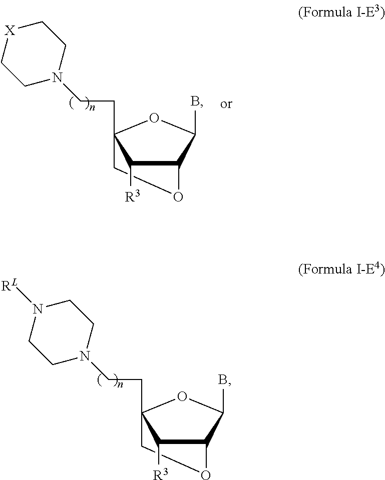

where n is 0 or an integer selected from 1 to 30 (e.g., n is 1, 2, 3, 4, 5, 6, 7, 8, 9, 10, 11, 12, 13, 14, 15, 16, 16, 17, 18, 19, 20, 21, 22, 23, 24, 25, 26, 27, 28, 29 or 30, such as n is 1, 2, 3, 4, 5 or 6, preferably n is 0 or 1); and
R^{L} is H, a ligand, a linker covalently bonded to one or more ligands, aliphatic and aromatic alkyl, alkylester, alkylamine, dimethylamino alkyl, alkylether, alkylthioether, heteroaromatic alkyl, allyl, vinyl, alkyl groups functionalized with disulfide, oxime, ketone, acetal, hemiacetal, cleavable peptides, or cleavable sugars.

In some embodiments of the various aspects described herein, the compound of Formula (I-Ea) is of Formula (I-Ed) or (I-Ee):

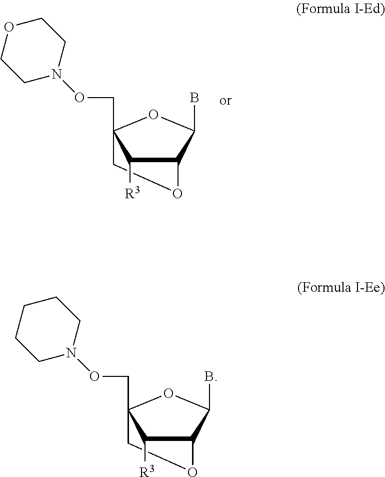

In some embodiments of the various aspects described herein, the compound of Formula (I-Eb) is of Formula (I-Ef):

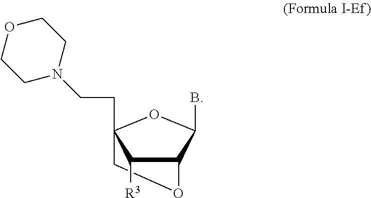

In some embodiments of the various aspects described herein, the compound of Formula (I-Ec) is of Formula (I-Eg):

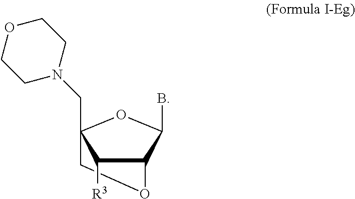

In some compounds of Formula (I-Ed), (I-E³), (I-E⁴), (I-Ee), (I-Ef) and/or (I-Eg), R³ is a reactive phosphorous group, hydroxyl or protected hydroxyl. For example, in some compounds of Formula (I-Ed), (I-E³), (I-E⁴), (I-Ee), (I-Ef) and/or (I-Eg), R³ is —OP(OR^{P})(N(R^{P2})₂), where R^{P} is cyanoethyl (—CH₂CH₂CN) and each R^{P2} is isopropyl.

In some embodiments of the various aspects described herein, R⁵ is not morpholin-4-yl unless R⁴ and R² taken together are 4′-C(R¹⁰R¹¹)ᵥ—Y-2′ or 4′-Y—C(R¹⁰R¹¹)ᵥ-2′.

In some embodiments of the various aspects described herein, when R² is H, hydroxyl, protected hydroxyl, alkoxy, or halogen; R³ is hydroxyl, protected hydroxyl, or reactive phosphorous group; R⁴ is H; and X^{S} is O, then R⁵ is not morpholin-4-yl.

### Oligonucleotides

The compounds of Formula (I) are useful in the synthesis oligonucleotides. Accordingly, in another aspect, provided herein is an oligonucleotide prepared using a compound of Formula (I). For example, an oligonucleotide comprising a nucleoside of Formula (II). Accordingly, in another aspect, provided herein is an oligonucleotide comprising at least one nucleoside of Formula (II):

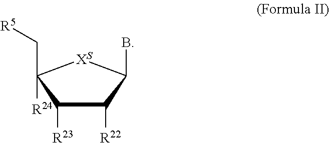

In nucleosides of Formula (II), B is an optionally modified nucleobase.

In nucleosides of Formula (II), X^{S} is O, CH₂, S, or NH. In some embodiments of any one of the aspects described herein, X^{S} is O or CH₂. For example, X^{S} is O.

In nucleosides of Formula (II), R⁵ is -L¹-R^{H} or —O—N(R¹³)R¹⁴, where L¹ is a bond, -L³-, C₁₋₃₀alkylene, C₂₋₃₀alkenylene, C₂₋₃₀alkynylene, *-L³-C₁₋₃₀alkylene *-L³-C₂₋₃₀alkenylene, or *-L³-C₂₋₃₀alkynylene; L³ is —O—, —N(R^{L3})—, —S—, —C(O)—, —S(O)—, —S(O)₂—, —P(X^{L3})(Y^{L3}R^{L3B})—; R^{L3} is hydrogen, optionally substituted C₁₋₃₀alkyl, optionally substituted C₁-C₃₀alkoxy, C₁₋₄haloalkyl, optionally substituted C₂₋₄alkenyl, optionally substituted C₂₋₄alkynyl, optionally substituted C₁₋₃₀alkyl-CO₂H, or a nitrogen-protecting group; X^{L2} is O or S; Y^{L3} is O, S, NH, or a bond; R^{L3B} is H or optionally substituted alkyl; * is bond to R^{H}; and R^{H} is 4-8 membered heterocyclyl comprising 1, 2 or 3 heteroatoms selected independently from N, O and S, and the heterocyclyl is optionally substituted with 1, 2, 3 or 4 independently selected substituents, and, optionally, the heterocyclyl comprises at least one nitrogen atom, or R^{H} is

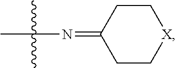

where X is O, NR^{L}, S, or CH₂; R^{L} is hydrogen, a ligand, a linker covalently bonded to one or more ligands, aliphatic and aromatic alkyl, alkylester, alkylamine, dimethylamino alkyl, alkylether, alkylthioether, heteroaromatic alkyl, allyl, vinyl, alkyl groups functionalized with disulfide, oxime, ketone, acetal, hemiacetal, cleavable peptides, or cleavable sugars; and R¹³ and R¹⁴ are independently-L²-R^{H2}, where L² is a linker; and R^{H2} is 4-8 membered heterocyclyl comprising 1, 2 or 3 heteroatoms selected independently from N, O and S, and the heterocyclyl is optionally substituted with 1, 2, 3 or 4 independently selected substituents, and, optionally at least one of R¹³ and R¹⁴ is -L²-R^{H2}.

In some nucleosides of Formula (II), R⁵ is -L¹-R^{H}.

In some nucleosides of Formula (II), L¹ is L³. For example, L¹ is —O—, —N(R^{L3})—, —S—, —C(O)—, —S(O)—, —S(O)₂—, or —P(X^{L3})(Y^{L3}R^{L3B})—.

In some nucleosides of Formula (II), L¹ is O or an optionally substituted alkylene. For example, L¹ is O. In some other non-limiting example, L¹ is —(CH₂)ₙ—, where n is 0 or an integer selected from 1 to 20 (e.g., n is 1, 2, 3, 4, 5, 6, 7, 8, 9, 10, 11, 12, 13, 14 or 15, such as n is 1, 2, 3, 4, 5 or 6). In some embodiments of any one of the aspects described herein, L¹ is methylene, i.e., —CH₂—.

In some nucleosides of Formula (II), R^{H} is an optionally substituted 6-membered heterocyclyl comprising a nitrogen atom and 0, 1 or 2 additional heteroatoms selected independently from N, O and S. For example, R^{H} is

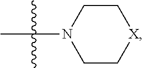

where X is O, NR^{L}, S, or CH₂; and R^{L} is hydrogen, a ligand, a linker covalently bonded to one or more ligands, aliphatic and aromatic alkyl, alkylester, alkylamine, dimethylamino alkyl, alkylether, alkylthioether, heteroaromatic alkyl, allyl, vinyl, alkyl groups functionalized with disulfide, oxime, ketone, acetal, hemiacetal, cleavable peptides, or cleavable sugars.

In some nucleosides of Formula (II), R^{H} is

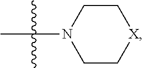

where X is O.

In some other nucleosides of Formula (II), R^{H} is

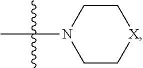

where X is NR^{L}. In some further embodiments of these compounds, R^{L} is H or aliphatic and aromatic alkyl, alkylester, alkylamine, dimethylamino alkyl, alkylether, alkylthioether, heteroaromatic alkyl, allyl, vinyl, alkyl groups functionalized with disulfide, oxime, ketone, acetal, hemiacetal, cleavable peptides, or cleavable sugars. In yet some other embodiments of these compounds, R^{L} is a ligand or linker covalently bonded to one or more independently selected ligands.

In some nucleosides of Formula (II), R^{H} is

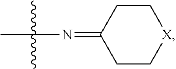

where X is O.

In some other nucleosides of Formula (II), R^{H} is

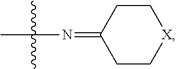

where X is NR^{L}. In some further embodiments of these compounds, R^{L} is H or aliphatic and aromatic alkyl, alkylester, alkylamine, dimethylamino alkyl, alkylether, alkylthioether, heteroaromatic alkyl, allyl, vinyl, alkyl groups functionalized with disulfide, oxime, ketone, acetal, hemiacetal, cleavable peptides, or cleavable sugars. In yet some other embodiments of these compounds, R^{L} is a ligand or linker covalently bonded to one or more independently selected ligands.

In some nucleosides of Formula (II), R⁵ is —O—N(R¹³)R¹⁴. It is noted, when R⁵ is —O—N(R¹³)R¹⁴, R¹³ and R¹⁴ can be same or different. Accordingly, in some nucleosides of Formula (II), R¹³ and R¹⁴ are same. In some other compounds of Formula (I), R¹³ and R¹⁴ are different.

In some nucleosides of Formula (II) described herein, one or both of R¹³ and R¹⁴ can be -L²-R^{H2}.

In some nucleosides of Formula (II), L² is a bond or an optionally substituted alkylene. For example, L² is a bond. In some other compounds of Formula (I), L² is —Z—(CH₂)ₘ—, where Z is absent, aryl, heteroaryl, cycloalkyl or heterocyclyl; and m is 0 or an integer selected from 1 to 20 (e.g., m is 1, 2, 3, 4, 5, 6, 7, 8, 9, 10, 11, 12, 13, 14 or 15, such as m is 1, 2, 3, 4, 5 or 6). For example, L² is —(CH₂)ₘ— or —(CH₂)ₘ-phenyl-.

In some nucleosides of Formula (II), at least one (e.g., one or both) of R¹³ and R¹⁴ is

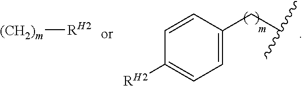

In some compounds of Formula (I), R^{H2} is an optionally substituted 6-membered heterocyclyl comprising a nitrogen atom and 0, 1 or 2 additional heteroatoms selected independently from N, O and S. For example, R^{H2} is

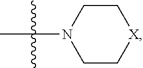

where X is O, NR^{L}, S, or CH₂; and R^{L} is hydrogen, a ligand, a linker covalently bonded to one or more ligands, aliphatic and aromatic alkyl, alkylester, alkylamine, dimethylamino alkyl, alkylether, alkylthioether, heteroaromatic alkyl, allyl, vinyl, alkyl groups functionalized with disulfide, oxime, ketone, acetal, hemiacetal, cleavable peptides, or cleavable sugars.

In some nucleosides of Formula (II), R^{H2} is

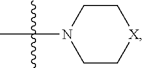

where X is O.

In some other nucleosides of Formula (II), R^{H2} is

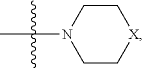

where X is NR^{L}. In some further embodiments, R^{L} is H or aliphatic and aromatic alkyl, alkylester, alkylamine, dimethylamino alkyl, alkylether, alkylthioether, heteroaromatic alkyl, allyl, vinyl, alkyl groups functionalized with disulfide, oxime, ketone, acetal, hemiacetal, cleavable peptides, or cleavable sugars. In yet some other embodiments of any one of the aspects described herein, R^{L} is a ligand or linker covalently bonded to one or more independently selected ligands.

In some nucleosides of Formula (II), one of R¹³ and R¹⁴ is an optionally substituted C₁-C₆alkyl. For example, one of R¹³ and R¹⁴ is methyl.

In some embodiments of nucleosides of Formula (II), one of R¹³ and R¹⁴ is -L²-R^{H2} and the other is an optionally substituted C₁-C₆alkyl (e.g., methyl).

In some nucleosides of Formula (II), one of R¹³ and R¹⁴ is

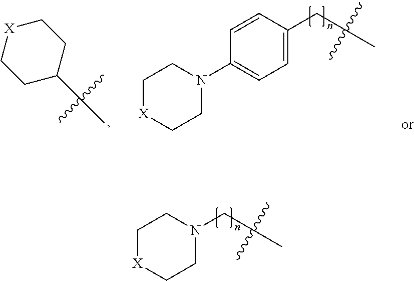

and the other of R¹³ and R¹⁴ is C₁-C₆alkyl,

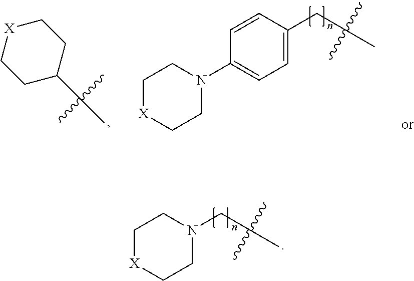

In some nucleosides of Formula (II), X^{P} is —P(X)(OR^{V})₂, where each X is independently O or S, and each R^{V} is H or oxygen protecting group. For example, in some nucleosides of Formula (II), R⁵ is —CH═CH—P(X)(OR^{V})₂, where each X is independently O or S, and each R^{V} is independently H or an oxygen protecting group.

In some nucleosides of Formula (II), X is O. For example, in some nucleosides of Formula (II), R⁵ is —CH═CH—P(O)(OR^{V})₂. In some nucleosides of Formula (II), R⁵ is —CH═CH—P(O)(OH)₂. In some other in nucleosides of Formula (II), R⁵ is —CH═CH—P(O)(OR^{V})₂, where each R^{V} is independently an oxygen protecting group. For example, in some nucleosides of Formula (II), R⁵ is —CH═CH—P(O)(OR^{V})₂, where each R^{V} is independently 4-pentenyloxymethyl (POM). In yet some nucleosides of Formula (II), R⁵ is —CH═CH—P(O)(OH)(OR^{V}), where R^{V} is an oxygen protecting group.

In some nucleosides of Formula (II), X is S. For example, in some nucleosides of Formula (II), R⁵ is —CH═CH—P(S)(OR^{V})₂. In some nucleosides of Formula (II), R⁵ is —CH═CH—P(S)(OH)₂. In some other embodiments, R⁵ is —CH═CH—P(S)(OR^{V})₂, where each R^{V} is independently an oxygen protecting group. For example, in some nucleosides of Formula (II), R⁵ is —CH═CH—P(S)(OR^{V})₂, where each RY is independently 4-pentenyloxymethyl (POM). In yet some other nucleosides of Formula (II), R⁵ is —CH═CH—P(S)(OH)(OR^{V}), where R^{V} is an oxygen protecting group.

In nucleosides of Formula (II), R²² can be a hydroxyl, protected hydroxyl, halogen, optionally substituted C₁₋₃₀ alkoxy (e.g., methoxy, 2-methoxyethoxy), alkoxyalkyl (e.g., 2-methoxyethyl), hydrogen, optionally substituted C₁₋₃₀ alkyl, optionally substituted C₂₋₃₀alkenyl, optionally substituted C₂₋₃₀alkynyl, alkoxyalkylamine, alkoxyoxycarboxylate, amino, alkylamino, dialkylamino, 5-8 membered heterocyclyl, —O—C₄₋₃₀alkyl-ON(CH₂R⁸)(CH₂R⁹), —O—C₄₋₃₀alkyl-ON(CH₂R⁸)(CH₂R⁹), a ligand, a linker covalently bonded to one or more ligands or a bond to an internucleotide linkage to a subsequent nucleoside, provided that at least one, and only one, of R²² and R²³ is a bond to an internucleotide linkage to a subsequent nucleotide. In some embodiments, R²² is a hydroxyl, protected hydroxyl, halogen, optionally substituted C₁₋₃₀ alkoxy (e.g., methoxy, 2-methoxyethoxy), alkoxyalkyl (e.g., 2-methoxyethyl), hydrogen, alkoxyalkylamine, alkoxyoxycarboxylate, amino, alkylamino, or dialkylamino. For example, R²² is a hydroxyl, protected hydroxyl, halogen, or optionally substituted C₁₋₃₀ alkoxy (e.g., methoxy, 2-methoxyethoxy). In some embodiments, R²² is hydrogen, fluoro or methoxy.

In nucleosides of Formula (II), R²³ can be a bond to an internucleotide linkage to a subsequent nucleoside, hydroxyl, protected hydroxyl, halogen, optionally substituted C₂₋₃₀alkynyl, optionally substituted C₁₋₃₀ alkoxy (e.g., methoxy, 2-methoxyethoxy), alkoxyalkyl (e.g., 2-methoxyethyl), hydrogen, optionally substituted C₁₋₃₀ alkyl, optionally substituted C₂₋₃₀alkenyl, alkoxyalkylamine, alkoxyoxycarboxylate, amino, alkylamino, dialkylamino, 5-8 membered heterocyclyl, —O—C₄₋₃₀alkyl-ON(CH₂R⁸)(CH₂R⁹), —O—C₄₋₃₀alkyl-ON(CH₂R⁸)(CH₂R⁹), phosphate group, a ligand, or a linker covalently bonded to one or more ligands, provided that at least one, and only one, of R²² and R²³ is a bond to an internucleotide linkage to a subsequent nucleotide. In some embodiments, R²³ is a bond to an internucleotide linkage to a subsequent nucleotide.

In nucleosides of Formula (II), R²⁴ can be hydrogen, optionally substituted C₁₋₆alkyl, optionally substituted C₂₋₆alkenyl, optionally substituted C₂₋₆alkynyl, or optionally substituted C₁-6alkoxy. For example, R²⁴ in nucleosides of Formula (II) is H.

In some nucleosides of Formula (II), R²⁴ and R₂₂ taken together are 4′-C(R¹⁰R¹¹)ᵥ—Y-2′ or 4′-Y—C(R¹⁰R¹¹)ᵥ-2′;Y is —O—, —CH₂—, —CH(Me)—, —C(CH₃)₂—, —S—, —N(R¹²)—, —C(O)—, —C(S)—, —S(O)—, —S(O)₂—, —OC(O)—, —C(O)O—, —N(R¹²)C(O)—, or —C(O)N(R¹²)—; R¹⁰ and R¹¹ independently are H, optionally substituted C₁-C₆alkyl, optionally substituted C₂-C₆alkenyl or optionally substituted C₂-C₆alkynyl; R¹² is hydrogen, optionally substituted C₁₋₃₀alkyl, optionally substituted C₁-C₃₀alkoxy, C₁₋₄haloalkyl, optionally substituted C₂₋₄alkenyl, optionally substituted C₂₋₄alkynyl, optionally substituted C₁₋₃₀alkyl-CO₂H, or a nitrogen-protecting group; and v is 1, 2 or 3. For example, R²² and R²⁴ taken together are 4′-C(R¹⁰R¹¹)ᵥ—Y-2′ or 4′-Y—C(R¹⁰R¹¹)ᵥ-2.

In some embodiments of any one of the aspects described herein, the nucleoside of Formula (II) is a nucleoside selected from formulae (II-A)-(II-D):

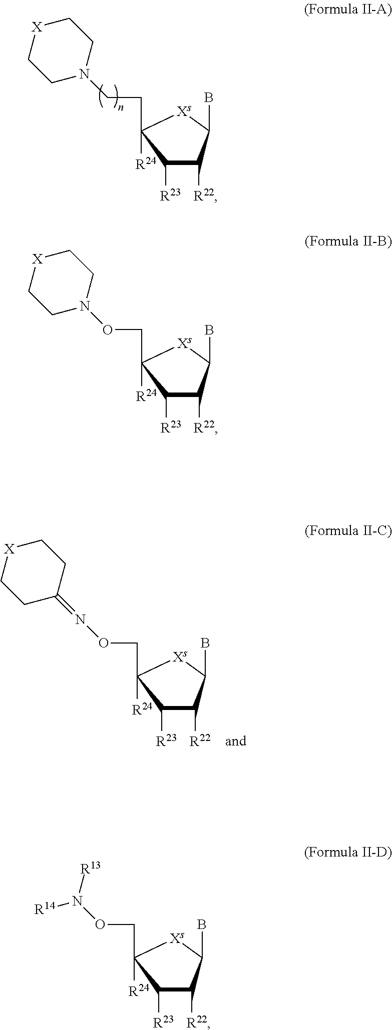

In some nucleosides of Formula (II), (II-A), (II-B), (II-C) or (II-D), X^{S} is O; R²² and R²⁴ taken together are 4′-Y—C(R¹⁰R¹¹)ᵥ-2′; and R²³ is a bond to an internucleotide linkage to a subsequent nucleoside.

In some nucleosides of Formula (II), (II-A), (II-B), (II-C) or (II-D), X^{S} is O; R²² is H, —OMe or —F; R²³ is a bond to an internucleotide linkage to a subsequent nucleoside; and R²⁴ is H.

It is noted that in nucleosides of Formula (II), (II-A), (II-B), (II-C) or (II-D) no more than one of R²² and R²³ is a bond to an internucleotide linkage to a subsequent nucleotide. For example, only R²³ is a bond to an internucleotide linkage to a subsequent nucleotide. In some other non-limiting examples, only R²² is a bond to an internucleotide linkage to a subsequent nucleotide. Preferably, only R²³ is a bond to an internucleotide linkage to a subsequent nucleotide.

In some embodiments of any one of the aspects described herein, the nucleoside of Formula (II) is of Formula (II-E)

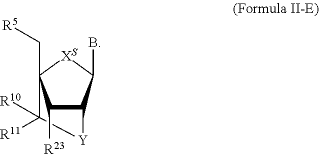

In some nucleosides of Formula (II-E), R²³ is a bond to an internucleotide linkage to a subsequent nucleoside; R⁵ is -L¹-R^{H}; and X^{S}, B, Y, R¹⁰ and R¹¹ are as defined for Formula (II).

In some embodiments of any one of the aspects described herein, the nucleoside of Formula (II-E) is of Formula (II-E¹) or (II-E²):

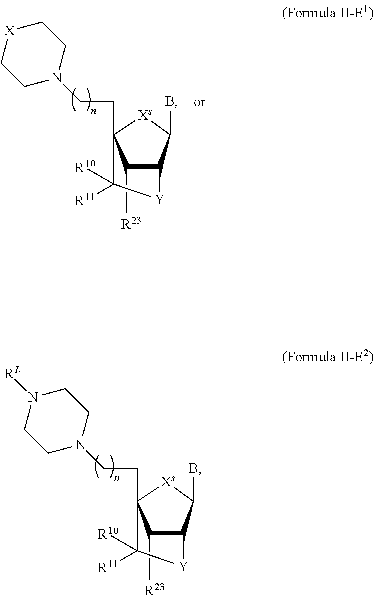

wherein
n is 0 or an integer selected from 1 to 30 (e.g., n is 1, 2, 3, 4, 5, 6, 7, 8, 9, 10, 11, 12, 13, 14, 15, 16, 16, 17, 18, 19, 20, 21, 22, 23, 24, 25, 26, 27, 28, 29 or 30, such as n is 1, 2, 3, 4, 5 or 6, preferably n is 0 or 1); and
R^{L} is H, a ligand, a linker covalently bonded to one or more ligands, aliphatic and aromatic alkyl, alkylester, alkylamine, dimethylamino alkyl, alkylether, alkylthioether, heteroaromatic alkyl, allyl, vinyl, alkyl groups functionalized with disulfide, oxime, ketone, acetal, hemiacetal, cleavable peptides, or cleavable sugars,
In some embodiments of any one of the aspects described herein, in nucleoside of Formula (II-E¹) or (II-E²), X^{s} is O; Y is O; and one of R¹⁰ and R¹¹ is H and the other is H, a ligand, a linker covalently bonded to one or more ligands, aliphatic and aromatic alkyl, alkylester, alkylamine, dimethylamino alkyl, alkylether, alkylthioether, heteroaromatic alkyl, allyl, vinyl, alkyl groups functionalized with disulfide, oxime, ketone, acetal, hemiacetal, cleavable peptides, or cleavable sugars. For example, one of R¹⁰ and R¹¹ is H and the other is H or linear, cyclic or branched alkyl (e.g., methyl, propyl, isopropyl, etc.)

In some embodiments of any one of the aspects described herein, the nucleoside of Formula (II-E) is of Formula (II-Ea), (II-Eb) or (II-Ec):

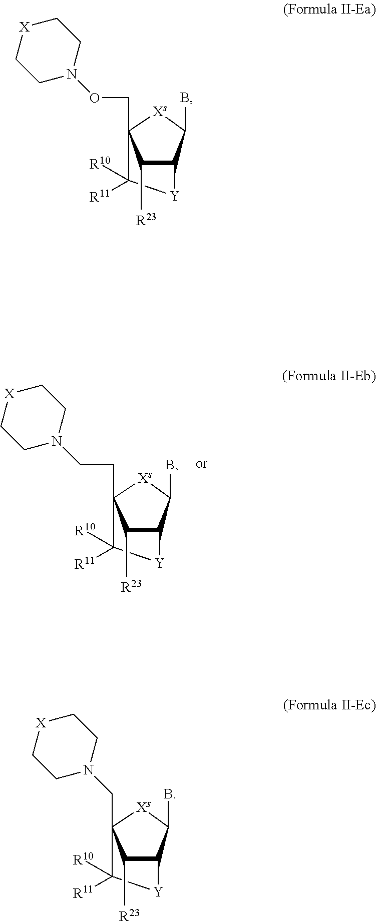

In some nucleosides of Formula (II-Ea), (II-E¹), (II-E²), (II-Eb) and/or (II-Ec), R²³ is a bond to an internucleotide linkage to a subsequent nucleoside; and X^{S}, B, Y, R¹⁰ and R¹¹ are as defined for Formula (II).

In some embodiments of any one of the aspects described herein, the nucleoside of Formula (II-Ea) is of Formula (II-Ed) or (II-Ee):

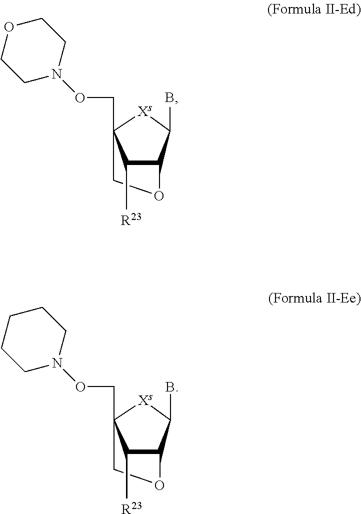

In some embodiments of any one of the aspects described herein, the nucleoside of Formula (II-E) is of Formula (II-E³) or (II-E⁴):

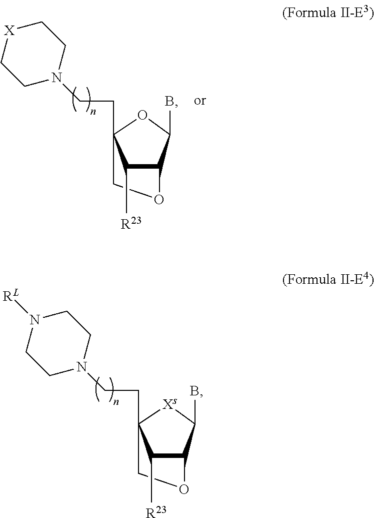

where n is 0 or an integer selected from 1 to 30 (e.g., n is 1, 2, 3, 4, 5, 6, 7, 8, 9, 10, 11, 12, 13, 14, 15, 16, 16, 17, 18, 19, 20, 21, 22, 23, 24, 25, 26, 27, 28, 29 or 30, such as n is 1, 2, 3, 4, 5 or 6, preferably n is 0 or 1); and
R^{L} is H, a ligand, a linker covalently bonded to one or more ligands, aliphatic and aromatic alkyl, alkylester, alkylamine, dimethylamino alkyl, alkylether, alkylthioether, heteroaromatic alkyl, allyl, vinyl, alkyl groups functionalized with disulfide, oxime, ketone, acetal, hemiacetal, cleavable peptides, or cleavable sugars.

In some embodiments of any one of the aspects described herein, the nucleoside of Formula (II-Eb) is of Formula (II-Ef):

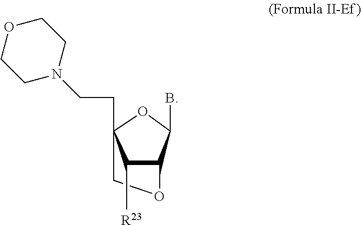

In some embodiments of any one of the aspects described herein, the nucleoside of Formula (II-Ec) is of Formula (II-Eg):

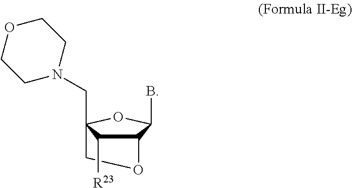

In some embodiments of the various aspects described herein, R⁵ is not morpholin-4-yl unless R²⁴ and R₂₂ taken together are 4′-C(R¹⁰R¹¹)ᵥ—Y-2′ or 4′-Y—C(R¹⁰R¹¹)ᵥ-2′.

In some embodiments of the various aspects described herein, when R²² is H, hydroxyl, protected hydroxyl, alkoxy, or halogen; R²³ is a bond to a internucleotide linkage to a subsequent nucleoside; R²⁴ is H; and X^{S} is O, then R⁵ is not morpholin-4-yl.

In yet another aspect, provided herein is a double-stranded nucleic acid comprising a first strand and a second strand complementary to the first strand, and wherein at least one of the first and second strand is an oligonucleotide comprising a nucleoside of Formula (II) described herein.

In some embodiments of the various aspects described herein, the double-stranded nucleic acid comprises a first strand and a second strand complementary to the first strand, wherein one of the first stand and second strand is an oligonucleotide comprising a nucleoside of Formula (II) described herein, and the other strand comprises on its 5′-end a vinylphosphonate group (VP) group (e.g., *═CH—X^{P}, X^{P} is a phosphate group and * is C5′), C₃₋₆ cycloalkylphosphonate (e.g., cyclopropylphosphonate), monophosphate ((HO)₂(O) P—O-5′), diphosphate ((HO)₂(O) P—O—P(HO)(O)—O-5′), triphosphate ((HO)₂(O) P—O—(HO)(O) P—O—P(HO)(O)—O-5′); monothiophosphate (phosphorothioate, (HO)2(S) P—O-5′), monodithiophosphate (phosphorodithioate; (HO)(HS)(S) P—O-5′), phosphorothiolate ((HO)2(O) P—S-5′); alpha-thiotriphosphate; beta-thiotriphosphate; gamma-thiotriphosphate; phosphoramidates ((HO)₂(O) P—NH-5′, (HO)(NH₂)(O) P—O-5′), alkylphosphonates [(R^{P})(OH)(O) P—O-5′, R^{P} is optionally substituted C₁₋₃₀ alkyl, e.g., methyl, ethyl, isopropyl, or propyl)], alkyletherphosphonates [(R^{P1})(OH)(O) P—O-5′, R^{P1} is alkoxyalkyl, e.g., methoxymethyl (CH₂OMe) or ethoxymethyl], (HO)₂(X) P—O[—(CH₂)ₐ—O—P(X)(OH)—O]_{b}-5′ or (HO)₂(X) P—O[—(CH₂)ₐ—P(X)(OH)—O]_{b}-5′ or (HO)₂(X) P—[—(CH₂)ₐ—O—P(X)(OH)—O]_{b}-5′, or optionally substituted alkyl, and dialkyl terminal phosphates and phosphate mimics (e.g., HO[—(CH₂)ₐ—O—P(X)(OH)—O]_{b}-5′, H₂N[—(CH₂)ₐ—O—P(X)(OH)—O]-5′, H[—(CH₂)ₐ—O—P(X)(OH)—O]_{b}-5′, Me₂N[—(CH₂)ₐ—O—P(X)(OH)—O]_{b}-5′, HO[—(CH₂)ₐ—P(X)(OH)—O]_{b}-5′, H₂N[—(CH₂)ₐ—P(X)(OH)—O]_{b}-5′, H[—(CH₂)ₐ—P(X)(OH)—O]_{b}-5′, Me₂N[—(CH₂)ₐ—P(X)(OH)—O]_{b}-5′, wherein X is O or S; and a and b are each independently 1-10. For example, the double-stranded nucleic acid comprises a first strand and a second strand complementary to the first strand, wherein one of the first stand and second strand is an oligonucleotide comprising a nucleoside of Formula (II) described herein, and the other strand comprises on its 5′-end a vinylphosphonate group, e.g., an E-vinylphosphonate group.

In some embodiments of any one of the aspects described herein, a nuclease resistant modification is a nucleoside of Formula (II).

In some embodiments of any one of the aspects described herein, an oligonucleotide described herein comprises at least one, e.g., 2, 3, 4, 5, 6, 7, 8, 9, 10 or more phosphorothioate internucleoside linkages. For example, the oligonucleotide comprises at least 4 phosphorothioate internucleoside linkages, such as at least 6 phosphorothioate internucleoside linkages or at least 8 phosphorothioate internucleoside linkages.

In some embodiments of any one of the aspects described herein, the dsRNA comprises at least one, e.g., 2, 3, 4, 5, 6, 7, 8, 9, 10 or more phosphorothioate internucleoside linkages. For example, the dsRNA comprises at least 4 phosphorothioate internucleoside linkages, such as at least 6 phosphorothioate internucleoside linkages or at least 8 phosphorothioate internucleoside linkages.

It is noted that the phosphorothioate internucleoside linkages can be present in one strand or both strands. Further, the phosphorothioate internucleoside linkages can be present anywhere in the strand. For example, the phosphorothioate internucleoside linkages can be present at one end of the strand, at both ends of the strand, both at one end and at internal positions of the strand, or at both ends and at internal positions of the strand. Preferably, the phosphorothioate internucleoside linkages are present at both ends of the strand.

In some embodiments, the antisense strand comprises at least one, e.g., two, three, four or more phosphorothioate internucleoside linkages. For example, the antisense strand comprises 4 or more phosphorothioate internucleoside linkages. In some embodiments of any one of the aspects described herein, the antisense strand comprises a phosphorothioate internucleoside linkage between positions 1 and 2, counting from the 3′-end of the strand, and a phosphorothioate internucleoside linkage between positions 1 and 2, counting from the 5′-end of the strand. In yet some other embodiments of any one of the aspects described herein, the antisense strand comprises a phosphorothioate internucleoside linkage between positions 1 and 2, and between positions 2 and 3, counting from the 3′-end of the strand, and a phosphorothioate internucleoside linkage between positions 1 and 2, counting from the 5′-end of the strand. In still some other embodiments of any one of the aspects described herein, the antisense strand comprises a phosphorothioate internucleoside linkage between positions 1 and 2, and between positions 2 and 3, counting from the 3′-end of the strand, and a phosphorothioate internucleoside linkage between positions 1 and 2, and between positions 2 and 3, counting from the 5′-end of the strand. In yet still some other embodiments of any one of the aspects described herein, the antisense strand comprises a phosphorothioate internucleoside linkage between positions 1 and 2, between positions 2 and 3, and between positions 3 and 4, counting from the 3′-end of the strand, and a phosphorothioate internucleoside linkage between positions 1 and 2, counting from the 5′-end of the strand. In some embodiments of any one of the aspects described herein, the antisense strand comprises a phosphorothioate internucleoside linkage between positions 1 and 2, counting from the 3′-end of the strand, and a phosphorothioate internucleoside linkage between positions 1 and 2, and between positions 2 and 3, counting from the 5′-end of the strand. In yet other embodiments of any one of the aspects described herein, the antisense strand comprises a phosphorothioate internucleoside linkage between positions 1 and 2, counting from the 3′-end of the strand, and a phosphorothioate internucleoside linkage between positions 1 and 2, between positions 2 and 3, and between positions 3 and 4, counting from the 5′-end of the strand.

Like the antisense strand, the sense strand can also comprise one or more, e.g., two, three, four or more phosphorothioate internucleoside linkages. For example, the sense strand comprises a phosphorothioate internucleoside linkage between positions 1 and 2, counting from 5′-end of the strand. In some embodiments of any one of the aspects described herein, the sense strand comprises a phosphorothioate internucleoside linkage between positions 1 and 2, counting from 5′-end of the strand, and between positions 1 and 2, counting from 3′-end of the strand.

In yet some embodiments of any one of the aspects described herein, the sense strand comprises a phosphorothioate internucleoside linkage between positions 1 and 2, and between positions 2 and 3, counting from 5′-end of the strand. For example, the sense strand comprises a phosphorothioate internucleoside linkage between positions 1 and 2, and between positions 2 and 3, counting from 5′-end of the strand, and between positions 1 and 2, and between positions 2 and 3, counting from 3′-end of the strand.

In some embodiments of any one of the aspects described herein, the antisense and the sense strand can be independently at least about 18, e.g., about 19, about 20, about 21, about 22, about 23, about 24, about 25, about 26, about 27, about 28, about 29, about 30 or more, nucleotides in length. For example, the antisense strand is about 20, about 21, about 22, about 23, about 24, about 25 or about 26 nucleotides in length. In some embodiments of any one of the aspects described herein, the antisense strand is about 22, about 23 or about 25 nucleotides in length.

Similar to the antisense strand, in some embodiments of any one of the aspects described herein, the sense strand is at least about 16, e.g., about 17, about 18, about 19, about 20, about 21, about 22, about 23, about 24, about 25, about 26, or more, nucleotides in length. For example, the sense strand is about 19, about 20, about 21, about 22, about 23, about 24 or about 25 nucleotides in length. In some embodiments of any one of the aspects described herein, the sense strand is about 21 nucleotides in length.

In some embodiments of any one of the aspects described herein, the antisense strand is 22, 23 or 25 nucleotides in length and the sense strand is 21 nucleotides in length.

In some embodiments of any one of the aspects described herein, the sense strand is 15 nucleotides in length and the antisense strand is 18, 19, 20, 21, or 22 (e.g., 20) nucleotides in length. In some embodiments of any one of the aspects described herein, the sense strand is 19 nucleotides in length and the antisense strand is 19, 20, or 21 nucleotides in length. In some embodiments of any one of the aspects described herein, the sense strand is 20 nucleotides in length and the antisense strand is 20, 21, or 22 nucleotides in length. In some embodiments of any one of the aspects described herein, the sense strand is 21 nucleotides in length and the antisense strand is 21, 22, or 23 nucleotides in length. In some embodiments of any one of the aspects described herein, the sense strand is 20-24 (e.g., 22) nucleotides in length and the antisense strand is 34-38 (e.g. 36) nucleotides in length.

In some embodiments of the various aspects described herein, the double-stranded region of the dsRNA can be at least about 18, e.g., about 19, about 20, about 21, about 22, about 23, about 24, about 25 or more base-pairs, for example, a double-stranded region of about 21 base-pairs.

In some embodiments of any one of the aspects described herein, the antisense strand is about 21, about 22, about 23, about 24 or about 25 nucleotides in length, the sense strand is about 21 nucleotides in length, and the dsRNA comprises a double-stranded region of at least 18, e.g., 19, 20 or 21 base-pairs, such as 21 base-pairs.

Generally, a ligand is linked to the 3′-end of the antisense strand. The ligand can be linked to any available position of the nucleotide at the 3′-end, i.e., nucleotide at position 1 (counting 3′-end) of the antisense strand. For example, the ligand can be attached to the 3′-hydroxyl, 2′-hydroxyl (if present), or a position in the nucleobase. In some embodiments of any one of the aspects described herein, the ligand is linked to 3′-hydroxyl of the nucleotide at position 1, counting from 3′-end, of antisense strand. The ligand can be linked directly, i.e., via a bond, or by a linker to the 3′-end of the antisense strand.

It is noted that the ligand or the linker attached to the ligand can be linked to the 3′-end of the antisense strand via any modified or unmodified internucleoside linkage known and available in the art. For example, the ligand or the linker attached to the ligand can be linked to the 3′-end of the antisense strand via any negatively charged moiety. For example, the ligand or the linker attached to the ligand can be linked to the 3′-end of the antisense strand via a phosphodiester (PO), phosphorothioate (PS), phosphorodithioate (PS2), PN (e.g., RSO₂—N═P(OH) type or (HO) P—NHR or (HO) P—NR₂, each where R includes, but is not limited to, an aliphatic (e.g., C1-20 alkyl), cycloaliphatic, heterocyclic, aromatic, or heteroaromatic group, each of which may be optionally substituted; or where both R groups, together with the nitrogen to which they are attach form a 4-10 membered monocyclic heterocyclic or bicyclic heterocyclic group, the heterocyclic group optionally having 1 or 2 additional heteroatoms selected from O, N, and S, and where heterocyclic group is optionally substituted. Optional substituents can be one or more (e.g., 1, 2, or 3 groups) independently selected from the group consisting of halogen, cyano, nitro, azido, hydroxy, amino, carboxy, oxo (═O), thia (═S), imino (═N(H)), C₁₋₆alkylimino (═N(R)), C₁₋₆alkylamino (R(H) N—), diC₁₋₆alkylamino (R₂N—), C₁₋₆alkyl, C₁₋₆alkoxy, C₁₋₆acyl (RC(O)—), C₁₋₆alkylester (ROC(O)—), amido (H₂NC(O)—), C₁₋₆alkylamide (R(H) NC(O)—), diC₁₋₆alkylamide (R²NC(O)—), C₁₋₆ acylamino (RC(O)N(H)—) linkage.

In some embodiments of any one of the aspects described herein, the ligand or linker attached to the ligand is linked to the 3′-end of the antisense strand via a phosphorothioate internucleoside linkage.

Linker can be selected in order to position the ligand it away from the PAZ domain of Ago. Accordingly, in some embodiments of any one of the aspects described herein, the linker connecting the ligand to the 3′-end of the antisense strand is from about 5 Angstroms to about 250 Angstroms in length. For example, the linker connecting the ligand to the 3′-end of the antisense strand is from about 10 Angstroms to about 200 Angstroms length, e.g., from about 15 Angstroms to about 150 Angstroms, from about 20 Angstroms to about 100 Angstroms, from about 25 Angstroms to about 75 Angstroms, from about 5 Angstroms to about 50 Angstroms, from about 10 Angstroms to about 40 Angstroms or from about 20 Angstroms to about 30 Angstroms in length.

In some embodiments of any one of the aspects described herein, the linker has a chain length of at least 6 atoms. For example, the linker has a chain length of at least 7, 8, 9, 10, 11, 12, 13, 14, 15, 16, 17, 18, 19, 20, 21, 22, 23, 24, 25, 26, 27, 28, 29, 30 or more atoms). In some embodiments of any one of the aspects described herein, the linker has a chain length of 6, 7, 8, 9, 10, 11, 12, 13, 14, 15, 16, 17, 18, 19, 20, 21, 22, 23, 24, 25, 26, 27, 28, 29 or 30 atoms.

In some embodiments of any one of the aspects described herein, the ligand is linked to the 3′-end of the antisense strand via a linker. For example, the ligand is linked to the 3′-end of the antisense strand via a hydrophobic linker.

In embodiments of the various aspects described herein, the linker can comprise a carrier connected to a carrier. In some embodiments, the carrier comprises a hydrogen-bonding acceptor (e.g., a tertiary amide or tertiary amine). In some embodiments, the carrier comprises a pyrrolidine ring.

The inventors have discovered inter alia pharmacokinetic (PK)/pharmacodynamic (PD) properties of the dsRNAs comprising a ligand linked to the 3′-end of the antisense strand can be improved by including a second ligand in the dsRNAs. Accordingly, in some embodiments of any one of the aspects described herein, the dsRNA comprises a second ligand. The second ligand can be attached or linked to the sense strand or the antisense strand. Preferably, the second ligand is linked to the sense strand. In some embodiments of any one of the aspects described herein, the second ligand is linked to 3′-end of the sense strand. In some other embodiments of any one of the aspects described herein, the second ligand is linked to 5′-end of the sense strand. It is noted that the ligand linked to the antisense strand and second ligand can be same or different. Preferably, the ligand linked to the antisense strand and second ligand are different.

Embodiments of the various aspects described herein, include a ligand, such as a targeting ligand, a PK modulator, or an endosomolytic ligand. Accordingly, the ligand linked to the 3′-end of the antisense strand can be a targeting ligand, PK modulator or an endosomolytic ligand. Preferably, the ligand linked to the 3′-end of the antisense strand is a targeting ligand, e.g., mono- or multi-valent N-acetylgalactosamine (GalNac).

When present, the second ligand can be a targeting ligand, PK modulator or an endosomolytic ligand. For example, second ligand is a ligand capable of binding to a serum protein, e.g., serum albumin. Exemplary ligands capable of binding with serum albumin include, but are not limited to, iodipamide, azapropazone, indomethacin, tiblone (TIB), 3-carboxy-4-methyl-5-propyl-2-furanpropanoic acid (CMPF), DIS, oxyphenbutazone, phenylbutazone, warfarin, indoxyl sulfate, diflunisal, halothane, ibuprofen, and diazepam, propofol.

In some embodiments of any one of the aspects described herein, the ligand linked to the sense strand, i.e., the second ligand is a PK modulator.

In some embodiments of any one of the aspects described herein, the ligand linked to the sense strand, i.e., the second ligand is a mannose receptor ligand (e.g., multivalent mannose).

In some embodiments of any one of the aspects described herein, the ligand linked to the sense strand, i.e., the second ligand is a folic acid ligand.

In some embodiments of any one of the aspects described herein, the ligand linked to the 3′-end of the antisense strand is a targeting ligand, e.g., mono- or multi-valent GalNAc, and the second ligand is a PK modulator, e.g., ibuprofen.

In some embodiments of any one of the aspects described herein, the ligand linked to the 3′-end of the antisense strand is a targeting ligand, e.g., mono- or multi-valent GalNAc, and the ligand linked to the sense strand is a mannose receptor ligand (e.g., mannose).

In some embodiments of any one of the aspects described herein, the ligand linked to the 3′-end of the antisense strand is a targeting ligand, e.g., mono- or multi-valent GalNAc, and the ligand linked to the sense strand is a folic acid ligand.

In embodiments of any one of the aspects described herein, each ligand can be selected independently from the group consisting of peptides, centyrins, antibodies (e.g., antiCD-4 antibodies and antiCD-117 antibodies), antibody fragments, T-cell targeting ligands, B-cell targeting ligands, cancer cell targeting ligands (e.g., DUPA, folate, and RGD), spleen targeting functionalities, lung targeting functionalities, bone marrow targeting functionalities, phage display peptides, cell permeation peptides (CPPs), integrin ligands, multianionic ligands, multicationic ligands, monovalent and multivalent carbohydrates (e.g., GalNAc, mannose, mannose-6 phosphate, mucose, and mlucose), kidney targeting ligands, BBB penetration ligands, lipids, and amino acids (e.g., L-amino acids, D-amino acids, and β-amino acids).

It is noted that the double-stranded RNAs described can comprise one or more additional nucleic acid modifications such as nucleobase modifications, sugar modifications, inter-sugar linkage modifications, or any combination thereof. Accordingly, in some embodiments of any one of the aspects described herein, dsRNA comprises at least one, e.g., 2, 3, 4, 5, 6, 7, 8, 9, 10 or more 2′-fluoro nucleotides. For example, the antisense strand and/or the sense stand comprises independently at least one, e.g., 2, 3, 4, 5 or more 2′-fluoro nucleotides.

In some embodiments of any one of the aspects described herein, the antisense strand comprises a 2′-fluoro nucleotide at positions 2, 14 and 16, counting from the 5′-end of the antisense strand. For example, the antisense strand comprises a 2′-fluoro nucleotide at positions 2, 6, 14 and 16, counting from the 5′-end of the antisense strand. In another non-limiting example, the antisense strand comprises a 2′-fluoro nucleotide at positions 2, 6, 9, 14 and 16, counting from the 5′-end of the antisense strand. In some further examples, the antisense strand comprises a 2′-fluoro nucleotide at positions 2, 6, 8, 9, 14 and 16, counting from the 5′-end of the antisense strand.

In some embodiments of any one of the aspects described herein, the antisense strand comprises a 2′-fluoro nucleotide at positions 2, 5, 7, 12, 14 and 16 counting from the 5′-end of the antisense strand.

In some embodiments of any one of the aspects described herein, the sense strand comprises a 2′-fluoro nucleotide at positions 7, 9 and 11, counting from the 5′-end of the sense strand or at positions 11, 13 and 15, counting from the 3′-end of the sense strand. For example, the sense strand comprises a 2′-fluoro nucleotide at positions 7, 9, 10 and 11, counting from the 5′-end of the sense strand or at positions 11, 12, 13 and 15, counting from the 3′-end of the sense strand.

In some embodiments of any one of the aspects described herein, the sense strand comprises a 2′-fluoro nucleotide at positions 9, 10, and 11, counting from the 5′-end of the sense strand or at positions 11, 12, and 13 counting from the 3′-end of the sense strand.

In some embodiments of any one of the aspects described herein, the antisense strand comprises a 2′-fluoro nucleotide at least at positions 2, 14 and 16, counting from the 5′-end of the antisense strand, and the sense strand comprises a 2′-fluoro nucleotide at least at positions 7, 9 and 11, counting from the 5′-end of the sense strand or at least at positions 11, 13 and 15, counting from the 3′-end of the sense strand. For example, the antisense strand comprises a 2′-fluoro nucleotide at least at positions 2, 6, 14 and 16, counting from the 5′-end of the antisense strand, and the sense strand comprises a 2′-fluoro nucleotide at least at positions 7, 9 and 11, counting from the 5′-end of the sense strand or at least at positions 11, 13 and 15, counting from the 3′-end of the sense strand. In another example, the antisense strand comprises a 2′-fluoro nucleotide at least at positions 2, 6, 9, 14 and 16, counting from the 5′-end of the antisense strand, and the sense strand comprises a 2′-fluoro nucleotide at least at positions 7, 9 and 11, counting from the 5′-end of the sense strand or at least at positions 11, 13 and 15, counting from the 3′-end of the sense strand. In yet another example, the antisense strand comprises a 2′-fluoro nucleotide at least at positions 2, 6, 8, 9, 14 and 16, counting from the 5′-end of the antisense strand, and the sense strand comprises a 2′-fluoro nucleotide at least at positions 7, 9 and 11, counting from the 5′-end of the sense strand or at least at positions 11, 13 and 15, counting from the 3′-end of the sense strand.

In some further non-limiting examples, the antisense strand comprises a 2′-fluoro nucleotide at least at positions 2, 14 and 16, counting from the 5′-end of the antisense strand, and the sense strand comprises a 2′-fluoro nucleotide at least at positions 7, 9 and 11, counting from the 5′-end of the sense strand or at least at positions 11, 12, 13 and 15, counting from the 3′-end of the sense strand. For example, the antisense strand comprises a 2′-fluoro nucleotide at least at positions 2, 6, 14 and 16, counting from the 5′-end of the antisense strand, and the sense strand comprises a 2′-fluoro nucleotide at least at positions 7, 9, 10 and 11, counting from the 5′-end of the sense strand or at least at positions 11, 12, 13 and 15, counting from the 3′-end of the sense strand. In another example, the antisense strand comprises a 2′-fluoro nucleotide at least at positions 2, 6, 9, 14 and 16, counting from the 5′-end of the antisense strand, and the sense strand comprises a 2′-fluoro nucleotide at least at positions 7, 9, 10 and 11, counting from the 5′-end of the sense strand or at least at positions 11, 12, 13 and 15, counting from the 3′-end of the sense strand. In yet another example, the antisense strand comprises a 2′-fluoro nucleotide at least at positions 2, 6, 8, 9, 14 and 16, counting from the 5′-end of the antisense strand, and the sense strand comprises a 2′-fluoro nucleotide at least at positions 7, 9, 10 and 11, counting from the 5′-end of the sense strand or at least at positions 11, 12, 13 and 15, counting from the 3′-end of the sense strand.

The dsRNAs described herein can comprise one or more, e.g., 2, 3, 4, 5, 6, 7, 8, 9, 10 or more 2′-deoxy (i.e., 2′-H or DNA) nucleotides. For example, the antisense strand and/or the sense stand comprises independently at least one, e.g., 2, 3, 4, 5 or more 2′-deoxy (i.e., 2′-H or DNA) nucleotides.

In some embodiments of any one of the aspects described herein, the antisense strand comprises a DNA nucleotide at positions 2, 5, 7, and 12 counting from the 5′-end of the antisense strand. In some embodiments of any one of the aspects described herein, the antisense strand comprises a DNA nucleotide at positions 2, 5, 7, 12, and 14 counting from the 5′-end of the antisense strand. In some embodiments of any one of the aspects described herein, the antisense strand comprises a DNA nucleotide at positions 2, 5, 7, 12, 14 and 16 counting from the 5′-end of the antisense strand.

In some embodiments of any one of the aspects described herein, the antisense strand comprises a DNA nucleotide at positions 2, 5, 7 and 12, counting from the 5′-end of the antisense strand; and a 2′-fluoro nucleotide at position 14 of the antisense strand.

The dsRNAs described herein can comprise one or more, e.g., 2, 3, 4, 5, 6, 7, 8, 9, 10 or more 2′-OMe nucleotides. For example, the antisense strand and/or the sense stand comprises independently at least one, e.g., 2, 3, 4, 5 or more 2′-OMe nucleotides. In some embodiments of any one of the aspects described herein, all remaining nucleotides, i.e., other than modifications specified herein, in the antisense strand are 2′-OMe nucleotides. Similarly, in some embodiments of any one of the aspects described herein, all remaining nucleotides, i.e., other than modifications specified herein, in the antisense strand are 2′-OMe nucleotides.

In some embodiments of any one of the aspects described herein, the antisense strand comprises a phosphate group or a phosphate analog or derivative thereof at its 5′-end. For example, the antisense strand comprises a 5′-vinylphosphonate nucleotide at its 5′-end. For example, the antisense strand comprises a 5′-E-vinylphosphanate nucleotide at its 5′-end.

In some embodiments of any one of the aspects described herein, the dsRNA comprises at least one, e.g., 2, 3, 4, 5, 6, 7, 8, 9, 10 or more locked nucleic acid (LNA) or bridged nucleic acid (BNA) nucleotides. For example, the antisense and/or the sense strand comprises independently at least one, e.g., 2, 3, 4, 5 or more LNA or BNA nucleotides.

In some embodiments of any one of the aspects described herein, the dsRNA comprises at least one, e.g., 2, 3, 4, 5, 6, 7, 8, 9, 10 or more cyclohexene nucleic acid (CeNA) nucleotides. For example, the antisense and/or the sense strand comprises independently at least one, e.g., 2, 3, 4, 5 or more CeNA nucleotides.

In some embodiments of any one of the aspects described herein, the dsRNA comprises at least one, e.g., 2, 3, 4, 5, 6, 7, 8, 9, 10 or more thermally stabilizing modification. For example, the antisense and/or the sense strand comprises independently at least one, e.g., 2, 3, 4, 5 or more thermally stabilizing modification.

In some embodiments of any one of the aspects described herein, the dsRNA comprises at least one, e.g., 2, 3, 4, 5, 6, 7, 8, 9, 10 or more abasic nucleotides. For example, the antisense and/or the sense strand comprises independently at least one, e.g., 2, 3, 4, 5 or more abasic nucleotides.

In some embodiments of any one of the aspects described herein, the dsRNA comprises at least one, e.g., 2, 3, 4, 5, 6, 7, 8, 9, 10 or more 2′-deoxy nucleotides. For example, the antisense and/or the sense strand comprises independently at least one, e.g., 2, 3, 4, 5 or more 2′-deoxy nucleotides. In some embodiments, the antisense strand comprises one or more, e.g., one, two or more 2′-deoxy nucleotides in the single-stranded overhang.

In some embodiments of any one of the aspects described herein, the dsRNA comprises at least one, e.g., 2, 3, 4, 5, 6, 7, 8, 9, 10 or acyclic (e.g., unlocked nucleic acid (UNA), glycol nucleic acid (GNA) or(S)-glycol nucleic acid (S-GNA)) nucleotides. For example, the antisense and/or the sense strand comprises independently at least one, e.g., 2, 3, 4, 5 or more UNA and/or GNA nucleotides.

In some embodiments of any one of the aspects described herein, the dsRNA comprises at least one, e.g., 2, 3, 4, 5, 6, 7, 8, 9, 10 or thermally destabilizing modifications. For example, the antisense and/or the sense strand comprises independently at least one, e.g., 2, 3, 4, 5 or more thermally destabilizing modifications. Some exemplary thermally destabilizing modifications include, but are not limited to, abasic nucleotides, 2′-deoxy nucleotides, acyclic nucleotides (e.g., UNA, GNA and(S)-GNA), 2′-5′ linked nucleotides (3′-RNA), threose nucleotides (TNA), 2′ gem Me/F nucleotides, and a mismatch with the opposing nucleotide in the other strand.

In some embodiments of any one of the aspects described herein, the antisense strand comprises at least one thermally destabilizing modification in the seed region (i.e., positions 2-9 from the 5′-end) of the antisense strand. For example, the antisense strand comprises a thermally destabilizing modification at least at one of positions 6, 7 or 8, counting from the 5′-end of the strand. In some embodiments of any one of the aspects described herein, the antisense strand comprises a thermally destabilizing modification at position 7, counting from the 5′-end of the strand.

The double-stranded nucleic acid can comprise blunt ends and/or single-stranded overhangs at the end. For example, the double-stranded nucleic acid can comprise comprises a blunt end at 5′-end of the antisense strand. In another example, the double-stranded nucleic acid can comprise comprises a 1-5 nucleotide single-stranded overhang at 3′-end of the antisense strand, e.g., the 3′-end of the antisense strand extends past the 5′-end of the sense strand.

In another aspect, provided herein is a pharmaceutical composition comprising an oligonucleotide or dsRNA molecule described herein alone or in combination with a pharmaceutically acceptable carrier or excipient.

In yet another aspect, provided herein is a cell comprising an oligonucleotide or dsRNA molecule described herein.

In still another aspect, provided herein is a gene silencing kit comprising an oligonucleotide or dsRNA molecule described herein.

Also, provided herein is a method for silencing a target gene, in a cell. The method comprises a step of introducing: (i) a dsRNA molecule described herein into the cell, where one of the strands, e.g., the antisense of the dsRNA comprises a nucleotide sequence substantially complementary to a nucleotide sequence of the target gene; and/or (ii) an oligonucleotide described herein, wherein the oligonucleotide comprises a nucleotide sequence substantially complementary to a nucleotide sequence of the target gene.

In another aspect, provided herein is a method for inhibiting or reducing the expression of a target gene in a subject. The method comprises administering to the subject: (i) a dsRNA molecule described herein, where one of the strands, e.g., the antisense of the dsRNA comprises a nucleotide sequence substantially complementary to a nucleotide sequence of the target gene; and/or (ii) an oligonucleotide described herein, wherein the oligonucleotide comprises a nucleotide sequence substantially complementary to a nucleotide sequence of the target gene.

### BRIEF DESCRIPTION OF THE DRAWINGS

FIGS. 1A and 1B are schematics of siRNAs with GalNAc conjugated at the 3′ end of sense strand (FIG. 1A) and the 3′ end of antisense strand (FIG. 1B). Deoxythymine residues are indicated by blue, 2′-fluoro is indicated in green, 2′-O-methyl in black. Phosphorothioate linkages are indicated by orange lines.

FIG. 1C depicts the chemical structure of the GalNAc ligand.

FIG. 2 shows ASGPR binding affinities of the conjugated GalNAc moieties to siRNA.

FIGS. 3A-3C are graphs showing in vitro activity of siRNAs targeting mTTR (FIG. 3A), C5 (FIG. 3B) and FXII (FIG. 3C) by transfection (upper panels) or free uptake (lower panels).

FIG. 4 is a bar graph showing siRNA with GalNAc conjugated to the 3′ end of the antisense strand is effective in vivo. Percentage of TTR protein in circulation after treatment of mice with GalNAc conjugates I or II relative to PBS-treated control mice (n=3). Blood samples were drawn 4 days (violet) and 7 days (green) post-dose, and TTR protein levels were quantified by ELISA. Serum TTR protein levels from individual animals were normalized to PBS-treated control group and are expressed as the mean±standard deviation.

FIG. 5 shows relative TTR protein in circulation after treatment with GalNAc conjugates II and VII relative to pre-dose levels. The siRNAs are shown schematically above the graph. Locations of phosphorothioate linkages are indicated by orange lines. C57BL/6 mice were treated subcutaneously with 1 mg/kg II (blue) and 0.5 (green), 1 (red), and 2.5 (brown) mg/kg VII (n=3). Blood samples were drawn 4, 7, 10, 14, and 21 days post-dose, and TTR was quantified by ELISA. Serum TTR protein levels from individual animals were normalized to pre-dose level and are expressed as the mean±standard deviation.

FIGS. 6A and 6B are graphs showing percentage of TTR protein in circulation after treatment with GalNAc conjugates I (blue), II (orange), and VIII (violet) compared to pre-dose levels in C57BL/6 mice (n=3). The siRNAs are shown schematically to the right. Mice were treated subcutaneously with (FIG. 6A) 2.5 mg/kg and (FIG. 6B) 1 mg/kg. Blood samples were drawn 7, 14, 21, and 28 days post-dose, and TTR protein was quantified by ELISA. Serum TTR protein levels from individual animals were normalized to pre-dose level and are expressed as the mean±standard deviation.

FIGS. 7B and 7C are graphs showing percentage of TTR protein in circulation after treatment with GalNAc conjugates II (blue), IX (orange), and X (violet) compared to pre-dose levels in C57BL/6 mice (n=3). Mice were treated subcutaneously with (FIG. 7A) 2.5 mg/kg or (FIG. 7B) 1.0 mg/kg. Blood samples were drawn 7, 14, 21, and 28 day post-dose, and TTR protein was quantified by ELISA. Serum TTR protein levels from individual animals were normalized to pre-dose level and are expressed as the mean±standard error.

FIG. 8A shows liver siRNA levels after single subcutaneous administration of 1 mg/kg siRNA in wild-type C57BL/6 mice. Livers were collected five days post-dose and siRNA levels were quantified by RT-qPCR. Sense strand (blue) and antisense strand (red) levels were evaluated. Data are expressed as the mean+/−standard deviation.

FIG. 8B depicts Ago2-loaded siRNA levels after single subcutaneous administration of 1 mg/kg siRNA in wild-type C57BL/6 mice. Livers were collected five days post-dose and siRNA levels in Ago2 were quantified by RT-qPCR. Sense strand (blue) and antisense strand (red) levels were evaluated. Data are expressed as the mean+/−standard deviation.

FIG. 8C is a comparison of mTTR mRNA knockdown in liver after single SC administration of siRNA I, VIII, II and VII in wild-type C57BL/6 mice; results are presented as % mTTR mRNA remaining in liver after single dose SC administration at 1 mg/kg.

FIGS. 9A and 9B show that duplex (XIV) shows better efficacy and longer duration of action compared to (II), (XIII), (XI) and (XII) after 42 day; (FIG. 9A) 2.5 mg/kg (FIG. 9B) 1.0 mg/kg. Chemistry used: 3′-GalNAc in antisense strand, 8PS for II and XI-XIV.

FIG. 10 is a bar-graph showing relative TTR protein in circulation after treatment with GalNAc conjugates XV and XVI relative to pre-dose levels. The siRNAs are shown schematically above the graph. Locations of phosphorothioate linkages are indicated by orange lines. C57BL/6 mice were treated subcutaneously with 1 mg/kg XV (blue) and 0.5 (green), 1 (red), and 2.5 (brown) mg/kg XVI (n=3). Blood samples were drawn 4, 7, 10, 14, and 21 days post-dose, and TTR was quantified by ELISA. Serum TTR protein levels from individual animals were normalized to pre-dose level and are expressed as the mean±standard deviation.

FIG. 11A depicts in vivo gene silencing of 3′-AS GalNAc (6PS and 8PS) conjugates of C5 siRNAs: PD observed on Day 5. Comparison of gene silencing of conjugates III, IV and XVII in wild-type C57BL/6 mice (n=3); results are presented as % C5 protein remaining in circulation after single dose SC administration at 1 mg/kg dose, relative to pre dose. Blood samples were drawn 5 day post-dose for C5 protein evaluation by ELISA. Serum C5 protein levels from individual animals were normalized to pre-dose level and are expressed as the mean±standard error. Chemistry used in FIGS. 11A-11C: Parent 3′-GalNAc in sense strand (parent control), 6PS for III; 3′-GalNAc in antisense strand, 8PS for IV; and 3′-GalNAc in antisense strand, 6PS for XVII.

FIG. 11B shows tissue levels of siRNA-GalNAc conjugates in liver at 24 h after a single 1 mg/kg subcutaneous dose (SC, SD) in C57BL/6 mice (n=3). siRNA levels were determined using a PCR-based assay¹⁵.

FIG. 11C shows in vivo Ago2 loading of siRNA.

FIG. 12 shows docking of GalNAc linker attached to 3′-end of siRNA guide strand interacting with PAZ domain. The linker is positioned away from PAZ domain, which explains how GalNAc is accommodated in PAZ domain, hydroxyproline is stabilized within PAZ domain

FIG. 13A-13C show results of in vitro experiments (FIG. 13A) and in vivo study (FIGS. 13B and 13C). Both single stranded siRNA XIX and XX were dosed to wild type C57BL/6 mice for mouse transthyretin mRNA (mTTR) through single subcutaneous administration at 3.0 (FIG. 13B) and at 10.0 mg/kg (FIG. 13C) to observe dose response. Levels of circulating mTTR protein were analyzed after 3, 7, and 14-day post-dose.

FIG. 14 is a schematic representation of the design of an exemplary chemically modified siRNA.

FIG. 15 depicts some exemplary chemically modified siRNAs with GalNAc conjugated at 3′-end of antisense and sense strands.

FIGS. 16A and 16B are graphs showing the evaluation architecture shows (XIV) as the best construct at 2.5 mg/kg (FIG. 16A) and at 1.0 mg/kg (FIG. 16B).

FIGS. 17A and 17B depict activity single-stranded siRNAs. Both single strands siRNA XIX and XX were dosed to wild type C57BL/6 mice for mouse transthyretin mRNA (mTTR) through single subcutaneous administration at 3.0 (FIG. 17A) and at 10.0 mg/kg (FIG. 17B) to observe dose response. Levels of circulating mTTR protein were analyzed after 3, 7, and 14-day post-dose.

FIG. 18 depicts experiment design for evaluating the impact of ibuprofen ligands on conjugate efficacy and PK.

FIG. 19 depicts in vivo activity of GalNAc-Ibuprofen conjugates in wild-type mice. SEQ ID NOs are shown in Table 19.

FIG. 20 depicts the binding of ibuprofen conjugated siRNAs to human serum albumin (HAS). SEQ ID NOs are shown in Table 19.

FIG. 21 shows the PK/PD analysis of GalNAc vs GalNAc-Ibuprofen conjugate carrying hydrophobic PK-enhancers (albumin binding-ibuprofen).

FIG. 22 depicts different oligonucleotide sequences.

FIG. 23 shows in vivo TTR protein levels in serum samples over a 42-day period after subcutaneous (sc) administration of siRNAs with different ligands on the 5′ and 3′ end.

FIG. 24 shows in vivo TTR protein level in serum samples over a 42-day period after intravenous (iv) administration of siRNAs with different ligands on the 5′ and 3′ end.

FIGS. 25A-25E depict exemplary ligands. FIG. 25A shows different examples of ligands and representative L groups. FIG. 25B shows some exemplary ligand aldehydes. FIG. 25C shows some exemplary ligand acids. FIG. 25D shows some exemplary multivalent mannose based ligands, including acid for tri- and hexavalent mannose, aldehyde for tri- and hexavalent mannose, acid for hexa- and multivalent mannose, and aldehyde for hexa- and multivalent mannose. FIG. 25E shows multivalent mannose based ligands, including acid for hexa- and multivalent mannose, aldehyde for hexa- and multivalent mannose, acid for multivalent mannose, and aldehyde for multivalent mannose.

FIG. 26 shows different sense(S) and antisense (AS) strands with different ligands on the 5′ and 3′ end.

FIGS. 27-32 depict some exemplary embodiments of the disclosure.

FIG. 33 depicts some exemplary embodiments of the disclosure.

FIG. 34 is a bar graph showing modification of the sense strand with a phosphorylation blocker enhances silencing in mice. Mice (n=3 per group) were treated with a single dose (3 mg kg-1) of siRNA I, III, IV, or V targeting Apob (Table 10). The levels of circulating Apob protein were quantified at 3, 7, 14, and 21 days. Levels were normalized to Gapdh, and data are expressed as percent of Apob in the PBS-treated control animals.

FIG. 35 is a bar graph showing modification of antisense strands with Mo1 or Mo2 inhibits silencing. Mice (n=3 per group) were treated with a single dose (3 mg kg-1) of siRNA II, VI, VII and VIII targeting Apob (Table 10). The levels of circulating Apob protein were quantified at 3, 7, 14, and 21 days. Apob levels were normalized to Gapdh. Data are expressed as percent of Apob in the PBS-treated control animals.

FIG. 36 is a line graph showing gene silencing activity is inhibited by Mo2 modification of the antisense strand. Percent luciferase expression in TTR reporter assay as a function of siRNA concentration. The antisense strand of the siRNA targeting TTR was modified with the indicated morpholino analog. The parent strand did not have a 5′-modification.

FIG. 37 is a bar graph showing extended morpholino modifications at the 5′ position inhibits RISC loading. Total antisense RNA bound to recombinant human Ago2 quantified by stem-loop RT-PCR.

FIGS. 38A-38H are schematic representations showing morpholino analogues disrupt interaction of the 5′ phosphate with the MID domain of Ago2. FIGS. 12A-12D depict models of Ago2 bound to strands with (FIG. 38A) Mo1,20 (FIG. 38B) Mo2, (FIG. 38C) Pip, and (FIG. 38D) Mo3. FIG. 38E depicts overlay of the complexes shown in panels FIGS. 138A-38D. FIG. 38F depicts potential hydrogen-bond formation with Mo2. FIG. 38G depicts Ago2 surface coloured according to Coulombic potential (blue positive, white neutral). FIG. 38H depicts Ago2 surface coloured according to hydrophobicity (green lowest and pink highest).

FIG. 39 is a bar graph showing 5′-morpholino modified sense, antisense and control strand selection in vivo. Mice (n=3 per group) were treated with a single dose (3 mg kg-1) of parent siRNA and, duplexes I-VIII targeting Apob (Table 10). The levels of circulating Apob protein were quantified at 3, 7, 14 and 21 days. Apob levels were normalized to Gapdh mRNA. Data are expressed as percent of Apob in the PBS-treated control animals.

FIG. 40 is a schematic representation of duplexes that target mTTR.

FIG. 41 is a schematic representation of duplexes that target F9.

FIG. 42 is a schematic representation of duplexes that target ApoB.

FIG. 43 is a schematic representation of control LNA duplexes against mTTR. SEQ ID NOs are shown in Table 19.

FIGS. 44A-44G show IC₅₀ curves in PMH via transfection of duplexes that target mTTR: AD-57727 (FIG. 44A), AD-68895 (FIG. 44B), AD-617745 (FIG. 44C), AD-617746 (FIG. 44D), AD-617747 (FIG. 44E), AD-617748 (FIG. 44F), and AD-617749 (FIG. 44G).

FIG. 45 is a line-graph showing the timecourse of serum mTTR levels relative to pre dose after administration of exemplary duplexes targeting mTTR at 1 mg/kg dose. Sense strand of the duplex comprises LNA-Morpholinos at position 1 (S1) with variation on PS number and location, and modification of the antisense strand with 5′-vinylphosphate (5′-VP).

FIG. 46 is a bargrpah showing mTTR gene expression fold change in the mouse liver 28 days after administration of exemplary duplexes targeting mTTR at 1 mg/kg dose. Sense strand of the duplex comprises LNA-Morpholinos at position 1 (S1) with variation on PS number and location, and modification of the antisense strand with 5′-VP.

FIG. 47 a line-graph showing the timecourse of serum mTTR levels relative to pre dose after administration of control duplexes targeting mTTR at 1 mg/kg dose. Sense strand of the duplex comprises LNA at position 1 (S1) with variation on PS number and location, and modification of the antisense strand with 5′-VP.

### DETAILED DESCRIPTION

It is to be understood that both the foregoing general description and the following detailed description are exemplary and explanatory only and are not restrictive of the invention, as claimed. Herein, the use of the singular includes the plural unless specifically stated otherwise. As used herein, the use of “or” means “and/or” unless stated otherwise. Furthermore, the use of the term “including” as well as other forms, such as “includes” and “included”, is not limiting. Also, terms such as “element” or “component” encompass both elements and components comprising one unit and elements and components that comprise more than one subunit, unless specifically stated otherwise.

The section headings used herein are for organizational purposes only and are not to be construed as limiting the subject matter described. All documents, or portions of documents, cited in this application, including, but not limited to, patents, patent applications, articles, books, and treatises, are hereby expressly incorporated by reference in their entirety for any purpose.

### X^{S}

In some embodiments of the various aspects described herein, X^{S} can be O, CH₂, S, or NH. For example, X^{S} can be O or CH₂. In some preferred embodiments of any one of the aspects described herein, X^{S} is O.

### R⁵

In some embodiments of the various aspects describe herein, R⁵ is -L¹-R^{H} or —O—N(R¹³)R¹⁴, where L¹ is a bond, -L³-, C₁₋₃₀alkylene, C₂₋₃₀alkenylene, C₂₋₃₀alkynylene, *-L³-C₁₋₃₀alkylene *-L³-C₂₋₃₀alkenylene, or *-L³-C₂₋₃₀alkynylene; L³ is —O—, —N(R^{L3})—, —S—, —C(O)—, —S(O)—, —S(O)₂—, —P(X^{L3})(Y^{L3}R^{L3B})—; R^{L3} is hydrogen, optionally substituted C₁₋₃₀alkyl, optionally substituted C₁-C₃₀alkoxy, C₁₋₄haloalkyl, optionally substituted C₂₋₄alkenyl, optionally substituted C₂₋₄alkynyl, optionally substituted C₁₋₃₀alkyl-CO₂H, or a nitrogen-protecting group; X^{L2} is O or S; Y^{L3} is O, S, NH, or a bond; R^{L3B} is H or optionally substituted alkyl; * is bond to R^{H}; and R^{H} is 4-8 membered heterocyclyl comprising 1, 2 or 3 heteroatoms selected independently from N, O and S, and the heterocyclyl is optionally substituted with 1, 2, 3 or 4 independently selected substituents, and, optionally, the heterocyclyl comprises at least one nitrogen atom, or R^{H} is

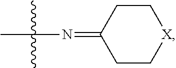

where X is O, NR^{L}, S, or CH₂; R^{L} is hydrogen, a ligand, a linker covalently bonded to one or more ligands, aliphatic and aromatic alkyl, alkylester, alkylamine, dimethylamino alkyl, alkylether, alkylthioether, heteroaromatic alkyl, allyl, vinyl, alkyl groups functionalized with disulfide, oxime, ketone, acetal, hemiacetal, cleavable peptides, or cleavable sugars; and R¹³ and R¹⁴ are independently -L²-R^{H2}, where L² is a linker; and R^{H2} is 4-8 membered heterocyclyl comprising 1, 2 or 3 heteroatoms selected independently from N, O and S, and the heterocyclyl is optionally substituted with 1, 2, 3 or 4 independently selected substituents, and, optionally at least one of R¹³ and R¹⁴ is -L²-R^{H2}.

In some embodiments of any one of the aspects described herein, R⁵ is -L¹-R^{H}.

In some embodiments of any one of the aspects, R⁵ is —O—N(R¹³)R¹⁴. It is noted, when R⁵ is —O—N(R¹³)R¹⁴, R¹³ and R¹⁴ can be same or different. Accordingly, in some embodiments of any one of the aspects described herein, R¹³ and R¹⁴ are same. In some embodiments of anyone of the aspects described herein, R¹³ and R¹⁴ are different.

In embodiments of the various aspects described herein, one or both of R¹³ and R¹⁴ can be -L²-R^{H2}.

In some embodiments of any one of the aspects described herein, at least one (e.g., one or both) of R¹³ and R¹⁴ is —(CH₂)ₘ—R^{H2} or

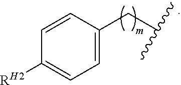

In some embodiments of any one of the aspects described herein R⁵ is N₃.

In some embodiments of any one of the aspects described herein, R⁵ is

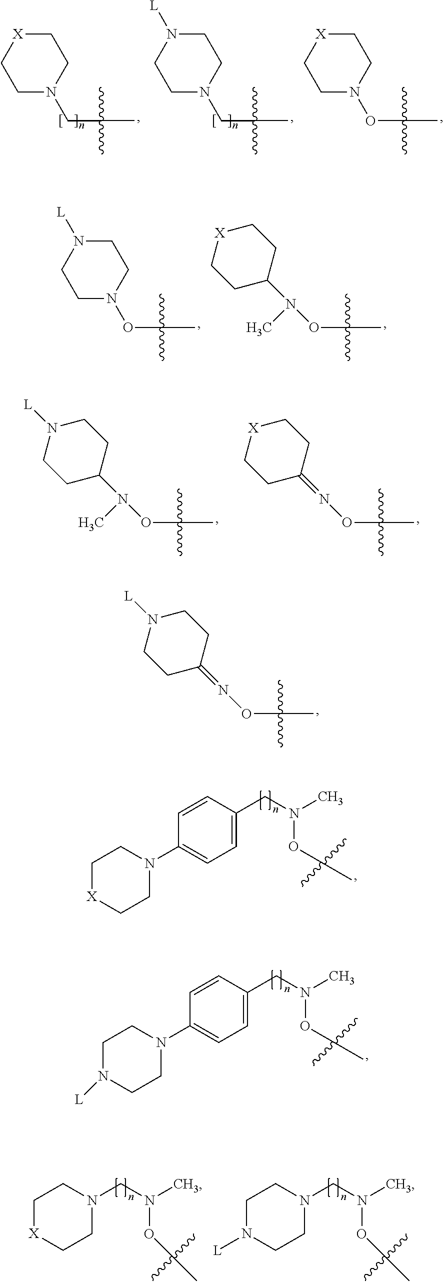

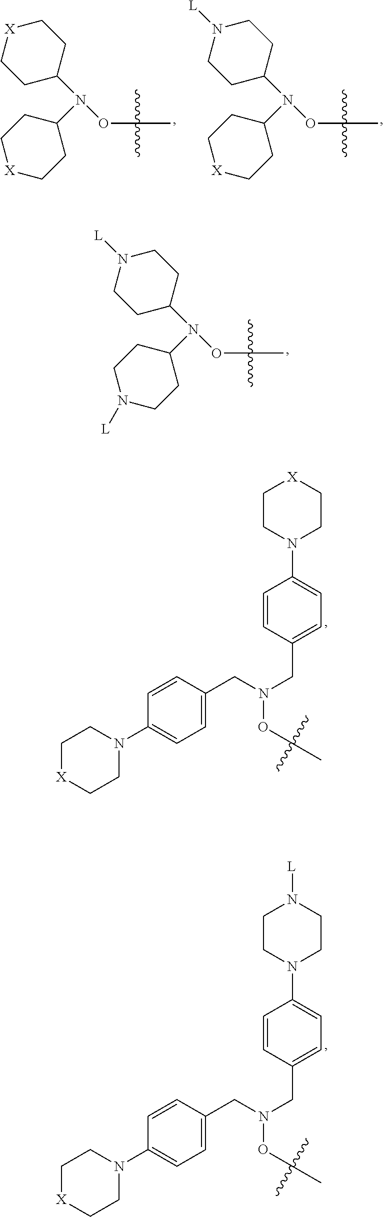

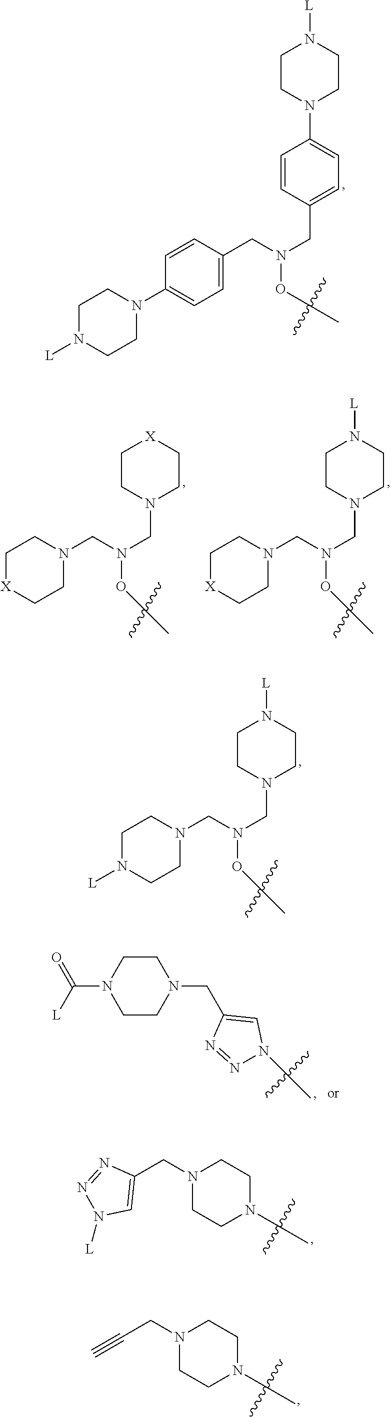

where n is 0 an integer selected from 1 to 30 (e.g., from 1 to 20, such as 1, 2, 3, 4, 5, or 6); X is ONH, S or CH₂; and L is a ligand or a linker covalently linked to one or more ligands (e.g., L is aliphatic and aromatic alkyl, alkylester, alkylamine, dimethylamino alkyl, alkylether, alkylthioether, heteroaromatic alkyl, allyl, vinyl, alkyl groups functionalized with disulfide, oxime, ketone, acetal, hemiacetal, cleavable peptides, or cleavable sugars.

In some embodiments of any one of the aspects described herein, R⁵ is

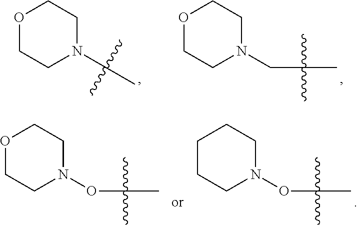 [SMILES: *N1CCOCC1 ; *CN1CCOCC1 ; *ON1CCOCC1 ; *ON1CCCCC1]

### R³

In some embodiments of any one of the aspects described herein, R³ is a reactive phosphorus group, hydrogen, halogen, —OR²³², —SR²³³, optionally substituted C₁₋₃₀alkyl, C₁₋₃₀haloalkyl, optionally substituted C₂₋₃₀alkenyl, optionally substituted C₂₋₃₀alkynyl, or optionally substituted C₁₋₃₀alkoxy, amino (NH₂), alkylamino, dialkylamino, heterocyclyl, arylamino, diarylamino, heteroarylamino, diheteroarylamino, amino acid, —O(CH₂CH₂O)ᵣCH₂CH₂OR²³⁴, cyano, alkyl-thio-alkyl, thioalkoxy, cycloalkyl, aryl, heteroaryl, —NH(CH₂CH₂NH)ₛCH₂CH₂—R²³⁵, NHC(O)R²³⁶, a lipid, a linker covalently attached to a lipid, a ligand or a linker covalently attached to a ligand.

R²³² can be H, hydroxyl protecting group, optionally substituted C₁₋₃₀alkyl, C₁₋₃₀haloalkyl, optionally substituted C₂₋₃₀alkenyl, optionally substituted C₂₋₃₀alkynyl, or optionally substituted C₁₋₃₀alkoxy, cycloalkyl, heterocyclyl, aryl, heteroaryl. R²³³ can be H, sulfur protecting group, optionally substituted C₁₋₃₀alkyl, C₁₋₃₀haloalkyl, optionally substituted C₂₋₃₀alkenyl, optionally substituted C₂₋₃₀alkynyl, or optionally substituted C₁₋₃₀alkoxy, cycloalkyl, heterocyclyl, aryl, heteroaryl. R²³⁴ can be H, hydroxyl protecting group, optionally substituted C₁₋₃₀alkyl, C₁₋₃₀haloalkyl, optionally substituted C₂₋₃₀alkenyl, optionally substituted C₂₋₃₀alkynyl, or optionally substituted C₁₋₃₀alkoxy, cycloalkyl, heterocyclyl, aryl, heteroaryl. R²³⁵ can be hydrogen, halogen, hydroxyl, protected hydroxyl, optionally substituted C₁₋₃₀alkyl, C₁₋₃₀haloalkyl, optionally substituted C₂₋₃₀alkenyl, optionally substituted C₂₋₃₀alkynyl, or optionally substituted C₁₋₃₀alkoxy, amino (NH₂), alkylamino, dialkylamino, heterocyclyl, arylamino, diarylamino, heteroarylamino, diheteroarylamino, amino acid, cyano, alkyl-thio-alkyl, thioalkoxy, cycloalkyl, aryl, or heteroaryl. R²³⁶ can be hydrogen, halogen, hydroxyl, protected hydroxyl, optionally substituted C₁₋₃₀alkyl, C₁₋₃₀haloalkyl, optionally substituted C₂₋₃₀alkenyl, optionally substituted C₂₋₃₀alkynyl, or optionally substituted C₁₋₃₀alkoxy, amino (NH₂), alkylamino, dialkylamino, heterocyclyl, arylamino, diarylamino, heteroarylamino, diheteroarylamino, amino acid, cyano, alkyl-thio-alkyl, thioalkoxy, cycloalkyl, aryl, or heteroaryl.

In some embodiments of any one of the aspects described herein, R³ is a reactive phosphorus group.

Without wishing to be bound by a theory, reactive phosphorus groups are useful for forming internucleoside linkages including for example phosphodiester and phosphorothioate internucleoside linkages. Such reactive phosphorus groups are known in the art and contain phosphorus atoms in P^{III} or P^{V} valence state including, but not limited to, phosphoramidite, H-phosphonate, phosphate triesters and phosphorus containing chiral auxiliaries. Reactive phosphorous group in the form of phosphoramidites (P^{III} chemistry) as reactive phosphites are a preferred reactive phosphorous group for solid phase oligonucleotide synthesis. The intermediate phosphite compounds are subsequently oxidized to the Pv state using known methods to yield phosphodiester or phosphorothioate internucleoside linkages.

In some embodiments of any one of the aspects described herein, the reactive phosphorous group is —OP(OR^{P})(N(R^{P2})₂), —OP(SR^{P})(N(R^{P2})₂), —OP(O)(OR^{P})(N(R^{P2})₂), —OP(S)(OR^{P})(N(R^{P2})₂), —OP(O)(SR^{P})(N(R^{P2})₂), —OP(O)(OR^{P})H, —OP(S)(OR^{P})H, —OP(O)(SR^{P})H, —OP(O)(OR^{P})R^{P3}, —OP(S)(OR^{P})R^{P3}, or —OP(O)(SR^{P})R^{P3}. For example, the reactive phosphorous group is —OP(OR^{P})(N(R^{P2})₂).

In some embodiments of any one of the aspects, R^{P} is an optionally substituted C₁₋₆alkyl. For example, R^{P} is a C₁₋₆alkyl, optionally substituted with 1, 2, 3, 4 or 5 substituents independently selected from OH, CN, SC(O)Ph, oxo (═O), SH, SO₂NH₂, SO₂(C₁-C₄)alkyl, SO₂NH(C₁-C₄)alkyl, halogen, carbonyl, thiol, cyano, NH₂, NH(C₁-C₄)alkyl, N[(C₁-C₄)alkyl]₂, C(O)NH₂, COOH, COOMe, acetyl, (C₁-C₈)alkyl, O(C₁-C₈)alkyl (i.e., C₁-C₈alkoxy), O(C₁-C₈)haloalkyl, (C₂-C₈)alkenyl, (C₂-C₈)alkynyl, haloalkyl, thioalkyl, cyanomethylene, alkylaminyl, aryl, heteroaryl, substituted aryl, NH₂—C(O)-alkylene, NH(Me)-C(O)-alkylene, CH₂—C(O)— alkyl, C(O)— alkyl, alkylcarbonylaminyl, CH₂—[CH(OH)]ₘ—(CH₂)ₚ—OH, CH₂—[CH(OH)]ₘ—(CH₂)ₚ—NH₂ or CH₂-aryl-alkoxy, where “m” and “p” are independently 1, 2, 3, 4, 5 or 6. In some embodiments, R^{P} is a C₁₋₆alkyl, optionally substituted with a CN or —SC(O)Ph. For example, R^{P} is cyanoethyl (—CH₂CH₂CN).

In the reactive phosphorous groups, each R^{P2} is independently optionally substituted C₁₋₆alkyl. For example, each R^{P2} can be independently selected from methyl, ethyl, propyl, isopropyl, n-butyl, iso-butyl, pentyl or hexyl. It is noted that when two or more R^{P2} groups are present in the reactive phosphorous group, they can be same or different. Thus, in some none-limiting examples, when two or more R^{P2} groups are present, the R^{P2} groups are different. In some other non-limiting examples, when two or more R^{P2} groups are present, the R^{P2} groups are same. In some embodiments of any one of the aspects, each R^{P2} is isopropyl.

In some embodiments of any one of the aspects, both R^{P2} taken together with the nitrogen atom to which they are attached form an optionally substituted 3-8 membered heterocyclyl. Exemplary heterocyclyls include, but are not limited to, pyrrolidinyl, piperazinyl, dioxanyl, morpholinyl, tetrahydrofuranyl, piperidyl, 4-morpholyl, 4-piperazinyl, pyrrolidinyl, perhydropyrrolizinyl, 1,4-diazaperhydroepinyl, 1,3-dioxanyl, 1,4-dioxanyland the like, each of which can be optionally substituted with 1, 2 or 3 substituents independently selected from OH, CN, SC(O)Ph, oxo (═O), SH, SO₂NH₂, SO₂(C₁-C₄)alkyl, SO₂NH(C₁-C₄)alkyl, halogen, carbonyl, thiol, cyano, NH₂, NH(C₁-C₄)alkyl, N[(C₁-C₄)alkyl]₂, C(O)NH₂, COOH, COOMe, acetyl, (C₁-C₈)alkyl, O(C₁-C₈)alkyl (i.e., C₁-C₈alkoxy), O(C₁-C₈)haloalkyl, (C₂-C₈)alkenyl, (C₂-C₈)alkynyl, haloalkyl, thioalkyl, cyanomethylene, alkylaminyl, aryl, heteroaryl, substituted aryl, NH₂—C(O)— alkylene, NH(Me)—C(O)-alkylene, CH₂—C(O)— alkyl, C(O)— alkyl, alkylcarbonylaminyl, CH₂—[CH(OH)]ₘ—(CH₂)ₚ—OH, CH₂—[CH(OH)]ₘ—(CH₂)ₚ—NH₂ or CH₂-aryl-alkoxy, where “m” and “p” are independently 1, 2, 3, 4, 5 or 6.

In some embodiments of any one of the aspects, R^{P} and one of R^{P2} taken together with the atoms to which they are attached form an optionally substituted 4-8 membered heterocyclyl. Exemplary heterocyclyls include, but are not limited to, pyrrolidinyl, piperazinyl, dioxanyl, morpholinyl, tetrahydrofuranyl, piperidyl, 4-morpholyl, 4-piperazinyl, pyrrolidinyl, perhydropyrrolizinyl, 1,4-diazaperhydroepinyl, 1,3-dioxanyl, 1,4-dioxanyland the like, each of which can be optionally substituted with 1, 2 or 3 substituents independently selected from OH, CN, SC(O)Ph, oxo (═O), SH, SO₂NH₂, SO₂(C₁-C₄)alkyl, SO₂NH(C₁-C₄)alkyl, halogen, carbonyl, thiol, cyano, NH₂, NH(C₁-C₄)alkyl, N[(C₁-C₄)alkyl]₂, C(O)NH₂, COOH, COOMe, acetyl, (C₁-C₈)alkyl, O(C₁-C₈)alkyl (i.e., C₁-C₈alkoxy), O(C₁-C₈)haloalkyl, (C₂-C₈)alkenyl, (C₂-C₈)alkynyl, haloalkyl, thioalkyl, cyanomethylene, alkylaminyl, aryl, heteroaryl, substituted aryl, NH₂—C(O)— alkylene, NH(Me)—C(O)-alkylene, CH₂—C(O)— alkyl, C(O)— alkyl, alkylcarbonylaminyl, CH₂—[CH(OH)]ₘ—(CH₂)ₚ—OH, CH₂—[CH(OH)]ₘ—(CH₂)ₚ—NH₂ or CH₂-aryl-alkoxy, where “m” and “p” are independently 1, 2, 3, 4, 5 or 6.

In the reactive phosphorous groups, each R^{P3} is independently optionally substituted C₁₋₆alkyl. For example, R^{P3} can be a C₁₋₆alkyl, optionally substituted with 1, 2, 3, 4 or 5 substituents independently selected from OH, CN, SC(O)Ph, oxo (═O), SH, SO₂NH₂, SO₂(C₁-C₄)alkyl, SO₂NH(C₁-C₄)alkyl, halogen, carbonyl, thiol, cyano, NH₂, NH(C₁-C₄)alkyl, N[(C₁-C₄)alkyl]₂, C(O)NH₂, COOH, COOMe, acetyl, (C₁-C₈)alkyl, O(C₁-C₅)alkyl (i.e., C₁-C₈alkoxy), O(C₁-C₅)haloalkyl, (C₂-C₈)alkenyl, (C₂-C₅)alkynyl, haloalkyl, thioalkyl, cyanomethylene, alkylaminyl, aryl, heteroaryl, substituted aryl, NH₂—C(O)-alkylene, NH(Me)-C(O)-alkylene, CH₂C(O)— alkyl, C(O)— alkyl, alkylcarbonylaminyl, CH₂—[CH(OH)]ₘ—(CH₂)ₚ—OH, CH₂—[CH(OH)]ₘ—(CH₂)ₚ—NH₂ or CH₂-aryl-alkoxy, where “m” and “p” are independently 1, 2, 3, 4, 5 or 6. For example, R^{P3} is methyl, ethyl, propyl, isopropyl, n-butyl, iso-butyl, pentyl or hexyl, each of which can be optionally substituted with a NH₂, OH, C(O)NH₂, COOH, halo, SH, or C₁-C₆alkoxy.

In some embodiments of any one of the aspects, the reactive phosphorous group is —OP(OR^{P})(N(R^{P2})₂). For example, the reactive phosphorous group is —OP(OR^{P})(N(R^{P2})₂), where R^{P} is cyanoethyl (—CH₂CH₂CN) and each R^{P2} is isopropyl.

In some embodiments of any one of the aspects described herein, R³ is —OP(OR^{P})(N(R^{P2})₂), —OP(SR^{P})(N(R^{P2})₂), —OP(O)(OR^{P})(N(R^{P2})₂), —OP(S)(OR^{P})(N(R^{P2})₂), —OP(O)(SR^{P})(N(R^{P2})₂), —OP(O)(OR^{P})H, —OP(S)(OR^{P})H, —OP(O)(SR^{P})H, —OP(O)(OR^{P})R^{P3}, —OP(S)(OR^{P})R^{P3}, or —OP(O)(SR^{P})R^{P3}, where each R^{P} is cyanoethyl (—CH₂CH₂CN), each R^{P2} is independently optionally substituted C₁₋₆alkyl; and each R^{P3} is independently optionally substituted C₁₋₆alkyl.

In some embodiments of any one of the aspects, R³ is —OP(OR^{P})(N(R^{P2})₂), —OP(SR^{P})(N(R^{P2})₂), —OP(O)(OR^{P})(N(R^{P2})₂), —OP(S)(OR^{P})(N(R^{P2})₂), —OP(O)(SR^{P})(N(R^{P2})₂), —OP(O)(OR^{P})H, —OP(S)(OR^{P}) an optionally substituted C₁₋₆alkyl, where each R^{P} is cyanoethyl (—CH₂CH₂CN), each R^{P2} is independently optionally substituted C₁₋₆alkyl; and each R^{P3} is independently optionally substituted C₁₋₆alkyl.

In some embodiments of any one of the aspects, R³ is —OP(OR^{P})(N(R^{P2})₂). For example, the R³ is —OP(OR^{P})(N(R^{P2})₂), where R^{P} is cyanoethyl (—CH₂CH₂CN) and each R^{P2} is isopropyl.

In some embodiments of any one of the aspects, when R³ is —OR²³², R²³² can be hydrogen or a hydroxyl protecting group. For example, R²³² can be hydrogen in some embodiments of any one of the aspects described herein. In some embodiments, R²³ is —OC(O)CH₂CH₂CO₂H.

When R³ is —SR²³³, R²³³ can be hydrogen or a sulfur protecting group. Accordingly, in some embodiments of any one of the aspects, R²³³ is hydrogen.

When R³ is —O(CH₂CH₂O)ᵣCH₂CH₂OR²³⁴, r can be 1-50; R²³⁴ is independently for each occurrence H, C₁-C₃₀alkyl, cyclyl, heterocyclyl, aryl, heteroaryl, aralkyl, sugar or R²³⁵; and R²³⁵ is independently for each occurrence amino (NH₂), alkylamino, dialkylamino, arylamino, diarylamino, heteroarylamino, or diheteroaryl amino.

When R³ is —NH(CH₂CH₂NH)ₛCH₂CH₂—R²³⁵, s can be 1-50 and R²³⁵ can be independently for each occurrence amino (NH₂), alkylamino, dialkylamino, arylamino, diarylamino, heteroarylamino, or diheteroaryl amino.

In some embodiments of any one of the aspects described herein, R³ is hydrogen, halogen, —OR²³², or optionally substituted C₁-C₃₀alkoxy. For example, R³ is halogen, —OR²³², or optionally substituted C₁-C₃₀alkoxy. In some embodiments of any one of the aspects described herein, R³ is F, OH or optionally substituted C₁-C₃₀alkoxy.

In some embodiments of any one of the aspects described herein, R³ is C₁-C₃₀alkoxy optionally substituted with 1, 2, 3, 4 or 5 substituents independently selected from OH, CN, SC(O)Ph, oxo (═O), SH, SO₂NH₂, SO₂(C₁-C₄)alkyl, SO₂NH(C₁-C₄)alkyl, halogen, carbonyl, thiol, cyano, NH₂, NH(C₁-C₄)alkyl, N[(C₁-C₄)alkyl]₂, C(O)NH₂, COOH, COOMe, acetyl, (C₁-C₈)alkyl, O(C₁-C₈)alkyl (i.e., C₁-C₈alkoxy), O(C₁-C₈)haloalkyl, (C₂-C₈)alkenyl, (C₂-C₈)alkynyl, haloalkyl, thioalkyl, cyanomethylene, alkylaminyl, aryl, heteroaryl, substituted aryl, NH₂—C(O)-alkylene, NH(Me)—C(O)-alkylene, CH₂—C(O)— alkyl, C(O)— alkyl, alkylcarbonylaminyl, CH₂—[CH(OH)]ₘ—(CH₂)ₚ—OH, CH₂—[CH(OH)]ₘ—(CH₂)ₚ—NH₂ or CH₂-aryl-alkoxy, where “m” and “p” are independently 1, 2, 3, 4, 5 or 6. For example, R²³ is C₁-C₃₀alkoxy optionally substituted with a NH₂, OH, C(O)NH₂, COOH, halo, SH, or C₁-C₆alkoxy. In some embodiments of any one of the aspects described herein, R²³ is —O(CH₂)ₜCH₃, where t is 1-21. For example, tis 14, 15, 16, 17 or 18. In one non-limiting example, t is 16.

In some embodiments of any one of the aspects, R³ is —O(CH₂)ᵤR²³⁷, where u is 2-10; R²³⁷ is C₁-C₆alkoxy, amino (NH₂), CO₂H, OH or halo. For example, R²³⁷ is —CH₃ or NH₂. Accordingly, in some embodiments of any one of the aspects described herein, R³ is —O(CH₂)ᵤ—OMe or R²³ is —O(CH₂)ᵤNH₂.

In some embodiments of any one of the aspects described herein, u is 2, 3, 4, 5 or 6. For example, u is 2, 3 or 6. In one non-limiting example, u is 2. In another non-limiting example, u is 3 or 6.

In some embodiments of any one of the aspects described herein, R³ is a C₁-C₆haloalkyl. For example, R³ is a C₁-C₄haloalkyl. In some embodiments of any one of the aspects described herein, R²³ is —CF₃, —CF₂CF₃, —CF₂CF₂CF₃ or —CF₂(CF₃)₂.

In some embodiments of any one of the aspects described herein, R³ is —OCH(CH₂OR²³⁸)CH₂OR²³⁹, where R²³⁸ and R²³⁹ independently are H, optionally substituted C₁-C₃₀alkyl, optionally substituted C₂-C₃₀alkenyl or optionally substituted C₂-C₃₀alkynyl. For example, R²³⁸ and R²³⁹ independently are optionally substituted C₁-C₃₀alkyl.

In some embodiments of any one of the aspects described herein, R²³ is —CH₂C(O)NHR²³¹⁰, where R²³¹⁰ is H, optionally substituted C₁-C₃₀alkyl, optionally substituted C₂-C₃₀alkenyl or optionally substituted C₂-C₃₀alkynyl. For example, R²³¹⁰ is H or optionally substituted C₁-C₃₀alkyl. In some embodiments, R²³¹⁰ is optionally substituted C₁-C₆alkyl

### R²

In some embodiments of any one of the aspects described herein, R² is hydrogen, halogen, —OR²²², —SR²²³, optionally substituted C₁₋₃₀alkyl, C₁₋₃₀haloalkyl, optionally substituted C₂₋₃₀alkenyl, optionally substituted C₂₋₃₀alkynyl, or optionally substituted C₁₋₃₀alkoxy, amino (NH₂), alkylamino, dialkylamino, heterocyclyl, arylamino, diarylamino, heteroarylamino, diheteroarylamino, amino acid, —O(CH₂CH₂O)ᵣCH₂CH₂OR²²⁴, cyano, alkyl-thio-alkyl, thioalkoxy, cycloalkyl, aryl, heteroaryl, —NH(CH₂CH₂NH)ₛCH₂CH₂—R²²⁵, NHC(O)R²²⁶, a lipid, a linker covalently attached to a lipid, a ligand, a linker covalently attached to a ligand, or a reactive phosphorus group.

R²²² can be H, hydroxyl protecting group, optionally substituted C₁₋₃₀alkyl, C₁₋₃₀haloalkyl, optionally substituted C₂₋₃₀alkenyl, optionally substituted C₂₋₃₀alkynyl, or optionally substituted C₁₋₃₀alkoxy, cycloalkyl, heterocyclyl, aryl, heteroaryl. R²²³ can be H, sulfur protecting group, optionally substituted C₁₋₃₀alkyl, C₁₋₃₀haloalkyl, optionally substituted C₂₋₃₀alkenyl, optionally substituted C₂₋₃₀alkynyl, or optionally substituted C₁₋₃₀alkoxy, cycloalkyl, heterocyclyl, aryl, heteroaryl. R²²⁴ can be H, hydroxyl protecting group, optionally substituted C₁₋₃₀alkyl, C₁₋₃₀haloalkyl, optionally substituted C₂₋₃₀alkenyl, optionally substituted C₂₋₃₀alkynyl, or optionally substituted C₁₋₃₀alkoxy, cycloalkyl, heterocyclyl, aryl, heteroaryl. R²²⁵ can be hydrogen, halogen, hydroxyl, protected hydroxyl, optionally substituted C₁₋₃₀alkyl, C₁₋₃₀haloalkyl, optionally substituted C₂₋₃₀alkenyl, optionally substituted C₂₋₃₀alkynyl, or optionally substituted C₁₋₃₀alkoxy, amino (NH₂), alkylamino, dialkylamino, heterocyclyl, arylamino, diarylamino, heteroarylamino, diheteroarylamino, amino acid, cyano, alkyl-thio-alkyl, thioalkoxy, cycloalkyl, aryl, or heteroaryl. R²²⁶ can be hydrogen, halogen, hydroxyl, protected hydroxyl, optionally substituted C₁₋₃₀alkyl, C₁₋₃₀haloalkyl, optionally substituted C₂₋₃₀alkenyl, optionally substituted C₂₋₃₀alkynyl, or optionally substituted C₁₋₃₀alkoxy, amino (NH₂), alkylamino, dialkylamino, heterocyclyl, arylamino, diarylamino, heteroarylamino, diheteroarylamino, amino acid, cyano, alkyl-thio-alkyl, thioalkoxy, cycloalkyl, aryl, or heteroaryl.

In some embodiments of any one of the aspects described herein, R² is hydrogen, halogen, —OR²²², —SR²²³, optionally substituted C₁₋₃₀alkyl, C₁₋₃₀haloalkyl, optionally substituted C₂₋₃₀alkenyl, optionally substituted C₂₋₃₀alkynyl, or optionally substituted C₁₋₃₀alkoxy, amino (NH₂), alkylamino, dialkylamino, heterocyclyl, arylamino, diarylamino, heteroarylamino, diheteroarylamino, amino acid, —O(CH₂CH₂O)ᵣCH₂CH₂OR²²⁴, cyano, alkyl-thio-alkyl, thioalkoxy, cycloalkyl, aryl, heteroaryl, —NH(CH₂CH₂NH)ₛCH₂CH₂—R²²⁵, NHC(O)R²²⁴.

In some embodiments of any one of the aspects described herein, R² is hydrogen, hydroxyl, protected hydroxyl, halogen, optionally substituted C₁₋₃₀ alkyl, optionally substituted C₂₋₃₀alkenyl, optionally substituted C₂₋₃₀alkynyl, optionally substituted C₁₋₃₀ alkoxy, alkoxyalkyl (e.g., methoxyethyl), alkoxyalkylamine, alkoxyoxycarboxylate, amino, alkylamino, dialkylamino, —O—C₄₋₃₀alkyl-ON(CH₂R⁸)(CH₂R⁹), or —O—C₄₋₃₀alkyl-ON(CH₂R⁸)(CH₂R⁹). For example, R² is hydrogen, hydroxyl, protected hydroxyl, halogen, optionally substituted C₁₋₃₀ alkoxy, alkoxyalkyl (e.g., methoxyethyl), alkoxyalkylamine, alkoxyoxycarboxylate, amino, alkylamino, or dialkylamino.

In some embodiments of any one of the aspects, R² is hydrogen, hydroxyl, protected hydroxyl, halogen, optionally substituted C₁₋₃₀ alkoxy, or alkoxyalkyl (e.g., methoxyethyl. In some embodiments of any one of the aspects, R² is hydrogen, hydroxyl, protected hydroxyl, fluoro or methoxy.

In some embodiments of any one of the aspects R² is halogen. For example, R² can be fluoro, chloro, bromo or iodo. In some embodiments of any one of the aspects described herein, R² is fluoro.

In some embodiments of any one of the aspects described herein, R² is hydrogen, fluoro or methoxy.

In some embodiments of any one of the aspects, when R² is OR²²², R²²² can be hydrogen or a hydroxyl protecting group.

When R² is —SR²²³, R²²³ can be hydrogen or a sulfur protecting group. Accordingly, in some embodiments of any one of the aspects, R²²³ is hydrogen.

When R² is —O(CH₂CH₂O)ᵣCH₂CH₂OR²²⁴, r can be 1-50; R²²⁴ is independently for each occurrence H, C₁-C₃₀alkyl, cyclyl, heterocyclyl, aryl, heteroaryl, aralkyl, sugar or R²²⁵; and R²²⁵ is independently for each occurrence amino (NH₂), alkylamino, dialkylamino, arylamino, diarylamino, heteroarylamino, or diheteroaryl amino.

When R² is —NH(CH₂CH₂NH)ₛCH₂CH₂—R²²⁵, s can be 1-50 and R²²⁵ can be independently for each occurrence amino (NH₂), alkylamino, dialkylamino, arylamino, diarylamino, heteroarylamino, or diheteroaryl amino.

In some embodiments of any one of the aspects described herein, R² is hydrogen, halogen, —OR²²², or optionally substituted C₁-C₃₀alkoxy. For example, R² is halogen, —OR²²², or optionally substituted C₁-C₃₀alkoxy. In some embodiments of any one of the aspects described herein, R² is F, OH or optionally substituted C₁-C₃₀alkoxy.

In some embodiments of any one of the aspects described herein, R² is C₁-C₃₀alkoxy optionally substituted with 1, 2, 3, 4 or 5 substituents independently selected from OH, CN, SC(O)Ph, oxo (═O), SH, SO₂NH₂, SO₂(C₁-C₄)alkyl, SO₂NH(C₁-C₄)alkyl, halogen, carbonyl, thiol, cyano, NH₂, NH(C₁-C₄)alkyl, N[(C₁-C₄)alkyl]₂, C(O)NH₂, COOH, COOMe, acetyl, (C₁-C₈)alkyl, O(C₁-C₅)alkyl (i.e., C₁-C₈alkoxy), O(C₁-C₅)haloalkyl, (C₂-C₈)alkenyl, (C₂-C₈)alkynyl, haloalkyl, thioalkyl, cyanomethylene, alkylaminyl, aryl, heteroaryl, substituted aryl, NH₂—C(O)-alkylene, NH(Me)—C(O)-alkylene, CH₂—C(O)— alkyl, C(O)— alkyl, alkylcarbonylaminyl, CH₂—[CH(OH)]ₘ—(CH₂)ₚ—OH, CH₂—[CH(OH)]ₘ—(CH₂)ₚ—NH₂ or CH₂-aryl-alkoxy, where “m” and “p” are independently 1, 2, 3, 4, 5 or 6. For example, R²² is C₁-C₃₀alkoxy optionally substituted with a NH₂, OH, C(O)NH₂, COOH, halo, SH, or C₁-C₆alkoxy. In some embodiments of any one of the aspects described herein, R² is —O(CH₂)ₜCH₃, where t is 1-21. For example, t is 14, 15, 16, 17 or 18. In one non-limiting example, t is 16.

In some embodiments of any one of the aspects, R² is —O(CH₂)ᵤR²²⁷, where u is 2-10; R²²⁷ is C₁-C₆alkoxy, amino (NH₂), CO₂H, OH or halo. For example, R²²⁷ is —CH₃ or NH₂. Accordingly, in some embodiments of any one of the aspects described herein, R² is —O(CH₂)ᵤ—OMe or R² is —O(CH₂)ᵤNH₂. In some embodiments of any one of the aspects described herein, u is 2, 3, 4, 5 or 6. For example, u is 2, 3 or 6. In one non-limiting example, u is 2. In another non-limiting example, u is 3 or 6.

In some embodiments of any one of the aspects described herein, R² is a C₁-C₆haloalkyl. For example, R² is a C₁-C₄haloalkyl. In some embodiments of any one of the aspects described herein, R² is —CF₃, —CF₂CF₃, —CF₂CF₂CF₃ or —CF₂(CF₃)₂.

In some embodiments of any one of the aspects described herein, R² is —OCH(CH₂OR²²⁸)CH₂OR²²⁹, where R²²⁸ and R²²⁹ independently are H, optionally substituted C₁-C₃₀alkyl, optionally substituted C₂-C₃₀alkenyl or optionally substituted C₂-C₃₀alkynyl. For example, R²²⁸ and R²²⁹ independently are optionally substituted C₁-C₃₀alkyl.

In some embodiments of any one of the aspects described herein, R² is —CH₂C(O)NHR²²¹⁰, where R²²¹⁰ is H, optionally substituted C₁-C₃₀alkyl, optionally substituted C₂-C₃₀alkenyl or optionally substituted C₂-C₃₀alkynyl. For example, R²²¹⁰ is H or optionally substituted C₁-C₃₀alkyl. In some embodiments, R²²¹⁰ is optionally substituted C₁-C₆alkyl.

In some embodiments of any one of the aspects, when R² is —OR²²², R²²² can be hydrogen or a hydroxyl protecting group.

When R² is —SR²²³, R²²³ can be hydrogen or a sulfur protecting group. Accordingly, in some embodiments of any one of the aspects, R²²³ is hydrogen.

When R² is —O(CH₂CH₂O)ᵣCH₂CH₂OR²²⁴, r can be 1-50; R²²⁴ is independently for each occurrence H, C₁-C₃₀alkyl, cyclyl, heterocyclyl, aryl, heteroaryl, aralkyl, sugar or R²²⁵; and R²²⁵ is independently for each occurrence amino (NH₂), alkylamino, dialkylamino, arylamino, diarylamino, heteroarylamino, or diheteroaryl amino.

When R² is —NH(CH₂CH₂NH)ₛCH₂CH₂—R²²⁵, s can be 1-50 and R²²⁵ can be independently for each occurrence amino (NH₂), alkylamino, dialkylamino, arylamino, diarylamino, heteroarylamino, or diheteroaryl amino.

In some embodiments of any one of the aspects described herein, R² is hydrogen, halogen, —OR²²², or optionally substituted C₁-C₃₀alkoxy. For example, R² is halogen, —OR²²², or optionally substituted C₁-C₃₀alkoxy. In some embodiments of any one of the aspects described herein, R² is F, OH or optionally substituted C₁-C₃₀alkoxy.

In some embodiments of any one of the aspects described herein, R² is C₁-C₃₀alkoxy optionally substituted with 1, 2, 3, 4 or 5 substituents independently selected from OH, CN, SC(O)Ph, oxo (═O), SH, SO₂NH₂, SO₂(C₁-C₄)alkyl, SO₂NH(C₁-C₄)alkyl, halogen, carbonyl, thiol, cyano, NH₂, NH(C₁-C₄)alkyl, N[(C₁-C₄)alkyl]₂, C(O)NH₂, COOH, COOMe, acetyl, (C₁-C₈)alkyl, O(C₁-C₅)alkyl (i.e., C₁-C₈alkoxy), O(C₁-C₈)haloalkyl, (C₂-C₈)alkenyl, (C₂-C₈)alkynyl, haloalkyl, thioalkyl, cyanomethylene, alkylaminyl, aryl, heteroaryl, substituted aryl, NH₂—C(O)-alkylene, NH(Me)-C(O)-alkylene, CH₂—C(O)-alkyl, C(O)-alkyl, alkylcarbonylaminyl, CH₂—[CH(OH)]ₘ—(CH₂)ₚ—OH, CH₂—[CH(OH)]ₘ—(CH₂)ₚ—NH₂ or CH₂-aryl-alkoxy, where “m” and “p” are independently 1, 2, 3, 4, 5 or 6. For example, R²² is C₁-C₃₀alkoxy optionally substituted with a NH₂, OH, C(O)NH₂, COOH, halo, SH, or C₁-C₆alkoxy. In some embodiments of any one of the aspects described herein, R² is —O(CH₂)ₜCH₃, where t is 1-21. For example, t is 14, 15, 16, 17 or 18. In one non-limiting example, t is 16.

In some embodiments of any one of the aspects, R² is —O(CH₂)ᵤR²²⁷, where u is 2-10; R²²⁷ is C₁-C₆alkoxy, amino (NH₂), CO₂H, OH or halo. For example, R²²⁷ is —CH₃ or NH₂. Accordingly, in some embodiments of any one of the aspects described herein, R² is —O(CH₂)ᵤ—OMe or R² is —O(CH₂)ᵤNH₂. In some embodiments of any one of the aspects described herein, u is 2, 3, 4, 5 or 6. For example, u is 2, 3 or 6. In one non-limiting example, u is 2. In another non-limiting example, u is 3 or 6.

In some embodiments of any one of the aspects described herein, R² is a C₁-C₆haloalkyl. For example, R² is a C₁-C₄haloalkyl. In some embodiments of any one of the aspects described herein, R² is-CF₃, —CF₂CF₃, —CF₂CF₂CF₃ or —CF₂ (CF₃)₂.

In some embodiments of any one of the aspects described herein, R² is —OCH(CH₂OR²²⁸)CH₂OR²²⁹, where R²²⁸ and R²²⁹ independently are H, optionally substituted C₁-C₃₀alkyl, optionally substituted C₂-C₃₀alkenyl or optionally substituted C₂-C₈alkynyl. For example, R²²⁸ and R²²⁹ independently are optionally substituted C₁-C₃₀alkyl.

In some embodiments of any one of the aspects described herein, R² is —CH₂C(O)NHR²²¹⁰, where R²²¹⁰ is H, optionally substituted C₁-C₃₀alkyl, optionally substituted C₂-C₃₀alkenyl or optionally substituted C₂-C₃₀alkynyl. For example, R²²¹⁰ is H or optionally substituted C₁-C₃₀alkyl. In some embodiments, R²²¹⁰ is optionally substituted C₁-C₆alkyl.

In some embodiments of any one of the aspects described herein, R² is a reactive phosphorus group. For example, R² is —OP(OR^{P})(N(R^{P2})₂), —OP(SR^{P})(N(R^{P2})₂), —OP(O)(OR^{P})(N(R^{P2})₂), —OP(S)(OR^{P})(N(R^{P2})₂), —OP(O)(SR^{P})(NR^{P2})₂, —OP(O)(OR^{P})H, —OP(S)(OR^{P})H, —OP(O)(SR^{P})H, —OP(O)(OR^{P})R^{P3}, —OP(S)(OR^{P})R^{P3}, or —OP(O)(SR^{P})R^{P3}, where each R^{P} is cyanoethyl (—CH₂CH₂CN), each R^{P2} is independently optionally substituted C₁₋₆alkyl; and each R^{P3} is independently optionally substituted C₁₋₆alkyl.

In some embodiments of any one of the aspects, R² is —OP(OR^{P})(N(R^{P2})₂), —OP(SR^{P})(N(R^{P2})₂), —OP(O)(OR^{P})(N(R^{P2})₂), —OP(S)(OR^{P})(N(R^{P2})₂), —OP(O)(SR^{P})(N(R^{P2})₂), —OP(O)(OR^{P})H, —OP(S)(OR^{P}) an optionally substituted C₁₋₆alkyl, where each R^{P} is cyanoethyl (—CH₂CH₂CN), each R^{P2} is independently optionally substituted C₁₋₆alkyl; and each R^{P3} is independently optionally substituted C₁₋₆alkyl.

In some embodiments of any one of the aspects, R² is —OP(OR^{P})(N(R^{P2})₂). For example, the R² is —OP(OR^{P})(N(R^{P2})₂), where R^{P} is cyanoethyl (—CH₂CH₂CN) and each R^{P2} is isopropyl.

It is noted that only one of R² and R³ can be a reactive phosphorus group. Preferably, R³ is a phosphorous group.

In some embodiments of any one of the aspects described herein, R² and R⁴ taken together are 4′-C(R¹⁰R¹¹)ᵥ—Y-2′ or 4′-Y—C(R¹⁰R¹¹)ᵥ-2′; v is 1, 2 or 3; where Y is —O—, —CH₂—, —CH(Me)-, —C(CH₃)₂—, —S—, —N(R¹²)—, —C(O)—, —C(S)—, —S(O)—, —S(O)₂—, —OC(O)—, —C(O)O—, —N(R¹²)C(O)—, or —C(O)N(R¹²)—; R¹⁰ and R¹¹ independently are H, optionally substituted C₁-C₆alkyl, optionally substituted C₂-C₆alkenyl or optionally substituted C₂-C₆alkynyl; R¹² is hydrogen, optionally substituted C₁₋₃₀alkyl, optionally substituted C₁-C₃₀alkoxy, C₁₋₄haloalkyl, optionally substituted C₂₋₄alkenyl, optionally substituted C₂₋₄alkynyl, optionally substituted C₁₋₃₀alky-CO₂H, or a nitrogen-protecting group.

In some embodiments of any one of the aspects, v is 1. In some other embodiments of any one of the aspects, v is 2.

In some embodiments, Y is O. For example, R² and R⁴ taken together are 4′-C(R¹⁰R¹¹)ᵥ—O-2′.

It is noted that R¹⁰ and R¹¹ attached to the same carbon can be same or different. For example, one of R¹⁰ and R¹¹ can be H and the other of the R¹⁰ and R¹¹ can be an optionally substituted C₁-C₆alkyl. In one non-limiting example, one of R¹⁰ and R¹¹ can be H and the other can be C₁-C₆alkyl, optionally substituted with 1, 2, 3, 4 or 5 substituents independently selected from OH, CN, SC(O)Ph, oxo (═O), SH, SO₂NH₂, SO₂(C₁-C₄)alkyl, SO₂NH(C₁-C₄)alkyl, halogen, carbonyl, thiol, cyano, NH₂, NH(C₁-C₄)alkyl, N[(C₁-C₄)alkyl]₂, C(O)NH₂, COOH, COOMe, acetyl, (C₁-C₅)alkyl, O(C₁-C₅)alkyl (i.e., C₁-C₈alkoxy), O(C₁-C₅)haloalkyl, (C₂-C₈)alkenyl, (C₂-C₈)alkynyl, haloalkyl, thioalkyl, cyanomethylene, alkylaminyl, aryl, heteroaryl, substituted aryl, NH₂—C(O)-alkylene, NH(Me)-C(O)-alkylene, CH₂—C(O)-alkyl, C(O)-alkyl, alkylcarbonylaminyl, CH₂—[CH(OH)]ₘ—(CH₂)ₚ—OH, CH₂—[CH(OH)]ₘ—(CH₂)ₚ—NH₂ or CH₂-aryl-alkoxy, where “m” and “p” are independently 1, 2, 3, 4, 5 or 6. For example, R¹⁰ and R¹¹ independently are H or C₁-C₃₀alkyl optionally substituted with a NH₂, OH, C(O)NH₂, COOH, halo, SH, or C₁-C₆alkoxy. In some embodiments of any one of the aspects, one of R¹⁰ and R¹¹ is H and the other is C₁-C₆alkyl, optionally substituted with a C₁-C₆alkoxy. For example, one of R¹⁰ and R¹¹ is H and the other is —CH₃ or CH₂OCH₃.

In some embodiments of any one of the aspects, R¹⁰ and R¹¹ attached to the same C are the same. For example, R¹⁰ and R¹¹ attached to the same C are H.

In some embodiments of any one of the aspects, R² and R⁴ taken together are 4′-CH₂—O-2′, 4′—CH(CH₃)—O-2′, 4′—CH(CH₂OCH₃)—O-2′, or 4′-CH₂CH₂—O-2′. For example, R² and R⁴ taken together are 4′-CH₂CH₂—O-2′.

In some embodiments of any one of the aspects described herein, R² and R⁴ taken together are 4′-C(R¹⁰R¹¹)ᵥ—Y-2′ or 4′-Y—C(R¹⁰R¹¹)ᵥ-2′; and R³ is a reactive phosphorous group, hydroxyl or protected hydroxyl.

In some embodiments of any one of the aspects described herein, R² is hydrogen, fluoro or methoxy; R³ is a reactive phosphorous group, hydroxyl or protected hydroxyl; and R⁴ is H.

### R⁴

In some embodiments of any one of the aspects described herein, R⁴ can be hydrogen, optionally substituted C₁₋₆alkyl, optionally substituted C₂₋₆alkenyl, optionally substituted C₂₋₆alkynyl, or optionally substituted C₁₋₆alkoxy. For example, R⁴ can be hydrogen, optionally substituted C₁₋₆alkyl or optionally substituted C₁₋₆alkoxy.

In some embodiments of any one of the aspects described herein, R⁴ is H.

### R²³

In some embodiments of any one of the aspects described herein, R²³ is a bond to an internucleotide linkage to a subsequent nucleoside, hydroxyl, protected hydroxyl, optionally substituted C₁₋₃₀ alkoxy, halogen, alkoxyalkyl (e.g., methoxyethyl), amino, alkylamino, dialkylamino, a 3′-oligonuclotide capping group (e.g., an inverted nucleotide or an inverted abasic nucleotide), a ligand, or a linker covalently bonded to one or more ligands (e.g., N-acetylgalactosamine (GalNac)).

In some embodiments of any one of the aspects described herein, R²³ is a bond to an internucleotide linkage to a subsequent nucleotide. It is noted that only one of R²³ and R₂₂ can be a bond to an internucleotide linkage to a subsequent nucleotide. Preferably, R²³ is a bond to an internucleotide linkage to a subsequent nucleotide.

In some embodiments of any one of the aspects described herein, R²³ is a hydrogen, halogen, —OR²³², —SR²³³, optionally substituted C₁₋₃₀alkyl, C₁₋₃₀haloalkyl, optionally substituted C₂₋₃₀alkenyl, optionally substituted C₂₋₃₀alkynyl, or optionally substituted C₁₋₃₀alkoxy, amino (NH₂), alkylamino, dialkylamino, heterocyclyl, arylamino, diarylamino, heteroarylamino, diheteroarylamino, amino acid, —O(CH₂CH₂O)ᵣCH₂CH₂OR²³⁴, cyano, alkyl-thio-alkyl, thioalkoxy, cycloalkyl, aryl, heteroaryl, —NH(CH₂CH₂NH)ₛCH₂CH₂—R²³⁵, NHC(O)R²³⁶, a lipid, a linker covalently attached to a lipid, a ligand or a linker covalently attached to a ligand.

R²³² can be H, hydroxyl protecting group, optionally substituted C₁₋₃₀alkyl, C₁₋₃₀haloalkyl, optionally substituted C₂₋₃₀alkenyl, optionally substituted C₂₋₃₀alkynyl, or optionally substituted C₁₋₃₀alkoxy, cycloalkyl, heterocyclyl, aryl, heteroaryl. R²³³ can be H, sulfur protecting group, optionally substituted C₁₋₃₀alkyl, C₁₋₃₀haloalkyl, optionally substituted C₂₋₃₀alkenyl, optionally substituted C₂₋₃₀alkynyl, or optionally substituted C₁₋₃₀alkoxy, cycloalkyl, heterocyclyl, aryl, heteroaryl. R²³⁴ can be H, hydroxyl protecting group, optionally substituted C₁₋₃₀alkyl, C₁₋₃₀haloalkyl, optionally substituted C₂₋₃₀alkenyl, optionally substituted C₂₋₃₀alkynyl, or optionally substituted C₁₋₃₀alkoxy, cycloalkyl, heterocyclyl, aryl, heteroaryl. R²³⁵ can be hydrogen, halogen, hydroxyl, protected hydroxyl, optionally substituted C₁₋₃₀alkyl, C₁₋₃₀haloalkyl, optionally substituted C₂₋₃₀alkenyl, optionally substituted C₂₋₃₀alkynyl, or optionally substituted C₁₋₃₀alkoxy, amino (NH₂), alkylamino, dialkylamino, heterocyclyl, arylamino, diarylamino, heteroarylamino, diheteroarylamino, amino acid, cyano, alkyl-thio-alkyl, thioalkoxy, cycloalkyl, aryl, or heteroaryl. R²³⁶ can be hydrogen, halogen, hydroxyl, protected hydroxyl, optionally substituted C₁₋₃₀alkyl, C₁₋₃₀haloalkyl, optionally substituted C₂₋₃₀alkenyl, optionally substituted C₂₋₃₀alkynyl, or optionally substituted C₁₋₃₀alkoxy, amino (NH₂), alkylamino, dialkylamino, heterocyclyl, arylamino, diarylamino, heteroarylamino, diheteroarylamino, amino acid, cyano, alkyl-thio-alkyl, thioalkoxy, cycloalkyl, aryl, or heteroaryl.

In some embodiments of any one of the aspects, when R²³ is —OR²³², R²³² can be hydrogen or a hydroxyl protecting group. For example, R²³² can be hydrogen, a hydroxyl protecting group or an alkyl group (e.g., methoxy) in some embodiments of any one of the aspects described herein.

When R²³ is —SR²³³, R²³³ can be hydrogen or a sulfur protecting group. Accordingly, in some embodiments of any one of the aspects, R²³³ is hydrogen.

When R²³ is —O(CH₂CH₂O)ᵣCH₂CH₂OR²³⁴, r can be 1-50; R²³⁴ is independently for each occurrence H, C₁-C₃₀alkyl, cyclyl, heterocyclyl, aryl, heteroaryl, aralkyl, sugar or R²³⁵; and R²³⁵ is independently for each occurrence amino (NH₂), alkylamino, dialkylamino, arylamino, diarylamino, heteroarylamino, or diheteroaryl amino.

When R²³ is —NH(CH₂CH₂NH)ₛCH₂CH₂—R²³⁵, s can be 1-50 and R²³⁵ can be independently for each occurrence amino (NH₂), alkylamino, dialkylamino, arylamino, diarylamino, heteroarylamino, or diheteroaryl amino.

In some embodiments of any one of the aspects described herein, R²³ is hydrogen, halogen, —OR²³², or optionally substituted C₁-C₃₀alkoxy. For example, R²³ is halogen, OR²³², or optionally substituted C₁-C₃₀alkoxy. In some embodiments of any one of the aspects described herein, R²³ is F, OH or optionally substituted C₁-C₃₀alkoxy.

In some embodiments of any one of the aspects described herein, R²³ is C₁-C₃₀alkoxy optionally substituted with 1, 2, 3, 4 or 5 substituents independently selected from OH, CN, SC(O)Ph, oxo (═O), SH, SO₂NH₂, SO₂(C₁-C₄)alkyl, SO₂NH(C₁-C₄)alkyl, halogen, carbonyl, thiol, cyano, NH₂, NH(C₁-C₄)alkyl, N[(C₁-C₄)alkyl]₂, C(O)NH₂, COOH, COOMe, acetyl, (C₁-C₈)alkyl, O(C₁-C₅)alkyl (i.e., C₁-C₈alkoxy), O(C₁-C₅)haloalkyl, (C₂-C₈)alkenyl, (C₂-C₈)alkynyl, haloalkyl, thioalkyl, cyanomethylene, alkylaminyl, aryl, heteroaryl, substituted aryl, NH₂—C(O)-alkylene, NH(Me)-C(O)-alkylene, CH₂—C(O)-alkyl, C(O)-alkyl, alkylcarbonylaminyl, CH₂—[CH(OH)]ₘ—(CH₂)ₚ—OH, CH₂—[CH(OH)]ₘ—(CH₂)ₚ—NH₂ or CH₂-aryl-alkoxy, where “m” and “p” are independently 1, 2, 3, 4, 5 or 6. For example, R²³ is C₁-C₃₀alkoxy optionally substituted with a NH₂, OH, C(O)NH₂, COOH, halo, SH, or C₁-C₆alkoxy. In some embodiments of any one of the aspects described herein, R²³ is —O(CH₂)ₜCH₃, where t is 1-21. For example, tis 14, 15, 16, 17 or 18. In one non-limiting example, t is 16.

In some embodiments of any one of the aspects, R²³ is —O(CH₂)ᵤR²³⁷, where u is 2-10; R²³⁷ is C₁-C₆alkoxy, amino (NH₂), CO₂H, OH or halo. For example, R²³⁷ is —CH₃ or NH₂. Accordingly, in some embodiments of any one of the aspects described herein, R²³ is —O(CH₂)ᵤ—OMe or R²³ is —O(CH₂)ᵤNH₂. In some embodiments of any one of the aspects described herein, u is 2, 3, 4, 5 or 6. For example, u is 2, 3 or 6. In one non-limiting example, u is 2. In another non-limiting example, u is 3 or 6.

In some embodiments of any one of the aspects described herein, R²³ is a C₁-Chaloalkyl. For example, R²³ is a C₁-C₄haloalkyl. In some embodiments of any one of the aspects described herein, R²³ is-CF₃, —CF₂CF₃, —CF₂CF₂CF₃ or —CF₂ (CF₃)₂.

In some embodiments of any one of the aspects described herein, R²³ is —OCH(CH₂OR²³⁸)CH₂OR²³⁹, where R²³⁸ and R²³⁹ independently are H, optionally substituted C₁-C₃₀alkyl, optionally substituted C₂-C₃₀alkenyl or optionally substituted C₂-C₃₀alkynyl. For example, R²³⁸ and R²³⁹ independently are optionally substituted C₁-C₃₀alkyl.

In some embodiments of any one of the aspects described herein, R²³ is —CH₂C(O)NHR²³¹⁰, where R²³¹⁰ is H, optionally substituted C₁-C₃₀alkyl, optionally substituted C₂-C₃₀alkenyl or optionally substituted C₂-C₃₀alkynyl. For example, R²³¹⁰ is H or optionally substituted C₁-C₃₀alkyl. In some embodiments, R²³¹⁰ is optionally substituted C₁-C₆alkyl.

### R²²

In some embodiments of any one of the aspects described herein, R²² is hydrogen, halogen, —OR²²², —SR²²³, optionally substituted C₁₋₃₀alkyl, C₁₋₃₀haloalkyl, optionally substituted C₂-30alkenyl, optionally substituted C₂₋₃₀alkynyl, or optionally substituted C₁₋₃₀alkoxy, amino (NH₂), alkylamino, dialkylamino, heterocyclyl, arylamino, diarylamino, heteroarylamino, diheteroarylamino, amino acid, —O(CH₂CH₂O)ᵣCH₂CH₂OR²²⁴, cyano, alkyl-thio-alkyl, thioalkoxy, cycloalkyl, aryl, heteroaryl, —NH(CH₂CH₂NH)ₛCH₂CH₂—R²²⁵, NHC(O)R²²⁶, a lipid, a linker covalently attached to a lipid, a ligand, a linker covalently attached to a ligand, or a reactive phosphorus group.

R²²² can be H, hydroxyl protecting group, optionally substituted C₁₋₃₀alkyl, C₁₋₃₀haloalkyl, optionally substituted C₂₋₃₀alkenyl, optionally substituted C₂₋₃₀alkynyl, or optionally substituted C₁₋₃₀alkoxy, cycloalkyl, heterocyclyl, aryl, heteroaryl. R²²³ can be H, sulfur protecting group, optionally substituted C₁₋₃₀alkyl, C₁₋₃₀haloalkyl, optionally substituted C₂₋₃₀alkenyl, optionally substituted C₂₋₃₀alkynyl, or optionally substituted C₁₋₃₀alkoxy, cycloalkyl, heterocyclyl, aryl, heteroaryl. R²²⁴ can be H, hydroxyl protecting group, optionally substituted C₁₋₃₀alkyl, C₁₋₃₀haloalkyl, optionally substituted C₂₋₃₀alkenyl, optionally substituted C₂₋₃₀alkynyl, or optionally substituted C₁₋₃₀alkoxy, cycloalkyl, heterocyclyl, aryl, heteroaryl. R²²⁵ can be hydrogen, halogen, hydroxyl, protected hydroxyl, optionally substituted C₁₋₃₀alkyl, C₁₋₃₀haloalkyl, optionally substituted C₂₋₃₀alkenyl, optionally substituted C₂₋₃₀alkynyl, or optionally substituted C₁₋₃₀alkoxy, amino (NH₂), alkylamino, dialkylamino, heterocyclyl, arylamino, diarylamino, heteroarylamino, diheteroarylamino, amino acid, cyano, alkyl-thio-alkyl, thioalkoxy, cycloalkyl, aryl, or heteroaryl. R²²⁶ can be hydrogen, halogen, hydroxyl, protected hydroxyl, optionally substituted C₁₋₃₀alkyl, C₁₋₃₀haloalkyl, optionally substituted C₂₋₃₀alkenyl, optionally substituted C₂₋₃₀alkynyl, or optionally substituted C₁₋₃₀alkoxy, amino (NH₂), alkylamino, dialkylamino, heterocyclyl, arylamino, diarylamino, heteroarylamino, diheteroarylamino, amino acid, cyano, alkyl-thio-alkyl, thioalkoxy, cycloalkyl, aryl, or heteroaryl.

In some embodiments of any one of the aspects described herein, R²² is hydrogen, halogen, —OR²²², —SR²²³, optionally substituted C₁₋₃₀alkyl, C₁₋₃₀haloalkyl, optionally substituted C₂₋₃₀alkenyl, optionally substituted C₂₋₃₀alkynyl, or optionally substituted C₁₋₃₀alkoxy, amino (NH₂), alkylamino, dialkylamino, heterocyclyl, arylamino, diarylamino, heteroarylamino, diheteroarylamino, amino acid, —O(CH₂CH₂O)ᵣCH₂CH₂OR²²⁴, cyano, alkyl-thio-alkyl, thioalkoxy, cycloalkyl, aryl, heteroaryl, —NH(CH₂CH₂NH)ₛCH₂CH₂-R^{225x}, NHC(O)R²²⁴.

In some embodiments of any one of the aspects described herein, R²² is hydrogen, hydroxyl, protected hydroxyl, halogen, optionally substituted C₁₋₃₀ alkyl, optionally substituted C₂₋₃₀alkenyl, optionally substituted C₂₋₃₀alkynyl, optionally substituted C₁₋₃₀ alkoxy, alkoxyalkyl (e.g., methoxyethyl), alkoxyalkylamine, alkoxyoxycarboxylate, amino, alkylamino, dialkylamino, —O—C₄₋₃₀alkyl-ON(CH₂R⁸)(CH₂R⁹), or —O—C₄₋₃₀alkyl-ON(CH₂R⁸)(CH₂R^{9x}. For example, R²² is hydrogen, hydroxyl, protected hydroxyl, halogen, optionally substituted C₁₋₃₀ alkoxy, alkoxyalkyl (e.g., methoxyethyl), alkoxyalkylamine, alkoxyoxycarboxylate, amino, alkylamino, or dialkylamino.

In some embodiments of any one of the aspects, R²² is hydrogen, hydroxyl, protected hydroxyl, halogen, optionally substituted C₁₋₃₀ alkoxy, or alkoxyalkyl (e.g., methoxyethyl. In some embodiments of any one of the aspects, R²² is hydrogen, hydroxyl, protected hydroxyl, fluoro or methoxy.

In some embodiments of any one of the aspects R²² is halogen. For example, R²² can be fluoro, chloro, bromo or iodo. In some embodiments of any one of the aspects described herein, R²² is fluoro.

In some embodiments of any one of the aspects described herein, R²² is hydrogen, fluoro or methoxy.

In some embodiments of any one of the aspects described herein, R²² and R²⁴ taken together are 4′-C(R¹⁰R¹¹)ᵥ—Y-2′ or 4′-Y—C(R¹⁰R¹¹)ᵥ-2′; v is 1, 2 or 3; where Y is —O—, —CH₂—, —CH(Me)-, —C(CH₃)₂—, —S—, —N(R¹²)—, —C(O)—, —C(S)—, —S(O)—, —S(O)₂—, —OC(O)—, —C(O)O—, —N(R¹²)C(O)—, or —C(O)N(R¹²)—; R¹⁰ and R¹¹ independently are H, optionally substituted C₁-C₆alkyl, optionally substituted C₂-C₆alkenyl or optionally substituted C₂-C₆alkynyl; R¹² is hydrogen, optionally substituted C₁₋₃₀alkyl, optionally substituted C₁-C₃₀alkoxy, C₁₄haloalkyl, optionally substituted C₂₋₄alkenyl, optionally substituted C₂₋₄alkynyl, optionally substituted C₁₋₃₀alky-CO₂H, or a nitrogen-protecting group. In some embodiments of any one of the aspects, v is 1. In some other embodiments of any one of the aspects, v is 2. In some embodiments, Y is O. For example, R² and R⁴ taken together are 4′-C(R¹⁰R¹¹)ᵥ—O-2′.

It is noted that R¹⁰ and R¹¹ attached to the same carbon can be same or different. For example, one of R¹⁰ and R¹¹ can be H and the other of the R¹⁰ and R¹¹ can be an optionally substituted C₁-C₆alkyl. In one non-limiting example, one of R¹⁰ and R¹¹ can be H and the other can be C₁-C₆alkyl, optionally substituted with 1, 2, 3, 4 or 5 substituents independently selected from OH, CN, SC(O)Ph, oxo (═O), SH, SO₂NH₂, SO₂(C₁-C₄)alkyl, SO₂NH(C₁-C₄)alkyl, halogen, carbonyl, thiol, cyano, NH₂, NH(C₁-C₄)alkyl, N[(C₁-C₄)alkyl]₂, C(O)NH₂, COOH, COOMe, acetyl, (C₁-C₅)alkyl, O(C₁-C₅)alkyl (i.e., C₁-C₈alkoxy), O(C₁-C₅)haloalkyl, (C₂-C₈)alkenyl, (C₂-C₈)alkynyl, haloalkyl, thioalkyl, cyanomethylene, alkylaminyl, aryl, heteroaryl, substituted aryl, NH₂—C(O)-alkylene, NH(Me)-C(O)-alkylene, CH₂C(O)-alkyl, C(O)-alkyl, alkylcarbonylaminyl, CH₂—[CH(OH)]ₘ—(CH₂)ₚ—OH, CH₂—[CH(OH)]ₘ—(CH₂)ₚ—NH₂ or CH₂-aryl-alkoxy, where “m” and “p” are independently 1, 2, 3, 4, 5 or 6. For example, R¹⁰ and R¹¹ independently are H or C₁-C₃₀alkyl optionally substituted with a NH₂, OH, C(O)NH₂, COOH, halo, SH, or C₁-C₆alkoxy. In some embodiments of any one of the aspects, one of R¹⁰ and R¹¹ is H and the other is C₁-C₆alkyl, optionally substituted with a C₁-C₆alkoxy. For example, one of R¹⁰ and R¹¹ is H and the other is —CH₃ or CH₂OCH₃.

In some embodiments of any one of the aspects, R¹⁰ and R¹¹ attached to the same C are the same. For example, R¹⁰ and R¹¹ attached to the same C are H.

In some embodiments of any one of the aspects, R²² and R²⁴ taken together are 4′-CH₂-0-2′, 4′—CH(CH₃)—O-2′, 4′—CH(CH₂OCH₃)—O-2′, or 4′-CH₂CH₂—O-2′. For example, R²² and R²⁴ taken together are 4′-CH₂CH₂—O-2′.

In some embodiments of any one of the aspects described herein, R²² is a bond to an internucleotide linkage to a subsequent nucleoside.

R²⁴

In some embodiments of any one of the aspects described herein, R²⁴ can be hydrogen, optionally substituted C₁₋₆alkyl, optionally substituted C₂₋₆alkenyl, optionally substituted C₂₋₆alkynyl, or optionally substituted C₁₋₆alkoxy. For example, R²⁴ can be hydrogen, optionally substituted C₁₋₆alkyl or optionally substituted C₁₋₆alkoxy.

In some embodiments of any one of the aspects described herein, R²⁴ is H.

### L¹

In some embodiments of any one of the aspects described herein, L′ can be a linker.

For example, L′ can be a direct bond or an atom such as oxygen or sulfur, a unit such as NRLL, C(O), C(O) O, C(O)NR¹, SO, SO₂, SO₂NH or a chain of atoms, such as substituted or unsubstituted alkyl, substituted or unsubstituted alkenyl, or substituted or unsubstituted alkynyl, where one or more methylenes can be interrupted or terminated by O, S, S(O), SO₂, N(RLL) 2, C(O), cleavable linking group, substituted or unsubstituted aryl, substituted or unsubstituted heteroaryl, substituted or unsubstituted heterocyclic; where R^{LL} is hydrogen, acyl, aliphatic or substituted aliphatic

In some embodiments of any one of the aspects described herein, L′ is a bond or an optionally substituted alkylene. For example, L′ is a bond. In some other non-limiting examples, L′ is substituted or unsubstituted alkyl, substituted or unsubstituted alkenyl, or substituted or unsubstituted alkynyl, where one or more methylenes can be interrupted or terminated by O, S, S(O), SO₂, N(R^{LL})₂, C(O), cleavable linking group, substituted or unsubstituted aryl, substituted or unsubstituted heteroaryl, substituted or unsubstituted heterocyclic.

In some embodiments of any one of the aspects described herein, L′ is a bond, -L³-, C₁₋₃₀alkylene, C₂₋₃₀alkenylene, C₂₋₃₀alkynylene, *-L³-C₁₋₃₀alkylene *-L³-C₂₋₃₀alkenylene, or *-L³-C₂₋₃₀alkynylene.

In some embodiments of any one of the aspects described herein L′ is L³, where L³ is —O—, —N(R¹³)—, —S—, —C(O)—, —S(O)—, —S(O)₂—, —P(X^{L3})(Y^{L3}R^{L3}B)—, where R^{L3} is hydrogen, optionally substituted C₁₋₃₀alkyl, optionally substituted C₁-C₃₀alkoxy, C₁₄haloalkyl, optionally substituted C₂₋₄alkenyl, optionally substituted C₂₋₄alkynyl, optionally substituted C₁₋₃₀alkyl-CO₂H, or a nitrogen-protecting group; X^{L2} is O or S; Y^{L3} is O, S, NH, or a bond; R^{L3B} is H or optionally substituted alkyl. In some embodiments, L³ is —O—.

In some embodiments of any one of the aspects described herein, L′ is C₁₋₃₀alkylene, C₂₋₃₀alkenylene, C₂₋₃₀alkynylene, *-L³-C₁₋₃₀alkylene *-L³-C₂₋₃₀alkenylene, or *-L³-C₂₋₃₀alkynylene, where * is bond to R^{H} and L³ is R^{L3} is hydrogen, optionally substituted C₁₋₃₀alkyl, optionally substituted C₁-C₃₀alkoxy, C₁₋₄haloalkyl, optionally substituted C₂₋₄alkenyl, optionally substituted C₂-4alkynyl, optionally substituted C₁₋₃₀alkyl-CO₂H, or a nitrogen-protecting group; X^{L2} is O or S; Y^{L3} is O, S, NH, or a bond; R^{L3B} is H or optionally substituted alkyl.

In some embodiments of the various aspects described herein, L′ is a bond, —O— or an optionally substituted alkylene. For example, L¹ is —O— or —(CH₂)ₙ—, where n is 0 or an integer selected from 1 to 20 (e.g., n is 1, 2, 3, 4, 5, 6, 7, 8, 9, 10, 11, 12, 13, 14, or 15, such as n is 1, 2, 3, 4, 5 or 6).

In some embodiments of any one of the aspects described herein, L′ is —O—. In some other embodiments of any one of the aspects described herein, L′ is methylene, i.e., —CH₂—. In still some other embodiments of any one of the aspects described herein L′ is a bond.

In some embodiments of any one of the aspects described herein, L′ is

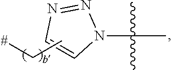

where b′ is 0 or integer from 1 to 20 (e.g., b′ is 0, 1, 2, 3, 4, 5 or 6); and # is a bond to R^{H}. For example, L¹ is

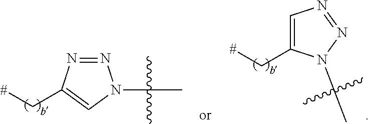

### L²

In some embodiment of any one of the aspects described herein, L² is a linker. For example, L² can be a direct bond or an atom such as oxygen or sulfur, a unit such as NR¹, C(O), C(O) O, C(O)NR¹, SO, SO₂, SO₂NH or a chain of atoms, such as substituted or unsubstituted alkyl, substituted or unsubstituted alkenyl, or substituted or unsubstituted alkynyl, where one or more methylenes can be interrupted or terminated by O, S, S(O), SO₂, N(R^{LL})₂, C(O), cleavable linking group, substituted or unsubstituted aryl, substituted or unsubstituted heteroaryl, substituted or unsubstituted heterocyclic; where R^{LL} is hydrogen, acyl, aliphatic or substituted aliphatic

In some embodiments of any one of the aspects described herein, L² is a bond or an optionally substituted alkylene. For example, L² is a bond. In some other non-limiting examples, L² is substituted or unsubstituted alkyl, substituted or unsubstituted alkenyl, or substituted or unsubstituted alkynyl, where one or more methylenes can be interrupted or terminated by O, S, S(O), SO₂, N(R^{LL})₂, C(O), cleavable linking group, substituted or unsubstituted aryl, substituted or unsubstituted heteroaryl, substituted or unsubstituted heterocyclic. In some embodiments, L² is —Z—(CH₂)ₘ—, where Z is absent, aryl, heteroaryl, cycloalkyl or heterocyclyl; and m is 0 or an integer selected from 1 to 20 (e.g., m is 1, 2, 3, 4, 5, 6, 7, 8, 9, 10, 11, 12, 13, 14 or 15, such as m is 1, 2, 3, 4, 5 or 6). For example, L² is —(CH₂)ₘ— or —(CH₂)ₘ-phenyl-.

### L³

In embodiments of the various aspects described herein, L³ can be —O—, —N(R^{L3})—, —S—, —C(O)—, —S(O)—, —S(O)₂—, —P(X^{L3})(Y^{L3}R^{L3B})—. For example, L³ can be —O— in some embodiments of the any one of the aspects described herein. In some embodiments of anyone of the aspects described herein, L³ can be —N(R^{L3})—, —S—, —C(O)—, —S(O)— or —S(O)₂—. In yet some other embodiments of any one of the aspects described herein, L³ can be —N(R^{L3})—, —S— or —C(O)—. In still some other embodiments of any one of the aspects described herein L³ can be —P(X^{L3})(Y^{L3}R^{L3B})—.

### R^{H}

In some embodiments of any one of the aspects described herein, R^{H} is an optionally substituted 6-membered heterocyclyl comprising a nitrogen atom and 0, 1 or 2 additional

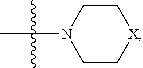

heteroatoms selected independently from N, O and S. For example, R^{H} is where X is O, NR^{L}, S, or CH₂; and R^{L} is hydrogen, a ligand, a linker covalently bonded to one or more ligands, aliphatic and aromatic alkyl, alkylester, alkylamine, dimethylamino alkyl, alkylether, alkylthioether, heteroaromatic alkyl, allyl, vinyl, alkyl groups functionalized with disulfide, oxime, ketone, acetal, hemiacetal, cleavable peptides, or cleavable sugars.

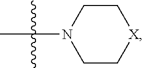

In some embodiments of any one of the aspects described herein, R^{H} is where X is O.

In some other embodiments of any one of the aspects described herein, R^{H} is

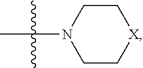

where X is NR^{L}. In some further embodiments, R^{L} is H or aliphatic and aromatic alkyl, alkylester, alkylamine, dimethylamino alkyl, alkylether, alkylthioether, heteroaromatic alkyl, allyl, vinyl, alkyl groups functionalized with disulfide, oxime, ketone, acetal, hemiacetal, cleavable peptides, or cleavable sugars. In yet some other embodiments of any one of the aspects described herein, R^{H} is a ligand or linker covalently bonded to one or more independently selected ligands.

In some embodiments of any one of the aspects described herein, R^{H} is

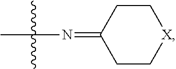

where X is O.

In some other embodiments of any one of the aspects described herein, R^{H} is

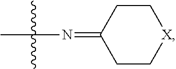

where X is NR^{L}. In some further embodiments, R^{L} is H or aliphatic and aromatic alkyl, alkylester, alkylamine, dimethylamino alkyl, alkylether, alkylthioether, heteroaromatic alkyl, allyl, vinyl, alkyl groups functionalized with disulfide, oxime, ketone, acetal, hemiacetal, cleavable peptides, or cleavable sugars. In yet some other embodiments of any one of the aspects described herein, R^{L} is a ligand or linker covalently bonded to one or more independently selected ligands.

R^{H2}

In some embodiments of any one of the aspects described herein, R^{H2} is an optionally substituted 6-membered heterocyclyl comprising a nitrogen atom and 0, 1 or 2 additional

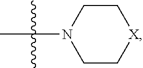

heteroatoms selected independently from N, O and S. For example, R^{H2} is where X is O, NR^{L}, S, or CH₂; and R^{L} is hydrogen, a ligand, a linker covalently bonded to one or more ligands, aliphatic and aromatic alkyl, alkylester, alkylamine, dimethylamino alkyl, alkylether, alkylthioether, heteroaromatic alkyl, allyl, vinyl, alkyl groups functionalized with disulfide, oxime, ketone, acetal, hemiacetal, cleavable peptides, or cleavable sugars.

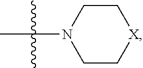

In some embodiments of any one of the aspects described herein, R^{H2} is where X is O.

In some other embodiments of any one of the aspects described herein, R^{H2} is

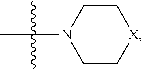

where X is NR^{L}. In some further embodiments, R^{L} is H or aliphatic and aromatic alkyl, alkylester, alkylamine, dimethylamino alkyl, alkylether, alkylthioether, heteroaromatic alkyl, allyl, vinyl, alkyl groups functionalized with disulfide, oxime, ketone, acetal, hemiacetal, cleavable peptides, or cleavable sugars. In yet some other embodiments of any one of the aspects described herein, R^{L} is a ligand or linker covalently bonded to one or more independently selected ligands.

In some embodiments of any one of the aspects described herein, one of R¹³ and R¹⁴ is an optionally substituted C₁-C₆alkyl. For example, one of R¹³ and R¹⁴ is methyl.

In some embodiments of any one of the aspects described herein, one of R¹³ and R¹⁴ is -L²-R^{H2} and the other is an optionally substituted C₁-C₆alkyl (e.g., methyl).

In some embodiments of any one of the aspects described herein, one of R¹³ and R¹⁴ is

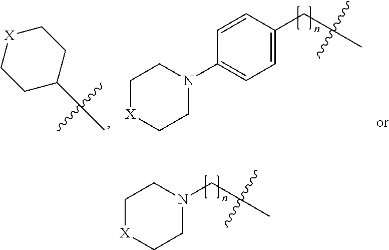

and the other of R¹³ and R¹⁴ is C₁-C₆alkyl,

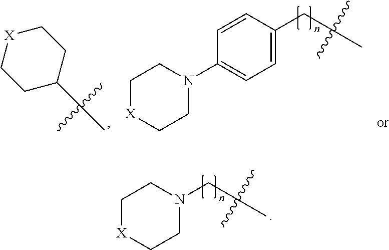

### R^{L}

In embodiments of the various aspects described herein, R^{L} can be hydrogen, a ligand, a linker covalently bonded to one or more ligands, aliphatic and aromatic alkyl, alkylester, alkylamine, dimethylamino alkyl, alkylether, alkylthioether, heteroaromatic alkyl, allyl, vinyl, alkyl groups functionalized with disulfide, oxime, ketone, acetal, hemiacetal, cleavable peptides, or cleavable sugars.

In some embodiments of any one of the aspects described herein, R^{L} is hydrogen, a ligand, a linker covalently bonded to one or more ligands, or optionally substituted aliphatic. For example, R^{L} is a ligand, a linker covalently bonded to one or more ligands.

In some embodiments of any one of the aspects described herein, R^{L} is -L⁴-L^{R}, where L⁴ is a linker and L^{R} is a ligand, a linker covalently bonded to one or more ligands, aliphatic and aromatic alkyl, alkylester, alkylamine, dimethylamino alkyl, alkylether, alkylthioether, heteroaromatic alkyl, allyl, vinyl, alkyl groups functionalized with disulfide, oxime, ketone, acetal, hemiacetal, cleavable peptides, or cleavable sugars. For example, L^{R} is a ligand.

In some embodiments of any one of the aspects described herein, L^{R} is aliphatic and aromatic alkyl, alkylester, alkylamine, dimethylamino alkyl, alkylether, alkylthioether, heteroaromatic alkyl, allyl, vinyl, alkyl groups functionalized with disulfide, oxime, ketone, acetal, hemiacetal, cleavable peptides, or cleavable sugars. For example, L^{R} is C₁₋₃₀alkyl, C₂₋₃₀alkenyl, C₂₋₃₀alkynyl, lipid, carbohydrate, folic acid, DUPA, RGD peptide, antibody, antibody fragment, peptide or other ligand.

In some embodiments of any one of the aspects described herein, L⁴ can be a direct bond or an atom such as oxygen or sulfur, a unit such as NRLL, C(O), C(O)O, C(O)NR¹, SO, SO₂, SO₂NH or a chain of atoms, such as substituted or unsubstituted alkyl, substituted or unsubstituted alkenyl, or substituted or unsubstituted alkynyl, where one or more methylenes can be interrupted or terminated by O, S, S(O), SO₂, N(R^{LL})₂, C(O), cleavable linking group, substituted or unsubstituted aryl, substituted or unsubstituted heteroaryl, substituted or unsubstituted heterocyclic; where R^{LL} is hydrogen, acyl, aliphatic or substituted aliphatic

In some embodiments of any one of the aspects described herein, L⁴ is a bond or an optionally substituted alkylene. For example, L⁴ is a bond. In some other non-limiting examples, L⁴ is substituted or unsubstituted alkyl, substituted or unsubstituted alkenyl, or substituted or unsubstituted alkynyl, where one or more methylenes can be interrupted or terminated by O, S, S(O), SO₂, N(R^{LL})₂, C(O), cleavable linking group, substituted or unsubstituted aryl, substituted or unsubstituted heteroaryl, substituted or unsubstituted heterocyclic. In some embodiments, L⁴ is —Z—(CH₂)ₘ—, where Z is absent, aryl, heteroaryl, cycloalkyl or heterocyclyl; and m is 0 or an integer selected from 1 to 20 (e.g., m is 1, 2, 3, 4, 5, 6, 7, 8, 9, 10, 11, 12, 13, 14 or 15, such as m is 1, 2, 3, 4, 5 or 6). For example, L⁴ is —(CH₂)ₘ— or —(CH₂)ₘ-phenyl-.

In some embodiments of any one of the aspects described herein, L⁴ comprises

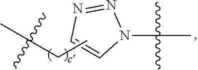

where c′ is 0 or an integer from 1 to 20 (e.g., d′ is 0, 1, 2, 3, 4, 5 or 6). For

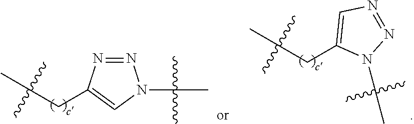

example, L⁴ comprises is 1.

In some embodiments, c′

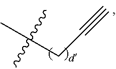

In some embodiments of any one of the aspects described herein, R^{L} is where d′ is 0 or an integer from 1 to 20 (e.g., d′ is 0, 1, 2, 3, 4, 5 or 6). In some embodiments d′ is 1.

In some embodiments of any one of the aspects described herein, R^{L} is

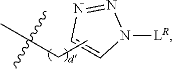

where d′ is 0 or an integer from 1 to 20 (e.g., d′ is 0, 1, 2, 3, 4, 5 or 6). For R^{L} is

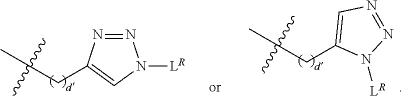

In some embodiments, d′ is 1.

In some embodiments of any one of the aspects described herein, R^{L} is —C(O)-L^{R}.

In some embodiment of any one of the aspects described herein, R^{L} is a nitrogen protecting group.

### B (Nucleobase)

In embodiments of the various aspects described herein, B is an optionally modified nucleobase. It is noted that the nucleobase can be a natural or non-natural nucleobase. By a “non-natural nucleobase” is meant a nucleobase other than adenine, guanine, cytosine, uracil, or thymine. Exemplary non-natural nucleobases include, but are not limited to, inosine, xanthine, hypoxanthine, nubularine, isoguanisine, tubercidine, and substituted or modified analogs of adenine, guanine, cytosine and uracil, such as 2-aminoadenine and other alkyl derivatives of adenine and guanine, 2-propyl and other alkyl derivatives of adenine and guanine, 5-halouracil and cytosine, 5-propynyl uracil and cytosine, 6-azo uracil, cytosine and thymine, 5-uracil (pseudouracil), 4-thiouracil, 5-halouracil, 5-(2-aminopropyl) uracil, 5-amino allyl uracil, 8-halo, amino, thiol, thioalkyl, hydroxyl and other 8-substituted adenines and guanines, 5-trifluoromethyl and other 5-substituted uracils and cytosines, 7-methylguanine, 5-substituted pyrimidines, 6-azapyrimidines and N-2, N-6 and O-6 substituted purines, including 2-aminopropyladenine, 5-propynyluracil and 5-propynylcytosine, dihydrouracil, 3-deaza-5-azacytosine, 2-aminopurine, 5-alkyluracil, 7-alkylguanine, 5-alkyl cytosine,7-deazaadenine, N6, N6-dimethyladenine, 2,6-diaminopurine, 5-amino-allyl-uracil, N3-methyluracil, substituted 1,2,4-triazoles, 2-pyridinone, 5-nitroindole, 3-nitropyrrole, uracil-5-oxyacetic 5-5-methoxyuracil, acid, methoxycarbonylmethyluracil, 5-methyl-2-thiouracil, 5-methoxycarbonylmethyl-2-thiouracil, 5-methylaminomethyl-2-thiouracil, 3-(3-amino-3carboxypropyl) uracil, 3-methylcytosine, 5-methylcytosine, N4-acetyl cytosine, 2-thiocytosine, N6-methyladenine, N6-isopentyladenine, 2-methylthio-N6-isopentenyladenine, N-methylguanines, or O-alkylated bases. Further purines and pyrimidines include those disclosed in U.S. Pat. No. 3,687,808, those disclosed in the Concise Encyclopedia of Polymer Science and Engineering, pages 858-859, Kroschwitz, J. I., ed. John Wiley & Sons, 1990, and those disclosed by Englisch et al., Angewandte Chemie, International Edition, 1991, 30, 613, content of all which is incorporated herein by reference.

In some embodiments, the non-natural nucleobase can be selected from the group consisting of inosine, xanthine, hypoxanthine, nubularine, isoguanisine, tubercidine, 2-(halo) adenine, 2-(alkyl) adenine, 2-(propyl) adenine, 2-(amino) adenine, 2-(aminoalkyll) adenine, 2-(aminopropyl) adenine, 2-(methylthio)-N6-(isopentenyl) adenine, 7-(deaza) adenine, 8-(alkenyl) adenine, 8-(alkyl) adenine, 8-(alkynyl) adenine, 8-(amino) adenine, 8-(halo) adenine, 8-(hydroxyl) adenine, 8-(thioalkyl) adenine, 8-(thiol) adenine, N6-(isopentyl) adenine, N6-(methyl) adenine, N°, N6-(dimethyl) adenine, 2-(alkyl) guanine,2-(propyl) guanine, 6-(alkyl) guanine, 6-(methyl) guanine, 7-(alkyl) guanine, 7-(methyl) guanine, 7-(deaza) guanine, 8-(alkyl) guanine, 8-(alkenyl) guanine, 8-(alkynyl) guanine, 8-(amino) guanine, 8-(halo) guanine, 8-(hydroxyl) guanine, 8-(thioalkyl) guanine, 8-(thiol) guanine, N-(methyl) guanine, 2-(thio) cytosine, 3-(deaza)-5-(aza) cytosine, 3-(alkyl) cytosine, 3-(methyl) cytosine, 5-(alkyl) cytosine, 5-(alkynyl) cytosine, 5-(halo) cytosine, 5-(methyl) cytosine, 5-(propynyl) cytosine, 5-(propynyl) cytosine, 5-(trifluoromethyl) cytosine, 6-(azo) cytosine, N4-(acetyl) cytosine, 3-(3-amino-3-carboxypropyl) uracil, 2-(thio) uracil, 5-(methyl)-2-(thio) uracil, 5-(methylaminomethyl)-2-(thio) uracil, 4-(thio) uracil, 5-(methyl)-4-(thio) uracil, 5-(methylaminomethyl)-4-(thio) uracil, 5-(methyl)-2,4-(dithio) uracil, 5-(methylaminomethyl)-2,4-(dithio) uracil, 5-(2-aminopropyl) uracil, 5-(alkyl) uracil, 5-(alkynyl) uracil, 5-(allylamino) uracil, 5-(aminoallyl) uracil, 5-(aminoalkyl) uracil, 5-(guanidiniumalkyl) uracil, 5-(1,3-diazole-1-alkyl) uracil, 5-(cyanoalkyl) uracil, 5-(dialkylaminoalkyl) uracil, 5-(dimethylaminoalkyl) uracil, 5-(halo) uracil, 5-(methoxy) uracil, uracil-5-oxyacetic acid, 5-(methoxycarbonylmethyl)-2-(thio) uracil, 5-(methoxycarbonyl-methyl) uracil, 5-(propynyl) uracil, 5-(propynyl) uracil, 5-(trifluoromethyl) uracil, 6-(azo) uracil, dihydrouracil, N³-(methyl) uracil, 5-uracil (i.e., pseudouracil), 2-(thio) pseudouracil,4-(thio) pseudouracil,2,4-(dithio) psuedouracil,5-(alkyl) pseudouracil, 5-(methyl) pseudouracil, 5-(alkyl)-2-(thio) pseudouracil, 5-(methyl)-2-(thio) pseudouracil, 5-(alkyl)-4-(thio) pseudouracil, 5-(methyl)-4-(thio) pseudouracil, 5-(alkyl)-2,4-(dithio) pseudouracil, 5-(methyl)-2,4-(dithio) pseudouracil, 1-substituted pseudouracil, 1-substituted 2 (thio)-pseudouracil, 1-substituted 4-(thio) pseudouracil, 1-substituted 2,4-(dithio) pseudouracil, 1-(aminocarbonylethylenyl)-pseudouracil, 1-(aminocarbonylethylenyl)-2 (thio)-pseudouracil, 1-(aminocarbonylethylenyl)-4-(thio) pseudouracil, 1-(aminocarbonylethylenyl)-2,4-(dithio) pseudouracil, 1-(aminoalkylaminocarbonylethylenyl)-pseudouracil, 1-(aminoalkylamino-carbonylethylenyl)-2 (thio)-pseudouracil, 1-(aminoalkylaminocarbonylethylenyl)-4-(thio) pseudouracil, 1-(aminoalkylaminocarbonylethylenyl)-2,4-(dithio) pseudouracil, 1,3-(diaza)-2-(oxo)-phenoxazin-1-yl, 1-(aza)-2-(thio)-3-(aza)-phenoxazin-1-yl, 1,3-(diaza)-2-(oxo)-phenthiazin-1-yl, 1-(aza)-2-(thio)-3-(aza)-phenthiazin-1-yl, 7-substituted 1,3-(diaza)-2-(oxo)-phenoxazin-1-yl, 7-substituted 1-(aza)-2-(thio)-3-(aza)-phenoxazin-1-yl, 7-substituted 1,3-(diaza)-2-(oxo)-phenthiazin-1-yl, 7-substituted 1-(aza)-2-(thio)-3-(aza)-phenthiazin-1-yl, 7-(aminoalkylhydroxyl)-1,3-(diaza)-2-(oxo)-phenoxazin-1-yl, 7-(aminoalkylhydroxyl)-1-(aza)-2-(thio)-3-(aza)-phenoxazin-1-yl, 7-(aminoalkylhydroxyl)-1,3-(diaza)-2-(oxo)-phenthiazin-1-yl, 7-(aminoalkylhydroxyl)-1-(aza)-2-(thio)-3-(aza)-phenthiazin-1-yl, 7-(guanidiniumalkylhydroxyl)-1,3-(diaza)-2-(oxo)-phenoxazin-1-yl, 7-(guanidiniumalkylhydroxyl)-1-(aza)-2-(thio)-3-(aza)-phenoxazin-1-yl, 7-(guanidiniumalkyl-hydroxyl)-1,3-(diaza)-2-(oxo)-phenthiazin-1-yl, 7-(guanidiniumalkylhydroxyl)-1-(aza)-2-(thio)-3-(aza)-phenthiazin-1-yl, 1,3,5-(triaza)-2,6-(dioxa)-naphthalene, inosine, xanthine, hypoxanthine, nubularine, tubercidine, isoguanisine, inosinyl, 2-aza-inosinyl, 7-deaza-inosinyl, nitroimidazolyl, nitropyrazolyl, nitrobenzimidazolyl, nitroindazolyl, aminoindolyl, pyrrolopyrimidinyl, 3-(methyl) isocarbostyrilyl, 5-(methyl) isocarbostyrilyl, 3-(methyl)-7-(propynyl) isocarbostyrilyl, 7-(aza) indolyl, 6-(methyl)-7-(aza) indolyl, imidizopyridinyl, 9-(methyl)-imidizopyridinyl, pyrrolopyrizinyl, isocarbostyrilyl, 7-(propynyl) isocarbostyrilyl, propynyl-7-(aza) indolyl, 2,4,5-(trimethyl)phenyl, 4-(methyl) indolyl, 4,6-(dimethyl) indolyl, phenyl, napthalenyl, anthracenyl, phenanthracenyl, pyrenyl, stilbenyl, tetracenyl, pentacenyl, difluorotolyl, 4-(fluoro)-6-(methyl)benzimidazole, 4-(methyl)benzimidazole, 6-(azo) thymine, 2-pyridinone, 5-nitroindole, 3-nitropyrrole, 6-(aza)pyrimidine, 2-(amino) purine, 2,6-(diamino) purine, 5-substituted pyrimidines, N²-substituted purines, N⁶-substituted purines, 06-substituted purines, substituted 1,2,4-triazoles, and any O-alkylated or N-alkylated derivatives thereof.

In some embodiments, a non-natural nucleobase is a modified nucleobase, i.e., the nucleobase comprises a nucleobase modification described herein, e.g., the nucleobase is a substituted or modified analog of any of the natural nucleobases. Examples of the nucleobase modifications include, but not limited to: C-5 pyrimidine with an alkyl group or aminoalkyls and other cationic groups such as guanidinium and amidine functionalities, N²- and N⁶- with an alkyl group or aminoalkyls and other cationic groups such as guanidinium and amidine functionalities of purines, G-clamps, guanidinium G-clamps, and pseudouridine known in the art.

In some embodiments of any one of the aspects, the non-natural nucleobase is a universal nucleobase. As used herein, a universal nucleobase is any modified or unmodified natural or non-natural nucleobase that can base pair with all of adenine, cytosine, guanine and uracil without substantially affecting the melting behavior, recognition by intracellular enzymes or activity of the oligonucleotide comprising the universal nucleobase. Some exemplary universal nucleobases include, but are not limited to, 2,4-difluorotoluene, nitropyrrolyl, nitroindolyl, 8-aza-7-deazaadenine, 4-fluoro-6-methylbenzimidazle, 4-methylbenzimidazle, 3-methyl isocarbostyrilyl, 5-methyl isocarbostyrilyl, 3-methyl-7-propynyl isocarbostyrilyl, 7-azaindolyl, 6-methyl-7-azaindolyl, imidizopyridinyl, 9-methyl-imidizopyridinyl, pyrrolopyrizinyl, isocarbostyrilyl, 7-propynyl isocarbostyrilyl, propynyl-7-azaindolyl, 2,4,5-trimethylphenyl, 4-methylinolyl, 4,6-dimethylindolyl, phenyl, napthalenyl, anthracenyl, phenanthracenyl, pyrenyl, stilbenyl, tetracenyl, pentacenyl, and structural derivatives thereof.

In some embodiments of any one of the aspects described herein, the non-matural nucleobase is a protected nucleobase. As used herein, a “protected nucleobase” refers to a nucleobase comprising a nitrogen protecting group, and/or an oxygen protecting group, and/or a sulfur protecting group.

In some embodiments of any one of the aspects described herein, the non-natural nucleobase is a modified, protected or substituted analogs of a nucleobase selected from adenine, cytosine, guanine, thymine, and uracil.

In some embodiments of any one of the aspects described herein, the nucleobase is a pyrimidine modified at the C₄ position.

In some embodiments of any one of the aspects described herein, the nucleobase is a pyrimidine modified at the C₅ position.

In some embodiments of any one of the aspects described herein, the nucleobase is a purine modified at the N2 position. In some embodiments of any one of the aspects described herein, the nucleobase is a purine modified at the N6 position.

In some embodiments of any one of the aspects described herein, the nucleobase is a purine modified at the C₆ position.

In some embodiments of any one of the aspects described herein, the nucleobase is a N-7 deaza purine, optionally modified at the N7 position.

### Double-Stranded RNAs

The skilled person is well aware that double-stranded RNAs comprising a duplex structure of between 20 and 23, but specifically 21, base pairs have been hailed as particularly effective in inducing RNA interference (Elbashir et al., EMBO 2001, 20:6877-6888). However, others have found that shorter or longer double-stranded oligonucleotides can be effective as well.

Accordingly, in one aspect, provided herein is a double-stranded RNA (dsRNA) comprising a first strand (also referred to as an antisense strand or a guide strand) and a second strand (also referred to as a sense strand or passenger strand, wherein at least one of the first (i.e., the antisense strand) or the second strand (i.e., the sense strand) is an oligonucleotide described herein. In other words, at least one of the first (i.e., the antisense strand) or the second strand (i.e., the sense strand) comprises at least one nucleotide of Formula (II).

In some embodiments of any one of the aspects described herein, the sense strand is an oligonucleotide described herein. In other words, the sense strand comprises at least one nucleotide of Formula (II). In some embodiments of any one of the aspects described herein, the antisense strand is an oligonucleotide described herein. In other words, the antisense strand comprises at least one nucleotide of Formula (II). Preferably, the sense strand comprises at least one nucleotide of Formula (II).

In some embodiments of the various aspects described herein, the antisense strand is substantially complementary to a target nucleic acid, e.g., a target gene or mRNA gene and the dsRNA is capable of inducing targeted cleavage of the target nucleic acid.

For the dsRNA molecules to be more effective in vivo, the antisense strand must have some metabolic stability. In other words, for the dsRNA molecules to be more effective in vivo, some amount of the antisense stand may need to be present in vivo after a period time after administration. Accordingly, in some embodiments, at least 40%, for example at least 45%, at least 50%, at least 55%, at least 60%., at least 65%, at least 70%, at least 75%, or at least 80% of the antisense strand of the dsRNA is present in vivo, for example in mouse liver, at day 5 after in vivo administration. In some embodiments, at least 40%, for example at least 45%, at least 50%, at least 55%, at least 60%., at least 65%, at least 70%, at least 75%, or at least 80% of the antisense strand of the dsRNA is present in vivo, for example in mouse liver, at day 6 after in vivo administration. In some embodiments, at least 40%, for example at least 45%, at least 50%, at least 55%, at least 60%, at least 65%, at least 70%, at least 75%, or at least 80% of the antisense strand of the dsRNA is present in vivo, for example in mouse liver, at day 7 after in vivo administration. In some embodiments, at least 40%, for example at least 45%, at least 50%, at least 55%, at least 60%, at least 65%, at least 70%, at least 75%, or at least 80% of the antisense strand of the dsRNA is present in vivo, for example in mouse liver, at day 8 after in vivo administration. In some embodiments, at least 40%, for example at least 45%, at least 50%, at least 55%, at least 60%., at least 65%, at least 70%, at least 75%, or at least 80% of the antisense strand of the dsRNA is present in vivo, for example in mouse liver, at day 9 after in vivo administration. In some embodiments, at least 40%, for example at least 45%, at least 50%, at least 55%, at least 60%., at least 65%, at least 70%, at least 75%, or at least 80% of the antisense strand of the dsRNA is present in vivo, for example in mouse liver, at day 10 after in vivo administration. In some embodiments, at least 40%, for example at least 45%, at least 50%, at least 55%, at least 60%., at least 65%, at least 70%, at least 75%, or at least 80% of the antisense strand of the dsRNA is present in vivo, for example in mouse liver, at day 11 after in vivo administration. In some embodiments, at least 40%, for example at least 45%, at least 50%, at least 55%, at least 60%., at least 65%, at least 70%, at least 75%, or at least 80% of the antisense strand of the dsRNA is present in vivo, for example in mouse liver, at day 12 after in vivo administration. In some embodiments, at least 40%, for example at least 45%, at least 50%, at least 55%, at least 60%., at least 65%, at least 70%, at least 75%, or at least 80% of the antisense strand of the dsRNA is present in vivo, for example in mouse liver, at day 13 after in vivo administration. In some embodiments, at least 40%, for example at least 45%, at least 50%, at least 55%, at least 60%., at least 65%, at least 70%, at least 75%, or at least 80% of the antisense strand of the dsRNA is present in vivo, for example in mouse liver, at day 14 after in vivo administration. In some embodiments, at least 40%, for example at least 45%, at least 50%, at least 55%, at least 60%., at least 65%, at least 70%, at least 75%, or at least 80% of the antisense strand of the dsRNA is present in vivo, for example in mouse liver, at day 15 after in vivo administration.

### Strand Lengths

Embodiments of the various aspects described herein include a double-stranded nucleic acid, e.g., dsRNA comprising an antisense strand and a sense strand. It is noted that each strand can range from 12-40 nucleotides in length. For example, each strand independently can be between 14-40 nucleotides in length, 17-37 nucleotides in length, 25-37 nucleotides in length, 27-35 nucleotides in length, 17-23 nucleotides in length, 17-21 nucleotides in length, 17-19 nucleotides in length, 19-25 nucleotides in length, 19-23 nucleotides in length, 19-21 nucleotides in length, 21-25 nucleotides in length, 21-23 nucleotides in length, 25-35 nucleotides in length, 26-35 nucleotides in length, 27-34 nucleotides in length, 28-32 nucleotides in length or 29-31 nucleotides in length. Without limitations, the sense and antisense strands can be equal length or unequal length. In some embodiments, the antisense strand is longer, e.g., by 1, 2, 3, 4, or 5 nucleotides than the sense strand.

In some embodiments, the antisense strand is of length 18 to 35 nucleotides. In some embodiments, the antisense strand is 21-25, 19-25, 19-21, 21-23 nucleotides in length. In some embodiments, the antisense strand is 21, 22, 23, 24, 25, 26, 27, 28, 29, 30, 31 or 32 nucleotides in length. In some embodiments, the antisense strand is 21, 22, 23, 24, or 25 nucleotides in length. In some preferred embodiments, the antisense strand is 22, 23 or 25 nucleotides in length.

Similar to the antisense strand, the sense strand can be, in some embodiments, 18-35 nucleotides in length. In some embodiments, the sense strand is 21-25, 19-25, 19-21 or 21-23 nucleotides in length. In some embodiments, the antisense strand is 19, 20, 21, 22, 23, 24, 25, 26, 27, 28 or 29 nucleotides in length. In some embodiments, the antisense strand is 19, 21, 22 or 23 nucleotides in length. In some preferred embodiments, the sense strand is 21 nucleotides in length.

In some embodiments of any one of the aspects described herein, the sense strand is 15 nucleotides in length and the antisense strand is 18, 19, 20, 21, or 22 (e.g., 20) nucleotides in length. In some embodiments of any one of the aspects described herein, the sense strand is 19 nucleotides in length and the antisense strand is 19, 20, or 21 nucleotides in length. In some embodiments of any one of the aspects described herein, the sense strand is 20 nucleotides in length and the antisense strand is 20, 21, or 22 nucleotides in length. In some embodiments of any one of the aspects described herein, the sense strand is 21 nucleotides in length and the antisense strand is 21, 22, or 23 nucleotides in length. In some embodiments of any one of the aspects described herein, the sense strand is 20-24 (e.g., 22) nucleotides in length and the antisense strand is 34-38 (e.g. 36) nucleotides in length.

In some embodiments, the antisense strand is 21, 22 or 25 nucleotides in length and the sense strand is 21 nucleotides in length.

### Double-Stranded Region

The sense strand and antisense strand typically form a double-stranded or duplex region. Generally, the duplex region (double-stranded region) is 12-40 nucleotide base pairs in length, 15-35 nucleotide base pairs in length, 17-30 nucleotide base pairs in length, 25-35 nucleotides base pairs in length, 27-35 nucleotide base pairs in length, 17-23 nucleotide base pairs in length, 17-21 nucleotide base pairs in length, 17-19 nucleotide base pairs in length, 19-25 nucleotide pairs in length, 19-23 nucleotide base pairs in length, 19-21 nucleotide base pairs in, 21-25 nucleotide base pairs in length, or 21-23 nucleotide base pairs in length. For example, the dsRNA has a duplex region of 15-35 nucleotide pairs in length. In some embodiments, the dsRNA has a duplex region of 18, 19, 20, 21, 22, 22, 23, 24, 25, 26, 27, 28, 29, 30 or 31 nucleotide base pairs in length. In some embodiments, the dsRNA has a duplex region of 19, 20, 21, 22 or 23 nucleotide base pairs in length. In some preferred embodiments, the dsRNA has a duplex region of 21 nucleotide base pairs in length.

### Overhangs

In some embodiments, the dsRNA comprises one or more overhang regions (i.e., single-stranded region) and/or capping groups of strands at the 3′-end, or 5′-end, or both ends of a strand. Without limitations, the overhang can be 1-10 nucleotides in length, 1-6 nucleotides in length, 1-5 nucleotides in length, 1-4 nucleotides in length, 1-3 nucleotides in length, 2-6 nucleotides in length, 2-5 nucleotides in length 2-4 nucleotides in length, 2-3 nucleotides in length, or 1-2 nucleotides in length. The overhangs can be the result of one strand being longer than the other, or the result of two strands of the same length being staggered. The overhang can form a mismatch with the sequence being targeted or it can be complementary to the sequence being targeted or can be other sequence. The first and second strands can also be joined, e.g., by additional bases to form a hairpin, or by other non-base linkers. Without limitations the overhang can be present at the 3′-end of the sense strand, antisense strand or both strands.

In some embodiments, the dsRNA comprises a single overhang. For example, the dsRNA has a single overhang and the overhang is at least two, three, four, five, six, seven, eight, nine, or ten nucleotides in length. In some embodiments, the overhang is present at the 3′-end of the antisense strand. In some particular embodiments, the dsRNA comprises a two nucleotide overhang at the 3′-end of the antisense strand.

The dsRNA can also have a blunt end. For example, one end of the dsRNA is a blunt end and the other end has an overhang. Without limitations, the blunt end can be located at the 5′-end of the antisense strand (or the 3′-end of the sense strand) or vice versa. Generally, the antisense strand of the dsRNA has a nucleotide overhang at the 3′-end, and the 5′-end is blunt. In some embodiments, the dsRNA has a 2 nucleotide overhang on the 3′-end of the antisense strand and a blunt end at the 5′-end of the antisense strand.

In some other embodiments, the dsRNA has two blunt ends, i.e., at both ends of the dsRNA.

The nucleotides in the overhang region can each independently be a modified or unmodified nucleotide including, but not limited to 2′-sugar modified, such as, 2′-fluoro, 2′-O-methyl, thymidine (T), 2′-O-methoxyethyl-5-methyluridine, 2′-O-methoxyethyladenosine, 2′-O-methoxyethyl-5-methylcytidine, GNA, SNA, hGNA, hhGNA, mGNA, TNA, h′GNA, and any combinations thereof. For example, TT (or UU) can be an overhang sequence for either end on either strand. The 5′- or 3′-overhangs at the sense strand, antisense strand or both strands can be phosphorylated. In some embodiments, the overhang region contains two nucleotides having a phosphorothioate internucleotide linkage between the two nucleotides, where the two nucleotides in the overhang region can be the same or different.

The internucleoside linkages in the overhang region can be a modified or unmodified internucleotide linkage. For example, the overhang region can comprise one, e.g., two or more, phosphorothioate internucleoside linkages.

### Nucleic Acid Modifications

In some embodiments of any one of the aspects, the oligonucleotide or double-stranded nucleic acid described herein can comprise one or more nucleic acid modifications. For example, the oligonucleotide or double-stranded nucleic acid described herein can comprise at least one, e.g., e.g., two, three, four, five, six, seven, eight, nine, ten, eleven, twelve, thirteen, fourteen, fifteen or more nucleic acid modifications. It is noted that when two are more modifications are present, they can be same, different or some combination of same and different. Further, the modifications all can be present in one strand of the double-stranded nucleic acid. In some embodiments, both strands of the double-stranded nucleic acid comprise at least one nucleic acid modification. When both strands comprise at least one modification, the modifications can be same, different or some combination of same and different.

### 2′-Fluoro Modified Nucleotides

In some embodiments, the oligonucleotide or dsRNA described herein can further comprise 2′-fluoro nucleotides, i.e., 2′-fluoro modifications. For example, the oligonucleotide or dsRNA described herein can comprise at least one, e.g., two, three, four, five, six, seven, eight, nine, ten, eleven, twelve, thirteen, fourteen, fifteen or more 2′-fluoro nucleotides. It is noted that the 2′-fluoro nucleotides all can be present in one strand of the dsRNA.

The antisense strand can comprise at least one or more 2′-fluoro nucleotides. For example, the antisense strand can comprise at least two (e.g., two, three, four, five, six, seven, eight, nine, ten or more) further 2′-fluoro nucleotides. In some embodiments, the antisense strand comprises one, two, three, four, five or six 2′-fluoro nucleotides. Without limitations, the additional 2′-fluoro modification(s) in the antisense strand can be present at any position. In some embodiments, the antisense strand comprises at least three 2′-fluoro nucleotides. For example, the antisense strand comprises a 2′-fluoro nucleotide at least at positions 2, 14 and 16 from the 5′-end. In some other embodiments, the antisense comprises at least four 2′-fluoro nucleotides. For example, the antisense comprises a 2′-fluoro nucleotide at least at positions 2, 6, 14 and 16 from the 5′-end. In some further embodiments, the antisense strand comprises at least five 2′-fluoro nucleotides. For example, the antisense strand comprises a 2′-fluoro nucleotide at least at positions 2, 6, 9, 14 and 16 from the 5′-end. In still some further embodiments, the antisense strand comprises at least six 2′-fluoro nucleotides. For example, the antisense strand comprises a 2′-fluoro nucleotide at least at positions 2, 6, 8, 9, 14 and 16 from the 5′-end.

In some embodiments, the antisense strand comprises at least one 2′-fluoro nucleotide adjacent to a destabilizing modification. For example, the 2′-fluoro nucleotide can be the nucleotide at the 5′-end or the 3′-end of a destabilizing modification, i.e., at position-1 or +1 from the position of the destabilizing modification. In some embodiments, the antisense strand comprises a 2′-fluoro nucleotide at each of the 5′-end and the 3′-end of the destabilizing modification, i.e., positions-1 and +1 from the position of the destabilizing modification. In some embodiments, the antisense strand comprises at least two 2′-fluoro nucleotides at the 3′-end of the destabilizing modification, i.e., at positions+1 and +2 from the position of the destabilizing modification.

In some embodiments, the antisense strand does not comprise a 2′-fluoro nucleotide at positions 3-9, counting from 5′-end.

The sense strand can comprise at least one or more 2′-fluoro nucleotides. For example, the antisense strand can comprise at least two (e.g., two, three, four, five, six, seven, eight, nine, ten or more) 2′-fluoro nucleotides. In some embodiments, the sense strand comprises one, two, three, four, or five 2′-fluoro nucleotides. For example, the sense strand comprises three or four 2′-fluoro nucleotides. Without limitations, a 2′-fluoro modification in the sense strand can be present at any positions. In some embodiments, the sense strand comprises at least three 2′-fluoro nucleotides. For example, the sense comprises a 2′-fluoro nucleotide at least at positions 7, 9 and 11 from the 5′-end or at positions 11, 13 and 15, counting from the 3′-end. In some other embodiments, the sense strand comprises at least four 2′-fluoro nucleotides. For example, the sense comprises a 2′-fluoro nucleotide at least at positions 7, 9, 10 and 11 from the 5′-end or at positions 11, 12, 13 and 15, counting from the 3′-end. In some embodiments of any one of the aspects described herein, the sense strand comprises a 2′-fluoro nucleotide at positions 9, 10, and 11, counting from the 5′-end of the sense strand or at positions 11, 12, and 13 counting from the 3′-end of the sense strand. In some embodiments, the sense strand comprises a block of two, three or four 2′-fluoro nucleotides.

In some embodiments, the sense strand comprises 2′-fluoro nucleotides at positions opposite or complimentary to positions 11, 12 and 15 of the antisense strand, counting from the 5′-end of the antisense strand. In some other embodiments, the sense strand comprises 2′-fluoro nucleotides at positions opposite or complimentary to positions 11, 12, 13, and 15 of the antisense strand, counting from the 5′-end of the antisense strand.

In some embodiments, the sense strand comprises a block of two, three or four 2′-fluoro nucleotides.

In some embodiments, the sense strand does not comprise a 2′-fluoro nucleotide in position opposite or complimentary to a thermally destabilizing modification of the duplex in the antisense strand.

In some embodiments, both the sense and the antisense strands comprise at least one, e.g., at least two 2′-fluoro nucleotides. The 2′-fluoro modification can occur on any nucleotide of the sense strand or antisense strand. For instance, the 2′-fluoro modification can occur on every nucleotide on the sense strand and/or antisense strand; each 2′-fluoro modification can occur in an alternating pattern on the sense strand or antisense strand; or the sense strand and antisense strand both comprise 2′-fluoro modifications in an alternating pattern. The alternating pattern of the 2′-fluoro modifications on the sense strand can be the same or different from the antisense strand, and the alternating pattern of the 2′-fluoro modifications on the sense strand can have a shift relative to the alternating pattern of the 2′-fluoro modifications on the antisense strand.

### 2′-Deoxy (2′-H, DNA) Nucleotides

In some embodiments, the oligonucleotide or dsRNA described herein can further comprise 2′-deoxy (e.g., 2′-H or DNA) nucleotides. For example, the oligonucleotide or dsRNA described herein can comprise at least one, e.g., two, three, four, five, six, seven, eight, nine, ten, eleven, twelve, thirteen, fourteen, fifteen or more DNA nucleotides. It is noted that the DNA nucleotides all can be present in one strand in the dsRNA of the dsRNA.

In some embodiments, the antisense strand can comprise at least one (e.g., two, three, four, five, six, seven, eight, nine, ten or more) DNA nucleotides. In some embodiments, the antisense strand comprises two, three, four, five or six DNA nucleotides. Without limitations, the DNA nucleotides in the antisense strand can be present at any position. For example, the antisense strand comprises a 2′-deoxy nucleotide at 1, 2, 3, 4, 5 or 6 of positions 2, 5, 7, 12, 14 and 16, counting from 5′-end of the antisense strand. In one non-limiting example, the antisense strand comprises a 2′-deoxy nucleotide at 1, 2, 3 or 4 of positions 2, 5, 7, and 12, counting from 5′-end of the antisense strand.

In some embodiments, the antisense comprises a 2′-deoxy nucleotide at position 2 or 12, counting from 5′-end of the antisense strand. For example, the antisense comprises a 2′-deoxy nucleotide at position 12, counting from 5′-end of the antisense strand. In some embodiments, the antisense comprises a 2′-deoxy nucleotide at positions 5 and 7, counting from 5′-end of the antisense strand. For example, the antisense strand comprises a 2′-deoxy nucleotide at positions 5, 7 and 12, counting from 5′-end of the antisense strand. In some embodiments, the antisense strand comprises a 2′-deoxy nucleotide at positions 2, 5 and 7, counting from 5′-end of the antisense strand. In some other embodiments, the antisense comprises at least four DNA nucleotides. For example, the antisense comprises a DNA nucleotide at least at positions 2, 5, 7 and 12, counting from the 5′-end. In some further embodiments, the antisense strand comprises at least five DNA nucleotides. In still some further embodiments, the antisense strand comprises at least six DNA nucleotides. For example, the antisense strand comprises a DNA nucleotide at least at positions 2, 5, 7, 12, 14 and 16, counting from the 5′-end.

In some embodiments of any one of the aspects described herein, the antisense strand comprises a DNA nucleotide at positions 2, 5, 7, and 12 counting from the 5′-end of the antisense strand; and a 2′-fluoro nucleotide at position 14 of the antisense strand.

As described herein, the dsRNA can comprise at least one, e.g., at least two, at least three, at least four, at least five, at least six, at least seven or more, 2′-deoxy modifications in a central region of the sense strand and/or the antisense strand. For example, at least one of the sense stand and the antisense can comprise at least one, e.g., at least two, at least three, at least four, at least five, at least six, at least seven or more, 2′-deoxy modification in positions 5-17, e.g., positions 6-16, positions 6-15, positions 6-14, positions 6-13, positions 6-12, positions 7-15, positions 7-14, positions 7-13, positions, 7-12, positions 8-16, positions 8-15, positions 8-14, positions 8-13, positions 8-12, positions 9-16, positions 9-15, positions 9-14, positions 9-13, positions 9-12, positions 10-16, positions 10-15, positions 10-14, positions 10-13 or positions 10-12, counting from the 5′-end of the sense strand or the antisense strand.

In some embodiments, both the sense and the antisense strands comprise at least one DNA nucleotide. The DNA nucleotide can occur on any nucleotide of the sense strand or antisense strand. For instance, the DNA nucleotide can occur on every nucleotide on the sense strand and/or antisense strand; each DNA nucleotide can occur in an alternating pattern on the sense strand or antisense strand; or the sense strand and antisense strand both comprise DNA nucleotides in an alternating pattern. The alternating pattern of the DNA nucleotides on the sense strand can be the same or different from the antisense strand, and the alternating pattern of the DNA nucleotides on the sense strand can have a shift relative to the alternating pattern of the DNA nucleotides on the antisense strand.

In some embodiments, the dsRNA comprises at least three 2′-deoxy modifications, wherein the 2′-deoxy modifications are at positions 2 and 14 of the antisense strand, counting from 5′-end of the antisense strand, and at position 11 of the sense strand, counting from 5′-end of the sense strand.

In some embodiments, the dsRNA comprises at least five 2′-deoxy modifications, wherein the 2′-deoxy modifications are at positions 2, 12 and 14 of the antisense strand, counting from 5′-end of the antisense strand, and at positions 9 and 11 of the sense strand, counting from 5′-end of the sense strand.

In some embodiments, the dsRNA comprises at least seven 2′-deoxy modifications, wherein the 2′-deoxy modifications are at positions 2, 5, 7, 12 and 14 of the antisense strand, counting from 5′-end of the antisense strand, and at positions 9 and 11 of the sense strand, counting from 5′-end of the sense strand.

In one non-limiting example, the sense strand does not comprise a 2′-deoxy nucleotide at position 11, counting from 5′-end of the sense strand.

### 2′-OMe Nucleotides

In some embodiments, the oligonucleotide or dsRNA described herein can comprise at least one, e.g., one, two, three, four, five, six, seven, eight, nine, ten, eleven, twelve, thirteen, fourteen, fifteen, sixteen, seventeen, eighteen, nineteen, twenty or more 2′-OMe nucleotides. It is noted that the 2′-OMe nucleotides all can be present in one strand of the dsRNA.

In some embodiments, the antisense strand can comprise at least one, e.g., two, three, four, five, six, seven, eight, nine, ten, eleven, twelve, thirteen, fourteen, fifteen, sixteen, seventeen or more 2′-OMe nucleotides. Without limitations, a 2′-OMe nucleotide in the antisense strand can be present at any position. In some embodiments of any one of the aspects described herein, all remaining nucleotides in the antisense strand are 2′-OMe nucleotides.

In some embodiments, the antisense strand does not comprise 2′-OMe nucleotides at least at positions 2, 14 and 16 from the 5′-end. In some other embodiments, the antisense does not comprise 2′-OMe nucleotides at least at positions 2, 6, 14 and 16 from the 5′-end. In some further embodiments, the antisense strand does not comprise 2′-OMe nucleotides at least at positions 2, 6, 9, 14 and 16 from the 5′-end. In still some further embodiments, the antisense strand does not comprise 2′-OMe nucleotides at least at positions 2, 6, 8, 9, 14 and 16 from the 5′-end.

The sense strand can comprise at least one, e.g., two, three, four, five, six, seven, eight, nine, ten, eleven, twelve, thirteen, fourteen, fifteen, sixteen or more 2′-OMe nucleotides. Without limitations, a 2′-OMe nucleotide in the sense strand can be present at any positions. In some embodiments, all remaining nucleotides in the sense strand are 2′-OMe nucleotides.

In some embodiments, the sense does not comprise 2′-OMe nucleotides at least at positions 7, 10 and 11 from the 5′-end or at positions 11, 12 and 15, counting from the 3′-end. In some other embodiments, the sense does not comprise 2′-OMe nucleotides at least at positions 7, 9, 10 and 11 from the 5′-end or at positions 11, 12 13, and 15, counting from the 3′-end.

In some embodiments, both the sense and the antisense strands comprise at least one 2′-OMe nucleotide. The 2′-OMe modification can occur on any nucleotide of the sense strand or antisense strand. For instance, the 2′-OMe modification can occur on every nucleotide on the sense strand and/or antisense strand; each thermally stabilizing modification can occur in an alternating pattern on the sense strand or antisense strand; or the sense strand and antisense strand both comprise 2′-OMe modifications in an alternating pattern. The alternating pattern of the thermally stabilizing modifications on the sense strand can be the same or different from the antisense strand, and the alternating pattern of the thermally stabilizing modifications on the sense strand can have a shift relative to the alternating pattern of the 2′-OMe modifications on the antisense strand.

### Other Modified Nucleotides

In some embodiments, the oligonucleotide or dsRNA described herein can comprise locked nucleic acid (LNA). For example, the oligonucleotide or dsRNA described herein can comprise at least one, e.g., one, two, three, four, five, six, seven, eight, nine, ten, eleven, twelve, thirteen, fourteen, fifteen, sixteen, seventeen, eighteen, nineteen, twenty or more LNA modifications. It is noted that the LNA nucleotides all can be present in one strand of the dsRNA.

In some embodiments, both the sense and the antisense strands comprise at least LNA modifications. The LNA modification can occur on any nucleotide of the sense strand or antisense strand. For instance, the LNA modification can occur on every nucleotide on the sense strand and/or antisense strand; each LNA modification can occur in an alternating pattern on the sense strand or antisense strand; or the sense strand and antisense strand both comprise LNA modifications in an alternating pattern. The alternating pattern of the LNA modifications on the sense strand can be the same or different from the antisense strand, and the alternating pattern of the LNA modifications on the sense strand can have a shift relative to the alternating pattern of the 2′-fluoro modifications on the antisense strand.

The antisense strand can comprise at least one, e.g., two, three, four, five, six, seven, eight, nine, ten or more LNA modifications. Without limitations, a LNA modification in the antisense strand can be present at any position.

The sense strand can comprise at least one, e.g., two, three, four, five, six, seven, eight, nine, ten or more LNA modifications. Without limitations, a LNA modification in the sense strand can be present at any position. In some embodiments, the sense strand comprises at least one, e.g., two, three, four, five, six, seven, eight, nine, ten or more LNA modifications and the antisense strand does not comprise a 2′-fluoro nucleotide at positions 3-9, counting from 5′-end.

The oligonucleotide or dsRNA described herein can comprise bridged nucleic acid (BNA). For example, the oligonucleotide or dsRNA described herein can comprise at least one, e.g., one, two, three, four, five, six, seven, eight, nine, ten, eleven, twelve, thirteen, fourteen, fifteen, sixteen, seventeen, eighteen, nineteen, twenty or more BNA modifications. Without limitations, the BNA nucleotides all can be present in one of the dsRNA. In some embodiments, both the sense and the antisense strands comprise at least BNA modifications. The BNA modification can occur on any nucleotide of the sense strand or antisense strand. For instance, the BNA modification can occur on every nucleotide on the sense strand and/or antisense strand; each BNA modification can occur in an alternating pattern on the sense strand or antisense strand; or the sense strand and antisense strand both comprise BNA modifications in an alternating pattern. The alternating pattern of the BNA modifications on the sense strand can be the same or different from the antisense strand, and the alternating pattern of the BNA modifications on the sense strand can have a shift relative to the alternating pattern of the 2′-fluoro modifications on the antisense strand.

The antisense strand can comprise at least one, e.g., two, three, four, five, six, seven, eight, nine, ten or more BNA modifications. Without limitations, a BNA modification in the antisense strand can be present at any position.

The sense strand can comprise at least one, e.g., two, three, four, five, six, seven, eight, nine, ten or more BNA modifications. Without limitations, a BNA modification in the sense strand can be present at any position. In some embodiments, the sense strand comprises at least one, e.g., two, three, four, five, six, seven, eight, nine, ten or more BNA modifications and the antisense strand does not comprise a 2′-fluoro nucleotide at positions 3-9, counting from 5′-end.

The oligonucleotide or dsRNA described herein can comprise cyclohexene nucleic acid (CeNA). For example, the oligonucleotide or dsRNA described herein can comprise at least one, e.g., one, two, three, four, five, six, seven, eight, nine, ten, eleven, twelve, thirteen, fourteen, fifteen, sixteen, seventeen, eighteen, nineteen, twenty or more CeNA modifications. Without limitations, the CeNA nucleotides all can be present in one strand of the dsRNA. In some embodiments, both the sense and the antisense strands comprise at least CeNA modifications. The CeNA modification can occur on any nucleotide of the sense strand or antisense strand. For instance, the CeNA modification can occur on every nucleotide on the sense strand and/or antisense strand; each CeNA modification can occur in an alternating pattern on the sense strand or antisense strand; or the sense strand and antisense strand both comprise CeNA modifications in an alternating pattern. The alternating pattern of the CeNA modifications on the sense strand can be the same or different from the antisense strand, and the alternating pattern of the CeNA modifications on the sense strand can have a shift relative to the alternating pattern of the 2′-fluoro modifications on the antisense strand.

The antisense strand can comprise at least one, e.g., two, three, four, five, six, seven, eight, nine, ten or more CeNA modifications. Without limitations, a CeNA modification in the antisense strand can be present at any position.

The sense strand can comprise at least one, e.g., two, three, four, five, six, seven, eight, nine, ten or more CeNA modifications. Without limitations, a CeNA modification in the sense strand can be present at any position. In some embodiments, the sense strand comprises at least one, e.g., two, three, four, five, six, seven, eight, nine, ten or more CeNA modifications and the antisense strand does not comprise a 2′-fluoro nucleotide at positions 3-9, counting from 5′-end.

The oligonucleotide or dsRNA described herein can comprise thermally stabilizing modifications. For example, the oligonucleotide or dsRNA described herein can comprise at least four, e.g., five, six, seven, eight, nine, ten, eleven, twelve, thirteen, fourteen, fifteen or more thermally stabilizing modifications. The thermally stabilizing modifications all can be present in one strand of the dsRNA.

In some embodiments, both the sense and the antisense strands comprise at least one, e.g., two, three, four or more thermally stabilizing modifications. The thermally stabilizing modification can occur on any nucleotide of the sense strand or antisense strand. For instance, the thermally stabilizing modification can occur on every nucleotide on the sense strand and/or antisense strand; each thermally stabilizing modification can occur in an alternating pattern on the sense strand or antisense strand; or the sense strand and antisense strand both comprise thermally stabilizing modifications in an alternating pattern. The alternating pattern of the thermally stabilizing modifications on the sense strand can be the same or different from the antisense strand, and the alternating pattern of the thermally stabilizing modifications on the sense strand can have a shift relative to the alternating pattern of the thermally stabilizing modifications on the antisense strand.

The antisense strand can comprise at least one, e.g., two, three, four, five, six, seven, eight, nine, ten or more thermally stabilizing modifications. In some embodiments, the antisense strand comprises two, three, four, five or six thermally stabilizing modifications. Without limitations, a thermally stabilizing modification in the antisense strand can be present at any position. In some embodiments, the antisense strand comprises at least three thermally stabilizing modifications. For example, the antisense strand comprises thermally stabilizing modifications at least at positions 2, 14 and 16 from the 5′-end. In some other embodiments, the antisense comprises at least four thermally stabilizing modifications. For example, the antisense comprises thermally stabilizing modifications at least at positions 2, 6, 14 and 16 from the 5′-end. In some further embodiments, the antisense strand comprises at least five thermally stabilizing modifications. For example, the antisense strand comprises thermally stabilizing modifications at least at positions 2, 6, 9, 14 and 16 from the 5′-end. In still some further embodiments, the antisense strand comprises at least six thermally stabilizing modifications. For example, the antisense strand comprises thermally stabilizing modifications at least at positions 2, 6, 8, 9, 14 and 16 from the 5′-end.

The sense strand can comprise at least one, e.g., two, three, four, five, six, seven, eight, nine, ten or more thermally stabilizing modifications. In some embodiments, the sense strand comprises two, three, four, or five thermally stabilizing modifications. For example, the sense strand comprises three or four thermally stabilizing modifications. Without limitations, a thermally stabilizing modification in the sense strand can be present at any positions. In some embodiments, the sense strand comprises at least three thermally stabilizing modifications. For example, the sense comprises thermally stabilizing modification at least at positions 7, 10 and 11 from the 5′-end. In some other embodiments, the sense strand comprises at least four thermally stabilizing modifications. For example, the sense comprises thermally stabilizing modification at least at positions 7, 9, 10 and 11 from the 5′-end.

In some embodiments, the sense strand comprises thermally stabilizing modifications at positions opposite or complimentary to positions 11, 12 and 15 of the antisense strand, counting from the 5′-end of the antisense strand. In some other embodiments, the sense strand comprises thermally stabilizing modifications at positions opposite or complimentary to positions 11, 12, 13 and 15 of the antisense strand, counting from the 5′-end of the antisense strand. In some embodiments, the sense strand comprises a block of two, three or four thermally stabilizing modification.

In some embodiments, the sense strand comprises thermally stabilizing modifications at least at positions 7, 9, and 11 from the 5′-end, and the antisense strand comprises thermally stabilizing modifications at least at positions 2, 14 and 16 from the 5′-end. In some other embodiments, the sense strand comprises thermally stabilizing modifications at least at positions 7, 9, and 11 from the 5′-end, and the antisense strand comprises thermally stabilizing modifications at least at positions 2, 6, 9, 14 and 16 from the 5′-end. In yet some other embodiments, the sense strand comprises thermally stabilizing modifications at least at positions 7, 9, and 11 from the 5′-end, and the antisense strand comprises thermally stabilizing modifications at least at positions 2, 6, 8, 9, 14 and 16 from the 5′-end.

In some embodiments, the sense strand comprises thermally stabilizing modifications at least at positions 7, 9, 10, and 11 from the 5′-end, and the antisense strand comprises thermally stabilizing modifications at least at positions 2, 14 and 16 from the 5′-end. In some other embodiments, the sense strand comprises thermally stabilizing modifications at least at positions 7, 9, 10, and 11 from the 5′-end, and the antisense strand comprises thermally stabilizing modifications at least at positions 2, 6, 9, 14 and 16 from the 5′-end. In yet some other embodiments, the sense strand comprises thermally stabilizing modifications at least at positions 7, 9, 10, and 11 from the 5′-end, and the antisense strand comprises thermally stabilizing modifications at least at positions 2, 6, 8, 9, 14 and 16 from the 5′-end.

In some embodiments, the sense strand does not comprise a thermally stabilizing modification in a position opposite or complimentary to the thermally destabilizing modification of the duplex in the antisense strand.

Exemplary thermally stabilizing modifications include, but are not limited to, 2′-fluoro modifications and locked nucleic acid (LNA).

### Internucleoside Linkages

As used herein, “internucleoside linkage” refers to a covalent linkage between adjacent nucleosides. The two main classes of internucleoside linkages are defined by the presence or absence of a phosphorus atom. Representative phosphorus containing linkages include, but are not limited to, phosphodiesters (P═O), phosphotriesters, methylphosphonates, phosphoramidate, and phosphorothioates (P═S). Representative non-phosphorus containing linking groups include, but are not limited to, methylenemethylimino (—CH₂—N(CH₃)—O—CH₂—), thiodiester (—O—C(O)—S—), thionocarbamate (—O—C(O)(NH)—S—); siloxane (—O—Si(H)₂—O—); and N,N′-dimethylhydrazine (—CH₂—N(CH₃)—N(CH₃)—). Modified internucleoside linkages, compared to natural phosphodiester linkages, can be used to alter, typically increase, nuclease resistance of the oligonucleotide compound. In certain embodiments, linkages having a chiral atom can be prepared as racemic mixtures, as separate enantiomers. Representative chiral linkages include, but are not limited to, alkylphosphonates and phosphorothioates. Methods of preparation of phosphorous-containing and non-phosphorous-containing linkages are well known to those skilled in the art.

The phosphate group in the internucleoside linkage can be modified by replacing one of the oxygens with a different substituent. One result of this modification can be increased resistance of the oligonucleotide to nucleolytic breakdown. Examples of modified phosphate groups include phosphorothioate, phosphoroselenates, borano phosphates, borano phosphate esters, hydrogen phosphonates, phosphoroamidates, alkyl or aryl phosphonates and phosphotriesters. In some embodiments, one of the non-bridging phosphate oxygen atoms in the phosphodiester internucleoside linkage can be replaced by any of the following: S, Se, BR3 (R is hydrogen, alkyl, aryl), C (i.e. an alkyl group, an aryl group, etc.,), H, NR₂ (R is hydrogen, optionally substituted alkyl, aryl), or OR (R is optionally substituted alkyl or aryl). The phosphorous atom in an unmodified phosphate group is achiral. However, replacement of one of the non-bridging oxygens with one of the above atoms or groups of atoms renders the phosphorous atom chiral. In other words a phosphorous atom in a phosphate group modified in this way is a stereogenic center. The stereogenic phosphorous atom can possess either the “R” configuration (herein Rp) or the “S” configuration (herein Sp).

Phosphorodithioates have both non-bridging oxygens replaced by sulfur. The phosphorus center in the phosphorodithioates is achiral which precludes the formation of oligonucleotides diastereomers. Thus, while not wishing to be bound by theory, modifications to both non-bridging oxygens, which eliminate the chiral center, e.g. phosphorodithioate formation, can be desirable in that they cannot produce diastereomer mixtures. The non-bridging oxygens can be independently any one of O, S, Se, B, C, H, N, or OR (R is alkyl or aryl).

A phosphodiester internucleoside linkage can also be modified by replacement of bridging oxygen, (i.e. oxygen that links the phosphate to the sugar of the nucleosides), with nitrogen (bridged phosphoroamidates), sulfur (bridged phosphorothioates) and carbon (bridged methylenephosphonates). The replacement can occur at the either one of the linking oxygens or at both linking oxygens. When the bridging oxygen is the 3′-oxygen of a nucleoside, replacement with carbon is preferred. When the bridging oxygen is the 5′-oxygen of a nucleoside, replacement with nitrogen is preferred.

Modified phosphate linkages where at least one of the oxygen linked to the phosphate has been replaced or the phosphate group has been replaced by a non-phosphorous group, are also referred to as “non-phosphodiester intersugar linkage” or “non-phosphodiester linker.”

In certain embodiments, the phosphate group can be replaced by non-phosphorus containing connectors, e.g. dephospho linkers. Dephospho linkers are also referred to as non-phosphodiester linkers herein. While not wishing to be bound by theory, it is believed that since the charged phosphodiester group is the reaction center in nucleolytic degradation, its replacement with neutral structural mimics should impart enhanced nuclease stability. Again, while not wishing to be bound by theory, it can be desirable, in some embodiment, to introduce alterations in which the charged phosphate group is replaced by a neutral moiety.

Examples of moieties which can replace the phosphate group include, but are not limited to, amides (for example amide-3 (3′-CH₂—C(═O)—N(H)-5′) and amide-4 (3′-CH₂—N(H)—C(═O)-5′)), hydroxylamino, siloxane (dialkylsiloxane), carboxamide, carbonate, carboxymethyl, carbamate, carboxylate ester, thioether, ethylene oxide linker, sulfide, sulfonate, sulfonamide, sulfonate ester, thioformacetal (3′-S—CH₂—O-5′), formacetal (3 ‘—O—CH₂—O-5’), oxime, methyleneimino, methykenecarbonylamino, methylenemethylimino (MMI, 3′-CH₂—N(CH₃)—O-5′), methylenehydrazo, methylenedimethylhydrazo, methyleneoxymethylimino, ethers (C3′—O—C5′), thioethers (C3′—S—C5′), thioacetamido (C3′—N(H)—C(═O)—CH₂—S—C5′, C3′—O—P(O)—O—SS—C5′, C3′—CH₂—NH—NH—C5′, 3′—NHP(O)(OCH₃)—O-5′ and 3′-NHP(O)(OCH₃)—O-5′ and nonionic linkages containing mixed N, O, S and CH₂ component parts. See for example, Carbohydrate Modifications in Antisense Research; Y. S. Sanghvi and P.D. Cook Eds. ACS Symposium Series 580; Chapters 3 and 4, (pp. 40-65). Preferred embodiments include methylenemethylimino (MMI), methylenecarbonylamino, amides, carbamate and ethylene oxide linker.

One skilled in the art is well aware that in certain instances replacement of a non-bridging oxygen can lead to enhanced cleavage of the intersugar linkage by the neighboring 2′-OH, thus in many instances, a modification of a non-bridging oxygen can necessitate modification of 2′-OH, e.g., a modification that does not participate in cleavage of the neighboring intersugar linkage, e.g., arabinose sugar, 2′-O-alkyl, 2′-F, LNA and ENA.

Preferred non-phosphodiester internucleoside linkages include phosphorothioates, phosphorothioates with an at least 1%, 5%, 10%, 20%, 30%, 40%, 50%, 60%, 70%, 80%, 90% 95% or more enantiomeric excess of Sp isomer, phosphorothioates with an at least 1%, 5%, 10%, 20%, 30%, 40%, 50%, 60%, 70%, 80%, 90% 95% or more enantiomeric excess of Rp isomer, phosphorodithioates, phsophotriesters, aminoalkylphosphotrioesters, alkyl-phosphonaters (e.g., methyl-phosphonate), selenophosphates, phosphoramidates (e.g., N-alkylphosphoramidate), and boranophosphonates.

Additional exemplary non-phosphorus containing internucleoside linking groups are described in U.S. Pat. Nos. 5,034,506; 5,166,315; 5,185,444; 5,214,134; 5,216,141; 5,235,033; 5,264,562; 5,264,564; 5,405,938; 5,434,257; 5,466,677; 5,470,967; 5,489,677; 5,541,307; 5,561,225; 5,596,086; 5,602,240; 5,610,289; 5,602,240; 5,608,046; 5,610,289; 5,618,704; 5,623,070; 5,663,312; 5,633,360; 5,677,437; 5,792,608; 5,646,269 and 5,677,439, content of each of which is incorporated herein by reference.

In some embodiments of any one of the aspects, the antisense and/or the sense strand comprises one or more neutral internucleoside linkages that are non-ionic. Suitable neutral internucleoside linkages include, but are not limited to, phosphotriesters, methylphosphonates, MMI (3′-CH₂—N(CH₃)—O-5′), amide-3 (3′-CH₂—C(═O)—N(H)-5′), amide-4 (3′-CH₂—N(H)—C(═O)-5′), formacetal (3 ‘—O—CH₂—O-5’), and thioformacetal (3′-S—CH₂—O-5′); nonionic linkages containing siloxane (dialkylsiloxane), carboxylate ester, carboxamide, sulfide, sulfonate ester and/or amides (See for example: Carbohydrate Modifications in Antisense Research; Y. S. Sanghvi and P.D. Cook Eds. ACS Symposium Series 580; Chapters 3 and 4, (pp. 40-65)); and nonionic linkages containing mixed N, O, S and CH₂ component parts.

In some embodiments, the non-phosphodiester backbone linkage is selected from the group consisting of phosphorothioate, phosphorodithioate, alkyl-phosphonate and phosphoramidate backbone linkages.

In some embodiments of any one of the aspects described herein, the internucleoside

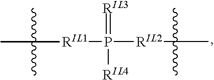

linkage is where R^{IL1} and R^{IL2} are each independently for each occurrence absent, O, S, CH₂, NR (R is hydrogen, alkyl, aryl), or optionally substituted alkylene, wherein backbone of the alkylene can comprise one or more of O, S, SS and NR (R is hydrogen, alkyl, aryl) internally and/or at the end; and R^{IL3} and R^{IL4} are each independently selected from the group consisting of O, OR (R is hydrogen, alkyl, aryl), S, Se, BR3 (R is hydrogen, alkyl, aryl), BH3, C (i.e. an alkyl group, an aryl group, etc.,), H, NR₂ (R is hydrogen, alkyl, aryl), alkyl or aryl. It is understood that one of R^{IL1} and R^{IL2} is replacing the oxygen linked to 5′ carbon of a first nucleoside sugar and the other of R^{IL1} and R^{IL2} is replacing the oxygen linked to 3′ (or 2′) carbon of a second nucleoside sugar.

In some embodiments of any one of the aspects, R^{IL1}, R^{IL2}, R^{IL3} and R^{IL4} all are O.

In some embodiments, R^{IL1} and R^{IL2} are O and at least one of R^{IL3} and R^{IL4} is other than O. For example, one of R^{IL3} and R^{IL4} is S and the other is O or both of R^{IL3} and R^{IL4} are S.

In some embodiments of any one of the aspects described herein, R²³ is a bond to a modified internucleoside linkage, e.g., an internucleoside linkage of structure:

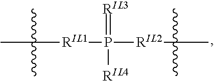

where at least one of RIL1, R^{IL2}, R^{IL3} and R^{IL4} is not O. For example, at least one of R^{IL3} and R^{IL4} is S.

In some embodiments of any one of the aspects described herein, R²³ or R²² is a bond to a phosphorothioate internucleoside linkage, e.g., an internucleoside linkage of structure:

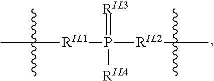

where at least one of R^{IL1} and R^{IL2} are O; one of R^{IL3} and R^{IL4} is O and the other of R^{IL3} and R^{IL4} is S.

In some embodiments of any one of the aspects described herein, R²³ or R²² is a bond to phosphodiester internucleoside linkage, e.g., an internucleoside linkage of structure:

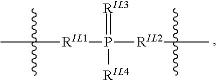

where R^{IL1}, R^{IL2}, R^{IL3} and R^{IL4} are O.

In some embodiments of any one of the aspects, the antisense and/or the sense strand can comprise one or more, e.g., 1, 2, 3, 4, 5, 6, 7, 8 or more modified internucleoside linkages. For example, the antisense and/or the sense strand can comprise 1, 2, 3, 4, 5 or 6 modified internucleoside linkages. For example, the antisense and/or the sense strand comprises 1, 2, 3 or 4 modified internucleoside linkages. In some embodiments, the antisense and/or the sense strand comprises at least two modified internucleoside linkages between the first five nucleotides counting from the 5′-end of the strand and further comprises at least two modified internucleoside linkages between the first five nucleotides counting from the 3′-end of the strand. For example, the antisense and/or the sense strand comprises modified internucleoside linkages between nucleotides 1 and 2, and between nucleotides 2 and 3, counting from 5′-end of the strand, and between nucleotides 1 and 2, and between nucleotides 2 and 3, counting from 3′-end of the strand.

In some embodiments of any one of the aspects, the modified internucleoside linkage is a phosphorothioate. Accordingly, in some embodiments of any one of the aspects, the antisense and/or the sense strand comprises one or more, e.g., 1, 2, 3, 4, 5, 6, 7, 8 or more phosphorothioate internucleoside linkages. For example, the antisense and/or the sense strand comprises 1, 2, 3, 4, 5 or 6 phosphorothioate internucleoside linkages. For example, the antisense and/or the sense strand comprises 1, 2, 3 or 4 phosphorothioate internucleoside linkages. In some embodiments, the antisense and/or the sense strand comprises at least two phosphorothioate internucleoside linkages between the first five nucleotides counting from the 5′-end of the strand and further comprises at least two phosphorothioate internucleoside linkages between the first five nucleotides counting from the 3′-end of the strand. For example, the antisense and/or the sense strand comprises modified internucleoside linkages between nucleotides 1 and 2, and between nucleotides 2 and 3, counting from 5′-end of the strand, and between nucleotides 1 and 2, and between nucleotides 2 and 3, counting from 3′-end of the strand.

### Phosphorothioates

The oligonucleotide or dsRNA described herein can comprise at least one, e.g., two, three, four, five, six, seven, eight, nine, ten or more phosphorothioate or methylphosphonate internucleotide linkage. The phosphorothioate or methylphosphonate internucleotide linkage modification can occur on any nucleotide of the oligonucleotide or dsRNA described herein.

In the dsRNA, the phosphorothioate or methylphosphonate internucleotide linkage modification can occur in the sense strand or antisense strand or both in any position of the strand. For instance, the internucleotide linkage modification can occur on every nucleotide on the sense strand and/or antisense strand; each internucleotide linkage modification can occur in an alternating pattern on the sense strand or antisense strand; or the sense strand or antisense strand comprises both internucleotide linkage modifications in an alternating pattern. The alternating pattern of the internucleotide linkage modification on the sense strand can be the same or different from the antisense strand, and the alternating pattern of the internucleotide linkage modification on the sense strand can have a shift relative to the alternating pattern of the internucleotide linkage modification on the antisense strand.

In some embodiments, the dsRNA comprises the phosphorothioate or methylphosphonate internucleotide linkage modification in the overhang region. For example, the overhang region comprises two nucleotides having a phosphorothioate or methylphosphonate internucleotide linkage between the two nucleotides. Internucleotide linkage modifications also may be made to link the overhang nucleotides with the terminal paired nucleotides within duplex region. For example, at least 2, 3, 4, or all the overhang nucleotides can be linked through phosphorothioate or methylphosphonate internucleotide linkage, and optionally, there may be additional phosphorothioate or methylphosphonate internucleotide linkages linking the overhang nucleotide with a paired nucleotide that is next to the overhang nucleotide. For instance, there may be at least two phosphorothioate internucleotide linkages between the terminal three nucleotides, in which two of the three nucleotides are overhang nucleotides, and the third is a paired nucleotide next to the overhang nucleotide. Preferably, these terminal three nucleotides can be at the 3′-end of the antisense strand.

In some embodiments, the sense strand comprises 1-10 blocks of two to ten phosphorothioate or methylphosphonate internucleotide linkages separated by 1, 2, 3, 4, 5, 6, 7, 8, 9, 10, 11, 12, 13, 14, 15 or 16 phosphate internucleotide linkages, wherein one of the phosphorothioate or methylphosphonate internucleotide linkages is placed at any position in the sense strand and the said sense strand is paired with an antisense strand comprising any combination of phosphorothioate, methylphosphonate and phosphate internucleotide linkages or an antisense strand comprising either phosphorothioate or methylphosphonate or phosphate linkage.

In some embodiments, the antisense strand comprises two blocks of two phosphorothioate or methylphosphonate internucleotide linkages separated by 1, 2, 3, 4, 5, 6, 7, 8, 9, 10, 11, 12, 13, 14, 15, 16, 17, or 18 phosphate internucleotide linkages, wherein one of the phosphorothioate or methylphosphonate internucleotide linkages is placed at any position in the antisense strand and the said antisense strand is paired with a sense strand comprising any combination of phosphorothioate, methylphosphonate and phosphate internucleotide linkages or an antisense strand comprising either phosphorothioate or methylphosphonate or phosphate linkage.

In some embodiments, the antisense strand comprises two blocks of three phosphorothioate or methylphosphonate internucleotide linkages separated by 1, 2, 3, 4, 5, 6, 7, 8, 9, 10, 11, 12, 13, 14, 15 or 16 phosphate internucleotide linkages, wherein one of the phosphorothioate or methylphosphonate internucleotide linkages is placed at any position in antisense strand and the said antisense strand is paired with a sense strand comprising any combination of phosphorothioate, methylphosphonate and phosphate internucleotide linkages or an antisense strand comprising either phosphorothioate or methylphosphonate or phosphate linkage.

In some embodiments, the antisense strand comprises two blocks of four phosphorothioate or methylphosphonate internucleotide linkages separated by 1, 2, 3, 4, 5, 6, 7, 8, 9, 10, 11, 12, 13 or 14 phosphate internucleotide linkages, wherein one of the phosphorothioate or methylphosphonate internucleotide linkages is placed at any position in the antisense strand and the said antisense strand is paired with a sense strand comprising any combination of phosphorothioate, methylphosphonate and phosphate internucleotide linkages or an antisense strand comprising either phosphorothioate or methylphosphonate or phosphate linkage.

In some embodiments, the antisense strand comprises two blocks of five phosphorothioate or methylphosphonate internucleotide linkages separated by 1, 2, 3, 4, 5, 6, 7, 8, 9, 10, 11 or 12 phosphate internucleotide linkages, wherein one of the phosphorothioate or methylphosphonate internucleotide linkages is placed at any position in the antisense strand and the said antisense strand is paired with a sense strand comprising any combination of phosphorothioate, methylphosphonate and phosphate internucleotide linkages or an antisense strand comprising either phosphorothioate or methylphosphonate or phosphate linkage.

In some embodiments, the antisense strand comprises two blocks of six phosphorothioate or methylphosphonate internucleotide linkages separated by 1, 2, 3, 4, 5, 6, 7, 8, 9 or 10 phosphate internucleotide linkages, wherein one of the phosphorothioate or methylphosphonate internucleotide linkages is placed at any position in the antisense strand and the said antisense strand is paired with a sense strand comprising any combination of phosphorothioate, methylphosphonate and phosphate internucleotide linkages or an antisense strand comprising either phosphorothioate or methylphosphonate or phosphate linkage.

In some embodiments, the antisense strand comprises two blocks of seven phosphorothioate or methylphosphonate internucleotide linkages separated by 1, 2, 3, 4, 5, 6, 7 or 8 phosphate internucleotide linkages, wherein one of the phosphorothioate or methylphosphonate internucleotide linkages is placed at any position in the antisense strand and the said antisense strand is paired with a sense strand comprising any combination of phosphorothioate, methylphosphonate and phosphate internucleotide linkages or an antisense strand comprising either phosphorothioate or methylphosphonate or phosphate linkage.

In some embodiments, the antisense strand comprises two blocks of eight phosphorothioate or methylphosphonate internucleotide linkages separated by 1, 2, 3, 4, 5 or 6 phosphate internucleotide linkages, wherein one of the phosphorothioate or methylphosphonate internucleotide linkages is placed at any position in the antisense strand and the said antisense strand is paired with a sense strand comprising any combination of phosphorothioate, methylphosphonate and phosphate internucleotide linkages or an antisense strand comprising either phosphorothioate or methylphosphonate or phosphate linkage.

In some embodiments, the antisense strand comprises two blocks of nine phosphorothioate or methylphosphonate internucleotide linkages separated by 1, 2, 3 or 4 phosphate internucleotide linkages, wherein one of the phosphorothioate or methylphosphonate internucleotide linkages is placed at any position in antisense strand sequence and the said antisense strand is paired with a sense strand comprising any combination of phosphorothioate, methylphosphonate and phosphate internucleotide linkages or an antisense strand comprising either phosphorothioate or methylphosphonate or phosphate linkage.

In some embodiments, the dsRNA comprises one or more phosphorothioate or methylphosphonate internucleotide linkage modification within 1-10 of the termini position(s) of the sense and/or antisense strand. For example, at least 2, 3, 4, 5, 6, 7, 8, 9 or 10 nucleotides may be linked through phosphorothioate or methylphosphonate internucleotide linkage at one end or both ends of the sense and/or antisense strand.

In some embodiments, the dsRNA comprises one or more phosphorothioate or methylphosphonate internucleotide linkage modification within 1-10 of the internal region of the duplex of each of the sense and/or antisense strand. For example, at least 2, 3, 4, 5, 6, 7, 8, 9 or 10 nucleotides may be linked through phosphorothioate methylphosphonate internucleotide linkage at position 8-16 of the duplex region counting from the 5′-end of the sense strand; dsRNA can optionally further comprise one or more phosphorothioate or methylphosphonate internucleotide linkage modification within 1-10 of the termini position(s).

In some embodiments, the dsRNA comprises one to five phosphorothioate or methylphosphonate internucleotide linkage modification(s) within position 1-5 (counting from the 5′-end) and one to five phosphorothioate or methylphosphonate internucleotide linkage modification(s) within position 1-5 (counting from the 3′-end) of the sense strand, and one to five phosphorothioate or methylphosphonate internucleotide linkage modification at positions 1 and 2 (counting from the 5′-end) and one to five within positions 1-5 (counting from the 3′-end) of the antisense strand.

In some embodiments, the dsRNA comprises one phosphorothioate internucleotide linkage modification within position 1-5 (counting from the 5′-end) and one phosphorothioate or methylphosphonate internucleotide linkage modification within position 1-5 (counting from the 3′-end) of the sense strand, and one phosphorothioate internucleotide linkage modification at positions 1 and 2 (counting from the 5′-end) and two phosphorothioate or methylphosphonate internucleotide linkage modifications within positions 1-5 (counting from the 3′-end) of the antisense strand.

In some embodiments, the dsRNA comprises two phosphorothioate internucleotide linkage modifications within position 1-5 (counting from the 5′-end) and one phosphorothioate internucleotide linkage modification within position 1-5 (counting from the 3′-end) of the sense strand, and one phosphorothioate internucleotide linkage modification at positions 1 and 2 (counting from the 5′-end) and two phosphorothioate internucleotide linkage modifications within positions 18-23 (counting from the 3′-end) of the antisense strand.

In some embodiments, the dsRNA comprises two phosphorothioate internucleotide linkage modifications within position 1-5 (counting from the 5′-end) and two phosphorothioate internucleotide linkage modifications within position 1-5 (counting from the 3′-end) of the sense strand, and one phosphorothioate internucleotide linkage modification at positions 1 and 2 (counting from the 5′-end) and two phosphorothioate internucleotide linkage modifications within positions 1-5 (counting from the 3′-end) of the antisense strand.

In some embodiments, the dsRNA comprises two phosphorothioate internucleotide linkage modifications within position 1-5 (counting from the 5′-end) and two phosphorothioate internucleotide linkage modifications within position 1-5 (counting from the 3′-end) of the sense strand, and one phosphorothioate internucleotide linkage modification at positions 1 and 2 (counting from the 5′-end) and one phosphorothioate internucleotide linkage modification within positions 1-5 (counting from the 3′-end) of the antisense strand.

In some embodiments, the dsRNA comprises one phosphorothioate internucleotide linkage modification within position 1-5 (counting from the 5′-end) and one phosphorothioate internucleotide linkage modification within position 1-5 (counting from the 3′-end) of the sense strand, and two phosphorothioate internucleotide linkage modifications at positions 1 and 2 (counting from the 5′-end) and two phosphorothioate internucleotide linkage modifications within positions 1-5 (counting from the 3′-end) of the antisense strand.

In some embodiments, the dsRNA comprises one phosphorothioate internucleotide linkage modification within position 1-5 (counting from the 5′-end) and one within position 1-5 (counting from the 3′-end) of the sense strand, and two phosphorothioate internucleotide linkage modification at positions 1 and 2 (counting from the 5′-end) and one phosphorothioate internucleotide linkage modification within positions 1-5 (counting from the 3′-end) of the antisense strand.

In some embodiments, the dsRNA comprises one phosphorothioate internucleotide linkage modification within position 1-5 (counting from the 5′-end) of the sense strand, and two phosphorothioate internucleotide linkage modifications at positions 1 and 2 (counting from the 5′-end) and one phosphorothioate internucleotide linkage modification within positions 1-5 (counting from the 3′-end) of the antisense strand.

In some embodiments, the dsRNA comprises two phosphorothioate internucleotide linkage modifications within position 1-5 (counting from the 5′-end) of the sense strand, and one phosphorothioate internucleotide linkage modification at positions 1 and 2 (counting from the 5′-end) and two phosphorothioate internucleotide linkage modifications within positions 1-5 (counting from the 3′-end) of the antisense strand.

In some embodiments, the dsRNA comprises two phosphorothioate internucleotide linkage modifications within position 1-5 (counting from the 5′-end) and one within position 1-5 (counting from the 3′-end) of the sense strand, and two phosphorothioate internucleotide linkage modifications at positions 1 and 2 (counting from the 5′-end) and one phosphorothioate internucleotide linkage modification within positions 1-5 (counting from the 3′-end) of the antisense strand.

In some embodiments, the dsRNA comprises two phosphorothioate internucleotide linkage modifications within position 1-5 (counting from the 5′-end) and one phosphorothioate internucleotide linkage modification within position 1-5 (counting from the 3′-end) of the sense strand, and two phosphorothioate internucleotide linkage modifications at positions 1 and 2 (counting from the 5′-end) and two phosphorothioate internucleotide linkage modifications within positions 1-5 (counting from the 3′-end) of the antisense strand.

In some embodiments, the dsRNA comprises two phosphorothioate internucleotide linkage modifications within position 1-5 (counting from the 5′-end) and one phosphorothioate internucleotide linkage modification within position 1-5 (counting from the 3′-end) of the sense strand, and one phosphorothioate internucleotide linkage modification at positions 1 and 2 (counting from the 5′-end) and two phosphorothioate internucleotide linkage modifications within positions 1-5 (counting from the 3′-end) of the antisense strand.

In some embodiments, the dsRNA comprises two phosphorothioate internucleotide linkage modifications at position 1 and 2 (counting from the 5′-end), and two phosphorothioate internucleotide linkage modifications at position 1 and 2 (counting from the 3′-end) of the sense strand (counting from the 5′-end), and one phosphorothioate internucleotide linkage modification at positions 1 (counting from the 5′-end) and one at position 1 or 2 (counting from the 3′-end) of the antisense strand.

In some embodiments, the dsRNA comprises one phosphorothioate internucleotide linkage modification at position 1 (counting from the 5′-end), and one phosphorothioate internucleotide linkage modification at position 1 (counting from the 3′-end) of the sense strand, and two phosphorothioate internucleotide linkage modifications at positions 1 and 2 (counting from the 5′-end) and two phosphorothioate internucleotide linkage modifications at positions 1 and 2 (counting from the 3′-end) the antisense strand.

In some embodiments, the dsRNA comprises two phosphorothioate internucleotide linkage modifications at position 1 and 2 (counting from the 5′-end), and two phosphorothioate internucleotide linkage modifications at position 1 and 2 (counting from the 3′-end) of the sense strand, and one phosphorothioate internucleotide linkage modification at positions 1 (counting from the 5′-end) and one phosphorothioate internucleotide linkage modification at position 1 (counting from the 3′-end) of the antisense strand.

In some embodiments, the dsRNA comprises one phosphorothioate internucleotide linkage modification at position 1 (counting from the 5′-end), and one phosphorothioate internucleotide linkage modification at position 1 (counting from the 3′-end) of the sense strand, and two phosphorothioate internucleotide linkage modifications at positions 1 and 2 (counting from the 5′-end) and two phosphorothioate internucleotide linkage modifications at positions 1 and 2 (counting from the 5′-end) the antisense strand.

In some embodiments, the dsRNA comprises two phosphorothioate internucleotide linkage modifications at position 1 and 2 (counting from the 5′-end), and two phosphorothioate internucleotide linkage modifications at position 1 and 2 (counting from the 3′-end) of the sense strand, and one phosphorothioate internucleotide linkage modification at positions 1 (counting from the 5′-end) and one phosphorothioate internucleotide linkage modification at position 1 (counting from the 3′-end) of the antisense strand.

In some embodiments, the dsRNA one phosphorothioate internucleotide linkage modification at position 1 (counting from the 5′-end), and one phosphorothioate internucleotide linkage modification at position 1 (counting from the 3′-end) of the sense strand, and two phosphorothioate internucleotide linkage modifications at positions 1 and 2 (counting from the 5′-end) and two phosphorothioate internucleotide linkage modifications at positions 1 and 2 (counting from the 3′-end) of the antisense strand.

In some embodiments, the sense strand can comprise 0, 1, 2, 3 or 4 phosphorothioate internucleotide linkages. For example, the sense strand comprises phosphorothioate internucleotide linkages between nucleotide positions 1 and 2, and between nucleotide positions 2 and 3 (counting from the 5′-end).

In some embodiments, the antisense strand can comprise 1, 2, 3 or 4 phosphorothioate internucleotide linkages. For example, the sense strand comprises phosphorothioate internucleotide linkages between nucleotide positions 1 and 2, and between nucleotide positions 2 and 3 (counting from the 3′-end). In an additional example, the antisense strand comprises phosphorothioate internucleotide linkages between nucleotide positions 1 and 2 (counting from the 5′-end), between nucleotide positions 2 and 3 (counting from the 5′-end), between nucleotide positions 1 and 2 (counting from the 3′-end), and between nucleotide positions 2 and 3 (counting from the 3′-end).

In some embodiments, the sense strand comprises phosphorothioate internucleotide linkages between nucleotide positions 1 and 2 (counting from the 5′-end), and between nucleotide positions 2 and 3 (counting from the 5′-end), and the antisense strand comprises phosphorothioate internucleotide linkages between nucleotide positions 1 and 2 (counting from the 3′-end), and between nucleotide positions 2 and 3 (counting from the 5′-end). For example, the sense strand comprises phosphorothioate internucleotide linkages between nucleotide positions 1 and 2 (counting from the 5′-end), and between nucleotide positions 2 and 3 (counting from the 5′-end), and the antisense strand comprises phosphorothioate internucleotide linkages between nucleotide positions 1 and 2 (counting from the 5′-end), between nucleotide positions 2 and 3 (counting from the 5′-end), between nucleotide positions 1 and 2 (counting from the 3′-end), and between nucleotide positions 2 and 3 (counting from the 5′-end).

### 5′-Modifications

In some embodiments, the dsRNA can be 5′ phosphorylated or include a phosphoryl analog at 5′ terminus of the antisense and/or sense strand. For example, the antisense strand can be 5′ phosphorylated or include a phosphoryl analog at the 5′ terminus. Exemplary 5′-phosphate modifications include those which are compatible with RISC mediated gene silencing. Suitable modifications include: 5′-monophosphate ((HO)₂(O) P—O-5′); 5′-diphosphate ((HO)₂(O) P—O—P(HO)(O)—O-5′); 5′-triphosphate ((HO)₂(O) P—O—(HO)(O) P—O—P(HO)(O)—O-5′); 5′-guanosine cap (7-methylated or non-methylated) (7m-G-O-5′-(HO)(O) P—O—(HO)(O) P—O—P(HO)(O)—O-5′); 5′-adenosine cap (Appp), and any modified or unmodified nucleotide cap structure (N—O-5′-(HO)(O) P—O—(HO)(O) P—O—P(HO)(O)—O-5′); 5′-monothiophosphate (phosphorothioate; (HO) 2 (S) P—O-5′); 5′-monodithiophosphate (phosphorodithioate; (HO)(HS)(S) P—O-5′), 5′-phosphorothiolate ((HO)₂(O) P—S-5′); any additional combination of oxygen/sulfur replaced monophosphate, diphosphate and triphosphates (e.g. 5′-alpha-thiotriphosphate, 5′-gamma-thiotriphosphate, etc.), 5′-phosphoramidates ((HO)₂(O) P—NH-5′, (HO)(NH₂)(O) P—O-5′), 5′-alkylphosphonates (R=alkyl-methyl, ethyl, isopropyl, propyl, etc., e.g. R^{P} (OH)(O)—O-5′-, 5′-alkenylphosphonates (i.e. vinyl, substituted vinyl), (OH)₂(O) P-5′-CH₂—), 5′-alkyletherphosphonates (R=alkylether-methoxymethyl (MeOCH₂—), ethoxymethyl, etc., e.g. RP(OH)(O)—O-5′-).

In some embodiments, the antisense strand comprises a 5′-vinylphosphonate nucleotide at 5′-end. For example, the antisense strand comprises a 5′-E-vinylphosphanate nucleotide at 5′-end. In some embodiments, the antisense strand comprises 5′-E-vinylphosphanate and a nucleoside at position N-1 that reduces or inhibits activity of siRNA relative to a siRNA having the same antisense strand sequence, but unmodified N-1 position.

In some embodiments, the sense strand comprises a 5′-morpholino, a 5′-dimethylamino, a 5′-deoxy, an inverted abasic, or an inverted abasic locked nucleic acid modification at the 5′-end. In some embodiments, the sense strand comprises a nucleotide of Formula (II) at its 5′-end.

In some embodiments, the antisense strand comprises a nucleotide of Formula (II-VP) or (II-VP′) on its 5′-end.

### Thermal Stability

Generally, the dsRNA has a melting temperature in the range from about 40° C. to about 80° C. For example, the dsRNA has a melting temperature with a lower end of the range from about 40° C., 45° C., 50° C., 55° C., 60° C. or 65° C., and upper end of the range from about 70° C., 75° C. or 80° C. In some embodiments, the dsRNA has a melting temperature in the range from about 55° C. to about 70° C. or in the range from about 60° C. to about 75° C. In some embodiments, the dsRNA has a melting temperature in the range from about 57° C. to about 67° C. In some particular embodiments, the dsRNA has a melting temperature in the range from about 60° C. to about 67° C. In some additional embodiments, the dsRNA has a melting temperature in the range from about 62° C. to about 66° C.

Without wishing to be bound by a theory, thermally destabilizing modifications in the seed region of the antisense strand (i.e., at positions 2-9 from the 5′-end or positions 23-30 from the 3′-end of the antisense strand) can reduce or inhibit off-target gene silencing. Accordingly, the oligonucleotide or the dsRNA described herein can comprise at least one (e.g., one, two, three, four, five or more) thermally destabilizing modifications. In some embodiments, the antisense strand comprises at least one (e.g., one, two, three, four, five or more) thermally destabilizing modification of the duplex within the first 9 nucleotide positions of the 5′-end or nucleotide positions 23-31 from of the 3′-end of the antisense strand.

The term “thermally destabilizing modification(s)” includes modification(s) that would result with a dsRNA with a lower overall melting temperature (Tm) (preferably a Tm with one, two, three or four degrees lower than the Tm of the dsRNA without having such modification(s).

In some embodiments, thermally destabilizing modification is located at position 2, 3, 4, 5, 6, 7, 8 or 9, or preferably at position 4, 5, 6, 7, or 8, from the 5′-end of the antisense strand. In some embodiments, the thermally destabilizing modification is located at position 2, 3, 4, 5 or 9 from the 5′-end of the antisense strand. In some other embodiments, the thermally destabilizing modification is located at position 6, 7 or 8 from the 5′-end of the antisense strand. In some particular embodiments, the thermally destabilizing modification is located at position 7 from the 5′-end of the antisense strand.

The thermally destabilizing modifications can include, but are not limited to, abasic nucleosides; mismatch with the opposing nucleotide in the opposing strand; and nucleosides with modified sugars, such as 2′-deoxy nucleosides or acyclic nucleosides, e.g., unlocked nucleic acids (UNA) or glycol nucleic acid (GNA).

Exemplary abasic modifications include, but are not limited to, the following:

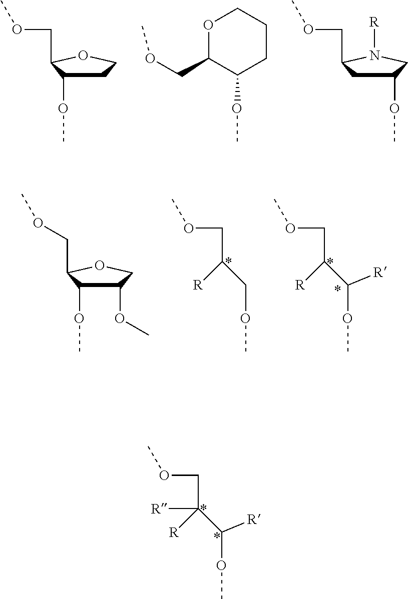

wherein R is H, Me, Et or OMe; R′ is H, Me, Et or OMe; R″ is H, Me, Et or OMe; and * represents either R, S or racemic.

Exemplary destabilizing sugar modifications include, but are not limited to the following:

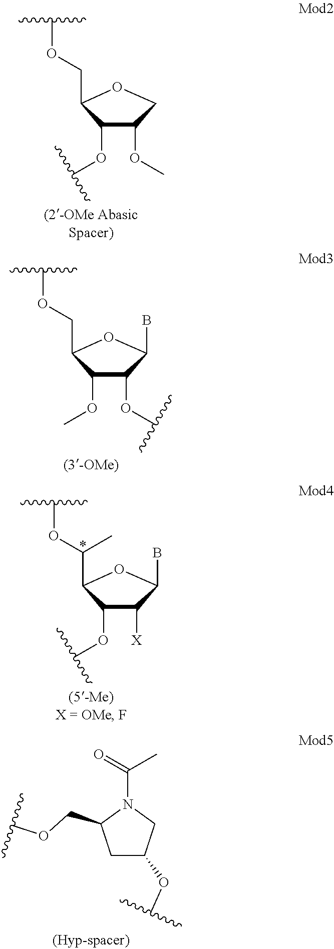

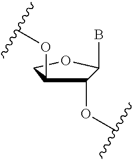

(TNA)

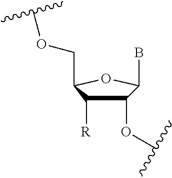

e.g., R = OH, F, OMe
2′-5′-linked (3′RNA)

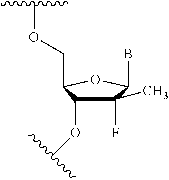

Me/F Gem

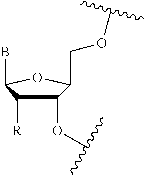

e.g., R = OH, F, OMe
L-nucleotide

wherein B is a modified or unmodified nucleobase.

Additional sugar modifications include, but are not limited to the following:

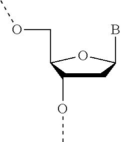

2′-deoxy

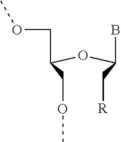

unlocked nucleic acid
R = H, OH, O-alkyl

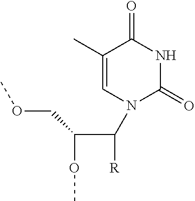

glycol nucleic acid
R = H, OH, O-alkyl

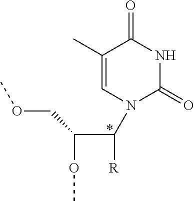

glycol nucleic acid
R = H, OH, O-alkyl

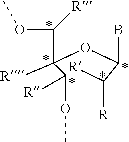

unlocked nucleic acid
R = H, OH, CH₃, CH₂CH₃, O-alkyl,
NH₂, NHMe, NMe₂
R′ = H, OH, CH₃, CH₂CH₃, O-alkyl,
NH₂, NHMe, NMe₂
R″ = H, OH, CH₃, CH₂CH₃, O-alkyl,
NH₂, NHMe, NMe₂
R′′′ = H, OH, CH₃, CH₂CH₃, O-alkyl,
NH₂, NHMe, NMe₂
R″″ = H, OH, CH₃, CH₂CH₃, O-alkyl,
NH₂, NHMe, NMe₂

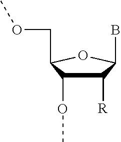

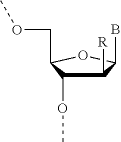

R = H, methyl, ethyl

wherein B is a modified or unmodified nucleobase.

In some embodiments the thermally destabilizing modification is selected from the group consisting of:

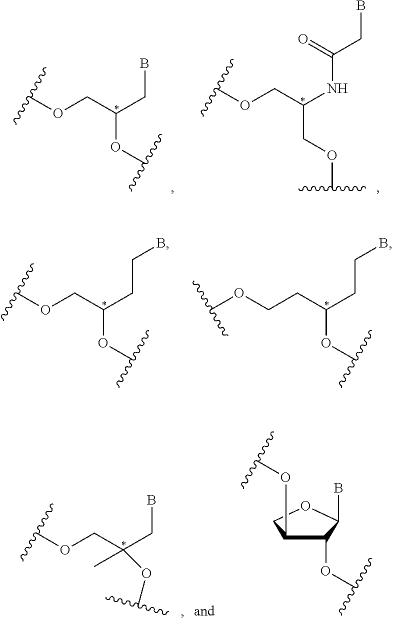

wherein B is a modified or unmodified nucleobase and the asterisk on each structure represents either R, S or racemic.

The term “acyclic nucleotide” refers to any nucleotide having an acyclic ribose sugar, for example, where any of bonds between the ribose carbons (e.g., C1′-C2′, C2′-C3′, C3′-C4′, C4′-O4′, or C1′-O4′) is absent and/or at least one of ribose carbons or oxygen (e.g., C1′, C2′, C3′, C4′ or O4′) are independently or in combination absent from the nucleotide. In some

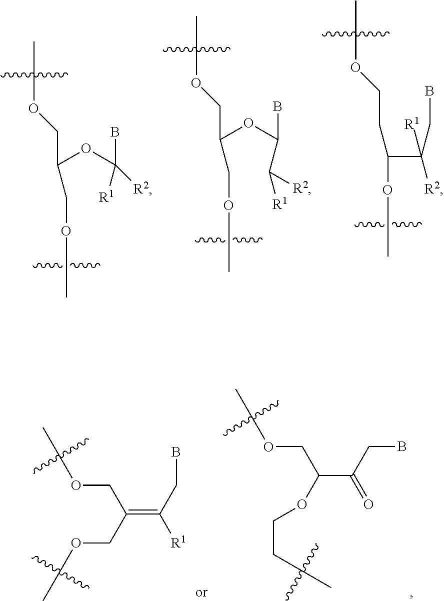

embodiments, acyclic nucleotide is wherein B is a modified or unmodified nucleobase, R¹ and R² independently are H, halogen, OR³, or alkyl; and R³ is H, alkyl, cycloalkyl, aryl, aralkyl, heteroaryl or sugar). The term “UNA” refers to unlocked acyclic nucleic acid, wherein any of the bonds of the sugar has been removed, forming an unlocked “sugar” residue. In one example, UNA also encompasses monomers with bonds between C1′-C4′ being removed (i.e., the covalent carbon-oxygen-carbon bond between the C1′ and C4′ carbons). In another example, the C2′-C3′ bond (i.e., the covalent carbon-carbon bond between the C2′ and C3′ carbons) of the sugar is removed (see Mikhailov et. al., Tetrahedron Letters, 26 (17): 2059 (1985); and Fluiter et al., Mol. Biosyst., 10:1039 (2009), which are hereby incorporated by reference in their entirety). The acyclic derivative provides greater backbone flexibility without affecting the Watson-Crick pairings. The acyclic nucleotide can be linked via 2′-5′ or 3′-5′ linkage.

The term ‘GNA’ refers to glycol nucleic acid which is a polymer similar to DNA or RNA but differing in the composition of its “backbone” in that is composed of repeating glycerol units linked by phosphodiester bonds:

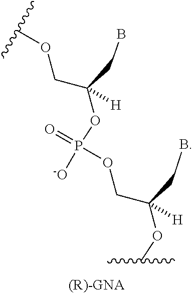

The thermally destabilizing modification of the duplex can be mismatches (i.e., noncomplementary base pairs) between the thermally destabilizing nucleotide and the opposing nucleotide in the opposite strand within the double-stranded region of the dsRNA. Exemplary mismatch base pairs include G:G, G:A, G:U, G:T, A:A, A:C, C:C, C:U, C:T, U:U, T:T, U:T, or a combination thereof. Other mismatch base pairings known in the art are also amenable to the present invention. A mismatch can occur between nucleotides that are either naturally occurring nucleotides or modified nucleotides, i.e., the mismatch base pairing can occur between the nucleobases from respective nucleotides independent of the modifications on the ribose sugars of the nucleotides. In certain embodiments, the dsRNA comprises at least one nucleobase in the mismatch pairing that is a 2′-deoxy nucleobase; e.g., the 2′-deoxy nucleobase is in the sense strand.

In some embodiments, the thermally destabilizing modification in the seed region of the antisense strand includes nucleotides with impaired W—C H-bonding to complementary base on the target mRNA. Exemplary, nucleotides with impaired W—C H-bonding to complementary base on the target mRNA include, but are not limited to, nucleotides comprising a nucleobase independently selected from the following:

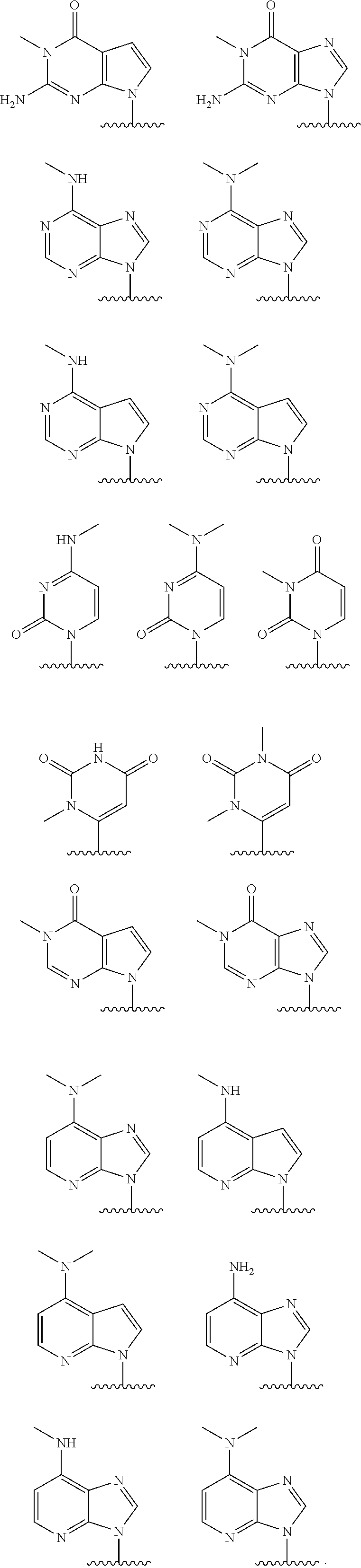

Additional examples of abasic nucleotide, acyclic nucleotide modifications (including UNA and GNA), and mismatch modifications have been described in detail in WO 2011/133876, which is herein incorporated by reference in its entirety.

The thermally destabilizing modifications can also include a universal nucleobase with reduced or abolished capability to form hydrogen bonds with the opposing bases, and phosphate modifications.

In some embodiments, the thermally destabilizing modification includes nucleotides with non-canonical bases such as, but not limited to, nucleobase modifications with impaired or completely abolished capability to form hydrogen bonds with bases in the opposite strand. These nucleobase modifications have been evaluated for destabilization of the central region of the double-stranded region of the dsRNA as described in WO 2010/0011895, which is herein incorporated by reference in its entirety. Exemplary such nucleobase modifications are:

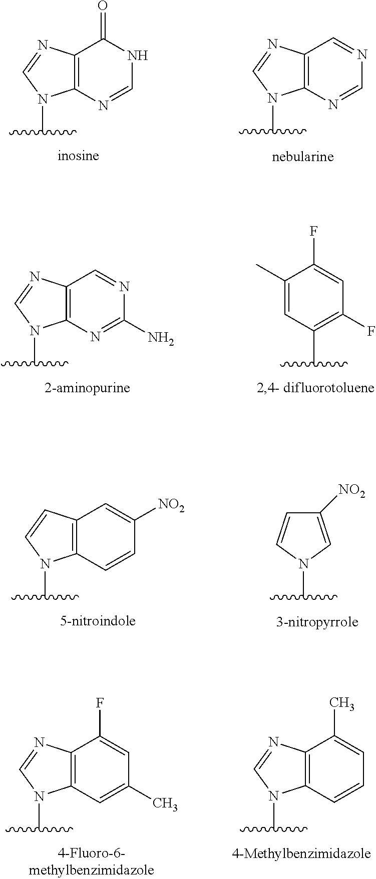

In some embodiments, the thermally destabilizing modification includes one or more α-nucleotides, such as:

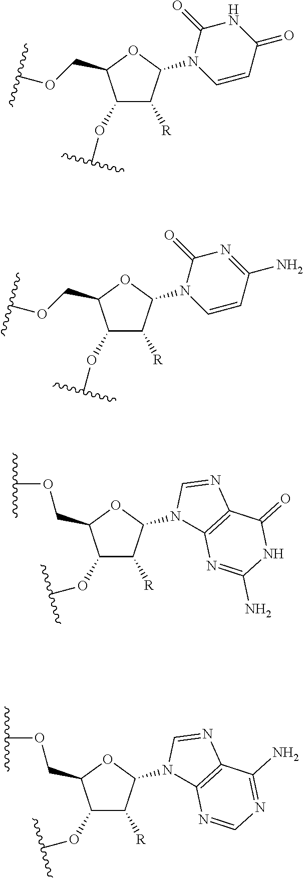

wherein R is H, OH, OCH₃, F, NH₂, NHMe, NMe₂ or O-alkyl

Exemplary phosphate modifications known to decrease the thermal stability of double-stranded nucleic acid duplexes compared to natural phosphodiester linkages include, but are not limited to, the following:

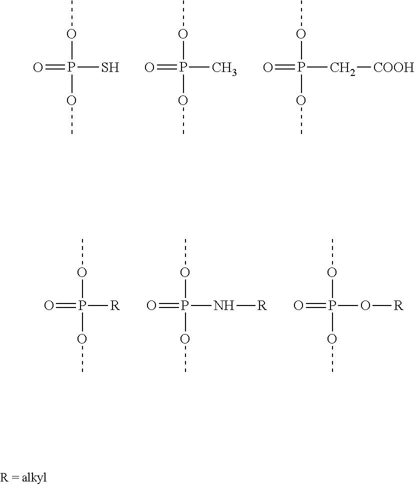

The alkyl for the R group can be a C₁-C₆alkyl. Specific alkyls for the R group include, but are not limited to methyl, ethyl, propyl, isopropyl, butyl, pentyl and hexyl.

In some embodiments, the thermally destabilizing modifications is unlocked (UNA) or glycol nucleic acid (GNA). For example, the thermally destabilizing modifications can include, but are not limited to, mUNA and GNA building blocks as follows:

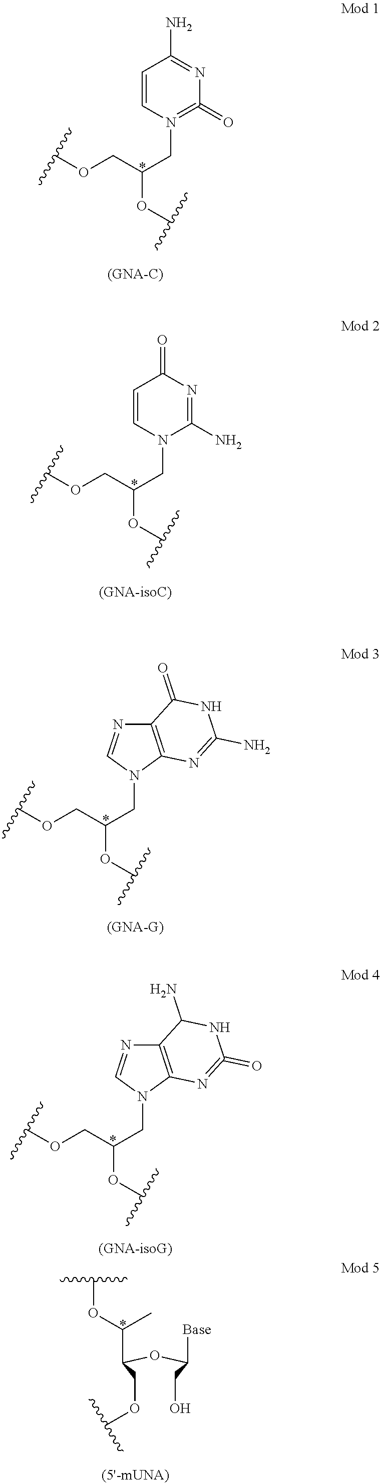

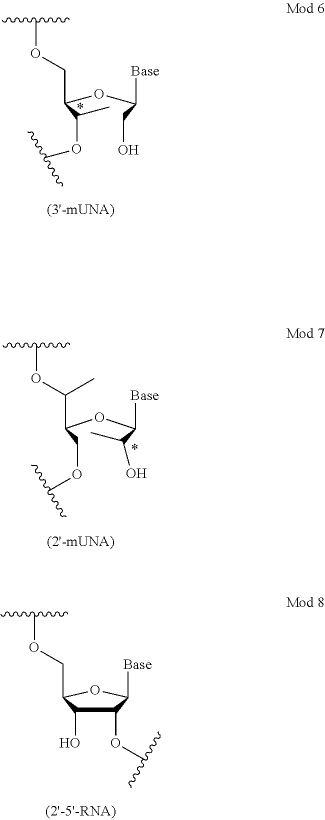

In some embodiments, the destabilizing modification is selected from the following:

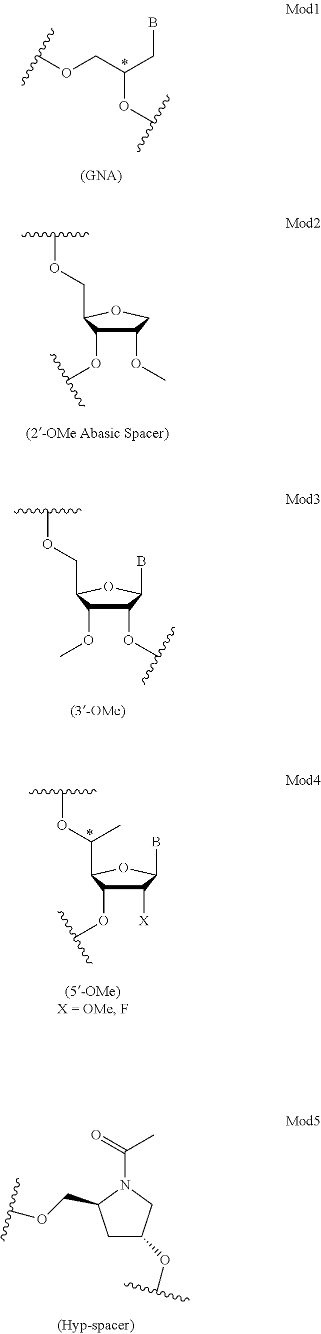

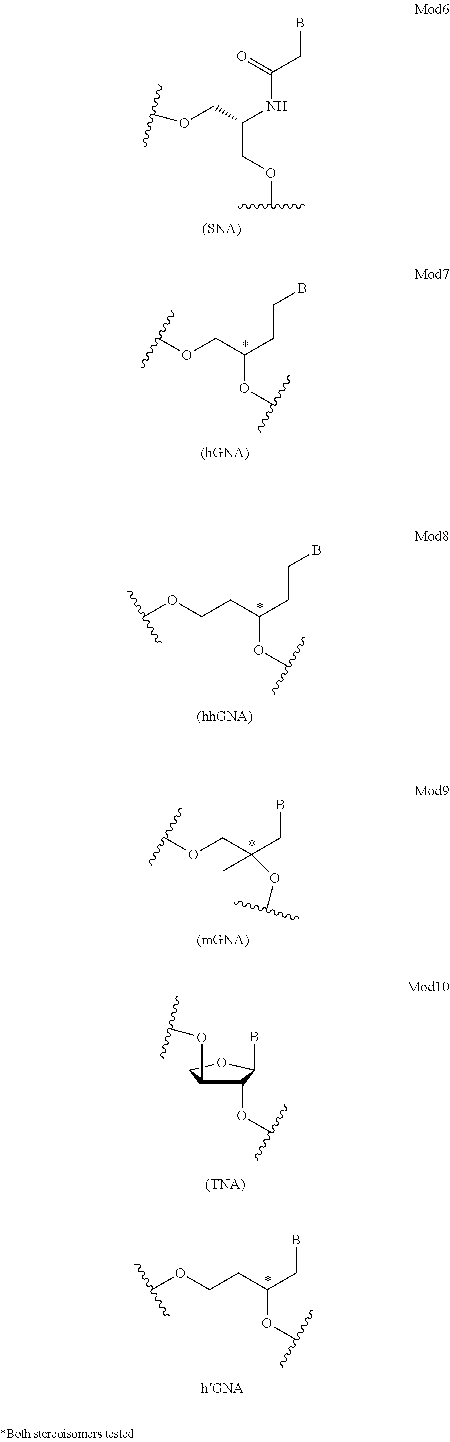

In some embodiments, the destabilizing modification is selected from the following:

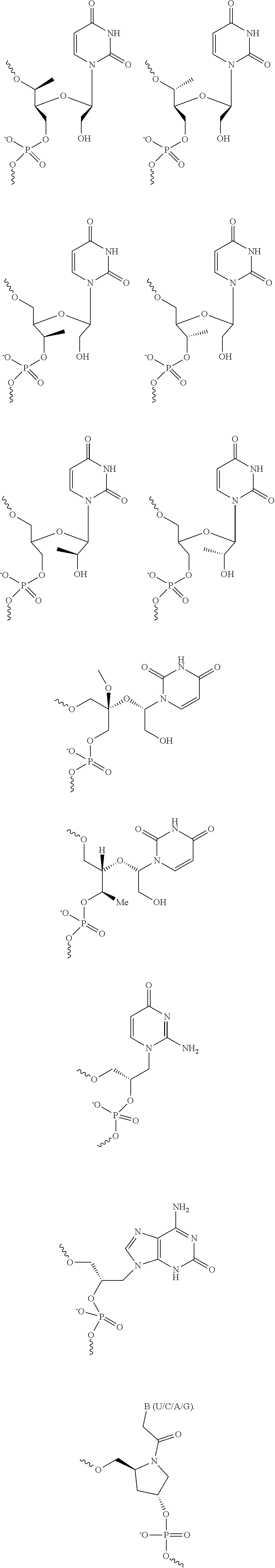

In some embodiments, the destabilizing modification is selected from the following:

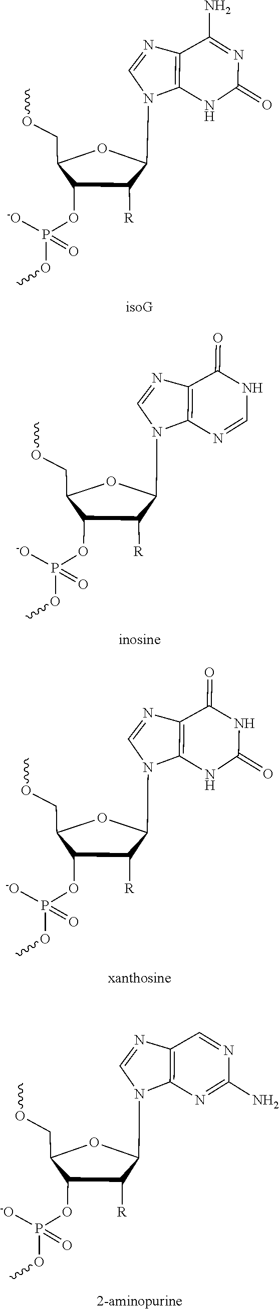

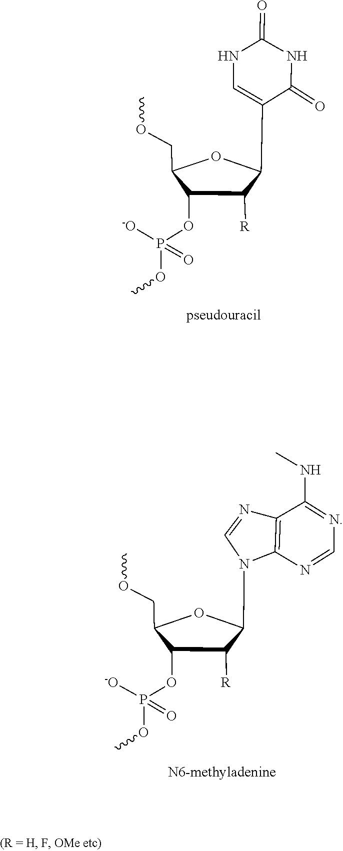

In some embodiments, the destabilizing modification is selected from the group consisting of GNA-isoC, GNA-isoG, 5′-mUNA, 4′-mUNA, 3′-mUNA, and 2′-mUNA.

In some embodiments, the destabilizing modification mUNA is selected from the group consisting of

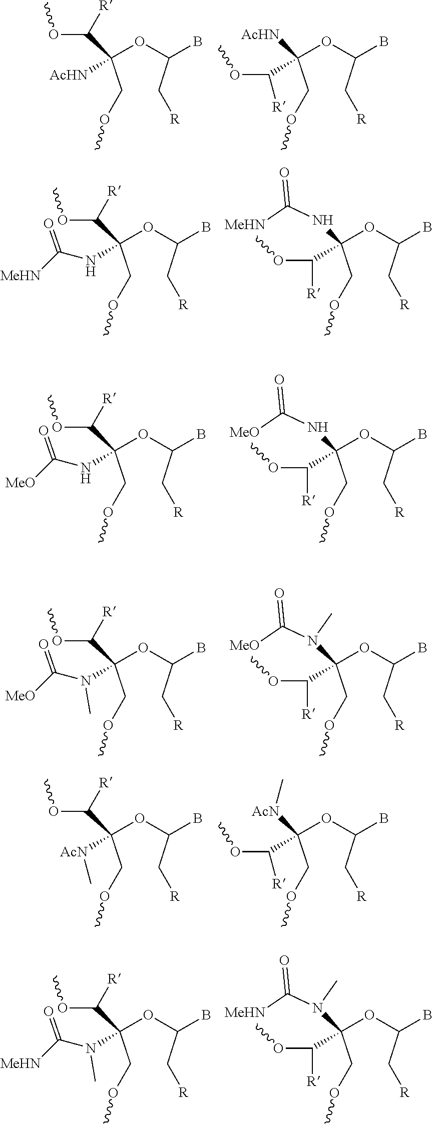

R═H, OH; OMe; Cl, F; OH; O—(CH₂)₂OMe; SMe, NMe₂; NH₂; Me; CCH (alkyne), O-nPr; O-alkyl; O-alkylamino;
R′═H, Me;
B=A; C; 5-Me-C; G; I; U; T; Y; 2-thiouridine; 4-thiouridine; C₅-modified pyrimidines; C₂-modified purines; N8-modiifed purines; phenoxazine; G-clamp; non-canonical mono, bi and tricyclic heterocycles; pseudouracil; isoC; isoG; 2,6-diamninopurine; pseudocytosine; 2-aminopurine; xanthosine; N6-alkyl-A; O6-alkyl-G; 2-thiouridine; 4-thiouridine; C₅-modified pyrimidines; C₂-modified purines; N8-modiifed purines; 7-deazapurines, phenoxazine; G-clamp; non-canonical mono, bi and tricyclic heterocycles; and
Stereochemistry is R or S and combination of R and S for the unspecified chiral centers.

In some embodiments, the destabilizing modification mUNA is selected from the group consisting of

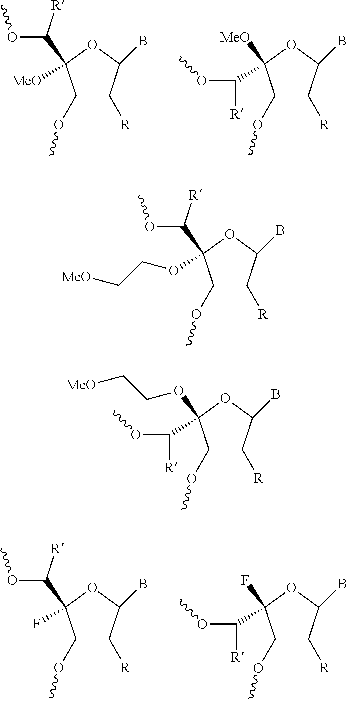

R═H, OH; OMe; Cl, F; OH; O—(CH₂)₂OMe; SMe, NMe₂; NH₂; Me; CCH (alkyne), O-nPr; O-alkyl; O-alkylamino;
R′═H, Me;
B=A; C; 5-Me-C; G; I; U; T; Y; 2-thiouridine; 4-thiouridine; C₅-modified pyrimidines; C₂-modified purines; N8-modiifed purines; phenoxazine; G-clamp; non-canonical mono, bi and tricyclic heterocycles; pseudouracil; isoC; isoG; 2,6-diamninopurine; pseudocytosine; 2-aminopurine; xanthosine; N6-alkyl-A; O6-alkyl-G; 2-thiouridine; 4-thiouridine; C₅-modified pyrimidines; C₂-modified purines; N8-modiifed purines; 7-deazapurines, phenoxazine; G-clamp; non-canonical mono, bi and tricyclic heterocycles; and
Stereochemistry is R or S and combination of R and S for the unspecified chiral centers.

In some embodiments, the destabilizing modification mUNA is selected from the group consisting of

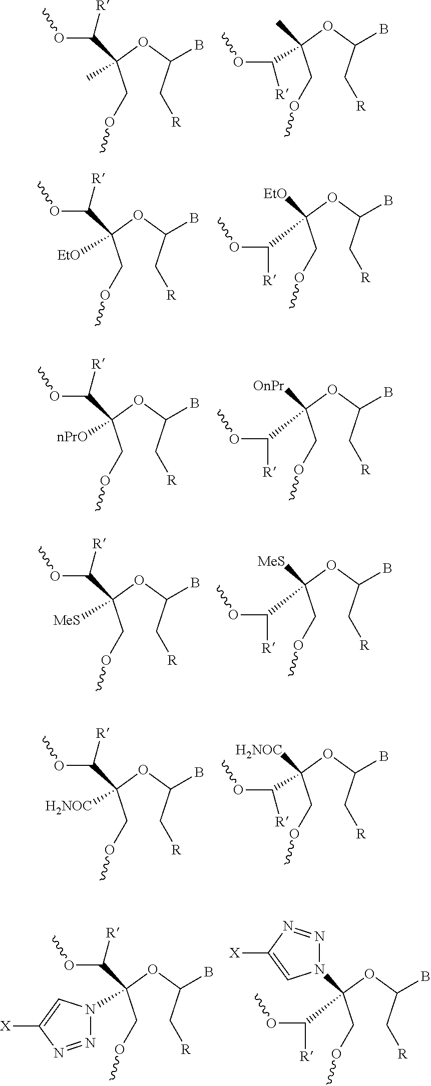

R═H, OMe; F; OH; O—(CH₂)₂OMe; SMe, NMe₂; NH₂; Me; O-nPr; O-alkyl; O-alkylamino;
R′═H, Me;
B=A; C; 5-Me-C; G; I; U; T; Y; 2-thiouridine; 4-thiouridine; C₅-modified pyrimidines; C₂-modified purines; N8-modiifed purines; phenoxazine; G-clamp; non-canonical mono, bi and tricyclic heterocycles; pseudouracil; isoC; isoG; 2,6-diamninopurine; pseudocytosine; 2-aminopurine; xanthosine; N6-alkyl-A; O6-alkyl-G; 7-deazapurines; and
Stereochemistry is R or S and combination of R and S for the unspecified chiral centers.

In some embodiments, the destabilizing modification mUNA is selected from the group consisting of

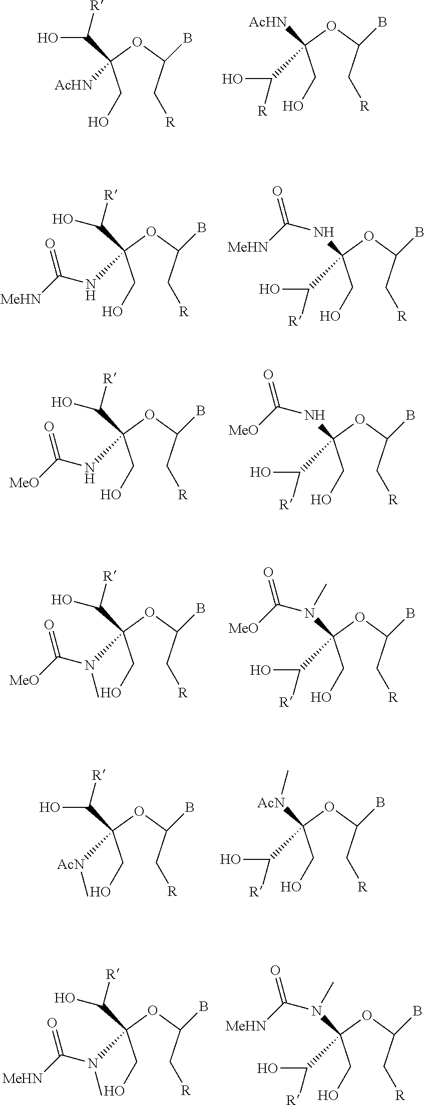

R═H, OH; OMe; Cl, F; OH; O—(CH₂)₂OMe; SMe, NMe₂; NH₂; Me; CCH (alkyne), O-nPr; O-alkyl; O-alkylamino;
R′═H, Me;
B=A; C; 5-Me-C; G; I; U; T; Y; 2-thiouridine; 4-thiouridine; C₅-modified pyrimidines; C₂-modified purines; N8-modified purines; phenoxazine; G-clamp; non-canonical mono, bi and tricyclic heterocycles; pseudouracil; isoC; isoG; 2,6-diamninopurine; pseudocytosine; 2-aminopurine; xanthosine; N6-alkyl-A; O6-alkyl-G; 2-thiouridine; 4-thiouridine; C₅-modified pyrimidines; C2-modified purines; N8-modified purines; 7-deazapurines, phenoxazine; G-clamp; non-canonical mono, bi and tricyclic heterocycles; and
Stereochemistry is R or S and combination of R and S for the unspecified chiral centers

In some embodiments, the destabilizing modification mUNA is selected from the group consisting of

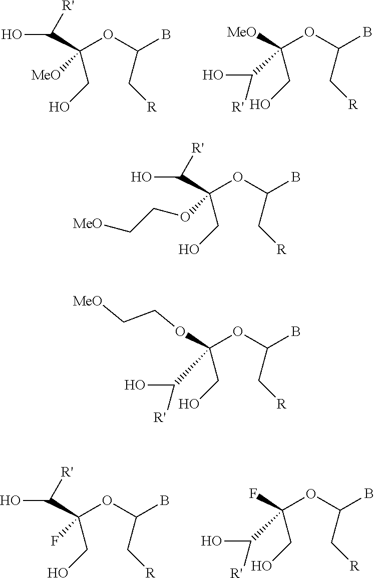

R═H, OH; OMe; C₁, F; OH; O—(CH₂)₂OMe; SMe, NMe₂; NH₂; Me; CCH (alkyne), O-nPr; O-alkyl; O-alkylamino;
R′═H, Me;
B=A; C; 5-Me-C; G; I; U; T; Y; 2-thiouridine; 4-thiouridine; C₅-modified pyrimidines; C₂-modified purines; N8-modified purines; phenoxazine; G-clamp; non-canonical mono, bi and tricyclic heterocycles; pseudouracil; isoC; isoG; 2,6-diamninopurine; pseudocytosine; 2-aminopurine; xanthosine; N6-alkyl-A; O6-alkyl-G; 2-thiouridine; 4-thiouridine; C₅-modified pyrimidines; C₂-modified purines; N8-modified purines; 7-deazapurines, phenoxazine; G-clamp; non-canonical mono, bi and tricyclic heterocycles; and
Stereochemistry is R or S and combination of R and S for the unspecified chiral centers

In some embodiments, the modification mUNA is selected from the group consisting of

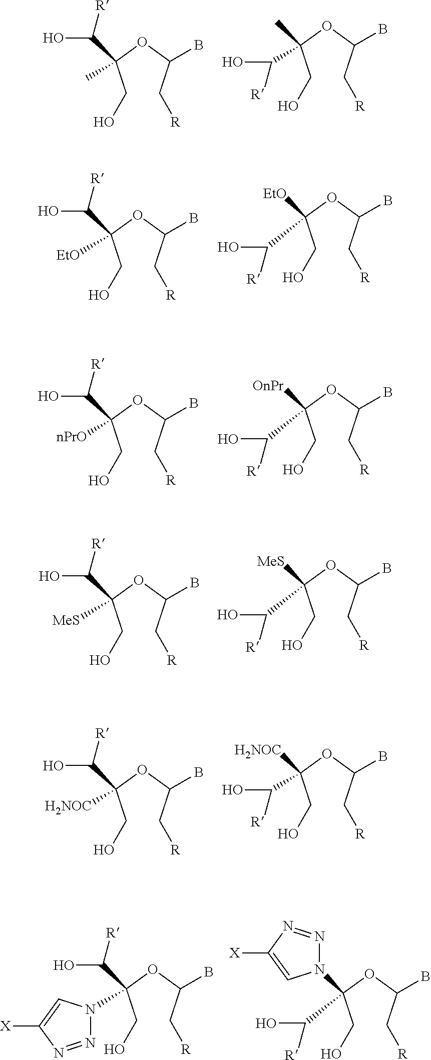

R═H, OMe; F; OH; O—(CH₂)₂OMe; SMe, NMe₂; NH₂; Me; O-nPr; O-alkyl; O-alkylamino;
R′═H, Me;
B=A; C; 5-Me-C; G; I; U; T; Y; 2-thiouridine; 4-thiouridine; C₅-modified pyrimidines; C₂-modified purines; N8-modified purines; phenoxazine; G-clamp; non-canonical mono, bi and tricyclic heterocycles; pseudouracil; isoC; isoG; 2,6-diamninopurine; pseudocytosine; 2-aminopurine; xanthosine; N6-alkyl-A; O6-alkyl-G; 7-deazapurines; and
Stereochemistry is R or S and combination of R and S for the unspecified chiral centers

In some embodiments, the antisense strand comprises at least two (e.g., two, three, four, five, six, seven, eight, nine, ten or more) stabilizing modifications. Without limitations, a stabilizing modification in the antisense strand can be present at any positions. In some embodiments, the antisense strand comprises stabilizing modifications at positions 2, 6, 8, 9, 14 and 16, counting from the 5′-end. In some other embodiments, the antisense strand comprises stabilizing modifications at positions 2, 6, 14 and 16, counting from the 5′-end. In still some other embodiments, the antisense strand comprises stabilizing modifications at positions 2, 14 and 16, counting from the 5′-end. In some embodiments, the antisense strand comprises stabilizing modifications at positions 7, 10 and 11, counting from the 5′-end. In some other embodiments, the antisense strand comprises stabilizing modifications at positions 7, 9, 10 and 11, counting from the 5′-end.

In some embodiments, the antisense strand comprises at least one stabilizing modification adjacent to the destabilizing modification. For example, the stabilizing modification can be the nucleotide at the 5′-end or the 3′-end of the destabilizing modification, i.e., at position −1 or +1 from the position of the destabilizing modification. In some embodiments, the antisense strand comprises a stabilizing modification at each of the 5′-end and the 3′-end of the destabilizing modification, i.e., positions-1 and +1 from the position of the destabilizing modification.

In some embodiments, the antisense strand comprises at least two stabilizing modifications at the 3′-end of the destabilizing modification, i.e., at positions +1 and +2 from the position of the destabilizing modification.

In some embodiments, the sense strand does not comprise a thermally stabilizing modification in position opposite or complimentary to the thermally destabilizing modification of the duplex in the antisense strand.

In some embodiments, the antisense strand comprises at least one 2′-fluoro nucleotide adjacent to the destabilizing modification. For example, the 2′-fluoro nucleotide can be the nucleotide at the 5′-end or the 3′-end of the destabilizing modification, i.e., at position-1 or +1 from the position of the destabilizing modification. In some embodiments, the antisense strand comprises a 2′-fluoro nucleotide at each of the 5′-end and the 3′-end of the destabilizing modification, i.e., positions-1 and +1 from the position of the destabilizing modification.

In some embodiments, the antisense strand comprises at least two 2′-fluoro nucleotides at the 3′-end of the destabilizing modification, i.e., at positions +1 and +2 from the position of the destabilizing modification.

In some embodiments, the sense strand does not comprise a 2′-fluoro nucleotide in position opposite or complimentary to the thermally destabilizing modification of the duplex in the antisense strand.

In some embodiments, every nucleotide in the sense strand and/or the antisense strand can be modified. Each nucleotide can be modified with the same or different modification which can include one or more alteration of one or both of the non-linking phosphate oxygens and/or of one or more of the linking phosphate oxygens; alteration of a constituent of the ribose sugar, e.g., of the 2′ hydroxyl on the ribose sugar; wholesale replacement of the phosphate moiety with “dephospho” linkers; modification or replacement of a naturally occurring base; and replacement or modification of the ribose-phosphate backbone.

As nucleic acids are polymers of monomers, many of the modifications occur at a position which is repeated within a nucleic acid, e.g., a modification of a base, or a phosphate moiety, or a non-linking O of a phosphate moiety. In some cases, the modification will occur at all of the subject positions in the nucleic acid but in many cases it will not. By way of example, a modification may only occur at a 3′ or 5′ terminal position, may only occur in a terminal region, e.g., at a position on a terminal nucleotide or in the last 2, 3, 4, 5, or 10 nucleotides of a strand. A modification may occur in a double strand region, a single strand region, or in both. A modification may occur only in the double strand region of an RNA or may only occur in a single strand region of an RNA. For example, a phosphorothioate modification at a non-linking O position may only occur at one or both termini, may only occur in a terminal region, e.g., at a position on a terminal nucleotide or in the last 2, 3, 4, 5, or 10 nucleotides of a strand, or may occur in double strand and single strand regions, particularly at termini. The 5′ end or ends can be phosphorylated.

It may be possible, e.g., to enhance stability, to include particular bases in overhangs, or to include modified nucleotides or nucleotide surrogates, in single strand overhangs, e.g., in a 5′ or 3′ overhang, or in both. E.g., it can be desirable to include purine nucleotides in overhangs. In some embodiments all or some of the bases in a 3′ or 5′ overhang may be modified, e.g., with a modification described herein. Modifications can include, e.g., the use of modifications at the 2′ position of the ribose sugar with modifications that are known in the art, e.g., the use of deoxyribonucleotides, 2′-deoxy-2′-fluoro (2′-F) or 2′-O-methyl modified instead of the ribosugar of the nucleobase, and modifications in the phosphate group, e.g., phosphorothioate modifications. Overhangs need not be homologous or orthologous with the target sequence.

In some embodiments, each residue of the sense strand and/or antisense strand is independently modified with LNA, HNA, CeNA, 2′-methoxyethyl, 2′-O-methyl, 2′-O-allyl, 2′-C-allyl, 2′-deoxy, or 2′-fluoro. The strands can contain more than one modification. In some embodiments, each residue of the sense strand and antisense strand is independently modified with 2′-O-methyl or 2′-fluoro. It is to be understood that these modifications are in addition to the at least one thermally destabilizing modification of the duplex present in the antisense strand.

At least two different modifications are typically present on the sense strand and antisense strand. Those two modifications may be the 2′-deoxy, 2′-O-methyl or 2′-fluoro modifications, acyclic nucleotides or others. In some embodiments, the sense strand and antisense strand each comprises two differently modified nucleotides selected from 2′-O-methyl or 2′-deoxy. In some embodiments, each residue of the sense strand and antisense strand is independently modified with a 2′-O-methyl nucleotide, 2′-deoxy nucleotide, 2′-deoxy-2′-fluoro nucleotide, 2′-O—N-methylacetamido (2′-O-NMA) nucleotide, a 2′-O-dimethylaminoethoxyethyl (2′-O-DMAEOE) nucleotide, 2′-O-aminopropyl (2′-O-AP) nucleotide, or 2′-ara-F nucleotide. Again, it is to be understood that these modifications are in addition to the at least one thermally destabilizing modification of the duplex present in the antisense strand.

In some embodiments, the oligonucleotide or dsRNA described herein comprises modifications of an alternating pattern, particular in the B1, B2, B3, B1′, B2′, B3′, B4′ regions. The term “alternating motif” or “alternative pattern” as used herein refers to a motif having one or more modifications, each modification occurring on alternating nucleotides of one strand. The alternating nucleotide may refer to one per every other nucleotide or one per every three nucleotides, or a similar pattern. For example, if A, B and C each represent one type of modification to the nucleotide, the alternating motif can be “ABABABABABAB . . . ,”

“AABBAABBAABB . . . ,” “AABAABAABAAB . . . ,” “AAABAAABAAAB . . . ,”

“AAABBBAAABBB . . . ,” or “ABCABCABCABC . . . ,” etc.

The type of modifications contained in the alternating motif may be the same or different. For example, if A, B, C, D each represent one type of modification on the nucleotide, the alternating pattern, i.e., modifications on every other nucleotide, may be the same, but each of the sense strand or antisense strand can be selected from several possibilities of modifications within the alternating motif such as “ABABAB . . . ”, “ACACAC . . . ” “BDBDBD . . . ” or “CDCDCD . . . ,” etc.

In some embodiments, the dsRNA comprises the modification pattern for the alternating motif on the sense strand relative to the modification pattern for the alternating motif on the antisense strand is shifted. The shift may be such that the modified group of nucleotides of the sense strand corresponds to a differently modified group of nucleotides of the antisense strand and vice versa. For example, the sense strand when paired with the antisense strand in the dsRNA, the alternating motif in the sense strand may start with “ABABAB” from 5′-3′ of the strand and the alternating motif in the antisense strand may start with “BABABA” from 3′-5′ of the strand within the duplex region. As another example, the alternating motif in the sense strand may start with “AABBAABB” from 5′-3′ of the strand and the alternating motif in the antisense strand may start with “BBAABBAA” from 3′-5′ of the strand within the duplex region, so that there is a complete or partial shift of the modification patterns between the sense strand and the antisense strand.

In some embodiments, the oligonucleotide or dsRNA described herein comprises mismatch(es) with the target, within the duplex, or combinations thereof. The mismatch can occur in the overhang region or the duplex region. The base pair can be ranked on the basis of their propensity to promote dissociation or melting (e.g., on the free energy of association or dissociation of a particular pairing, the simplest approach is to examine the pairs on an individual pair basis, though next neighbor or similar analysis can also be used). In terms of promoting dissociation: A:U is preferred over G:C; G:U is preferred over G:C; and I:C is preferred over G: C (I-inosine). Mismatches, e.g., non-canonical or other than canonical pairings (as described elsewhere herein) are preferred over canonical (A: T, A: U, G: C) pairings; and pairings which include a universal base are preferred over canonical pairings.

In some embodiments, the dsRNA comprises at least one of the first 1, 2, 3, 4, or 5 base pairs within the duplex regions from the 5′-end of the antisense strand can be chosen independently from the group of: A:U, G:U, I:C, and mismatched pairs, e.g., non-canonical or other than canonical pairings or pairings which include a universal base, to promote the dissociation of the antisense strand at the 5′-end of the duplex.

In some embodiments, the nucleotide at the 1 position within the duplex region from the 5′-end in the antisense strand is selected from the group consisting of A, dA, dU, U, and dT. Alternatively, at least one of the first 1, 2 or 3 base pair within the duplex region from the 5′-end of the antisense strand is an AU base pair. For example, the first base pair within the duplex region from the 5′-end of the antisense strand is an AU base pair.

Without wishing to be bound by a theory, introducing 4′-modified and/or 5′-modified nucleotides to the 3′-end of a phosphodiester (PO), phosphorothioate (PS), and/or phosphorodithioate (PS2) linkage of a dinucleotide at any position of single stranded or double stranded nucleic acid can exert steric effect to the internucleotide linkage and, hence, protecting or stabilizing it against nucleases.

In some embodiments, a 5′-modified nucleoside is introduced at the 3′-end of a dinucleotide at any position of the sense and/or the antisense strand. For instance, a 5′-alkylated nucleoside can be introduced at the 3′-end of a dinucleotide at any position of the sense and/or the antisense strand. The alkyl group at the 5′ position of the ribose sugar can be a racemic or enantiomerically pure R or S isomer. An exemplary 5′-alkylated nucleoside is a 5′-methyl nucleoside. The 5′-methyl can be either a racemic or enantiomerically pure R or S isomer.

In some embodiments, a 4′-modified nucleoside is introduced at the 3′-end of a dinucleotide at any position of the sense and/or the antisense strand. For instance, a 4′-alkylated nucleoside may be introduced at the 3′-end of a dinucleotide at any position the sense and/or the antisense strand. The alkyl group at the 4′ position of the ribose sugar can be a racemic or enantiomerically pure R or S isomer. An exemplary 4′-alkylated nucleoside is a 4′-methyl nucleoside. The 4′-methyl can be either racemic or enantiomerically pure R or S isomer. Alternatively, a 4′-O-alkylated nucleoside may be introduced at the 3′-end of a dinucleotide at any position of the sense and/or the antisense strand. The 4′-O-alkyl of the ribose sugar can be a racemic or enantiomerically pure R or S isomer. An exemplary 4′-O-alkylated nucleoside is a 4′-O-methyl nucleoside. The 4′-O-methyl can be either a racemic or enantiomerically pure R or S isomer.

In some embodiments, a 5′-alkylated nucleoside is introduced at any position on the sense strand or antisense strand of the sense and/or the antisense strand, and such modification maintains or improves potency of the double-stranded nucleic acid. The 5′-alkyl can be either a racemic or enantiomerically pure R or S isomer. An exemplary 5′-alkylated nucleoside is a 5′-methyl nucleoside. The 5′-methyl can be either a racemic or enantiomerically pure R or S isomer.

In some embodiments, a 4′-alkylated nucleoside is introduced at any position on the sense strand or antisense strand of the dsRNA, and such modification maintains or improves potency of the dsRNA. The 4′-alkyl can be either a racemic or enantiomerically pure R or Sisomer.

An exemplary 4′-alkylated nucleoside is a 4′-methyl nucleoside. The 4′-methyl can be either a racemic or enantiomerically pure R or S isomer.

In some embodiments, a 4′-O-alkylated nucleoside is introduced at any position on the sense strand or antisense strand of the dsRNA, and such modification maintains or improves potency of the dsRNA. The 5′-alkyl can be either a racemic or enantiomerically pure R or Sisomer. An exemplary 4′-O-alkylated nucleoside is a 4′-O-methyl nucleoside. The 4′-O-methyl can be either a racemic or enantiomerically pure R or S isomer.

In some embodiments, the oligonucleotide or dsRNA described herein can comprise 2′-5′ linkages (with 2′-H, 2′-OH and 2′-OMe and with P—O or P═S). For example, the 2′-5′ linkages modifications can be used to promote nuclease resistance or to inhibit binding of the sense to the antisense strand, or can be used at the 5′ end of the sense strand to avoid sense strand activation by RISC. In some embodiments, the sense strand comprises a 2′-5′-linkage between positions N-1 and N-2, counting from 5′-end.

In some embodiments, the oligonucleotide or dsRNA described herein dsRNA can comprise L sugars (e.g., L ribose, L-arabinose with 2′-H, 2′-OH and 2′-OMe). For example, these L sugars modifications can be used to promote nuclease resistance or to inhibit binding of the sense to the antisense strand, or can be used at the 5′ end of the sense strand to avoid sense strand activation by RISC. In some embodiments, the sense strand comprises a L sugar nucleotide at the 5′-end.

### Ligands

Embodiments of the various aspects described herein include a ligand. Without wishing to be bound by a theory, ligands modify one or more properties of the attached molecule (e.g., the oligonucleotide described herein) including but not limited to pharmacodynamic, pharmacokinetic, binding, absorption, cellular distribution, cellular uptake, charge and clearance. Ligands are routinely used in the chemical arts and are linked directly or via an optional linking moiety or linking group to a parent compound. A preferred list of ligands includes without limitation, intercalators, reporter molecules, polyamines, polyamides, polyethylene glycols, thioethers, polyethers, cholesterols, thiocholesterols, cholic acid moieties, folate, lipids, phospholipids, biotin, phenazine, phenanthridine, anthraquinone, adamantane, acridine, fluoresceins, rhodamines, coumarins and dyes.

Preferred ligands amenable to the present invention include lipid moieties such as a cholesterol moiety (Letsinger et al., Proc. Natl. Acad. Sci. USA, 1989, 86, 6553); cholic acid (Manoharan et al., Bioorg. Med. Chem. Lett., 1994, 4, 1053); a thioether, e.g., hexyl-S-tritylthiol (Manoharan et al., Ann. N.Y. Acad. Sci., 1992, 660, 306; Manoharan et al., Bioorg. Med. Chem. Let., 1993, 3, 2765); a thiocholesterol (Oberhauser et al., Nucl. Acids Res., 1992, 20, 533); an aliphatic chain, e.g., dodecandiol or undecyl residues (Saison-Behmoaras et al., EMBO J., 1991, 10, 111; Kabanov et al., FEBS Lett., 1990, 259, 327; Svinarchuk et al., Biochimie, 1993, 75, 49); a phospholipid, e.g., di-hexadecyl-rac-glycerol or triethylammonium-1,2-di-O-hexadecyl-rac-glycero-3-H-phosphonate (Manoharan et al., Tetrahedron Lett., 1995, 36, 3651; Shea et al., Nucl. Acids Res., 1990, 18, 3777); a polyamine or a polyethylene glycol chain (Manoharan et al., Nucleosides & Nucleotides, 1995, 14, 969); adamantane acetic acid (Manoharan et al., Tetrahedron Lett., 1995, 36, 3651); a palmityl moiety (Mishra et al., Biochim. Biophys. Acta, 1995, 1264, 229); or an octadecylamine or hexylamino-carbonyl-oxycholesterol moiety (Crooke et al., J. Pharmacol. Exp. Ther., 1996, 277, 923).

Ligands can include naturally occurring molecules, or recombinant or synthetic molecules. Exemplary ligands include, but are not limited to, polylysine (PLL), poly L-aspartic acid, poly L-glutamic acid, styrene-maleic acid anhydride copolymer, poly (L-lactide-co-glycolied) copolymer, divinyl ether-maleic anhydride copolymer, N-(2-hydroxylpropyl) methacrylamide copolymer (HMPA), polyethylene glycol (PEG, e.g., PEG-2K, PEG-5K, PEG-10K, PEG-12K, PEG-15K, PEG-20K, PEG-40K), MPEG, [MPEG]₂, polyvinyl alcohol (PVA), polyurethane, poly(2-ethylacryllic acid), N-isopropylacrylamide polymers, polyphosphazine, polyethylenimine, cationic groups, spermine, spermidine, polyamine, pseudopeptide-polyamine, peptidomimetic polyamine, dendrimer polyamine, arginine, amidine, protamine, cationic lipid, cationic porphyrin, quaternary salt of a polyamine, thyrotropin, melanotropin, lectin, glycoprotein, surfactant protein A, mucin, glycosylated polyaminoacids, transferrin, bisphosphonate, polyglutamate, polyaspartate, aptamer, asialofetuin, hyaluronan, procollagen, immunoglobulins (e.g., antibodies), insulin, transferrin, albumin, sugar-albumin conjugates, intercalating agents (e.g., acridines), cross-linkers (e.g. psoralen, mitomycin C), porphyrins (e.g., TPPC4, texaphyrin, Sapphyrin), polycyclic aromatic hydrocarbons (e.g., phenazine, dihydrophenazine), artificial endonucleases (e.g., EDTA), lipophilic molecules (e.g, steroids, bile acids, cholesterol, cholic acid, adamantane acetic acid, 1-pyrene butyric acid, dihydrotestosterone, 1,3-Bis-O(hexadecyl)glycerol, geranyloxyhexyl group, hexadecylglycerol, borneol, menthol, 1,3-propanediol, heptadecyl group, palmitic acid, myristic acid,O3-(oleoyl) lithocholic acid, O3-(oleoyl) cholenic acid, dimethoxytrityl, or phenoxazine), peptides (e.g., an alpha helical peptide, amphipathic peptide, RGD peptide, cell permeation peptide, endosomolytic/fusogenic peptide), alkylating agents, phosphate, amino, mercapto, polyamino, alkyl, substituted alkyl, radiolabeled markers, enzymes, haptens (e.g. biotin), transport/absorption facilitators (e.g., naproxen, aspirin, vitamin E, folic acid), synthetic ribonucleases (e.g., imidazole, bisimidazole, histamine, imidazole clusters, acridine-imidazole conjugates, Eu3+ complexes of tetraazamacrocycles), dinitrophenyl, HRP, AP, antibodies, hormones and hormone receptors, lectins, carbohydrates, multivalent carbohydrates, vitamins (e.g., vitamin A, vitamin E, vitamin K, vitamin B, e.g., folic acid, B12, riboflavin, biotin and pyridoxal), vitamin cofactors, lipopolysaccharide, an activator of p38 MAP kinase, an activator of NF-κB, taxon, vincristine, vinblastine, cytochalasin, nocodazole, japlakinolide, latrunculin A, phalloidin, swinholide A, indanocine, myoservin, tumor necrosis factor alpha (TNFalpha), interleukin-1 beta, gamma interferon, natural or recombinant low density lipoprotein (LDL), natural or recombinant high-density lipoprotein (HDL), and a cell-permeation agent (e.g., a.helical cell-permeation agent).

Peptide and peptidomimetic ligands include those having naturally occurring or modified peptides, e.g., D or L peptides; a, B, or y peptides; N-methyl peptides; azapeptides; peptides having one or more amide, i.e., peptide, linkages replaced with one or more urea, thiourea, carbamate, or sulfonyl urea linkages; or cyclic peptides. A peptidomimetic (also referred to herein as an oligopeptidomimetic) is a molecule capable of folding into a defined three-dimensional structure similar to a natural peptide. The peptide or peptidomimetic ligand can be about 5-50 amino acids long, e.g., about 5, 10, 15, 20, 25, 30, 35, 40, 45, or 50 amino acids long.

Exemplary amphipathic peptides include, but are not limited to, cecropins, lycotoxins, paradaxins, buforin, CPF, bombinin-like peptide (BLP), cathelicidins, ceratotoxins, S. clava peptides, hagfish intestinal antimicrobial peptides (HFIAPs), magainines, brevinins-2, dermaseptins, melittins, pleurocidin, H₂A peptides, Xenopus peptides, esculentinis-1, and caerins.

As used herein, the term “endosomolytic ligand” refers to molecules having endosomolytic properties. Endosomolytic ligands promote the lysis of and/or transport of the composition of the invention, or its components, from the cellular compartments such as the endosome, lysosome, endoplasmic reticulum (ER), Golgi apparatus, microtubule, peroxisome, or other vesicular bodies within the cell, to the cytoplasm of the cell. Some exemplary endosomolytic ligands include, but are not limited to, imidazoles, poly or oligoimidazoles, linear or branched polyethyleneimines (PEIs), linear and brached polyamines, e.g. spermine, cationic linear and branched polyamines, polycarboxylates, polycations, masked oligo or poly cations or anions, acetals, polyacetals, ketals/polyketals, orthoesters, linear or branched polymers with masked or unmasked cationic or anionic charges, dendrimers with masked or unmasked cationic or anionic charges, polyanionic peptides, polyanionic peptidomimetics, pH-sensitive peptides, natural and synthetic fusogenic lipids, natural and synthetic cationic lipids.

Exemplary endosomolytic/fusogenic peptides include, but are not limited to, AALEALAEALEALAEALEALAEAAAAGGC (GALA) (SEQ ID NO.: 1);

AALAEALAEALAEALAEALAEALAAAAGGC (EALA) (SEQ ID NO.: 2);

ALEALAEALEALAEA (SEQ ID NO.: 3); GLFEAIEGFIENGWEGMIWDYG (INF-7) (SEQ ID NO.: 4); GLFGAIAGFIENGWEGMIDGWYG (Inf HA-2) (SEQ ID NO.: 5);

GLFEAIEGFIENGWEGMIDGWYGCGLFEAIEGFIENGWEGMID GWYGC (diINF-7) (SEQ ID NO.: 6); GLFEAIEGFIENGWEGMIDGGCGLFEAIEGFIENGWEGMIDGGC (diINF-3) (SEQ ID NO.: 7); GLFGALAEALAEALAEHLAEALAEALEALAAGGSC (GLF) (SEQ ID NO.:
8); GLFEAIEGFIENGWEGLAEALAEALEALAAGGSC (GALA-INF3) (SEQ ID NO.: 9); GLF EAI EGFI ENGW EGnI DG K GLF EAI EGFI ENGW EGnI DG (INF-5, n is norleucine) (SEQ ID NO.: 10); LFEALLELLESLWELLLEA (JTS-1) (SEQ ID NO.: 11); GLFKALLKLLKSLWKLLLKA (ppTG1) (SEQ ID NO.: 12); GLFRALLRLLRSLWRLLLRA (ppTG20) (SEQ ID NO.: 13); WEAKLAKALAKALAKHLAKALAKALKACEA (KALA) (SEQ ID NO.: 14); GLFFEAIAEFIEGGWEGLIEGC (HA) (SEQ ID NO.: 15); GIGAVLKVLTTGLPALISWIKRKRQQ (Melittin) (SEQ ID NO.: 16); HsWYG (SEQ ID NO.: 17); and CHK&HC (SEQ ID NO.: 18).

Without wishing to be bound by theory, fusogenic lipids fuse with and consequently destabilize a membrane. Fusogenic lipids usually have small head groups and unsaturated acyl chains. Exemplary fusogenic lipids include, but are not limited to, 1,2-dileoyl-sn-3-phosphoethanolamine (DOPE), phosphatidylethanolamine (POPE), palmitoyloleoylphosphatidylcholine (POPC), (6Z,9Z,28Z,31Z)-heptatriaconta-6,9,28,31-tetraen-19-ol (Di-Lin), N-methyl (2,2-di((9Z,12Z)-octadeca-9,12-dienyl)-1,3-dioxolan-4-yl) methanamine (DLin-k-DMA) and N-methyl-2-(2,2-di((9Z,12Z)-octadeca-9,12-dienyl)-1,3-dioxolan-4-yl) ethanamine (also refered to as XTC herein).

Synthetic polymers with endosomolytic activity amenable to the present invention are described in U.S. Pat. App. Pub. Nos. 2009/0048410; 2009/0023890; 2008/0287630; 2008/0287628; 2008/0281044; 2008/0281041; 2008/0269450; 2007/0105804; 20070036865; and 2004/0198687, contents of which are hereby incorporated by reference in their entirety.

Exemplary cell permeation peptides include, but are not limited to, RQIKIWFQNRRMKWKK (penetratin) (SEQ ID NO.: 19); GRKKRRQRRRPPQC (Tat fragment 48-60) (SEQ ID NO.: 20); GALFLGWLGAAGSTMGAWSQPKKKRKV (signal sequence based peptide) (SEQ ID NO.: 21); LLIILRRRIRKQAHAHSK (PVEC) (SEQ ID NO.: 22); GWTLNSAGYLLKINLKALAALAKKIL (SEQ (transportan) ID NO.: 23); KLALKLALKALKAALKLA (amphiphilic model peptide) (SEQ ID NO.: 24); RRRRRRRRR (Arg9) (SEQ ID NO.: 25); KFFKFFKFFK (Bacterial cell wall permeating peptide) (SEQ ID NO.: 26); LLGDFFRKSKEKIGKEFKRIVQRIKDFLRNLVPRTES (LL-37) (SEQ ID NO.: 27); SWLSKTAKKLENSAKKRISEGIAIAIQGGPR (cecropin P1) (SEQ ID NO.: 28);
ACYCRIPACIAGERRYGTCIYQGRLWAFCC (α-defensin) (SEQ ID NO.: 29);
DHYNCVSSGGQCLYSACPIFTKIQGTCYRGKAKCCK (β-defensin) (SEQ ID NO.: 30); RRRPRPPYLPRPRPPPFFPPRLPPRIPPGFPPRFPPRFPGKR-NH₂ (PR-39) (SEQ ID NO.: 31;
ILPWKWPWWPWRR-NH₂ (indolicidin) (SEQ ID NO.: 32); AAVALLPAVLLALLAP (RFGF) (SEQ ID NO.: 33); AALLPVLLAAP (RFGF analogue) (SEQ ID NO.: 34); and RKCRIVVIRVCR (bactenecin) (SEQ ID NO.: 35).

Exemplary cationic groups include, but are not limited to, protonated amino groups, derived from e.g., O-AMINE (AMINE=NH₂; alkylamino, dialkylamino, heterocyclyl, arylamino, diaryl amino, heteroaryl amino, or diheteroaryl amino, ethylene diamine, polyamino); aminoalkoxy, e.g., O(CH₂) nAMINE, (e.g., AMINE=NH₂; alkylamino, dialkylamino, heterocyclyl, arylamino, diaryl amino, heteroaryl amino, or diheteroaryl amino, ethylene diamine, polyamino); amino (e.g. NH₂; alkylamino, dialkylamino, heterocyclyl, arylamino, diaryl amino, heteroaryl amino, diheteroaryl amino, or amino acid); and NH(CH₂CH₂NH)ₙCH₂CH₂-AMINE (AMINE=NH₂; alkylamino, dialkylamino, heterocyclyl, arylamino, diaryl amino, heteroaryl amino, or diheteroaryl amino).

As used herein the term “targeting ligand” refers to any molecule that provides an enhanced affinity for a selected target, e.g., a cell, cell type, tissue, organ, region of the body, or a compartment, e.g., a cellular, tissue or organ compartment. Some exemplary targeting ligands include, but are not limited to, antibodies, antigens, folates, receptor ligands, carbohydrates, aptamers, integrin receptor ligands, chemokine receptor ligands, transferrin, biotin, serotonin receptor ligands, PSMA, endothelin, GCPII, somatostatin, LDL and HDL ligands.

Carbohydrate based targeting ligands include, but are not limited to, D-galactose, multivalent galactose, N-acetyl-D-galactosamine (GalNAc), multivalent GalNAc, e.g. GalNAc2 and GalNAc3; D-mannose, multivalent mannose, multivalent lactose, N-acetyl-gulucosamine, multivalent fucose, glycosylated polyaminoacids and lectins. The term multivalent indicates that more than one monosaccharide unit is present. Such monosaccharide subunits can be linked to each other through glycosidic linkages or linked to a scaffold molecule.

A number of folate and folate analogs amenable to the present invention as ligands are described in U.S. Pat. Nos. 2,816,110; 5,552,545; 6,335,434 and 7,128,893, contents of which are herein incorporated in their entireties by reference.

As used herein, the terms “PK modulating ligand” and “PK modulator” refers to molecules which can modulate the pharmacokinetics of oligonucleotides described herein. Some exemplary PK modulator include, but are not limited to, lipophilic molecules, bile acids, sterols, phospholipid analogues, peptides, protein binding agents, vitamins, fatty acids, phenoxazine, aspirin, naproxen, ibuprofen, suprofen, ketoprofen, (S)-(+)-pranoprofen, carprofen, PEGs, biotin, and transthyretia-binding ligands (e.g., tetraiidothyroacetic acid, 2, 4, 6-triiodophenol and flufenamic acid). Oligomeric compounds that comprise a number of phosphorothioate intersugar linkages are also known to bind to serum protein, thus short oligomeric compounds, e.g.

oligonucleotides of comprising from about 5 to 30 nucleotides (e.g., 5 to 25 nucleotides, preferably 5 to 20 nucleotides, e.g., 5, 6, 7, 8, 9, 10, 11, 12, 13, 14, 15, 16, 17, 18, 19, or 20 nucleotides), and that comprise a plurality of phosphorothioate linkages in the backbone are also amenable to the present invention as ligands (e.g. as PK modulating ligands). The PK modulating oligonucleotide can comprise at least 3, 4, 5, 6, 7, 8, 9, 10, 11, 12, 13, 14, 15 or more phosphorothioate and/or phosphorodithioate linkages. In some embodiments, all internucleoside linkages in PK modulating oligonucleotide are phosphorothioate and/or phosphorodithioates linkages. In addition, aptamers that bind serum components (e.g. serum proteins) are also amenable to the present invention as PK modulating ligands. Binding to serum components (e.g. serum proteins) can be predicted from albumin binding assays, scuh as those described in Oravcova, et al., Journal of Chromatography B (1996), 677:1-27.

When two or more ligands are present, the ligands can all have same properties, all have different properties or some ligands have the same properties while others have different properties. For example, a ligand can have targeting properties, have endosomolytic activity or have PK modulating properties. In a preferred embodiment, all the ligands have different properties.

In some embodiments of any one of the aspects, the ligand has a structure shown in any of Formula (IV)—(VII):

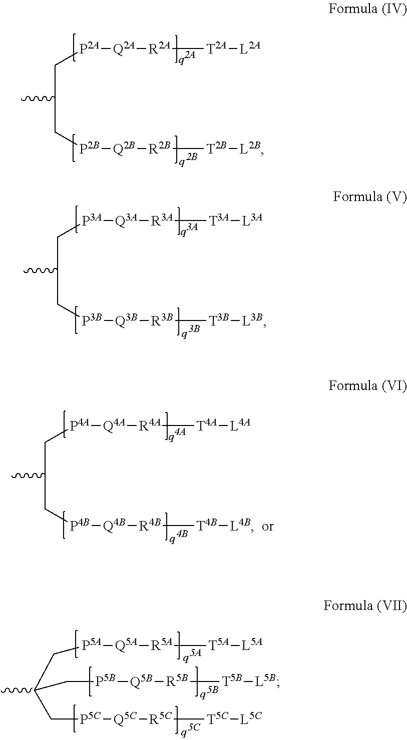

wherein:
q^{2A}, q^{2B}, q^{3A}, q^{3B}, q^{4A}, q^{4B}, q^{5A}, q^{5B} and q^{5C} represent independently for each occurrence 0-20 and wherein the repeating unit can be the same or different;
P^{2A}, P^{2B}, P^{3A}, P^{3B}, P^{4A}, P^{4B}, P^{5A}, P^{5B}, P^{5C}, T^{2A}, T^{2B}, T^{3A}, T^{3B}, T^{4A}, T^{4B}, T^{5A}, T^{5B}, T^{5C} are each independently for each occurrence absent, CO, NH, O, S, OC(O), NHC(O), CH₂, CH₂NH or CH₂O;
Q^{2A}, Q^{2B}, Q^{3A}, Q^{3B}, Q^{4A}, Q^{4B}, Q^{5A}, Q^{5B}, Q^{5C} are independently for each occurrence absent, alkylene, substituted alkylene wherein one or more methylenes can be interrupted or terminated by one or more of O, S, S(O), SO₂, N(RN), C(R′)═C(R″), C≡C or C(O);
R^{2A}, R^{2B}, R^{3A}, R^{3B}, R^{4A}, R^{4B}, R^{5A}, R^{5B}, R^{5C} are each independently for each occurrence absent, NH, O, S, CH₂, C(O)O, C(O)NH, NHCH(R^{a})C(O), —C(O)—CH(R^{a})—NH—, CO, CH═N—O,

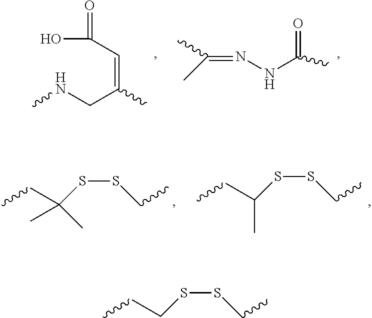

or heterocyclyl;
L^{2A}, L^{2B}, L^{3A}, L^{3B}, L^{4A}, L^{4B}, L^{5A}, L^{5B} and L^{5C} represent the ligand; i.e. each independently for each occurrence a monosaccharide (such as GalNAc), disaccharide, trisaccharide, tetrasaccharide, oligosaccharide, or polysaccharide; and
R^{a} is H or amino acid side chain.

In some embodiments of any one of the aspects, the ligand is of Formula (VII):

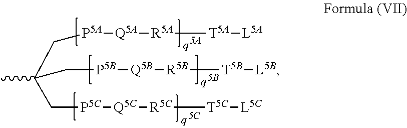

wherein L^{5A}, L^{5B} and L^{5C} represent a monosaccharide, such as GalNAc derivative.

Exemplary ligands include, but are not limited to, the following:

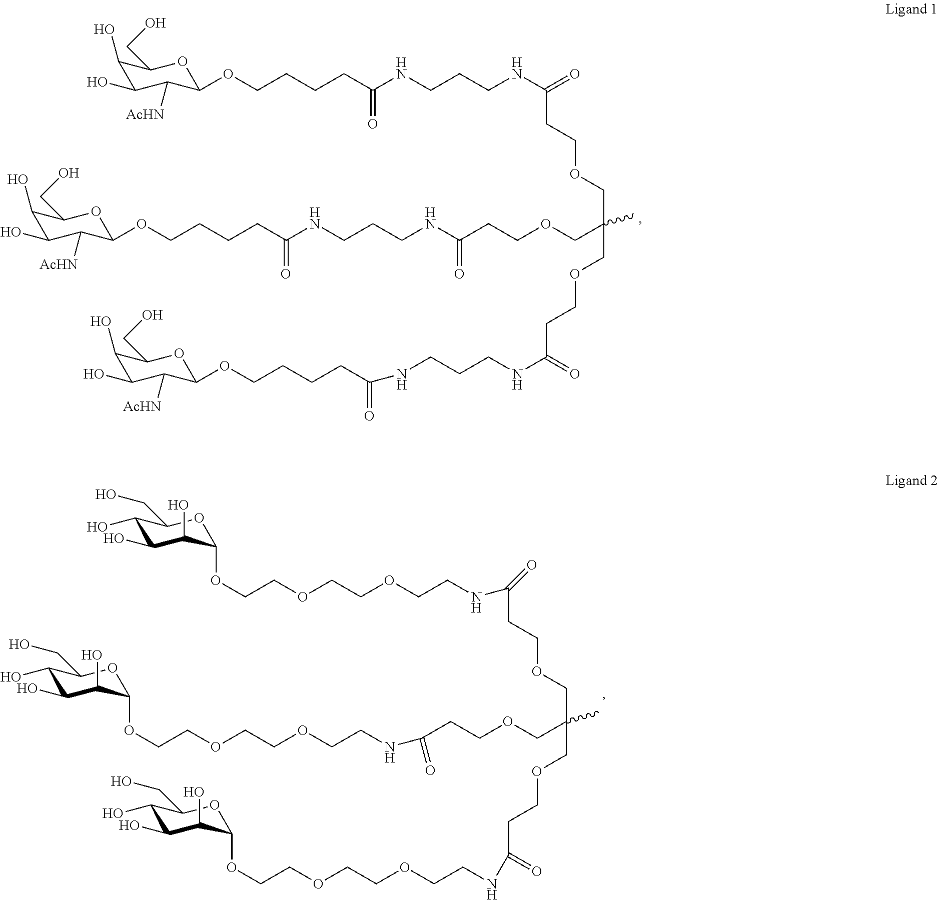

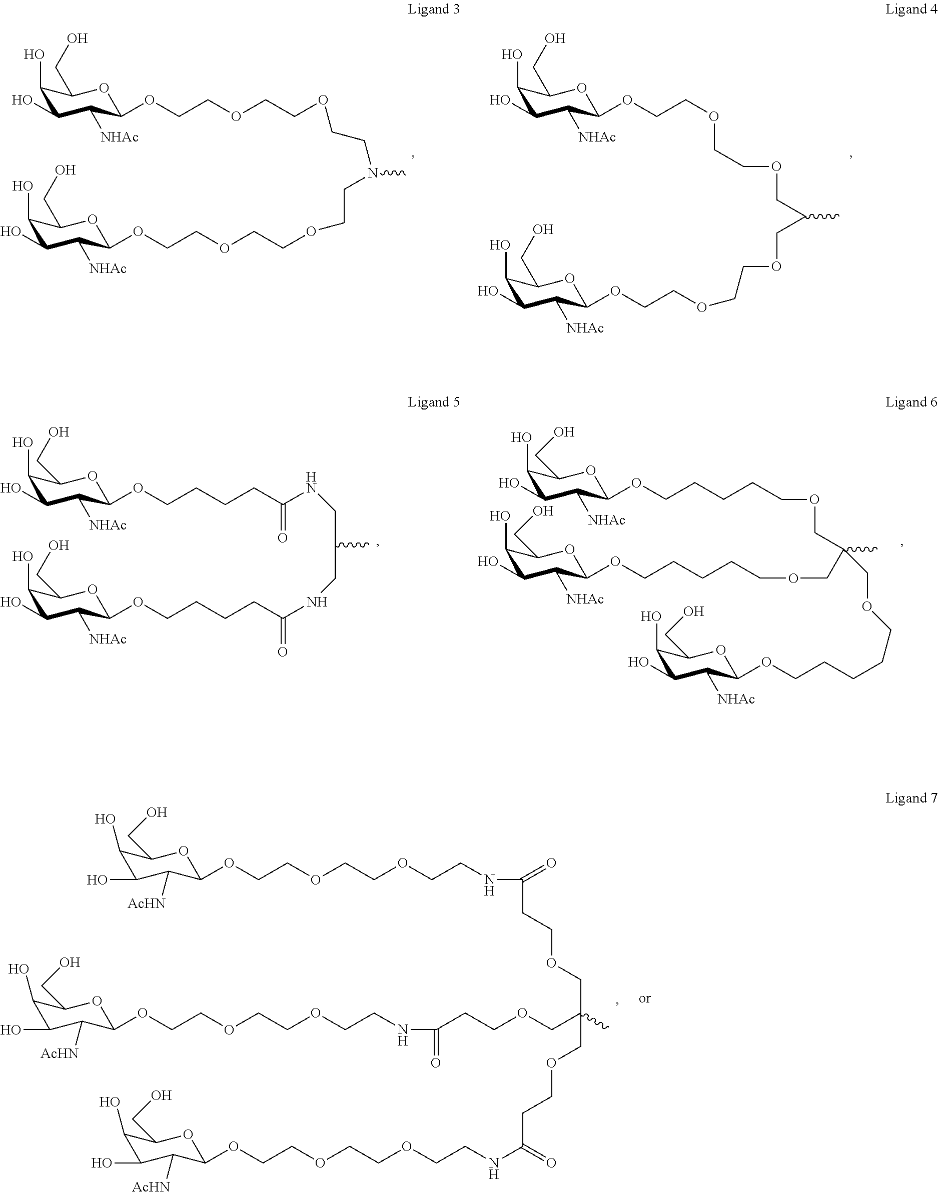

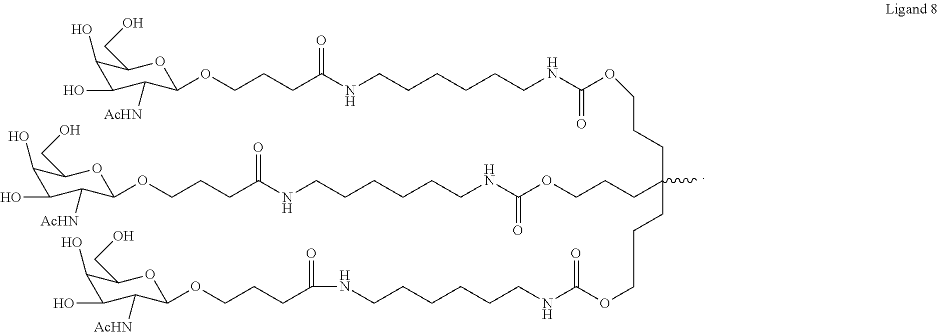 [SMILES: CC(=O)NC1C(OCCCC(=O)NCCCCCCNC(=O)OCCCC(C)(CCCOC(=O)NCCCCCCNC(=O)CCCOC2OC(CO)C(O)C(O)C2NC(C)=O)CCCOC(=O)NCCCCCCNC(=O)CCCOC2OC(CO)C(O)C(O)C2NC(C)=O)OC(CO)C(O)C1O]

In some embodiments of any one of the aspects described herein, the ligand is a ligand described in U.S. Pat. No. 5,994,517 or U.S. Pat. No. 6,906,182, content of each of which is incorporated herein by reference in its entirety.

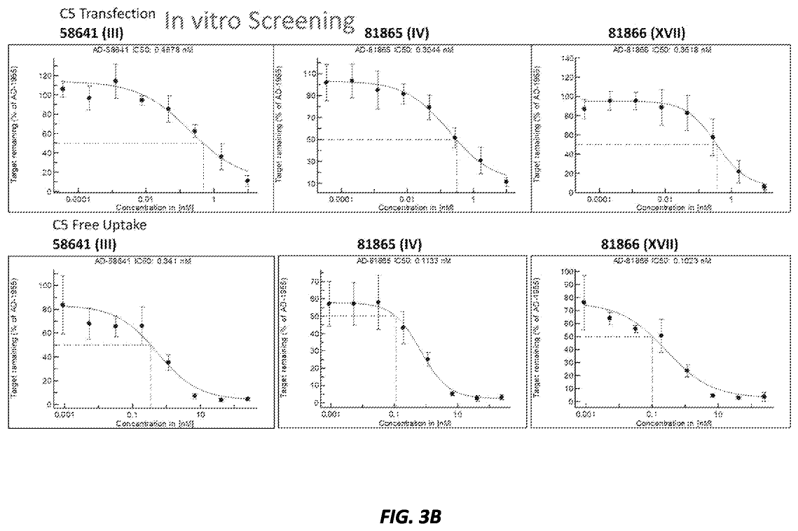

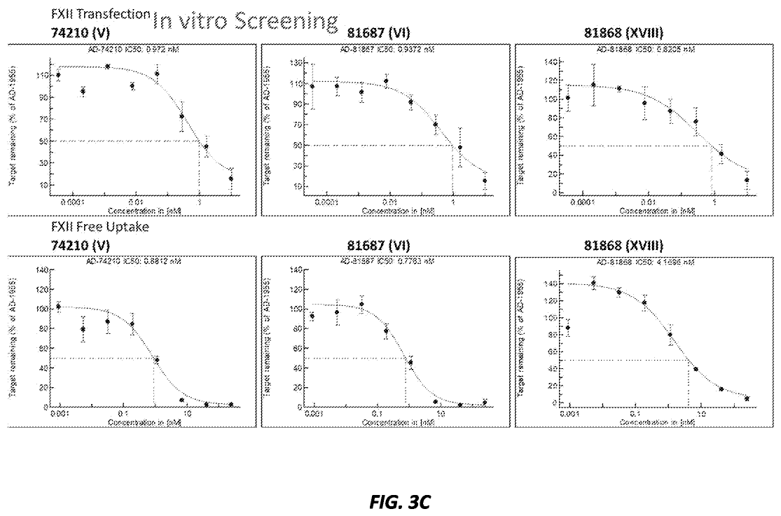

In some embodiments, the ligand can be a tri-antennary ligand described in FIG. 3 of U.S. Pat. No. 6,906,182. For example, the ligand is selected from the following tri-antennary ligands:

Tri-antennary

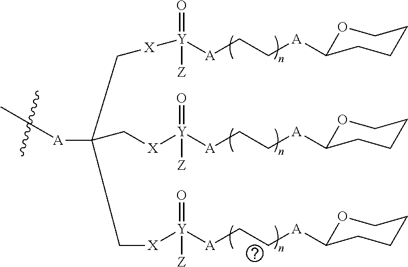

tris((heteroatom)methyl)-[heteroatom]methane

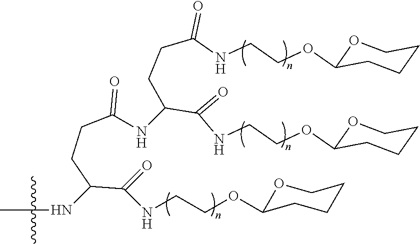

digfutamyl*

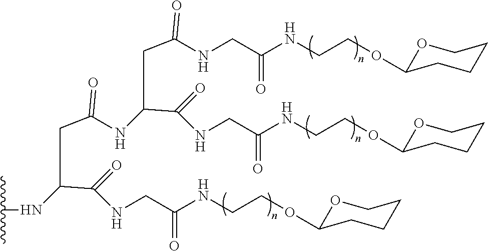

diasparatyl

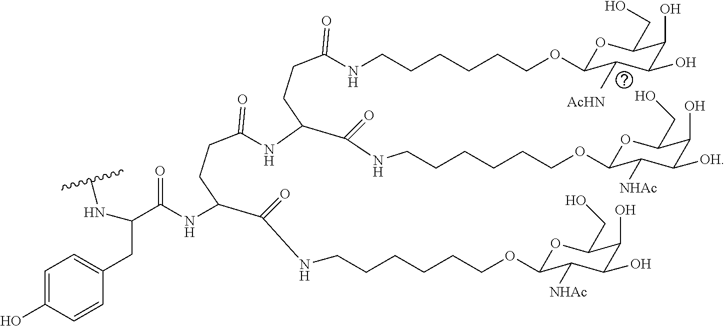 [SMILES: *NC(Cc1ccc(O)cc1)C(=O)NC(CCC(=O)NC(CCC(=O)NCCCCCCOC1CC(O)C(O)C(CO)O1)C(=O)NCCCCCCOC1OC(CO)C(O)C(O)C1NC(C)=O)C(=O)NCCCCCCOC1OC(CO)C(O)C(O)C1NC(C)=O ; "AcHN"]

X = NH, O, S
Y = P or S
Z = NH-alkyl, NH₂, O⁻, S⁻
A = NH, CH₂, O, S
n = 2 to 17.2-carbon units
Carbohydrate =

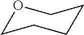

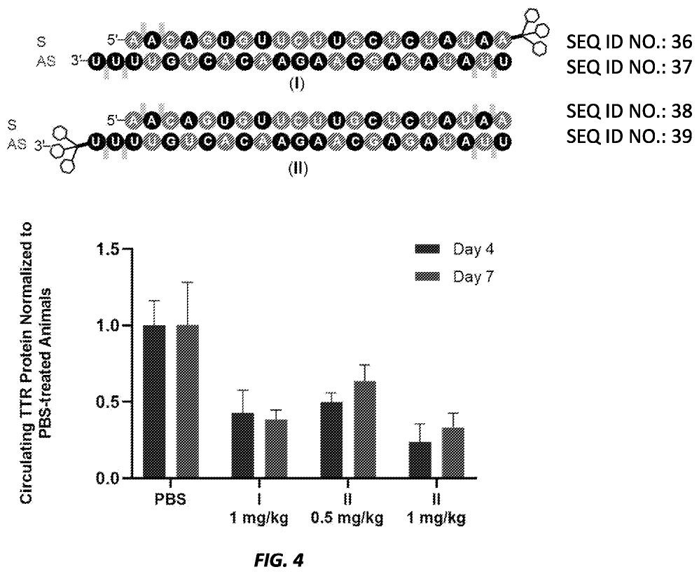

In some embodiments, the ligand can be a ligand described, e.g., in FIGS. 4A and 4B of US2021/0123048, contents of which are incorporated herein by reference in their entireties. In some embodiments of any one of the aspects described herein, the ligand can be

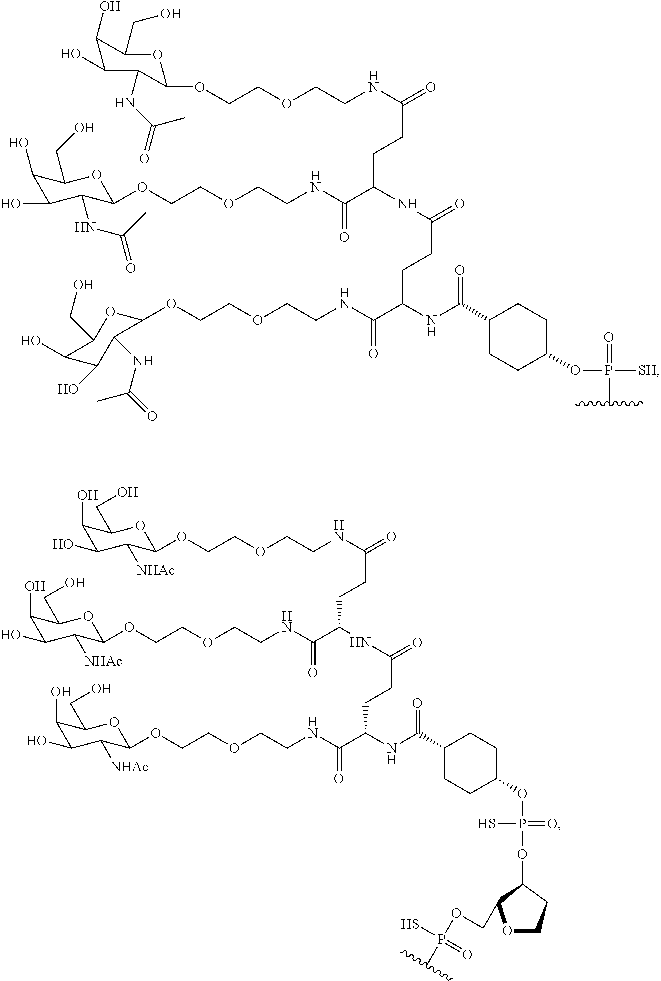

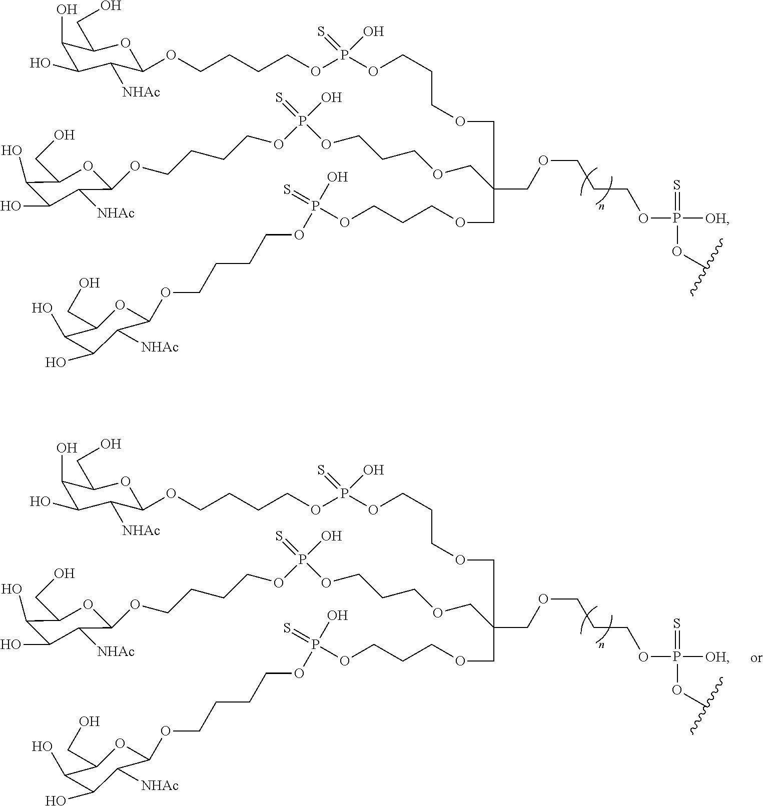

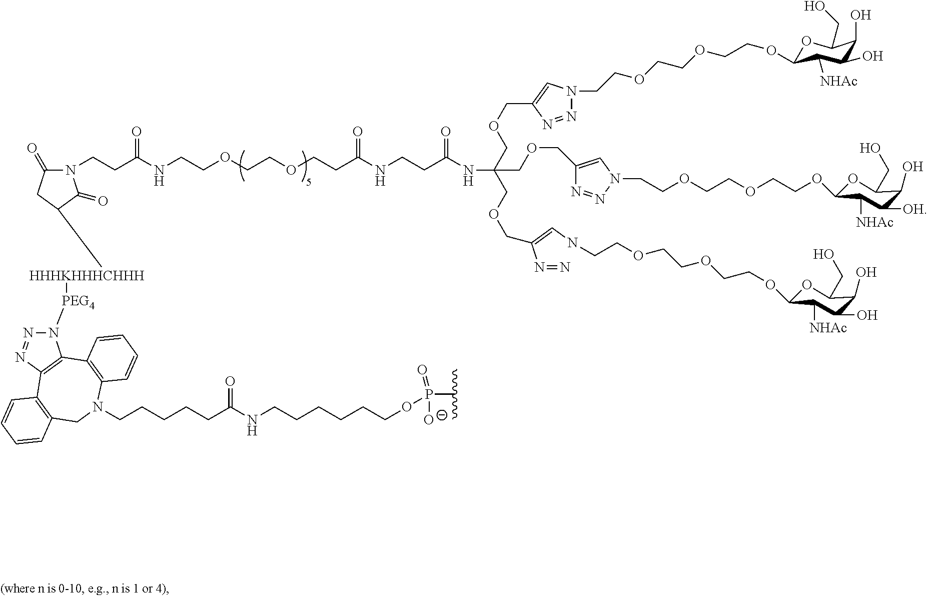

It is noted that when more than one ligands are present, they can be same or different. Accordingly, in some embodiments of any one of the aspects described herein, all ligands are same. In some other embodiments of any one of the aspects described herein, ligands are different.

Some exemplary ligands include, but are not limited to, peptides, centyrins, antibodies, antibody fragments, T-cell targeting ligands, B-cell targeting ligands, cancer cell targeting ligands (DUPA, folate, RGD), spleen targeting functionalities, lung targeting functionalitie, bone marrow targeting functionalities, antiCD-4 antobodies, antiCD-117 antibodies, phage Display peptides, cell permeation peptides (CPPs), itegrin ligands, multianionic ligands, multicationic ligands, carbohydrates (GalNAc, mannose, mannose-6 phosphate, fucose, glucose, monovalent and multivalent), kidney targeting ligands, blood-brain barrier (BBB) penetration ligands, lipids and amino acids (L-amino acids, D-amino acids, β-amino acids).

In some embodiments, the ligand comprises a lipophilic group. For example, the ligand can be a C₆-30aliphatic group or a C₁₀₋₃₀ aliphatic group. In some embodiments, the ligand is a C₁₀₋₃₀alkyl, C₁₀₋₃₀alkenyl or C₁₀₋₃₀alkynyl group. For example, the ligand is a straight-chain or branched hexyl, octyl, decyl, dodecyl, tetradecyl, hexadecyl, octadecyl, icosyl, docosyl, or tetracosyl group. In some embodiments, the ligand is a straight-chain hexyl, octyl, decyl, dodecyl, tetradecyl, hexadecyl, octadecyl, icosyl, docosyl, or tetracosyl group. For example, the ligand is a straight-chain hexyl, octyl, decyl, dodecyl, hexadecyl, octadecyl, icosyl, or docosyl group. For example, the ligand is a straight-chain hexadecyl group. In another example, the ligand is a straight-chain docosyl group.

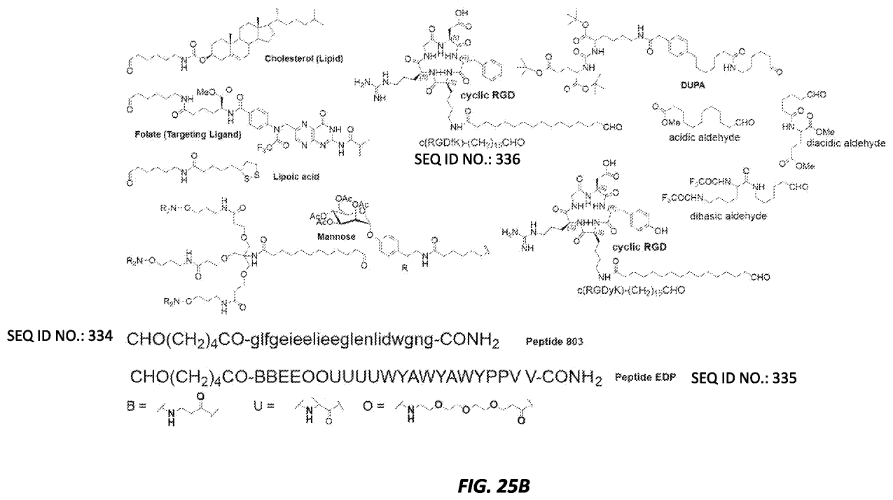

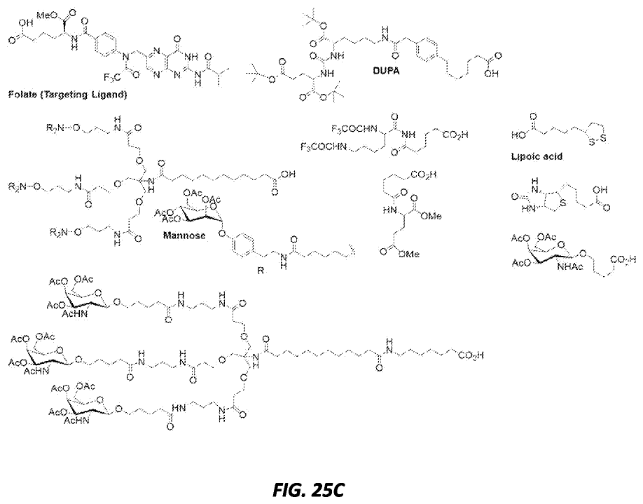

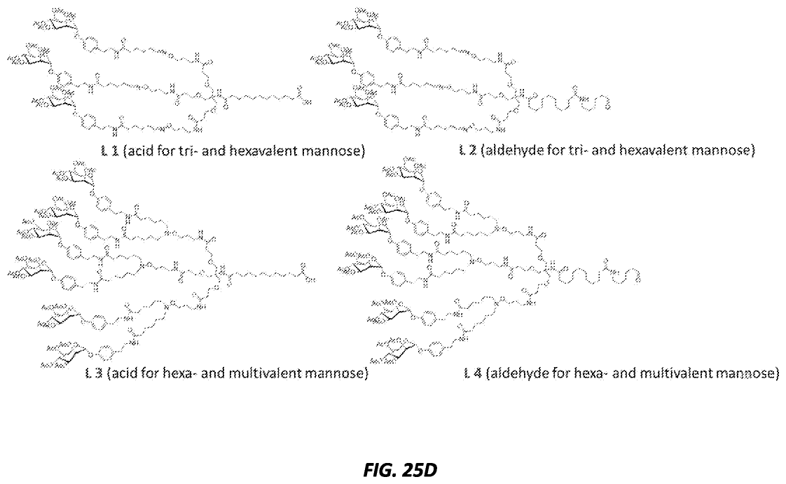

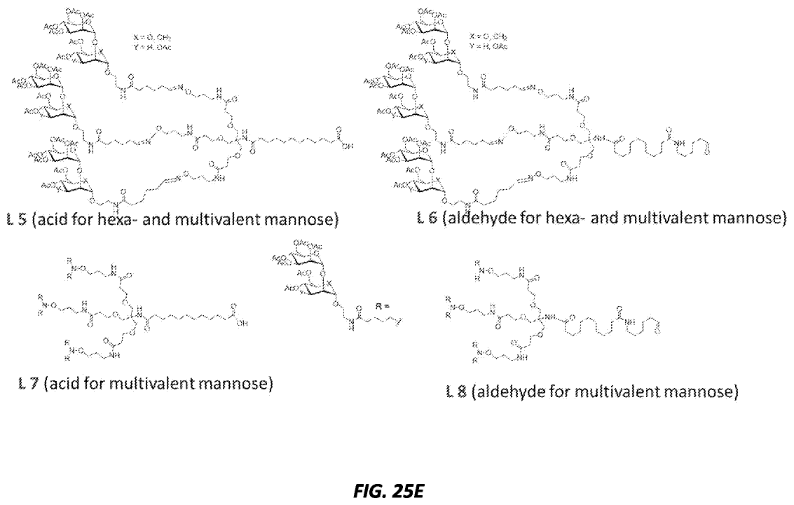

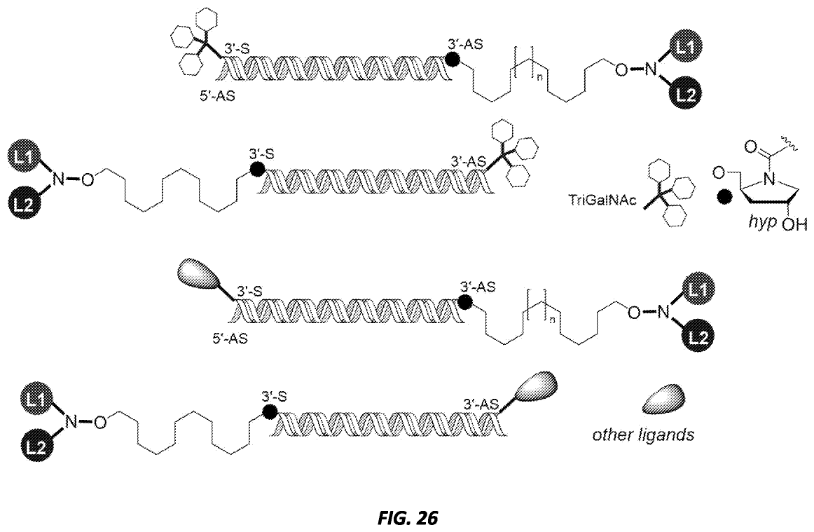

In some embodiments of any one of the aspects described herein, the ligand is selected from the group consisting of ligands shown in FIGS. 25A-25D.

### Linkers

Embodiments of the various aspects described herein include a linker. As used herein, the term “linker” means an organic moiety that connects two parts of a compound. Linkers typically comprise a direct bond or an atom such as oxygen or sulfur, a unit such as NR¹, C(O), C(O) O, C(O)NR¹, SO, SO₂, SO₂NH or a chain of atoms, such as substituted or unsubstituted alkyl, substituted or unsubstituted alkenyl, substituted or unsubstituted alkynyl, arylalkyl, arylalkenyl, arylalkynyl, heteroarylalkyl, heteroarylalkenyl, heteroarylalkynyl, heterocyclylalkyl, heterocyclylalkenyl, heterocyclylalkynyl, aryl, heteroaryl, heterocyclyl, cycloalkyl, cycloalkenyl, alkylarylalkyl, alkylarylalkenyl, alkylarylalkynyl, alkenylarylalkyl, alkenylarylalkenyl, alkenylarylalkynyl, alkynylarylalkyl, alkynylarylalkenyl, alkynylarylalkynyl, alkylheteroarylalkyl, alkylheteroarylalkenyl, alkylheteroarylalkynyl, alkenylheteroarylalkyl, alkenylheteroarylalkenyl, alkenylheteroarylalkynyl, alkynylheteroarylalkyl, alkynylheteroarylalkenyl, alkynylheteroarylalkynyl, alkylheterocyclylalkyl, alkylheterocyclylalkenyl, alkenylheterocyclylalkyl, alkenylheterocyclylalkenyl, alkylhererocyclylalkynyl, alkenylheterocyclylalkynyl, alkynylheterocyclylalkyl, alkynylheterocyclylalkenyl, alkynylheterocyclylalkynyl, alkylaryl, alkenylaryl, alkynylaryl, alkylheteroaryl, alkenylheteroaryl, alkynylhereroaryl, where one or more methylenes can be interrupted or terminated by O, S, S(O), SO₂, N(R^{LL}) 2, C(O), cleavable linking group, substituted or unsubstituted aryl, substituted or unsubstituted heteroaryl, substituted or unsubstituted heterocyclic; where R^{LL} is hydrogen, acyl, aliphatic or substituted aliphatic.

In some embodiments, the linker is a cleavable linker. Cleavable linkers are those that rely on processes inside a target cell to liberate the two parts the linker is holding together, as reduction in the cytoplasm, exposure to acidic conditions in a lysosome or endosome, or cleavage by specific enzymes (e.g. proteases) within the cell. As such, cleavable linkers allow the two parts to be released in their original form after internalization and processing inside a target cell. Cleavable linkers include, but are not limited to, those whose bonds can be cleaved by enzymes (e.g., peptide linkers); reducing conditions (e.g., disulfide linkers); or acidic conditions (e.g., hydrazones and carbonates).

Generally, the cleavable linker comprises at least one cleavable linking group. A cleavable linking group is one which is sufficiently stable outside the cell, but which upon entry into a target cell is cleaved to release the two parts the linker is holding together. In a preferred embodiment, the cleavable linking group is cleaved at least 10 times or more, preferably at least 100 times faster in the target cell or under a first reference condition (which can, e.g., be selected to mimic or represent intracellular conditions) than in the blood or serum of a subject, or under a second reference condition (which can, e.g., be selected to mimic or represent conditions found in the blood or serum).

Cleavable linking groups are susceptible to cleavage agents, e.g., pH, redox potential or the presence of degradative molecules. Generally, cleavage agents are more prevalent or found at higher levels or activities inside cells than in serum or blood. Examples of such degradative agents include: redox agents which are selected for particular substrates or which have no substrate specificity, including, e.g., oxidative or reductive enzymes or reductive agents such as mercaptans, present in cells, that can degrade a redox cleavable linking group by reduction; esterases; endosomes or agents that can create an acidic environment, e.g., those that result in a pH of five or lower; enzymes that can hydrolyze or degrade an acid cleavable linking group by acting as a general acid, peptidases (which can be substrate specific), and phosphatases.

A cleavable linkage group, such as a disulfide bond can be susceptible to pH. The pH of human serum is 7.4, while the average intracellular pH is slightly lower, ranging from about 7.1-7.3. Endosomes have a more acidic pH, in the range of 5.5-6.0, and lysosomes have an even more acidic pH at around 5.0. Some linkers will have a cleavable linking group that is cleaved at a preferred pH, thereby releasing the cationic lipid from the ligand inside the cell, or into the desired compartment of the cell.

A linker can include a cleavable linking group that is cleavable by a particular enzyme. The type of cleavable linking group incorporated into a linker can depend on the cell to be targeted. For example, liver targeting ligands can be linked to the cationic lipids through a linker that includes an ester group. Liver cells are rich in esterases, and therefore the linker will be cleaved more efficiently in liver cells than in cell types that are not esterase-rich. Other cell-types rich in esterases include cells of the lung, renal cortex, and testis. Linkers that contain peptide bonds can be used when targeting cell types rich in peptidases, such as liver cells and synoviocytes.

In general, the suitability of a candidate cleavable linking group can be evaluated by testing the ability of a degradative agent (or condition) to cleave the candidate linking group. It will also be desirable to also test the candidate cleavable linking group for the ability to resist cleavage in the blood or when in contact with other non-target tissue. Thus one can determine the relative susceptibility to cleavage between a first and a second condition, where the first is selected to be indicative of cleavage in a target cell and the second is selected to be indicative of cleavage in other tissues or biological fluids, e.g., blood or serum. The evaluations can be carried out in cell free systems, in cells, in cell culture, in organ or tissue culture, or in whole animals. It may be useful to make initial evaluations in cell-free or culture conditions and to confirm by further evaluations in whole animals. In preferred embodiments, useful candidate compounds are cleaved at least 2, 4, 10 or 100 times faster in the cell (or under in vitro conditions selected to mimic intracellular conditions) as compared to blood or serum (or under in vitro conditions selected to mimic extracellular conditions).

One class of cleavable linking groups is redox cleavable linking groups, which may be used according to the present invention that are cleaved upon reduction or oxidation. An example of reductively cleavable linking group is a disulfide linking group (—S—S—).

Phosphate-based cleavable linking groups, which may be used in the linkers according to the present invention, are cleaved by agents that degrade or hydrolyze the phosphate group. An example of an agent that cleaves phosphate groups in cells are enzymes such as phosphatases in cells. Examples of phosphate-based linking groups are —O—P(O)(OR^{k})—O—, —O—P(S)(OR^{k})—O—, —O—P(S)(SR^{k})—O—, —S—P(O)(OR^{k})—O—, —O—P(O)(OR)—S—, —S—P(O)(OR^{k})—S—, —O—P(S)(OR)—S—, —S—P(S)(OR^{k})—O—, —O—P(O)(R^{k})—O—, —O—P(S)(R^{k})—O—, —S—P(O)(R^{k})—O—, —S—P(S)(R^{k})—O—, —S—P(O)(R^{k})—S—, —O—P(S)(R^{k})—S—, wherein R^{k} at each occurrence can be, independently, hydrogen, C₁₋₂₀alkyl, C₁₋₂₀haloalkyl, C₆₋₁₀aryl, C₇₋₁₂aralkyl. Preferred embodiments are —O—P(O)(OH)—O—, —O—P(S)(OH)—O—, —O—P(S)(SH)—O—, —S—P(O)(OH)—O—, —O—P(O)(OH)—S—, —S—P(O)(OH)—S—, —O—P(S)(OH)—S—, —S—P(S)(OH)—O—, —O—P(O)(H)—O—, —O—P(S)(H)—O—, —S—P(O)(H)—O—, —S—P(S)(H)—O—, —S—P(O)(H)—S—, —O—P(S)(H)—S—. A preferred embodiment is —O—P(O)(OH)—O—. These candidates can be evaluated using methods analogous to those described above.

Acid cleavable linking groups, which may be used in the linkers according to the present invention, are linking groups that are cleaved under acidic conditions. In preferred embodiments acid cleavable linking groups are cleaved in an acidic environment with a pH of about 6.5 or lower (e.g., about 6.0, 5.5, 5.0, or lower), or by agents such as enzymes that can act as a general acid. In a cell, specific low pH organelles, such as endosomes and lysosomes can provide a cleaving environment for acid cleavable linking groups. Examples of acid cleavable linking groups include but are not limited to hydrazones, esters, and esters of amino acids. Acid cleavable groups can have the general formula —C═NN—, C(O) O, or —OC(O). A preferred embodiment is when the carbon attached to the oxygen of the ester (the alkoxy group) is an aryl group, substituted alkyl group, or tertiary alkyl group such as dimethyl pentyl or t-butyl. These candidates can be evaluated using methods analogous to those described above.

Ester-based cleavable linking groups, which may be used in the linkers according to the present invention, are cleaved by enzymes such as esterases and amidases in cells. Examples of ester-based cleavable linking groups include but are not limited to esters of alkylene, alkenylene and alkynylene groups. Ester cleavable linking groups have the general formula —C(O)O—, or —OC(O)—. These candidates can be evaluated using methods analogous to those described above.

Peptide-based cleavable linking groups, which may be used according to the present invention, are cleaved by enzymes such as peptidases and proteases in cells. Peptide-based cleavable linking groups are peptide bonds formed between amino acids to yield oligopeptides (e.g., dipeptides, tripeptides etc.) and polypeptides. Peptide-based cleavable groups do not include the amide group (—C(O)NH—). The amide group can be formed between any alkylene, alkenylene or alkynylene. A peptide bond is a special type of amide bond formed between amino acids to yield peptides and proteins. The peptide based cleavage group is generally limited to the peptide bond (i.e., the amide bond) formed between amino acids yielding peptides and proteins and does not include the entire amide functional group. Peptide-based cleavable linking groups have the general formula-NHCHRAC(O)NHCHRBC(O)—, where R^{A} and R^{B} are the R groups of the two adjacent amino acids.

In some embodiments of any one of the aspects described herein, the linker is a hydrophobic linker. For example, the linker comprises aliphatic, cycloaliphatic, and/or aromatic moieties. In some embodiments, the linker is a hydrophilic linker. For example, the linker comprises polyethylene glycol, e.g., the linker is —(CH₂CH₂O)_{w}—, where w is an integer. In some embodiments, w is an integer between 1 and 1000. For example, w is an integer between 2 and 500, e.g., w is 5, 10, 15, 20, 25, 30, 35, 40, 50, 100, 150, 200, 250, 300, 350, 400 or 500.

### Oligonucleotide Modifications

In some embodiments of any one of the aspects, the oligonucleotide can comprise one or more, e.g., 1, 2, 3, 4, 5, 6, 7, 8 or more modified internucleoside linkages. For example, the oligonucleotide can comprise 1, 2, 3, 4, 5 or 6 modified internucleoside linkages. For example, the oligonucleotide comprises 1, 2, 3 or 4 modified internucleoside linkages. In some embodiments, the oligonucleotide comprises at least two modified internucleoside linkages between the first five nucleotides counting from the 5′-end of the oligonucleotide and further comprises at least two modified internucleoside linkages between the first five nucleotides counting from the 3′-end of the oligonucleotide. For example, the oligonucleotide comprises modified internucleoside linkages between nucleotides 1 and 2, and between nucleotides 2 and 3, counting from 5′-end of the oligonucleotide, and between nucleotides 1 and 2, and between nucleotides 2 and 3, counting from 3′-end of the oligonucleotide.

In some embodiments of any one of the aspects, the oligonucleotide comprises one or more, e.g., 1, 2, 3, 4, 5, 6, 7, 8 or more phosphorothioate internucleoside linkages. For example, the oligonucleotide comprises 1, 2, 3, 4, 5 or 6 phosphorothioate internucleoside linkages. For example, the oligonucleotide comprises 1, 2, 3 or 4 phosphorothioate internucleoside linkages. In some embodiments, the oligonucleotide comprises at least two phosphorothioate internucleoside linkages between the first five nucleotides counting from the 5′-end of the oligonucleotide and further comprises at least two phosphorothioate internucleoside linkages between the first five nucleotides counting from the 3′-end of the oligonucleotide. For example, the oligonucleotide comprises modified internucleoside linkages between nucleotides 1 and 2, and between nucleotides 2 and 3, counting from 5′-end of the oligonucleotide, and between nucleotides 1 and 2, and between nucleotides 2 and 3, counting from 3′-end of the oligonucleotide.

In some embodiments of any one of the aspects described herein, the oligonucleotide further comprises, i.e., in addition to a nucleotiside of Formula (II), a nucleoside with a modified sugar. By a “modified sugar” is meant a sugar or moiety other than 2′-deoxy (i.e, 2′-H) or 2′-OH ribose sugar. Some exemplary nucleotides comprising a modified sugar are 2′-F ribose, 2′-OMe ribose, 2′-0,4′-C-methylene ribose (locked nucleic acid, LNA), anhydrohexitol (1,5-anhydrohexitol nucleic acid, HNA), cyclohexene (Cyclohexene nucleic acid, CeNA), 2′-methoxyethyl ribose, 2′-O-allyl ribose, 2′-C-allyl ribose, 2′-O—N-methylacetamido (2′-O-NMA) ribose, a 2′-O-dimethylaminoethoxyethyl (2′-O-DMAEOE) ribose, 2′-O-aminopropyl (2′-O-AP) ribose, 2′-F arabinose (2′-ara-F), threose (Threose nucleic acid, TNA), and 2,3-dihydroxylpropyl (glycol nucleic acid, GNA). It is noted that the nucleoside with the modified sugar can be present at any position of the oligonucleotide.

In some embodiments, the oligonucleotide further comprises at least one, e.g., 1, 2, 3, 4, 5, 6, 7, 8, 9, 10 or more 2′-fluoro (2′-F) nucleotides. For example, the oligonucleotide can comprise 1, 2, 3, 4, 5, 6, 7, 8, 9 or 10 2′—F nucleotides. It is noted that the 2′-F nucleotides can be present at any position of the oligonucleotide.

In some embodiments, the oligonucleotide comprises, e.g., solely comprises nucleosides of Formula (II), and 2′-F nucleosides.

In some embodiments, the oligonucleotide further comprises at least one, e.g., 1, 2, 3, 4, 5, 6, 7, 8, 9, 10 or more 2′-OMe nucleotides. For example, the oligonucleotide can comprise 1, 2, 3, 4, 5, 6, 7, 8, 9 or 10 2′—OMe nucleotides. It is noted that the 2′-OMe nucleotides can be present at any position of the oligonucleotide.

In some embodiments, the oligonucleotide comprises, e.g., solely comprises solely comprises nucleosides of Formula (II), and 2′-OMe nucleosides. In some other embodiments, the oligonucleotide comprises, e.g., solely comprises nucleosides of Formula (II), 2′-OMe nucleosides and 2′-F nucleosides.

In some embodiments, the oligonucleotide further comprises at least one, e.g., 1, 2, 3, 4, 5, 6, 7, 8, 9, 10 or more 2′-deoxy, e.g., 2′-H nucleotides. For example, the oligonucleotide can comprise 1, 2, 3, 4, 5, 6, 7, 8, 9 or 10 of 2′-deoxy, e.g., 2′-H nucleotides. It is noted that the 2′-deoxy, e.g., 2′-H nucleotides can be present at any position of the oligonucleotide. For example, the oligonucleotide can comprise a 2′-deoxy, e.g., 2′-H nucleotide at 1, 2, 3, 4, 5 or 6 of positions 2, 5, 7, 12, 14 and 16, counting from 5′-end of the oligonucleotide. In some embodiments, the oligonucleotide comprises a 2′-deoxy nucleotide at positions 5 and 7, counting from 5′-end of the oligonucleotide.

In some embodiments, the oligonucleotide comprises, e.g., solely comprises nucleosides of Formula (II), and 2′-deoxy (2′-H) nucleotides. In some embodiments, the oligonucleotide comprises, e.g., solely comprises nucleosides of Formula (II), 2′-OMe nucleosides, and 2′-deoxy (2′-H) nucleotides. In some embodiments, the oligonucleotide comprises, e.g., solely comprises nucleosides of Formula (II), 2′-F nucleosides and 2′-deoxy (2′-H) nucleotides. In some embodiments, the oligonucleotide comprises, e.g., solely comprises nucleosides of Formula (II), 2′-OMe nucleosides, 2′-F nucleosides and 2′-deoxy (2′-H) nucleotides.

In some embodiments of any one of the aspects described herein, the oligonucleotide further comprises, i.e., in addition to a nucleoside of Formula (II), a non-natural nucleobase. In some embodiments, the oligonucleotide can comprise one or more, e.g., 1, 2, 3, 4, 5, 6, 7, 8, 9, 10 or more nucleotides comprising an independently selected non-natural nucleobase. When present, a nucleotide comprising a non-natural nucleobase can be present anywhere in the oligonucleotide.

In some embodiments, the oligonucleotide further comprises a solid support linked thereto.

The oligonucleotides described herein can range from few nucleotides (e.g., 2, 3, 4, 5, 6, 7, 8, 9 or 10 nucleotides) in length to hundreds of nucleotides in length. For example, the oligonucleotide can be from 5 nucleotides to 100 nucleotides in length. In some embodiments, the oligonucleotide is from 10 nucleotides to 50 nucleotides in length. For example, the oligonucleotide is between 15 and 35, more generally between 18 and 25, yet more generally between 19 and 24, and most generally between 19 and 21 base pairs in length. In some embodiments, longer oligonucleotides of between 25 and 30 nucleotides in length are preferred. In some embodiments, shorter oligonucleotides of between 10 and 15 nucleotides in length are preferred. In another embodiment, the oligonucleotide is at least 21 nucleotides in length.

In some embodiments of any one of the aspects, the oligonucleotide described herein can comprise a thermally destabilizing modification. For example, the oligonucleotide can comprise at least one thermally destabilizing modification of the duplex within the first 9 nucleotide positions, counting from the 5′-end of the oligonucleotide. In some embodiments, the thermally destabilizing modification is located at position 2, 3, 4, 5, 6, 7, 8 or 9, counting from the 5′-end of the antisense strand. In some embodiments, thermally destabilizing modification is located in positions 2-9, or preferably positions 4-8, counting from the 5′-end of the oligonucleotide. In some further embodiments, the thermally destabilizing modification is located at position 5, 6, 7 or 8, counting from the 5′-end of the oligonucleotide. In still some further embodiments, the thermally destabilizing modification is located at position 7, counting from the 5′-end of the oligonucleotide.

In some embodiments of any one of the aspects described herein, the oligonucleotide can comprise one or more stabilizing modifications. For example, the oligonucleotide can comprise at least two (e.g., two, three, four, five, six, seven, eight, nine, ten or more) stabilizing modifications.

In some embodiments, the oligonucleotide comprises at least two (e.g., two, three, four, five, six, seven, eight, nine, ten or more) stabilizing modifications. Without limitations, a stabilizing modification in the oligonucleotide can be present at any positions. In some embodiments, the oligonucleotide comprises stabilizing modifications at positions 2, 6, 8, 9, 14 and 16, counting from the 5′-end. In some other embodiments, the oligonucleotide comprises stabilizing modifications at positions 2, 6, 14 and 16, counting from the 5′-end. In still some other embodiments, the oligonucleotide comprises stabilizing modifications at positions 2, 14 and 16, counting from the 5′-end. In some embodiments, the oligonucleotide comprises stabilizing modifications at positions 7, 10 and 11, counting from the 5′-end. In some other embodiments, the oligonucleotide comprises stabilizing modifications at positions 7, 9, 10 and 11, counting from the 5′-end.

In some embodiments, the oligonucleotide comprises at least one stabilizing modification adjacent to a destabilizing modification. For example, the stabilizing modification can be the nucleotide at the 5′-end or the 3′-end of the destabilizing modification, i.e., at position-1 or +1 from the position of the destabilizing modification. In some embodiments, the oligonucleotide comprises a stabilizing modification at each of the 5′-end and the 3′-end of the destabilizing modification, i.e., positions-1 and +1 from the position of the destabilizing modification. In some embodiments, the oligonucleotide comprises at least two stabilizing modifications at the 3′-end of a destabilizing modification.

### Methods of Inhibiting Expression of a Target Gene

In another aspect, the disclosure provides methods of using the oligonucleotides and dsNRAs described herein. For example, provided herein is a method for inhibiting the expression of a target gene in a cell. The method comprising administering to said cell a dsRNA molecule or oligonucleotide described herein, where the antisense strand or the oligonucleotide comprises a nucleotide sequence substantially complementary to a nucleotide sequence of the target gene. It is noted that administering to the cell can be in vitro or in vivo. Accordingly, the present disclosure further relates to a use of a dsRNA molecule or oligonucleotide described herein for inhibiting expression of a target gene in a target cell in vitro. When the cell is in vivo, the method comprises administering to a subject in an amount sufficient to inhibit expression of the target gene a double-stranded RNA or oligonucleotide described herein, where the antisense strand or the oligonucleotide comprises a nucleotide sequence substantially complementary to a nucleotide sequence of a target gene. In some embodiments, the subject has or has been diagnosed with a disease or disorder.

The target gene can be any desired RNA molecule, including, but not limited to, messenger RNA (mRNA), transfer RNA (tRNA), ribosomal RNA (rRNA) and microRNA (miRNA). In some preferred embodiments, the target gene is a mRNA. In some embodiments, the target nucleic acid comprises a nucleotide sequence associated with a disease or disorder.

In some embodiments, the target gene is selected from the group consisting of Factor VII, Eg5, PCSK9, TPX2, apoB, SAA, TTR, RSV, PDGF beta gene, Erb-B gene, Src gene, CRK gene, GRB2 gene, RAS gene, MEKK gene, JNK gene, RAF gene, Erk1/2 gene, PCNA (p21) gene, MYB gene, JUN gene, FOS gene, BCL-2 gene, hepcidin, Activated Protein C, Cyclin D gene, VEGF gene, EGFR gene, Cyclin A gene, Cyclin E gene, WNT-1 gene, beta-catenin gene, c-MET gene, PKC gene, NFKB gene, STAT3 gene, survivin gene, Her2/Neu gene, topoisomerase I gene, topoisomerase II alpha gene, mutations in the p73 gene, mutations in the p21 (WAF1/CIP1) gene, mutations in the p27 (KIP1) gene, mutations in the PPMID gene, mutations in the RAS gene, mutations in the caveolin I gene, mutations in the MIB I gene, mutations in the MTAI gene, mutations in the M68 gene, mutations in tumor suppressor genes, and mutations in the p53 tumor suppressor gene.

### Cells

The disclosure also provides a cell comprising a dsRNA or oligonucleotide described herein. As used herein, the term “cell” refers to a single cell as well as to a population of (i.e., more than one) cells.

### Kits

A dsRNA or oligonucleotide described herein can be provided in a kit, e.g., as a component of a kit. For example, the kit includes (a) a dsRNA or oligonucleotide described herein, and optionally (b) informational material. The informational material can be descriptive, instructional, marketing, or other material that relates to the methods described herein and/or the use of a dsRNA or oligonucleotide described herein for the methods described herein. The informational material of the kits is not limited in its form. In some embodiments, the informational material can include information about production of the dsRNAs or oligonucleotides, their molecular weight, concentration, date of expiration, batch, or production site information, and so forth. In some embodiments, the informational material relates to using dsRNA or oligonucleotide to treat, prevent, or diagnosis of disorders and conditions.

In some embodiments, the informational material can include instructions to administer the dsRNA or oligonucleotide in a suitable manner to perform the methods described herein, e.g., in a suitable dose, dosage form, or mode of administration (e.g., a dose, dosage form, or mode of administration described herein). In another embodiment, the informational material can include instructions to administer the dsRNA or oligonucleotide to a suitable subject, e.g., a human, e.g., a human having, or at risk for, a disorder or condition needing treatment.

The informational material of the kits is not limited in its form. In many cases, the informational material, e.g., instructions, is provided in print but can also be in other formats, such as computer readable material.

Components of the kit, e.g., the dsRNA or oligonucleotide can be provided in any form, e.g., liquid, dried or lyophilized form. It is preferred that the dsRNA or oligonucleotide be substantially pure and/or sterile. When the dsRNA or oligonucleotide is provided in a liquid solution, the liquid solution preferably is an aqueous solution, with a sterile aqueous solution being preferred. When the dsRNA or oligonucleotide is provided as a dried form, reconstitution generally is by the addition of a suitable solvent. The solvent, e.g., sterile water or buffer, can optionally be provided in the kit.

The kit can include one or more containers for the components of the kit. In some embodiments, the kit contains separate containers, dividers, or compartments for the different components of the kit. For example, the dsRNA or oligonucleotide can be contained in a bottle, vial, or syringe, and the informational material can be contained association with the container. In other embodiments, the separate elements of the kit are contained within a single, undivided container. For example, the dsRNA or oligonucleotide is contained in a bottle, vial or syringe that has attached thereto the informational material in the form of a label. In some embodiments, the kit includes a plurality (e.g., a pack) of individual containers, each containing one or more-unit dosage forms of the dsRNA or oligonucleotide. For example, the kit includes a plurality of syringes, ampules, foil packets, or blister packs, each containing a single unit dose of the dsRNA or oligonucleotide. The containers of the kits can be airtight, waterproof (e.g., impermeable to changes in moisture or evaporation), and/or light-tight.

The kit optionally includes a device suitable for administration of the dsRNA or oligonucleotide, e.g., a syringe, inhalant, dropper (e.g., eye dropper), swab (e.g., a cotton swab or wooden swab), or any such delivery device. In some embodiments, the device is an implantable device that dispenses metered doses of the dsRNA or oligonucleotide. The disclosure also features a method of providing a kit, e.g., by combining components described herein.

In some embodiments, the kit can further comprise additional components and/or reagents for practicing the methods described herein using the dsRNA or oligonucleotide described herein.

### Compositions

The dsRNA or oligonucleotide described herein can be formulated in compositions. For example, the dsRNA or oligonucleotide described herein can be formulated into pharmaceutical compositions for therapeutic use. Accordingly, in another aspect, the invention provides a pharmaceutical composition comprising a dsRNA or oligonucleotide described herein. Pharmaceutically acceptable compositions comprise a therapeutically-effective amount of one or more of the dsRNA or oligonucleotide described herein, taken alone, or formulated together with one or more pharmaceutically acceptable carriers (additives), excipient and/or diluents.

The pharmaceutical compositions can be specially formulated for administration in solid or liquid form, including those adapted for the following: (1) oral administration, for example, drenches (aqueous or non-aqueous solutions or suspensions), tablets, e.g., those targeted for buccal, sublingual, and systemic absorption, boluses, powders, granules, pastes for application to the tongue; (2) parenteral administration, for example, by subcutaneous, intramuscular, intravenous or epidural injection as, for example, a sterile solution or suspension, or sustained-release formulation; (3) topical application, for example, as a cream, ointment, or a controlled-release patch or spray applied to the skin; (4) intravaginally or intrarectally, for example, as a pessary, cream or foam; (5) sublingually; (6) ocularly; (7) transdermally; or (8) nasally. Delivery using subcutaneous or intravenous methods can be particularly advantageous.

The phrase “therapeutically-effective amount” as used herein means that amount of a compound, material, or composition comprising a conjugate described herein which is effective for producing some desired therapeutic effect in at least a sub-population of cells in an animal at a reasonable benefit/risk ratio applicable to any medical treatment.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The phrase “pharmaceutically acceptable carrier” as used herein means a pharmaceutically-acceptable material, composition, or vehicle, such as a liquid or solid filler, diluent, excipient, manufacturing aid (e.g., lubricant, talc magnesium, calcium or zinc stearate, or steric acid), or solvent encapsulating material, involved in carrying or transporting the subject compound from one organ, or portion of the body, to another organ, or portion of the body. Each carrier must be “acceptable” in the sense of being compatible with the other ingredients of the formulation and not injurious to the patient. Some examples of materials which can serve as pharmaceutically acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) lubricating agents, such as magnesium state, sodium lauryl sulfate and talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18)Ringer's solution; (19) ethyl alcohol; (20) pH buffered solutions; (21) polyesters, polycarbonates and/or polyanhydrides; (22) bulking agents, such as polypeptides and amino acids (23) serum component, such as serum albumin, HDL and LDL; and (22) other non-toxic compatible substances employed in pharmaceutical formulations.

As used herein, a “pharmaceutically acceptable carrier” is intended to include any and all solvents, dispersion media, coatings, antibacterial and antifungal agents, isotonic and absorption delaying agents, and the like, compatible with pharmaceutical administration. The use of such media and agents for pharmaceutically active substances is well known in the art. Except insofar as any conventional media or agent is incompatible with the active compound, use thereof in the compositions is contemplated. Supplementary active compounds can also be incorporated into the compositions. Pharmaceutical carriers include sterile aqueous solutions or dispersions and sterile powders for the extemporaneous preparation of sterile injectable solutions or dispersion. The use of such media and agents for pharmaceutically active substances is known in the art.

The formulations can conveniently be presented in unit dosage form and can be prepared by any methods well known in the art of pharmacy. The amount of active ingredient which can be combined with a carrier material to produce a single dosage form will vary depending upon the host being treated, the particular mode of administration. The amount of active ingredient which can be combined with a carrier material to produce a single dosage form will generally be that amount of the compound which produces a therapeutic effect. Generally, out of one hundred percent, this amount will range from about 0.1 percent to about ninety-nine percent of active ingredient, preferably from about 5 percent to about 70 percent, most preferably from about 10 percent to about 30 percent.

Pharmaceutical compositions for use with the methods described herein can be formulated in a conventional manner using one or more physiologically acceptable carriers or excipients. For example, a dsRNA or oligonucleotide described herein can be formulated for administration by, for example, by aerosol, intravenous, oral, or topical route. The compositions can be formulated for intralesional, intratumoral, intraperitoneal, subcutaneous, intramuscular, or intravenous injection; infusion; liposome-mediated delivery; topical, intrathecal, gingival pocket, per rectum, intrabronchial, nasal, transmucosal, intestinal, oral, ocular, or otic delivery.

Techniques and formulations generally can be found in Remington's Pharmaceutical Sciences, Meade Publishing Co., Easton, PA. For systemic administration, injection is preferred, including intramuscular, intravenous, intraperitoneal, and subcutaneous. For injection, a dsRNA described herein can be formulated in liquid solutions, preferably in physiologically compatible buffers such as Hank's solution or Ringer's solution. In addition, the dsRNA can be formulated in solid form and redissolved or suspended immediately prior to use. Lyophilized forms are also included.

For oral administration, the pharmaceutical composition can take the form of, for example, tablets or capsules prepared by conventional means with pharmaceutically acceptable excipients such as binding agents (e.g., pregelatinized maize starch, polyvinylpyrrolidone or hydroxypropyl methylcellulose); fillers (e.g., lactose, microcrystalline cellulose or calcium hydrogen phosphate); lubricants (e.g., magnesium stearate, talc or silica); disintegrants (e.g., potato starch or sodium starch glycolate); or wetting agents (e.g., sodium lauryl sulphate). The tablets can be coated by methods well known in the art. Liquid preparations for oral administration can take the form of, for example, solutions, syrups, or suspensions, or they may be presented as a dry product for constitution with water or other suitable vehicle before use. Such liquid preparations can be prepared by conventional means with pharmaceutically acceptable additives such as suspending agents (e.g., sorbitol syrup, cellulose derivatives, or hydrogenated edible fats); emulsifying agents (e.g., lecithin or acacia); non-aqueous vehicles (e.g., pharmaceutically acceptable oils, oily esters, ethyl alcohol or fractionated vegetable oils); and preservatives (e.g., methyl or propyl-p-hydroxybenzoates or sorbic acid). The preparations can also contain buffer salts, flavoring, coloring, and sweetening agents as appropriate.

Preparations for oral administration can be suitably formulated to give controlled release of the active compound. For buccal administration the compositions can take the form of tablets or lozenges formulated in conventional manner. For administration by inhalation, the compounds for use as described herein are conveniently delivered in the form of an aerosol spray presentation from pressurized packs or a nebulizer, with the use of a suitable propellant, e.g., dichlorodifluoromethane, trichlorofluoromethane, dichlorotetrafluoroethane, carbon dioxide or other suitable gas. In the case of a pressurized aerosol the dosage unit can be determined by providing a valve to deliver a metered amount. Capsules and cartridges of e.g., gelatin for use in an inhaler or insufflator can be formulated containing a powder mix of the compound and a suitable powder base such as lactose or starch.

The dsRNA or oligonucleotide can be formulated for parenteral administration by injection, e.g., by bolus injection or continuous infusion. Formulations for injection can be presented in unit dosage form, e.g., in ampoules or in multi-dose containers, with an added preservative. The compositions can take such forms as suspensions, solutions, or emulsions in oily or aqueous vehicles, and can contain formulatory agents such as suspending, stabilizing and/or dispersing agents. Alternatively, the active ingredient can be in powder form for constitution with a suitable vehicle, e.g., sterile pyrogen-free water, before use.

In addition to the formulations described previously, the dsRNA or oligonucleotide can also be formulated as a depot preparation. Such long-acting formulations can be administered by implantation (for example subcutaneously or intramuscularly) or by intramuscular injection. Thus, for example, dsRNAs or oligonucleotides can be formulated with suitable polymeric or hydrophobic materials (for example as an emulsion in an acceptable oil) or ion exchange resins, or as sparingly soluble derivatives, for example, as a sparingly soluble salt.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration bile salts and fusidic acid derivatives. In addition, detergents can be used to facilitate permeation. Transmucosal administration can be through nasal sprays or using suppositories. For topical administration, dsRNA can be formulated into ointments, salves, gels, or creams as generally known in the art. A wash solution can be used locally to treat an injury or inflammation to accelerate healing.

The compositions can, if desired, be presented in a pack or dispenser device which can contain one or more-unit dosage forms containing the active ingredient. The pack can for example comprise metal or plastic foil, such as a blister pack. The pack or dispenser device can be accompanied by instructions for administration.

### Liposomes and Lipid Formulations

The dsRNAs or oligonucleotides described herein can be formulated for delivery in a membranous molecular assembly, e.g., a liposome or a micelle. As used herein, the term “liposome” refers to a vesicle composed of amphiphilic lipids arranged in at least one bilayer, e.g., one bilayer or a plurality of bilayers. Liposomes include unilamellar and multilamellar vesicles that have a membrane formed from a lipophilic material and an aqueous interior. The aqueous portion contains the dsRNA or oligonucleotide. The lipophilic material isolates the aqueous interior from an aqueous exterior, which typically does not include the dsRNA or oligonucleotide, although in some examples, it may. Liposomes are useful for the transfer and delivery of active ingredients to the site of action. Because the liposomal membrane is structurally similar to biological membranes, when liposomes are applied to a tissue, the liposomal bilayer fuses with bilayer of the cellular membranes. As the merging of the liposome and cell progresses, the internal aqueous contents that include a dsRNA or oligonucleotide described herein are delivered into the cell. In some cases, the liposomes are also specifically targeted, e.g., to direct the conjugate to particular cell types.

A liposome containing a dsRNA or oligonucleotide described herein can be prepared by a variety of methods. In one example, the lipid component of a liposome is dissolved in a detergent so that micelles are formed with the lipid component. For example, the lipid component can be an amphipathic cationic lipid or lipid conjugate. The detergent can have a high critical micelle concentration and may be nonionic. Exemplary detergents include cholate, CHAPS, octylglucoside, deoxycholate, and lauroyl sarcosine. The dsRNA is then added to the micelles that include the lipid component. After condensation, the detergent is removed, e.g., by dialysis, to yield a liposomal preparation.

If necessary, a carrier compound that assists in condensation can be added during the condensation reaction, e.g., by controlled addition. For example, the carrier compound can be a polymer other than a nucleic acid (e.g., spermine or spermidine). pH can also be adjusted to favor condensation.

Further description of methods for producing stable polynucleotide or oligonucleotide delivery vehicles, which incorporate a polynucleotide/cationic lipid complex as structural components of the delivery vehicle, are described in, e.g., WO 96/37194. Liposome formation can also include one or more aspects of exemplary methods described in Felgner, P. L. et al., Proc. Natl. Acad. Sci., USA 8:7413-7417, 1987; U.S. Pat. Nos. 4,897,355; 5,171,678; Bangham, et al. M. Mol. Biol. 23:238, 1965; Olson, et al. Biochim. Biophys. Acta 557:9, 1979; Szoka, et al. Proc. Natl. Acad. Sci. 75:4194, 1978; Mayhew, et al. Biochim. Biophys. Acta 775:169, 1984; Kim, et al. Biochim. Biophys. Acta 728:339, 1983; and Fukunaga, et al. Endocrinol. 115:757, 1984, which are incorporated by reference in their entirety. Commonly used techniques for preparing lipid aggregates of appropriate size for use as delivery vehicles include sonication and freeze-thaw plus extrusion (see, e.g., Mayer, et al. Biochim. Biophys. Acta 858:161, 1986, which is incorporated by reference in its entirety). Microfluidization can be used when consistently small (50 to 200 nm) and relatively uniform aggregates are desired (Mayhew, et al. Biochim. Biophys. Acta 775:169, 1984, which is incorporated by reference in its entirety).

Liposomes that are pH-sensitive or negatively-charged entrap nucleic acid molecules rather than complex with them. Since both the nucleic acid molecules and the lipid are similarly charged, repulsion rather than complex formation occurs. Nevertheless, some nucleic acid molecules are entrapped within the aqueous interior of these liposomes. pH-sensitive liposomes have been used to deliver DNA encoding the thymidine kinase gene to cell monolayers in culture. Expression of the exogenous gene was detected in the target cells (Zhou et al., Journal of Controlled Release, 19, (1992)₂₆₉-274, which is incorporated by reference in its entirety).

One major type of liposomal composition includes phospholipids other than naturally-derived phosphatidylcholine. Neutral liposome compositions, for example, can be formed from dimyristoyl phosphatidylcholine (DMPC) or dipalmitoyl phosphatidylcholine (DPPC). Anionic liposome compositions generally are formed from dimyristoyl phosphatidylglycerol, while anionic fusogenic liposomes are formed primarily from dioleoyl phosphatidylethanolamine (DOPE). Another type of liposomal composition is formed from phosphatidylcholine (PC) such as, for example, soybean PC, and egg PC. Another type is formed from mixtures of phospholipid and/or phosphatidylcholine and/or cholesterol.

Examples of other methods to introduce liposomes into cells in vitro and include U.S. Pat. Nos. 5,283,185; 5,171,678; WO 94/00569; WO 93/24640; WO 91/16024; Felgner, J. Biol. Chem. 269:2550, 1994; Nabel, Proc. Natl. Acad. Sci. 90:11307, 1993; Nabel, Human Gene Ther. 3:649, 1992; Gershon, Biochem. 32:7143, 1993; and Strauss EMBO J. 11:417, 1992.

In some embodiments, cationic liposomes are used. Cationic liposomes possess the advantage of being able to fuse to the cell membrane.

Further advantages of liposomes include, but are not limited to, liposomes obtained from natural phospholipids are biocompatible and biodegradable; liposomes can incorporate a wide range of water and lipid soluble drugs; liposomes can protect encapsulated dsRNAs in their internal compartments from metabolism and degradation (Rosoff, in “Pharmaceutical Dosage Forms,” Lieberman, Rieger and Banker (Eds.), 1988, volume 1, p. 245). Important considerations in the preparation of liposome formulations are the lipid surface charge, vesicle size and the aqueous volume of the liposomes.

A positively charged synthetic cationic lipid, N-[1-(2,3-dioleyloxy) propyl]-N,N,N-trimethylammonium chloride (DOTMA) can be used to form small liposomes that interact spontaneously with nucleic acid to form lipid-nucleic acid complexes which are capable of fusing with the negatively charged lipids of the cell membranes of tissue culture cells.

A DOTMA analogue, 1,2-bis(oleoyloxy)-3-(trimethylammonium) propane (DOTAP) can be used in combination with a phospholipid to form DNA-complexing vesicles. Lipofectin™ Bethesda Research Laboratories, Gaithersburg, Md.) is an effective agent for the delivery of highly anionic nucleic acids into living tissue culture cells that comprise positively charged DOTMA liposomes which interact spontaneously with negatively charged polynucleotides to form complexes. When enough positively charged liposomes are used, the net charge on the resulting complexes is also positive. Positively charged complexes prepared in this way spontaneously attach to negatively charged cell surfaces, fuse with the plasma membrane, and efficiently deliver functional nucleic acids into, for example, tissue culture cells. Another commercially available cationic lipid, 1,2-bis(oleoyloxy)-3,3-(trimethylammonium) propane (“DOTAP”) (Boehringer Mannheim, Indianapolis, Indiana) differs from DOTMA in that the oleoyl moieties are linked by ester, rather than ether linkages.

Other reported cationic lipid compounds include those that have been conjugated to a variety of moieties including, for example, carboxyspermine which has been conjugated to one of two types of lipids and includes compounds such as 5-carboxyspermylglycine dioctaoleoylamide (“DOGS”) (Transfectam™, Promega, Madison, Wisconsin) and dipalmitoylphosphatidylethanolamine 5-carboxyspermyl-amide (“DPPES”) (see, e.g., U.S. Pat. No. 5,171,678).

Another cationic lipid conjugate includes derivatization of the lipid with cholesterol (“DC-Chol”) which has been formulated into liposomes in combination with DOPE (See, Gao, X. and Huang, L., Biochim. Biophys. Res. Commun. 179:280, 1991). Lipopolylysine, made by conjugating polylysine to DOPE, has been reported to be effective for transfection in the presence of serum (Zhou, X. et al., Biochim. Biophys. Acta 1065:8, 1991, which is incorporated by reference in its entirety). For certain cell lines, these liposomes containing conjugated cationic lipids, are said to exhibit lower toxicity and provide more efficient transfection than the DOTMA-containing compositions. Other commercially available cationic lipid products include DMRIE and DMRIE-HP (Vical, La Jolla, California) and Lipofectamine (DOSPA) (Life Technology, Inc., Gaithersburg, Maryland). Other cationic lipids suitable for the delivery of oligonucleotides are described in WO 98/39359 and WO 96/37194.

Liposomal formulations are particularly suited for topical administration. Liposomes present several advantages over other formulations. Such advantages include reduced side effects related to high systemic absorption of the administered drug, increased accumulation of the administered drug at the desired target, and the ability to administer the dsRNA, into the skin. In some implementations, liposomes are used for delivering dsRNA to epidermal cells and also to enhance the penetration of dsRNA into dermal tissues, e.g., into skin. For example, the liposomes can be applied topically. Topical delivery of drugs formulated as liposomes to the skin has been documented (see, e.g., Weiner et al., Journal of Drug Targeting, 1992, vol. 2,405-410 and du Plessis et al., Antiviral Research, 18, 1992, 259-265; Mannino, R. J. and Fould-Fogerite, S., Biotechniques 6:682-690, 1988; Itani, T. et al. Gene 56:267-276. 1987; Nicolau, C. et al. Meth. Enz. 149:157-176, 1987; Straubinger, R. M. and Papahadjopoulos, D. Meth. Enz. 101:512-527, 1983; Wang, C. Y. and Huang, L., Proc. Natl. Acad. Sci. USA 84:7851-7855, 1987, which are incorporated by reference in their entirety).

Non-ionic liposomal systems have also been examined to determine their utility in the delivery of drugs to the skin, in particular systems comprising non-ionic surfactant and cholesterol. Non-ionic liposomal formulations comprising Novasome I (glyceryl dilaurate/cholesterol/polyoxyethylene-10-stearyl ether) and Novasome II (glyceryl distearate/cholesterol/polyoxyethylene-10-stearyl ether) were used to deliver a drug into the dermis of mouse skin.

Liposomes that include a dsRNA or oligonucleotide described herein can be made highly deformable. Such deformability can enable the liposomes to penetrate through pore that are smaller than the average radius of the liposome. For example, transfersomes are a type of deformable liposomes. Transfersomes can be made by adding surface edge activators, usually surfactants, to a standard liposomal composition. Transfersomes that include dsRNA or oligonucleotide can be delivered, for example, subcutaneously by infection. In order to cross intact mammalian skin, lipid vesicles must pass through a series of fine pores, each with a diameter less than 50 nm, under the influence of a suitable transdermal gradient. In addition, due to the lipid properties, these transfersomes can be self-optimizing (adaptive to the shape of pores, e.g., in the skin), self-repairing, and can frequently reach their targets without fragmenting, and often self-loading.

Other formulations amenable to the present invention are described in U.S. provisional application Ser. No. 61/018,616, filed Jan. 2, 2008; 61/018,611, filed Jan. 2, 2008; 61/039,748, filed Mar. 26, 2008; 61/047,087, filed Apr. 22, 2008, and 61/051,528, filed May 8, 2008. PCT application no PCT/US2007/080331, filed Oct. 3, 2007, also describes formulations that are amenable to the present invention.

Surfactants. Surfactants find wide application in formulations such as emulsions (including microemulsions) and liposomes (see above). A conjugate formulation can include a surfactant. In some embodiments, a conjugate described herein is formulated as an emulsion that includes a surfactant. The most common way of classifying and ranking the properties of the many different types of surfactants, both natural and synthetic, is by the use of the hydrophile/lipophile balance (HLB). The nature of the hydrophilic group provides the most useful means for categorizing the different surfactants used in formulations (Rieger, in “Pharmaceutical Dosage Forms,” Marcel Dekker, Inc., New York, NY, 1988, p. 285).

If the surfactant molecule is not ionized, it is classified as a nonionic surfactant. Nonionic surfactants find wide application in pharmaceutical products and are usable over a wide range of pH values. In general, their HLB values range from 2 to about 18 depending on their structure. Nonionic surfactants include nonionic esters such as ethylene glycol esters, propylene glycol esters, glyceryl esters, polyglyceryl esters, sorbitan esters, sucrose esters, and ethoxylated esters. Nonionic alkanolamides and ethers such as fatty alcohol ethoxylates, propoxylated alcohols, and ethoxylated/propoxylated block polymers are also included in this class. The polyoxyethylene surfactants are the most popular members of the nonionic surfactant class.

If the surfactant molecule carries a negative charge when it is dissolved or dispersed in water, the surfactant is classified as anionic. Anionic surfactants include carboxylates such as soaps, acyl lactylates, acyl amides of amino acids, esters of sulfuric acid such as alkyl sulfates and ethoxylated alkyl sulfates, sulfonates such as alkyl benzene sulfonates, acyl isethionates, acyl taurates and sulfosuccinates, and phosphates. The most important members of the anionic surfactant class are the alkyl sulfates and the soaps.

If the surfactant molecule carries a positive charge when it is dissolved or dispersed in water, the surfactant is classified as cationic. Cationic surfactants include quaternary ammonium salts and ethoxylated amines. The quaternary ammonium salts are the most used members of this class.

If the surfactant molecule has the ability to carry either a positive or negative charge, the surfactant is classified as amphoteric. Amphoteric surfactants include acrylic acid derivatives, substituted alkylamides, N-alkylbetaines and phosphatides.

The use of surfactants in drug products, formulations and in emulsions has been reviewed (Rieger, in “Pharmaceutical Dosage Forms,” Marcel Dekker, Inc., New York, NY, 1988, p. 285).

Micelles and other Membranous Formulations. Formulations comprising a conjugate described herein can be provided as a micellar formulation. “Micelles” are defined herein as a particular type of molecular assembly in which amphipathic molecules are arranged in a spherical structure such that all the hydrophobic portions of the molecules are directed inward, leaving the hydrophilic portions in contact with the surrounding aqueous phase. The converse arrangement exists if the environment is hydrophobic.

A mixed micellar formulation suitable for delivery through transdermal membranes may be prepared by mixing an aqueous solution of the dsRNA or oligonucleotide, an alkali metal C₈ to C₂₂ alkyl sulphate, and a micelle forming compounds. Exemplary micelle forming compounds include lecithin, hyaluronic acid, pharmaceutically acceptable salts of hyaluronic acid, glycolic acid, lactic acid, chamomile extract, cucumber extract, oleic acid, linoleic acid, linolenic acid, monoolein, monooleates, monolaurates, borage oil, evening of primrose oil, menthol, trihydroxy oxo cholanyl glycine and pharmaceutically acceptable salts thereof, glycerin, polyglycerin, lysine, polylysine, triolein, polyoxyethylene ethers and analogues thereof, polidocanol alkyl ethers and analogues thereof, chenodeoxycholate, deoxycholate, and mixtures thereof. The micelle forming compounds may be added at the same time or after addition of the alkali metal alkyl sulphate. Mixed micelles will form with substantially any kind of mixing of the ingredients but vigorous mixing in order to provide smaller size micelles.

In one method a first micellar composition is prepared which contains conjugate described herein and at least the alkali metal alkyl sulphate. The first micellar composition is then mixed with at least three micelle forming compounds to form a mixed micellar composition. In another method, the micellar composition is prepared by mixing conjugate described herein, the alkali metal alkyl sulphate and at least one of the micelle forming compounds, followed by addition of the remaining micelle forming compounds, with vigorous mixing.

Phenol and/or m-cresol may be added to the mixed micellar composition to stabilize the formulation and protect against bacterial growth. Alternatively, phenol and/or m-cresol may be added with the micelle forming ingredients. An isotonic agent such as glycerin may also be added after formation of the mixed micellar composition.

For delivery of the micellar formulation as a spray, the formulation can be put into an aerosol dispenser and the dispenser is charged with a propellant. The propellant, which is under pressure, is in liquid form in the dispenser. The ratios of the ingredients are adjusted so that the aqueous and propellant phases become one, i.e., there is one phase. If there are two phases, it is necessary to shake the dispenser prior to dispensing a portion of the contents, e.g., through a metered valve. The dispensed dose of pharmaceutical agent is propelled from the metered valve in a fine spray.

Propellants may include hydrogen-containing chlorofluorocarbons, hydrogen-containing fluorocarbons, dimethyl ether, and diethyl ether. In certain embodiments, HFA 134a (1,1,1,2 tetrafluoroethane) may be used.

The specific concentrations of the essential ingredients can be determined by relatively straightforward experimentation. For absorption through the oral cavities, it is often desirable to increase, e.g., at least double or triple, the dosage for through injection or administration through the gastrointestinal tract.

Particles. In some embodiments, conjugate described herein can be incorporated into a particle, e.g., a microparticle. Microparticles can be produced by spray-drying, but may also be produced by other methods including lyophilization, evaporation, fluid bed drying, vacuum drying, or a combination of these techniques.

Methods of preparing the formulations or compositions include the step of bringing into association an oligonucleotide and/or dsRNA with the carrier and, optionally, one or more accessory ingredients. In general, the formulations are prepared by uniformly and intimately bringing into association a compound of the present invention with liquid carriers, or finely divided solid carriers, or both, and then, if necessary, shaping the product.

In some cases, in order to prolong the effect of a drug, it is desirable to slow the absorption of the drug from subcutaneous or intramuscular injection. This may be accomplished by the use of a liquid suspension of crystalline or amorphous material having poor water solubility. The rate of absorption of the drug then depends upon its rate of dissolution which, in turn, may depend upon crystal size and crystalline form. Alternatively, delayed absorption of a parenterally-administered drug form is accomplished by dissolving or suspending the drug in an oil vehicle.

The oligonucleotide and/or dsRNA described herein may be formulated for administration in any convenient way for use in human or veterinary medicine, by analogy with other pharmaceuticals.

The oligonucleotide and/or dsRNA described herein or a pharmaceutical composition comprising an oligonucleotide and/or dsRNA described herein can be administered to a subject using different routes of delivery. A composition that includes an oligonucleotide and/or dsRNA described herein described herein can be delivered to a subject by a variety of routes. Exemplary routes include: intravenous, subcutaneous, topical, rectal, anal, vaginal, nasal, pulmonary, ocular.

The oligonucleotide and/or dsRNA described herein may be administered in a number of ways depending upon whether local or systemic treatment is desired and upon the area to be treated. Administration may be topical (including ophthalmic, vaginal, rectal, intranasal, transdermal), oral or parenteral. Parenteral administration includes intravenous drip, subcutaneous, intraperitoneal or intramuscular injection, or intrathecal or intraventricular administration.

The route and site of administration may be chosen to enhance targeting. For example, to target muscle cells, intramuscular injection into the muscles of interest would be a logical choice. Lung cells might be targeted by administering the oligonucleotide and/or dsRNA described herein in aerosol form. The vascular endothelial cells could be targeted by coating a balloon catheter with the oligonucleotide and/or dsRNA described herein and mechanically introducing the oligonucleotide and/or dsRNA described herein.

In one aspect, provided herein is a method of administering an oligonucleotide and/or dsRNA described herein, to a subject (e.g., a human subject). In another aspect, the present invention relates to an oligonucleotide and/or dsRNA described herein for use in inhibiting expression of a target gene in a subject. The method or the medical use includes administering a unit dose of the oligonucleotide and/or dsRNA described herein. In some embodiments, the unit dose is less than 10 mg per kg of bodyweight, or less than 10, 5, 2, 1, 0.5, 0.1, 0.05, 0.01, 0.005, 0.001, 0.0005, 0.0001, 0.00005 or 0.00001 mg per kg of bodyweight, and less than 200 nmole of RNA agent (e.g., about 4.4×10¹⁶ copies) per kg of bodyweight, or less than 1500, 750, 300, 150, 75, 15, 7.5, 1.5, 0.75, 0.15, 0.075, 0.015, 0.0075, 0.0015, 0.00075, 0.00015 nmole of oligonucleotide and/or dsRNA described herein per kg of bodyweight.

The defined amount can be an amount effective to treat or prevent a disease or disorder, e.g., a disease or disorder associated with the target gene. The unit dose, for example, can be administered by injection (e.g., intravenous, subcutaneous or intramuscular), an inhaled dose, or a topical application. In some embodiments dosages may be less than 10, 5, 2, 1, or 0.1 mg/kg of body weight.

In some embodiments, the unit dose is administered less frequently than once a day, e.g., less than every 2, 4, 8 or 30 days. In another embodiment, the unit dose is not administered with a frequency (e.g., not a regular frequency). For example, the unit dose may be administered a single time.

In some embodiments, the effective dose is administered with other traditional therapeutic modalities.

In some embodiments, a subject is administered an initial dose and one or more maintenance doses. The maintenance dose or doses can be the same or lower than the initial dose, e.g., one-half less of the initial dose. A maintenance regimen can include treating the subject with a dose or doses ranging from 0.01 μg to 15 mg/kg of body weight per day, e.g., 10, 1, 0.1, 0.01, 0.001, or 0.00001 mg per kg of bodyweight per day. The maintenance doses are, for example, administered no more than once every 2, 5, 10, or 30 days. Further, the treatment regimen may last for a period of time which will vary depending upon the nature of the particular disease, its severity and the overall condition of the patient. In certain embodiments the dosage may be delivered no more than once per day, e.g., no more than once per 24, 36, 48, or more hours, e.g., no more than once for every 5 or 8 days. Following treatment, the patient can be monitored for changes in his condition and for alleviation of the symptoms of the disease state. The dosage of the compound may either be increased in the event the patient does not respond significantly to current dosage levels, or the dose may be decreased if an alleviation of the symptoms of the disease state is observed, if the disease state has been ablated, or if undesired side-effects are observed.

The effective dose can be administered in a single dose or in two or more doses, as desired or considered appropriate under the specific circumstances. If desired to facilitate repeated or frequent infusions, implantation of a delivery device, e.g., a pump, semi-permanent stent (e.g., intravenous, intraperitoneal, intracisternal or intracapsular), or reservoir may be advisable.

In some embodiments, the composition includes a plurality of dsRNA or oligonucleotide species. In another embodiment, the dsRNA or oligonucleotide species has sequences that are non-overlapping and non-adjacent to another species with respect to a naturally occurring target sequence. In another embodiment, the plurality of dsRNA or oligonucleotide species is specific for different naturally occurring target genes. In another embodiment, the dsRNA molecule is allele specific.

The oligonucleotide and/or dsRNA described herein can be administered to mammals, particularly large mammals such as nonhuman primates or humans in a number of ways.

In some embodiments, the administration of the oligonucleotide and/or dsRNA composition described herein is parenteral, e.g., intravenous (e.g., as a bolus or as a diffusible infusion), intradermal, intraperitoneal, intramuscular, intrathecal, intraventricular, intracranial, subcutaneous, transmucosal, buccal, sublingual, endoscopic, rectal, oral, vaginal, topical, pulmonary, intranasal, urethral or ocular. Administration can be provided by the subject or by another person, e.g., a health care provider. The medication can be provided in measured doses or in a dispenser which delivers a metered dose.

In some embodiments, the administration of the oligonucleotide and/or dsRNA described herein is subcutaneous or intravenous administration.

### Oxygen Protecting Groups

Some embodiments of the various aspects described herein include an oxygen protecting group (also referred to as a hydroxyl protecting group herein). Oxygen protecting groups include, but are not limited to, —R^{OP1}, —N(R^{OP2})₂, —C(═O)SR^{OP1}, —C(═O)R^{OP1}, —CO₂R^{OP1}, —C(═O)N(R^{OP2})₂, —C(═NR^{OP2})R^{OP1}, —C(═NR^{OP2})OR^{OP1}, —C(═NR^{OP2}) N(R^{OP2})₂, —S(═O)R^{OP1}, —SO⁺₂R^{OP1}, —Si(R^{OP1})₃, —P(R^{OP3})₂, —P(R^{OP3})⁺₃X⁻, —P(OR^{OP3})₂, —P(OR^{OP3})⁺₃ X⁻, —P(═O)(R^{OP1})₂, —P(═O)(OR^{OP3})₂, and —P(═O)(N(R^{OP2})₂)₂; wherein each X⁻ is a counterion; each R^{OP1} is independently C₁₋₁₀ alkyl, C₁₋₁₀ perhaloalkyl, C₂₋₁₀ alkenyl, C₂₋₁₀ alkynyl, heteroC₁₋₁₀ alkyl, heteroC₂₋₁₀alkenyl, heteroC₂₋₁₀alkynyl, C₃₋₁₀ carbocyclyl, 3-14 membered heterocyclyl, C₆₋₁₄ aryl, or 5-14 membered heteroaryl, or two R^{OP1} groups are joined to form a 3-14 membered heterocyclyl or 5-14 membered heteroaryl ring; each R^{OP2} is hydrogen, OH, OR^{OP1}, —N(R^{OP3})₂, —CN, —C(═O)R^{OP1}, —C(═O)N(R^{OP3})₂, CO₂R^{OP1}, —SO₂R^{OP1}, —C(═NR^{OP3})OR^{OP1}, —C(—NR^{OP3}) N(R^{OP3})₂, —SO₂N(R^{OP3})₂, —SO₂R^{OP3}, —SO₂OR^{OP3}, —SOR^{OP1}, —C(═S) N(R^{OP3})₂, C(═O)SR^{OP3}, —C(═S) SR^{OP3}, —P(═O) (R^{OP1})₂, —P(═O)(OR^{OP3})₂, —P(═O)(N(R^{OP3})₂)₂, C₁₋₁₀ alkyl, C₁₋₁₀ perhaloalkyl, C₂₋₁₀ alkenyl, C₂₋₁₀ alkynyl, heteroC₁₋₁₀alkyl, heteroC₂₋₁₀alkenyl, heteroC₂₋₁₀alkynyl, C₃₋₁₀ carbocyclyl, 3-14 membered heterocyclyl, C₆₋₁₄ aryl, and 5-14 membered heteroaryl, or two R^{OP2} groups are joined to form a 3-14 membered heterocyclyl or 5-14 membered heteroaryl ring; and each R^{OP3} is independently hydrogen, C₁₋₁₀ alkyl, C₁₋₁₀ perhaloalkyl, C₂₋₁₀ alkenyl, C₂₋₁₀ alkynyl, heteroC₁₋₁₀ alkyl, heteroC₂₋₁₀ alkenyl, heteroC₂₋₁₀ alkynyl, C₃₋₁₀ carbocyclyl, 3-14 membered heterocyclyl, C₆₋₁₄ aryl, and 5-14 membered heteroaryl, or two R^{OP3} groups are joined to form a 3-14 membered heterocyclyl or 5-14 membered heteroaryl ring; and wherein each alkyl, alkenyl, alkynyl, carbocyclyl, heterocyclyl, aralkyl, aryl, and heteroaryl of R^{OP1}, R^{OP2} and R^{OP3} can be optionally substituted with 1, 2, 3, 4 or 5 substituents independently selected from OH, CN, SC(O)Ph, oxo (—O), SH, SO₂NH₂, SO₂(C₁-C₄)alkyl, SO₂NH(C₁-C₄)alkyl, halogen, carbonyl, thiol, cyano, NH₂, NH(C₁-C₄)alkyl, N[(C₁-C₄)alkyl]₂, C(O)NH₂, COOH, COOMe, acetyl, (C₁-C₈)alkyl, O(C₁-C₈)alkyl (i.e., C₁-C₈alkoxy), O(C₁-C₅)haloalkyl, (C₂-C₅)alkenyl, (C₂-C₅)alkynyl, haloalkyl, thioalkyl, cyanomethylene, alkylaminyl, aryl, heteroaryl, substituted aryl, NH₂—C(O)-alkylene, NH(Me)-C(O)-alkylene, CH₂—C(O)-alkyl, C(O)-alkyl, alkylcarbonylaminyl, CH₂—[CH(OH)]ₘ—(CH₂)ₚ—OH, CH₂—[CH(OH)]ₘ—(CH₂)ₚ—NH₂ or CH₂-aryl-alkoxy, where “m” and “p” are independently 1, 2, 3, 4, 5 or 6.

Oxygen protecting groups are well known in the art and include those described in detail in Greene's Protecting Groups in Organic Synthesis, P. G. M. Wuts, 5^{th} Edition, John Wiley & Sons, 2014, incorporated herein by reference.

Exemplary oxygen protecting groups include, but are not limited to, methyl, t-butyloxycarbonyl (BOC or Boc), methoxylmethyl (MOM), methylthiomethyl (MTM), t-butylthiomethyl, (phenyldimethylsilyl) methoxymethyl (SMOM), benzyloxymethyl (BOM), p-methoxybenzyloxymethyl (PMBM), (4-methoxyphenoxy)methyl (p-AOM), guaiacolmethyl (GUM), t-butoxymethyl, 4-pentenyloxymethyl (POM), siloxymethyl, 2-methoxyethoxymethyl (MEM), 2,2,2-trichloroethoxymethyl, bis(2-chloroethoxy)methyl, 2-(trimethylsilyl) ethoxymethyl (SEMOR), tetrahydropyranyl (THP), 3-bromotetrahydropyranyl, tetrahydrothiopyranyl, 1-methoxycyclohexyl, 4-methoxytetrahydropyranyl (MTHP), 4-methoxytetrahydrothiopyranyl, 4-methoxytetrahydrothiopyranyl S,S-dioxide, 1-[(2-chloro-4-methyl)phenyl]-4-methoxypiperidin-4-yl(CTMP), 1,4-dioxan-2-yl, tetrahydrofuranyl, tetrahydrothiofuranyl, 2,3,3a,4,5,6,7,7a-octahydro-7,8,8-trimethyl-4,7-methanobenzofuran-2-yl, 1-ethoxyethyl, 1-(2-chloroethoxy)ethyl, 1-methyl-1-methoxyethyl, 1-methyl-1-benzyloxyethyl, 1-methyl-1-benzyloxy-2-fluoroethyl, 2,2,2-trichloroethyl, 2-trimethylsilylethyl, 2-(phenylselenyl)ethyl, t-butyl, allyl, p-chlorophenyl, p-methoxyphenyl, 2,4-dinitrophenyl, benzyl (Bn), p-methoxybenzyl, 3,4-dimethoxybenzyl, 0-nitrobenzyl, p-nitrobenzyl, p-halobenzyl, 2,6-dichlorobenzyl, p-cyanobenzyl, p-phenylbenzyl, 2-picolyl, 4-picolyl, 3-methyl-2-picolyl N-oxido, diphenylmethyl, p,p′-dinitrobenzhydryl, 5-dibenzosuberyl, triphenylmethyl, α-naphthyldiphenylmethyl, p-methoxyphenyldiphenylmethyl, di(p-methoxyphenyl)phenylmethyl, tri(p-methoxyphenyl)methyl, 4-(4′-bromophenacyloxyphenyl)diphenylmethyl, 4,4′,4″-tris(4,5-dichlorophthalimidophenyl)methyl, 4,4′,4″-tris(levulinoyloxyphenyl)methyl, 4,4′,4″-tris(benzoyloxyphenyl)methyl, 3-(imidazol-1-yl)bis(4′,4″-dimethoxyphenyl)methyl, 1,1-bis(4-methoxyphenyl)-1′-pyrenylmethyl, 9-anthryl, 9-(9-phenyl)xanthenyl, 9-(9-phenyl-10-oxo)anthryl, 1,3-benzodisulfuran-2-yl, benzisothiazolyl S,S-dioxido, trimethylsilyl (TMS), triethylsilyl (TES), triisopropylsilyl (TIPS), dimethylisopropylsilyl (IPDMS), diethylisopropylsilyl (DEIPS), dimethylthexylsilyl, t-butyldimethylsilyl (TBDMS), t-butyldiphenylsilyl (TBDPS), tribenzylsilyl, tri-p-xylylsilyl, triphenylsilyl,diphenylmethylsilyl (DPMS), t-butylmethoxyphenylsilyl (TBMPS), formate, acetate, chloroacetate, dichloroacetate, trichloroacetate, trifluoroacetate, methoxyacetate, triphenylmethoxyacetate, phenoxyacetate, p-chlorophenoxyacetate, 3-phenylpropionate, 4-oxopentanoate (levulinate), 4,4-(ethylenedithio) pentanoate (levulinoyldithioacetal), adamantoate, crotonate, 4-methoxycrotonate, benzoate, p-phenylbenzoate, 2,4,6-trimethylbenzoate (mesitoate), alkyl methyl carbonate, 9-fluorenylmethyl carbonate (Fmoc), alkyl ethyl carbonate, alkyl 2,2,2-trichloroethyl carbonate (Troc), 2-(trimethylsilyl)ethyl carbonate (TMSEC), 2-(phenylsulfonyl) ethyl carbonate (Psec), 2-(triphenylphosphonio) ethyl carbonate (Peoc), alkyl isobutyl carbonate, alkyl vinyl carbonate alkyl allyl carbonate, alkyl p-nitrophenyl carbonate, alkyl benzyl carbonate, alkyl p-methoxybenzyl carbonate, alkyl 3,4-dimethoxybenzyl carbonate, alkyl o-nitrobenzyl carbonate, alkyl p-nitrobenzyl carbonate, alkyl S-benzyl thiocarbonate, 4-ethoxy-1-napththyl carbonate, methyl dithiocarbonate, 2-iodobenzoate, 4-azidobutyrate, 4-nitro-4-methylpentanoate, o-(dibromomethyl)benzoate, 2-formylbenzenesulfonate, 2-(methylthiomethoxy)ethyl, 4-(methylthiomethoxy) butyrate, 2-(methylthiomethoxymethyl)benzoate, 2,6-dichloro-4-methylphenoxyacetate, 2,6-dichloro-4-(1,1,3,3-tetramethylbutyl) phenoxyacetate, 2,4-bis(1,1-dimethylpropyl) phenoxyacetate, chlorodiphenylacetate, isobutyrate, monosuSP3inoate, (E)-2-methyl-2-butenoate, 0-(methoxyacyl)benzoate, α-naphthoate, nitrate, alkylN,N,N′,N′-tetramethylphosphorodiamidate, alkyl N-phenylcarbamate, borate, dimethylphosphinothioyl, alkyl 2,4-dinitrophenylsulfenate, sulfate, methanesulfonate (mesylate), benzylsulfonate, and tosylate (Ts).

In some embodiments of any one of the aspects described herein, oxygen protecting group is benzyl, benzoyl, 2,6-dichlorobenzyl, t-butyldimethylsilyl, t-butyldiphenylsilyl, mesylate, tosylate, 4,4′-dimethoxytrityl (DMT), 9-phenylxanthine-9-yl (Pixyl) and 9-(p-methoxyphenyl) xanthine-9-yl (MOX). In certain embodiments, the hydroxyl protecting group is selected from acetyl, benzyl, t-butyldimethylsilyl, t-butyldiphenylsilyl and dimethoxytrityl wherein a more preferred hydroxyl protecting group is 4,4′-dimethoxytrityl.

The terms “protected hydroxyl” and “protected hydroxyl” as used herein mean a group of the formula —OR^{Pro}, wherein R^{Pro} is an oxygen protecting group as defined herein.

### Nitrogen Protecting Groups

Some embodiments of the various aspects described herein include a nitrogen protecting group (also referred to as an amino protecting group herein). Nitrogen protecting groups include, but are not limited to, —OH, —OR^{NP1}, —N(R^{NP2})₂, —C(═O)R^{NP1}, —C(═O)N(R^{NP2})₂, —CO₂R^{NP1}, —SO₂R^{NP1}, —C(═NR^{NP2})R^{NP1}, —C(═NR^{NP2})OR^{NP1}, —C(═NR^{NP2}) N(R^{NP2})₂, —SO₂N(R^{NP2})₂, —SO₂R^{NP2}, —SO₂OR^{NP2}, —SOR^{NP1}, —C(═S) N(R^{NP2})₂, —C(═O)SR^{NP2}, —C(═S) SR^{NP2}, C₁₋₁₀ alkyl (e.g., aralkyl, heteroaralkyl), C₂₋₁₀ alkenyl, C₂₋₁₀ alkynyl, C₃₋₁₀ carbocyclyl, 3-14 membered heterocyclyl, C₆₋₁₄ aryl, and 5-14 membered heteroaryl groups, where each R^{NP1} is independently C₁₋₁₀ alkyl, C₁₋₁₀ perhaloalkyl, C₂₋₁₀ alkenyl, C₂₋₁₀ alkynyl, heteroC₁₋₁₀ alkyl, heteroC₂₋₁₀alkenyl, heteroC₂₋₁₀alkynyl, C₃₋₁₀ carbocyclyl, 3-14 membered heterocyclyl, C₆₋₁₄ aryl, or 5-14 membered heteroaryl, or two R^{NP1} groups are joined to form a 3-14 membered heterocyclyl or 5-14 membered heteroaryl ring; and each R^{NP2} is independently hydrogen, C₁₋₁₀ alkyl, C₁₋₁₀ perhaloalkyl, C₂₋₁₀ alkenyl, C₂₋₁₀ alkynyl, heteroC₁₋₁₀ alkyl, heteroC₂₋₁₀ alkenyl, heteroC₂₋₁₀ alkynyl, C₃₋₁₀ carbocyclyl, 3-14 membered heterocyclyl, C₆₋₁₄ aryl, and 5-14 membered heteroaryl, or two R^{SP3} groups are joined to form a 3-14 membered heterocyclyl or 5-14 membered heteroaryl ring, and wherein each alkyl, alkenyl, alkynyl, carbocyclyl, heterocyclyl, aralkyl, aryl, and heteroaryl of R^{NP1} and R^{NP2} can be optionally substituted with 1, 2, 3, 4 or 5 substituents independently selected from OH, CN, SC(O)Ph, oxo (═O), SH, SO₂NH₂, SO₂(C₁-C₄)alkyl, SO₂NH(C₁-C₄)alkyl, halogen, carbonyl, thiol, cyano, NH₂, NH(C₁-C₄)alkyl, N[(C₁-C₄)alkyl]₂, C(O)NH₂, COOH, COOMe, acetyl, (C₁-C₈)alkyl, O(C₁-C₅)alkyl (i.e., C₁-C₈alkoxy), O(C₁-C₈)haloalkyl, (C₂-C₈)alkenyl, (C₂-C₈)alkynyl, haloalkyl, thioalkyl, cyanomethylene, alkylaminyl, aryl, heteroaryl, substituted aryl, NH₂—C(O)-alkylene, NH(Me)-C(O)-alkylene, CH₂C(O)-alkyl, C(O)-alkyl, alkylcarbonylaminyl, CH₂—[CH(OH)]ₘ—(CH₂)ₚ—OH, CH₂—[CH(OH)]ₘ—(CH₂)ₚ—NH₂ or CH₂-aryl-alkoxy, where “m” and “p” are independently 1, 2, 3, 4, 5 or 6.

Nitrogen protecting groups are well known in the art and include those described in detail in Greene's Protecting Groups in Organic Synthesis, P. G. M. Wuts, 5th Edition, John Wiley & Sons, 2014, incorporated herein by reference.

Exemplary amide (e.g., —C(═O)R^{NP1}) nitrogen protecting groups include, but are not limited to, formamide, acetamide, chloroacetamide, trichloroacetamide, trifluoroacetamide, N-phenylacetamide, 3-phenylpropanamide, picolinamide, 3-pyridylcarboxamide, benzoylphenylalanyl derivative, benzamide, p-phenylbenzamide, o-nitophenylacetamide, o-nitrophenoxyacetamide, acetoacetamide, (N′-dithiobenzyloxy acylamino) acetamide, 3-(p-hydroxylphenyl) propanamide, 3-(o-nitrophenyl) propanamide, 2-methyl-2-(o-nitrophenoxy) propanamide, 2-methyl-2-(o-phenylazophenoxy) propanamide, 4-chlorobutanamide, 3-methyl-3-nitrobutanamide, 0-nitrocinnamide, N-acetylmethionine derivative, o-nitrobenzamide, and o-(benzoyloxymethyl)benzamide.

Exemplary carbamate (e.g., —C(═O) OR^{NP1}) nitrogen protecting groups include, but are not limited to, methyl carbamate, ethyl carbamate, 9-fluorenylmethyl carbamate (Fmoc), 9-(2-sulfo) fluorenylmethyl carbamate, 9-(2,7-dibromo) fluoroenylmethyl carbamate, 2,7-di-t-butyl-[9-(10,10-dioxo-10,10,10,10-tetrahydrothioxanthyl)]methyl carbamate (DBD-Tmoc), 4-methoxyphenacyl carbamate (Phenoc), 2,2,2-trichloroethyl carbamate (Troc), 2-trimethylsilylethyl carbamate (Teoc), 2-phenylethyl carbamate (hZ), 1-(1-adamantyl)-1-methylethyl carbamate (Adpoc), 1,1-dimethyl-2-haloethyl carbamate, 1,1-dimethyl-2,2-dibromoethyl carbamate (DB-t-BOC), 1,1-dimethyl-2,2,2-trichloroethyl carbamate (TCBOC), 1-methyl-1-(4-biphenylyl)ethyl carbamate (Bpoc), 1-(3,5-di-t-butylphenyl)-1-methylethyl carbamate (t-Bumeoc), 2-(2′- and 4′-pyridyl)ethyl carbamate (Pyoc), 2-(N,N-dicyclohexylcarboxamido)ethyl carbamate, t-butyl carbamate (BOC or Boc), 1-adamantyl carbamate (Adoc), vinyl carbamate (Voc), allyl carbamate (Alloc), 1-isopropylallyl carbamate (Ipaoc), cinnamyl carbamate (Coc), 4-nitrocinnamyl carbamate (Noc), 8-quinolyl carbamate, N-hydroxylpiperidinyl carbamate, alkyldithio carbamate, benzyl carbamate (Cbz), p-methoxybenzyl carbamate (Moz), p-nitobenzyl carbamate, p-bromobenzyl carbamate, p-chlorobenzyl carbamate, 2,4-dichlorobenzyl carbamate, 4-methylsulfinylbenzyl carbamate (Msz), 9-anthrylmethyl carbamate, diphenylmethyl carbamate, 2-methylthioethyl carbamate, 2-methylsulfonylethyl carbamate, 2-(p-toluenesulfonyl)ethyl carbamate, [2-(1,3-dithianyl)]methyl carbamate (Dmoc), 4-methylthiophenyl carbamate (Mtpc), 2,4-dimethylthiophenyl carbamate (Bmpc), 2-phosphonioethyl carbamate (Peoc), 2-triphenylphosphonioisopropyl carbamate (Ppoc), 1,1-dimethyl-2-cyanoethyl carbamate, m-chloro-p-acyloxybenzyl carbamate, p-(dihydroxylboryl)benzyl carbamate, 5-benzisoxazolylmethyl carbamate, 2-(trifluoromethyl)-6-chromonylmethyl carbamate (Tcroc), m-nitrophenyl carbamate, 3,5-dimethoxybenzyl carbamate, o-nitrobenzyl carbamate, 3,4-dimethoxy-6-nitrobenzyl carbamate, phenyl(o-nitrophenyl)methyl carbamate, t-amyl carbamate, S-benzyl thiocarbamate, p-cyanobenzyl carbamate, cyclobutyl carbamate, cyclohexyl carbamate, cyclopentyl carbamate, cyclopropylmethyl carbamate, p-decyloxybenzyl carbamate, 2,2-dimethoxyacylvinyl carbamate, o-(N,N-dimethylcarboxamido)benzyl carbamate, 1,1-dimethyl-3-(N,N-dimethylcarboxamido) propyl carbamate, 1,1-dimethylpropynyl carbamate, di(2-pyridyl)methyl carbamate, 2-furanylmethyl carbamate, 2-iodoethyl carbamate, isoborynl carbamate, isobutyl carbamate, isonicotinyl carbamate, p-(p′-methoxyphenylazo)benzyl carbamate, 1-methylcyclobutyl carbamate, 1-methylcyclohexyl carbamate, 1-methyl-1-cyclopropylmethyl carbamate, 1-methyl-1-(3,5-dimethoxyphenyl)ethyl carbamate, 1-methyl-1-(p-phenylazophenyl)ethyl carbamate, 1-methyl-1-phenylethyl carbamate, 1-methyl-1-(4-pyridyl)ethyl carbamate, phenyl carbamate, p-(phenylazo)benzyl carbamate, 2,4,6-tri-t-butylphenyl carbamate, 4-(trimethylammonium)benzyl carbamate, and 2,4,6-trimethylbenzyl carbamate.

Exemplary sulfonamide (e.g., —S(═O)₂R^{NP1}) nitrogen protecting groups include, but are not limited to, such as p-toluenesulfonamide (Ts), benzenesulfonamide, 2,3,6,-trimethyl-4-methoxybenzenesulfonamide (Mtr), 2,4,6-trimethoxybenzenesulfonamide (Mtb), 2,6-dimethyl-4-methoxybenzenesulfonamide (Pme), 2,3,5,6-tetramethyl-4-methoxybenzenesulfonamide (Mte), 4-methoxybenzenesulfonamide (Mbs), 2,4,6-trimethylbenzenesulfonamide (Mts), 2,6-dimethoxy-4-methylbenzenesulfonamide (iMds), 2,2,5,7,8-pentamethylchroman-6-sulfonamide (Pmc), methanesulfonamide (Ms), β-trimethylsilylethanesulfonamide (SES), 9-anthracenesulfonamide, 4-(4′,8′-dimethoxynaphthylmethyl)benzenesulfonamide (DNMBS), benzylsulfonamide, trifluoromethylsulfonamide, and phenacylsulfonamide.

Additional exemplary nitrogen protecting groups include, but are not limited to, phenothiazinyl-(10)-acyl derivative, N′-p-toluenesulfonylaminoacyl derivative, N′-phenylaminothioacyl derivative, N-benzoylphenylalanyl derivative, N-acetylmethionine derivative, 4,5-diphenyl-3-oxazolin-2-one, N-phthalimide, N-dithiasuNP2inimide (Dts), N-2,3-diphenylmaleimide, N-2,5-dimethylpyrrole, N-1,1,4,4-tetramethyldisilylazacyclopentane adduct (STABASE), 5-substituted 1,3-dimethyl-1,3,5-triazacyclohexan-2-one, 5-substituted 1,3-dibenzyl-1,3,5-triazacyclohexan-2-one, 1-substituted 3,5-dinitro-4-pyridone, N-methylamine, N-allylamine, N-[2-(trimethylsilyl) ethoxy]methylamine (SEM), N-3-acetoxypropylamine, N-(1-isopropyl-4-nitro-2-oxo-3-pyroolin-3-yl)amine, quaternary ammonium salts, N-benzylamine, N-di (4-methoxyphenyl)methylamine, N-5-dibenzosuberylamine, N-triphenylmethylamine (Tr), N-[(4-methoxyphenyl)diphenylmethyl]amine (MMTr), N-9-phenylfluorenylamine (PhF), N-2,7-dichloro-9-fluorenylmethyleneamine, N-ferrocenylmethylamino (Fcm), N-2-picolylamino N′-oxide, N-1,1-dimethylthiomethyleneamine, N-benzylideneamine, N-p-methoxybenzylideneamine, N-diphenylmethyleneamine, N-[(2-pyridyl) mesityl]methyleneamine, N—(N′,N′-dimethylaminomethylene)amine, N,N′-isopropylidenediamine, N-p-nitrobenzylideneamine, N-salicylideneamine, N-5-chlorosalicylideneamine, N-(5-chloro-2-hydroxylphenyl)phenylmethyleneamine, N-cyclohexylideneamine, N-(5,5-dimethyl-3-oxo-1-cyclohexenyl)amine, N-borane and N-diphenylborinic acid derivative, N-[phenyl(pentNP1cylchromium- or tungsten) acyl]amine, N-copper chelate, N-zinc chelate, N-nitroamine, N-nitrosoamine, amine N-oxide, diphenylphosphinamide (Dpp), dimethylthiophosphinamide (Mpt), diphenylthiophosphinamide (Ppt), dialkyl phosphoramidates, dibenzyl phosphoramidate, diphenyl phosphoramidate, benzenesulfenamide, 0-nitrobenzenesulfenamide (Nps), 2,4-dinitrobenzenesulfenamide, pentachlorobenzenesulfenamide, 2-nitro-4-methoxybenzenesulfenamide, triphenylmethylsulfenamide, and 3-nitropyridinesulfenamide (Npys).

### Sulfur Protecting Groups

Some embodiments of the various aspects described herein include sulfur protecting group (also referred to as a thiol protecting group herein). Sulfur protecting groups include, but are not limited to, —R^{SP1}, —N(R^{SP2})₂, —C(═O)SR^{SP1}, —C(═O)R^{SP1}, —CO₂R^{SP1}, C(═O)N(R^{SP2})₂, —C(═NR^{SP2})R^{SP1}, —C(═NR^{SP2})OR^{SP1}, —C(═NR^{SP2}) N(R^{SP2})₂, —S(═O)R^{SP1}, —SO₂R^{SP1}, —Si(R^{SP1})₃, —P(R^{SP3})₂, —P(R^{SP3})⁺₃ X⁻, —P(OR^{SP3})₂, —P(OR^{SP3})⁺₃ X⁻, —P(═O)(R^{SP1})₂, —P(═O)(OR^{SP3})₂, and —P(═O)(N(R^{SP2})₂)₂, wherein

X⁻ is a counterion; each R^{SP1} is independently C₁₋₁₀ alkyl, C₁₋₁₀ perhaloalkyl, C₂₋₁₀ alkenyl, C₂₋₁₀ alkynyl, heteroC₁₋₁₀ alkyl, heteroC₂₋₁₀alkenyl, heteroC₂₋₁₀alkynyl, C₃₋₁₀ carbocyclyl, 3-14 membered heterocyclyl, C₆₋₁₄ aryl, or 5-14 membered heteroaryl, or two R^{SP1} groups are joined to form a 3-14 membered heterocyclyl or 5-14 membered heteroaryl ring; each R^{SP2} is hydrogen, —OH, OR^{SP1}, —N(R^{SP3})₂, CN, —C(═O)R^{SP1}, C(═O)N(R^{SP3})₂, —CO₂R^{SP1} SO₂R^{SP1}, —C(═NR^{SP3})OR^{SP1}, —C(═NR^{SP3}) N(R^{SP3})₂, —SO₂N(R^{SP3})₂, —SO₂R^{SP3}, —SO₂OR^{SP3}, —SOR^{SP1}, —C(═S) N(R^{SP3})₂, —C(═O)SR^{SP3}, —C(═S) SR^{SP3}, —P(═O) (R^{SP1})₂, —P(═O)(OR^{SP3})₂, —P(═O)(N(R^{SP3})₂)₂, C₁₋₁₀ alkyl, C₁₋₁₀ perhaloalkyl, C₂₋₁₀ alkenyl, C₂₋₁₀ alkynyl, heteroC₁₋₁₀alkyl, heteroC₂₋₁₀alkenyl, heteroC₂₋₁₀alkynyl, C₃₋₁₀ carbocyclyl, 3-14 membered heterocyclyl, C₆₋₁₄ aryl, and 5-14 membered heteroaryl, or two R^{SP2} groups are joined to form a 3-14 membered heterocyclyl or 5-14 membered heteroaryl ring; and each R^{SP3} is independently hydrogen, C₁₋₁₀ alkyl, C₁₋₁₀ perhaloalkyl, C₂₋₁₀ alkenyl, C₂₋₁₀ alkynyl, heteroC₁₋₁₀ alkyl, heteroC₂₋₁₀ alkenyl, heteroC₂₋₁₀ alkynyl, C₃₋₁₀ carbocyclyl, 3-14 membered heterocyclyl, C₆₋₁₄ aryl, and 5-14 membered heteroaryl, or two R^{SP3} groups are joined to form a 3-14 membered heterocyclyl or 5-14 membered heteroaryl ring; and wherein each alkyl, alkenyl, alkynyl, carbocyclyl, heterocyclyl, aralkyl, aryl, and heteroaryl of R^{SP1}, R^{SP2} and R^{SP3} can be optionally substituted with 1, 2, 3, 4 or 5 substituents independently selected from OH, CN, SC(O)Ph, oxo (═O), SH, SO₂NH₂, SO₂(C₁-C₄)alkyl, SO₂NH(C₁-C₄)alkyl, halogen, carbonyl, thiol, cyano, NH₂, NH(C₁-C₄)alkyl, N[(C₁-C₄)alkyl]₂, C(O)NH₂, COOH, COOMe, acetyl, (C₁-C₈)alkyl, O(C₁-C₈)alkyl (i.e., C₁-C₈alkoxy), O(C₁-C₈)haloalkyl, (C₂-C₈)alkenyl, (C₂-C₈)alkynyl, haloalkyl, thioalkyl, cyanomethylene, alkylaminyl, aryl, heteroaryl, substituted aryl, NH₂—C(O)-alkylene, NH(Me)-C(O)-alkylene, CH₂C(O)— alkyl, C(O)-alkyl, alkylcarbonylaminyl, CH₂—[CH(OH)]ₘ—(CH₂)ₚ—OH, CH₂—[CH(OH)]ₘ—(CH₂)ₚ—NH₂ or CH₂-aryl-alkoxy, where “m” and “p” are independently 1, 2, 3, 4, 5 or 6.

Sulfur protecting groups are well known in the art and include those described in detail in Greene's Protecting Groups in Organic Synthesis, P. G. M. Wuts, 5th Edition, John Wiley & Sons, 2014, incorporated herein by reference.

Exemplary embodiments of the various aspects described herein can be illustrated by the following numbered embodiments:

Embodiment 1: A double-stranded RNA (dsRNA) comprising an antisense strand and a sense strand complementary to the antisense strand, wherein the antisense strand comprises at its 3′-end a first ligand, wherein the antisense strand comprises at least one nuclease resistant modification at its 3′-end and at least one nuclease resistant modification at its 5′-end, and wherein the dsRNA has a double-stranded region of at least about 15 base-pairs.

Embodiment 2: The dsRNA of Embodiment 1, wherein the sense strand comprises at least one nuclease resistant modification at its 5′-end.

Embodiment 3: The dsRNA of Embodiment 1 or 2, wherein the sense strand comprises at least one nuclease resistant modification at its 3′-end and at least one nuclease resistant modification at its 5′-end.

Embodiment 4: The dsRNA of any one of the preceding Embodiments, wherein the at least one nuclease resistant modification is a modified internucleoside linkage, a modified sugar moiety or a modified nucleobase.

Embodiment 5: The dsRNA of any one of the preceding Embodiments, wherein the at least one nuclease resistant modification is a phosphorothioate internucleoside linkage, a phosphorodithioate internucleoside linkage, a 2′-5′-linked nucleotide, or a L-nucleotide.

Embodiment 6: The dsRNA of any one of the preceding Embodiments, wherein the dsRNA comprises at least 4 phosphorothioate internucleoside linkages, e.g., at least 6 phosphorothioate internucleoside linkages, at least 8 phosphorothioate internucleoside linkages or at least 10 phosphorothioate internucleoside linkages.

Embodiment 7: The dsRNA of any one of the preceding claims, wherein the antisense strand comprises at least two, e.g., three, four, five, six or more phosphorothioate internucleoside linkages.

Embodiment 8: The dsRNA of any one of the preceding Embodiments, wherein the antisense strand comprises a phosphorothioate internucleoside linkage between positions 1 and 2, counting from the 3′-end of the strand, and a phosphorothioate internucleoside linkage between positions 1 and 2, counting from the 5′-end of the strand.

Embodiment 9: The dsRNA of any one of the preceding Embodiments, wherein the antisense strand comprises a phosphorothioate internucleoside linkage between positions 1 and 2, and between positions 2 and 3, counting from the 3′-end of the strand, and a phosphorothioate internucleoside linkage between positions 1 and 2, counting from the 5′-end of the strand.

Embodiment 10: The dsRNA of any one of the preceding Embodiments, wherein the antisense strand comprises a phosphorothioate internucleoside linkage between positions 1 and 2, and between positions 2 and 3, counting from the 3′-end of the strand, and a phosphorothioate internucleoside linkage between positions 1 and 2, and between positions 2 and 3, counting from the 5′-end of the strand.

Embodiment 11: The dsRNA of any one of the preceding Embodiments, wherein the antisense strand comprises a phosphorothioate internucleoside linkage between positions 1 and 2, between positions 2 and 3, and between positions 3 and 4, counting from the 3′-end of the strand, and a phosphorothioate internucleoside linkage between positions 1 and 2, counting from the 5′-end of the strand.

Embodiment 12: The dsRNA of any one of the preceding Embodiments, wherein the antisense strand comprises a phosphorothioate internucleoside linkage between positions 1 and 2, counting from the 3′-end of the strand, and a phosphorothioate internucleoside linkage between positions 1 and 2, and between positions 2 and 3, counting from the 5′-end of the strand.

Embodiment 13: The dsRNA of any one of the preceding Embodiments, wherein the antisense strand comprises a phosphorothioate internucleoside linkage between positions 1 and 2, counting from the 3′-end of the strand, and a phosphorothioate internucleoside linkage between positions 1 and 2, between positions 2 and 3, and between positions 3 and 4, counting from the 5′-end of the strand.

Embodiment 14: The dsRNA of any one of the preceding Embodiments, wherein the sense strand comprises at least one, e.g., two, three, four or more phosphorothioate internucleoside linkages.

Embodiment 15: The dsRNA of any one of the preceding Embodiments, wherein the sense strand comprises a phosphorothioate internucleoside linkage between positions 1 and 2, counting from 5′-end of the strand.

Embodiment 16: The dsRNA of any one of the preceding Embodiments, wherein the sense strand comprises a phosphorothioate internucleoside linkage between positions 1 and 2, counting from 5′-end of the strand, and between positions 1 and 2, counting from 3′-end of the strand.

Embodiment 17: The dsRNA any one of the preceding Embodiments, wherein the sense strand comprises a phosphorothioate internucleoside linkage between positions 1 and 2, and between positions 2 and 3, counting from 5′-end of the strand.

Embodiment 18: The dsRNA any one of the preceding Embodiments, wherein the sense strand comprises a phosphorothioate internucleoside linkage between positions 1 and 2, and between positions 2 and 3, counting from 5′-end of the strand, and between positions 1 and 2, and between positions 2 and 3, counting from 3′-end of the strand.

Embodiment 19: The dsRNA of any one of the preceding Embodiments, wherein the ligand is linked to 3′-hydroxyl of the nucleotide at position 1, counting from 3′-end, of antisense strand.

Embodiment 20: The dsRNA of any one of the preceding Embodiments, wherein the first ligand is linked to the 3′-end of the antisense strand via a linker.

Embodiment 21: The dsRNA of Embodiment 20, wherein the linker is a hydrophobic linker.

Embodiment 22: The dsRNA of Embodiment 20 or 21, where the linker is linked to the 3′-end of the antisense strand via a phosphodiester or phosphorothioate internucleoside linkage.

Embodiment 23: The dsRNA of any one of Embodiments 20-22, wherein the linker is from about 5 Angstroms to about 250 Angstroms in length, e.g., from about 10 Angstroms to about 200 Angstroms, from about 15 Angstroms to about 150 Angstroms, from about 20 Angstroms to about 100 Angstroms, from about 25 Angstroms to about 75 Angstroms, from about 5 Angstroms to about 50 Angstroms, from about 10 Angstroms to about 40 Angstroms or from about 20 Angstroms to about 30 Angstroms in length.

Embodiment 24: The dsRNA of any one of Embodiments 20-23, wherein the linker has a chain length of at least 6 atoms (e.g., 6, 7, 8, 9, 10, 11, 12, 13, 14, 15, 16, 17, 18, 19, 20, 21, 22, 23, 24, 25, 26, 27, 28, 29 or 30 atoms).

Embodiment 25: The dsRNA of any one of Embodiments 20-24, wherein the linker comprises a hydrophobic carrier connected to a carrier.

Embodiment 26: The dsRNA of Embodiment 25, wherein the carrier comprises a hydrogen-bonding acceptor (e.g., a tertiary amide or tertiary amine).

Embodiment 27: The dsRNA of Embodiment 26, wherein the carrier comprises a pyrrolidine ring.

Embodiment 28: The dsRNA of any one of the preceding Embodiments, wherein the antisense strand is at least about 17, e.g., about 18, about 19, about 20, about 21, about 22, about 23, about 24, about 25, about 26, about 27, about 28, about 29, about 30 or more (e.g., about 17-42), nucleotides in length.

Embodiment 29: The dsRNA of any one of the preceding Embodiments, wherein the antisense strand is about 19, about 20, about 21, about 22, about 23, about 24, about 25 or about 26 nucleotides in length.

Embodiment 30: The dsRNA of any one of the preceding Embodiments, wherein the antisense strand is about 22, about 23 or about 25 nucleotides in length.

Embodiment 31: The dsRNA of any one of the preceding Embodiments, wherein the sense strand is at least about 15, about 16, e.g., about 17, about 18, about 19, about 20, about 21, about 22, about 23, about 24, about 25, about 26, or more (e.g., about 15-40), nucleotides in length.

Embodiment 32: The dsRNA of any one of the preceding Embodiments, wherein the sense strand is about 19, about 20, about 21, about 22, about 23, about 24 or about 25 nucleotides in length.

Embodiment 33: The dsRNA of any one of the preceding Embodiments, wherein the sense strand is about 21 nucleotides in length.

Embodiment 34: The dsRNA of any one of the preceding Embodiments, wherein:
(a) the sense strand is 15 nucleotides in length and the antisense strand is 18, 19, 20, 21, or 22 (e.g., 20) nucleotides in length;
(b) the sense strand is 19 nucleotides in length and the antisense strand is 19, 20, or 21 nucleotides in length;
(c) the sense strand is 20 nucleotides in length and the antisense strand is 20, 21, or 22 nucleotides in length;
(d) the sense strand is 21 nucleotides in length and the antisense strand is 21, 22, or 23 nucleotides in length; or
(e) the sense strand is 20-24 (e.g., 22) nucleotides in length and the antisense strand is 34-38 (e.g. 36) nucleotides in length.

Embodiment 35: The dsRNA of any one of the preceding Embodiments, wherein the dsRNA has a double-stranded region of at least about 15, e.g., about 16, about 17, about 18, about 19, about 20, about 21, about 22, about 23, about 24, about 25 or more base-pairs.

Embodiment 36: The dsRNA of any one of the preceding Embodiments, wherein the dsRNA has a double-stranded region of about 21 base-pairs.

Embodiment 37: The dsRNA of any one of the preceding Embodiments, wherein the sense strand is about 21 nucleotides in length and the antisense strand is about 21, about 22, about 23, about 24 or about 25 nucleotides in length, and wherein the dsRNA comprises a double-stranded region of at least 18, e.g., 19, 20 or 21 base-pairs.

Embodiment 38: The dsRNA of any one of the preceding Embodiments, wherein the dsRNA comprises at least one single-stranded overhang comprising 1-5 nucleotides (e.g., 1 or 2 nucleotides).

Embodiment 39: The dsRNA of any one of the preceding Embodiments, wherein the antisense strand comprises a single-stranded overhang at its 3′-end.

Embodiment 40: The dsRNA of any one of the preceding Embodiments, wherein the dsRNA comprises at least one blunt-end.

Embodiment 41: The dsRNA of any one of the preceding Embodiments, wherein the antisense strand comprises a blunt end at its 5′-end.

Embodiment 42: The dsRNA of any one of the preceding Embodiments, wherein the antisense strand comprises a single-stranded overhang at its 3′-end and a blunt end at its 5′-end.

Embodiment 43: The dsRNA of any one of Embodiments 38-42, wherein the antisense strand comprises at least one nuclease resistant modification in the single-stranded overhang.

Embodiment 44: The dsRNA of any one of Embodiments 38-43, wherein the antisense strand comprises at least one phosphorothioate internucleoside linkage in the single-stranded overhang.

Embodiment 45: The dsRNA of any one of the preceding Embodiments, wherein the first ligand is selected from the group consisting of peptides, centyrins, antibodies (e.g., antiCD-4 antibodies and antiCD-117 antibodies), antibody fragments, T-cell targeting ligands, B-cell targeting ligands, cancer cell targeting ligands (e.g., DUPA, folate, and RGD), spleen targeting functionalities, lung targeting functionalities, bone marrow targeting functionalities, phage display peptides, cell permeation peptides (CPPs), integrin ligands, multianionic ligands, multicationic ligands, monovalent and multivalent carbohydrates (e.g., GalNAc, mannose, mannose-6 phosphate, fucose, mucose, and mucose), kidney targeting ligands, BBB penetration ligands, lipids, and amino acids (e.g., L-amino acids, D-amino acids, and β-amino acids).

Embodiment 46: The dsRNA of any one of the preceding Embodiments, wherein the first ligand is a targeting ligand, a pharmacokinetics modulator (PK modulator) or an endosomolytic ligand.

Embodiment 47: The dsRNA of any one of the preceding Embodiments wherein the first ligand is a targeting ligand.

Embodiment 48: The dsRNA of any one of the preceding Embodiments, wherein the ligand comprises GalNAc.

Embodiment 49: The dsRNA of any one of the preceding Embodiments, wherein the ligand is

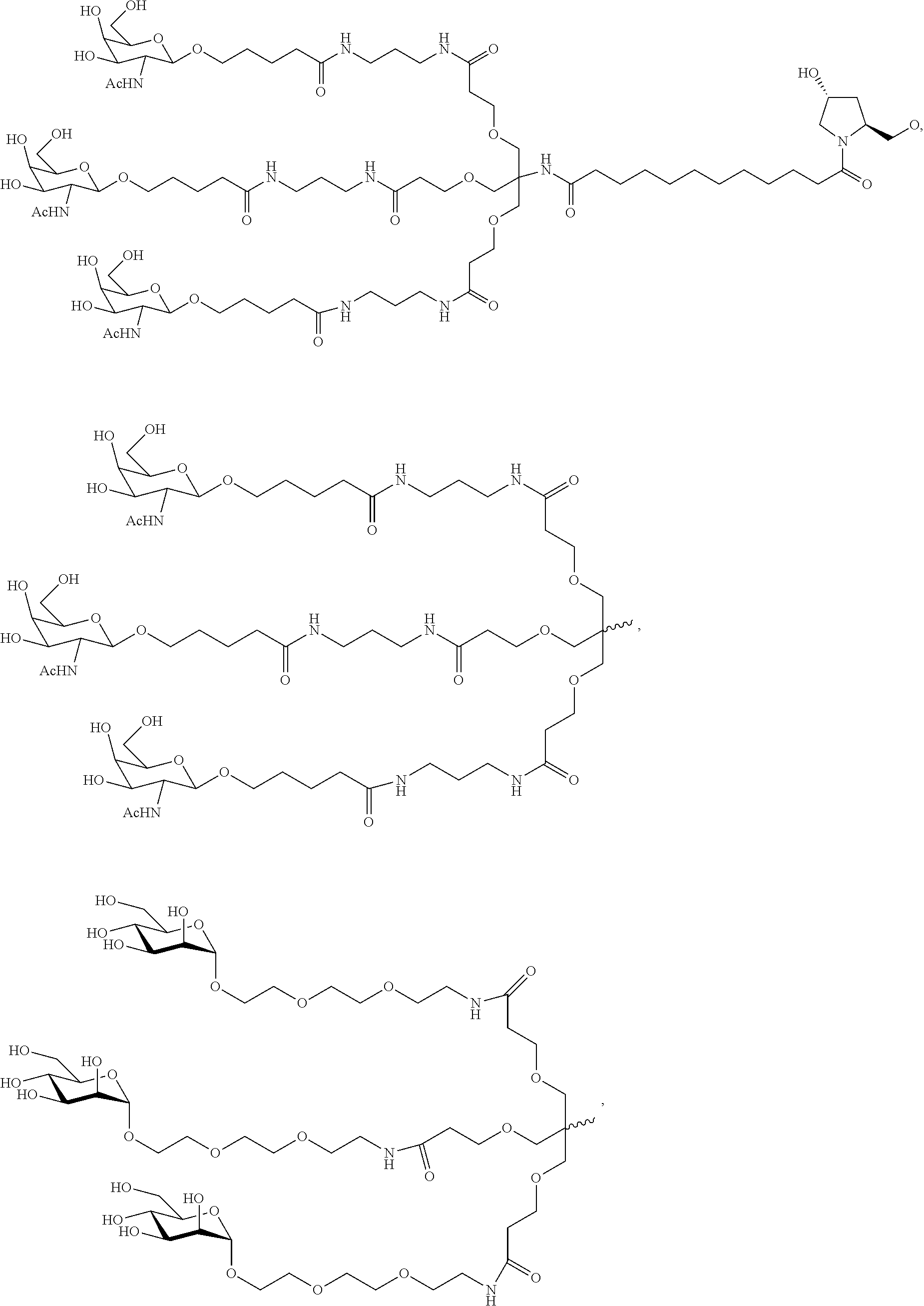

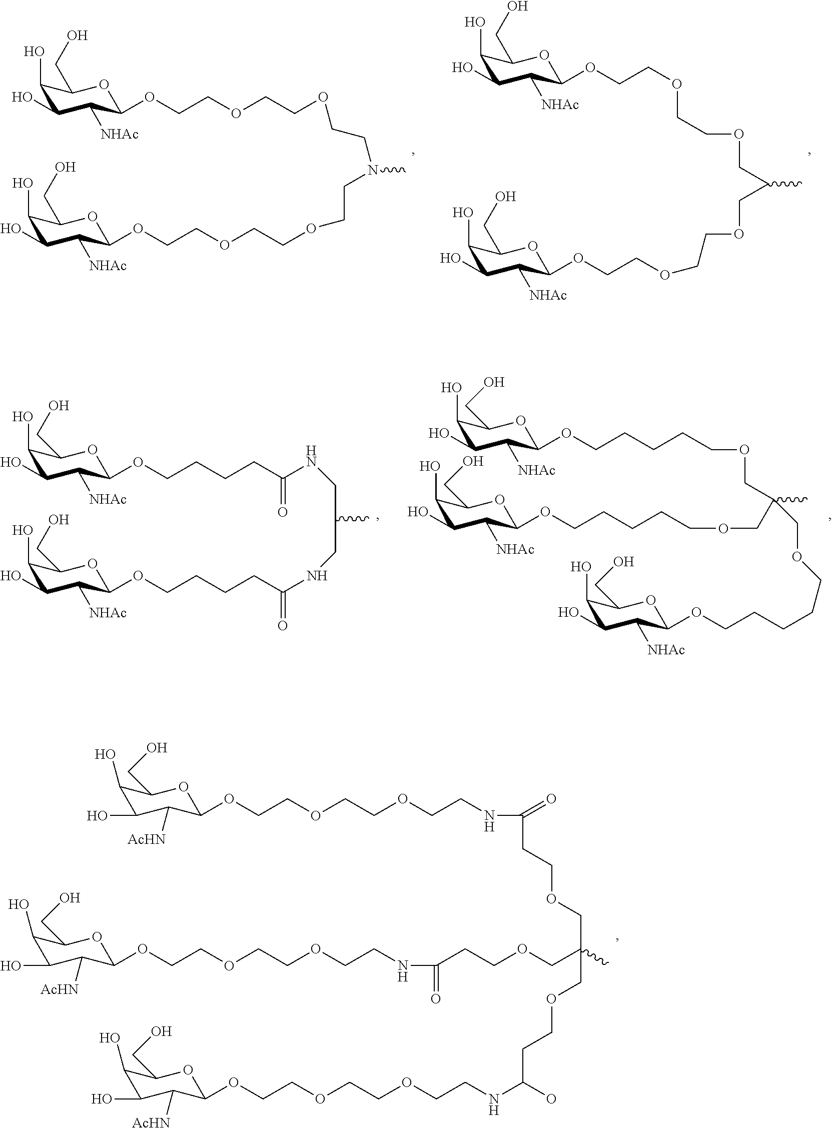

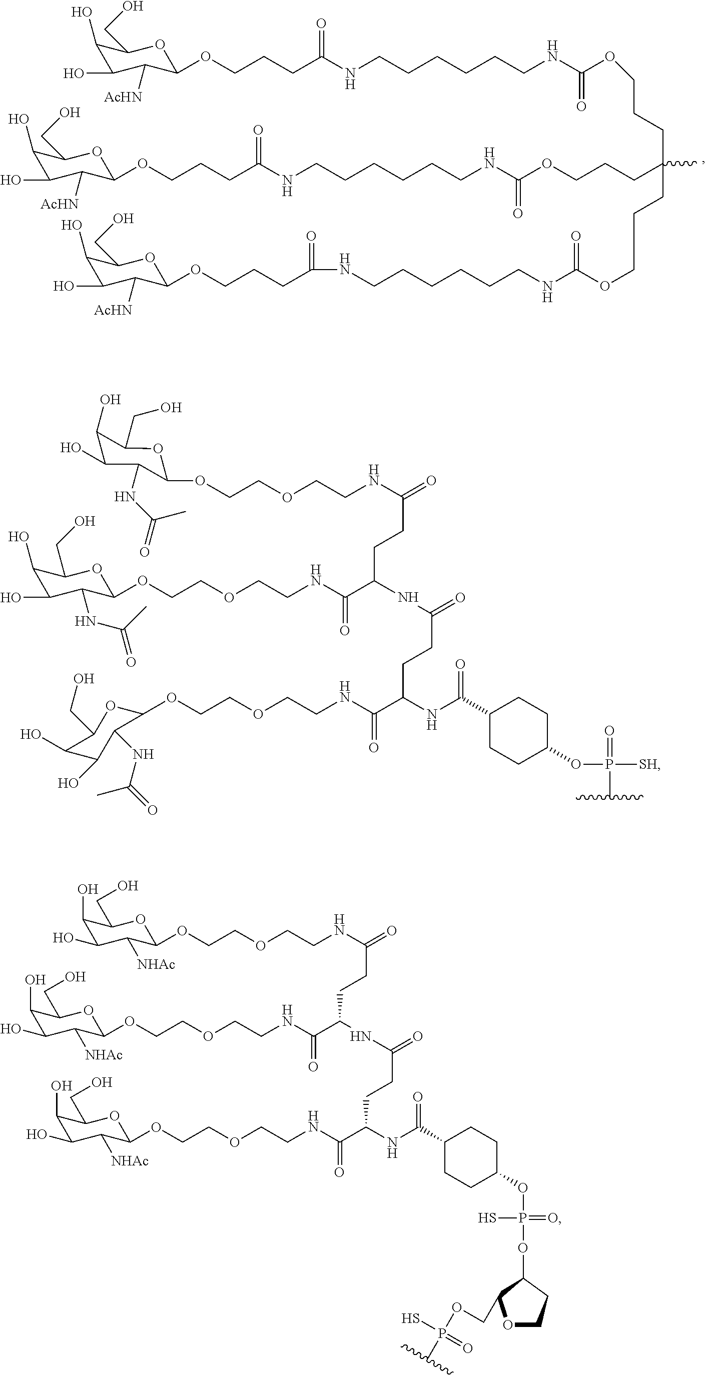

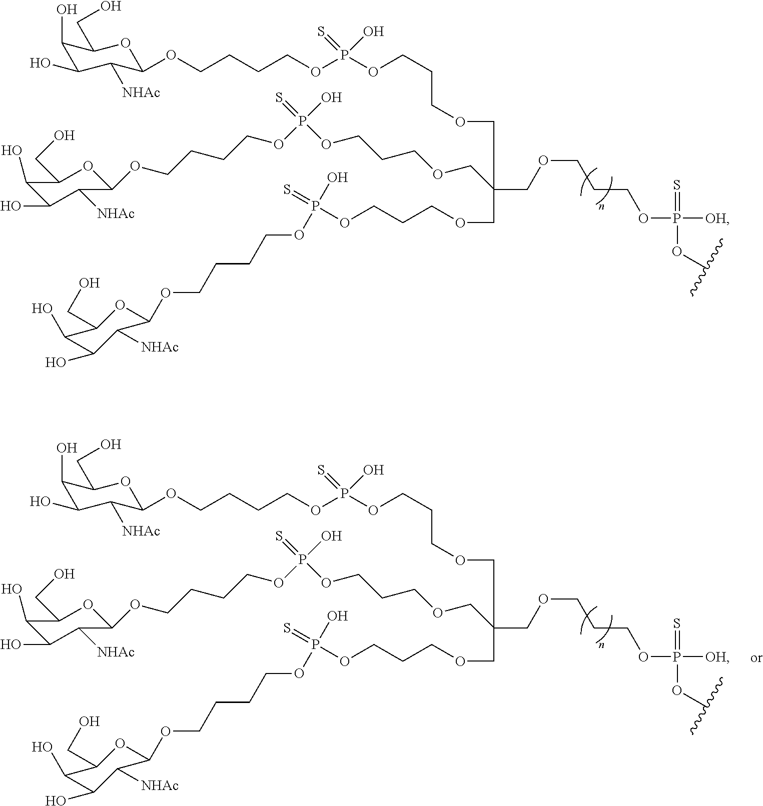

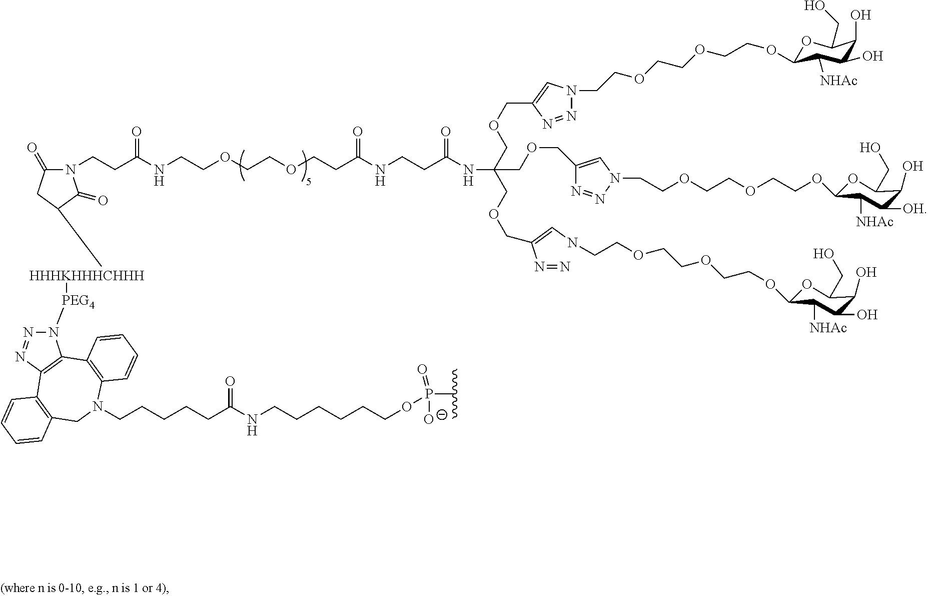

Embodiment 50: The daRNA of any one of the preceding Embodiments, wherein the daRNA comprises a second ligand.

Embodiment 51: The deRNA of Embodiment 50, wherein the second ligand is linked to the sense strand.

Embodiment 52: The deRNA of Embodiment 50 or 51, wherein the second ligand is linked to 3′-end of the sense strand.

Embodiment 53: The dsRNA of Embodiment 50 or 51, wherein the second ligand is linked to 5′-end of the sense strand.

Embodiment 54: The dsRNA of any one of Embodiments 50-53, wherein the second ligand is selected from the group consisting of peptides, centyrins, antibodies (e.g., antiCD-4 antibodies and antiCD-117 antibodies), antibody fragments, T-cell targeting ligands, B-cell targeting ligands, cancer cell targeting ligands (e.g., DUPA, folate, and RGD), spleen targeting functionalities, lung targeting functionalities, bone marrow targeting functionalities, phage display peptides, cell permeation peptides (CPPs), integrin ligands, multianionic ligands, multicationic ligands, monovalent and multivalent carbohydrates (e.g., GalNAc, mannose, mannose-6 phosphate, mucose, and mlucose), kidney targeting ligands, BBB penetration ligands, lipids, and amino acids (e.g., L-amino acids, D-amino acids, and β-amino acids).

Embodiment 55: The dsRNA of any one of Embodiments 50-54, wherein the second ligand is a PK modulator, a targeting ligand or an endosomolytic ligand.

Embodiment 56: The dsRNA of any one of Embodiments 50-55, wherein the second ligand is a PK modulator.

Embodiment 57: The dsRNA of any one of Embodiments 50-56, wherein the second ligand binds a serum protein, e.g., serum albumin.

Embodiment 58: The dsRNA of any one of Embodiments 50-57, wherein the second ligand comprises iodipamide, azapropazone, indomethacin, tiblone (TIB), 3-carboxy-4-methyl-5-propyl-2-furanpropanoic acid (CMPF), DIS, oxyphenbutazone, phenylbutazone, warfarin, indoxyl sulfate, diflunisal, halothane, ibuprofen, diazepam, propofol, or any combination thereof.

Embodiment 59: The dsRNA of any one of Embodiments 50-58, wherein the second ligand comprises ibuprofen.

Embodiment 60: The dsRNA of any one of Embodiments 50-59, wherein the first and second ligands are different.

Embodiment 61: The dsRNA of any one of Embodiments 50-60, wherein the first ligand is a targeting ligand and the second ligand is a PK modulator.

Embodiment 62: The dsRNA of any one of Embodiments 50-61, wherein the first ligand comprises GalNac and the second ligand comprises ibuprofen.

Embodiment 63: The dsRNA of any one of the preceding Embodiments, wherein the dsRNA comprises at least one, e.g., 2, 3, 4, 5, 6, 7, 8, 9, 10 or more 2′-fluoro nucleotide.

Embodiment 64: The dsRNA of any one of the preceding Embodiments, wherein the antisense strand comprises at least one, e.g., 2, 3, 4, 5 or more 2′-fluoro nucleotides.

Embodiment 65: The dsRNA of any one of the preceding Embodiments, wherein the antisense strand comprises a 2′-fluoro nucleotide at positions 2, 14 and 16, counting from the 5′-end of the antisense strand.

Embodiment 66: The dsRNA of any one of the preceding Embodiments, wherein the antisense strand comprises a 2′-fluoro nucleotide at positions 2, 6, 14 and 16, counting from the 5′-end of the antisense strand.

Embodiment 67: The dsRNA of any one of the preceding Embodiments, wherein the antisense strand comprises a 2′-fluoro nucleotide at positions 2, 6, 9, 14 and 16, counting from the 5′-end of the antisense strand.

Embodiment 68: The dsRNA of any one of the preceding Embodiments, wherein the antisense strand comprises a 2′-fluoro nucleotide at positions 2, 6, 8, 9, 14 and 16, counting from the 5′-end of the antisense strand.

Embodiment 69: The dsRNA of any one of the preceding Embodiments, wherein the antisense strand comprises at least one, e.g., 2, 3, 4, 5 or more 2′-fluoro nucleotides.

Embodiment 70: The dsRNA of any one of the preceding Embodiments, wherein the sense strand comprises a 2′-fluoro nucleotide at positions 7, 9 and 11, counting from the 5′-end of the sense strand or at positions 11, 13 and 15, counting from the 3′-end of the sense strand.

Embodiment 71: The dsRNA of any one of the preceding Embodiments, wherein the sense strand comprises a 2′-fluoro nucleotide at positions 7, 9, 10 and 11, counting from the 5′-end of the sense strand or at positions 11, 12, 13 and 15, counting from the 3′-end of the sense strand.

Embodiment 72: The dsRNA of any one of the preceding Embodiments, wherein the sense strand comprises a 2′-fluoro nucleotide at positions 9, 10, and 11, counting from the 5′-end of the sense strand or at positions 11, 12, and 13 counting from the 3′-end of the sense strand. Embodiment 73: The dsRNA of any one of the preceding Embodiments, the antisense strand comprises at least one, e.g., 2, 3, 4, 5, 6, 7 or more DNA nucleotides.

Embodiment 74: The dsRNA of any one of the preceding Embodiments, wherein the antisense strand comprises a DNA nucleotide at positions 2, 5, 7, and 12, counting from the 5′-end of the antisense strand.

Embodiment 75: The dsRNA of any one of the preceding Embodiments, wherein the antisense strand comprises a DNA nucleotide at positions 2, 5, 7, 12, and 14 counting from the 5′-end of the antisense strand.

Embodiment 76: The dsRNA of any one of the preceding Embodiments, wherein the antisense strand comprises a DNA nucleotide at positions 2, 5, 7, 12, 14 and 16 counting from the 5′-end of the antisense strand.

Embodiment 77: The dsRNA of any one of the preceding Embodiments, wherein the antisense strand comprises a DNA nucleotide at positions 2, 5, 7, and 12 counting from the 5′-end of the antisense strand; and a 2′-fluoro nucleotide at position 14 of the antisense strand.

Embodiment 78: The dsRNA of any one of the preceding Embodiments, wherein the dsRNA comprises at least one, e.g., 2, 3, 4, 5, 6, 7, 8, 9, 10 or more 2′-OMe nucleotides.

Embodiment 79: The dsRNA of any one of the preceding Embodiments, wherein the antisense strand comprises at least one 2′-OMe nucleotide.

Embodiment 80: The dsRNA of any one of the preceding Embodiments, wherein all remaining nucleotides in the antisense strand are 2′-OMe nucleotides.

Embodiment 81: The dsRNA of any one of the preceding Embodiments, wherein the sense strand comprises at least one 2′-OMe nucleotide.

Embodiment 82: The dsRNA of anyone of the preceding Embodiments, wherein all remaining nucleotides in the sense strand are 2′-OMe nucleotides.

Embodiment 83: The dsRNA of any one of the preceding Embodiments, wherein the antisense strand comprises a phosphate group or a phosphate analog or derivative thereof at its 5′-end.

Embodiment 84: The dsRNA of any one of the preceding Embodiments, wherein the antisense strand comprises a vinylphosphonate (e.g., E-vinylphosphonate) group at its 5′-end.

Embodiment 85: The dsRNA of any one of the preceding Embodiments, wherein the dsRNA comprises at least one, e.g., 2, 3, 4, 5, 6, 7, 8, 9, 10 or more locked nucleic acid (LNA) or bridged nucleic acid (BNA) nucleotides.

Embodiment 86: The dsRNA of any one of the preceding Embodiments, wherein the antisense strand comprises at least one, e.g., 2, 3, 4, 5 or more LNA or BNA nucleotides.

Embodiment 87: The dsRNA of any one of the preceding Embodiments, wherein the sense strand comprises at least one, e.g., 2, 3, 4, 5 or more LNA or BNA nucleotides.

Embodiment 88: The dsRNA of any one of the preceding Embodiments, wherein the dsRNA comprises at least one, e.g., 2, 3, 4, 5, 6, 7, 8, 9, 10 or more cyclohexene nucleic acid (CeNA) nucleotides.

Embodiment 89: The dsRNA of any one of the preceding Embodiments, wherein the antisense strand comprises at least one, e.g., 2, 3, 4, 5 or more CeNA nucleotides.

Embodiment 90: The dsRNA of any one of the preceding Embodiments, wherein the sense strand comprises at least one, e.g., 2, 3, 4, 5 or more CeNA nucleotides.

Embodiment 91: The dsRNA of any one of the preceding Embodiments, wherein the dsRNA comprises at least one, e.g., 2, 3, 4, 5, 6, 7, 8, 9, 10 or more thermally stabilizing modifications.

Embodiment 92: The dsRNA of any one of the preceding Embodiments, wherein the antisense strand comprises at least one, e.g., 2, 3, 4, 5 or more thermally stabilizing modifications.

Embodiment 93: The dsRNA of any one of the preceding Embodiments, wherein the sense strand comprises at least one, e.g., 2, 3, 4, 5 or more thermally stabilizing modifications.

Embodiment 94: The dsRNA of any one of the preceding Embodiments, wherein the dsRNA comprises at least one, e.g., 2, 3, 4, 5, 6, 7, 8, 9, 10 or more abasic nucleotides.

Embodiment 95: The dsRNA of any one of the preceding Embodiments, wherein the antisense strand comprises at least one, e.g., 2, 3, 4, 5 or more abasic nucleotides.

Embodiment 96: The dsRNA of any one of the preceding Embodiments, wherein the sense strand comprises at least one, e.g., 2, 3, 4, 5 or more abasic nucleotides.

Embodiment 97: The dsRNA of any one of the preceding Embodiments, wherein the dsRNA comprises at least one, e.g., 2, 3, 4, 5, 6, 7, 8, 9, 10 or more 2′-deoxy nucleotides.

Embodiment 98: The dsRNA of any one of the preceding Embodiments, wherein the antisense strand comprises at least one, e.g., 2, 3, 4, 5 or more 2′-deoxy nucleotides.

Embodiment 99: The dsRNA of any one of the preceding Embodiments, wherein the sense strand comprises at least one, e.g., 2, 3, 4, 5 or more 2′-deoxy nucleotides.

Embodiment 100: The dsRNA of any one of the preceding Embodiments, wherein the dsRNA comprises at least one, e.g., 2, 3, 4, 5, 6, 7, 8, 9, 10 or more acyclic (e.g., unlocked nucleic acid (UNA) or glycol nucleic acid (GNA)) nucleotides.

Embodiment 101: The dsRNA of any one of the preceding Embodiments, wherein the antisense strand comprises at least one, e.g., 2, 3, 4, 5 or more acyclic (e.g., unlocked nucleic acid (UNA) or glycol nucleic acid (GNA)) nucleotides.

Embodiment 102: The dsRNA of any one of the preceding Embodiments, wherein the sense strand comprises at least one, e.g., 2, 3, 4, 5 or more acyclic (e.g., unlocked nucleic acid (UNA) or glycol nucleic acid (GNA)) nucleotides.

Embodiment 103: The dsRNA of any one of the preceding Embodiments, wherein the dsRNA comprises at least one thermally destabilizing modification.

Embodiment 104: The dsRNA of any one of the preceding Embodiments, wherein the antisense strand comprises at least one thermally destabilizing modification.

Embodiment 105: The dsRNA of any one of the preceding Embodiments, wherein the antisense strand comprises at least one thermally destabilizing modification in the seed region (i.e., positions 2-9 from the 5′-end) of the antisense strand.

Embodiment 106: The dsRNA of any one of the preceding Embodiments, wherein the antisense strand comprises a thermally destabilizing modification at least at one of positions 6, 7 or 8, counting from the 5′-end of the strand.

Embodiment 107: The dsRNA of any one of the preceding Embodiments, wherein the antisense strand comprises a thermally destabilizing modification at position 7, counting from the 5′-end of the strand.

Embodiment 108: The dsRNA of any one of Embodiments 103-107, wherein the thermally destabilizing modification is an abasic nucleotide, 2′-deoxy nucleotides, acyclic nucleotide (e.g., unlocked nucleic acid (UNA), glycol nucleic acid (GNA) or(S)-glycol nucleic acid (S-GNA)), a 2′-5′ linked nucleotide (3′-RNA), threose nucleotide (TNA), 2′ gem Me/F nucleotide, or mismatch with an opposing nucleotide in the other strand.

Embodiment 109: The dsRNA of any one of the preceding Embodiments, wherein the first ligand is GalNAc and the second ligand is a mannose receptor targeting ligand (e.g., multivalent mannose).

Embodiment 110: The dsRNA of any one of the preceding Embodiments, wherein the first ligand is GalNAc and the second ligand is a folic acid ligand.

Embodiment 111: A compound of Formula (I):

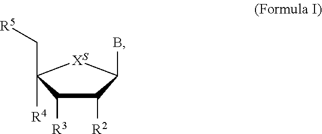

wherein:
B an optionally modified nucleobase;
X^{S} is O, CH₂, S, or NH;
R² is hydroxyl, protected hydroxyl, halogen, optionally substituted C₁₋₃₀ alkoxy (e.g., methoxy, 2-methoxyethoxy), alkoxyalkyl (e.g., 2-methoxyethyl), hydrogen, optionally substituted C₁₋₃₀ alkyl, optionally substituted C₂₋₃₀alkenyl, optionally substituted C₂₋₃₀alkynyl, alkoxyalkylamine, alkoxyoxycarboxylate, amino, alkylamino, dialkylamino, 5-8 membered heterocyclyl, —O—C₄₋₃₀alkyl-ON(CH₂R⁸) (CH₂R⁹), —O—C₄₋₃₀alkyl-ON(CH₂R⁸)(CH₂R⁹), phosphate group, reactive phosphorous group, a ligand, or a linker covalently bonded to one or more ligands;
R³ is a reactive phosphorous group, hydroxyl, protected hydroxyl, halogen, optionally substituted C₂₋₃₀alkynyl, optionally substituted C₁₋₃₀ alkoxy (e.g., methoxy, 2-methoxyethoxy), alkoxyalkyl (e.g., 2-methoxyethyl), hydrogen, optionally substituted C₁₋₃₀ alkyl, optionally substituted C₂₋₃₀alkenyl, alkoxyalkylamine, alkoxyoxycarboxylate, amino, alkylamino, dialkylamino, 5-8 membered heterocyclyl, —O—C₄₋₃₀alkyl-ON(CH₂R⁸)(CH₂R⁹), —O—C₄₋₃₀alkyl-ON(CH₂R⁸)(CH₂R⁹), phosphate group, a ligand, or a linker covalently bonded to one or more ligands;
R⁴ is hydrogen, optionally substituted C₁₋₆alkyl, optionally substituted C₂₋₆alkenyl, optionally substituted C₂₋₆alkynyl, or optionally substituted C₁₋₆alkoxy;
or R⁴ and R² taken together are 4′-C(R¹⁰R¹¹)ᵥ—Y-2′ or 4′-Y—C(R¹⁰R¹¹)ᵥ-2′;
Y is —O—, —CH₂—, —CH(Me)-, —C(CH₃)₂—, —S—, —N(R¹²)—, —C(O)—, —C(S)—, —S(O)—, —S(O)₂—, —OC(O)—, —C(O)O—, —N(R¹²)C(O)—, or —C(O)N(R¹²)—;
R¹⁰ and R¹¹ independently are H, optionally substituted C₁-C₆alkyl, optionally substituted C₂-C₆alkenyl or optionally substituted C₂-C₆alkynyl;
R¹² is hydrogen, optionally substituted C₁₋₃₀alkyl, optionally substituted C₁-C₃₀alkoxy, C₁₋₄haloalkyl, optionally substituted C₂₋₄alkenyl, optionally substituted C₂₋₄alkynyl, optionally substituted C₁₋₃₀alkyl-CO₂H, or a nitrogen-protecting group;
v is 1, 2 or 3; and
R⁵ is -L¹-R^{H}, —O—N(R¹³)R¹⁴;
L¹ is a bond, -L³-, C₁₋₃₀alkylene, C₂₋₃₀alkenylene, C₂₋₃₀alkynylene, *-L³-C₁₋₃₀alkylene *-L³-C₂₋₃₀alkenylene, or *-L³-C₂₋₃₀alkynylene;
L³ is —O—, —N(R^{L3})—, —S—, —C(O)—, —S(O)—, —S(O)₂—, —P(X^{L3})(Y^{L3}R^{L3B})—;
where R^{L3} is hydrogen, optionally substituted C₁₋₃₀alkyl, optionally substituted C₁-C₃₀alkoxy, C₁₋₄haloalkyl, optionally substituted C₂₋₄alkenyl, optionally substituted C₂₋₄alkynyl, optionally substituted C₁₋₃₀alkyl-CO₂H, or a nitrogen-protecting group;
X^{L2} is O or S;
Y^{L3} is O, S, NH, or a bond; and
R^{L3B} is H or optionally substituted alkyl; and
* is bond to R^{H};
and
R^{H} is 4-8 membered heterocyclyl comprising 1, 2 or 3 heteroatoms selected independently from N, O and S, and the heterocyclyl is optionally substituted with 1, 2, 3 or 4 independently selected substituents, and provided that the heterocyclyl comprises at least one nitrogen atom,
or R^{H} is

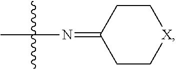

where X is O, NR^{L}, S, or CH₂; and R^{L} is hydrogen, a ligand, a linker covalently bonded to one or more ligands, aliphatic and aromatic alkyl, alkylester, alkylamine, dimethylamino alkyl, alkylether, alkylthioether, heteroaromatic alkyl, allyl, vinyl, alkyl groups functionalized with disulfide, oxime, ketone, acetal, hemiacetal, cleavable peptides, or cleavable sugars; and

R¹³ and R¹⁴ are independently -L²-R^{H2} or C₁-C₆alkyl, where:
L² is a linker; and
R^{H2} is 4-8 membered heterocyclyl comprising 1, 2 or 3 heteroatoms selected independently from N, O and S, and the heterocyclyl is optionally substituted with 1, 2, 3 or 4 independently selected substituents, and
provided that at least one of R¹³ and R¹⁴ is -L²-R^{H2}, and
provided that only one of R² and R³ is a reactive phosphorous group; and
R⁵ is not morpholin-4-yl unless R⁴ and R² taken together are 4′-C(R¹⁰R¹¹)ᵥ—Y-2′ or 4′-Y—C(R¹⁰R¹¹)ᵥ-2′.

Embodiment 112: The compound of Embodiment 111, wherein R⁵ is -L¹-R^{H}.

Embodiment 113: The compound of Embodiment 112, wherein L¹ is L³ or C₁₋₃₀alkylene.

Embodiment 114: The compound of Embodiment 112, wherein L¹ is O.

Embodiment 115: The compound of Embodiment 112, wherein L¹ is —(CH₂)ₙ—, where n is 0 or an integer selected from 1 to 30 (e.g., n is 1, 2, 3, 4, 5, 6, 7, 8, 9, 10, 11, 12, 13, 14, 15, 16, 16, 17, 18, 19, 20, 21, 22, 23, 24, 25, 26, 27, 28, 29 or 30, such as n is 1, 2, 3, 4, 5 or 6, preferably n is 0 or 1).

Embodiment 116: The compound of any one of Embodiments 112-115, wherein R^{H} is an optionally substituted 6-membered heterocyclyl comprising a nitrogen atom and 0, 1 or 2 additional heteroatoms selected independently from N, O and S.

Embodiment 117: The compound of any one of Embodiments 112-116, wherein R^{H} is

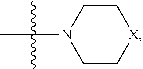

where X is O, NR^{L}, S, or CH₂; and R^{L} is hydrogen, a ligand, a linker covalently bonded to one or more ligands, aliphatic and aromatic alkyl, alkylester, alkylamine, dimethylamino alkyl, alkylether, alkylthioether, heteroaromatic alkyl, allyl, vinyl, alkyl groups functionalized with disulfide, oxime, ketone, acetal, hemiacetal, cleavable peptides, or cleavable sugars.

Embodiment 118: The compound of Embodiment 117, wherein X is O.

Embodiment 119: The compound of Embodiment 117, wherein X is NR^{L}.

Embodiment 120: The compound of Embodiment 119, wherein R^{L} is hydrogen.

Embodiment 121: The compound of Embodiment 119, wherein R^{L} is a ligand or linker covalently bonded to one or more independently selected ligands.

Embodiment 122: The compound of Embodiment 119, wherein R^{L} is aliphatic and aromatic alkyl, alkylester, alkylamine, dimethylamino alkyl, alkylether, alkylthioether, heteroaromatic alkyl, allyl, vinyl, alkyl groups functionalized with disulfide, oxime, ketone, acetal, hemiacetal, cleavable peptides, or cleavable sugars.

Embodiment 123: The compound of any one of Embodiments 111-114, wherein R^{H} is

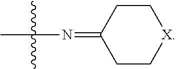

Embodiment 124: The compound of Embodiment 123, wherein X is O.

Embodiment 125: The compound of Embodiment 123, wherein X is NR^{L}.

Embodiment 126: The compound of Embodiment 123, wherein R^{L} is hydrogen.

Embodiment 127: The compound of Embodiment 123, wherein R^{L} is a ligand or linker covalently bonded to one or more independently selected ligands.

Embodiment 128: The compound of Embodiment 123, wherein R^{L} is aliphatic and aromatic alkyl, alkylester, alkylamine, dimethylamino alkyl, alkylether, alkylthioether, heteroaromatic alkyl, allyl, vinyl, alkyl groups functionalized with disulfide, oxime, ketone, acetal, hemiacetal, cleavable peptides, or cleavable sugars.

Embodiment 129: The compound of Embodiment 111, wherein R⁵ is —O—N(R¹³)R¹⁴.

Embodiment 130: The compound of Embodiment 129, wherein R¹³ and R¹⁴ are same.

Embodiment 131: The compound of Embodiment 129, wherein R¹³ and R¹⁴ are different.

Embodiment 132: The compound of any one of Embodiments 129-131, wherein one of R¹³ and R¹⁴ is -L²-R^{H2}.

Embodiment 133: The compound of any one of Embodiments 129-132, wherein L² is a bond or an optionally substituted alkylene.

Embodiment 134: The compound of Embodiment 133, wherein L² is a bond.

Embodiment 135: The compound of Embodiment 133, wherein L² is —Z—(CH₂)ₘ—, where Z is absent, aryl, heteroaryl, cycloalkyl or heterocyclyl; and m is 0 or an integer selected from 1 to 20 (e.g., m is 1, 2, 3, 4, 5, 6, 7, 8, 9, 10, 11, 12, 13, 14 or 15, such as m is 1, 2, 3, 4, 5 or 6).

Embodiment 136: The compound of any one of Embodiments 129-135, wherein one of R¹³ and R¹⁴ is —(CH₂)ₘ—R^{H2} or

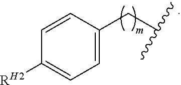

Embodiment 137: The compound of any one of Embodiments 129-136, wherein R^{H2} is an optionally substituted 6-membered heterocyclyl comprising a nitrogen atom and 0, 1 or 2 additional heteroatoms selected independently from N, O and S.

Embodiment 138: The compound of Embodiment 137, wherein R^{H2}

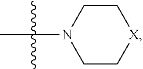

where X is O, NR^{L}, S, or CH₂; and R^{L} is hydrogen, a ligand, a linker covalently bonded to one or more ligands, aliphatic and aromatic alkyl, alkylester, alkylamine, dimethylamino alkyl, alkylether, alkylthioether, heteroaromatic alkyl, allyl, vinyl, alkyl groups functionalized with disulfide, oxime, ketone, acetal, hemiacetal, cleavable peptides, or cleavable sugars.

Embodiment 139: The compound of Embodiment 138, wherein X is O.

Embodiment 140: The compound of Embodiment 138, wherein X is NR^{L}.

Embodiment 141: The compound of Embodiment 140, wherein R^{L} is hydrogen.

Embodiment 142: The compound of Embodiment 140, wherein R^{L} is a ligand or linker covalently bonded to one or more independently selected ligands.

Embodiment 143: The compound of Embodiment 140, wherein R^{L} is aliphatic and aromatic alkyl, alkylester, alkylamine, dimethylamino alkyl, alkylether, alkylthioether, heteroaromatic alkyl, allyl, vinyl, alkyl groups functionalized with disulfide, oxime, ketone, acetal, hemiacetal, cleavable peptides, or cleavable sugars.

Embodiment 144: The compound of any one of Embodiments 129-143, wherein R^{H2} is

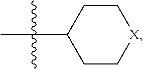

where X is O, NR^{L}, S, or CH₂; and R^{L} is hydrogen, a ligand, a linker covalently bonded to one or more ligands, aliphatic and aromatic alkyl, alkylester, alkylamine, dimethylamino alkyl, alkylether, alkylthioether, heteroaromatic alkyl, allyl, vinyl, alkyl groups functionalized with disulfide, oxime, ketone, acetal, hemiacetal, cleavable peptides, or cleavable sugars.

Embodiment 145: The compound of Embodiment 144, wherein X is O.

Embodiment 146: The compound of Embodiment 144, wherein X is NR^{L}.

Embodiment 147: The compound of Embodiment 146, wherein R^{L} is hydrogen.

Embodiment 148: The compound of Embodiment 146, wherein R^{L} is a ligand or linker covalently bonded to one or more independently selected ligands.

Embodiment 149: The compound of Embodiment 146, wherein R^{L} is aliphatic and aromatic alkyl, alkylester, alkylamine, dimethylamino alkyl, alkylether, alkylthioether, heteroaromatic alkyl, allyl, vinyl, alkyl groups functionalized with disulfide, oxime, ketone, acetal, hemiacetal, cleavable peptides, or cleavable sugars.

Embodiment 150: The compound of any one of Embodiments 129-149, wherein one of R¹³ and R¹⁴ is an optionally substituted C₁-C₆alkyl.

Embodiment 151: The compound of Embodiment 150, wherein one of R¹³ and R¹⁴ is methyl.

Embodiment 152: The compound of any one of Embodiments 111-151, wherein X^{S} is O or CH₂.

Embodiment 153: The compound of any one of Embodiments 111-152, wherein X^{S} is O.

Embodiment 154: The compound of any one of Embodiments 111-153, wherein R³ is a reactive phosphorous group, hydroxyl, or protected hydroxyl.

Embodiment 155: The compound of Embodiment 154, wherein R³ is a reactive phosphorous group.

Embodiment 156: The compound of any one of Embodiments 154-155, wherein R² is hydroxyl, protected hydroxyl, halogen, optionally substituted C₁₋₃₀ alkoxy (e.g., methoxy, 2-methoxyethoxy), alkoxyalkyl (e.g., 2-methoxyethyl), hydrogen, optionally substituted C₁₋₃₀ alkyl, optionally substituted C₂₋₃₀alkenyl, optionally substituted C₂₋₃₀alkynyl, alkoxyalkylamine, alkoxyoxycarboxylate, amino, alkylamino, dialkylamino, 5-8 membered heterocyclyl, —O—C₄₋₃₀alkyl-ON(CH₂R⁸)(CH₂R⁹), or —O—C₄₋₃₀alkyl-ON(CH₂R⁸)(CH₂R⁹); or R² and R⁴ taken together are 4′-C(R¹⁰R¹¹)ᵥ—Y-2′ or 4′-Y—C(R¹⁰R¹¹)ᵥ-2′.

Embodiment 157: The compound of any one of Embodiments 154-156, wherein R² is hydroxyl, protected hydroxyl, halogen, optionally substituted C₁₋₃₀ alkoxy (e.g., methoxy, 2-methoxyethoxy), alkoxyalkyl (e.g., 2-methoxyethyl), hydrogen, amino, alkylamino, or dialkylamino; or R² and R⁴ taken together are 4′-C(R¹⁰R¹¹)ᵥ—Y-2′ or 4′-Y—C(R¹⁰R¹¹)ᵥ-2′.

Embodiment 158: The compound of any one of Embodiments 154-157, wherein R² is hydrogen, hydroxyl, protected hydroxyl, fluoro, methoxy, ethoxy, or 2-methoxyethoxy; or R² and R⁴ taken together are 4′-C(R¹⁰R¹¹)ᵥ—Y-2′ or 4′-Y—C(R¹⁰R¹¹)ᵥ-2′.

Embodiment 159: The compound of any one of Embodiments 154-158, wherein R² is hydrogen, hydroxyl, protected hydroxyl, fluoro, or methoxy.

Embodiment 160: The compound of any one of Embodiments 154-159, wherein R² and R⁴ taken together are 4′-C(R¹⁰R¹¹)ᵥ—Y-2′ or 4′-Y—C(R¹⁰R¹¹)ᵥ-2′.

Embodiment 161: The compound of Embodiment 160, wherein R² and R⁴ taken together are 4′-C(R¹⁰R¹¹)ᵥ—Y-2′, wherein v is 1 or 2.

Embodiment 162: The compound of Embodiment 160 or 161, wherein one of R¹⁰ and R¹¹ is H and the other is independently H or optionally substituted C₁-C₆alkyl.

Embodiment 163: The compound of Embodiment 162 wherein R² and R⁴ taken together are 4′-CH₂—O-2′.

Embodiment 164: The compound of any one of Embodiments 154-159, wherein R⁴ is H.

Embodiment 165: The compound of any one of Embodiments 111-153, wherein R² is a reactive phosphorous group, hydroxyl, or protected hydroxyl.

Embodiment 166: The compound of Embodiment 165, wherein R² is a reactive phosphorous group.

Embodiment 167: The compound of any one of Embodiments 165-166, wherein R³ is hydroxyl, protected hydroxyl, halogen, optionally substituted C₁₋₃₀ alkoxy (e.g., methoxy, 2-methoxyethoxy), alkoxyalkyl (e.g., 2-methoxyethyl), hydrogen, optionally substituted C₁₋₃₀ alkyl, optionally substituted C₂₋₃₀alkenyl, optionally substituted C₂₋₃₀alkynyl, alkoxyalkylamine, alkoxyoxycarboxylate, amino, alkylamino, dialkylamino, 5-8 membered heterocyclyl, —O—C₄₋₃₀alkyl-ON(CH₂R⁸) (CH₂R⁹), or —O—C₄₋₃₀alkyl-ON(CH₂R⁸)(CH₂R⁹).

Embodiment 168: The compound of any one of Embodiments 165-167, wherein R³ is hydroxyl, protected hydroxyl, halogen, optionally substituted C₁₋₃₀ alkoxy (e.g., methoxy, 2-methoxyethoxy), alkoxyalkyl (e.g., 2-methoxyethyl), hydrogen, amino, alkylamino, or dialkylamino.

Embodiment 169: The compound of any one of Embodiments 165-168, wherein R³ is hydrogen, hydroxyl, protected hydroxyl, fluoro, methoxy, ethoxy, or 2-methoxyethoxy; or R² and R⁴ taken together are 4′-C(R¹⁰R¹¹)ᵥ—Y-2′ or 4′-Y—C(R¹⁰R¹¹)ᵥ-2′.

Embodiment 170: The compound of any one of Embodiments 165-169, wherein R³ is hydrogen, hydroxyl, protected hydroxyl, fluoro, or methoxy.

Embodiment 171: The compound of any one of Embodiments 165-170, wherein R⁴ is H.

Embodiment 172: The compound of Embodiment 111, wherein compound is selected from formulae (I-A)-(I-D):

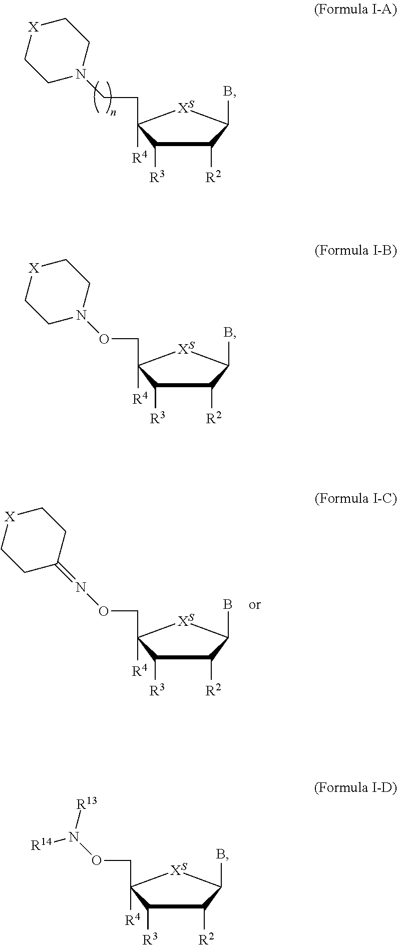

wherein:
n is 0 or an integer selected from 1 to 30 (e.g., n is 1, 2, 3, 4, 5, 6, 7, 8, 9, 10, 11, 12, 13, 14, 15, 16, 16, 17, 18, 19, 20, 21, 22, 23, 24, 25, 26, 27, 28, 29 or 30, such as n is 1, 2, 3, 4, 5 or 6, preferably n is 0 or 1);
R² is hydrogen, hydroxyl, protected hydroxyl, halogen, optionally substituted C₁₋₃₀ alkoxy (e.g., methoxy, 2-methoxyethoxy), alkoxyalkyl (e.g., 2-methoxyethyl), amino, alkylamino, or dialkylamino;
R³ is a reactive phosphorous group, hydroxyl, protected hydroxyl or a reactive phosphorous group;
R⁴ is hydrogen or R² and R⁴ taken together are 4′-C(R¹⁰R¹¹)ᵥ—Y-2′ or 4′-Y—C(R¹⁰R¹¹)ᵥ-2′.

Embodiment 174: The compound of Embodiment 172 or 173, wherein R³ is a reactive phosphorous group (e.g., a phosphoramidite, such as 3′-[(2-cyanoethyl)-(N,N-diisopropyl)]-phosphoramidite, 3′-[(2-cyanoethyl)-(N,N-diisopropyl)]-phosphoramidite, or 3′-[(ß-thiobenzoylethyl)-(1-pyrrolidinyl)]-thiophosphoramidite).

Embodiment 173: The compound of Embodiment 172, wherein X^{S} is O.

Embodiment 175: The compound of Embodiment 172 or 173, wherein R³ is hydroxyl or protected hydroxyl.

Embodiment 176: The compound of any one of Embodiments 172-175, wherein R² is hydrogen, hydroxyl, protected hydroxyl, halogen, optionally substituted C₁₋₃₀ alkoxy (e.g., methoxy, 2-methoxyethoxy), or alkoxyalkyl (e.g., 2-methoxyethyl).

Embodiment 177: The compound of any one of Embodiments 172-176, wherein R² is hydrogen, hydroxyl, protected hydroxyl, fluoro, or methoxy.

Embodiment 178: The compound of any one of Embodiments 172-177, wherein R⁴ is H.

Embodiment 179: The compound of any one of Embodiments 172-175, wherein R² and R⁴ taken together are 4′-C(R¹⁰R¹¹)ᵥ—Y-2′ or 4′-Y—C(R¹⁰R¹¹)ᵥ-2′.

Embodiment 180: The compound of Embodiment 179, wherein one of R¹⁰ and R¹¹ is H and the other is independently H or optionally substituted C₁-C₆alkyl.

Embodiment 181: The compound of Embodiment 180, wherein R² and R⁴ taken together are 4′-CH₂—O-2′.

Embodiment 182: The compound of any one of Embodiments 172-181, wherein one of R¹³ and R¹⁴ is

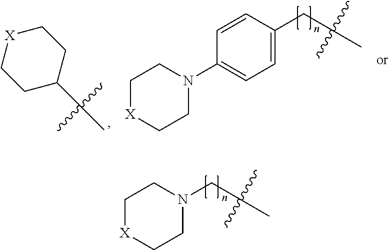

and the other of R¹³ and R¹⁴ is C₁-C₆alkyl,

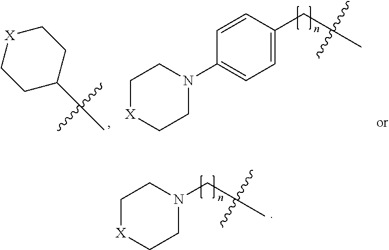

Embodiment 183: The compound of any one of Embodiments 172-182, wherein R¹³ and R¹⁴ are the same.

Embodiment 184: The compound of any one of Embodiments 172-182, wherein R¹³ and R¹⁴ are different.

Embodiment 185: The compound of Embodiment 184, wherein one of R¹³ and R¹⁴ is C₁-Calkyl (e.g., methyl).

Embodiment 186: The compound of any one of Embodiments 172-185, wherein X is NR^{L}.

Embodiment 187: The compound of Embodiment 186, wherein R^{L} is a ligand or linker covalently bonded to one or more independently selected ligands.

Embodiment 188: The compound of Embodiment 186, wherein R^{L} is aliphatic and aromatic alkyl, alkylester, alkylamine, dimethylamino alkyl, alkylether, alkylthioether, heteroaromatic alkyl, allyl, vinyl, alkyl groups functionalized with disulfide, oxime, ketone, acetal, hemiacetal, cleavable peptides, or cleavable sugars.

Embodiment 189: The compound of any one of Embodiment 172-185, wherein X is O, S, CH₂ or NH.

Embodiment 190: The compound of Embodiment 111, wherein the compound is of Formula (I-E):

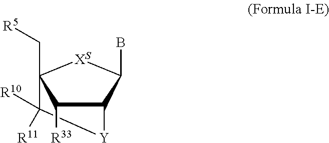

wherein:
R³ is a reactive phosphorous group, hydroxyl, or protected hydroxyl;
R⁵ is -L¹-R^{H}; and
X^{S}, B, Y, R¹⁰ and R¹¹ are as defined in claim 111.

Embodiment 191: The compound of Embodiment 190, wherein compound is of Formula (I-Ea), (I-E¹) or (I-E²):

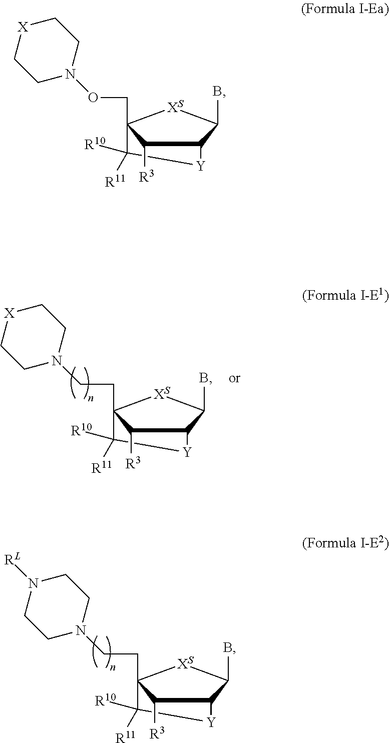

wherein
n is 0 or an integer selected from 1 to 30 (e.g., n is 1, 2, 3, 4, 5, 6, 7, 8, 9, 10, 11, 12, 13, 14, 15, 16, 16, 17, 18, 19, 20, 21, 22, 23, 24, 25, 26, 27, 28, 29 or 30, such as n is 1, 2, 3, 4, 5 or 6, preferably n is 0 or 1);
X is O, NR^{L}, S, or CH₂; and
R^{L} is H, a ligand, a linker covalently bonded to one or more ligands, aliphatic and aromatic alkyl, alkylester, alkylamine, dimethylamino alkyl, alkylether, alkylthioether, heteroaromatic alkyl, allyl, vinyl, alkyl groups functionalized with disulfide, oxime, ketone, acetal, hemiacetal, cleavable peptides, or cleavable sugars, and optionally, the compound is of Formula (I-Eb) or (I-Ec):

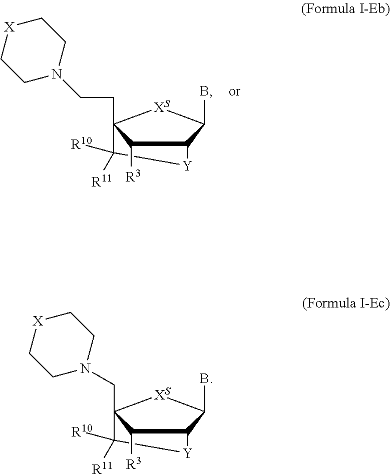

Embodiment 192: The compound of Embodiment 191, wherein X is O or CH₂.

Embodiment 193: The compound of Embodiment 191 or 192, wherein X is O.

Embodiment 194: The compound of any one of Embodiments 190-193, wherein Y is O.

Embodiment 195: The compound of any one of Embodiments 190-194, wherein one of R¹⁰ is H and the other is H or C₁₋₆alkyl (e.g., methyl).

Embodiment 196: The compound of any one of Embodiments 190-195, wherein X^{S} is O.

Embodiment 197: The compound of any one of Embodiments 190-196, wherein R³ is a reactive phosphorous group (e.g., a phosphoramidite, such as 3′-[(2-cyanoethyl)-(N,N-diisopropyl)]-phosphoramidite, 3′-[(2-cyanoethyl)-(N,N-diisopropyl)]-phosphoramidite, or 3′-[(β-thiobenzoylethyl)-(1-pyrrolidinyl)]-thiophosphoramidite).

Embodiment 198: The compound of any one of Embodiments 190-197, wherein R³ is hydroxyl or protected hydroxyl.

Embodiment 199: The compound of Embodiment 190, wherein the compound is of Formula (I-Ed), (I-Ee), (I-E³) or (I-E⁴):

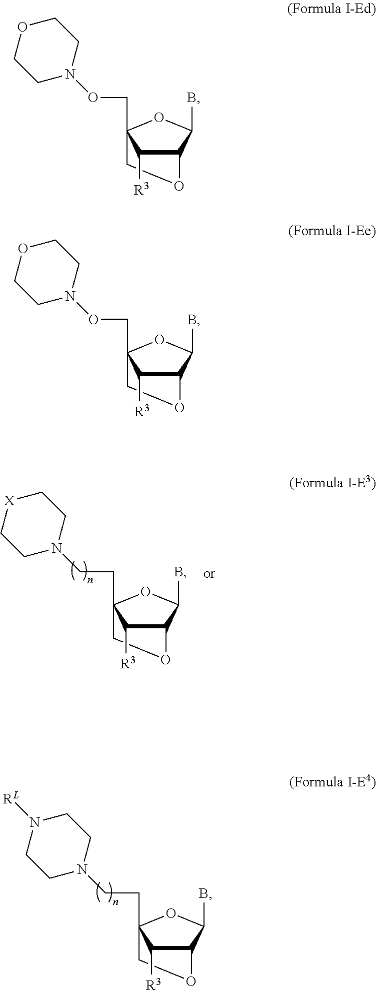

where n is 0 or an integer selected from 1 to 30 (e.g., n is 1, 2, 3, 4, 5, 6, 7, 8, 9, 10, 11, 12, 13, 14, 15, 16, 16, 17, 18, 19, 20, 21, 22, 23, 24, 25, 26, 27, 28, 29 or 30, such as n is 1, 2, 3, 4, 5 or 6, preferably n is 0 or 1); and
R^{L} is H, a ligand, a linker covalently bonded to one or more ligands, aliphatic and aromatic alkyl, alkylester, alkylamine, dimethylamino alkyl, alkylether, alkylthioether, heteroaromatic alkyl, allyl, vinyl, alkyl groups functionalized with disulfide, oxime, ketone, acetal, hemiacetal, cleavable peptides, or cleavable sugars, and optionally, the compound is of Formula (I-Ef) or (I-Eg):

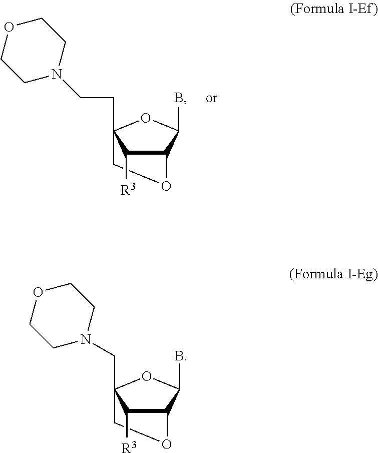

Embodiment 200: An oligonucleotide prepared using a compound of any one of Embodiments 111-199.

Embodiment 201: An oligonucleotide comprising at least one nucleoside of Formula (II):

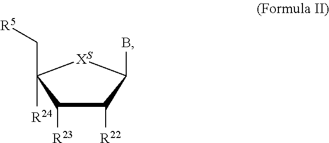

wherein:
B an optionally modified nucleobase;
X^{S} is O, CH₂, S, or NH;
R²² is hydroxyl, protected hydroxyl, halogen, optionally substituted C₁₋₃₀ alkoxy (e.g., methoxy, 2-methoxyethoxy), alkoxyalkyl (e.g., 2-methoxyethyl), hydrogen, optionally substituted C₁₋₃₀ alkyl, optionally substituted C₂₋₃₀alkenyl, optionally substituted C₂₋₃₀alkynyl, alkoxyalkylamine, alkoxyoxycarboxylate, amino, alkylamino, dialkylamino, 5-8 membered heterocyclyl, —O—C₄₋₃₀alkyl-ON(CH₂R⁸) (CH₂R⁹), —O—C₄₋₃₀alkyl-ON(CH₂R⁸) (CH₂R⁹), a ligand, a linker covalently bonded to one or more ligands or a bond to an internucleotide linkage to a subsequent nucleoside;
R²³ is a bond to an internucleotide linkage to a subsequent nucleoside, hydroxyl, protected hydroxyl, halogen, optionally substituted C₂₋₃₀alkynyl, optionally substituted C₁₋₃₀ alkoxy (e.g., methoxy, 2-methoxyethoxy), alkoxyalkyl (e.g., 2-methoxyethyl), hydrogen, optionally substituted C₁₋₃₀ alkyl, optionally substituted C₂₋₃₀alkenyl, alkoxyalkylamine, alkoxyoxycarboxylate, amino, alkylamino, dialkylamino, 5-8 membered heterocyclyl, —O—C₄₋₃₀alkyl-ON(CH₂R⁸) (CH₂R⁹), —O—C₄₋₃₀alkyl-ON(CH₂R⁸) (CH₂R⁹), phosphate group, a ligand, or a linker covalently bonded to one or more ligands;
R²⁴ is hydrogen, optionally substituted C₁₋₆alkyl, optionally substituted C₂₋₆alkenyl, optionally substituted C₂₋₆alkynyl, or optionally substituted C₁₋₆alkoxy;
or R²² and R²⁴ taken together are 4′-C(R¹⁰R¹¹)ᵥ—Y-2′ or 4′-Y—C(R¹⁰R¹¹)ᵥ-2′;
Y is —O—, —CH₂—, —CH(Me)-, —C(CH₃)₂—, —S—, —N(R¹²)—, —C(O)—, —C(S)—, —S(O)—, —S(O)₂—, —OC(O)—, —C(O)O—, —N(R¹²)C(O)—, or —C(O)N(R¹²)—;
R¹⁰ and R¹¹ independently are H, optionally substituted C₁-C₆alkyl, optionally substituted C₂-C₆alkenyl or optionally substituted C₂-C₆alkynyl;
R¹² is hydrogen, optionally substituted C₁₋₃₀alkyl, optionally substituted C₁-C₃₀alkoxy, C₁₄haloalkyl, optionally substituted C₂₋₄alkenyl, optionally substituted C₂₋₄alkynyl, optionally substituted C₁₋₃₀alkyl-CO₂H, or a nitrogen-protecting group;
v is 1, 2 or 3; and
R⁵ is -L¹-R^{H}, —O—N(R¹³)R¹⁴;
L¹ is a bond, -L³-, C₁₋₃₀alkylene, C₂₋₃₀alkenylene, C₂₋₃₀alkynylene, *-L³-C₁₋₃₀alkylene *-L³-C₂₋₃₀alkenylene, or *-L³-C₂₋₃₀alkynylene;
L³ is —O—, —N(R^{L3})—, —S—, —C(O)—, —S(O)—, —S(O)₂—, —P(X^{L3}) (Y^{L3}R^{L3B})—;
where R^{L3} is hydrogen, optionally substituted C₁₋₃₀alkyl, optionally substituted C₁-C₃₀alkoxy, C₁₄haloalkyl, optionally substituted C₂₋₄alkenyl, optionally substituted C₂₋₄alkynyl, optionally substituted C₁₋₃₀alkyl-CO₂H, or a nitrogen-protecting group;
X^{L2} is O or S;
Y^{L3} is O, S, NH, or a bond; and
R^{L3B} is H or optionally substituted alkyl; and
* is bond to R^{H};
and
R^{H} is 4-8 membered heterocyclyl comprising 1, 2 or 3 heteroatoms selected independently from N, O and S, and the heterocyclyl is optionally substituted with 1, 2, 3 or 4 independently selected substituents, and provided that the heterocyclyl comprises at least one nitrogen atom, or R^{H} is

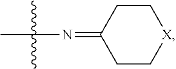

where X is O, NR^{L}, S, or CH₂; and R^{L} is hydrogen, a ligand, a linker covalently bonded to one or more ligands, aliphatic and aromatic alkyl, alkylester, alkylamine, dimethylamino alkyl, alkylether, alkylthioether, heteroaromatic alkyl, allyl, vinyl, alkyl groups functionalized with disulfide, oxime, ketone, acetal, hemiacetal, cleavable peptides, or cleavable sugars; and
R¹³ and R¹⁴ are independently -L²-R^{H2}, where:
L² is a linker; and
R^{H2} is 4-8 membered heterocyclyl comprising 1, 2 or 3 heteroatoms selected independently from N, O and S, and the heterocyclyl is optionally substituted with 1, 2, 3 or 4 independently selected substituents, and
provided that at least one of R¹³ and R¹⁴ is -L²-R^{H2}, and
provided that one of R²² and R²³ is a bond to an internucleotide linkage to a subsequent nucleoside and only one of R²² and R²³ is a bond to an internucleotide linkage to a subsequent nucleoside.

Embodiment 202: The oligonucleotide of Embodiment 201, wherein R⁵ is -L¹-R^{H}.

Embodiment 203: The oligonucleotide of Embodiment 202, wherein L¹ is -L³- or C₁₋₃₀alkylene.

Embodiment 204: The oligonucleotide of Embodiment 202, wherein L¹ is O.

Embodiment 205: The oligonucleotide of Embodiment 202, wherein L¹ is —(CH₂)ₙ—, where n is 0 or an integer selected from 1 to 30 (e.g., n is 1, 2, 3, 4, 5, 6, 7, 8, 9, 10, 11, 12, 13, 14, 15, 16, 16, 17, 18, 19, 20, 21, 22, 23, 24, 25, 26, 27, 28, 29 or 30, such as n is 1, 2, 3, 4, 5 or 6, preferably n is 0 or 1).

Embodiment 206: The oligonucleotide of any one of Embodiments 202-205, wherein R^{H} is an optionally substituted 6-membered heterocyclyl comprising a nitrogen atom and 0, 1 or 2 additional heteroatoms selected independently from N, O and S.

Embodiment 207: The oligonucleotide of any one of Embodiments 202-206, wherein R^{H} is

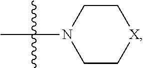

where X is O, NR^{L}, S, or CH₂; and R^{L} is hydrogen, a ligand, a linker covalently bonded to one or more ligands, aliphatic and aromatic alkyl, alkylester, alkylamine, dimethylamino alkyl, alkylether, alkylthioether, heteroaromatic alkyl, allyl, vinyl, alkyl groups functionalized with disulfide, oxime, ketone, acetal, hemiacetal, cleavable peptides, or cleavable sugars.

Embodiment 208: The oligonucleotide of Embodiment 207, wherein X is O.

Embodiment 209: The oligonucleotide of Embodiment 207, wherein X is NR^{L}.

Embodiment 210: The oligonucleotide of Embodiment 209, wherein R^{L} is hydrogen.

Embodiment 211: The oligonucleotide of Embodiment 209, wherein R^{L} is a ligand or linker covalently bonded to one or more independently selected ligands.

Embodiment 212: The oligonucleotide of Embodiment 209, wherein R^{L} is aliphatic and aromatic alkyl, alkylester, alkylamine, dimethylamino alkyl, alkylether, alkylthioether, heteroaromatic alkyl, allyl, vinyl, alkyl groups functionalized with disulfide, oxime, ketone, acetal, hemiacetal, cleavable peptides, or cleavable sugars.

Embodiment 213: The oligonucleotide of any one of Embodiments 201-205, wherein R^{H} is

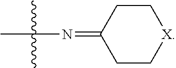

Embodiment 214: The oligonucleotide of Embodiment 213, wherein X is O.

Embodiment 215: The oligonucleotide of Embodiment 213, wherein X is NR^{L}.

Embodiment 216: The oligonucleotide of Embodiment 215, wherein R^{L} is hydrogen.

Embodiment 217: The oligonucleotide of Embodiment 215, wherein R^{L} is a ligand or linker covalently bonded to one or more independently selected ligands.

Embodiment 218: The oligonucleotide of Embodiment 215, wherein R^{L} is aliphatic and aromatic alkyl, alkylester, alkylamine, dimethylamino alkyl, alkylether, alkylthioether, heteroaromatic alkyl, allyl, vinyl, alkyl groups functionalized with disulfide, oxime, ketone, acetal, hemiacetal, cleavable peptides, or cleavable sugars.

Embodiment 219: The oligonucleotide of Embodiment 201, wherein R⁵ is —O—N(R¹³)R¹⁴

Embodiment 220: The oligonucleotide of Embodiment 219, wherein R¹³ and R¹⁴ are same.

Embodiment 221: The oligonucleotide of Embodiment 219, wherein R¹³ and R¹⁴ are different.

Embodiment 222: The oligonucleotide of any one of Embodiments 219-221, wherein one of R¹³ and R¹⁴ is -L²-R^{H2}.

Embodiment 223: The oligonucleotide of any one of Embodiments 219-222, wherein L² is a bond or an optionally substituted alkylene.

Embodiment 224: The oligonucleotide of Embodiment 223, wherein L² is a bond.

Embodiment 225: The oligonucleotide of Embodiment 223, wherein L² comprises —Z—(CH₂)ₘ—, where Z is absent, aryl, heteroaryl, cycloalkyl or heterocyclyl; and m is 0 or an integer selected from 1 to 20 (e.g., m is 1, 2, 3, 4, 5, 6, 7, 8, 9, 10, 11, 12, 13, 14 or 15, such as m is 1, 2, 3, 4, 5 or 6).

Embodiment 226: The oligonucleotide of any one of Embodiments 219-225, wherein one of R¹³ and R¹⁴ is —(CH₂)ₘ—R^{H2} or

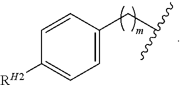

Embodiment 227: The oligonucleotide of any one of Embodiments 219-226, wherein R^{H2} is an optionally substituted 6-membered heterocyclyl comprising a nitrogen atom and 0, 1 or 2 additional heteroatoms selected independently from N, O and S.

Embodiment 228: The oligonucleotide of Embodiment 227, wherein R^{H2} is

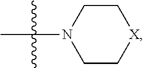

where X is O, NR^{L}, S, or CH₂; and R^{L} is hydrogen, a ligand, a linker covalently bonded to one or more ligands, aliphatic and aromatic alkyl, alkylester, alkylamine, dimethylamino alkyl, alkylether, alkylthioether, heteroaromatic alkyl, allyl, vinyl, alkyl groups functionalized with disulfide, oxime, ketone, acetal, hemiacetal, cleavable peptides, or cleavable sugars.

Embodiment 229: The oligonucleotide of Embodiment 228, wherein X is O.

Embodiment 230: The oligonucleotide of Embodiment 228 wherein X is NR^{L}.

Embodiment 231: The oligonucleotide of Embodiment 230, wherein R^{L} is hydrogen.

Embodiment 232: The oligonucleotide of Embodiment 230, wherein R^{L} is a ligand or linker covalently bonded to one or more independently selected ligands.

Embodiment 233: The oligonucleotide of Embodiment 230, wherein R^{L} is aliphatic and aromatic alkyl, alkylester, alkylamine, dimethylamino alkyl, alkylether, alkylthioether, heteroaromatic alkyl, allyl, vinyl, alkyl groups functionalized with disulfide, oxime, ketone, acetal, hemiacetal, cleavable peptides, or cleavable sugars.

Embodiment 234: The oligonucleotide of any one of Embodiments 219-233, wherein R^{H2} is

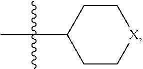

where X is O, NR^{L}, S, or CH₂; and R^{L} is hydrogen, a ligand, a linker covalently bonded to one or more ligands, aliphatic and aromatic alkyl, alkylester, alkylamine, dimethylamino alkyl, alkylether, alkylthioether, heteroaromatic alkyl, allyl, vinyl, alkyl groups functionalized with disulfide, oxime, ketone, acetal, hemiacetal, cleavable peptides, or cleavable sugars.

Embodiment 235: The oligonucleotide of Embodiment 234, wherein X is O.

Embodiment 236: The oligonucleotide of Embodiment 234, wherein X is NR^{L}.

Embodiment 237: The oligonucleotide of Embodiment 236, wherein R^{L} is hydrogen.

Embodiment 238: The oligonucleotide of Embodiment 236, wherein R^{L} is a ligand or linker covalently bonded to one or more independently selected ligands.

Embodiment 239: The oligonucleotide of Embodiment 236, wherein R^{L} is aliphatic and aromatic alkyl, alkylester, alkylamine, dimethylamino alkyl, alkylether, alkylthioether, heteroaromatic alkyl, allyl, vinyl, alkyl groups functionalized with disulfide, oxime, ketone, acetal, hemiacetal, cleavable peptides, or cleavable sugars.

Embodiment 240: The oligonucleotide of any one of Embodiments 219-239, wherein one of R¹³ and R¹⁴ is an optionally substituted C₁-C₆alkyl.

Embodiment 241: The oligonucleotide of Embodiment 240, wherein one of R¹³ and R¹⁴ is methyl.

Embodiment 242: The oligonucleotide of any one of Embodiments 201-241, wherein X^{S} is O or CH₂.

Embodiment 243: The oligonucleotide of any one of Embodiments 201-242, wherein X^{S} is O.

Embodiment 244: The oligonucleotide of any one of Embodiments 201-243, wherein R²³ is a bond to an internucleotide linkage to a subsequent nucleoside.

Embodiment 245: The oligonucleotide of Embodiment 244, wherein R²² is hydroxyl, protected hydroxyl, halogen, optionally substituted C₁₋₃₀ alkoxy (e.g., methoxy, 2-methoxyethoxy), alkoxyalkyl (e.g., 2-methoxyethyl), hydrogen, optionally substituted C₁₋₃₀ alkyl, optionally substituted C₂₋₃₀alkenyl, optionally substituted C₂₋₃₀alkynyl, alkoxyalkylamine, alkoxyoxycarboxylate, amino, alkylamino, dialkylamino, 5-8 membered heterocyclyl, —O—C₄₋₃₀alkyl-ON(CH₂R⁸)(CH₂R⁹), or —O—C₄₋₃₀alkyl-ON(CH₂R⁸)(CH₂R⁹); or R²⁴ and R²⁵ taken together are 4′-C(R¹⁰R¹¹)ᵥ—Y-2′ or 4′-Y—C(R¹⁰R¹¹)ᵥ-2′.

Embodiment 246: The oligonucleotide of any one of Embodiments 244-245, wherein R²² is hydroxyl, protected hydroxyl, halogen, optionally substituted C₁₋₃₀ alkoxy (e.g., methoxy, 2-methoxyethoxy), alkoxyalkyl (e.g., 2-methoxyethyl), hydrogen, amino, alkylamino, or dialkylamino; or R²² and R²⁴ taken together are 4′-C(R¹⁰R¹¹)ᵥ—Y-2′ or 4′-Y—C(R¹⁰R¹¹)ᵥ-2′.

Embodiment 247: The oligonucleotide of any one of Embodiments 244-246, wherein R²² is hydrogen, hydroxyl, protected hydroxyl, fluoro, methoxy, ethoxy, or 2-methoxyethoxy; or R²² and R²⁴ taken together are 4′-C(R¹⁰R¹¹)ᵥ—Y-2′ or 4′-Y—C(R¹⁰R¹¹)ᵥ-2′.

Embodiment 248: The oligonucleotide of any one of Embodiments 244-247, wherein R²² is hydrogen, hydroxyl, protected hydroxyl, fluoro, or methoxy.

Embodiment 249: The oligonucleotide of any one of Embodiments 244-247, wherein R²² and R²⁴ taken together are 4′-C(R¹⁰R¹¹)ᵥ—Y-2′ or 4′-Y—C(R¹⁰R¹¹)ᵥ-2′.

Embodiment 250: The oligonucleotide of Embodiment 249, wherein R² and R⁴ taken together are 4′-C(R¹⁰R¹¹)ᵥ—Y-2′, wherein v is 1 or 2.

Embodiment 251: The oligonucleotide of Embodiment 249 or 250, wherein one of R¹⁰ and R¹¹ is H and the other is independently H or optionally substituted C₁-C₆alkyl.

Embodiment 252: The oligonucleotide of Embodiment 251, wherein R²² and R²⁴ taken together are 4′-CH₂—O-2′.

Embodiment 253: The oligonucleotide of any one of Embodiments 244-248, wherein R²⁴ is H.

Embodiment 254: The oligonucleotide of any one of Embodiments 201-243, wherein R²² is a bond to an internucleotide linkage to a subsequent nucleoside.

Embodiment 255: The oligonucleotide of Embodiment 254, wherein R²³ is hydroxyl, protected hydroxyl, halogen, optionally substituted C₁₋₃₀ alkoxy (e.g., methoxy, 2-methoxyethoxy), alkoxyalkyl (e.g., 2-methoxyethyl), hydrogen, optionally substituted C₁₋₃₀ alkyl, optionally substituted C₂₋₃₀alkenyl, optionally substituted C₂₋₃₀alkynyl, alkoxyalkylamine, alkoxyoxycarboxylate, amino, alkylamino, dialkylamino, 5-8 membered heterocyclyl, —O—C₄₋₃₀alkyl-ON(CH₂R⁸) (CH₂R⁹), or —O—C₄₋₃₀alkyl-ON(CH₂R⁸) (CH₂R⁹).

Embodiment 256: The oligonucleotide of any one of Embodiments 254-255, wherein R²³ is hydroxyl, protected hydroxyl, halogen, optionally substituted C₁₋₃₀ alkoxy (e.g., methoxy, 2-methoxyethoxy), alkoxyalkyl (e.g., 2-methoxyethyl), hydrogen, amino, alkylamino, or dialkylamino.

Embodiment 257: The oligonucleotide of any one of Embodiments 254-256, wherein R²³ is hydrogen, hydroxyl, protected hydroxyl, fluoro, methoxy, ethoxy, or 2-methoxyethoxy; or R² and R⁴ taken together are 4′-C(R¹⁰R¹¹)ᵥ—Y-2′ or 4′-Y—C(R¹⁰R¹¹)ᵥ-2′.

Embodiment 258: The oligonucleotide of any one of Embodiments 254-257, wherein R²³ is hydrogen, hydroxyl, protected hydroxyl, fluoro, or methoxy.

Embodiment 259: The oligonucleotide of any one of Embodiments 254-258, wherein R²⁴ is H.

Embodiment 260: The oligonucleotide of Embodiment 201, wherein the nucleoside of Formula (II) is selected from formulae (II-A)-(II-D):

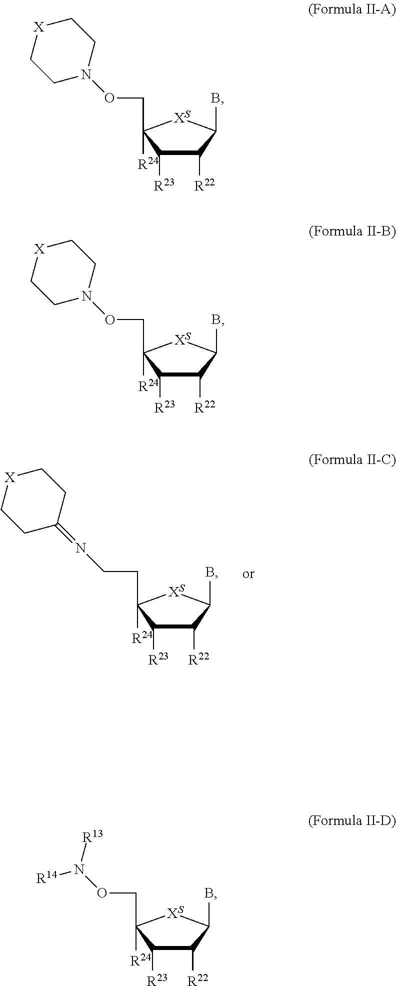

wherein:
R²² is hydrogen, hydroxyl, protected hydroxyl, halogen, optionally substituted C₁₋₃₀ alkoxy (e.g., methoxy, 2-methoxyethoxy), alkoxyalkyl (e.g., 2-methoxyethyl), amino, alkylamino, or dialkylamino;
R²³² is a bond to an internucleotide linkage to a subsequent nucleoside;
R²⁴ is hydrogen or R²² and R²⁴ taken together are 4′-C(R¹⁰R¹¹)ᵥ—Y-2′ or 4′-Y—C(R¹⁰R¹¹)ᵥ-2′.

Embodiment 261: The oligonucleotide of Embodiment 260, wherein X^{S} is O.

Embodiment 262: The oligonucleotide of Embodiment 260 or 261, wherein R²² is hydrogen, hydroxyl, protected hydroxyl, halogen, optionally substituted C₁₋₃₀ alkoxy (e.g., methoxy, 2-methoxyethoxy).

Embodiment 263: The oligonucleotide of any one of Embodiments 260-262, wherein R²² is hydrogen, hydroxyl, protected hydroxyl, fluoro, or methoxy.

Embodiment 264: The oligonucleotide of any one of Embodiments 260-263, wherein R²⁴ is H.

Embodiment 265: The oligonucleotide of Embodiments 260 or 261, wherein R²² and R²⁴ taken together are 4′-C(R¹⁰R¹¹)ᵥ—Y-2′ or 4′-Y—C(R¹⁰R¹¹)ᵥ-2′.

Embodiment 266: The oligonucleotide of Embodiment 265, wherein one of R¹⁰ and R¹¹ is H and the other is independently H or optionally substituted C₁-C₆alkyl.

Embodiment 267: The oligonucleotide of Embodiment 266, wherein R²² and R²⁴ taken together are 4′-CH₂—O-2′.

Embodiment 268: The oligonucleotide of any one of Embodiments 260-267, wherein one of R¹³ and R¹⁴ is

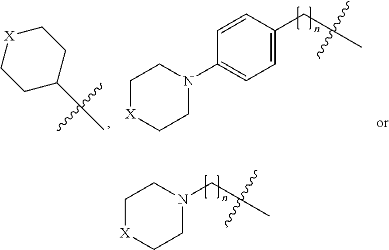

and the other of R¹³ and R¹⁴ is C₁-C₆alkyl,

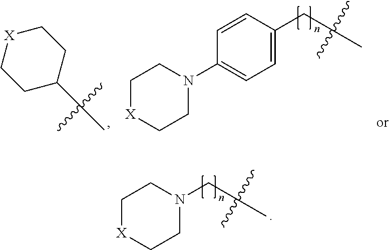

Embodiment 269: The oligonucleotide of any one of Embodiments 260-268, wherein R¹³ and R¹⁴ are the same.

Embodiment 270: The oligonucleotide of any one of Embodiments 260-268, wherein R¹³ and R¹⁴ are different.

Embodiment 271: The oligonucleotide of Embodiment 265, wherein one of R¹³ and R¹⁴ is C₁-C₆alkyl (e.g., methyl).

Embodiment 272: The oligonucleotide of any one of Embodiments 260-271, wherein X is NR^{L}.

Embodiment 273: The oligonucleotide of Embodiment 272, wherein R^{L} is a ligand or linker covalently bonded to one or more independently selected ligands.

Embodiment 274: The oligonucleotide of Embodiment 272, wherein R^{L} is aliphatic and aromatic alkyl, alkylester, alkylamine, dimethylamino alkyl, alkylether, alkylthioether, heteroaromatic alkyl, allyl, vinyl, alkyl groups functionalized with disulfide, oxime, ketone, acetal, hemiacetal, cleavable peptides, or cleavable sugars.

Embodiment 275: The oligonucleotide of any one of Embodiments 260-271, wherein X is O, S, CH₂ or NH.

Embodiment 276: The oligonucleotide of Embodiment 201, wherein the nucleoside is of Formula (II-E):

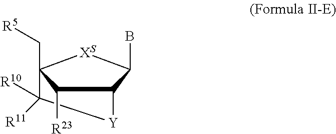

wherein:
R²³ is a bond to an internucleotide linkage to a subsequent nucleoside;
R⁵ is -L¹-R^{H}; and
X^{S}, B, Y, R¹⁰ and R¹¹ are as defined in claim 111.

Embodiment 277: The oligonucleotide of Embodiment 276, wherein the nucleoside is of Formula (II-Ea), (II-E′) or (II-E″:

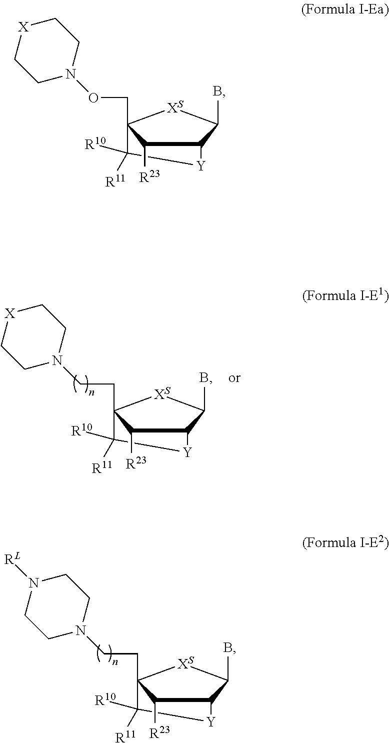

wherein
   n is 0 or an integer selected from 1 to 30 (e.g., n is 1, 2, 3, 4, 5, 6, 7, 8, 9, 10, 11, 12, 13, 14, 15, 16, 16, 17, 18, 19, 20, 21, 22, 23, 24, 25, 26, 27, 28, 29 or 30, such as n is 1, 2, 3, 4, 5 or 6, preferably n is 0 or 1);
X is O, NR^{L}, S, or CH₂; and
   R^{L} is H, a ligand, a linker covalently bonded to one or more ligands, aliphatic and aromatic alkyl, alkylester, alkylamine, dimethylamino alkyl, alkylether, alkylthioether, heteroaromatic alkyl, allyl, vinyl, alkyl groups functionalized with disulfide, oxime, ketone, acetal, hemiacetal, cleavable peptides, or cleavable sugars, and
optionally, the compound is of Formula (II-Eb) or (II-Ec):

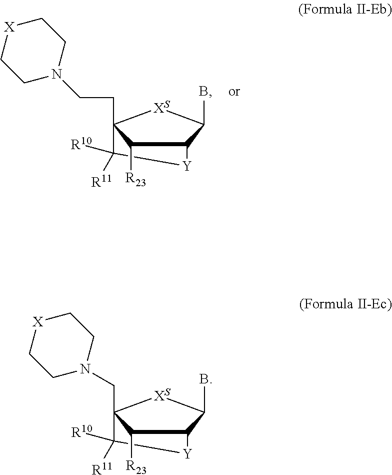

Embodiment 278: The oligonucleotide of Embodiment 277, wherein X is O or CH₂.

Embodiment 279: The oligonucleotide of Embodiment 277 or 278, wherein X is O.

Embodiment 280: The oligonucleotide of any one of Embodiments 276-279, wherein Y is O.

Embodiment 281: The oligonucleotide of any one of Embodiments 276-280, wherein one of R¹⁰ is H and the other is H or C₁₋₆alkyl (e.g., methyl).

Embodiment 282: The oligonucleotide of any one of Embodiments 276-281, wherein X^{S} is O.

Embodiment 283: The oligonucleotide of Embodiment 276, wherein the nucleoside is of Formula (II-Ed), (II-Ee), (II-E³) or (II-E⁴):

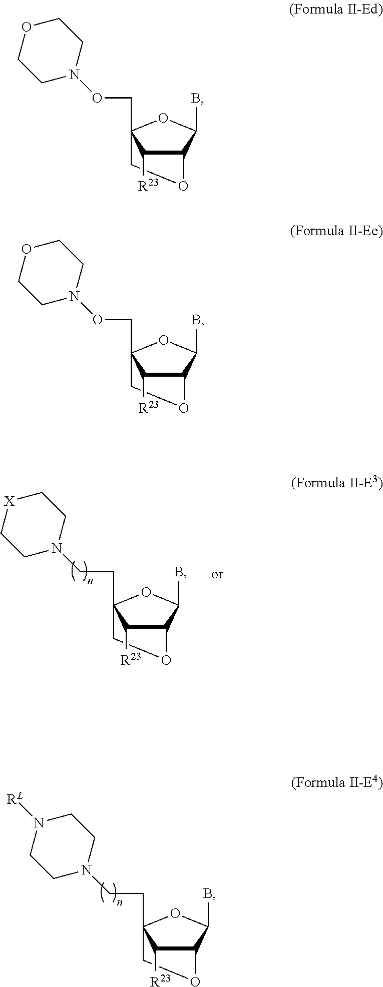

where n is 0 or an integer selected from 1 to 30 (e.g., n is 1, 2, 3, 4, 5, 6, 7, 8, 9, 10, 11, 12, 13, 14, 15, 16, 16, 17, 18, 19, 20, 21, 22, 23, 24, 25, 26, 27, 28, 29 or 30, such as n is 1, 2, 3, 4, 5 or 6, preferably n is 0 or 1); and
R^{L} is H, a ligand, a linker covalently bonded to one or more ligands, aliphatic and aromatic alkyl, alkylester, alkylamine, dimethylamino alkyl, alkylether, alkylthioether, heteroaromatic alkyl, allyl, vinyl, alkyl groups functionalized with disulfide, oxime, ketone, acetal, hemiacetal, cleavable peptides, or cleavable sugars, and optionally, the compound is of Formula (II-Ef) or (II-Eg):

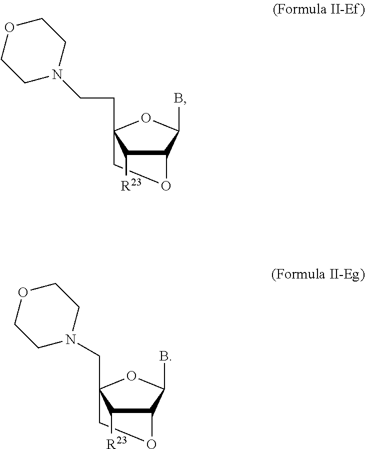

Embodiment 284: The oligonucleotide of any one of Embodiments 201-283, wherein the oligonucleotide comprises from 3 to 50 nucleotides.

Embodiment 285: The oligonucleotide of any one of Embodiments 201-284, wherein the oligonucleotide comprises at least one ribonucleotide.

Embodiment 286: The oligonucleotide of any one of Embodiments 201-285, wherein the oligonucleotide comprises at least one 2′-deoxyribonucleotide.

Embodiment 287: The oligonucleotide of any one of Embodiments 201-286, wherein the oligonucleotide comprises at least one nucleoside with a modified or non-natural nucleobase in addition to the nucleoside of Formula (II).

Embodiment 288: The oligonucleotide of any one of Embodiments 201-287, wherein the oligonucleotide comprises at least one nucleoside with a modified ribose sugar in addition to the nucleoside of Formula (II).

Embodiment 289: The oligonucleotide of any one of Embodiments 201-288, wherein the oligonucleotide comprises at least one nucleoside comprising a group other than H or OH at the 2′-position of the ribose sugar in addition to the nucleoside of Formula (II).

Embodiment 290: The oligonucleotide of any one of Embodiments 201-289, wherein the oligonucleotide comprises at least one nucleoside with a 2′-F ribose in addition to the nucleoside of Formula (II).

Embodiment 291: The oligonucleotide of any one of Embodiments 201-290, wherein the oligonucleotide comprises at least one nucleoside with a 2′-OMe ribose in addition to the nucleoside of Formula (II).

Embodiment 292: The oligonucleotide of any one of Embodiments 201-291, wherein the oligonucleotide comprises at least one nucleoside comprising a moiety other than a ribose sugar in addition to the nucleoside of Formula (II).

Embodiment 293: The oligonucleotide of any one of Embodiments 201-292, wherein the oligonucleotide comprises at least one modified internucleotide linkage.

Embodiment 294: The oligonucleotide of any one of Embodiments 201-293, wherein the internucleotide linkage to the subsequent nucleoside is a modified internucleotide linkage.

Embodiment 295: The oligonucleotide of Embodiment 294, wherein the modified internucleotide linkage is a phosphorothioate linkage.

Embodiment 296: The oligonucleotide of any one of Embodiments 201-295, wherein the oligonucleotide is attached to a solid support.

Embodiment 297: The oligonucleotide of any one of Embodiments 201-296, wherein oligonucleotide comprises at least one ligand.

Embodiment 298: The oligonucleotide of any one of Embodiments 201-297, wherein the oligonucleotide comprises at least one hydroxyl, phosphate or amino protecting group.

Embodiment 299: A double-stranded nucleic acid comprising a first oligonucleotide strand and a second oligonucleotide strand substantially complementary to the first strand, wherein the first or second strand is an oligonucleotide of any one of Embodiments 201-298.

Embodiment 300: The double-stranded nucleic acid of Embodiment 298, wherein one of the first stand and second strand is the oligonucleotide of any one of Embodiments 201-298 and the other strand comprises on its 5′-end a vinylphosphonate group (VP) group (e.g., *═CH—X^{P}, X^{P} is a phosphate group and * is C5′), C₃₋₆ cycloalkylphosphonate (e.g., cyclopropylphosphonate), monophosphate (HO)₂(O) P—O-5′), diphosphate (HO)₂(O) P—O—P(HO)(O)—O-5′), triphosphate ((HO)₂(O) P—O—(HO)(O) P—O—P(HO)(O)—O-5′); monothiophosphate (phosphorothioate, (HO)₂(S) P—O-5′), monodithiophosphate (phosphorodithioate; (HO)(HS)(S) P—O-5′), phosphorothiolate ((HO)₂(O) P—S-5′); alpha-thiotriphosphate; beta-thiotriphosphate; gamma-thiotriphosphate; phosphoramidates ((HO)₂(O) P—NH-5′, (HO)(NH₂)(O) P—O-5′), alkylphosphonates [(R^{P})(OH)(O) P—O-5′, R^{P} is optionally substituted C₁₋₃₀ alkyl, e.g., methyl, ethyl, isopropyl, or propyl)], alkyletherphosphonates [(R^{P1})(OH)(O) P—O-5′, R^{P1} is alkoxyalkyl, e.g., methoxymethyl (CH₂OMe) or ethoxymethyl], (HO)₂(X) P—O[—(CH₂)ₐ—O—P(X)(OH)—O]_{b}-5′ or (HO)₂(X) P—O[—(CH₂)ₐ—P(X)(OH)—O]_{b}-5′ or (HO)₂(X) P—[—(CH₂)ₐ—O—P(X)(OH)—O]_{b}-5′, or optionally substituted alkyl, and dialkyl terminal phosphates and phosphate mimics (e.g., HO[—(CH₂)ₐ—O—P(X)(OH)—O]_{b}-5′, H₂N[—(CH₂)ₐ—O—P(X)(OH)—O]_{b}-5′, H[—(CH₂)ₐ—O—P(X)(OH)—O]_{b}-5′, Me₂N[—(CH₂)ₐ—O—P(X)(OH)—O]_{b}-5′, HO[—(CH₂)ₐ—P(X)(OH)—O]_{b}-5′, H₂N[—(CH₂)ₐ—P(X)(OH)—O]_{b}-5′, H[—(CH₂)ₐ—P(X)(OH)—O]_{b}-5′, Me₂N[—(CH₂)ₐ—P(X)(OH)—O]_{b}-5′, wherein X is O or S; and a and b are each independently 1-10, optionally, the strand comprised a vinylphosphonate group, e.g., an E-vinylphosphonate group.

Embodiment 301: The double-stranded nucleic acid of Embodiment 299 or 300, wherein the first and second strand are independently 15 to 25 nucleotides in length.

Embodiment 302: The double-stranded nucleic acid any one of Embodiments 299-301, wherein double-stranded nucleic acid is capable of inducing RNA interference.

Embodiment 303: The double-stranded nucleic acid of Embodiment 302, wherein the double-stranded nucleic acid comprises an antisense strand and sense strand, and wherein the sense strand is the oligonucleotide of any one of Embodiments 201-298.

Embodiment 304: The double-stranded nucleic acid of Embodiment 303, wherein the antisense strand comprises at its 5′-end a vinylphosphonate group (VP) group (e.g., *═CH—X^{P}, X^{P} is a phosphate group and * is C5′), C₃₋₆ cycloalkylphosphonate (e.g., cyclopropylphosphonate), monophosphate ((HO)₂(O) P—O-5′), diphosphate ((HO)₂(O) P—O—P(HO)(O)—O-5′), triphosphate ((HO)₂(O) P—O—(HO)(O) P—O—P(HO)(O)—O-5′); monothiophosphate (phosphorothioate, (HO)2(S) P—O-5′), monodithiophosphate (phosphorodithioate; (HO)(HS)(S) P—O-5′), phosphorothiolate ((HO)₂(O) P—S-5′); alpha-thiotriphosphate; beta-thiotriphosphate; gamma-thiotriphosphate; phosphoramidates ((HO)₂(O) P—NH-5′, (HO)(NH₂)(O) P—O-5′), alkylphosphonates [(R^{P})(OH)(O) P—O-5′, R^{P} is optionally substituted C₁₋₃₀ alkyl, e.g., methyl, ethyl, isopropyl, or propyl)], alkyletherphosphonates [(R^{P1})(OH)(O) P—O-5′, R^{P1} is alkoxyalkyl, e.g., methoxymethyl (CH₂OMe) or ethoxymethyl], (HO)₂(X) P—O[—(CH₂)ₐ—O—P(X)(OH)—O]_{b}-5′ or (HO)₂(X) P—O[—(CH₂)ₐ—P(X)(OH)—O]_{b}-5′ or (HO)₂(X) P—[—(CH₂)ₐ—O—P(X)(OH)—O]_{b}-5′, or optionally substituted alkyl, and dialkyl terminal phosphates and phosphate mimics (e.g., HO[—(CH₂)ₐ—O—P(X)(OH)—O]_{b}-5′, H₂N[—(CH₂)ₐ—O—P(X)(OH)—O]_{b}-5′, H[—(CH₂)ₐ—O—P(X)(OH)—O]_{b}-5′, Me₂N[—(CH₂)ₐ—O—P(X)(OH)—O]_{b}-5′, HO[—(CH₂)ₐ-P(X)(OH)—O]_{b}-5′, H₂N[—(CH₂)ₐ—P(X)(OH)—O]_{α}-5′, H[—(CH₂)ₐ—P(X)(OH)—O]_{b}-5′, Me₂N[—(CH₂)ₐ—P(X)(OH)—O]_{b}-5′, wherein X is O or S; and a and b are each independently 1-10, optionally, the antisense strand comprises at its 5′-end a vinylphosphonate group, e.g., a Z-vinylphosphonate group.

Embodiment 305: The double-stranded nucleic acid of any one of Embodiments 299-304, wherein one or both strands have a 1-5 nucleotide overhang on its respective 5′-end or 3′-end.

Embodiment 306: The double-stranded nucleic acid of any one of Embodiments 299-305, wherein only one strand has a 2 nucleotide overhang on its 5′-end or 3′-end.

Embodiment 307: The double-stranded nucleic acid of any one of Embodiments 299-306, wherein only one strand has a 2 nucleotide overhand on its 3′-end.

Embodiment 308: A pharmaceutical composition comprising an oligonucleotide of any one of Embodiments 201-298 or dsRNA molecule of any one of Embodiments 1-110 or 299-307, alone or in combination with a pharmaceutically acceptable carrier or excipient.

Embodiment 309: A gene silencing kit containing an oligonucleotide of any one of Embodiments 201-298 or dsRNA molecule of any one of Embodiments 1-110 or 299-307.

Embodiment 310: A method for silencing a target gene in a cell, the method comprising a step of introducing into the cell:
(i) a double-stranded RNA according to any one of Embodiments 1-110 or 299-307, wherein the antisense strand comprises a nucleotide sequence substantially complementary to the target gene; or
(ii) an oligonucleotide according to any one of Embodiments 201-298, wherein the oligonucleotide comprises a nucleotide sequence substantially complementary to the target gene.

Embodiment 311: A method of reducing the expression of a target gene in a subject, comprising administering to the subject either:
a double-stranded RNA according to any one of Embodiments 1-110 or 299-307, wherein the antisense strand comprises a nucleotide sequence substantially complementary to the target gene; or
an oligonucleotide according to any one of Embodiments 201-298, wherein the oligonucleotide comprises a nucleotide sequence substantially complementary to a target gene.

Embodiment 312: The method of Embodiment 311, wherein said administering is subcutaneous or intravenous administration.

### Some Selected Definitions

For convenience, certain terms employed herein, in the specification, examples and appended claims are collected herein. Unless stated otherwise, or implicit from context, the following terms and phrases include the meanings provided below. Unless explicitly stated otherwise, or apparent from context, the terms and phrases below do not exclude the meaning that the term or phrase has acquired in the art to which it pertains. The definitions are provided to aid in describing particular embodiments, and are not intended to limit the claimed invention, because the scope of the invention is limited only by the claims. Further, unless otherwise required by context, singular terms shall include pluralities and plural terms shall include the singular.

Unless defined otherwise, all technical and scientific terms used herein have the same meaning as those commonly understood to one of ordinary skill in the art to which this invention pertains. Although any known methods, devices, and materials may be used in the practice or testing of the invention, the methods, devices, and materials in this regard are described herein. Definitions of common terms in immunology and molecular biology can be found in The Merck Manual of Diagnosis and Therapy, 20th Edition, published by Merck Sharp & Dohme Corp., 2018 (ISBN 0911910190, 978-0911910421); Robert S. Porter et al. (eds.), The Encyclopedia of Molecular Cell Biology and Molecular Medicine, published by Blackwell Science Ltd., 1999-2012 (ISBN 9783527600908); and Robert A. Meyers (ed.), Molecular Biology and Biotechnology: a Comprehensive Desk Reference, published by VCH Publishers, Inc., 1995 (ISBN 1-56081-569-8); Immunology by Werner Luttmann, published by Elsevier, 2006; Janeway's Immunobiology, Kenneth Murphy, Allan Mowat, Casey Weaver (eds.), W. W. Norton & Company, 2016 (ISBN 0815345054, 978-0815345053); Lewin's Genes XI, published by Jones & Bartlett Publishers, 2014 (ISBN-1449659055); Michael Richard Green and Joseph Sambrook, Molecular Cloning: A Laboratory Manual, 4th ed., Cold Spring Harbor Laboratory Press, Cold Spring Harbor, N.Y., USA (2012) (ISBN 1936113414); Davis et al., Basic Methods in Molecular Biology, Elsevier Science Publishing, Inc., New York, USA (2012) (ISBN 044460149X); Laboratory Methods in Enzymology: DNA, Jon Lorsch (ed.) Elsevier, 2013 (ISBN 0124199542); Current Protocols in Molecular Biology (CPMB), Frederick M. Ausubel (ed.), John Wiley and Sons, 2014 (ISBN 047150338X, 9780471503385), Current Protocols in Protein Science (CPPS), John E. Coligan (ed.), John Wiley and Sons, Inc., 2005; and Current Protocols in Immunology (CPI) (John E. Coligan, ADA M Kruisbeek, David H Margulies, Ethan M Shevach, Warren Strobe, (eds.) John Wiley and Sons, Inc., 2003 (ISBN 0471142735, 9780471142737), the contents of which are all incorporated by reference herein in their entireties.

Further, the practice of the present invention can employ, unless otherwise indicated, conventional techniques of molecular biology (including recombinant techniques), microbiology, cell biology, biochemistry, and immunology, which are within the skill of the art. Such techniques are explained fully in the literature, such as, “Molecular Cloning: A Laboratory Manual”, second edition (Sambrook et al., 1989); “Oligonucleotide Synthesis” (M. J. Gait, ed., 1984); “Animal Cell Culture” (R. I. Freshney, ed., 1987); “Methods in Enzymology” (Academic Press, Inc.); “Current Protocols in Molecular Biology” (F. M. Ausubel et al., eds., 1987, and periodic updates); “PCR: The Polymerase Chain Reaction”, (Mullis et al., ed., 1994); “A Practical Guide to Molecular Cloning” (Perbal Bernard V., 1988); “Phage Display: A Laboratory Manual” (Barbas et al., 2001).

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges and are also encompassed within the invention, subject to any specifically excluded limit in the stated range. Where the stated range includes one or both of the limits, ranges excluding either or both of those included limits are also included in the invention.

Other than in the operating examples, or where otherwise indicated, all numbers expressing quantities of ingredients or reaction conditions used herein should be understood as modified in all instances by the term “about.” The term “about” when used in connection with percentages can mean±1%. In some embodiments of the various aspects described herein, the term “about” when used in connection with percentages can mean±5%. The term “about” is used herein to provide literal support for the exact number that it precedes, as well as a number that is near to or approximately the number that the term precedes. In determining whether a number is near to or approximately a specifically recited number, the near or approximating unrecited number may be a number which, in the context in which it is presented, provides the substantial equivalent of the specifically recited number.

As used herein the term “comprising” or “comprises” is used in reference to compositions, methods, and respective component(s) thereof, that are essential to the invention, yet open to the inclusion of unspecified elements, whether essential or not.

The term “consisting of” refers to compositions, methods, and respective components thereof as described herein, which are exclusive of any element not recited in that description of the embodiment.

As used herein the term “consisting essentially of” refers to those elements required for a given embodiment. The term permits the presence of additional elements that do not materially affect the basic and novel or functional characteristic(s) of that embodiment of the invention.

The singular terms “a,” “an,” and “the” include plural referents unless context clearly indicates otherwise. Similarly, the word “or” is intended to include “and” unless the context clearly indicates otherwise. It is further noted that the claims can be drafted to exclude any optional element. As such, this statement is intended to serve as antecedent basis for use of such exclusive terminology as “solely,” “only” and the like in connection with the recitation of claim elements, or use of a “negative” limitation.

The abbreviation, “e.g.” is derived from the Latin exempli gratia, and is used herein to indicate a non-limiting example. Thus, the abbreviation “e.g.” is synonymous with the term “for example.”

As used herein, the terms “siRNA”, and “iRNA agent” are used interchangeably to refer to agents that can mediate silencing of a target RNA, e.g., mRNA, e.g., a transcript of a gene that encodes a protein. For convenience, such mRNA is also referred to herein as mRNA to be silenced. Such a gene is also referred to as a target gene. In general, the RNA to be silenced is an endogenous gene, exogenous gene or a pathogen gene. In addition, RNAs other than mRNA, e.g., tRNAs, and viral RNAs, can also be targeted.

As used herein, the phrase “mediates RNAi” refers to the ability to silence, in a sequence specific manner, a target gene, e.g., mRNA. While not wishing to be bound by theory, it is believed that silencing uses the RNAi machinery or process and a guide RNA, e.g., antisense strand of a dsRNA, where the antisense strand is 21 to 23 nucleotides in length.

By “specifically hybridizable” and “complementary” is meant that a nucleic acid can form hydrogen bond(s) with another nucleic acid sequence by either traditional Watson-Crick or other non-traditional types. In reference to the nucleic molecules of the present invention, the binding free energy for a nucleic acid molecule with its complementary sequence is sufficient to allow the relevant function of the nucleic acid to proceed, e.g., RNAi activity. Determination of binding free energies for nucleic acid molecules is well known in the art (see, e.g., Turner et al, 1987, CSH Symp. Quant. Biol. LII pp. 123-133; Frier et al., 1986, Proc. Nat. Acad. Sci. USA 83:9373-9377; Turner et al., 1987,/. Am. Chem. Soc. 109:3783-3785). A percent complementarity indicates the percentage of contiguous residues in a nucleic acid molecule that can form hydrogen bonds (e.g., Watson-Crick base pairing) with a second nucleic acid sequence (e.g., 5, 6, 7, 8, 9,10 out of 10 being 50%, 60%, 70%, 80%, 90%, and 100% complementary). “Perfectly complementary” or 100% complementarity means that all the contiguous residues of a nucleic acid sequence will hydrogen bond with the same number of contiguous residues in a second nucleic acid sequence. Less than perfect complementarity refers to the situation in which some, but not all, nucleoside units of two strands can hydrogen bond with each other. “Substantial complementarity” refers to polynucleotide strands exhibiting 90% or greater complementarity, excluding regions of the polynucleotide strands, such as overhangs, that are selected so as to be noncomplementary. Specific binding requires a sufficient degree of complementarity to avoid non-specific binding of the oligomeric compound to non-target sequences under conditions in which specific binding is desired, i.e., under physiological conditions in the case of in vivo assays or therapeutic treatment, or in the case of in vitro assays, under conditions in which the assays are performed. The non-target sequences typically differ by at least 5 nucleotides.

The term “off-target” and the phrase “off-target effects” refer to any instance in which an effector molecule against a given target causes an unintended affect by interacting either directly or indirectly with another target sequence, a DNA sequence or a cellular protein or other moiety. For example, an “off-target effect” may occur when there is a simultaneous degradation of other transcripts due to partial homology or complementarity between that other transcript and the sense and/or antisense strand of an siRNA.

The terms “decrease”, “reduced”, “reduction”, or “inhibit” are all used herein to mean a decrease by a statistically significant amount. In some embodiments, “reduce,” “reduction” or “decrease” or “inhibit” typically means a decrease by at least 10% as compared to a reference level (e.g. the absence of a given treatment or agent) and can include, for example, a decrease by at least about 10%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 98%, at least about 99%, or more. As used herein, “reduction” or “inhibition” does not encompass a complete inhibition or reduction as compared to a reference level. “Complete inhibition” is a 100% inhibition as compared to a reference level. A decrease can be preferably down to a level accepted as within the range of normal for an individual without a given disorder.

The terms “increased”, “increase”, “enhance”, or “activate” are all used herein to mean an increase by a statically significant amount. In some embodiments, the terms “increased”, “increase”, “enhance”, or “activate” can mean an increase of at least 10% as compared to a reference level, for example an increase of at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, or at least about a 2-fold, or at least about a 3-fold, or at least about a 4-fold, or at least about a 5-fold or at least about a 10-fold increase, or any increase between 2-fold and 10-fold or greater as compared to a reference level. In the context of a marker or symptom, a “increase” is a statistically significant increase in such level.

As used herein, a “terminal region” of a strand refers to positions 1-4, e.g., positions 1, 2, 3, and 4, counting from the nearest end of the strand. For example, a 5′-terminal region refers to positions 1-4, e.g., positions 1, 2, 3 and 4 counting from the 5′-end of the strand. Similarly, a 3′-terminal region refers to positions 1-4, e.g., positions 1, 2, 3 and 4 counting from the 3′-end of the strand.

For example, a 5′-terminal region for the antisense strand is positions 1, 2, 3 and 4 counting from the 5′-end of the antisense strand. A preferred 5′-terminal region for the antisense strand is positions 1, 2 and 3 counting from the 5′-end of the antisense strand. A 3′-terminal region for the antisense strand can be positions 1, 2, 3, and 4 counting from the 3′-end of the strand. A preferred 3′-terminal region for the antisense strand is positions 1, 2 and 3 counting from the 3′-end of the antisense strand.

Similarly, a 5′-terminal region for the sense strand is positions 1, 2, 3 and 4 counting from the 5′-end of the sense strand. A preferred 5′-terminal region for the sense strand is positions 1, 2 and 3 counting from the 5′-end of the sense strand. A 3′-terminal region for the sense strand can be positions 1, 2, 3, and 4 counting from the 3′-end of the strand. A preferred 3′-terminal region for the sense strand is positions 1, 2 and 3 counting from the 3′-end of the sense strand.

As used herein, a “central region” of a strand refers to positions 5-17, e.g., positions 6-16, positions 6-15, positions 6-14, positions 6-13, positions 6-12, positions 7-15, positions 7-14, positions 7-13, positions, 7-12, positions 8-16, positions 8-15, positions 8-14, positions 8-13, positions 8-12, positions 9-16, positions 9-15, positions 9-14, positions 9-13, positions 9-12, positions 10-16, positions 10-15, positions 10-14, positions 10-13 or positions 10-12, counting from the 5′-end of the strand. For example, the central region of a strand means positions 5, 6, 7, 8, 9, 10, 11, 12, 13, 14, 15, 16 or 17 of the strand. A preferred central region for the sense strand is positions 6, 7, 8, 9, 10, 11, 12, 13, and 14, counting from the 5′-end of the sense strand. A more preferred central region for the sense strand is positions 7, 8, 9, 10, 11, 12 and 13, counting from the 5′-end of the sense strand. A preferred central region for the antisense strand is positions 9, 10, 11, 12, 13, 14, 15 16 and 17, counting from 5′-end of the antisense strand. A more preferred central region for the antisense strand is positions 10, 11, 12, 13, 14, 15 and 16, counting from 5′-end of the antisense strand.

As used herein, the term “in vitro” refers to events that occur in an artificial environment, e.g., in a test tube or reaction vessel, in cell culture, etc., rather than within an organism (e.g. animal or a plant). As used herein, the term “ex vivo” refers to cells which are removed from a living organism and cultured outside the organism (e.g., in a test tube). As used herein, the term “in vivo” refers to events that occur within an organism (e.g. animal, plant, and/or microbe).

As used herein, the term “subject” or “patient” refers to any organism to which a composition disclosed herein can be administered, e.g., for experimental, diagnostic, and/or therapeutic purposes. Typical subjects include animals (e.g., mammals such as mice, rats, rabbits, non-human primates, and humans) and/or plants. Usually the animal is a vertebrate such as a primate, rodent, domestic animal or game animal. Primates include chimpanzees, cynomologous monkeys, spider monkeys, and macaques, e.g., Rhesus. Rodents include mice, rats, woodchucks, ferrets, rabbits and hamsters. Domestic and game animals include cows, horses, pigs, deer, bison, buffalo, feline species, e.g., domestic cat, canine species, e.g., dog, fox, wolf, avian species, e.g., chicken, emu, ostrich, and fish, e.g., trout, catfish and salmon. Patient or subject includes any subset of the foregoing, e.g., all of the above, but excluding one or more groups or species such as humans, primates or rodents. In certain embodiments of the aspects described herein, the subject is a mammal, e.g., a primate, e.g., a human. The terms, “patient” and “subject” are used interchangeably herein. A subject can be male or female.

Preferably, the subject is a mammal. The mammal can be a human, non-human primate, mouse, rat, dog, cat, horse, or cow, but are not limited to these examples. Mammals other than humans can be advantageously used as subjects that represent animal models of human diseases and disorders. In addition, compounds, compositions and methods described herein can be used to with domesticated animals and/or pets.

A subject can be one who has been previously diagnosed with or identified as suffering from or having a condition in need of treatment. Alternatively, a subject can also be one who has not been previously diagnosed. A “subject in need” of testing for a particular condition can be a subject having that condition, diagnosed as having that condition, or at risk of developing that condition.

In some embodiments, the subject is human. In another embodiment, the subject is an experimental animal or animal substitute as a disease model. The term does not denote a particular age or sex. Thus, adult and newborn subjects, as well as fetuses, whether male or female, are intended to be covered. Examples of subjects include humans, dogs, cats, cows, goats, and mice. The term subject is further intended to include transgenic species. In some embodiments, the subject can be of European ancestry. In some embodiments, the subject can be of African American ancestry. In some embodiments, the subject can be of Asian ancestry.

In jurisdictions that forbid the patenting of methods that are practiced on the human body, the meaning of “administering” of a composition to a human subject shall be restricted to prescribing a controlled substance that a human subject will self-administer by any technique (e.g., orally, inhalation, topical application, injection, insertion, etc.). The broadest reasonable interpretation that is consistent with laws or regulations defining patentable subject matter is intended. In jurisdictions that do not forbid the patenting of methods that are practiced on the human body, the “administering” of compositions includes both methods practiced on the human body and also the foregoing activities.

As used herein, the term “parenteral administration,” refers to administration through injection or infusion. Parenteral administration includes, but is not limited to, subcutaneous administration, intravenous administration, or intramuscular administration.

As used herein, the term “subcutaneous administration” refers to administration just below the skin. “Intravenous administration” means administration into a vein.

As used herein, the term “dose” refers to a specified quantity of a pharmaceutical agent provided in a single administration. In certain embodiments, a dose may be administered in two or more boluses, tablets, or injections. For example, in certain embodiments, where subcutaneous administration is desired, the desired dose requires a volume not easily accommodated by a single injection. In such embodiments, two or more injections may be used to achieve the desired dose. In certain embodiments, a dose may be administered in two or more injections to minimize injection site reaction in an individual.

As used herein, the term “dosage unit” refers to a form in which a pharmaceutical agent is provided. In certain embodiments, a dosage unit is a vial comprising lyophilized antisense oligonucleotide. In certain embodiments, a dosage unit is a vial comprising reconstituted antisense oligonucleotide.

By the terms “treat,” “treating” or “treatment of” (and grammatical variations thereof) it is meant that the severity of the subject's condition is reduced, at least partially improved or stabilized and/or that some alleviation, mitigation, decrease or stabilization in at least one clinical symptom is achieved and/or there is a delay in the progression of the disease or disorder.

The terms “prevent,” “preventing” and “prevention” (and grammatical variations thereof) refer to prevention and/or delay of the onset of a disease, disorder and/or a clinical symptom(s) in a subject and/or a reduction in the severity of the onset of the disease, disorder and/or clinical symptom(s) relative to what would occur in the absence of the methods of the invention. The prevention can be complete, e.g., the total absence of the disease, disorder and/or clinical symptom(s). The prevention can also be partial, such that the occurrence of the disease, disorder and/or clinical symptom(s) in the subject and/or the severity of onset is less than what would occur in the absence of the present invention.

The term “statistically significant” or “significantly” refers to statistical significance and generally means a two standard deviation (2SD) or greater difference.

As used herein, the term “aliphatic” means a saturated or unsaturated and straight, branched, and/or cyclic hydrocarbon having the defined number of carbon atom. Examples include alkyl, alkenyl, alkynyl, cycloalkyl, cycloalkylalkyl, cycloalkylalkenyl, and cycloalkylalkynyl, having the defined number of carbon atoms.

As used herein, the term “alkyl” refers to an aliphatic hydrocarbon group which can be straight or branched having 1 to about 60 carbon atoms in the chain, and which preferably have about 6 to about 50 carbons in the chain. “Lower alkyl” refers to an alkyl group having 1 to about 8 carbon atoms. “Higher alkyl” refers to an alkyl group having about 10 to about 20 carbon atoms. The alkyl group can be optionally substituted with one or more alkyl group substituents which can be the same or different, where “alkyl group substituent” includes halo, amino, aryl, hydroxyl, alkoxy, aryloxy, alkyloxy, alkylthio, arylthio, aralkyloxy, aralkylthio, carboxy, alkoxycarbonyl, oxo and cycloalkyl. “Branched” refers to an alkyl group in which a lower alkyl group, such as methyl, ethyl or propyl, is attached to a linear alkyl chain. Exemplary alkyl groups include methyl, ethyl, propyl, i-propyl, n-butyl, t-butyl, n-pentyl, hexyl, heptyl, octyl, decyl, dodecyl, tridecyl, tetradecyl, pentadecyl and hexadecyl. Useful alkyl groups include branched or straight chain alkyl groups of 6 to 50 carbon, and also include the lower alkyl groups of 1 to about 4 carbons and the higher alkyl groups of about 12 to about 16 carbons.

A “heteroalkyl” group substitutes any one of the carbons of the alkyl group with a heteroatom having the appropriate number of hydrogen atoms attached (e.g., a CH₂ group to an NH group or an O group). The term “heteroalkyl” include optionally substituted alkyl, alkenyl and alkynyl radicals which have one or more skeletal chain atoms selected from an atom other than carbon, e.g., oxygen, nitrogen, sulfur, phosphorus, silicon, or combinations thereof. In certain embodiments, the heteroatom(s) is placed at any interior position of the heteroalkyl group. Examples include, but are not limited to, —CH₂—O—CH₃, —CH₂—CH₂—O—CH₃, —CH₂—NH—CH₃, —CH₂—CH₂—NH—CH₃, —CH₂—N(CH₃)—CH₃, —CH₂—CH₂—NH—CH₃, —CH₂—CH₂—N(CH₃)—CH₃, —CH₂—S—CH₂—CH₃, —CH₂—CH₂, —S(O)—CH₃, —CH₂—CH₂—S(O)₂—CH₃, —CH═CH—O—CH₃, —Si(CH₃)₃, —CH₂—CH═N—OCH₃, and —CH═CH—N(CH₃)—CH₃. In some embodiments, up to two heteroatoms are consecutive, such as, by way of example, —CH₂—NH—OCH₃ and —CH₂—O—Si(CH₃)₃

As used herein, the term “alkenyl” refers to an alkyl group containing at least one carbon-carbon double bond. The alkenyl group can be optionally substituted with one or more “alkyl group substituents.” Exemplary alkenyl groups include vinyl, allyl, n-pentenyl, decenyl, dodecenyl, tetradecadienyl, heptadec-8-en-1-yl and heptadec-8,11-dien-1-yl.

As used herein, the term “alkynyl” refers to an alkyl group containing a carbon-carbon triple bond. The alkynyl group can be optionally substituted with one or more “alkyl group substituents.” Exemplary alkynyl groups include ethynyl, propargyl, n-pentynyl, decynyl and dodecynyl. Useful alkynyl groups include the lower alkynyl groups.

As used herein, the term “cycloalkyl” refers to a non-aromatic mono- or multicyclic ring system of about 3 to about 12 carbon atoms. The cycloalkyl group can be optionally partially unsaturated. The cycloalkyl group can be also optionally substituted with an aryl group substituent, oxo and/or alkylene. Representative monocyclic cycloalkyl rings include cyclopentyl, cyclohexyl and cycloheptyl. Useful multicyclic cycloalkyl rings include adamantyl, octahydronaphthyl, decalin, camphor, camphane, and noradamantyl.

“Heterocyclyl” refers to a nonaromatic 3-8 membered monocyclic, 8-12 membered bicyclic, or 11-14 membered tricyclic ring system having 1-3 heteroatoms if monocyclic, 1-6 heteroatoms if bicyclic, or 1-9 heteroatoms if tricyclic, said heteroatoms selected from O, N, or S (e.g., carbon atoms and 1-3, 1-6, or 1-9 heteroatoms of N, O, or S if monocyclic, bicyclic, or tricyclic, respectively). Cₓheterocyclyl and Cₓ-C_{y}heterocyclyl are typically used where X and Y indicate the number of carbon atoms in the ring system. In some embodiments, 1, 2 or 3 hydrogen atoms of each ring can be substituted by a substituent. Exemplary heterocyclyl groups include, but are not limited to piperazinyl, pyrrolidinyl, dioxanyl, morpholinyl, tetrahydrofuranyl, piperidyl, 4-morpholyl, 4-piperazinyl, pyrrolidinyl, perhydropyrrolizinyl, 1,4-diazaperhydroepinyl, 1,3-dioxanyl, 1,4-dioxanyland the like.

“Aryl” refers to an aromatic carbocyclic radical containing about 3 to about 13 carbon atoms. The aryl group can be optionally substituted with one or more aryl group substituents, which can be the same or different, where “aryl group substituent” includes alkyl, alkenyl, alkynyl, aryl, aralkyl, hydroxyl, alkoxy, aryloxy, aralkoxy, carboxy, aroyl, halo, nitro, trihalomethyl, cyano, alkoxycarbonyl, aryloxycarbonyl, aralkoxycarbonyl, acyloxy, acylamino, aroylamino, carbamoyl, alkylcarbamoyl, dialkylcarbamoyl, rylthio, alkylthio, alkylene and —NRR′, where R and R′ are each independently hydrogen, alkyl, aryl and aralkyl. Exemplary aryl groups include substituted or unsubstituted phenyl and substituted or unsubstituted naphthyl.

“Heteroaryl” refers to an aromatic 3-8 membered monocyclic, 8-12 membered fused bicyclic, or 11-14 membered fused tricyclic ring system having 1-3 heteroatoms if monocyclic, 1-6 heteroatoms if bicyclic, or 1-9 heteroatoms if tricyclic, said heteroatoms selected from O, N, or S (e.g., carbon atoms and 1-3, 1-6, or 1-9 heteroatoms of N, O, or S if monocyclic, bicyclic, or tricyclic, respectively.

Exemplary aryl and heteroaryls include, but are not limited to, phenyl, pyridinyl, pyrimidinyl, furanyl, thienyl, imidazolyl, thiazolyl, pyrazolyl, pyridazinyl, pyrazinyl, triazinyl, tetrazolyl, indolyl, benzyl, naphthyl, anthracenyl, azulenyl, fluorenyl, indanyl, indenyl, naphthyl, tetrahydronaphthyl, benzimidazolyl, benzofuranyl, benzothiofuranyl, benzothiophenyl, benzoxazolyl, benzoxazolinyl, benzthiazolyl, benztriazolyl, benztetrazolyl, benzisoxazolyl, benzisothiazolyl, benzimidazolinyl, carbazolyl, 4aH carbazolyl, carbolinyl, chromanyl, chromenyl, cinnolinyl, decahydroquinolinyl, 2H,6H-1,5,2-dithiazinyl, dihydrofuro[2,3 b]tetrahydrofuran, furanyl, furazanyl, imidazolidinyl, imidazolinyl, imidazolyl, 1H-indazolyl, indolenyl, indolinyl, indolizinyl, indolyl, 3H-indolyl, isatinoyl, isobenzofuranyl, isochromanyl, isoindazolyl, isoindolinyl, isoindolyl, isoquinolinyl, isothiazolyl, isoxazolyl, methylenedioxyphenyl, morpholinyl, naphthyridinyl, octahydroisoquinolinyl, oxadiazolyl, 1,2,3-oxadiazolyl, 1,2,4-oxadiazolyl, 1,2,5-oxadiazolyl, 1,3,4-oxadiazolyl, oxazolidinyl, oxazolyl, oxindolyl, pyrimidinyl, phenanthridinyl, phenanthrolinyl, phenazinyl, phenothiazinyl, phenoxathinyl, phenoxazinyl, phthalazinyl, piperazinyl, piperidinyl, piperidonyl, 4-piperidonyl, piperonyl, pteridinyl, purinyl, pyranyl, pyrazinyl, pyrazolidinyl, pyrazolinyl, pyrazolyl, pyridazinyl, pyridooxazole, pyridoimidazole, pyridothiazole, pyridinyl, pyridyl, pyrimidinyl, pyrrolidinyl, pyrrolinyl, 2H-pyrrolyl, pyrrolyl, quinazolinyl, quinolinyl, 4H-quinolizinyl, quinoxalinyl, quinuclidinyl, tetrahydrofuranyl, tetrahydroisoquinolinyl, tetrahydroquinolinyl, tetrazolyl, 6H-1,2,5-thiadiazinyl, 1,2,3-thiadiazolyl, 1,2,4-thiadiazolyl, 1,2,5-thiadiazolyl, 1,3,4-thiadiazolyl, thianthrenyl, thiazolyl, thienyl, thienothiazolyl, thienooxazolyl, thienoimidazolyl, thiophenyl and xanthenyl, and the like. In some embodiments, 1, 2, 3, or 4 hydrogen atoms of each ring can be substituted by a substituent.

As used herein, the term “halogen” or “halo” refers to an atom selected from fluorine, chlorine, bromine and iodine. The term “halogen radioisotope” or “halo isotope” refers to a radionuclide of an atom selected from fluorine, chlorine, bromine and iodine.

A “halogen-substituted moiety” or “halo-substituted moiety”, as an isolated group or part of a larger group, means an aliphatic, alicyclic, or aromatic moiety, as described herein, substituted by one or more “halo” atoms, as such terms are defined in this application.

The term “haloalkyl” as used herein refers to alkyl and alkoxy structures structure with at least one substituent of fluorine, chorine, bromine or iodine, or with combinations thereof. In embodiments, where more than one halogen is included in the group, the halogens are the same or they are different. The terms “fluoroalkyl” and “fluoroalkoxy” include haloalkyl and haloalkoxy groups, respectively, in which the halo is fluorine. Exemplary halo-substituted alkyl includes haloalkyl, dihaloalkyl, trihaloalkyl, perhaloalkyl and the like (e.g. halosubstituted (C₁-C₃)alkyl includes chloromethyl, dichloromethyl, difluoromethyl, trifluoromethyl (CF₃), perfluoroethyl, 2,2,2-trifluoroethyl, 2,2,2-trifluoro-1,1-dichloroethyl, and the like).

As used herein, the term “amino” means —NH₂. The term “alkylamino” means a nitrogen moiety having one straight or branched unsaturated aliphatic, cyclyl, or heterocyclyl radicals attached to the nitrogen, e.g., —NH (alkyl). The term “dialkylamino” means a nitrogen moiety having at two straight or branched unsaturated aliphatic, cyclyl, or heterocyclyl radicals attached to the nitrogen, e.g., —N(alkyl) (alkyl). The term “alkylamino” includes “alkenylamino,” “alkynylamino,” “cyclylamino,” and “heterocyclylamino.” The term “arylamino” means a nitrogen moiety having at least one aryl radical attached to the nitrogen. For example, —NHaryl, and —N(aryl)₂. The term “heteroarylamino” means a nitrogen moiety having at least one heteroaryl radical attached to the nitrogen. For example —NHheteroaryl, and —N(heteroaryl)₂. Optionally, two substituents together with the nitrogen can also form a ring. Unless indicated otherwise, the compounds described herein containing amino moieties can include protected derivatives thereof. Suitable protecting groups for amino moieties include acetyl, tertbutoxycarbonyl, benzyloxycarbonyl, and the like. Exemplary alkylamino includes, but is not limited to, NH(C₁-C₁₀alkyl), such as —NHCH₃, —NHCH₂CH₃, —NHCH₂CH₂CH₃, and —NHCH(CH₃)₂.

Exemplary dialkylamino includes, but is not limited to, —N(C₁-C₁₀alkyl)₂, such as N(CH₃)₂, —N(CH₂CH₃)₂, —N(CH₂CH₂CH₃)₂, and —N(CH(CH₃)₂)₂.

The term “aminoalkyl” means an alkyl, alkenyl, and alkynyl as defined above, except where one or more substituted or unsubstituted nitrogen atoms (—N—) are positioned between carbon atoms of the alkyl, alkenyl, or alkynyl. For example, an (C₂-C₆)aminoalkyl refers to a chain comprising between 2 and 6 carbons and one or more nitrogen atoms positioned between the carbon atoms.

The terms “hydroxyl” and “hydroxyl” mean the radical —OH.

The terms “alkoxyl” or “alkoxy” as used herein refers to an alkyl group, as defined above, having an oxygen radical attached thereto, and can be represented by one of —O-alkyl, —O-alkenyl, and —O-alkynyl. Aroxy can be represented by —O-aryl or O-heteroaryl, wherein aryl and heteroaryl are as defined herein. The alkoxy and aroxy groups can be substituted as described above for alkyl. Exemplary alkoxy groups include, but are not limited to O-methyl, O-ethyl, O-n-propyl, O-isopropyl, O-n-butyl, O-isobutyl, O-sec-butyl, O-tert-butyl, O-pentyl, O-hexyl, O-cyclopropyl, O-cyclobutyl, O-cyclopentyl, O-cyclohexyl and the like.

As used herein, the term “carbonyl” means the radical —C(O)—. It is noted that the carbonyl radical can be further substituted with a variety of substituents to form different carbonyl groups including acids, acid halides, amides, esters, ketones, and the like.

As used herein, the term “oxo” means double bonded oxygen, i.e., ═O.

The term “carboxy” means the radical —C(O)O—. It is noted that compounds described herein containing carboxy moieties can include protected derivatives thereof, i.e., where the oxygen is substituted with a protecting group. Suitable protecting groups for carboxy moieties include benzyl, tert-butyl, and the like. As used herein, a carboxy group includes-COOH, i.e., carboxyl group.

The term “ester” refers to a chemical moiety with formula —C(═O)OR, where R is selected from the group consisting of alkyl, cycloalkyl, aryl, heteroaryl and heterocycloalkyl.

The term “cyano” means the radical —CN.

The term “nitro” means the radical —NO₂.

The term, “heteroatom” refers to an atom that is not a carbon atom. Particular examples of heteroatoms include, but are not limited to nitrogen, oxygen, sulfur and halogens. A “heteroatom moiety” includes a moiety where the atom by which the moiety is attached is not a carbon. Examples of heteroatom moieties include —N═, —NR^{N}—, —N⁺(O^{-)═, —O—, —S— or —S(O)}₂—, —OS(O)₂—, and —SS—, wherein RN is H or a further substituent.

The terms “alkylthio” and “thioalkoxy” refer to an alkoxy group, as defined above, where the oxygen atom is replaced with a sulfur. In preferred embodiments, the “alkylthio” moiety is represented by one of —S-alkyl, —S-alkenyl, and —S-alkynyl. Representative alkylthio groups include methylthio, ethylthio, and the like. The term “alkylthio” also encompasses cycloalkyl groups, alkene and cycloalkene groups, and alkyne groups. “Arylthio” refers to aryl or heteroaryl groups.

The term “sulfinyl” means the radical —SO—. It is noted that the sulfinyl radical can be further substituted with a variety of substituents to form different sulfinyl groups including sulfinic acids, sulfinamides, sulfinyl esters, sulfoxides, and the like.

The term “sulfonyl” means the radical —SO₂—. It is noted that the sulfonyl radical can be further substituted with a variety of substituents to form different sulfonyl groups including sulfonic acids (—SO₃H), sulfonamides, sulfonate esters, sulfones, and the like.

The term “thiocarbonyl” means the radical —C(S)—. It is noted that the thiocarbonyl radical can be further substituted with a variety of substituents to form different thiocarbonyl groups including thioacids, thioamides, thioesters, thioketones, and the like.

“Acyl” refers to an alkyl-CO— group, wherein alkyl is as previously described. Exemplary acyl groups comprise alkyl of 1 to about 30 carbon atoms. Exemplary acyl groups also include acetyl, propanoyl, 2-methylpropanoyl, butanoyl and palmitoyl.

“Aroyl” means an aryl-CO— group, wherein aryl is as previously described. Exemplary aroyl groups include benzoyl and 1- and 2-naphthoyl.

“Arylthio” refers to an aryl-S— group, wherein the aryl group is as previously described. Exemplary arylthio groups include phenylthio and naphthylthio.

“Aralkyl” refers to an aryl-alkyl- group, wherein aryl and alkyl are as previously described. Exemplary aralkyl groups include benzyl, phenylethyl and naphthylmethyl.

“Aralkyloxy” refers to an aralkyl-O— group, wherein the aralkyl group is as previously described. An exemplary aralkyloxy group is benzyloxy.

“Aralkylthio” refers to an aralkyl-S— group, wherein the aralkyl group is as previously described. An exemplary aralkylthio group is benzylthio.

“Alkoxycarbonyl” refers to an alkyl-O—CO— group. Exemplary alkoxycarbonyl groups include methoxycarbonyl, ethoxycarbonyl, butyloxycarbonyl, and t-butyloxycarbonyl.

“Aryloxycarbonyl” refers to an aryl-O—CO— group. Exemplary aryloxycarbonyl groups include phenoxy- and naphthoxy-carbonyl.

“Aralkoxycarbonyl” refers to an aralkyl-O—CO— group. An exemplary aralkoxycarbonyl group is benzyloxycarbonyl.

“Carbamoyl” refers to an H₂N—CO— group.

“Alkylcarbamoyl” refers to a R′RN—CO— group, wherein one of R and R′ is hydrogen and the other of R and R′ is alkyl as previously described.

“Dialkylcarbamoyl” refers to R′RN—CO— group, wherein each of R and R′ is independently alkyl as previously described.

“Acyloxy” refers to an acyl-O— group, wherein acyl is as previously described. “Acylamino” refers to an acyl-NH— group, wherein acyl is as previously described. “Aroylamino” refers to an aroyl-NH— group, wherein aroyl is as previously described.

The term “optionally substituted” means that the specified group or moiety is unsubstituted or is substituted with one or more (typically 1, 2, 3, 4, 5 or 6 substituents) independently selected from the group of substituents listed below in the definition for “substituents” or otherwise specified. The term “substituents” refers to a group “substituted” on a substituted group at any atom of the substituted group. Suitable substituents include, without limitation, halogen, hydroxyl, caboxy, oxo, nitro, haloalkyl, alkyl, alkenyl, alkynyl, alkaryl, aryl, heteroaryl, cyclyl, heterocyclyl, aralkyl, alkoxy, aryloxy, amino, acylamino, alkylcarbanoyl, arylcarbanoyl, aminoalkyl, alkoxycarbonyl, carboxy, hydroxylalkyl, alkanesulfonyl, arenesulfonyl, alkanesulfonamido, arenesulfonamido, aralkylsulfonamido, alkylcarbonyl, acyloxy, cyano or ureido. In some cases, two substituents, together with the carbons to which they are attached to can form a ring.

For example, any alkyl, alkenyl, cycloalkyl, heterocyclyl, heteroaryl or aryl is optionally substituted with 1, 2, 3, 4 or 5 groups selected from OH, CN, —SC(O)Ph, oxo (═O), SH, SO₂NH₂, SO₂(C₁-C₄)alkyl, SO₂NH(C₁-C₄)alkyl, halogen, carbonyl, thiol, cyano, NH₂, NH(C₁-C₄)alkyl, N[(C₁-C₄)alkyl]₂, C(O)NH₂, COOH, COOMe, acetyl, (C₁-C₅)alkyl, O(C₁-C₈)alkyl, O(C₁-C₈)haloalkyl, (C₂-C₈)alkenyl, (C₂-C₈)alkynyl, haloalkyl, thioalkyl, cyanomethylene, alkylaminyl, aryl, heteroaryl, substituted aryl, NH₂—C(O)-alkylene, NH(Me)-C(O)-alkylene, CH₂C(O)-alkyl, C(O)-alkyl, alkylcarbonylaminyl, CH₂—[CH(OH)]ₘ—(CH₂)ₚ—OH, CH₂—[CH(OH)]ₘ—(CH₂)ₚ—NH₂ or CH₂-aryl-alkoxy; “m” and “p” are independently 1, 2, 3, 4, 5 or 6.

In some embodiments, an optionally substituted group is substituted with 1 substituent. In some other embodiments, an optionally substituted group is substituted with 2 independently selected substituents, which can be same or different. In some other embodiments, an optionally substituted group is substituted with 3 independently selected substituents, which can be same, different or any combination of same and different. In still some other embodiments, an optionally substituted group is substituted with 4 independently selected substituents, which can be same, different or any combination of same and different. In yet some other embodiments, an optionally substituted group is substituted with 5 independently selected substituents, which can be same, different or any combination of same and different.

An “isocyanato” group refers to a NCO group.

A “thiocyanato” group refers to a CNS group.

An “isothiocyanato” group refers to a NCS group.

“Alkoyloxy” refers to a RC(═O)O— group.

“Alkoyl” refers to a RC(═O)— group.

It should be understood that this disclosure is not limited to the particular methodology, protocols, and reagents, etc., provided herein and as such may vary. The terminology used herein is for the purpose of describing particular embodiments only, and is not intended to limit the scope of the present disclosure, which is defined solely by the claims. The invention is further illustrated by the following example, which should not be construed as further limiting.

Groupings of alternative elements or embodiments of the invention disclosed herein are not to be construed as limitations. Each group member can be referred to and claimed individually or in any combination with other members of the group or other elements found herein. One or more members of a group can be included in, or deleted from, a group for reasons of convenience and/or patentability. When any such inclusion or deletion occurs, the specification is herein deemed to contain the group as modified thus fulfilling the written description of all Markush groups used in the appended claims.

The description of embodiments of the disclosure is not intended to be exhaustive or to limit the disclosure to the precise form disclosed. While specific embodiments of, and examples for, the disclosure are described herein for illustrative purposes, various equivalent modifications are possible within the scope of the disclosure, as those skilled in the relevant art will recognize. For example, while method steps or functions are presented in a given order, alternative embodiments may perform functions in a different order, or functions may be performed substantially concurrently. The teachings of the disclosure provided herein can be applied to other procedures or methods as appropriate. The various embodiments described herein can be combined to provide further embodiments. Aspects of the disclosure can be modified, if necessary, to employ the compositions, functions and concepts of the above references and application to provide yet further embodiments of the disclosure. Moreover, due to biological functional equivalency considerations, some changes can be made in protein structure without affecting the biological or chemical action in kind or amount. These and other changes can be made to the disclosure in light of the detailed description. All such modifications are intended to be included within the scope of the appended claims.

Specific elements of any of the foregoing embodiments can be combined or substituted for elements in other embodiments. Furthermore, while advantages associated with certain embodiments of the disclosure have been described in the context of these embodiments, other embodiments may also exhibit such advantages, and not all embodiments need necessarily exhibit such advantages to fall within the scope of the disclosure.

### EXAMPLES

The technology described herein is further illustrated by the following examples which in no way should be construed as being further limiting.

### Example 1

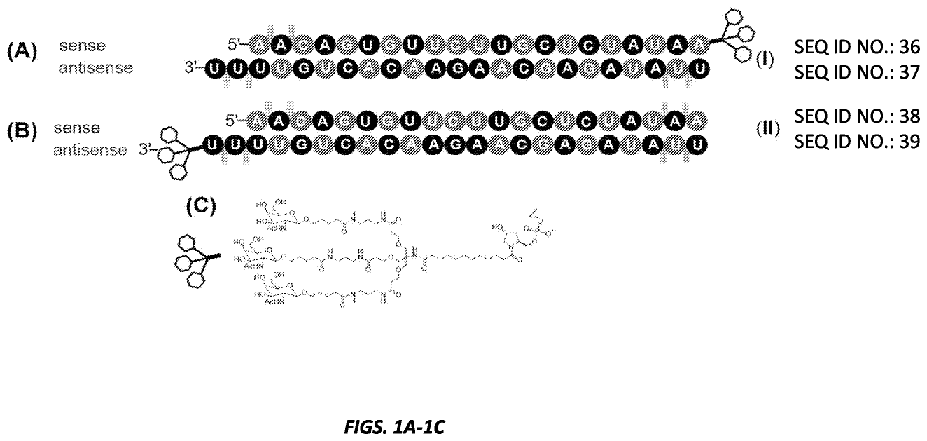

Therapeutics that act through the RNA interference (RNAi) pathway prevent production of disease-causing proteins (1-3). Synthetic small interfering RNA (siRNAs), which induce gene silencing via the endogenous RNAi process, are chemically modified to increase stability against nuclease degradation, to facilitate their cellular uptake through cell-membrane, and to reduce their immune stimulation (4,5). The first RNAi drug to be approved for clinical use was patisiran (ONPATTRO®), which is used to treat patients with polyneuropathy caused by hereditary ATTR amyloidosis. This siRNA is partially modified with 2′-O-methyl (2′-OMe) and encapsulated in lipid nanoparticles (6). A second RNAi therapeutic, givosiran (GIVLAARI®) has been approved for the treatment of acute hepatic porphyrias (7,8). More recently, both US FDA and EMA have approved a third RNAi drug, lumasiran (OXLUMO®) for the treatment of primary hyperoxaluria type 1 in all age groups (9), and EMA has approved the fourth RNAi drug, inclisiran (Leqvio®) for the treatment of adults with heterozygous familial hypercholesterolemia (10-12). These three compounds are fully modified with 2′-fluoro (2′-F) and 2′-O-methyl (2′-OMe) (FIG. 1) to confer stability in the absence of a lipid. These modifications are tolerated by the endonuclease silencer enzyme Argonaute-2 (Ago2), the catalytic component of the RNA-induced silencing complex (RISC) (13-17). The passenger strands of these siRNAs are conjugated with a tri-N-acetylgalactosamine (GalNAc; FIG. 1B), which interacts with the asialoglycoprotein receptor that is highly expressed on hepatocytes, to facilitate specific delivery into the liver (8,9,12,18).

Gene silencing activity of siRNA with GalNAc conjugated on either 3′-AS (II, IV and VI) or 3′-S(I, III and V) strand in the presence or absence of a transfection agent. Activity of siRNA under transfection conditions indicate intrinsic potency whereas free uptake represents ASGPR receptor mediated intercellular transport activity through GalNAc ligand. For 3′-AS GalNAc conjugation, effect of PS removal from the overhang was also evaluated. For all three targets, siRNA activity and potency is maintained for 3′-AS against 3′-S GalNAc conjugation. Removal of phosphorothioate from the overhang connecting GalNAc did not show any change in activity under in vitro conditions.

Among the four approved siRNA drugs till date from Alnylam Pharmaceuticals Inc (Onpattro, Givlaari, Oxlumo and Leqvio), except Onpattro ¹, all siRNA drugs are composed of trivalent GalNAc conjugation at 3′-end of sense strand ²⁻⁷. Vutrisiran is another 3′-sense GalNAc conjugate shown promising Phase 3 results in Helios-A study. (Ref). ALN-AGT for hypertension and ALN-HBV02 (Vir 2108) are other GalNAc conjugates which have shown promising PhaseI/II results.

### Materials and Methods

siRNA Synthesis

Sterling solvents and reagents for the ABI synthesizer, 500-Å CPG solid-supports, and 2′-deoxy, 2′-O-methyl (2′-O-Me), and 2′-deoxy-2′-fluoro (2′-F) phosphoramidites were all purchased from ChemGenes and used as received. Low-water content acetonitrile was purchased from EMD Chemicals. Oligonucleotides were synthesized on an ABI-394 DNA/RNA synthesizer. A solution of 0.25 M 5-(S-ethylthio)-1H-tetrazole in acetonitrile was used as the activator. The phosphoramidite solutions were prepared at concentrations of 0.15 M in anhydrous acetonitrile. The oxidizing reagent was 0.02 M I₂ in THF/pyridine/H₂O. N,N-Dimethyl-N′-(3-thioxo-3H-1,2,4-dithiazol-5-yl) methanimidamide, 0.1 M in pyridine, was used as the sulfurizing reagent. The detritylation reagent was 3% dichloroacetic acid in dichloromethane. After completion of the automated synthesis, the solid support was washed with 0.1 M piperidine in acetonitrile for 10 min, then washed with anhydrous acetonitrile and dried with argon. Oligonucleotides were manually deprotected using a mixture of 30% NH₄OH/absolute ethanol (3:1, v/v; 0.5 mL/umol of solid support) for 6 h at 55° C. Solvent containing oligonucleotide was collected by filtration and stored at −20° C. prior to purification.

The crude oligonucleotides were purified by anion-exchange HPLC on an AKTA Purifier-100 chromatography system using a AP-1 glass column (10×200 mm, Waters) custom-packed with the DNA TSK-Gel Super Q-5 PW support (TOSOH Bioscience). The desired product was purified to >85% using a linear gradient of 0.22 M to 0.42 M NaBr in 0.02 M sodium phosphate, pH 8.5/15% (v) acetonitrile over 120-150 min at room temperature and then desalted by size exclusion chromatography on an AKTA Prime chromatography system using an AP-2 glass column (20×300 mm, Waters) custom-packed with Sephadex G25 (GE Healthcare) eluted with sterile nuclease-free water.

Oligonucleotides were analyzed by ion-exchange HPLC using a Thermo DNAPac Pa200 analytical column (4×250 mm). Buffer A was 0.025 M Tris-HCl, 1 mM EDTA in 15% CH₃CN, pH 8, and buffer B was buffer A plus 1 M NaBr in 15% CH₃CN, pH 8. A gradient of 25 to 56% B over 21.5 min at a flow rate of 1.0 mL/min was used. The column temperature was 75° C. Oligonucleotides were also analyzed by LC/ESI-MS on a Waters XBridge C₈ column (2.1×50 mm, 2.5 μm). Buffer A was 95 mM 1,1,1,3,3,3-hexafluoro-2-propanol/16 mM triethylamine in water, and buffer B was 100% methanol. A gradient from 2% to 29% B over 26.8 min with flow rate of 0.25 mL/min was employed. The column temperature was 60° C. The oligonucleotide sequences and chemical modification and mass spectroscopy data are summarized in Table 1.

TABLE 1
RNA Oligonucleotides: Chemical modifications, sequence
information and analytical characterization

| | | | | SEQ | | | |
|---|---|---|---|---|---|---|---|
| | | S/AS | Sequence | ID | Chemis- | Mass | |
| Target^{[a]} | Duplex ID | (LN) | (5′-3′)^{[b]} | NO. | try^{c} | Calcd. | Found |
| mTTR | I 57727 | S | A•a•CaGuGuUCUuGc | 36 | A | 8590.17 | 8588.81 |
| (ESC)^{[c]} | | (21) | UcUaUaAL96 | | | | |
| | | AS | u•U•aUaGaGcAagaA | 37 | | 7595.94 | 7594.58 |
| | | (23) | cAcUgUu•u•u | | | | |
| | II 77197 | S | A•a•CaGuGuUCUuGc | 38 | B | 6833.34 | 6832.18 |
| | | (21) | UcUaU•a•A | | | | |
| | | AS | u•U•aUaGaGcAagaA | 39 | | 9384.90 | 9383.53 |
| | | (23) | cAcUgUu•u•uL96 | | | | |
| | VII 80301 | S | A•a•CaGuGuUCUuGc | 40 | C | 6833.34 | 6832.18 |
| | | (21) | UcUaU•a•A | | | | |
| | | AS | u•U•aUaGaGcAagaA | 41 | | 9352.77 | 9351.18 |
| | | (23) | cAcUgUuuuL96 | | | | |
| | VIII 155481 | S | A•a•CaGuGuUCUuGc | 42 | D | 8622.30 | 8621.28 |
| | | (21) | UcUaU•a•AL96 | | | | |
| | | AS | u•U•aUaGaGcAagaA | 43 | | 7595.94 | 7594.58 |
| | | (23) | cAcUgUu•u•u | | | | |
| | IX 155482 | S | A•a•CaGuGuUCUuGc | 44 | C | 8590.17 | 8588.81 |
| | | (21) | UcUaUaAL96 | | | | |
| | | AS | u•U•aUaGaGcAagaA | 45 | | 9320.77 | 9319.95 |
| | | (23) | cAcUgUudTdTL96 | | | | |
| | X 155483 | S | A•a•CaGuGuUCUuGc | 46 | B | 8590.17 | 8588.81 |
| | | (21) | UcUaUaAL96 | | | | |
| | | AS | u•U•aUaGaGcAagaA | 47 | | 9352.90 | 9352.00 |
| | | (23) | cAcUgUu•dT•dTL96 | | | | |
| | XI 157476 | S | a•A•aCaGuGuUCUuG | 48 | B | 7176.57 | |
| | | (22) | cUcUaU•a•A | | | | |
| | | AS | u•U•aUaGaGcAagaA | 49 | | 9064.71 | |
| | | (22) | cAcUgU•u•uL96 | | | | |
| | XII 157477 | S | a•A•aCaGuGuUCUuG | 50 | B | 7176.57 | |
| | | (22) | cUcUaU•a•A | | | | |
| | | AS | u•U•aUaGaGcAagaA | 51 | | 9384.90 | 9383.53 |
| | | (23) | cAcUgUu•u•uL96 | | | | |
| | XIII 192409 | S | A•a•CaGuGuUCUuGc | 52 | B | 6833.34 | 6832.18 |
| | | (21) | UcUaU•a•A | | | | |
| | | AS | u•U•aUaGaGcAagaA | 53 | | 8744.51 | |
| | | (21) | cAcUg•U•uL96 | | | | |
| | XIV 192410 | S | A•a•CaGuGuUCUuGc | 54 | B | 6833.34 | 6832.18 |
| | | (21) | UcUaU•a•A | | | | |
| | | AS | u•U•aUaGaGcAagaA | 55 | | 9064.71 | |
| | | (22) | cAcUgU•u•uL96 | | | | |
| | XV 77196 | S | A•a•CaGuGuUCUuGc | 56 | B | 6833.34 | 6832.18 |
| | | (21) | UcUaU•a•A | | | | |
| | | AS | u•U•aUaGaGcAagaA | 57 | | 9993.28 | 9991.56 |
| | | (25) | cAcUgUu•u•udTdTL | | | | |
| | | | 96 | | | | |
| | XVI80302 | S | A•a•CaGuGuUCUuGc | 58 | B | 6833.34 | 6832.18 |
| | | (21) | UcUaU•a•A | | | | |
| | | AS | u•U•aUaGaGcAagaA | 59 | | 9993.28 | 9991.73 |
| | | (25) | cAcUgUuuu•dT•dTL | | | | |
| | | | 96 | | | | |
| C5 | III 58641 | S | U•g•AcAaAaUAAcUc | 60 | A | 8627.29 | 8625.59 |
| (ESC) | | (21) | AcUaUaAL96 | | | | |
| | | AS | u•U•aUaGuGaGuuaU | 61 | | 7551.83 | 7550.53 |
| | | (23) | uUuGuCa•a•u | | | | |
| | IV 81865 | S(21) | U•g•AcAaAaUAAcUc | 62 | B | 6870.46 | 6869.46 |
| | | | AcUaU•a•A | | | | |
| | | AS | u•U•aUaGuGaGuuaU | 63 | | 9340.79 | 9339.39 |
| | | (23) | uUuGuCa•a•uL96 | | | | |
| | XVII 81866 | S | U•g•AcAaAaUAAcUc | 64 | C | 6870.46 | 6869.46 |
| | | (21) | AcUaU•a•A | | | | |
| | | AS | u•U•aUaGuGaGuuaU | 65 | | 9308.657 | — |
| | | (23) | uUuGuCaauL96 | | | | |
| FXII | VI 81867 | S | g•a•aacuCaAUAaag | 66 | B | 6999.73 | 6998.67 |
| (advanced | | (21) | ugcuu•u•a | | | | |
| ESC) | | AS | u•A•aagCacuuuauU | 67 | | 9399.00 | 9397.78 |
| | | (23) | gAguuuc•u•gL96 | | | | |
| | XVIII 81868 | S | g•a•aacuCaAUAaag | 68 | C | 6999.73 | 7608.39 |
| | | (21) | ugcuu•u•a | | | | |
| | | AS | u•A•aagCacuuuauU | 69 | | 9366.87 | 9365.62 |
| | | (23) | gAguuucugL96 | | | | |
| | V 74210 | S | g•a•aacuCaAUAaag | 70 | A | 8756.56 | 8755.62 |
| | | (21) | ugcuuuaL96 | | | | |
| | | AS | u•A•aagCacuuuauU | 71 | | 7610.04 | 7608.39 |
| | | (23) | gAguuuc•u•g | | | | |
| SS | XIX 319053 | AS | VPu•U•aU•aG•aG•c | 72 | | 9605.49 | 9604.05 |
| | | (23) | A•ag•aA•cA•cU•gU | | | | |
| | | | •u•u•uL96 | | | | |
| | XX 319054 | AS | VPu•U•au•aG•aG•C | 73 | | 9657.66 | 9655.78 |
| | | (23) | •aa•ga•A•cA•cu•g | | | | |
| | | | u•u•u•uL96 | | | | |

^{[a]}siRNA targeting mRNA encoding mouse transthyretin (mTTR)
^{[b]}S and AS indicate sense and antisense strands Italicized upper case and normal lower case letters indicate 2′-deoxy-2′-fluoro, and 2′-O-methyl sugar modifications, respectively, to adenosine (A), cytidine (C), guanosine (G) and uridine (U); dT indicate deoxythymidine;
•Indicate phosphorothioate (PS) linkage. (LN) = length of nucleotide.
^{[c]}ESC - Enhanced Stable Chemistry for providing improved metabolic stability (See, for example, WO2016/028649, content of which is incorporated herein by reference in its entirety), and advanced ESC - Enhanced Stable Chemistry for providing improved metabolic stability with off-target mitigation modification (see, for example, WO2018/098328 and WO2019/222479, contents of which are incorporated herein by reference in their entireties).

To generate siRNA duplexes, equimolar amounts of purified complementary strands were mixed to a final concentration of 20 μM in PBS, pH 7.4., heated in a water bath at 95° C. for 5 min, and cooled to room temperature over a period of approximately 12 h.

Analysis of Binding of siRNAs to ASGPR

Binding of siRNAs to ASGPR was evaluated using a previously described flow cytometry-based competitive binding assay.⁸ In brief, freshly isolated hepatocytes were resuspended at 1 million cells per mL in Dulbecco's Modified Eagle Medium (DMEM, Life Technologies) with 2% bovine serum albumin (BSA, Sigma-Aldrich). GalNAc3-conjugated, Alexa647-labeled siRNA described previously⁹ was diluted to a final concentration of 20 nM and was premixed with the siRNA to be evaluated at concentrations from 3 μM to 1.4 nM in 2% BSA in DMEM. To the siRNA solution was added 100,000 hepatocytes, and samples were incubated at 4° C. for 15 min. Cells were washed twice with 2% BSA in Dulbecco's Phosphate-Buffered Saline with magnesium and calcium (DPBS, Life Technologies). Cells were suspended in a solution of 2% BSA in DPBS with 2 μg/mL propidium iodide and analyzed on an LSRII flow cytometer instrument (BD Biosciences). Compensation was performed using Diva software (BD Biosciences). Hepatocytes were gated by size using forward scatter and side scatter, and dead cells stained with propidium iodide were excluded from analysis. Median fluorescent intensity of the GalNAc3-conjugated, Alexa647-labeled siRNA was quantified. Data were analyzed using FlowJo and GraphPad Prism.

### In Vitro Analysis of Gene Silencing

siRNAs were transfected into primary mouse hepatocytes or were analyzed after allowing free uptake. For transfection, 4.9 μl of Opti-MEM (Life Technologies), 0.1 μl of Lipofectamine RNAiMax (Invitrogen), and 5 μl of siRNA duplex were added to wells of a 384-well plate. After incubation at room temperature for 15 min, 40 μl of William's E Medium (Life Technologies) supplemented with 10% fetal bovine serum (Life Technologies) containing approximately 5×10³ cells were added to each well. Cells were incubated for 24 h, and then RNA was isolated. Free uptake experiments with primary mouse hepatocytes were performed by adding 5 μl of Opti-MEM to 5 μl of siRNA in wells of a 384-well plate. We then added approximately 5×10³ cells in 40 μl of William's E Medium with 10% fetal bovine serum (Life Technologies) to the siRNA. Cells were incubated for 48 h, and then RNA was isolated.

RNA was isolated using an automated protocol on a BioTek-EL406 platform using Dynabeads (Invitrogen). Briefly, 70 μl of Lysis/Binding Buffer and 10 μl of Lysis Buffer supplied with the Dynabeads containing 3 μl of magnetic beads were added to the plate with cells. Plates were incubated on an electromagnetic shaker for 10 min at room temperature, and then the magnetic beads were captured, and the supernatant was removed. Bead-bound RNA was then washed twice with 150 μl 10 mM Tris-HCl, pH 7.5, 150 mM LiCl, 1 mM EDTA, pH 8, and 0.10% lithium dodecyl sulfate and once with 10 mM Tris-HCl, pH 7.5, 150 mM LiCl, and 1 mM EDTA, pH 8. Beads were then washed with 150 μl 10 mM Tris-HCl, pH 7.5, re-captured, and supernatant removed. All buffers were purchased from Invitrogen.

The RNA of interest was quantified using RT-PCR. cDNA synthesis was performed from a 250-ng sample of RNA using the ABI High-capacity cDNA reverse transcription kit following the manufacturer's protocol. For each gene of interest, a pair of unlabeled PCR primers and a TaqMan probe were designed and synthesized. The probe was conjugated to VIC at the 5′ end and a minor groove binder, non-fluorescent quencher at the 3′ end. Target gene expression was normalized to Gapdh amplified in each well utilizing a dual-label system; the control probe targeting Gapdh was labeled with FAM. A 2 μl aliquot of cDNA was added to a master mix containing 0.5 μl of Gapdh, 0.5 μl target probe, and 5 μl Lightcycler 480 probe master mix (all from Roche) per well in a 384-well plate (Roche). Real-time PCR was performed in a LightCycler480 Real Time PCR system (Roche). Ct values were measured using a Roche Light Cycler 480. The following formula was used to determine relative gene expression: 2^{-(Ct Target)}/2^{-(Ct Control)}. Each experiment was performed at least twice. To calculate relative fold change, data were analyzed using the ΔΔCt method and normalized to data on cells transfected with a non-targeting control siRNA of the same chemistry. The following probes were used (all from ThermoFisher): GAPDH probe (4352339E), C5 probe (Mm00439275_m1), TTR probe (Mm00443267_m1), FXII probe (Mm00491349_m1), and CTNNB1 probe (Mm00483039_m1).

In Vivo Evaluation of siRNA-Mediated Silencing

In vivo experiments were conducted in 6-8 week-old female C₅₇BL/6 or Balb/c mice acquired from Charles River Laboratories. All studies were conducted at Alnylam Pharmaceuticals in accordance with animal procedures reviewed and approved by the Institutional Animal Care and Use Committee. Animals were administered siRNA or PBS (Gibco) via subcutaneous injection. Animals were sacrificed at time points ranging from 1 to 28 days post-dose.

Livers were harvested and snap frozen for analysis of the hepatic mRNA of interest. Total RNA was isolated using QIAzol reagent (Qiagen) or using the Qiagen RNAeasy kit. RNA concentrations were determined using a Nanodrop spectrophotometer (ThermoFisher Scientific). The RNA concentrations were adjusted to 25 ng/μl, and cDNA was synthesized from 250 ng of sample using a reverse transcription kit from Applied Biosystems. RT-PCR was employed for RNA quantification as described in the section on analysis of in vitro activity.

Blood was collected utilizing the retro-orbital eye bleed procedure at selected time points to assess levels of proteins of interest. For this procedure, the mice were anesthetized using isoflurane. Heparin-coated capillary tubes (Fisher Scientific) were inserted into the posterior corner of the mouse eye; the tube was inserted at a 45-degree angle to approximately 1 cm and rotated until the blood from the retro-orbital sinus was released. Approximately 200 μl was collected from the left eye of each mouse according to the IACUC protocol for blood collection. The blood was collected in Becton Dickinson (BD) serum separator tubes. Serum samples obtained for analysis of proteins other than C₅ were kept at room temperature for 1 h and then spun in a microcentrifuge at 22× g at room temperature for 10 minutes. Serum was transferred to 1.5-ml microcentrifuge tubes for storage at −80° C. until samples were processed.

For analysis of serum TTR levels, serum samples were diluted 1:4000 and assayed using an ELISA (ALPCO, catalog number 41-PALMS-E01). Protein concentrations were determined by comparison to a TTR standard prepared in-house.

Evaluation of In Vivo RISC Loading and Liver Levels of siRNA

In vivo RISC loading was evaluated according to a published procedure.¹⁰ Ago2-bound siRNA from mouse liver was quantified by preparing liver powder lysates at 100 mg/mL in 50 mM Tris-HCl, pH 7.5, 150 mM NaCl, 2 mM EDTA, 0.5% Triton-X 100 supplemented with freshly added protease inhibitors (Sigma-Aldrich, P8340) at 1:100 dilution and 1 mM PMSF (Life Technologies). Total liver lysate (10 mg) was used for each Ago2 immunoprecipitation (IP) and control IP. Anti-Ago2 antibody was purchased from Wako Chemicals (Clone No.: 2D4). Control mouse IgG was from Santa Cruz Biotechnology (sc-2025). Protein G Dynabeads (Life Technologies) were used to precipitate antibodies. Ago2-associated siRNAs were eluted by heating in 50 μL PBS, 0.25% Triton (95° C., 5 min) and quantified by stem-loop RT-qPCR as described previously.¹¹⁻¹²

Determination of liver levels of siRNA-GalNAc conjugates were performed according to a previously published procedure.¹³ Mice were sacrificed on day 5 (mTTR and C₅ experiments) or day 9 (FXII experiment) post-dose, and livers were snap frozen in liquid nitrogen and ground into powder. Total siRNA liver levels were measured by reconstituting liver powder at 10 mg/mL in PBS containing 0.25% Triton-X 100. The tissue suspension was ground with 5-mm steel grinding balls at 50 cycles/second for 5 min in a tissue homogenizer (Qiagen TissueLyser LT) at 4° C. Homogenized samples were then heated at 95° C. for 5 min, briefly vortexed, and allowed to rest on ice for 5 min. Samples were then centrifuged at 21,000×g for 5 min at 4° C. The siRNA-containing supernatants were transferred to new tubes. siRNA sense and guide strand levels were quantified by stem-loop RT-qPCR.¹¹⁻¹²

### Results

GalNAc Conjugation to the 3′ End of the siRNA Antisense Strand does not Impair Silencing in Vitro

siRNAs targeting mouse TTR were prepared with GalNAc conjugated at the 3′ termini of the sense strands, the typical design, or with the GalNAc conjugated to the 3′ termini the antisense strands (FIG. 1 and Table X). The siRNAs with sense strand conjugation contained six phosphorothioate linkages and were modified with chemical modifications as described earlier.⁹ The second set of siRNAs were conjugated at the 3′ end of the antisense strand to GalNAc, contained eight phosphorothioate linkages, and were modified with same chemistries.

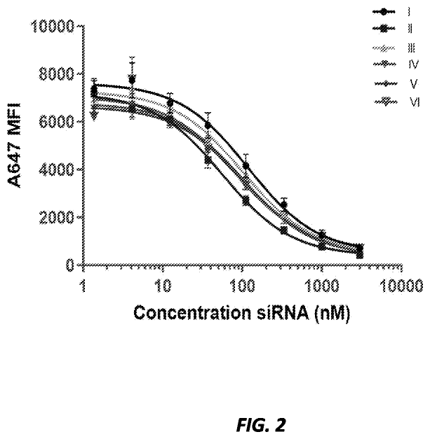

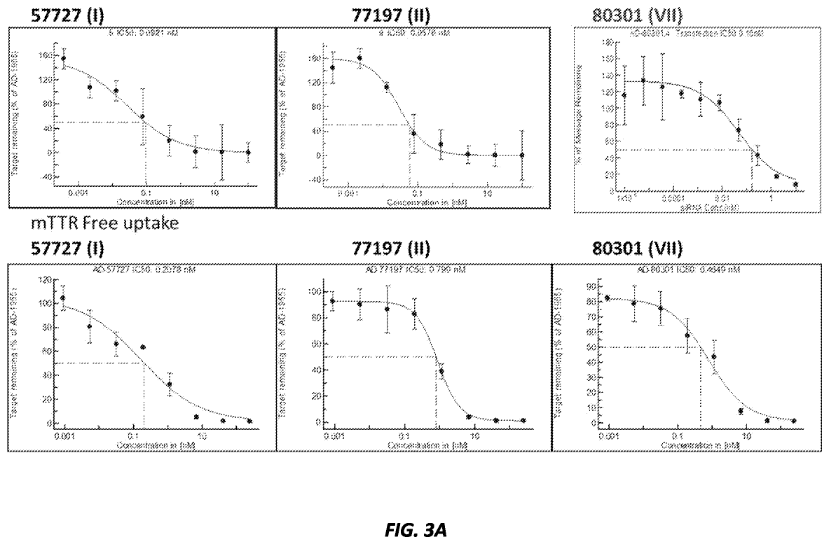

The affinities of these siRNAs for a complementary oligoribonucleotide were determined using a fluorescence-based assay. siRNAs with the triantennary GalNAc unit conjugated to the 3′ end of antisense strand had affinities for ASGPR similar to that of siRNAs in which GalNAc was conjugated to the 3′ end of sense strand (FIG. 2).

The ASGPR binding affinities of the conjugated GalNAc moieties to siRNA were determined using a fluorescence-based assay.⁸ The siRNAs with GalNAc conjugated to the 3′ end of antisense strand had affinities for ASGPR similar to that of the parent designs in which GalNAc was conjugated to the 3′ end of sense strand (FIG. 2 and Table 2).

TABLE 2
ASGPR binding affinities of the conjugated
GalNAc moieties to siRNA

| Duplex | KI(nM) | Std. deviation |
|---|---|---|
| I | 51.1 | 8.6 |
| II | 24.1 | 2.7 |
| III | 43.8 | 4.8 |
| IV | 44.0 | 4.9 |
| V | 42.9 | 5.0 |
| VI | 34.7 | 9.7 |

Silencing of TTR expression was evaluated in primary mouse hepatocytes in the presence or absence of a transfection agent. Activity of siRNA under transfection conditions indicates intrinsic potency, whereas that under free-uptake conditions is also a measure of ASGPR-mediated internalization. For siRNAs conjugated to GalNAc at the 3′ end of the antisense strand, the effect of the phosphorothioate linkages in the overhang were also evaluated (Table 3). For TTR-targeted siRNAs, analysis of TTR mRNA showed no significant differences in activity for siRNAs with the GalNAc ligand on the 3′ end of the sense strand versus the antisense strand (Table 3). Removal of phosphorothioate from the overhang connecting GalNAc to the 3′ end of the antisense strand was not detrimental under in vitro conditions (Table 3).

TABLE 3
siRNAs targeting TTR, affinity for ASGPR,
and in vitro silencing.

| | | | | | IC₅₀ [nm]^{[d]} | |
|---|---|---|---|---|---|---|
| | | | SEQ | | Trans- | Free |
| Duplex | S/AS | Sequence | ID | | fec- | up- |
| ID | (length)^{[a]} | 3(5′-′)^{[b]} | NO. | Kᵢ[nm]^{[c]} | tion | take |
| I | S (21) | A•a•CaGuGuUC | 74 | 51.1 ± 8.6 | 0.0451 | 0.1023 |
| | | UuGcUcUaUaA-L96 | | | | |
| | AS (23) | u•U•aUaGaGcA | 75 | | | |
| | | agaAcAcUgUu•u•u | | | | |
| II | S (21) | A•a•CaGuGuUC | 76 | 24.1 ± 2.7 | 0.0368 | 0.1074 |
| | | UuGcUcUaU•a•A | | | | |
| | AS (23) | u•U•aUaGaGcA | 77 | | | |
| | | agaAcAcUgUu•u•u-L96 | | | | |
| VII | S (21) | A•a•CaGuGuUC | 78 | n.d. | 0.0497 | 0.1414 |
| | | UuGcUcUaU•a•A | | | | |
| | AS (23) | u•U•aUaGaGcA | 79 | | | |
| | | agaAcAcUgUuuu-L96 | | | | |
| VIII | S (21) | A•a•CaGuGuUC | 80 | n.d. | 0.162 | 0.134 |
| | | UuGcUcUaU•a•A | | | | |
| | AS (23) | u•U•aUaGaGcA | 81 | | | |
| | | agaAcAcUgUu•u•u | | | | |
| IX | S (21) | A•a•CaGuGuUC | 82 | n.d. | 0.069 | 0.286 |
| | | UuGcUcUaU•a•A | | | | |
| | AS (23) | u•U•aUaGaGcA | 83 | | | |
| | | agaAcAcUgUudTdT-L96 | | | | |
| X | S (21) | A•a•CaGuGuUC | 84 | n.d. | 0.072 | 0.139 |
| | | UuGcUcUaU•a•A | | | | |
| | AS (23) | u•U•aUaGaGcA | 85 | | | |
| | | agaAcAcUgUu•dT•dT-L96 | | | | |
| XV | S (21) | A•a•CaGuGuUC | 86 | n.d. | | |
| | | UuGcUcUaU•a•A | | | | |
| | AS (25) | u•U•aUaGaGcA | 87 | | | |
| | | agaAcAcUgUu•u•udTdT-L96 | | | | |
| XVI | S (21) | A•a•CaGuGuUC | 88 | n.d. | | |
| | | UuGcUcUaU•a•A | | | | |
| | AS (25) | u•U•aUaGaGcA | 89 | | | |
| | | agaAcAcUgUuuu•dT•dT-L96 | | | | |

^{[a]}S and AS indicate sense and antisense strands. Length in nucleotides of each strand is given in parentheses.
^{[b]}Italicized upper case and normal lower case letters indicate 2′-deoxy-2′-fluoro, and 2′-O-methyl sugar modifications, respectively, to adenosine (A), cytidine (C), guanosine (G) and uridine (U); dT indicates deoxythymidine;
•indicates phosphorothioate linkage; L96 indicates GalNAc.
^{[c]}Binding affinity to ASGPR (on mouse primary hepatocytes, fluorescent based assay).
^{[d]}Half maximal inhibitory concentration (IC₅₀) for in vitro gene silencing under transfection (Lipofectamine RNAiMAX) or free uptake conditions in primary mouse hepatocytes.

To confirm that this finding was broadly relevant, silencing in vitro by siRNAs targeting C5 and FXII was evaluated. For all three targets, siRNA activity and potency was maintained despite conjugation of the GalNAc ligand to the 3′ end of the antisense strand. Results are shown in FIGS. 3A-3C and summarized in Table 4.

TABLE 4
IC₅₀ (nM)

| | mTTR (ESC) | | | C5 (ESC) | | | FXII (advanced ESC) | |
|---|---|---|---|---|---|---|---|---|
| Duplex | | Free | Duplex | | Free | Duplex | | Free |
| No. | Transfection | Uptake | No. | Transfection | Uptake | No. | Transfection | Uptake |
| I | 0.0087 | 0.208 | III | 0.488 | 0.341 | V | 0.972 | 0.881 |
| II | 0.0074 | 0.799 | IV | 0.304 | 0.113 | VI | 0.937 | 0.776 |
| VII | 0.0090 | 0.465 | XVII | 0.362 | 0.102 | XVIII | 0.821 | 4.169 |

In Vivo Activity of siRNA is not Impaired by GalNAc Conjugation to the Antisense Strand

It was previously demonstrated that the siRNA with GalNAc conjugated to 3′ end of the sense strand of the siRNA (I) significantly reduces levels of circulating TTR protein in C57BL/6 mice.¹⁴ To evaluate the 3′-antisense conjugation, mice were treated subcutaneously with the parent siRNA (I) at 1 mg/kg and with the 3′-antisense conjugate (II) at 0.5 and 1 mg/kg. Levels of circulating TTR protein were analyzed after 96 and 168 hours. The designs resulted in comparable reductions in TTR at the 1 mg/kg dose (FIG. 4). Furthermore, there was a greater reduction in TTR levels at both time points when mice were treated with 1 mg/kg than with 0.5 mg/kg of II.

### In Vivo Activity Depends on Phosphorothioate Linkages and Length of the Antisense Strand

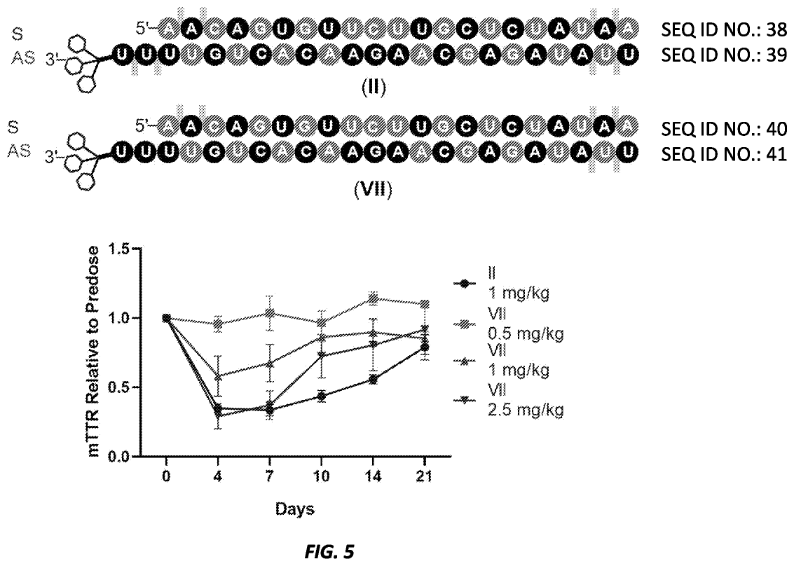

In the parent design with the GalNAc at the 3′ end of the sense strand, there are six phosphorothioate linkages. In the design used for the 3′-antisense-strand conjugate, there are eight phosphorothioate linkages as two phosphorothioates were introduced at the 3′ end of the sense strand to protect that terminus from nucleases. The GalNAc is conjugated to the oligonucleotide through a trans-4-hydroxyprolinol linker.⁹ Without wishing to be bound by a theory, the linker and the bulky ligand may provide enough nuclease protection that presence of the two phosphorothioates are not needed at the 3′ end of the antisense strand. To analyze this, mice were treated with siRNA II, which has two phosphorothioate linkages at the 3′ end of the antisense strand, and with siRNA VII, which has phosphodiester bonds at these positions. The siRNAs were administrated subcutaneously, and levels of circulating TTR protein were analyzed 4, 7, 10, 14, and 21 days post-dose. Results are shown in FIG. 5. At 1 mg/kg, the siRNA with eight phosphorothioate linkages (II) was more efficacious than the siRNA with six phosphorothioate linkages: the maximum reduction in TTR was 75% for II but only 40% for VII. The siRNA with eight phosphorothioate linkages also had longer duration of action (FIG. 5). At the highest dose evaluated, siRNA VII had high activity (75% reduction at 96 hours), but its activity had returned close to baseline within 10 days. At 10 days post-dose, levels of TTR were reduced by approximately 60% compared to the control by a 1 mg/kg dose of II. Thus, phosphorothioate linkages are required at the 3′ end of the antisense strand in this design. This effect was not observed in vitro conditions, most likely due to a more favorable nuclease environment.

In the siRNAs comprising sense strand of length 21 nucleotides and antisense strand of length of 23 nucleotides, incorporation of the phosphorothioate linkages at the 3′-end of the antisense improved the potency and duration of activity. As seen from FIG. 5, siRNA II (8 phosohorothioate linkages) out-performed siRNA VII (6 phosohorothioate linkages).

### In Vivo Evaluation of 3′-Sense to 3′-Antisense GalNAc Conjugation: 6 PS and 8 PS Designs

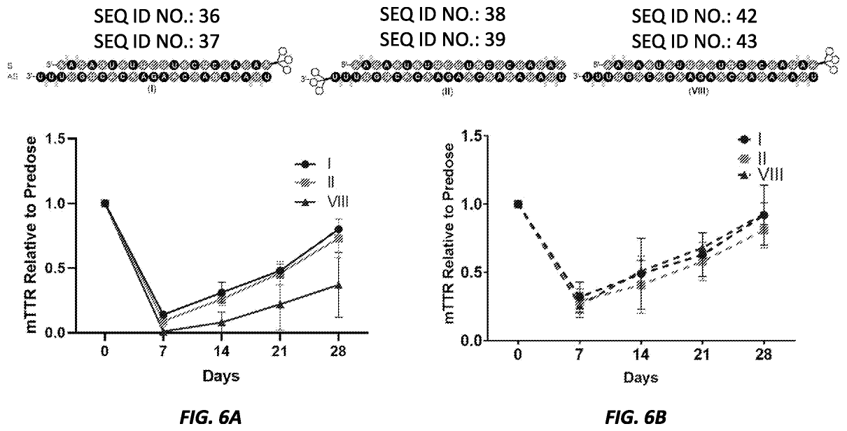

As seen from the data shown in FIGS. 6A and 6B, siRNAs comprising GalNAc comprising 8 phosphorothioates and GalNAc linked to the 3′-end of the sense strand (VIII) or 3′-end of the antisense strand (II) showed equivalent potency.

Effect of DNA Guide Strand (with or without PS) on Overhang: (21/23 with Two Different Chemistries: DNA and 2′-OMe)

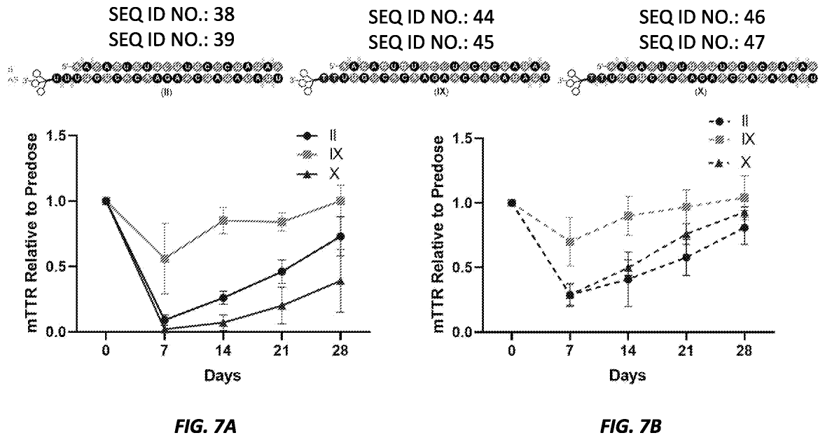

The activity of siRNAs comprising different chemistries in the overhang were evaluated. The 2′-OMe-uridines in in the overhang were replaced by their DNA counterpart, i.e., 2′-deoxythymidines. If DNA is not stabilized by phosphorothioate backbone, it can be susceptible to nuclease degradation. The increased length was also evaluated. In vivo activities of siRNA IX, DNA overhang with phosphate linkage, and siRNA (X), DNA overhang with phosphorothioate linkage, were studied. siRNA (II) was used as control. All three siRNA (II, IX and X) were dosed to wild type C₅₇BL/6 mice for mouse transthyretin mRNA (Ttr) through single subcutaneous administration at 1 and 2.5 mg/kg dose and levels of circulating TTR protein were analyzed after 168, 336, 504 and 672 h post-dose. Results are shown in FIGS. 7A and 7B. At low (1 mg/kg) dose siRNA (II) and (X) showed similar efficacy and duration. Maximum 75% suppression was observed at day 7 and activity of both siRNA's were recovered to baseline by 28 days post-dose. siRNA (XI) was found out to undergo a loss in potency and duration compared to the other two siRNA used in this experiment. Maximum 30% suppression was observed on day 7 and the activity has returned to baseline on day 14. This loss in activity may be attributed to metabolic liability arising from not having phosphorothioate on overhang, which is exposed single strand. At higher dose (2.5 mg/kg) siRNA (XI) had 40% suppression on day 7 and the activity has returned to baseline on day 14. siRNA (X) showed improved duration (60% suppression on day 28) than siRNA (II) (25% suppression).

Data in FIGS. 7A and 7B shows:
At low dose (1 mg/kg) duplexes with PS stabilized overhang (II and X) have comparable activity and duration.
If DNA overhang is not stabilized with PS then the duplex (IX) does undergo a loss in potency and duration.
At high dose (2.5 mg/kg) X shows improved duration profile compared to II.
Most robust and durable PD response observed with SQ administration of X at 2.5 mg/kg (max of >90% TTR suppression)·
Serum TTR KD observed in comparator groups
Extra OMe and PS modification (XVI) achieved 80% TTR suppression
DNA w/out PS (IX) achieved ˜40% TTR suppression
PS modification of DNA bases improved duration relative to control and other modifications.
Max suppression by Day 7 with recovery beginning between 14 and 21 days post-dose but still at 60% suppression at day 28
TTR recovery to baseline observed with XVI and IX conjugates by day 28

The modifications in the overhang may not be required if the duplex comprises at least 8 phosphorothioates.

Silencing Activity Correlates Well with In Vivo Liver Exposure and RISC Loading of the Antisense Strand in Liver of siRNA Antisense Strand

Liver level determination of siRNA-GalNAc conjugates were done according to previously published procedure.¹³ Mice were sacrificed on day 5 (mTTR and C5)-post-dose, and livers were snap frozen in liquid nitrogen and ground into powder for further analysis. In-vivo RISC loading was done according to a published procedure¹⁰.

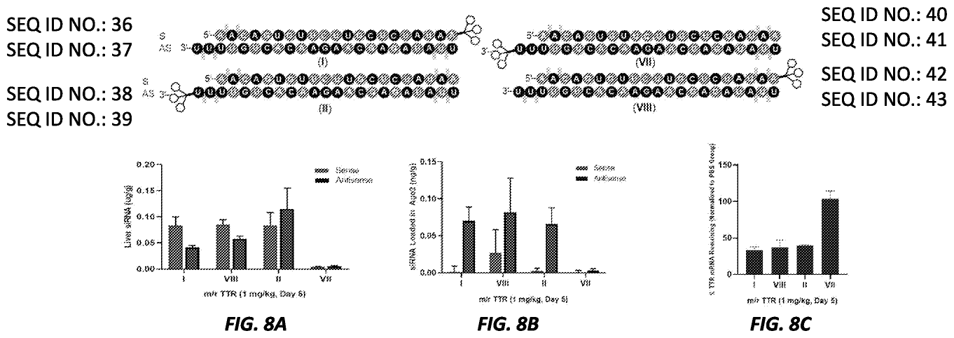

Ago2 associates with the 5′ end of the antisense strand via its MID domain and with the 3′ end of the antisense strand through the PAZ domain. Alterations in the structure of either of these ends can affect loading and subsequent gene silencing efficiency. We examined the impact of 3′-antisense GalNAc and other siRNA variants on liver stability and their ability to be loaded into Ago2 and form functional RISC complexes. There was comparable loading of the antisense strands of 1, 4, and 2, whereas the level of antisense strand of 3 loaded into RISC was low (FIG. 8B). This finding correlated with the observed decrease in liver Ttr mRNA levels: At 5 days after dosing with 1, 4, and 2, levels of Ttr mRNA in liver were reduced by 67%, 63%, and 61% compared to levels in mice treated with PBS (FIG. 8C). Interestingly, siRNA (3) did not show any mRNA knockdown in liver on day 5. Furthermore, there was a very low level of siRNA 3 present in liver (FIG. 8A). Levels of antisense strands of 1, 4, and 2 were comparable (FIG. 8A).

Wild-type C₅₇BL/6 mice (n=3) were treated with GalNAc-siRNA at 1 mg/kg single subcutaneous dose. The siRNAs used for this study are shown in FIG. 8A and the results are shown in FIGS. 8B-8D. After 5 days, siRNA (I), (VIII) and (II) consecutively showed 67%, 63% and 61% knockdown of TTR mRNA in liver. Interestingly, siRNA (VII) did not show any mRNA knockdown in liver on day 5. This is supported by very low level of siRNA (VII) present in liver. Overall level of antisense strand present in liver was comparable for siRNA (I), (VIII) and (II) but the level was much lower for (VII). This indicates the lower activity observed for (VII). In vivo Ago2 loading of siRNA was determined according to a published procedure.¹⁰ Similar to previous finding, siRNA (I), (VIII) and (II) had comparable Ago2 loaded antisense strand whereas (VII) had low Ago2 loading. These findings support individual observed activity of siRNAs. [Data: Gene Expression RES17-047 M_qPCR Analysis_09272017]

In short, the overhang prefers PS over PO and 8 PS construct outperforms 6 PS construct in vivo.

Effect of Architecture of siRNA Duplex with 3′-AS-Conjugates Using mTTR Constructs

### Effect of Length Variation in TTR Silencing Activity

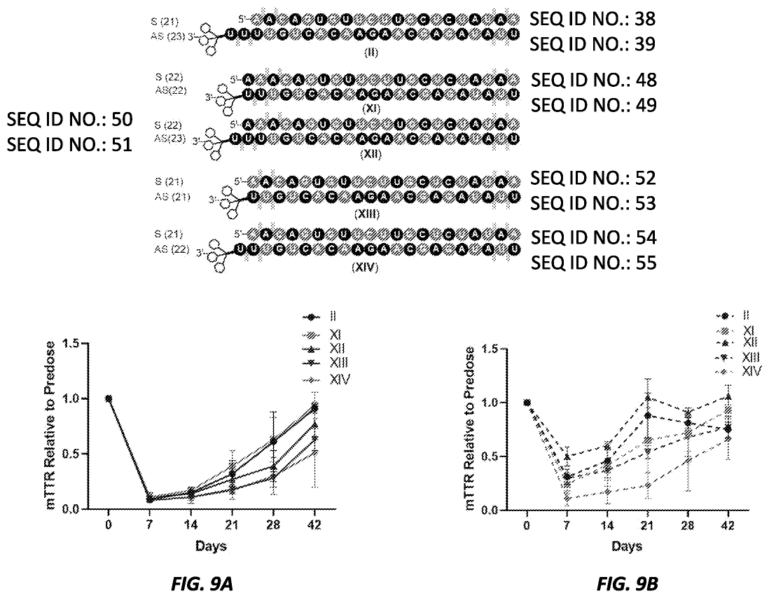

This study was undertaken to better understand PAZ interaction of guide strand (antisense strand). Standard 23-mer guide strand having GalNAc on 3′-S has 22-mer as an active in vivo metabolite. If these 21- and 22-mer guide strands are active, then this study can facilitate better insight of metabolic stability of antisense constructs. Results are shown in FIGS. 9A and 9B and summarized in Table 5. The results show that
Evaluation of architecture shows 21/22 (XIV) as the best construct
Compared to (II) 21/23, (XIV) 21/22 is better and even slightly better than (XIII) (may be due effective loading)
(XIV) show even longer duration of action compared to (II) and (XIII): 42 Day Study
(XIV) shows better efficacy and longer duration of action compared to (II), (XIII), (XI) and (XII): 42 day study

TABLE 5

| | | | | | | IC₅₀ [nm]^{e} | |
|---|---|---|---|---|---|---|---|
| | | | | SEQ | | Trans- | Free |
| | Duplex | S/AS | Sequence | ID | Kᵢ | fec- | up- |
| Target^{[a]} | ID | (LN) | (5′-3′)^{[b]} | NO. | [nm]^{d} | tion | take |
| mTTR | XI | S(22) | a•A•aCaGuGuU | 90 | n.d. | 0.068 | 0.116 |
| (ESC)^{[f]} | 157476 | | CUuGcUcUaU•a•A | | | | |
| | | AS(22) | u•U•aUaGaGcA | 91 | | | |
| | | | agaAcAcUgU•u•uL96 | | | | |
| | XII | S(22) | a•A•aCaGuGuU | 92 | n.d. | 0.042 | 0.045 |
| | 157477 | | CUuGcUcUaU•a•A | | | | |
| | | AS(23) | u•U•aUaGaGcA | 93 | | | |
| | | | agaAcAcUgUu•u•uL96 | | | | |
| | XIII | S(21) | A•a•CaGuGuUC | 94 | n.d. | 0.015 | 0.059 |
| | 192409 | | UuGcUcUaU•a•A | | | | |
| | | AS(21) | u•U•aUaGaGcA | 95 | | | |
| | | | agaAcAcUg•U•uL96 | | | | |
| | XIV | S(21) | A•a•CaGuGuUC | 96 | n.d. | 0.108 | 0.250 |
| | 192410 | | UuGcUcUaU•a•A | | | | |
| | | AS(22) | u•U•aUaGaGcA | | | | |
| | | | agaAcAcUg•u•uL96 | | | | |

^{[a]}siRNA targeting mRNA encoding mouse transthyretin (mTTR
^{[b]}S and AS indicate sense and antisense strands Italicized upper case and normal lower case letters indicate 2′-deoxy-2′-fluoro, and 2′-O-methyl sugar modifications, respectively, to adenosine (A), cytidine (C), guanosine (G) and uridine (U); and dT indicate deoxythymidine;
•Indicate phosphorothioate (PS) linkage.
^{d}Binding affinity to ASGPR (on mouse primary hepatocytes, fluorescent based assay).
^{e}Half maximal inhibitory concentration (IC₅₀) for in vitro gene silencing under transfection (Lipofectamine RNAiMAX) or free uptake conditions in primary mouse hepatocytes (LN) = length of nucleotide.
^{[f]}ESC - Enhanced Stable Chemistry for providing improved metabolic stability (See, for example, WO2016/028649, content of which is incorporated herein by reference in its entirety), and advanced ESC - Enhanced Stable Chemistry for providing improved metabolic stability with off-target mitigation modification (see, for example, WO2018/098328
and WO2019/222479, contents of which are incorporated herein by reference in their entireties).

### Increasing the Antisense Length Compromises Activity

Next, the antisense length was increased from 23 to 25 nucleotides, with a 4 nucleotide overhang to evaluate the possibility of using GalNAc end using a cleavable nucleotide string as a prodrug. Without wishing to be bound by a theory, the increased nucleotide length can function as a prodrug, and if there were steric clash with the linker, ligand and PAZ, the free antisense can be released after the delivery of conjugate. The results showed that
3′-end of antisense conjugate display silencing, but less efficiently than II even though 8 PS are present.
In this 21/25 configuration, cleavable linker is slightly less active and not really helping much (GalNAc may be even lost before reacting tissue) in a overhang phosphodiester nucleotide construct
Placing the PS at the 24/25 does not help because the cleavage is still possible around 22 and 23. Similarly PS in 22/23 alone is also not sufficient
Lack of the PS may be compromising the activity
Overhang stability seems to be important

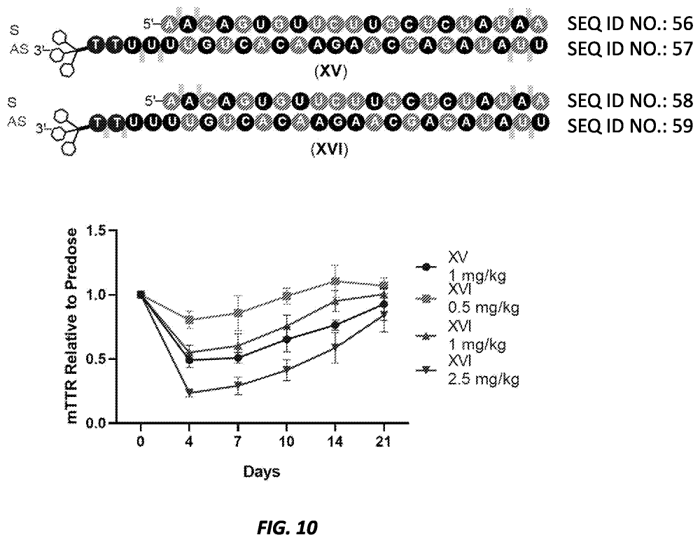

The position of the phosphorothioate residues relative to the terminal base pair of the siRNA duplex were varied next. Two siRNAs with four nucleotide overhangs were synthesized (FIG. 10). In siRNA XV, the two phosphorothioates are positioned similarly to those in siRNA II adjacent to the duplex and two dT nucleotides were introduced to extend the length of the overhand. In siRNA XVI, phosphorothioates link the two 3′-most nucleotides. The efficacy of Ttr silencing by siRNAs II and XVI were compared to that of XV in C57BL/6 mice. The siRNAs with the longer overhangs of the 3′ end of the antisense strand were less efficacious than siRNA II.

In Vivo Evaluation of 3′-Antisense GalNAc Conjugated C-5 Target siRNAs with 8 PS and 6 PS

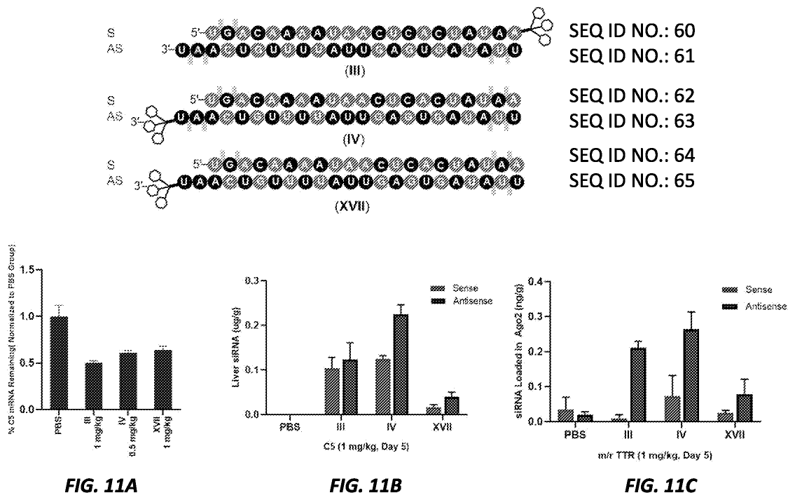

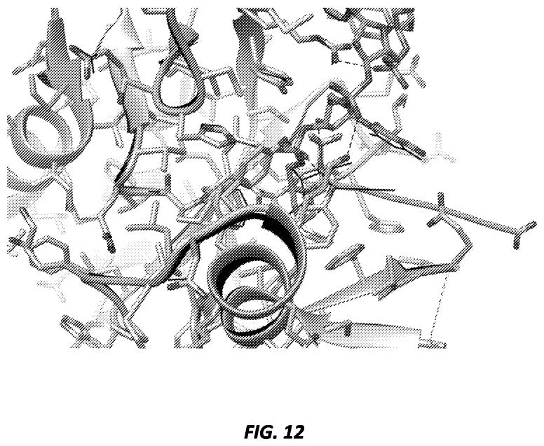

For mTTR target, the activity of 3′-AS GalNAc conjugated siRNA depends on overhang stability. Activity of siRNA with same motif but targeting a different target, C5, was evaluated. 3′-AS GalNAc conjugated siRNA IV with PS and (XVII) without PS on overhang was dosed to wild type C57BL/6 mice for mouse transthyretin mRNA (mTTR) through single subcutaneous administration at 1.0 mg/kg dose. Their activity was compared against siRNA (III) with 3′-S GalNAc conjugation. Levels of circulating C5 protein were analyzed after 120 h post-dose. Results are shown in FIG. 11A. 3′-S conjugated siRNA (III) showed 50% knockdown whereas 40% knockdown was observed for IV. Interestingly, unlike mTTR siRNA, detrimental effect from removal of PS from the overhang was not seen for C5 siRNA (XVII) showing 36% knockdown. It could be possible that the effect of metabolic instability may be seen at longer duration, which was not covered under experimental snapshot.

Wild-type C57BL/6 mice (n=3) were treated with GalNAc-siRNA at 1 mg/kg single subcutaneous dose. After 5 days, siRNA (III), (IV) and (XVII) consecutively showed 50%, 40% and 36% knockdown of circulating C₅ protein (FIG. 11A). siRNA (III) with 3′-S GalNAc conjugation and 6PS construct showed 0.13 μg/g antisense loading whereas siRNA (IV) with 3′-AS GalNAc conjugation and 8PS construct showed 0.22 μg/g antisense strand present in liver (FIG. 11B). Sense strand loading was comparable for both siRNAs (0.10 and 0.12 μg/g for III and IV). Overall level of sense (0.02 μg/g) and antisense (0.04 μg/g) present in liver was much lower for siRNA (XVII) (FIG. 11B).

As seen from FIG. 11C siRNA (III), and (IV) had comparable Ago2 loaded antisense strand in liver (0.21 and 0.26 ng/g respectively) whereas (XVII) had low Ago2 antisense loading (0.08 ng/g). Observed Ago2 loading is concurring with in vivo gene expression for III and IV. Lower level of siRNA present in liver and lower Ago2 loading of (XVII) may be observed in duration study which is missing in this experiment.
Activity appears to be retained with the GalNAc conjugated to the 3′AS of the C₅ (ESC) sequence
Observed Ago2 loading is concurring with in vivo gene expression for III and IV
Lower level of siRNA present in liver and lower Ago2 loading of XVII can be observed in duration study.

TABLE 6

| | | Se- | | | IC₅₀ [nm]^{e} | |
|---|---|---|---|---|---|---|
| | Du- | quence | SEQ | | Trans- | Free |
| Tar- | plex | (5′- | ID | Kᵢ | fec- | up- |
| get^{[a]} | ID | 3′)^{[b]} | NO. | [nm]^{d} | tion | take |
| C5 | III | U•g•Ac | 98 | 43.8 ± | 0.1129 | 0.130 |
| (ESC)^{[f]} | 58641 | AaAaUA | | 4.8 | | |
| | | AcUcAc | | | | |
| | | UaUaAL | | | | |
| | | 96 | | | | |
| | | u•U•aU | 99 | | | |
| | | aGuGaG | | | | |
| | | uuaUuU | | | | |
| | | uGuCa• | | | | |
| | | a•u | | | | |
| | IV | U•g•Ac | 100 | 44.0 ± | 0.0358 | 0.116 |
| | 81865 | AaAaUA | | 4.9 | | |
| | | AcUcAc | | | | |
| | | UaU•a• | | | | |
| | | A | | | | |
| | | u•U•aU | 101 | | | |
| | | aGuGaG | | | | |
| | | uuaUuU | | | | |
| | | uGuCa• | | | | |
| | | a•uL96 | | | | |
| | XVII | U•g•Ac | 102 | n.d. | 0.0794 | 0.127 |
| | 81866 | AaAaUA | | | | |
| | | AcUcAc | | | | |
| | | UaU•a• | | | | |
| | | A | | | | |
| | | u•U•aU | 103 | | | |
| | | aGuGaG | | | | |
| | | uuaUuU | | | | |
| | | uGuCaa | | | | |
| | | uL96 | | | | |

^{[a]}siRNA targeting mRNA encoding C5.
^{[b]}S and AS indicate sense and antisense strands Italicized upper case and normal lower case letters indicate 2′-deoxy-2′-fluoro, and 2′-O-methyl sugar modifications, respectively, to adenosine (A), cytidine (C), guanosine (G) and uridine (U); and dT indicate deoxythymidine;
•Indicate phosphorothioate (PS) linkage.
^{d}Binding affinity to ASGPR (on mouse primary hepatocytes, fluorescent based assay).
^{e}Half maximal inhibitory concentration (IC₅₀) for in vitro gene silencing under transfection (Lipofectamine RNAiMAX) or free uptake conditions in primary mouse hepatocytes (LN) = length of nucleotide.
^{e}Half maximal inhibitory concentration (IC₅₀) for in vitro gene silencing under transfection (Lipofectamine RNAiMAX) or free uptake conditions in primary mouse hepatocytes (LN) = length of nucleotide.
^{[f]}ESC - Enhanced Stable Chemistry for providing improved metabolic stability (See, for example, WO2016/028649, content of which is incorporated herein by reference in its entirety), and advanced ESC - Enhanced Stable Chemistry for providing improved metabolic stability with off-target mitigation modification (see, for example, WO2018/098328
and WO2019/222479, contents of which are incorporated herein by reference in their entireties).

Single-Stranded siRNA Conjugutes do not Function Effectively Even in High Doses

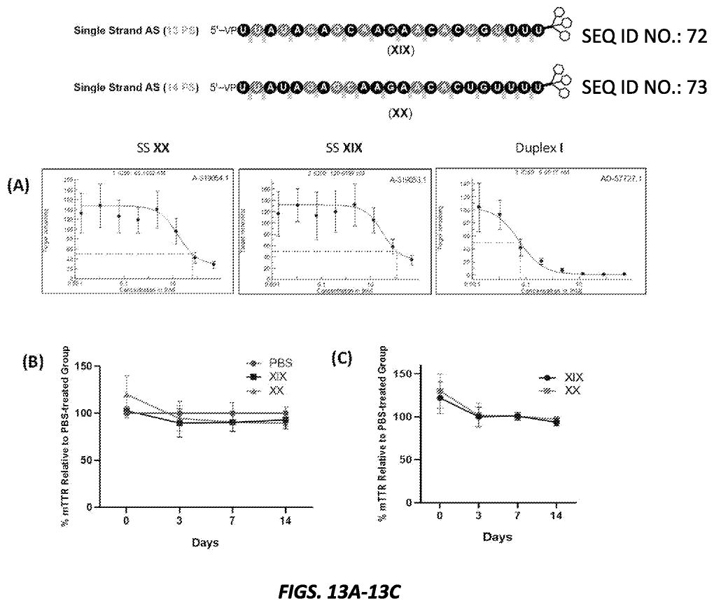

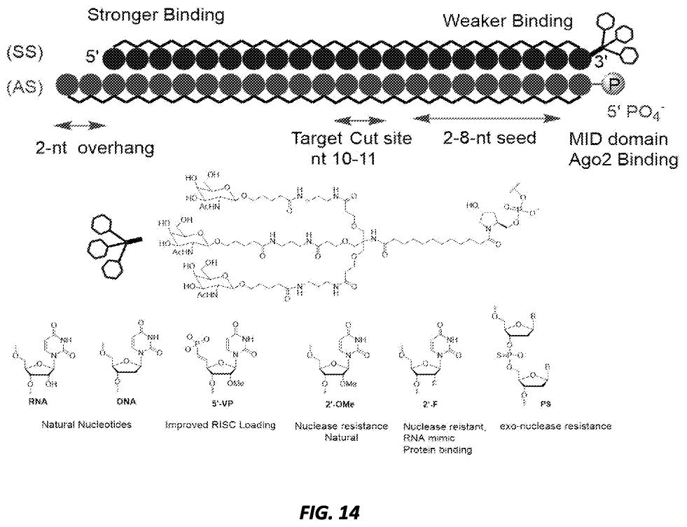

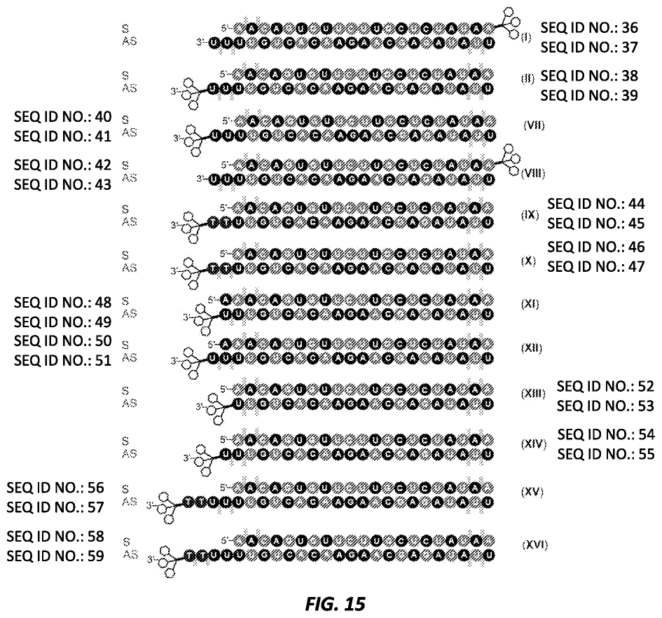

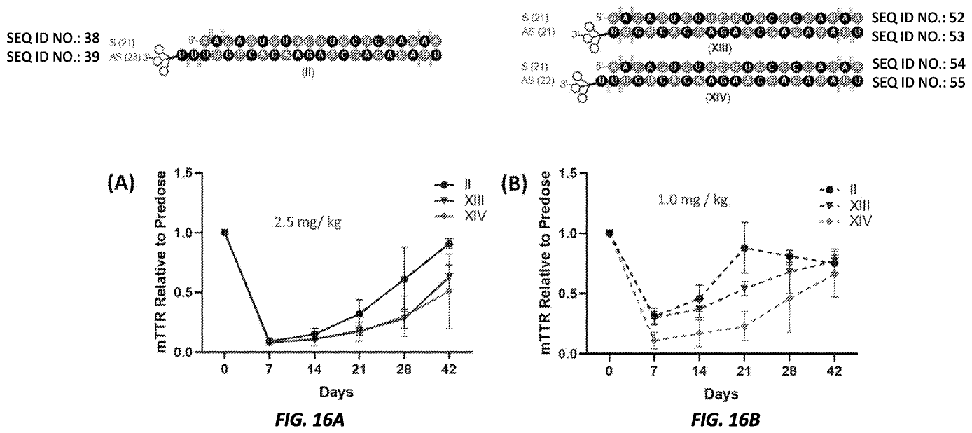

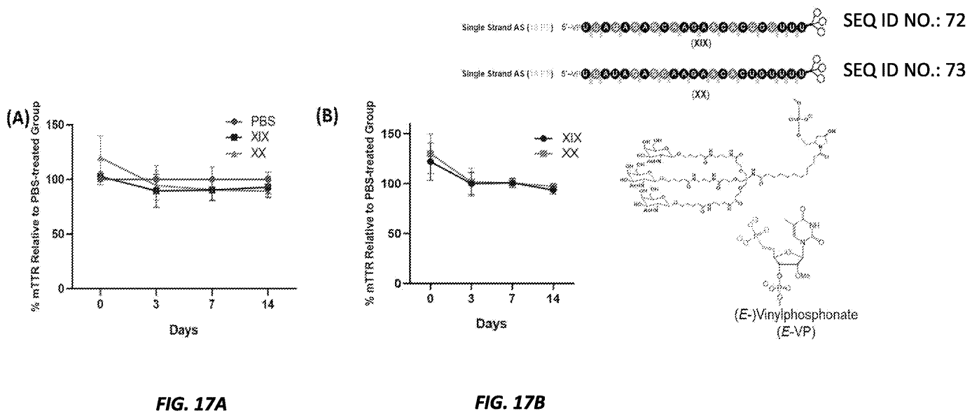

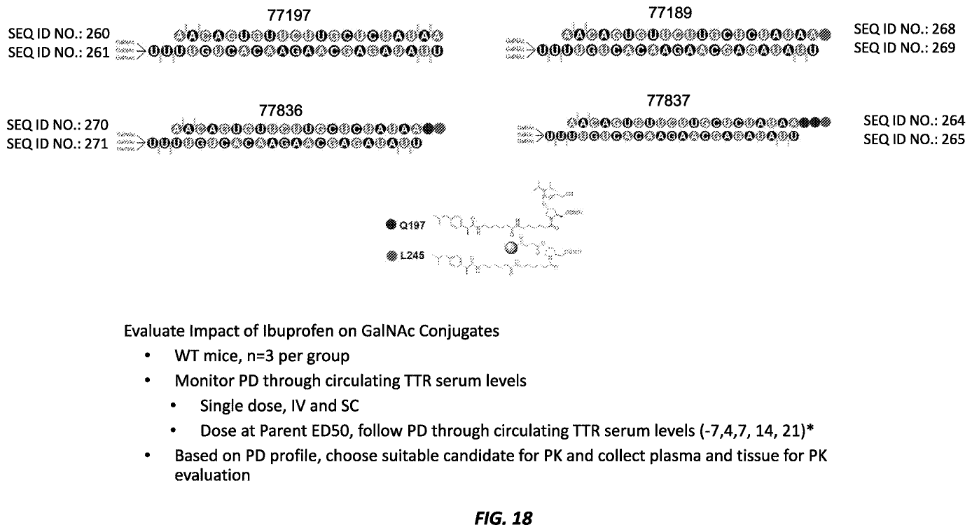

Activity of single-stranded antisense siRNAs comprising a 3′-conjugate for delivery and a 5′-vinylphosphonate nucleoside for facilitated loading to MID domain were tested. The results are shown in FIGS. 13A-13C and summarized in Table 7.

TABLE 7

| | | | | SEQ | | IC₅₀ [nm]^{e} | |
|---|---|---|---|---|---|---|---|
| | Sequence | S/AS | Sequence | ID | | Trans- | Free |
| Target^{[a]} | ID | (LN) | (5′-3′)^{[b]} | NO. | Kᵢ[nm]^{d} | fection | uptake |
| mTTR | XIX | AS | VPu•U•aU•aG•aG•cA•ag | 104 | n.d. | 120.62 | ND |
| | | (23) | •aA•cA•cU•gU•u•u•uL96 | | | | |
| Single | XX | AS) | VPu•U•au•aG•aG•C•aa•ga• | 105 | n.d. | 65.11 | ND |
| Sranded | | (23 | A•cA•cu•gu•u•u•uL96 | | | | |

^{[a]}siRNA targeting mRNA encoding mouse transthyretin (mTTR
^{[b]}S and AS indicate sense and antisense strands Italicized upper case and normal lower case letters indicate 2′-deoxy-2′-fluoro, and 2′-O-methyl sugar modifications, respectively, to adenosine (A), cytidine (C), guanosine (G) and uridine (U); and dT indicate deoxythymidine;
•Indicate phosphorothioate (PS) linkage.
^{d}Binding affinity to ASGPR (on mouse primary hepatocytes, fluorescent based assay).
^{e}Half maximal inhibitory concentration (IC₅₀) for in vitro gene silencing under transfection (Lipofectamine RNAiMAX) or free uptake conditions in primary mouse hepatocytes (LN) = length of nucleotide.

TABLE 8
m/r TTR targeting duplexes

| | | | SEQ | | SEQ |
|---|---|---|---|---|---|
| Du- | | | ID | Antisense | ID |
| plex | Id # | SensestrandoligoSeq | NO. | strandoligoSeq | NO. |
| I | AD- | AfsasCfaGfuGfuUfCfUfuGfcUf | 106 | usUfsaUfaGfaGfcAfagaAfcAfc | 124 |
| | 57727 | cUfaUfaAfL96 | | UfgUfususu | |
| II | AD- | AfsasCfaGfuGfuUfCfUfuGfcUf | 107 | usUfsaUfaGfaGfcAfagaAfcAfc | 125 |
| | 77197 | cUfaUfsasAf | | UfgUfususuL96 | |
| VII | AD- | AfsasCfaGfuGfuUfCfUfuGfcUf | 108 | usUfsaUfaGfaGfcAfagaAfcAfc | 126 |
| | 80301 | cUfaUfsasAf | | UfgUfuuuL96 | |
| VIII | AD- | AfsasCfaGfuGfuUfCfUfuGfcUf | 109 | usUfsaUfaGfaGfcAfagaAfcAfc | 127 |
| | 155481 | cUfaUfsasAfL96 | | UfgUfususu | |
| IX | AD- | AfsasCfaGfuGfuUfCfUfuGfcUf | 110 | usUfsaUfaGfaGfcAfagaAfcAfc | 128 |
| | 155482 | cUfaUfsasAf | | UfgUfudTdTL96 | |
| X | AD- | AfsasCfaGfuGfuUfCfUfuGfcUf | 111 | usUfsaUfaGfaGfcAfagaAfcAfc | 129 |
| | 155483 | cUfaUfsasAf | | UfgUfusdTsdTL96 | |
| XI | AD- | asAfsaCfaGfuGfuUfCfUfuGfcU | 112 | usUfsaUfaGfaGfcAfagaAfcAfc | 130 |
| | 157476 | fcUfaUfsasAf | | UfgUfsusuL96 | |
| XII | AD- | asAfsaCfaGfuGfuUfCfUfuGfcU | 113 | usUfsaUfaGfaGfcAfagaAfcAfc | 131 |
| | 157477 | fcUfaUfsasAf | | UfgUfususuL96 | |
| XIII | AD- | AfsasCfaGfuGfuUfCfUfuGfcUf | 114 | usUfsaUfaGfaGfcAfagaAfcAfc | 132 |
| | 192409 | cUfaUfsasAf | | UfgsUfsuL96 | |
| XIV | AD- | AfsasCfaGfuGfuUfCfUfuGfcUf | 115 | usUfsaUfaGfaGfcAfagaAfcAfc | 133 |
| | 192410 | cUfaUfsasAf | | UfgUfsusuL96 | |
| XV | AD- | AfsasCfaGfuGfuUfCfUfuGfcUf | 116 | usUfsaUfaGfaGfcAfagaAfcAfc | 134 |
| | 77196 | cUfaUfsasAf | | UfgUfususudTdTL96 | |
| XVI | AD- | AfsasCfaGfuGfuUfCfUfuGfcUf | 117 | usUfsaUfaGfaGfcAfagaAfcAfc | 135 |
| | 80302 | cUfaUfsasAf | | UfgUfuuusdTsdTL96 | |
| III | AD- | UfsgsAfcAfaAfaUfAfAfcUfcAf | 118 | usUfsaUfaGfuGfaGfuuaUfuUfu | 136 |
| | 58641 | cUfaUfaAfL96 | | GfuCfasasu | |
| IV | AD- | UfsgsAfcAfaAfaUfAfAfcUfcAf | 119 | usUfsaUfaGfuGfaGfuuaUfuUfu | 137 |
| | 81865 | cUfaUfsasAf | | GfuCfasasuL96 | |
| XVII | AD- | UfsgsAfcAfaAfaUfAfAfcUfcAf | 120 | usUfsaUfaGfuGfaGfuuaUfuUfu | 138 |
| | 81866 | cUfaUfsasAf | | GfuCfaauL96 | |
| VI | AD- | gsasaacuCfaAfUfAfaagugcuus | 121 | usAfsaagCfacuuuauUfgAfguuu | 139 |
| | 81867 | usa | | csusgL96 | |
| XVIII | AD- | gsasaacuCfaAfUfAfaagugcuus | 122 | usAfsaagCfacuuuauUfgAfguuu | 140 |
| | 81868 | usa | | cugL96 | |
| V | AD- | gsasaacuCfaAfUfAfaagugcuuu | 123 | usAfsaagCfacuuuauUfgAfguuu | 141 |
| | 74210 | aL96 | | csusg | |
| XIX | | (n/a, single stranded) | | VPusUfsaUfsaGfsaGfscAfsags | 142 |
| | | | | aAfscAfscUfsgUfsususuL96 | |
| XX | | (n/a, single stranded) | | VPusUfsausaGfsaGfsCfsaasga | 143 |
| | | | | sAfscAfscusgusususuL96 | |

### REFERENCES

1. Adams, D.; Gonzalez-Duarte, A.; O'Riordan, W. D.; Yang, C. C.; Ueda, M.; Kristen, A. V.; Tournev, I.; Schmidt, H. H.; Coelho, T.; Berk, J. L.; Lin, K. P.; Vita, G.; Attarian, S.; Plante-Bordeneuve, V.; Mezei, M. M.; Campistol, J. M.; Buades, J.; Brannagan, T. H., III; Kim, B. J.; Oh, J.; Parman, Y.; Sekijima, Y.; Hawkins, P. N.; Solomon, S. D.; Polydefkis, M.; Dyck, P. J.; Gandhi, P. J.; Goyal, S.; Chen, J.; Strahs, A. L.; Nochur, S. V.; Sweetser, M. T.; Garg, P. P.; Vaishnaw, A. K.; Gollob, J. A.; Suhr, O. B., Patisiran, an RNAi therapeutic, for hereditary transthyretin amyloidosis. N. Engl. J. Med. 2018, 379(1), 11-21.
2. Khvorova, A., Oligonucleotide Therapeutics—A New Class of Cholesterol-Lowering Drugs. New England Journal of Medicine 2017, 376(1), 4-7.
3. Ray, K. K.; Landmesser, U.; Leiter, L. A.; Kallend, D.; Dufour, R.; Karakas, M.; Hall, T.; Troquay, R. P. T.; Turner, T.; Visseren, F. L. J.; Wijngaard, P.; Wright, R. S.; Kastelein, J. J. P., Inclisiran in Patients at High Cardiovascular Risk with Elevated LDL Cholesterol. New England Journal of Medicine 2017, 376(15), 1430-1440.
4. Sheridan, C., PCSK9-gene-silencing, cholesterol-lowering drug impresses. Nature Biotechnology 2019, 37(12), 1385-1387.
5. Kosmas, C. E.; Estrella, A. M.; Sourlas, A.; Silverio, D.; Hilario, E.; Montan, P. D.; Guzman, E., Inclisiran: a new promising agent in the management of hypercholesterolemia. Diseases 2018, 6(3), 63.
6. Liebow, A.; Li, X.; Racie, T.; Hettinger, J.; Bettencourt, B. R.; Najafian, N.; Haslett, P.; Fitzgerald, K.; Holmes, R. P.; Erbe, D.; Querbes, W.; Knight, J., An Investigational RNAi Therapeutic Targeting Glycolate Oxidase Reduces Oxalate Production in Models of Primary Hyperoxaluria. Journal of the American Society of Nephrology 2017, 28(2), 494-503.
7. Chan, A.; Liebow, A.; Yasuda, M.; Gan, L.; Racie, T.; Maier, M.; Kuchimanchi, S.; Foster, D.; Milstein, S.; Charisse, K.; Sehgal, A.; Manoharan, M.; Meyers, R.; Fitzgerald, K.; Simon, A.; Desnick, R. J.; Querbes, W., Preclinical Development of a Subcutaneous ALAS1 RNAi Therapeutic for Treatment of Hepatic Porphyrias Using Circulating RNA Quantification. Mol. Ther.--Nucleic Acids 2015, 4(11), e263.
8. Severgnini, M.; Sherman, J.; Sehgal, A.; Jayaprakash, N. K.; Aubin, J.; Wang, G.; Zhang, L.; Peng, C. G.; Yucius, K.; Butler, J.; Fitzgerald, K., A rapid two-step method for isolation of functional primary mouse hepatocytes: cell characterization and asialoglycoprotein receptor based assay development. Cytotechnology 2012, 64(2), 187-95.
9. Nair, J. K.; Willoughby, J. L.; Chan, A.; Charisse, K.; Alam, M. R.; Wang, Q.; Hoekstra, M.; Kandasamy, P.; Kel'in, A. V.; Milstein, S.; Taneja, N.; O'Shea, J.; Shaikh, S.; Zhang, L.; van der Sluis, R. J.; Jung, M. E.; Akinc, A.; Hutabarat, R.; Kuchimanchi, S.; Fitzgerald, K.; Zimmermann, T.; van Berkel, T. J.; Maier, M. A.; Rajeev, K. G.; Manoharan, M., Multivalent N-acetylgalactosamine-conjugated siRNA localizes in hepatocytes and elicits robust RNAi-mediated gene silencing. J Am Chem Soc 2014, 136(49), 16958-61.
10. Elkayam, E.; Parmar, R.; Brown, C. R.; Willoughby, J. L.; Theile, C. S.; Manoharan, M.; Joshua-Tor, L., siRNA carrying an (E)-vinylphosphonate moiety at the 5′ end of the guide strand augments gene silencing by enhanced binding to human Argonaute-2. Nucleic Acids Research 2017, 45(6), 3528-3536.
11. Pei, Y.; Hancock, P. J.; Zhang, H.; Bartz, R.; Cherrin, C.; Innocent, N.; Pomerantz, C. J.; Seitzer, J.; Koser, M. L.; Abrams, M. T.; Xu, Y.; Kuklin, N. A.; Burke, P. A.; Sachs, A. B.; Sepp-Lorenzino, L.; Barnett, S. F., Quantitative evaluation of siRNA delivery in vivo. RNA 2010, 16(12), 2553-2563.
12. Chen, C.; Ridzon, D. A.; Broomer, A. J.; Zhou, Z.; Lee, D. H.; Nguyen, J. T.; Barbisin, M.; Xu, N. L.; Mahuvakar, V. R.; Andersen, M. R.; Lao, K. Q.; Livak, K. J.; Guegler, K. J., Real-time quantification of microRNAs by stem-loop RT-PCR. Nucleic Acids Research 2005, 33 (20), e179-e179.
13. Nair, J. K.; Attarwala, H.; Sehgal, A.; Wang, Q.; Aluri, K.; Zhang, X.; Gao, M.; Liu, J.; Indrakanti, R.; Schofield, S.; Kretschmer, P.; Brown, C. R.; Gupta, S.; Willoughby, J. L. S.; Boshar, J. A.; Jadhav, V.; Charisse, K.; Zimmermann, T.; Fitzgerald, K.; Manoharan, M.; Rajeev, K. G.; Akinc, A.; Hutabarat, R.; Maier, M. A., Impact of enhanced metabolic stability on pharmacokinetics and pharmacodynamics of GalNAc-siRNA conjugates. Nucleic Acids Research 2017, gkx818-gkx818.
14. Chan, A.; Liebow, A.; Yasuda, M.; Gan, L.; Racie, T.; Maier, M.; Kuchimanchi, S.; Foster, D.; Milstein, S.; Charisse, K.; Sehgal, A.; Manoharan, M.; Meyers, R.; Fitzgerald, K.; Simon, A.; Desnick, R. J.; Querbes, W., Preclinical Development of a Subcutaneous ALAS1 RNAi Therapeutic for Treatment of Hepatic Porphyrias Using Circulating RNA Quantification. Molecular Therapy-Nucleic Acids 2015, 4 (Supplement C), e263.
15. Cheng, A.; Li, M.; Liang, Y.; Wang, Y.; Wong, L.; Chen, C.; Vlassov, A. V.; Magdaleno, S., Stem-loop RT-PCR quantification of siRNAs in vitro and in vivo. Oligonucleotides 2009, 19 (2), 203-8.

### ADDITIONAL REFERENCES

1. Manoharan, M. (2004)RNA interference and chemically modified small interfering RNAs. Curr. Opin. in Chem.l Biol., 8, 570-579.
2. Shen, X. and Corey, D. R. (2017)Chemistry, mechanism and clinical status of antisense oligonucleotides and duplex RNAs. Nucleic Acids Res., 46, 1584-1600.
3. Setten, R. L., Rossi, J. J. and Han, S.-P. (2019) The current state and future directions of RNAi-based therapeutics. Nat. Rev. Drug Discov., 18, 421-446.
4. Bumcrot, D., Manoharan, M., Koteliansky, V. and Sah, D. W. (2006)RNAi therapeutics: a potential new class of pharmaceutical drugs. Nat. Chem. Biol., 2, 711-719.
5. T. C. Roberts, R. Langer and M. J. A. Wood, Nat. Rev. Drug. Discov., 2020, 19, 673. Deleavey, G. F., Watts, J. K. and Damha, M. J. (2009)Chemical modification of siRNA. Curr. Protoc. in Nucleic Acid Chem., 39, 16.13.11-16.13.22.
6. Akinc, A., Maier, M. A., Manoharan, M., Fitzgerald, K., Jayaraman, M., Barros, S., Ansell, S., Du, X., Hope, M. J., Madden, T. D., Mui, B. L., Semple, S. C., Tam, Y. K., Ciufolini, M., Witzigmann, D., Kulkarni, J. A., van der Meel, R. and Cullis, P. R. (2019) The Onpattro story and the clinical translation of nanomedicines containing nucleic acid-based drugs. Nat. Nanotechnol., 14, 1084-1087.
7. Balwani, M., Sardh, E., Ventura, P., Peiró, P. A., Rees, D. C., Stölzel, U., Bissell, D. M., Bonkovsky, H. L., Windyga, J., Anderson, K. E., Parker, C., Silver, S. M., Keel, S. B., Wang, J.-D., Stein, P. E., Harper, P., Vassiliou, D., Wang, B., Phillips, J., Ivanova, A., Langendonk, J. G., Ph.D., Kauppinen, R., Minder, E., Horie, Y., Penz, C., Chen, J., Liu, S., Ko, J. J., Sweetser, M. T., Garg, P., Vaishnaw, A., Kim, J. B., Simon, A. R. and Gouya, L. (2020) Phase 3 trial of RNAi therapeutic Givosiran for acute intermittent porphyria. N. Engl. J. Med., 382, 2289-2301.
8. Chan, A., Liebow, A., Yasuda, M., Gan, L., Racie, T., Maier, M., Kuchimanchi, S., Foster, D., Milstein, S., Charisse, K., Sehgal, A., Manoharan, M., Meyer, R., Fitzgerald, K., Simon, A., Desnick, R. J. and Querbes, W. (2015) Preclinical development of a subcutaneous ALAS1 RNAi therapeutic for treatment of hepatic porphyrias using circulating RNA quantification. Mol. Ther.-Nucleic Acids, 4, e263.
9. Liebow, A., Li, X., Racie, T., Hettinger, J., Bettencourt, B. R., Najafian, N., Haslett, P., Fitzgerald, K., Holmes, R. P., Erbe, D., Querbes, W. and Knight, J. (2017) An investigational RNAi therapeutic targeting glycolate oxidase reduces oxalate production in models of primary hyperoxaluria. J. Am. Soc. Nephrol., 28, 494-503.
10. Raal, F. J., Kallend, D., Ray, K. K., Turner, T., Koenig, W., Wright, R. S., Wijngaard, P. L. J., Curcio, D., Jaros, M. J., Leiter, L. A. and Kastelein, J. J. P. (2020) Inclisiran for the treatment of heterozygous familial hypercholesterolemia. N. Engl. J. Med., 382, 1520-1530.
11. Ray, K. K., Wright, R. S., Kallend, D., Koenig, W., Leiter, L. A., Raal, F. J., Bisch, J. A., Richardson, T., Jaros, M., Wijngaard, P. L. J. and Kastelein, J. J. P. (2020) Two phase 3 trials of inclisiran in patients with elevated LDL cholesterol. N. Engl. J. Med., 382, 1507-1519.
12. Fitzgerald, K., White, S., Borodovsky, A., Bettencourt, B. R., Strahs, A., Clausen, V., Wijngaard, P., Horton, J. D., Taubel, J., Brooks, A., Fernando, C., Kauffman, R. S., Kallend, D., Vaishnaw, A. and Simon, A. (2016) A highly durable RNAi therapeutic inhibitor of PCSK9. N. Engl. J. Med., 376, 41-51.
13. Allerson, C. R., Sioufi, N., Jarres, R., Prakash, T. P., Naik, N., Berdeja, A., Wanders, L., Griffey, R. H., Swayze, E. E. and Bhat, B. (2005) Fully 2′-modified oligonucleotide duplexes with improved in vitro potency and stability compared to unmodified small interfering RNA. J. Med. Chem., 48, 901-904.
14. Manoharan, M., Akinc, A., Pandey, R. K., Qin, J., Hadwiger, P., John, M., Mills, K., Charisse, K., Maier, M. A., Nechev, L., Greene, E. M., Pallan, P. S., Rozners, E., Rajeev, K. G. and Egli, M. (2011) Unique gene-silencing and structural properties of 2′-fluoro-modified siRNAs. Angew. Chem. Int. Ed. Engl., 50, 2284-2288.
15. Pallan, P. S., Greene, E. M., Jicman, P. A., Pandey, R. K., Manoharan, M., Rozners, E. and Egli, M. (2011) Unexpected origins of the enhanced pairing affinity of 2′-fluoro-modified RNA. Nucleic Acids Res., 39, 3482-3495.
16. Patra, A., Paolillo, M., Charisse, K., Manoharan, M., Rozners, E. and Egli, M. (2012)₂′-Fluoro RNA shows increased Watson-Crick H-bonding strength and stacking relative to RNA: evidence from NMR and thermodynamic data. Angew. Chem. Int. Ed. Engl., 51, 11863-11866.
17. Egli, M. and Manoharan, M. (2019)Re-engineering RNA molecules into therapeutic agents. Acc. Chem. Res., 52, 1036-1047.
18. Nair, J. K., Willoughby, J. L. S., Chan, A., Charisse, K., Alam, M. R., Wang, Q., Hoekstra, M., Kandasamy, P., Kel'in, A. V., Milstein, S., Taneja, N., O'Shea, J., Shaikh, S., Zhang, L., van der Sluis, R. J., Jung, M. E., Akinc, A., Hutabarat, R., Kuchimanchi, S., Fitzgerald, K., Zimmermann, T., van Berkel, T. J. C, Maier, M. A., Rajeev, K. G. and Manoharan, M. (2014) Multivalent N-acetylgalactosamine-conjugated siRNA localizes in hepatocytes and elicits robust RNAi-mediated gene silencing. J. Am. Chem. Soc., 136, 16958-16961.

### Example 2: Building Efficient RISC Blockers: Rational Optimization of the 5′ End of the siRNA Sense Strand to Ensure Strand Bias

To ensure specificity of small interfering RNAs (siRNAs), the antisense strand must be selected by the RNA induced silencing complex (RISC). To ensure strand bias, 5′-modified nucleotide building blocks were designed and synthesized to block interaction of RISC with the sense strand of the siRNA duplex. The modifications were designed based on the known structure of the MID domain of the enzyme Argonaute2. siRNAs with modified sense strands were evaluated in mice and in vitro. Our data show that the 5′-morpholino modification is an effective RISC blocker that will be useful for off-target mitigation.

Small interfering RNAs (siRNAs) are 21-23-nucleotide long duplexes that engage with the RNA-induced silencing complex (RISC) to regulate gene expression through the RNA interference (RNAi) pathway. RISC separates the antisense strand from the sense strand of the duplex and retains the antisense strand and then binds and cleaves target mRNA.¹⁻³ Strand selection is a critical step in siRNA-mediated gene silencing, as loading of the sense strand into the RISC can lead to off-target effects through silencing of mRNAs complementary to this strand.^{4,5} One driver of strand selection is thermodynamics: The strand with its 5′-terminus at the thermodynamically less stable end of the siRNA duplex is selected as the antisense strand.⁶ Moreover, 5′-end phosphorylation is a requirement for efficient loading into the RISC.^{7, 8, 9} Therefore, the presence of a monophosphate group or phosphate analog at the 5′ end can ensure selection of the desired strand.¹⁰⁻¹³ The presence of a group that blocks 5′-end phosphorylation of the sense strand also reduces off-target effects.¹⁸

We previously reported synthesis of a 5′-morpholino modified nucleoside (Mo1, FIG. 33) and demonstrated that its presence at the 5′-end of the sense strand improves antisense strand selection more effectively than 5′-O-methyl or unlocked nucleic acid.¹⁴ When the interaction of an siRNA with a Mol-modified strand with the MID domain of Ago2 was modeled, there was not a snug fit. We reasoned that extension of the morpholino group at the point of attachment to the nucleoside 5′-end might result in a better RISC antagonist. Thus, an extended morpholino, Mo2, was designed and synthesized (FIG. 33). We also synthesized two additional potential antagonists, piperidine (Pip) and Mo3 (FIG. 33) taking advantage of our recent demonstration that the 5′-OH of nucleosides can be bis-functionalized with aminooxy click chemistry.¹⁵

Mo2 was synthesized as shown in Scheme 1.

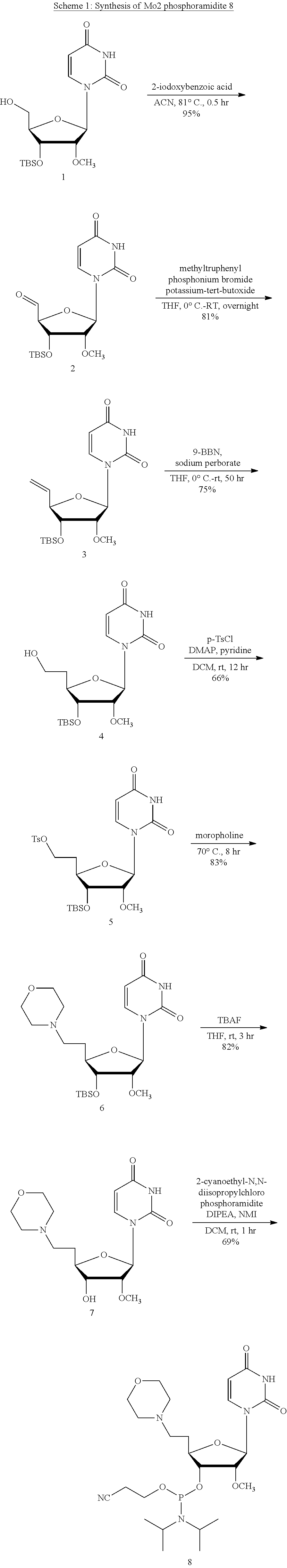

Commercially available nucleoside 1¹³ was oxidized to the aldehyde 2¹³ following the literature procedure.¹⁶ A Wittig reaction on compound 2 produced compound 3 in good yield. Hydroboration of 3 with 9-borabicyclo[3.3.1]nonane followed by oxidation afforded compound 4. This step was optimized after several trial reactions (Table 9). The primary hydroxyl group of 4 was then tosylated to obtain compound 5. Nucleophilic displacement of the tosyl group by neat morpholine under heating condition produced 6. Deprotection of the tert-butyldimethylsilyl (TBS) group using tetrabutylammonium fluoride (TBAF) afforded compound 7, which was then converted to phosphoramidite 8 by phosphitylation with 2-cyanoethyl-N,N-diisopropylchlorophosphoramidite in the presence of diisopropylethylamine (DIPEA) and N-methylimidazole (NMI).

TABLE 9
Optimization of conditions for compound 4

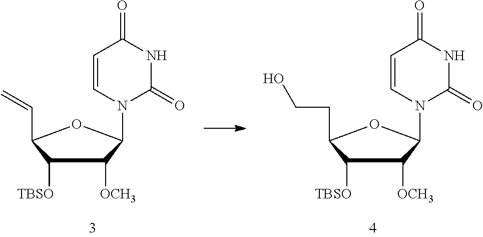

| Entry | Conditions^{a} | Product(s) (based on LCMS) |
|---|---|---|
| 1 | 9-BBN, sodium perborate tetrahydrate, THF, MeOH, H₂O, 0- | 4 (7%), |
| | 25° C., 17 hr | 3 (70%) |
| 2 | 9-BBN, sodium perborate tetrahydrate, THF, MeOH, H₂O, 0- | 4 (14%), |
| | 25° C., 17 hr | 3 (69%) |
| 3 | 9-BBN, sodium perborate tetrahydrate, THF, MeOH, H₂O, | 4 (32%), |
| | 30° C., 17 hr | 3 (21%) |
| 4 | BH₃.THF, sodium perborate tetrahydrate, THF, MeOH, H₂O, | 85% unidentified byproduct |
| | 0-25° C., 3 hr | |
| 5 | 9-BBN, sodium perborate tetrahydrate, THF, MeOH, H₂O, | 4 (43%) |
| | 40° C., 3 hr | |
| 6 | 9-BBN,sodium perborate tetrahydrate, THF, MeOH, H₂O, | 4 (75%) |
| | 0º C.-rt, 50 hr | |

^{a}Entry 5 is the optimized condition during large scale synthesis of 4

Pip and Mo3 building blocks were synthesized as shown in Scheme 2.

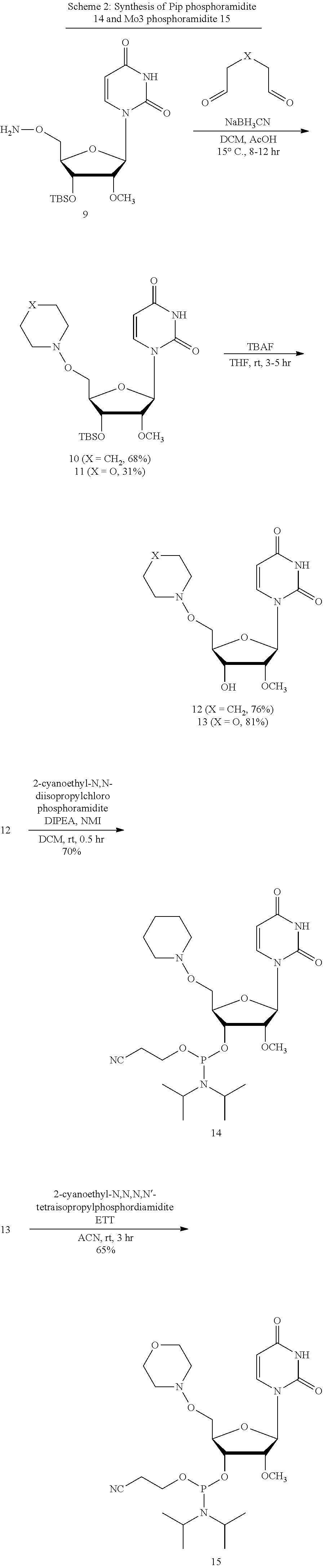

To synthesize the Pip and Mo3 building blocks (Scheme 2), compound 9 was synthesized following the recently reported procedure with an aminooxy (—ONH₂) group at the 5′-end of nucleoside.¹⁵ Reductive amination of 9 with glutaraldehyde resulted in compound 10 with a six-membered heterocyclic ring comprised of the aminooxy nitrogen atom. Removal of the TBS protecting group afforded compound 12. Phosphitylation of 12 with 2-cyanoethyl-N,N-diisopropylchlorophosphoramidite produced phosphoramidite 14. Similarly, reaction of 2-(2-oxoethoxy) acetaldehyde¹⁷ with compound 9 under reductive amination conditions produced 11, which, upon deprotection of the silyl group with TBAF, resulted in compound 13. Compound 13 was phosphityated with 2-cyanoethyl-N,N,N′,N′-tetraisopropylphosphordiamidite and 5-(ethylthio)-1H-tetrazole to afford the phosphoramidite 15 in moderate yield.

The modified morpholino building blocks 8, 14 and 15 were incorporated at the 5′-ends of oligonucleotides using standard oligonucleotide synthesis conditions. These building blocks were used to synthesize both sense and antisense strands of siRNAs targeting Apob (Table 11). In the parent siRNA, the 5′-terminal nucleotide is 2′-OMe-U. We first evaluated silencing of Apob expression in mice by siRNA with sense strands modified with Mol, Mo2, Pip and Mo3 (duplexes I, III, IV, and V respectively, Table 10). Mice were treated subcutaneously with 3 mg kg⁻¹ of siRNA, and circulating Apob protein was quantified using an ELISA assay. As previously observed,¹⁸ we found that siRNA activity was improved compared to the parent compound when the sense strand was conjugated with Mol (FIG. 34). The duplexes with sense strands modified with new morpholino modifications Mo2, Pip, and Mo3 had better activity than the siRNA with the Mo1 modification (FIG. 34).

TABLE 10
Exemplary siRNA duplexes

| | Sense strand (upper) and | SEQ |
|---|---|---|
| Du- | antisense strand | ID |
| plex | (lower)ª (5′-3′) | NO. |
| I | Mo1•g•UgAcAaAUAuGgGcAuCaAL | 144 |
| | u•U•gAuGcCcAuauUuGuCaCa•a•a | 145 |
| II | u•g•UgAcAaAUAuGgGcAuCaAL | 146 |
| | Mo1•U•gAuGcCcAuauUuGuCaCa•a•a | 147 |
| III | Mo2•g•UgAcAaAUAuGgGcAuCaAL | 148 |
| | u•U•gAuGcCcAuauUuGuCaCa•a•a | 149 |
| IV | Pip•g•UgAcAaAUAuGgGcAuCaAL | 150 |
| | u•U•gAuGcCcAuauUuGuCaCa•a•a | 151 |
| V | Mo3•g•UgAcAaAUAuGgGcAuCaAL | 152 |
| | u•U•gAuGcCcAuauUuGuCaCa•a•a | 153 |
| VI | u•g•UgAcAaAUAuGgGcAuCaAL | 154 |
| | Mo2•UsgAuGcCcAuauUuGuCaCa•a•a | 155 |
| VII | u•g•UgAcAaAUAuGgGcAuCaAL | 156 |
| | Pip•U•gAuGcCcAuauUuGuCaCa•a•a | 157 |
| VIII | u•g•UgAcAaAUAuGgGcAuCaAL | 158 |
| | Mo3•U•gAuGcCcAuauUuGuCaCa•a•a | 159 |

^{a}Chemical modifications are indicated as follows:
•, PS linkage; lower case, 2′-OMe; italicized upper case, 2′-F; L, trivalent-GalNAc respectively. Structures of Mo1, Mo2, Mo3, Pip and L are shown in FIG. 33.

| | |
|---|---|
| “Mo1•” | 5′-deoxy-5′-morpholino-2′-O-methyluridine-3′-phosphorothioate |
| | 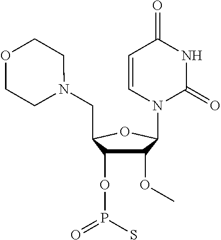 |
| “Mo2•” | 5′-deoxy-5′-(N-morpholinylmethyl)-2′-O- |
| | methyluridine-3′-phosphorothioate |
| | 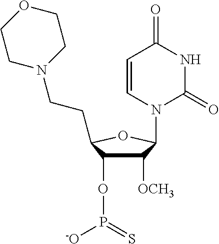 |
| “Mo3•” | 5′-O-(N-morpholinyl)-2′-O-methyluridine-3′-phosphorothioate |
| | 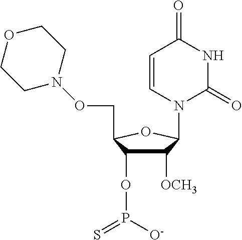 |
| “Pip•” | 5′-O-(N-piperidinyl)-2′-O-methyluridine-3′-phosphorothioate |
| | 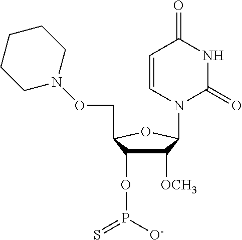 |
| “L” | L96 as described below. |

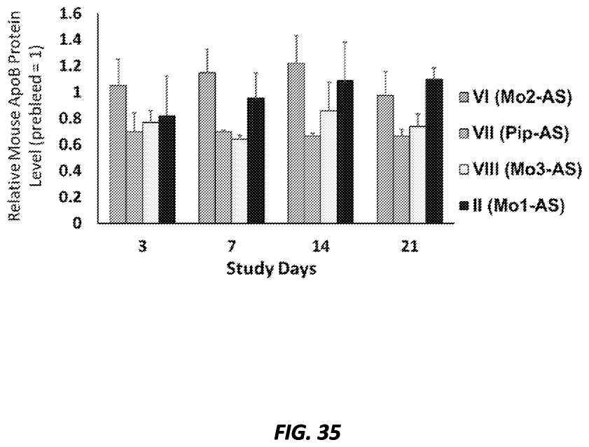

When placed at the 5′-end of the antisense strand (Table 12), all modifications resulted in loss of activity compared to the parent siRNA (FIG. 35). Consistent with the earlier observations,¹⁴ duplex II, modified with Mo1, was a poorer inhibitor of Apob silencing than the parent duplex. The siRNA modified with Mo2 was a poorer inhibitor than the duplex modified with Mol, and siRNAs modified with Pip and Mo3 were better inhibitors than were those modified with Mol or Mo2. These data are consistent with our hypothesis that these modifications all block 5′ phosphorylation, which is necessary for RISC loading.^{7, 8, 9}

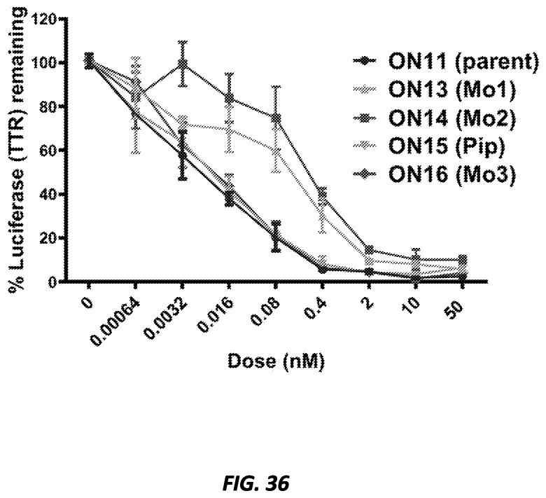

To evaluate the general utility of these modifications antisense strands of an siRNA targeting TTR were modified with the morpholino/piperidine analogues (Table 11) and the corresponding siRNAs were evaluated in a previously described in vitro luciferase reporter assay with a luciferase reporter plasmid that contains a single binding site for the antisense strand in the 3′ UTR.¹⁸ The siRNA with Mo1 on the antisense strand was 13-fold less potent than the parent strand; the Mo2 modification was superior, resulting in a 30-fold decrease in potency compared to parent (FIG. 36 and Table 13). Activities of siRNAs modified with Pip and Mo3 were similar to that of the parent siRNA (FIG. 36).

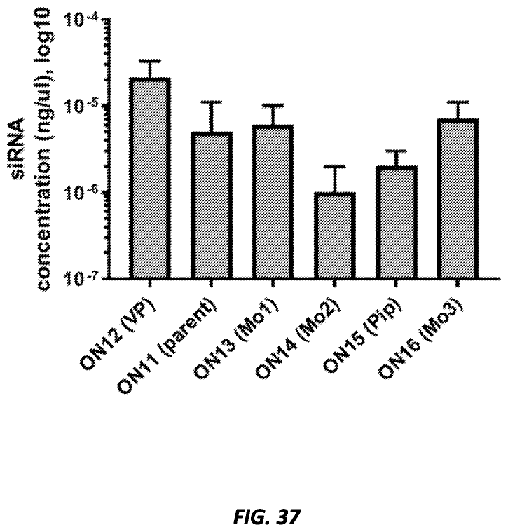

To assess the impact of the modification on the relative binding affinities to Ago2, the parent or morpholino-modified strands were incubated with recombinant human Ago2, and total RNA bound was quantified by stem-loop RT-qPCR. Significantly less oligonucleotide was loaded onto Ago2 when Mo2, Pip, or Mo3 were at the 5′ position of the antisense strand than when the antisense strand was not modified with a morpholino or when the Mo1 modification was present (FIG. 37).

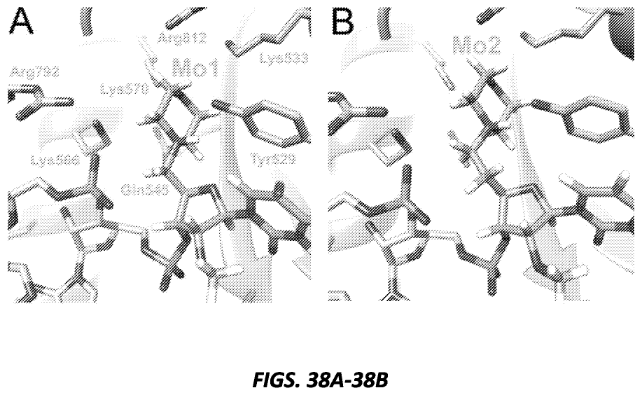

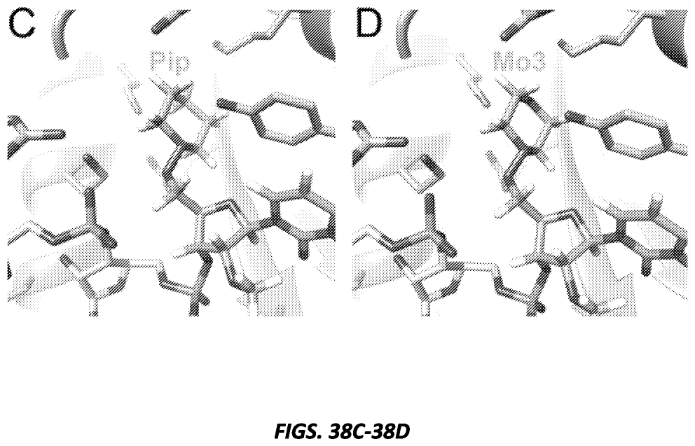

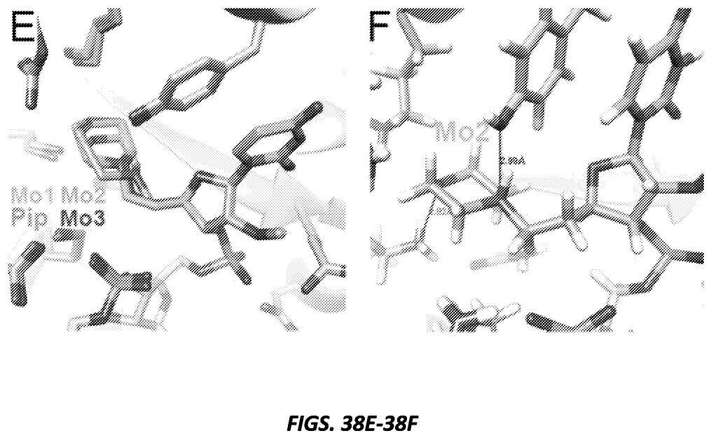

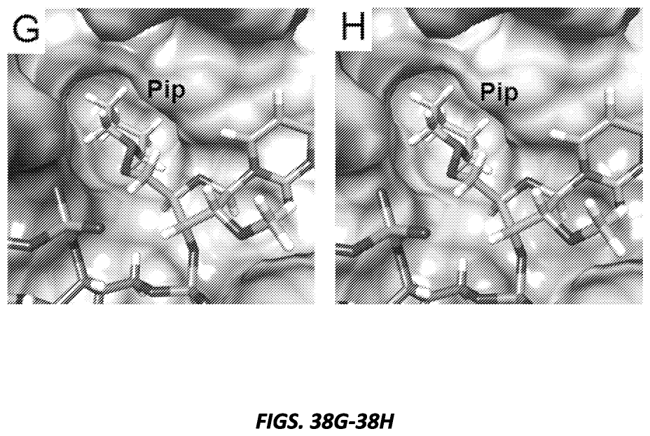

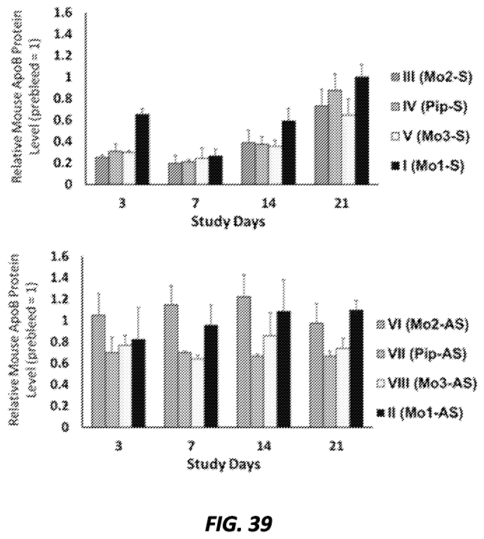

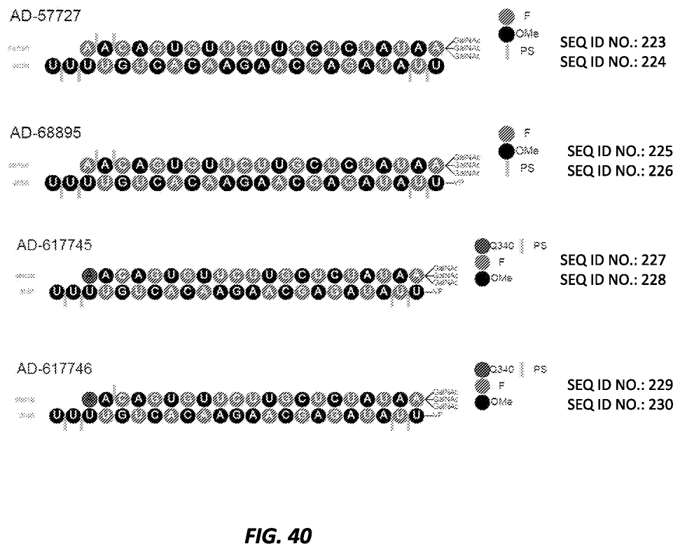

To rationalize our observations, we modeled complexes between Ago2 and antisense strands containing Mo1, Mo2, Pip, or Mo3 at their 5′-termini using the crystal structure of Ago2 bound to miR-20a (PDB ID 4f3t) as the starting structure.¹⁵ All models were built using UCSF Chimera¹⁹ and energy-minimized with Amber 14 (https://ambermd.org/),²⁰ as we did previously for modeling of the Mo1-modified strand bound to MID.²⁰ Multiple basic side chains are gathered around the 5′-phosphate of the antisense strand inside the Ago2 MID pocket, and interactions of Mo2, Pip, and Mo3 docked to MID are fairly similar to those seen with Mo1 (FIGS. 38A-38E). The Mo2 and Mo3 analogs, which are longer than Mo1, do not adopt a stretched orientation and thus are not inserted deeply into the binding pocket. These modifications are displaced by about 1 Å compared to the parent Mo1. In the energy minimizations, we assigned a +1 positive charge to Mo1 and Pip, but the corresponding nitrogen was neutral in Pip and Mo3. The only H-bond acceptors or donors on the morpholinos are a nitrogen lone pair or an N—H, respectively. The latter interaction is observed in the case of Mo2 with Tyr-529, which acts as an acceptor (FIG. 38F). The ring oxygen Pip, which is a rather weak acceptor, is within H bonding distance of Lys-570 (FIG. 38C). Interactions of the 5′ phosphate with all of the positively charged lysine and arginine residues as well Gln-545 are disrupted by the insertion of the 5′-morpholino modification. Compared to Mo1, the slightly longer Mo2, Pip, and Mo3 modifications have unfavorable steric interactions with these basic side chains. For example, Lys-533 has its NH₃⁺ headgroup turned away and presents a methylene in the direction of the morpholino moieties in complexes with strands with the longer modifications (FIGS. 38A-38E). All of the morpholino groups are electrostatically incompatible with the MID domain binding site (FIGS. 38G-38H). Mo2 incorporated at the 5′-end of the strand appears to be more disruptive than Mo1 as observed experimentally.

In conclusion, three phosphoramidite building blocks were synthesized to allow incorporation of extended morpholino functional groups at the 5′-position of oligonucleotides. In mice, in a reporter gene assay, and in an assay to monitor loading onto RISC, the Mo2 modification most effectively blocked loading of an siRNA strand of the modifications tested. This extended morpholino modification scan mitigate sense strand-mediated off-target effects²¹ and can be useful for studies of the role of the antisense strand in downstream effects.²² These modifications can also improve resistance to degradation by 5′-exonucleases. When used in sense strands in conjunction with the 5′-phosphate mimic 5′-vinylphosphonate, the Mo2 modification can enhance potency and specificity through multiple mechanisms.

### REFERENCES

(1) S. M. Elbashir, J. Harborth, W. Lendeckel, A. Yalcin, K. Weber and T. Tuschl, Nature, 2001, 411, 494-498.
(2) M. Manoharan, Curr. Opin. Chem. Biol., 2004, 8, 570-579.
(3) D. Bumcrot, M. Manoharan, V. Koteliansky and D. W. Y. Sah, Nat. Chem. Biol., 2006, 2, 711-719.
(4) N. M. Snead, J. R. Escamilla-Powers, J. J. Rossi and A. P. McCaffrey, Mol. Ther.--Nucleic Acids, 2013, 2, E103.
(5) N. Vaish, F. Chen, S. Seth, K. Fosnaugh, Y. Liu, R. Adami, T. Brown, Y. Chen, P. Harvie, R. Johns, G. Severson, B. Granger, P. Charmley, M. Houston, M. V. Templin and B. Polisky, Nucleic Acids Res., 2011, 39, 1823-1832.
(6) D. S. Schwarz, G. Hutvagner, T. Du, Z. Xu, N. Aronin and P. D. Zamore, Cell, 2003, 115, 199-208.
(7) S. Weitzer and J. Martinez, Nature, 2007, 447, 222-226.
(8) C.-D. Kuhn and L. Joshua-Tor, Trends Biochem. Sci., 2013, 38, 263-271.
(9) N. H. Tolia and L. Joshua-Tor, Nature Chem.l Biol., 2007, 3, 36-43.
(10) E. Elkayam, C.-D. Kuhn, A. Tocilj, A. D. Haase, E. M. Greene, G. J. Hannon and L. Joshua-Tor, Cell, 2012, 150, 100-110.
(11) N. T. Schirle and I. J. MacRae, Science, 2012, 336, 1037-1040.
(12) E. Elkayam, R. Parmar, C. R. Brown, J. L. Willoughby, C. S. Theile, M. Manoharan and L. Joshua-Tor, Nucleic Acids Res., 2017, 45, 3528-3536.
(13) R. G. Parmar, C. R. Brown, S. Matsuda, J. L. S. Willoughby, C. S. Theile, K. Charisse, D. J. Foster, I. Zlatev, V. Jadhav, M. A. Maier, M. Egli, M. Manoharan and K. G. Rajeev, J. Med. Chem., 2018, 61, 734-744.
(14) P. Kumar, R. G. Parmar, C. R. Brown, J. L. S. Willoughby, D. J. Foster, I. R. Babu, S. Schofield, V. Jadhav, K. Charisse, J. K. Nair, K. G. Rajeev, M. A. Maier, M. Egli and M. Manoharan, Chem. Commun., 2019, 55, 5139-5142.
(15) D. Datta, S. Mori, M. Madaoui, K. Wassarman, I. Zlatev and M. Manoharan, Org. Lett., 2022, 24, 4496-4501.
(16) M. J. Fer, P. Doan, T. Prange, S. Calvet-Vitale and C. Gravier-Pelletier, J. Org. Chem., 2014, 79, 7758-7765.
(17) M. Israel and R. J. Murray, J. Med. Chem., 1982, 25, 24-28.
(18) D. C. Guenther, S. Mori, S. Matsuda, J. A. Gilbert, S. Hyde, A. Bisbe, Y. Jiang, S. Agarwal, M. M. Janas, K. Charisse and M. Manoharan, J. Am. Chem. Soc., 2022.
(19) E. F. Pettersen, T. D. Goddard, C. C. Huang, G. S. Couch, D. M. Greenblatt, E. C. Meng and T. E. Ferrin, J. Comput. Chem., 2004, 25, 1605-1612.
(20) D. A. Case, T. E. Cheatham Iii, T. Darden, H. Gohlke, R. Luo, K. M. Merz Jr, A. Onufriev, C. Simmerling, B. Wang and R. J. Woods, J. Comput. Chem., 2005, 26, 1668-1688.
(21) V. Kotikam and E. Rozners, Acc. Chem. Res., 2020, 53, 1782-1790.
(22) M. M. Janas, M. K. Schlegel, C. E. Harbison, V. O. Yilmaz, Y. Jiang, R. Parmar, I. Zlatev, A. Castoreno, H. Xu, S. Shulga-Morskaya, K. G. Rajeev, M. Manoharan, N. D. Keirstead, M. A. Maier and V. Jadhav, Nat. Commun., 2018, 9, 1-10.

### Synthesis of Building Blocks:

General conditions: Compounds were visualized under UV light (254 nm) or after spraying with the p-anisaldehyde staining solution followed by heating. Flash column chromatography was performed using a Teledyne ISCO Combi Flash system with pre-packed RediSep Teledyne ISCO silica gel cartridges and Prep-Achiral supercritical fluid chromatography (SFC). All moisture-sensitive reactions were carried out under anhydrous conditions using dry glassware, anhydrous solvents, and argon atmosphere. All commercially available reagents and solvents were purchased from Sigma-Aldrich unless otherwise stated and were used as received. ESI-MS spectra were recorded on a Waters QTof Premier instrument using the direct flow injection mode. ¹H NMR spectra were recorded at 300, 400 and 500 MHz. ¹³C NMR spectra were recorded at 75, 101, and 126 MHz. ³¹P NMR spectra were recorded at 162 and 202 MHz. Chemical shifts are given in ppm referenced to the solvent residual peak (DMSO-d₆-¹H: δ at 2.50 ppm and ¹³C δ at 39.5 ppm; CDCl₃-¹H: δ at 7.26 ppm and ¹³C δ at 77.16 ppm). Coupling constants are given in Hertz. Signal splitting patterns are described as singlet(s), doublet (d), triplet (t), septet (sept), broad signal (brs), or multiplet (m)

### Synthesis of (2S,5R)-3-[tert-butyl(dimethyl) silyl]oxy-5-(2,4-dioxopyrimidin-1-yl)-4-methoxy-tetrahydrofuran-2-carbaldehyde (2)

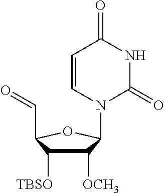

The aldehyde was synthesized following the literature procedure¹. 2-iodoxybenzoic acid (2.82 g, 10.07 mmol) was added to 1 (1.25 g, 3.36 mmol) in anhydrous acetonitrile (30 mL) under argon atmosphere. The mixture was refluxed at 81° C. for 0.75 hr and then cooled. Reaction mixture was filtered through celite bed and solid residue was washed with ethyl acetate (EtOAc) (50 mL). The combined filtrate was evaporated at 30° C. Gummy residue thus obtained, was further co-evaporated with toluene (30 mL) to afford 2 (1.15 g, 93% yield) as an amorphous white solid that was used in the next step without of further purification. The product was stored at −20° C. ¹H NMR (500 MHz, CDCl₃) δ 9.79 (s, 1H), 9.75 (s, 1H), 7.68 (d, J=8.1 Hz, 1H), 5.88-5.72 (m, 2H), 4.55 (d, J=4.5 Hz, 1H), 4.43 (t, J=4.5 Hz, 1H), 3.96 (t, J=4.7 Hz, 1H), 3.47 (s, 3H), 0.93 (s, 9H), 0.14 (d, J=7.7 Hz, 6H) ppm.

### Synthesis of 1-[(2R,5R)-4-[tert-butyl(dimethyl) silyl]oxy-3-methoxy-5-vinyl-tetrahydrofuran-2-yl]pyrimidine-2,4-dione (3)

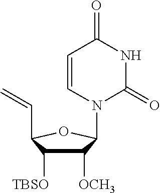

Compound 3 was obtained following the literature procedure¹. To a well-stirred suspension of methyltriphenylphosphonium bromide (7.08 g, 19.43 mmol) in tetrahydrofuran (THF) (30 mL) was added potassium-tert-butoxide (2.23 g, 19.43 mmol). The bright yellow suspension was stirred at 0° C. for 10 minutes and then for 1 hr. The crude aldehyde 2 (2.4 g, 6.48 mmol) was dissolved in THF (20 mL), transferred into a dropping funnel, and slowly added to the solution of ylide at 0° C. The mixture was vigorously stirred at 0° C. for 10 minutes and the at 22° C. for 16 hr. The mixture was diluted with DCM (30 mL) and organic layer was washed with saturated NH₄Cl solution (30 mL). Organic layer then separated, dried over anhydrous Na₂SO₄, filtered and the filtrate was evaporated to dryness. Crude compound was purified by column chromatography (gradient: 0-50% EtOAc in hexane) to afford 3 (1.93 g, 81% yield) as white foam. ¹H NMR (400 MHz, CDCl₃) δ 9.26 (s, 1H), 7.38 (d, J=8.1 Hz, 1H), 5.90 (ddd, J=17.1, 10.5, 6.5 Hz, 1H), 5.83 (d, J=2.0 Hz, 1H), 5.77 (dd, J=8.1, 1.9 Hz, 1H), 5.45 (dt, J=17.1, 1.3 Hz, 1H), 5.35 (dt, J=10.5, 1.3 Hz, 1H), 4.42 (tt, J=6.5, 1.3 Hz, 1H), 3.91 (dd, J=7.7, 5.0 Hz, 1H), 3.72 (dd, J=5.0, 2.1 Hz, 1H), 3.56 (s, 3H), 0.90 (s, 9H), 0.09 (d, J=7.2 Hz, 6H) ppm. ¹³C (101 MHz, CDCl₃) δ 163.4, 150.0, 139.8, 134.6, 119.3, 102.6, 89.8, 8.1, 83.6, 74.6, 58.8, 25.8, 18.3, −4.5, −4.5 ppm. HRMS calc. for C₁₇H₂₉N₂O₅Si [M+H]⁺ 369.1846, found 369.1846.

### Synthesis of 1-[(2R,5R)-4-[tert-butyl(dimethyl) silyl]oxy-5-(2-hydroxyethyl)-3-methoxy-tetrahydrofuran-2-yl]pyrimidine-2,4-dione (4)

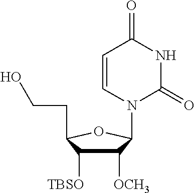

Hydroboration of 3 was done following the literature procedure². To a solution of 3 (2.0 g, 5.43 mmol) in THF (25 mL) was added 9-borabicyclo[3.3.1]nonane (3.97 g, 32.56 mmol, 4.44 mL) at 0° C. The mixture was allowed to warm and stirred at 22° C. for 20 hr. Then the reaction mixture was cooled, and methanol (MeOH) (20 mL) was added dropwise. When the gas evolution ceased, water (30 mL) was added followed by sodium perborate tetrahydrate (20.88 g, 130.26 mmol). Resulting mixture was stirred for 30 hr vigorously at 0° C. and then filtered. Filtrate was washed with EtOAc (50 mL). Organic layer was further washed with brine (40 mL), dried over anhydrous Na₂SO₄, filtered and filtrate was evaporated to dryness. Crude residue thus obtained was purified by column chromatography (gradient: 20-75% EtOAc in hexane) to afford 4 (1.57 g, 75% yield) as white solid. ¹H NMR (600 MHz, DMSO-d₆) δ 11.38 (s, 1H), 7.62 (d, J=8.0 Hz, 1H), 5.77 (d, J=4.5 Hz, 1H), 5.66 (d, J=8.0 Hz, 1H), 4.56 (t, J=5.0 Hz, 1H), 4.12 (t, J=5.2 Hz, 1H), 3.88 (dt, J=7.0, 4.4 Hz, 2H), 3.56-3.42 (m, 2H), 3.32 (s, 3H), 1.80 (dtd, J=14.3, 7.4, 4.6 Hz, 1H), 1.71 (ddt, J=14.0, 8.0, 5.5 Hz, 1H), 0.88 (s, 9H), 0.09 (d, J=3.6 Hz, 6H) ppm. ¹³C NMR (151 MHz, DMSO-d₆) δ 163.1, 150.4, 141.0, 102.2, 87.1, 81.5, 80.7, 73.4, 57.5, 57.4, 35.9, 25.7, 17.8, −4.7, −4.9 ppm. HRMS calc. for C₁₇H₃₀N₂O₆SiNa [M+Na]⁺ 409.1771, found 409.1767.

### Synthesis of 2-[(2R,5R)-3-[tert-butyl(dimethyl) silyl]oxy-5-(2,4-dioxopyrimidin-1-yl)-4-methoxy-tetrahydrofuran-2-yl]ethyl-4-methylbenzenesulfonate (5)

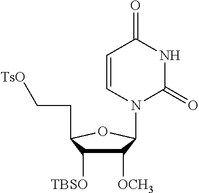

To a clear solution of 4 (1.00 g, 2.59 mmol) in dry dichloromethane (DCM) (30 mL) was added 4-(dimethylamino)pyridine (638.55 mg, 5.17 mmol) and reaction mixture was cooled to 0° C. To the resulting solution, p-toluenesulfonyl chloride (747.36 mg, 3.88 mmol) was added in single portion and reaction mixture was stirred for 12 hr at 22° C. Reaction mixture was diluted with DCM (20 mL), washed with NaHCO₃ solution (30 mL) and organic layer was separated. DCM layer was dried over anhydrous Na₂SO₄, filtered and filtrated was evaporated to dryness. The crude mass thus obtained, was purified by column chromatography (gradient: 0-60% EtOAc in hexane) to afford 5 (0.92 g, 66% yield) as white solid. ¹H NMR (600 MHz, CDCl₃) δ 8.61 (s, 1H), 7.81-7.76 (m, 2H), 7.37-7.32 (m, 2H), 7.23 (d, J=8.1 Hz, 1H), 5.76 (dd, J=8.1, 1.8 Hz, 1H), 5.64 (d, J=2.6 Hz, 1H), 4.23 (ddd, J=10.1, 7.1, 5.3 Hz, 1H), 4.13 (ddd, J=10.1, 7.7, 6.4 Hz, 1H), 3.94 (ddd, J=9.4, 7.3, 3.3 Hz, 1H), 3.86 (dd, J=7.3, 5.3 Hz, 1H), 3.73 (dd, J=5.3, 2.6 Hz, 1H), 3.48 (s, 3H), 2.45 (s, 3H), 2.14 (dtd, J=14.6, 7.4, 3.4 Hz, 1H), 1.90 (dddd, J=14.5, 9.4, 6.5, 5.4 Hz, 1H), 0.89 (s, 9H), 0.09 (s, 3H), 0.07 (s, 3H) ppm. ¹³C NMR (151 MHz, CDCl₃) δ 162.9, 149.7, 145.1, 140.3, 132.9, 130.0, 128.1, 102.8, 90.4, 83.0, 79.5, 74.4, 67.0, 58.6, 32.5, 25.8, 21.8, 18.2, −4.4, −4.7 ppm. HRMS calc. for C₂₄H₃₇N₂O₈SSi [M+H]⁺ 541.2040, found 541.2045.

### Synthesis of 1-[(2R,5R)-4-[tert-butyl(dimethyl) silyl]oxy-3-methoxy-5-(2-morpholinoethyl)tetrahydrofuran-2-yl]pyrimidine-2,4-dione (6)

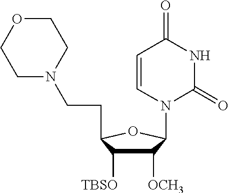

Morpholine (4 mL) was added to 5 (0.5 g, 0.924 mmol) and the clear solution was heated at 70° C. for 8 hr. All the volatile matters were evaporated, and the residue was purified by column chromatography (gradient: 0-5% MeOH in DCM) to afford 6 (0.35 g, 83% yield) as hygroscopic solid. ¹H NMR (500 MHz, CDCl₃) δ 9.05 (s, 1H), 7.33 (d, J=8.1 Hz, 1H), 5.78 (d, J=2.4 Hz, 1H), 5.76 (d, J=8.1 Hz, 1H), 4.03 (ddd, J=9.0, 7.4, 3.8 Hz, 1H), 3.84 (dd, J=7.4, 5.2 Hz, 1H), 3.75-3.63 (m, 5H), 3.52 (s, 3H), 2.67-2.34 (m, 6H), 2.05-1.88 (m, 1H), 1.79-1.64 (m, 1H), 0.91 (s, 9H), 0.10 (d, J=5.0 Hz, 6H) ppm. ¹³C NMR (126 MHz, CDCl₃) δ 163.2, 149.9, 139.9, 102.6, 89.6, 83.6, 81.5, 74.7, 67.0, 58.5, 55.4, 53.8, 30.4, 25.8, 18.3, −4.3, −4.6 ppm. HRMS calc. for C₂₁H₃₈N₃O₆Si [M+H]⁺456.2530, found 456.2529.

### Synthesis of 1-[(2R,5R)-4-hydroxy-3-methoxy-5-(2-morpholinoethyl)tetrahydrofuran-2-yl]pyrimidine-2,4-dione (7)

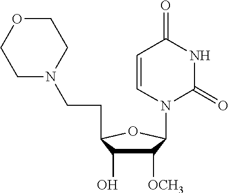

To a clear solution of 6 (0.6 g, 1.32 mmol) in THF (15 mL) at 22° C., tetrabutylammonium fluoride, 1M in THF (1.71 mmol, 1.71 mL) was added slowly in single portion and then stirred for 3 hrs. All the volatile matters were removed under high vacuum pump and the residue thus obtained was purified by column chromatography (gradient: 0-10% MeOH in DCM) to afford 7 (0.37 g, 82% yield) as white solid. ¹H NMR (500 MHz, DMSO-d₆) δ 11.50-11.08 (m, 1H), 7.59 (d, J=8.1 Hz, 1H), 5.77 (d, J=4.3 Hz, 1H), 5.65 (dd, J=8.1, 1.9 Hz, 1H), 5.37-5.18 (m, 1H), 3.90 (s, 1H), 3.84 (dd, J=5.3, 4.3 Hz, 1H), 3.79 (dt, J=8.0, 5.4 Hz, 1H), 3.57 (t, J=4.7 Hz, 4H), 3.36 (s, 3H), 2.40-2.26 (m, 6H), 1.87 (dtd, J=13.1, 7.8, 5.2 Hz, 1H), 1.71 (dtd, J=13.5, 7.7, 5.5 Hz, 1H) ppm. ¹³C NMR (126 MHz, DMSO-d₆) δ 163.0, 150.4, 140.8, 102.1, 87.0, 82.0, 81.7, 72.1, 66.1, 57.6, 54.6, 53.2, 29.7 ppm. HRMS calc. for C₁₅H₂₄N₃O₆ [M+H]⁺ 342.1665, found 342.1660.

### Synthesis of 3-[(diisopropylamino)-[(2R,5R)-5-(2,4-dioxopyrimidin-1-yl)-4-methoxy-2-(2-morpholinoethyl)tetrahydrofuran-3-yl]oxy-phosphanyl]propanenitrile (8)

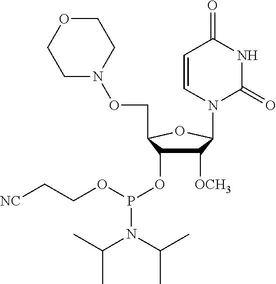

To a clear solution of 7 (0.34 g, 1.0 mmol) in DCM (20 mL) was added DIPEA (650.13 mg, 4.98 mmol, 0.88 mL) and N-methylimidazole (123.90 mg, 1.49 mmol, 0.12 mL) in single portions. To the resulting mixture was added 2-cyanoethyl-N,N-diisopropylchlorophosphoramidite (248.15 mg, 996.02 μmol, 0.23 m μL) at 22° C. and stirred for 1 hr. After 1 hr when TLC showed completion of reaction and reaction mixture was diluted with DCM (20 mL) and quenched by adding NaHCO₃ solution (20 mL). Organic layer was separated, dried over anhydrous Na₂SO₄, filtered and filtrate was evaporated to dryness. Crude material was triturated with 1:1 hexane in ether. Precipitate thus obtained was purified by column chromatography (gradient: 0-3% MeOH in DCM containing 3% TEA) to afford 8 (0.36 g, 69% yield) as yellowish white hygroscopic foam. ¹H NMR (400 MHz, CD₃CN) δ 8.84 (s, 1H), 7.40 (dd, J=8.1, 1.0 Hz, 1H), 7.14-6.73 (m, 1H), 5.83 (dd, J=4.5, 1.1 Hz, 1H), 5.63 (d, J=8.1 Hz, 1H), 4.29-3.97 (m, 2H), 3.91-3.75 (m, 2H), 3.69-3.55 (m, 6H), 3.43 (d, J=14.2 Hz, 3H), 2.83-2.60 (m, 2H), 2.49-2.26 (m, 4H), 1.94 (dt, J=4.9, 2.5 Hz, 1H), 1.76 (ddd, J=9.9, 6.3, 2.2 Hz, 1H), 1.29-0.97 (m, 13H) ppm. ¹³C NMR (126 MHz, CD₃CN) δ 164.1, 151.5, 151.5, 141.1, 138.9, 129.5, 121.3, 119.6, 103.2, 103.1, 88.9, 88.5, 83.2, 83.2, 82.8, 82.7, 82.4, 82.3, 82.0, 81.9, 75.3, 75.2, 75.1, 74.9, 74.5, 67.5, 67.5, 59.8, 59.6, 59.2, 59.0, 58.9, 58.8, 58.7, 58.7, 58.0, 55.6, 54.6, 54.6, 47.3, 46.6, 46.4, 46.3, 46.0, 46.0, 45.6, 44.2, 44.1, 44.1, 44.0, 33.6, 30.9, 25.0, 24.9, 24.9, 24.9, 23.6, 23.2, 23.2, 23.1, 23.1, 22.6, 21.1, 21.0, 21.0, 20.4, 20.4 ppm. ³¹P NMR (202 MHz, CD₃CN) δ 150.84, 150.78 ppm. HRMS calc. for C₂₄H₄₁N₅O₇P [M+H]⁺ 542.2744, found 542.2744.

### Synthesis of 1-[(2R,5R)-4-[tert-butyl(dimethyl) silyl]oxy-3-methoxy-5-(1-piperidyloxymethyl)tetrahydrofuran-2-yl]pyrimidine-2,4-dione (10)

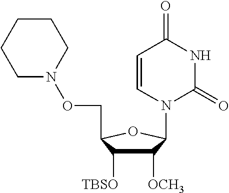

To a clear solution of 9 (0.4 g, 1.03 mmol) in acetic acid (5 mL) and DCM (10 mL), was added glutaraldehyde (0.1 g, 1.03 mmol). To the resulting mixture, sodium cyanoborohydride (0.74 g, 11.56 mmol) was added in portions at 15° C. The reaction mixture was further diluted with DCM (70 mL) and stirred for 8 hr. Volatile matters were removed under high vacuum and the residue thus obtained, was diluted with DCM (50 mL), and washed with water (3×30 mL). Organic layer was separated, dried over anhydrous Na₂SO₄, filtered and filtrate was evaporated to dryness. The crude compound thus obtained was purified by column chromatography to afford 10 (0.32 g, 68.0% yield). ¹H NMR (400 MHz, CDCl₃) δ 9.18 (s, 1H), 8.07 (d, J=8.2 Hz, 1H), 5.88 (d, J=1.9 Hz, 1H), 5.69 (dd, J=8.1, 2.1 Hz, 1H), 4.36-3.96 (m, 3H), 3.97-3.72 (m, 1H), 3.61 (dd, J=4.7, 1.9 Hz, 1H), 3.54 (s, 3H), 3.41-3.24 (m, 2H), 2.36 (s, 2H), 1.74 (s, 2H), 1.56 (d, J=18.3 Hz, 3H), 1.24-1.11 (m, 1H), 0.90 (s, 9H), 0.09 (d, J=3.4 Hz, 6H) ppm. ¹³C NMR (126 MHz, CDCl₃) δ 163.5, 150.2, 140.5, 101.7, 88.1, 84.2, 82.5, 69.4, 68.7, 58.5, 56.9, 25.8, 25.5, 23.5, 18.3, −4.5, −4.7 ppm. HRMS calc. for C₂₁H₃₈N₃O6Si [M+H]⁺ 456.2530, found 456.2520.

### Synthesis of 1-[(2R,5R)-4-[tert-butyl(dimethyl) silyl]oxy-3-methoxy-5-(morpholinooxymethyl)tetrahydrofuran-2-yl]pyrimidine-2,4-dione (11)

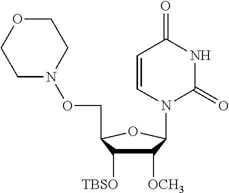

To a clear solution of 9 (2.4 g, 6.19 mmol) in acetic acid (20 mL) was added 2-(2-oxoethoxy) acetaldehyde³ (0.63 g, 6.19 mmol) in followed by sodium cyanoborohydride (4.13 g, 64.4 mmol) in portions and stirred at 15° C. for 12 hr. Diluted the mixture with DCM (50 mL) and organic layer was washed with water (2×30 mL). DCM layer dried over anhydrous Na₂SO₄, filtered and the filtrate was evaporated to dryness. The crude residue was purified by column chromatography to afford 11 (0.88 g, 31% yield) as white solid. ¹H NMR (500 MHz, CDCl₃) δ 8.50 (s, 1H), 7.93 (d, J=8.2 Hz, 1H), 5.86 (d, J=2.0 Hz, 1H), 5.70 (dd, J=8.2, 2.0 Hz, 1H), 4.23-4.06 (m, 3H), 3.92 (dd, J=11.9, 3.0 Hz, 3H), 3.67-3.57 (m, 3H), 3.55 (s, 3H), 3.23 (dd, J=28.2, 10.2 Hz, 2H), 2.65 (q, J=10.1 Hz, 2H), 0.91 (s, 9H), 0.10 (d, J=5.8 Hz, 6H) ppm. ¹³C NMR (126 MHz, CDCl₃) δ 163.4, 150.1, 140.2, 101.8, 88.4, 84.1, 82.1, 69.4, 68.9, 66.4, 58.6, 56.4, 25.8, 18.3, −4.4, −4.7 ppm. HRMS calc. for C₂₀H₃₆N₃O₇Si [M+H]⁺ 458.2323, found 458.2315.

### 1-[(2R,5R)-4-hydroxy-3-methoxy-5-(1-piperidyloxymethyl)tetrahydrofuran-2-yl]pyrimidine-2,4-dione (12)

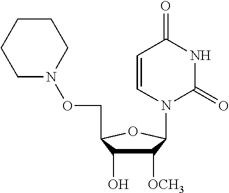

To a solution of 10 (0.30 g, 0.66 mmol) in THF (5 mL) at 25° C., tetrabutylammonium fluoride, 1M in THF (0.99 mmol, 0.99 mL) was added slowly in single portion and then stirred for 5 hr. Volatile matters were removed in high vacuum pump and crude residue thus obtained was purified by column chromatography (gradient: 10-60% EtOAc in hexane) to afford 12 (0.17 g, 76% yield). ¹H NMR (400 MHz, CDCl₃) δ 8.79 (s, 1H), 7.97 (d, J=8.2 Hz, 1H), 5.94 (d, J=2.2 Hz, 1H), 5.71 (dd, J=8.2, 1.8 Hz, 1H), 4.20 (td, J=7.4, 5.2 Hz, 1H), 4.16-4.11 (m, 1H), 4.08 (dt, J=7.1, 2.6 Hz, 1H), 3.95 (dd, J=11.2, 2.7 Hz, 1H), 3.77 (dd, J=5.2, 2.3 Hz, 1H), 3.61 (s, 3H), 3.36 (s, 2H), 2.84 (d, J=7.7 Hz, 1H), 2.39 (t, J=11.4 Hz, 2H), 1.76 (d, J=13.0 Hz, 2H), 1.59 (brs, 2H) ppm. ¹³C NMR (101 MHz, CDCl₃) δ 163.2, 150.2, 140.2, 102.0, 87.6, 84.0, 83.1, 69.5, 69.1, 58.8, 57.0, 56.7, 25.5, 23.5 ppm. HRMS calc. for C₁₅H₂₄N₃O₆ [M+H]⁺ 342.1665, found 342.1656.

### Synthesis of 1-[(2R,5R)-4-hydroxy-3-methoxy-5-(morpholinooxymethyl) tetrahydrofuran-2-yl]pyrimidine-2,4-dione (13)

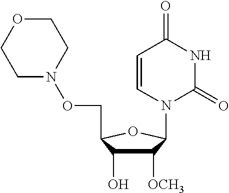

To a solution of 11 (0.85 g, 1.86 mmol) in THF (15 mL) at 22° C., tetrabutylammonium fluoride, 1M in THF (2.41 mmol, 2.41 mL) was added slowly in single portion and then stirred for 3 hr. Volatile matters were removed in high vacuum pump and crude residue thus obtained was purified by column chromatography (gradient: 0-5% MeOH in DCM) to afford 13 (0.52 g, 81% yield) as white solid. ¹H NMR (500 MHz, CDCl₃) δ 9.26 (s, 1H), 7.85 (d, J=8.2 Hz, 1H), 5.93 (d, J=2.1 Hz, 1H), 5.73 (d, J=8.2 Hz, 1H), 4.23-4.14 (m, 2H), 4.08 (dt, J=7.3, 2.9 Hz, 1H), 3.98 (dd, J=11.3, 3.2 Hz, 1H), 3.92 (d, J=11.7 Hz, 2H), 3.81-3.71 (m, 1H), 3.62 (s, 5H), 3.25 (t, J=9.1 Hz, 2H), 2.81 (d, J=8.3 Hz, 1H), 2.67 (td, J=10.9, 3.2 Hz, 2H) ppm. ¹³C NMR (101 MHz, CDCl₃) δ 163.4, 150.2, 140.0, 102.1, 87.7, 83.7, 82.7, 69.6, 68.9, 66.3, 58.9, 56.5, 56.23 ppm. HRMS calc. for C₁₄H₂₂N₃O₇ [M+H]⁺ 344.1458, found 344.1465.

### Synthesis of 3-[(diisopropylamino)-[(2R,5R)-5-(2,4-dioxopyrimidin-1-yl)-4-methoxy-2-(1-piperidyloxy methyl)tetrahydrofuran-3-yl]oxy-phosphanyl]oxypropanenitrile (14)

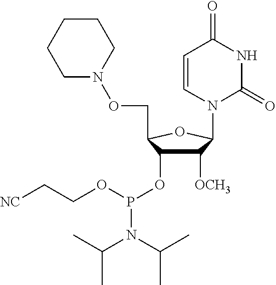

To a clear solution of 12 (0.60 g, 1.76 mmol) in DCM (20 mL), diisopropylethylamine (1.15 g, 8.79 mmol, 1.55 mL) and N-methylimidazole (0.51 g, 6.15 mmol, 0.49 mL) were added at 22° C. To this reaction mixture, 2-cyanoethyl-N,N-diisopropylchlorophosphoramidite (0.88 g, 3.52 mmol, 0.82 mL) was added slowly after 5 minutes and stirred for 0.5 hr. Reaction mixture was diluted with DCM (10 mL) and quenched with 10% NaHCO₃ solution (20 mL). Organic layer was separated, dried on anhydrous Na₂SO₄, filtered and filtrate was evaporated to dryness. The crude compound was thus obtained was purified by column chromatography (gradient: 20-80% EtOAc in hexane) to afford 14 (0.66 g, 70% yield) as hygroscopic solid. ¹H NMR (400 MHz, CDCl₃) δ 8.81 (s, 1H), 7.96 (dd, J=10.9, 8.1 Hz, 1H), 5.97 (d, J=3.7 Hz, 1H), 5.69 (d, J=8.1 Hz, 1H), 4.49-4.16 (m, 2H), 4.09 (td, J=11.2, 2.3 Hz, 1H), 3.98-3.77 (m, 4H), 3.72-3.58 (m, 2H), 3.51 (d, J=14.2 Hz, 3H), 3.39-3.23 (m, 2H), 2.64 (dt, J=11.9, 6.3 Hz, 2H), 2.39 (d, J=10.5 Hz, 2H), 1.73 (s, 2H), 1.58 (s, 3H), 1.30-1.06 (m, 15H) ppm. ¹³C NMR (101 MHz, CDCl₃) δ 163.4, 163.4, 150.4, 150.3, 140.4, 140.2, 117.8, 117.6, 102.1, 102.0, 87.8, 87.5, 83.5, 83.4, 83.1, 83.1, 82.4, 82.4, 82.2, 82.2, 70.9, 70.7, 70.1, 70.0, 69.6, 69.5, 58.9, 58.7, 58.7, 58.3, 58.3, 58.2, 58.1, 58.0, 56.9, 53.6, 43.6, 43.5, 43.4, 43.4, 25.4, 24.8, 24.7, 24.7, 24.7, 24.7, 23.5, 23.4, 20.6, 20.5, 20.5, 20.5 ppm. ³¹P NMR (162 MHz, CDCl₃) δ 150.98, 150.54 ppm. HRMS calc. for C₂₄H₄₁N₅O₇P [M+H]⁺ 542.2744, found 542.2747.

### Synthesis of 3-[(diisopropylamino)-[(2R,5R)-5-(2,4-dioxopyrimidin-1-yl)-4-methoxy-2-(morpholinooxymethyl)tetrahydrofuran-3-yl]oxy-phosphanyl]oxypropane nitrile (15)

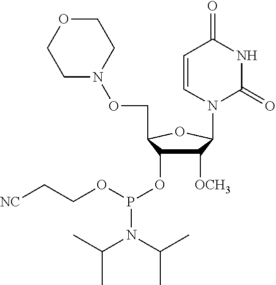

To a solution of 13 (0.3 g, 0.87 mmol) in dry acetonitrile (10 mL) was added 5-(ethylthio)-1H-tetrazole (0.12 g, 0.87 mmol). 2-Cyanoethyl-N,N,N′,N′-tetraisopropylphosphordiamidite (1.14 mmol, 0.37 mL) was added slowly to the reaction mixture and stirred at 22° C. for 3 hr. The reaction mixture was filtered, volatile matters were removed under high vacuum pump and residue was purified by flash column chromatography using a gradient of EtOAc in hexane containing 0.2% triethylamine to yield 15 (0.31 g, 65% yield) as white solid. To remove P (V) impurity from the column purified compound, 15 was dissolved in methyl tert-butylether (MTBE) (25 mL) and washed with 50% DMF in water (2×10 mL) and the with brine (3×20 mL). Organic layer was separated, dried over anhydrous Na₂SO₄, filtered and filtrate was evaporated under high vacuum pump to obtain 15 as white foam. ¹H NMR (400 MHz, CD₃CN) δ 8.94 (s, 1H), 7.76 (dd, J=8.9, 8.2 Hz, 1H), 5.88 (dd, J=7.3, 4.6 Hz, 1H), 5.65 (dd, J=8.2, 3.0 Hz, 1H), 4.50-4.14 (m, 2H), 4.08-3.98 (m, 1H), 3.92-3.74 (m, 5H), 3.65 (dtd, J=10.3, 6.8, 4.7 Hz, 2H), 3.56-3.36 (m, 6H), 3.21 (d, J=10.2 Hz, 2H), 2.75-2.51 (m, 4H), 1.28-1.10 (m, 17H) ppm. ¹³C NMR (126 MHz, CD₃CN) δ 163.9, 163.9, 151.4, 151.4, 141.0, 141.0, 119.6, 119.6, 102.8, 102.7, 88.4, 87.9, 83.5, 83.5, 83.2, 83.2, 83.2, 83.1, 82.7, 82.7, 72.2, 72.0, 71.6, 71.5, 71.0, 70.9, 66.8, 66.8, 59.8, 59.6, 59.2, 59.1, 58.9, 58.9, 58.6, 58.6, 57.2, 57.0, 49.5, 44.2, 44.2, 44.1, 44.1, 27.2, 25.0, 25.0, 24.9, 24.9, 24.9, 24.8, 21.1, 21.0, 21.0 ppm. ³¹P NMR (202 MHz, CD₃CN) δ 151.48, 151.16 ppm HRMS calc. for C₂₃H₃₈N₅O₈PNa [M+Na]⁺ 566.2356, found 566.2379.

Synthesis of Oligonucleotides from Modified Morpholino Building Blocks

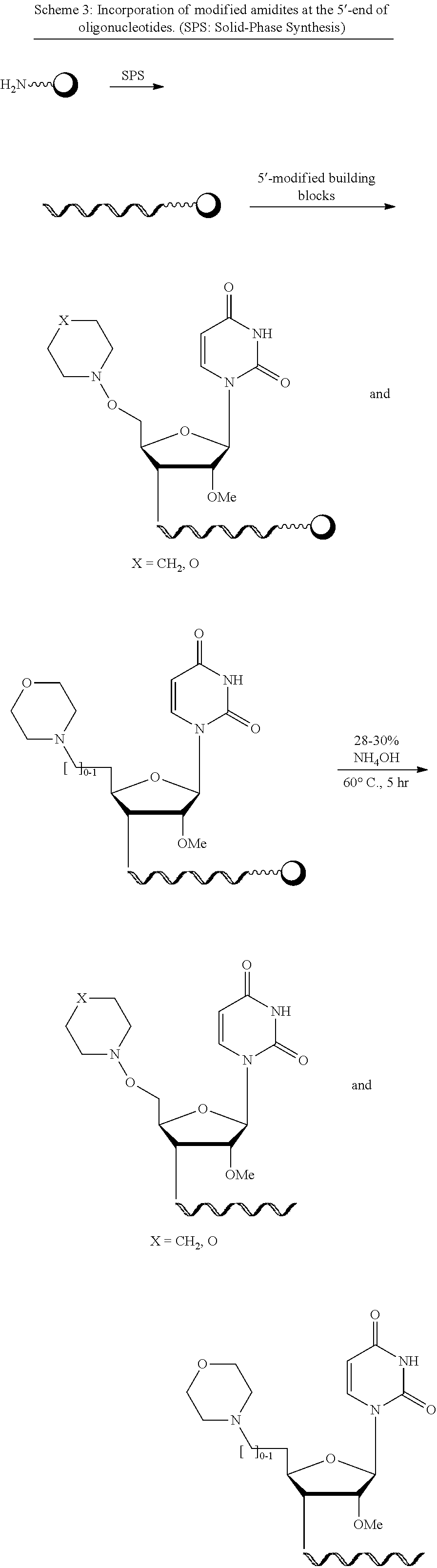

### Oligonucleotide Synthesis and Purification

Oligonucleotides were synthesized on K&A H-8-SE at 40-μmol scale using universal supports. A solution of 0.25 M 5-(S-ethylthio)-1H-tetrazole in acetonitrile (CH₃CN) was used as the activator. The solutions of commercially available phosphoramidites and synthesized phosphoramidities were used at 0.15 M in anhydrous CH₃CN or CH₂Cl₂. The oxidizing reagent was 0.02 M I₂ in THF/pyridine/H₂O. N,N-Dimethyl-N′-(3-thioxo-3H-1,2,4-dithiazol-5-yl) methanimidamide (DDTT), 0.1 M in pyridine, was used as the sulfurizing reagent. The detritylation reagent was 3% dichloroacetic acid in CH₂Cl₂. Waiting time for coupling, capping, oxidation, and sulfurization step are 450s, 25s, 80s and 300s respectively. After completion of the automated synthesis, the oligonucleotide was manually released from support and deprotected using 28-30% ammonium hydroxide solution at 60° C. for 5 h.

After filtration through a 0.45-μm nylon filter, oligonucleotides were purified by ion exchange and/or reverse phase column chromatography. For ion exchange, preparative HPLC custom packed with TSKGel SuperQ-5 PW (20) (Sigma) using an appropriate gradient of mobile phase (buffer A: 20 mM sodium phosphate, 15% CH₃CN, pH 8.5; buffer B: 1 M NaBr, 20 mM sodium phosphate, 15% CH₃CN, pH 8.5) and desalted using size-exclusion chromatography using a custom packed with Sephadex G25 (GE Healthcare) and water as an eluent. Oligonucleotides were then quantified by measuring the absorbance at 260 nm. Extinction coefficients were calculated using the following extinction coefficients for each residue: A, 13.86; T/U, 7.92; C, 6.57; and G, 10.53 M-1 cm-1. The purity and identity of modified ONs were verified by analytical reRP-HPLC chromatography and mass spectrometry, respectively.

### HPLC Conditions

For ON1-ON16 buffer A: 95 mM hexafluoroisopropanol, 16.3 mM TEA, 0.05 mM EDTA; buffer B: MeOH gradient 2-29% B for 39 min.

TABLE 11
Sequences and mass spectroscopy
characterization of target sequence
using morpholino-conjugated
building blocks

| | Sense and | SEQ | | | | |
|---|---|---|---|---|---|---|
| En- | antisense | ID | Tar- | S/ | Mass (M-H)⁻ | |
| tryª | strand (5′-3′)^{b} | NO | get | AS | Calcd. | Obsd. |
| ON1 | Mo1•g•UgAcAaAUA | 160 | Apob | S | 8795.45 | 8796.14 |
| | uGgGcAuCaAL | | | | | |
| ON2 | Mo2•g•UgAcAaAUA | 161 | Apob | S | 8809.48 | 8809.84 |
| | uGgGcAuCaAL | | | | | |
| ON3 | Pip•g•UgAcAaAUA | 162 | Apob | S | 8809.48 | 8809.76 |
| | uGgGcAuCaAL | | | | | |
| ON4 | Mo3•g•UgAcAaAUA | 163 | Apob | S | 8811.45 | 8811.86 |
| | uGgGcAuCaAL | | | | | |
| ON5 | u•g•UgAcAaAUAuG | 164 | Apob | S | 8726.35 | 8726.92 |
| | gGcAuCaAL | | | | | |
| ON6 | u•U•gAuGcCcAuau | 165 | Apob | AS | 7530.89 | 7531.36 |
| | UuGuCaCa•a•a | | | | | |
| ON7 | Mo1•U•gAuGcCcAu | 166 | Apob | AS | 7599.99 | 7600.36 |
| | auUuGuCaCa•a•a | | | | | |
| ON8 | Mo2•U•gAuGcCcAu | 167 | Apob | AS | 7614.02 | 7613.96 |
| | auUuGuCaCa•a•a | | | | | |
| ON9 | Mo2•u•gAuGcCcAu | 168 | Apob | As | | |
| | auUuGuCaCa•a•a | | | | | |
| ON10 | Pip•U•gAuGcCcAu | 169 | Apob | AS | 7614.02 | 7614.23 |
| | auUuGuCaCa•a•a | | | | | |
| ON11 | Mo3•U•gAuGcCcAu | 170 | Apob | AS | 7615.99 | 7616.39 |
| | auUuGuCaCa•a•a | | | | | |
| ON12 | u•U•auaGagcaaga | 171 | TTR | AS | 7655.12 | 7655.90 |
| | AcAcuguu•u•u | | | | | |
| ON13 | VPu•U•auaGagcaa | 172 | TTR | AS | 7731.12 | 7730.80 |
| | gaAcAcuguu•u•u | | | | | |
| ON14 | Mo1•U•auaGagcaa | 173 | TTR | AS | 7724.22 | 7724.68 |
| | gaAcAcuguu•u•u | | | | | |
| ON15 | Mo2•U•auaGagcaa | 174 | TTR | AS | 7738.25 | 7736.78 |
| | gaAcAcuguu•u•u | | | | | |
| ON16 | Pip•U•auaGagcaa | 175 | TTR | AS | 7738.25 | 7738.46 |
| | gaAcAcuguu•u•u | | | | | |
| ON17 | Mo3•U•auaGagcaa | 176 | TTR | AS | 7740.22 | 7739.79 |
| | gaAcAcuguu•u•u | | | | | |

^{a}ON12 and ON13 were synthesized following the previously reported procedure⁴
^{b}Chemical modifications are indicated as follows: VP, vinylphosphonate;
•, PS linkage; lower case, 2-OMe; italicized upper case, 2′-F; L, trivalent-GalNAc respectively. Structures of Mo1, Mo2, Mo3, Pip and L are shown in FIG. 33.

### ApoB Assay:

All studies were conducted using protocols consistent with local, state, and federal regulations, as applicable, and were approved by the Institutional Animal Care and Use Committee (IACUC) at Alnylam Pharmaceuticals. Only female C57BL/6 mice (Charles River Laboratories) of 6 −8 weeks old mice used. Mice were received subcutaneous administration of test article solutions at a dose volume of 10 μL/g. There are 3 mice for each group and mice were gave a single subcutaneous (s.c.) administration of siRNA at 3 mg/kg at day 0. Plasma samples were collected by using EDTA collection tube at days 0 (pre-dose), 7, 14, and 21. Mouse Apo-B protein levels were determined using Mouse Apo B SimpleStep ELISA® Kit (Abcma; cat, No. ab230932), in accordance with the manufacturer's protocol, and data were normalized to pre-bleed target protein levels.

TABLE 12
Duplexes for in vivo silencing

| | Sense strand (upper) and | SEQ |
|---|---|---|
| Duplex | antisense strand | ID |
| ID | (lower)ª (5′-3′) | NO |
| I | Mo1•g•UgAcAaAUAuGgGcAuCaAL | 177 |
| | u•U•gAuGcCcAuauUuGuCaCa•a•a | 178 |
| parent | u•g•UgAcAaAUAuGgGcAuCaAL | 179 |
| | u•U•gAuGcCcAuauUuGuCaCa•a•a | 180 |
| II | u•g•UgAcAaAUAuGgGcAuCaAL | 181 |
| | Mo1•U•gAuGcCcAuauUuGuCaCa•a•a | 182 |
| III | Mo2•g•UgAcAaAUAuGgGcAuCaAL | 183 |
| | u•U•gAuGcCcAuauUuGuCaCa•a•a | 184 |
| IV | Pip•g•UgAcAaAUAuGgGcAuCaAL | 185 |
| | u•U•gAuGcCcAuauUuGuCaCa•a•a | 186 |
| V | Mo3•g•UgAcAaAUAuGgGcAuCaAL | 187 |
| | u•U•gAuGcCcAuauUuGuCaCa•a•a | 188 |
| VI | u•g•UgAcAaAUAuGgGcAuCaAL | 189 |
| | Mo2•UsgAuGcCcAuauUuGuCaCa•a•a | 190 |
| VII | u•g•UgAcAaAUAuGgGcAuCaAL | 191 |
| | Pip•U•gAuGcCcAuauUuGuCaCa•a•a | 192 |
| VIII | u•g•UgAcAaAUAuGgGcAuCaAL | 193 |
| | Mo3•U•gAuGcCcAuauUuGuCaCa•a•a | 194 |
| si-5 | u•g•UgAcAaAUAuGgGcAuCaAL | 195 |
| | Mo2•u•gAuGcCcAuauUuGuCaCa•a•a | 196 |
| si-6 | u•g•UgAcAaAUAuGgGcAuCaAL | 197 |
| | u•u•gAuGcCcAuauUuGuCaCa•a•a | 198 |
| si-7 | u•g•UgAcAaAUAuGgGcAuCaAL | 199 |
| | Mo2•U•gAuGcCcAuauUuGuCaCa•a•a | 200 |
| si-8 | Mo2•G•UgAcAaAUAuGgGcAuCaAL | 201 |
| | u•U•gAuGcCcAuauUuGuCaCa•a•a | 202 |
| si-9 | Mo2•g•UgAcAaAUAuGgGcAuCaAL | 203 |
| | u•U•gAuGcCcAuauUuGuCaCa•a•a | 204 |
| si-10 | u•g•UgAcAaAUAuGgGcAuCaAL | 205 |
| | u•U•gAuGcCcAuauUuGuCaCa•a•a | 206 |

^{a}Chemical modifications are indicated as follows:
•, PS linkage; lower case, 2′-OMe; upper case, 2′-F;
L, trivalent-GalNAc respectively. Structures of Mo1, Mo2, Mo3, Pip and L are shown in FIG. 33.

### On- and Off-Target Activity Determination (Luciferase Reporter Assay):

COS-7 cells were cultured at 37° C., 5% CO₂ in Dulbecco's Modified Eagle Medium supplemented with 10% fetal bovine serum. Cells were co-transfected in 96-well plates (15,000 cells/well) with 10 ng luciferase reporter plasmid and 0.64 pM to 50 nM siRNA in 5-fold dilutions using 2 μL Lipofectamine 2000 (Thermo Fisher Scientific) according to manufacturer's instructions. Cells were harvested at 48 hours after transfection for the dual luciferase assay (Promega) according to manufacturer's instructions. The on-target reporter plasmid contained a single site perfectly complementary to the antisense strand in the 3′ untranslated (3′ UTR) of Renilla luciferase. The off-target reporter plasmid contained four tandem seed-complementary sites separated by a 19-nucleotide spacer (TAATATTACATAAATAAAA) in the 3′ UTR of Renilla luciferase. Both plasmids co-expressed firefly luciferase as a transfection control.

Primary rat hepatocytes (BioreclamationIVT) were seeded in 96-well collagen I pre-coated plates (Gibco) at approximately 50,000 cells/well in 95 μL INVITROGRO CP Rodent Medium (BioreclamationIVT). Pre-incubated lipid/siRNA complex (0.25 μL RNAiMax (Thermo Fisher Scientific) and 1 μL siRNA in 3.75 μL Opti-MEM for 15 min) was added to transfect the cells and incubated for 48 h at 37° C. in an atmosphere of 5% CO₂. The final concentration of the siRNA was 50 nM, and each siRNA was tested in quadruplicate. The media was removed, RNA was extracted using the miRNeasy 96 kit (Qiagen), cDNA library was prepared with the TruSeq Stranded Total RNA Library Prep Kit (Illumina) and sequenced on the HiSeq or NextSeq500 sequencers (Illumina), all according to manufacturers' instructions. Raw RNAseq reads were filtered with minimal mean quality scores of 25 and minimal remaining length of 36, using fastq-mcf. Filtered reads were aligned to the Rattus norvegicus genome (Rnor_6.0) using STAR (ultrafast universal RNAseq aligner) with default parameters. Uniquely aligned reads were counted by feature Counts.⁵ Differential gene expression analysis was performed using the R package DESeq2.

TABLE 13
IC₅₀ data on the on-target activity in luciferase reporter assay

| | IC50 | |
|---|---|---|
| siRNA | (nM) | IC50 change (fold to parent) |
| ON11 (parent) | 0.0075 | 1.0 |
| ON13 (Mo1) | 0.0961 | 13 |
| ON14 (Mo2) | 0.2216 | 30 |
| ON15 (Pip) | 0.0102 | 1.4 |
| ON16 (Mo3) | 0.0107 | 1.4 |

In Vitro Relative hAgo2 Binding Assay:

Two and a half μg of anti-FLAG M2 antibody was incubated with 20 ul of Dynabeads® Protein G (Life Technologies) in phosphate buffered saline supplemented with 0.02% Tween-20. After washing in fresh buffer, 4 μg of N-terminal FLAG-tagged recombinant human Ago2 (Active Motif) was incubated for 10 minutes at room temperature with gentle rotation, and unbound protein was removed by washing in 1×PBS. Beads were resuspended in Ago2 binding and wash buffer (150 mM NaCl, 20 mM Tris pH 8.0, 2 mM MgCl2, 0.5 mM TCEP) Antisense RNAs were added to hAgo2 protein immobilized on Dynabeads to a final concentration of 0.05 μg in 40 μl final volume and allowed to incubate for 1 hour at 37° C. After washing beads 3 times in 1 ml PBST (0.25% Triton X-100), total loaded AS RNA was quantified using stem-loop RT-qPCR as previously described.⁶ Data represents N=3 replicate measurements.

### REFERENCES

1. M. J. Fer, P. Doan, T. Prange, S. Calvet-Vitale and C. Gravier-Pelletier, J. Org. Chem., 2014, 79, 7758-7765.
2. A. Varizhuk, A. Chizhov, I. Smirnov, D. Kaluzhny and V. Florentiev, Eur. J. Org. Chem., 2012, 2173-2179.
3. M. Israel and R. J. Murray, J. Med. Chem., 1982, 25, 24-28.
4. P. Kumar, R. G. Parmar, C. R. Brown, J. L. S. Willoughby, D. J. Foster, I. R. Babu, S. Schofield, V. Jadhav, K. Charisse, J. K. Nair, K. G. Rajeev, M. A. Maier, M. Egli and M. Manoharan, Chem. Commun., 2019, 55, 5139-5142.
5. Y. Liao, G. K. Smyth and W. Shi, Bioinformatics, 2014, 30, 923-930.
6. C. Chen, D. A. Ridzon, A. J. Broomer, Z. Zhou, D. H. Lee, J. T. Nguyen, M. Barbisin, N. L. Xu, V. R. Mahuvakar, M. R. Andersen, K. Q. Lao, K. J. Livak and K. J. Guegler, Nucleic Acids Res., 2005, 33, e179-e179.

### Example 3: Synergy Between Modifications at 5′-End of Sense and Antisense Strand Improves Efficacy of siRNAs

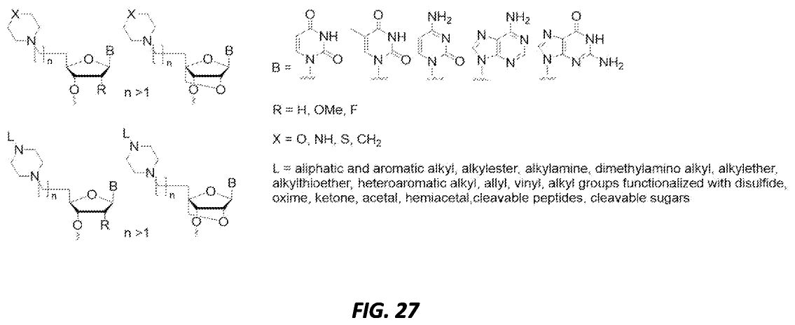

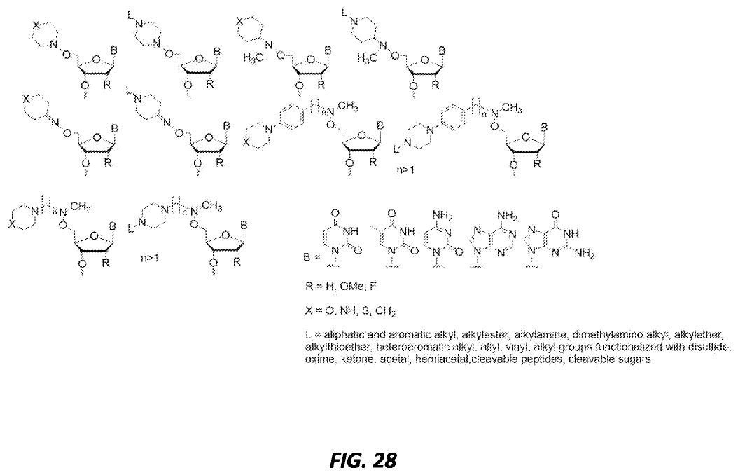

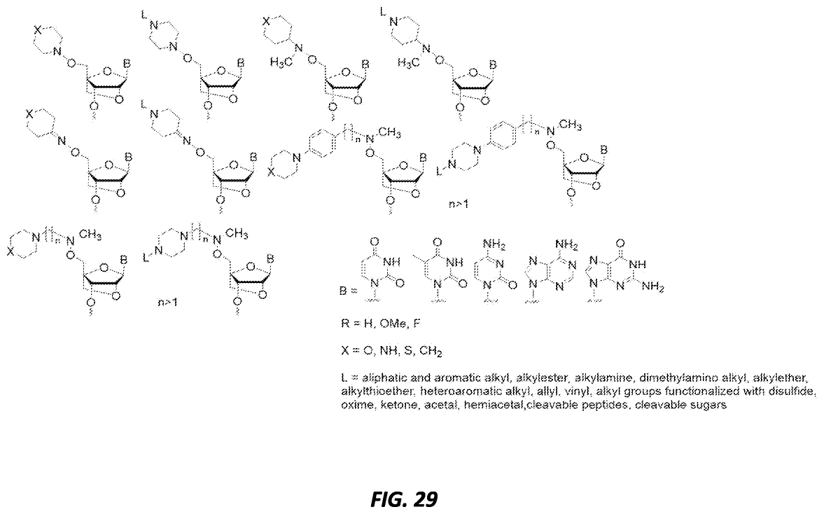

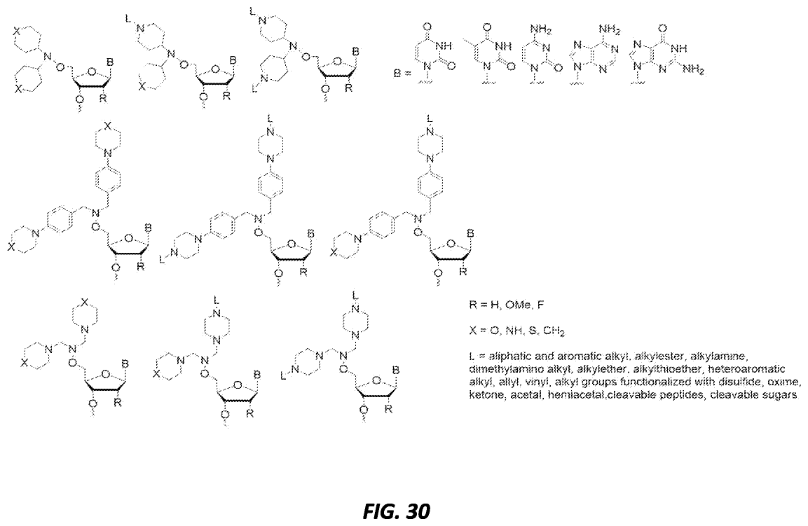

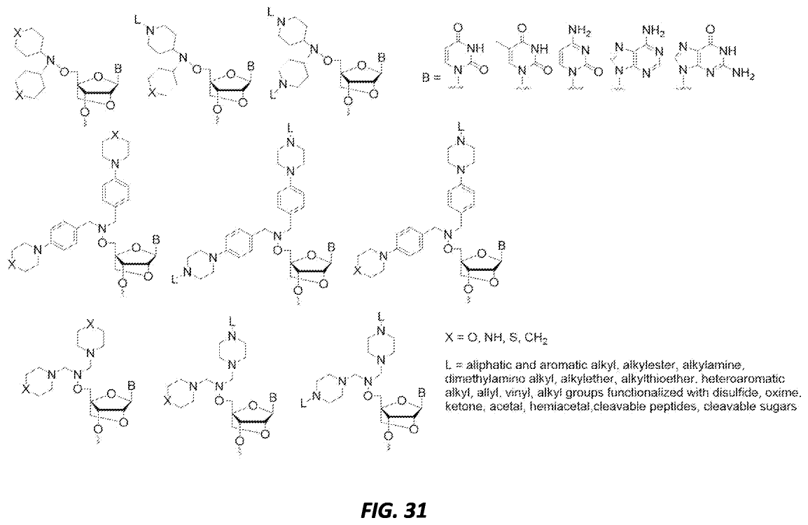

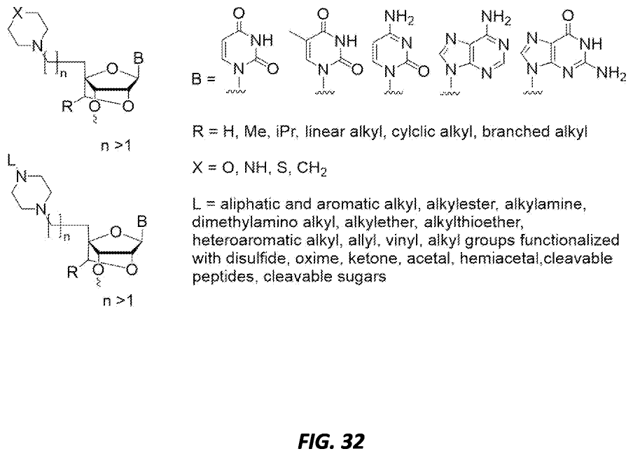

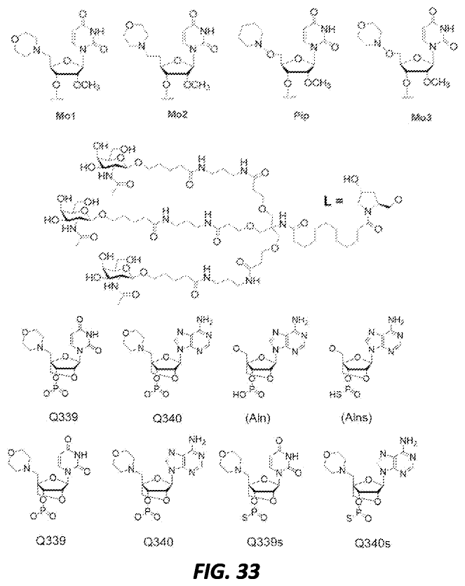

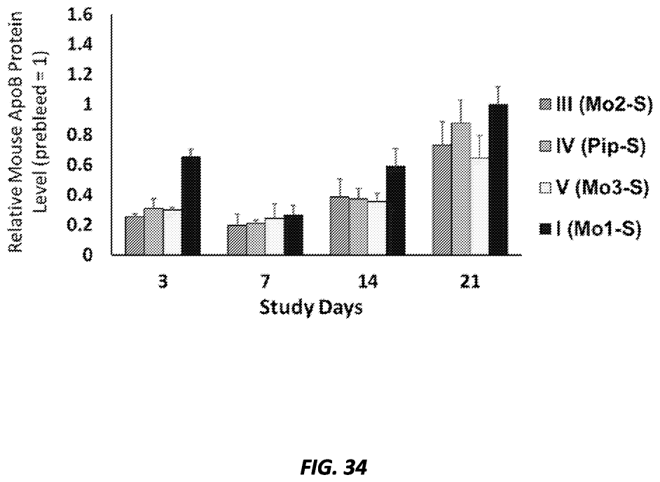

Year 2018 mark the beginning of short RNA duplexes as a new class of medicines. These short duplexes are often 21-mer long with a 2-nucleotide overhang and are knows as small interfering RNAs (siRNAs). siRNAs take advantage RNA interference (RNAi) pathway, an endogenous mechanism used by cells to control gene expression. Exogeneous siRNAs when administrated to cells interact with RNA Induced Silencing Complex (RISC) complex which retains one strand (antisense strand) and removes other strand (sense strand). RISC complex with antisense strand then target mRNA of intended gene and halts the gene expression. Various factors such as thermodynamic asymmetry between two ends of siRNA, identity of first nucleotide at 5′-end determines the selection of antisense strand. However, use of modified nucleotide at the 5′-end to induce strand bias is known. In this regard, we recently showed that presence of 5′-morpholino-2′-OMe nucleotide at the 5′-end improves strand selection and RNAi activity by blocking loading of sense strand into RISC. In a complementary approach, we have shown that the loading of the antisense strand into active RISC can be improved by modification of the 5′ terminus with (E)-vinylphosphonate (E-VP); E-VP acts as a bioisostere of the natural 5′-monophosphate. 5′-monophosphate plays an important role in the strand selection through interactions in mid-domain. Thus, installing a VP (a mimic of monophosphate) at the 5′-end of antisense strand and a morpholino ring at the 5′-end of sense strand would tilt the strand bias completely in favor of loading of antisense strand loading. Locked nucleic acids (LNA) binds strongly to target RNA, improves stability against nucleases. These two factors could be advantageous in siRNA design as LNA on 5′-end can modulate thermodynamic asymmetry between two ends (making the end with 5′-end of sense strand more stable) and can also improve metabolic stability of sense strand. LNA in combination with morpholino at 5′-end will strongly disfavor the loading of sense strand. We present synthesis of new 5′-morpholino LNA building blocks (FIG. 27) with the aim of optimizing RNAi activity. Our goals are to 1) block the loading of sense strand by preventing phosphorylation (5′-morpholino), 2) improve metabolic stability against 5′-exonucleases (LNA), and 3) promote loading of antisense strand (5′-E-VP). Using this strategy, metabolically stable and active siRNA are obtained.

### Materials and Methods:

General: Commercially available starting materials, reagents, and solvents were used as received. All moisture-sensitive reactions were carried under anhydrous conditions under argon atmosphere. Flash chromatography was performed on a Teledyne ISCO Combi Flash system using pre-packed ReadySe.p Teledyne ISCO silica gel columns. TLC was performed on Merck silica-coated plates 60 F₂₅₄. Compounds were visualized under UV light (254 nm) or after spraying with the p-anisaldehyde staining solution followed by heating. ESI-HRMS spectra were recorded on Waters QTof API US spectrometer using the direct flow injection in the positive mode (capillary=3000 kV, cone=35, source temperature=120° C., and desolvation temperature=350° C.). ¹H and ¹³C NMR spectra were recorded at room temperature on Varian spectrometers, and chemical shifts in ppm are referenced to the residual solvent peaks. Coupling constants are given in Hertz. Signal splitting patterns are described as singlet(s), doublet (d), triplet (t), quartet (q), broad signal (br), or multiplet (m). ³¹P NMR spectra were recorded under proton-decoupled mode; chemical shifts are referenced to external H₃PO₄ (80%).

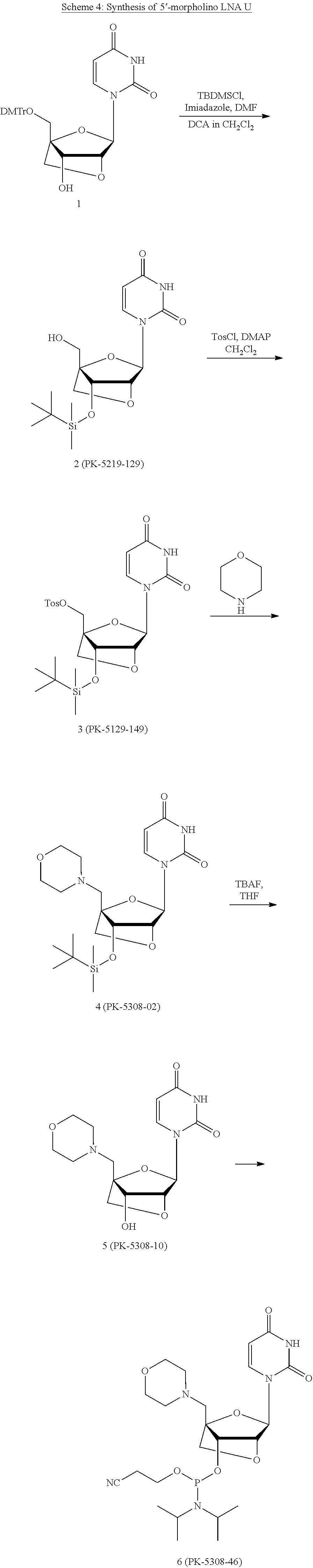

### Synthesis of nucleoside 2 (PK-5219-129)

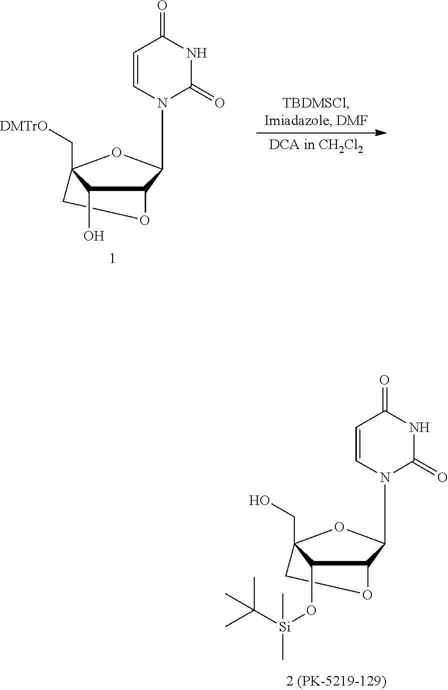

Nucleoside 1 (11.0 g, 19.7 mmol) was dissolved in dry DMF (30 mL). To this was added imidazole (1.70 g, 24.9 mmol) followed by tert-butyldimethylsilyl chloride (3.6 g, 23.8 mmol) and the mixture was stirred at room temperature for 18 h. The reaction mixture was diluted with EtOAc (200 mL) and washed with saturated aqueous solution of NaHCO₃ (2×50 mL). The organic phase was dried (MgSO₄) and concentrated under reduced pressure. The crude was purified by column chromatography using a gradient of 0-70% EtOAc in hexane to obtain fully protected nucleoside (10.7 g). To this was added a solution of dichloroacetic acid in CH₂Cl₂ (3% wt/v, 120 mL). The reaction mixture was stirred for 1 h whereupon MeOH (5 mL) was added and stirring was continued for another 20 minutes. The solvents were removed, and the residue was purified by column chromatography using a gradient of 0-12% MeOH in CH₂Cl₂ to afford PK-5219-129 (4.95 g, 70%). ¹H NMR (400 MHz, DMSO) δ 11.36 (s, 1H), 7.74 (d, J=8.1 Hz, 1H), 5.63 (dd, J=8.1, 2.2 Hz, 1H), 5.44 (d, J=0.7 Hz, 1H), 5.20 (t, J=5.3 Hz, 1H), 4.14 (s, 1H), 4.00 (s, 1H), 3.79 (d, J=7.7 Hz, 1H), 3.76-3.67 (m, 2H), 3.65 (d, J=7.8 Hz, 1H), 0.84 (s, 9H), 0.07 (s, 3H), 0.05 (s, 3H).

### Synthesis of nucleoside 3 (PK-5308-149)

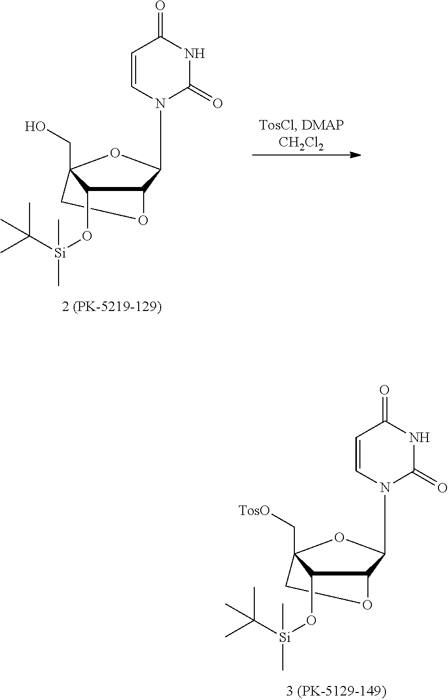

To a solution of 2 (PK-5219-129) (2.0 g, 5.39 mmol) in CH₂Cl₂ was added 4-dimethylaminopyridine (DMAP, 1.32 g, 10.78 mmol) and 4-toluenesulfonyl chloride (TosCI, 1.28, 6.74 mmol) at 0° C. The reaction mixture was slowly warm to room temperature and stirred for 2 h. The reaction mixture was diluted with CH₂Cl₂ (100 mL) and washed with saturated aqueous solution of NaHCO₃ (50 mL). The aqueous phase was back extracted with CH₂Cl₂ (50 mL), and combined organic phase was dried (MgSO₄) and concentrated at reduced pressure. The residue was purified by column chromatography using a gradient of 0-4% MeOH in CH₂Cl₂ to afford nucleoside 3 (PK-5219-149) (2.15 g, 75%). MS (ESI⁺) m/z calcd for C₂₃H₃₃N₂O₈SSi [M+H]⁺ 525.1727, found 525.1724. ¹H NMR (400 MHz, DMSO) δ 11.37 (d, J=2.1 Hz, 1H), 7.86-7.73 (m, 2H), 7.51-7.49 (m, 3H), 5.56 (dd, J=8.1, 2.2 Hz, 1H), 5.45 (s, 1H), 4.62 (d, J=11.8 Hz, 1H), 4.23-4.20 (d, 2H), 4.00 (s, 1H), 3.79-3.63 (m, 2H), 2.41 (s, 3H), 0.75 (s, 9H), 0.02 (s, 3H), 0.01 (s, 3H). ¹³C NMR (126 MHz, DMSO) δ 163.17, 149.90, 145.43, 138.95, 131.44, 130.29, 127.71, 101.00, 86.84, 85.26, 78.65, 70.96, 70.82, 66.03, 25.29, 21.07, 17.41, −5.00, −5.49.

### Synthesis of nucleoside 4 (PK-5308-02)

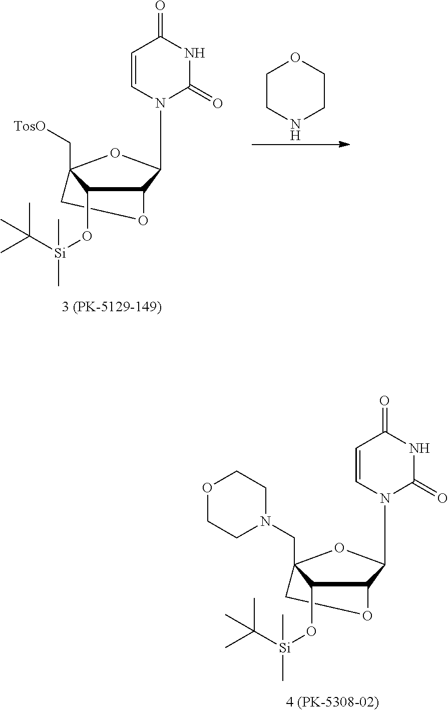

Nucleoside 3 (PK-5308-149) (1.80 g, 3.43 mmol) was dissolved in morpholine (15 mL). The reaction mixture was stirred at 60° C. for 40 h. The solvent was removed, and the residue was dissolved in EtOAc (100 mL) and washed with H2O (50 mL). The aqueous phase was back extracted with EtOAC (2×25 mL). The combined organic phase was dried (MgSO₄) and concentrated at reduced pressure. The crude was purified by column chromatography using a gradient of 0-6% MeOH in CH₂Cl₂ to afford 4 (PK-5308-02) (1.10 g, 73%). MS (ESI⁺) m/z calcd for C₂₀H₃₄N₃O₆Si [M+H]⁺ 440.2217, found 440.2220. ¹H NMR (500 MHz, DMSO) δ 11.35 (s, 1H), 7.71 (d, J=8.1 Hz, 1H), 5.66 (d, J=8.1 Hz, 1H), 5.46 (s, 1H), 4.15 (s, 1H), 3.94 (s, 1H), 3.81 (d, J=8.0 Hz, 1H), 3.72 (d, J=8.0 Hz, 1H), 3.57-3.55 (m, 4H), 2.74 (d, J=14.5 Hz, 1H), 2.68 (d, J=14.5 Hz, 1H), 2.57-2.53 (m, 2H), 2.47-2.43 (m, 2H), 0.84 (s, 9H), 0.06 (s, 6H). ¹³C NMR (126 MHz, DMSO) δ 163.22, 149.95, 139.11, 101.06, 88.41, 86.60, 78.25, 71.85, 71.22, 66.24, 54.87, 54.29, 25.44, 17.57, −4.84, −5.30.

### Synthesis of nucleoside 5 (PK-5308-10)

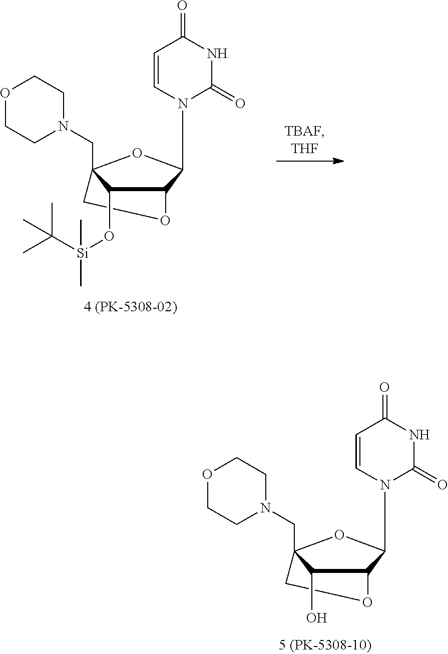 [SMILES: CC(C)(C)[Si](C)(C)O[C@@H]1C2OC[C@]1(CN1CCOCC1)O[C@H]2n1ccc(=O)[nH]c1=O ; O=c1ccn([C@@H]2O[C@@]3(CN4CCOCC4)COC2[C@H]3O)c(=O)[nH]1]

To a solution of 4 (PK-5308-02) (1.05 g, 2.38 mmol) in THF was added tetrabutylammonium fluoride (TBAF, 1M in THF, 3.60 mL, 3.60 mmol). The reaction mixture was stirred at room temperature for 1 h. The solvent was removed, and the crude was purified by column chromatography using a gradient of 0-5% MeOH in EtOAc to afford 5 (PK-5308-10) (0.70 g, 90%). MS (ESI⁺) m/z calcd for C₁₄H₂₀N₃O₆ [M+H]⁺ 326.1352, found 326.1360. ¹H NMR (500 MHz, DMSO) δ 11.34 (s, 1H), 7.76 (d, J=8.1 Hz, 1H), 5.77-5.71 (m, 1H), 5.65 (d, J=8.1 Hz, 1H), 5.42 (s, 1H), 4.12 (s, 1H), 3.84 (d, J=8.0 Hz, 1H), 3.77 (d, J=3.2 Hz, 1H), 3.70 (d, J=8.0 Hz, 1H), 3.56 (t, J=4.7 Hz, 4H), 2.74 (s, 2H), 2.65-2.54 (m, 2H), 2.48-2.38 (m, 2H). ¹³C NMR (126 MHz, DMSO) δ 163.20, 149.95, 139.19, 100.92, 88.40, 86.48, 78.58, 71.60, 69.91, 66.31, 54.78, 54.50.

### Synthesis of nucleoside 6 (PK-5308-46)

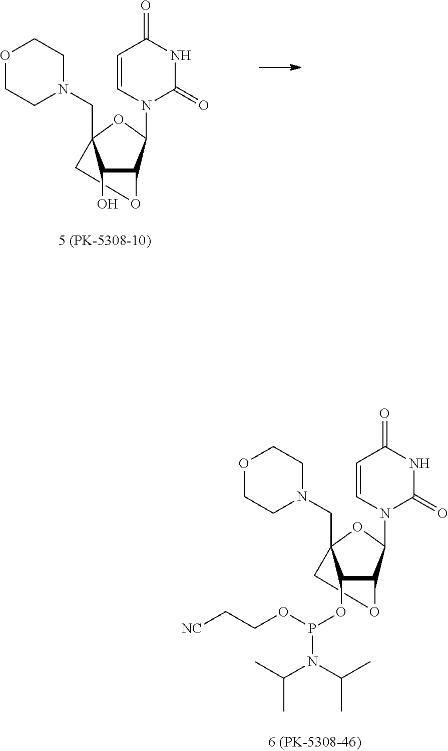 [SMILES: O=c1ccn([C@@H]2O[C@@]3(CN4CCOCC4)COC2[C@H]3O)c(=O)[nH]1 ; CC(C)N(C(C)C)P(OCCC#N)O[C@@H]1C2OC[C@]1(CN1CCOCC1)O[C@H]2n1ccc(=O)[nH]c1=O]

To a suspension of nucleoside 5 (PK-5308-10) (0.65 g, 2.0 mmol) in anhydrous CH₂Cl₂ (5 mL) was added 2-Cyanoethyl N, N,N′, N′-tetraisopropylphosphordiamidite (PN₂ 1.20 g, 4 mmol) followed by 4,5 dicyanoimidazole (DCI, 0.29 mg, 2.5 mmol). The reaction mixture was stirred at room temperature for 3 h whereupon it was diluted with CH₂Cl₂ (50 mL) and washed with saturated aqueous NaHCO₃ (25 mL). The aqueous phase was back extracted with CH₂Cl₂ (25 mL), and the combined organic phase was dried (MgSO₄) and concentrated at reduced pressure. The residue was purified by column chromatography using a gradient of 0-2% MeOH in CH₂Cl₂ (containing 0.2% Et₃N) to afford 6 (PK-5308-46) (0.88 g, 84%). MS (ESI⁺) m/z calcd for C₂₃H₃₇N₅O₇P [M+H]⁺ 526.2431, found 526.2439. ³¹P NMR (202 MHz, CD₃CN) δ 149.90, 149.58.

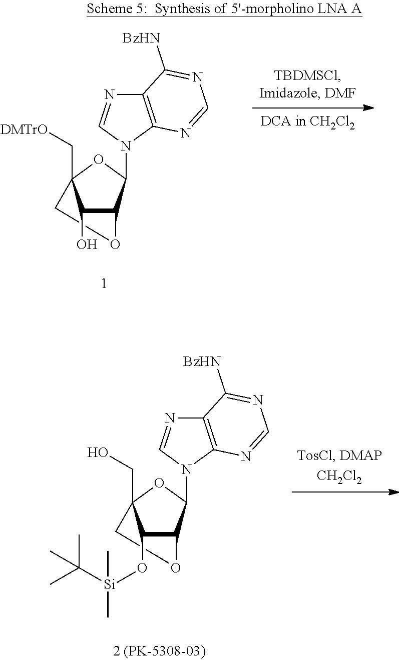

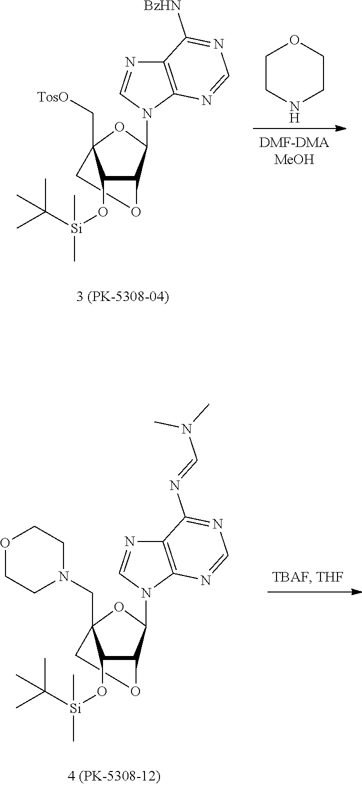

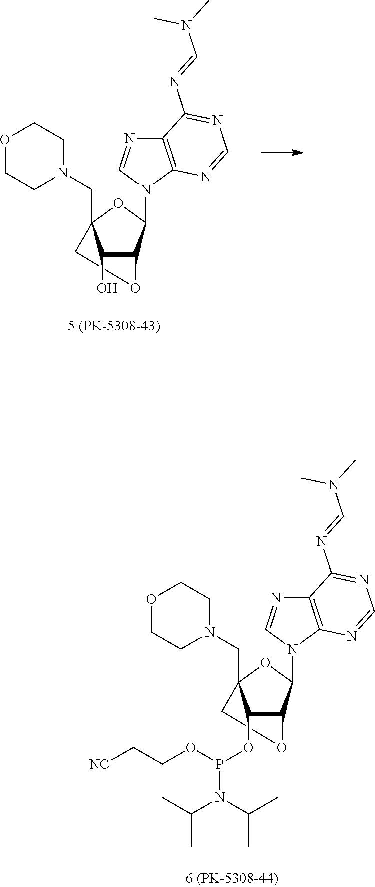 [SMILES: CN(C)/C=N/c1ncnc2c1ncn2[C@@H]1O[C@@]2(CN3CCOCC3)COC1[C@H]2O ; CC(C)N(C(C)C)P(OCCC#N)O[C@@H]1C2OC[C@]1(CN1CCOCC1)O[C@H]2n1cnc2c(/N=C/N(C)C)ncnc21]

### Synthesis of PK-5308-03

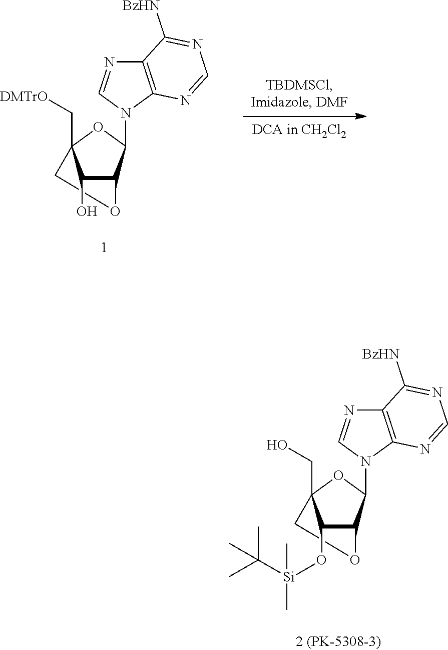

To a solution of nucleoside 1² (5.0 g, 7.29 mmol) in anhydrous DMF (20 mL) was added imidazole (0.74 g, 10.9 mmol), and tert-butyldimethylsilyl chloride (1.64 g, 10.9 mmol). The reaction mixture was stirred at room temperature for 20 h, diluted with EtOAc (200 mL) and washed with saturated aqueous solution of NaHCO₃ (2×100 mL). The combined aqueous phase was back extracted with EtOAc (100 mL). The combined organic phase was dried (MgSO₄) and concentrated under reduced pressure. To the residue was added a solution of dichloroacetic acid in CH₂Cl₂ (3% wt/v, 200 mL). The reaction mixture was stirred at room temperature for 1 h whereupon MeOH (5 mL was added). The reaction mixture was stirred again for 1 h. The reaction mixture was reduced to half under reduced pressure and neutralized with saturated aqueous solution of NaHCO₃ (200 mL) in an open flask. The content was transferred to a separating funnel and organic phase was washed with NaHCO₃ (200 mL). The combined aqueous phase was back extracted with CH₂Cl₂ (2×100 mL). The combined organic phase was dried (MgSO₄) and concentrated under reduced pressure. The residue was purified by column chromatography using a gradient of 0-2% MeOH in CH₂Cl₂ to afford 2 (PK-5308-03) (2.60 g, 72%). ¹H NMR (500 MHz, DMSO) δ 11.22 (s, 1H), 8.74 (s, 1H), 8.54 (s, 1H), 8.07-8.01 (m, 2H), 7.64 (t, J=7.4 Hz, 1H), 7.54 (t, J=7.7 Hz, 2H), 6.07 (s, 1H), 4.66 (s, 1H), 4.57 (s, 1H), 3.94 (d, J=7.8 Hz, 1H), 3.83 (d, J=7.8 Hz, 1H), 2.43 (d, J=7.2 Hz, 1H), 2.40 (d, J=7.1 Hz, 1H), 0.85 (s, 9H), 0.08 (s, 3H), 0.07 (s, 3H).

### Synthesis of nucleoside 3 (PK-5308-04)

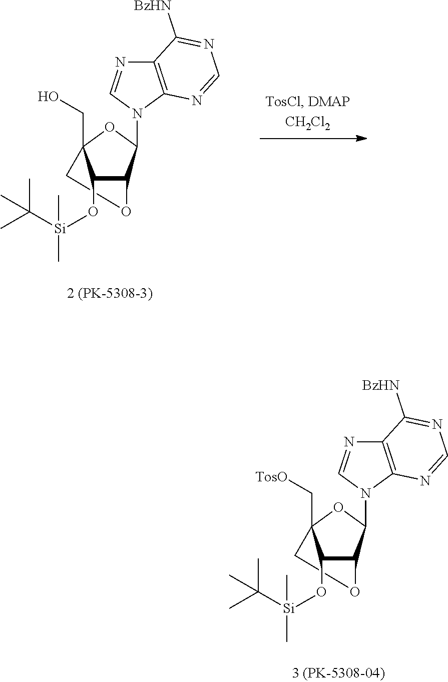

To a solution of nucleoside 2 (PK-5308-3) (2.60 g, 5.22 mmol) in dry CH₂Cl₂ was added 4-dimethylaminopyridine (DMAP, 1.27 g, 10.44 mmol) and 4-toluenesulfonyl chloride (TosCl, 1.24, 6.53 mmol) at 0° C. The reaction mixture was slowly warm to room temperature and stirred for 3 h. The reaction mixture was diluted with CH₂Cl₂ (100 mL) and washed with saturated aqueous solution of NaHCO₃ (50 mL). The aqueous phase was back extracted with CH₂Cl₂ (50 mL), and combined organic phase was dried (MgSO₄) and concentrated at reduced pressure. The residue was purified by column chromatography using a gradient of 0-5% MeOH in CH₂Cl₂ to afford nucleoside 3 (PK-5308-4) (2.65 g, 77%). ¹H NMR (400 MHz, DMSO) δ 11.25 (s, 1H), 8.73 (s, 1H), 8.48 (s, 1H), 8.12-7.96 (m, 2H), 7.76 (d, J=8.4 Hz, 2H), 7.66-7.62 (m, 1H), 7.57-7.53 (m, 2H), 7.47-7.40 (m, 2H), 6.07 (s, 1H), 4.74 (s, 1H), 4.72 (s, 1H), 4.59 (d, J=11.7 Hz, 1H), 4.23 (d, J=11.7 Hz, 1H), 3.84 (d, J=8.1 Hz, 1H), 3.79 (d, J=8.1 Hz, 1H), 2.38 (s, 3H), 0.78 (s, 9H), 0.07 (s, 3H), 0.05 (s, 3H). ¹³C NMR (126 MHz, DMSO) δ 165.55, 151.52, 151.31, 150.44, 145.31, 142.56, 133.26, 132.45, 131.46, 130.16, 128.45, 128.44, 127.68, 125.59, 85.61, 85.03, 78.88, 72.37, 71.31, 66.39, 25.34, 21.03, 17.43, −4.94, −5.37.

### Synthesis of nucleoside 4 (PK-5308-12)

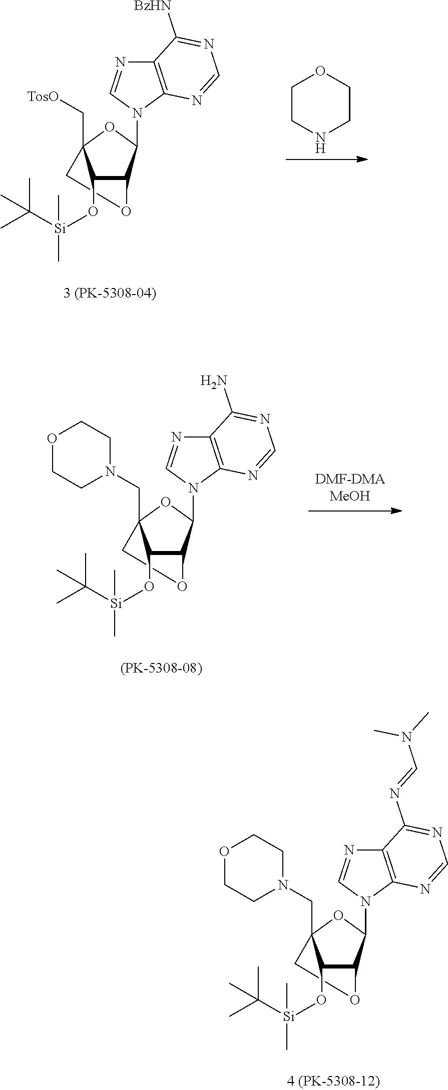

Nucleoside 3 (PK-5308-4) (2.5 g, 3.83 mmol) was taken in morpholine (30 mL). The reaction mixture for stirred at 60° C. for 2 days. The solvent was evaporated, and the crude was purified by column chromatography using a gradient of 0-8% MeOH in CH₂Cl₂ to afford PK-5308-08 (tentatively assigned by LCMS). To a solution of PK-5308-8 in MeOH (5 mL) was added DMF-DMA (400 μL, 3.01 mmol). The reaction mixture was stirred at room temperature for 18 h. Solvent was removed, and the residue was taken in CH₂Cl₂ (50 mL) and H₂O (50 mL). The layers were separated out and the aqueous phase was back extracted with CH₂Cl₂ (2×25 mL). The combined organic phase was dried (MgSO₄) and concentrated at reduced pressure. The residue was purified by column chromatography using a using a gradient of 0-5% MeOH in CH₂Cl₂ to afford 4 (PK-5308-12) (0.50 g, 25% from 3). ¹H NMR (400 MHz, DMSO) δ 8.90 (s, 1H), 8.40 (s, 1H), 8.29 (s, 1H), 5.98 (s, 1H), 4.70 (s, 1H), 4.55 (s, 1H), 3.92 (d, J=8.0 Hz, 1H), 3.86 (d, J=8.0 Hz, 1H), 3.53 (t, J=4.8 Hz, 4H), 3.19 (s, 3H), 3.12 (s, 3H), 2.74 (d, J=14.2 Hz, 1H), 2.69 (d, J=14.2 Hz, 1H), 2.57-2.50 (m, 2H), 2.45-2.36 (m, 2H), 0.85 (s, 9H), 0.09 (s, 3H), 0.08 (s, 3H). ¹³C NMR (126 MHz, DMSO) δ 159.25, 157.97, 151.83, 150.48, 140.75, 125.58, 88.06, 85.53, 78.73, 73.03, 72.30, 66.17, 55.06, 54.73, 40.67, 34.56, 25.52, 17.64, −4.83, −5.14.

### Synthesis of nucleoside 5 (PK-5308-43)

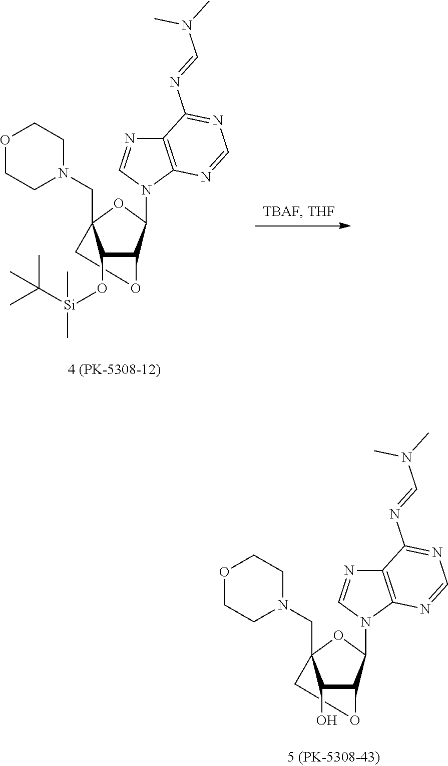 [SMILES: CN(C)/C=N/c1ncnc2c1ncn2[C@@H]1O[C@@]2(CN3CCOCC3)COC1[C@H]2O[Si](C)(C)C(C)(C)C ; CN(C)/C=N/c1ncnc2c1ncn2[C@@H]1O[C@@]2(CN3CCOCC3)COC1[C@H]2O]

Nucleoside 4 (PK-5308-12) (1.0 g, 1.93 mmol) was dissolved in THF (5 mL). To this was added TBAF (1M in THF, 2.4 mL, 2.4 mmol), and the reaction mixture was stirred at room temperature for 1 h. Solvent was removed, and the residue was purified by column chromatography using a using a gradient of 0-6% MeOH in EtOAc to afford 5 (PK-5308-43) (0.58 g, 74%). ¹H NMR (500 MHz, DMSO) δ 8.90 (s, 1H), 8.41 (s, 1H), 8.31 (s, 1H), 5.95 (s, 1H), 5.78 (d, J=4.4 Hz, 1H), 4.41 (s, 1H), 4.26 (d, J=4.4 Hz, 1H), 3.95 (d, J=8.1 Hz, 1H), 3.85 (d, J=8.1 Hz, 1H), 3.56 (t, J=4.8 Hz, 4H), 3.19 (s, 3H), 3.12 (s, 3H), 2.82 (d, J=14.3 Hz, 1H), 2.77 (d, J=14.3 Hz, 1H), 2.66-2.57 (m, 2H), 2.46-2.35 (m, 2H). ¹³C NMR (126 MHz, DMSO) δ 159.16, 157.95, 152.01, 150.47, 139.72, 125.51, 88.06, 85.24, 78.89, 71.94, 71.38, 66.21, 55.26, 54.66, 40.64, 34.53.

### Synthesis of nucleoside 6 (PK-5308-44)

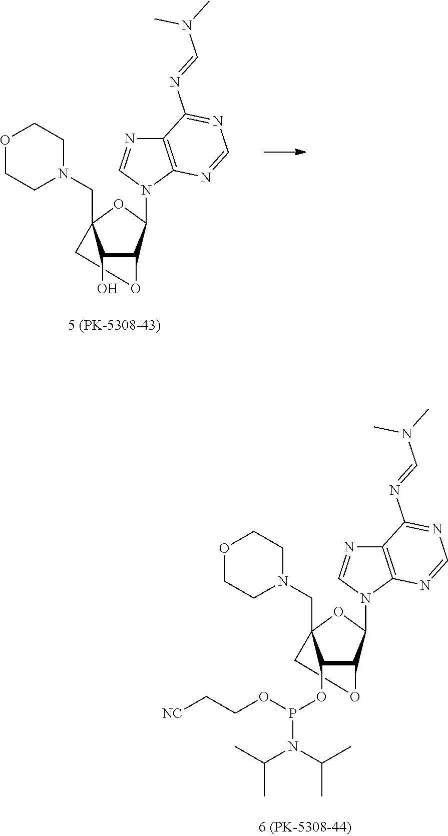 [SMILES: CN(C)/C=N/c1ncnc2c1ncn2[C@@H]1O[C@@]2(CN3CCOCC3)COC1[C@H]2O ; CC(C)N(C(C)C)P(OCCC#N)O[C@@H]1C2OC[C@]1(CN1CCOCC1)O[C@H]2n1cnc2c(/N=C/N(C)C)ncnc21]

To a suspension of nucleoside 5 (PK-5308-43) (0.55 g, 1.36 mmol) in anhydrous CH₂Cl₂ (5 mL) was added 2-Cyanoethyl N, N,N′, N′-tetraisopropylphosphordiamidite (PN₂ 0.82 g, 2.72 mmol) followed by 4,5 dicyanoimidazole (DCI, 0.20 mg, 1.7 mmol). The reaction mixture was stirred at room temperature for 3 h whereupon it was diluted with CH₂Cl₂ (50 mL) and washed with saturated aqueous NaHCO₃ (25 mL). The aqueous phase was back extracted with CH₂Cl₂ (20 mL), and the combined organic phase was dried (MgSO₄) and concentrated at reduced pressure. The residue was purified by column chromatography using a gradient of 0-2% MeOH in CH₂Cl₂ (containing 0.2% Et₃N) to afford 6 (PK-5308-44) (0.65 g, 79%). ³¹P NMR (202 MHz, CD₃CN) δ 149.80, 149.71.

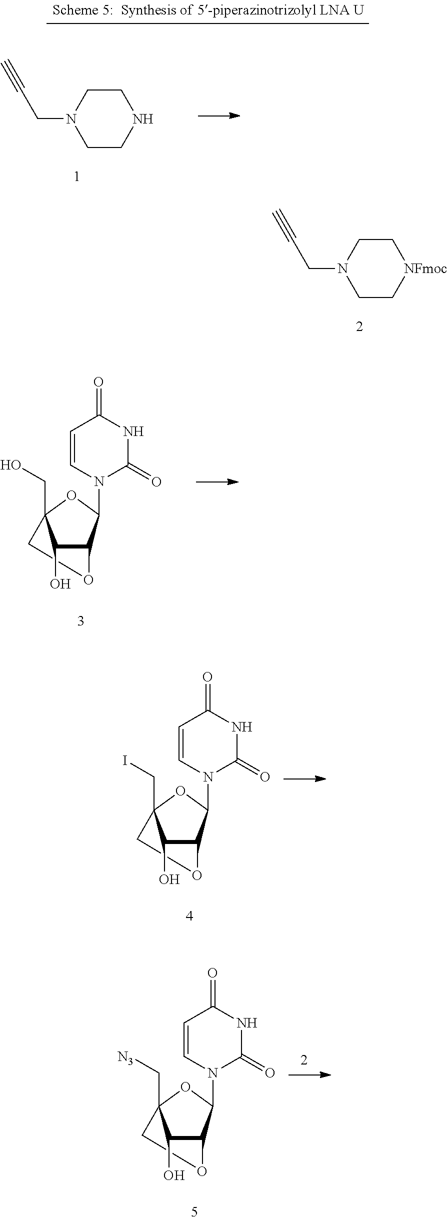

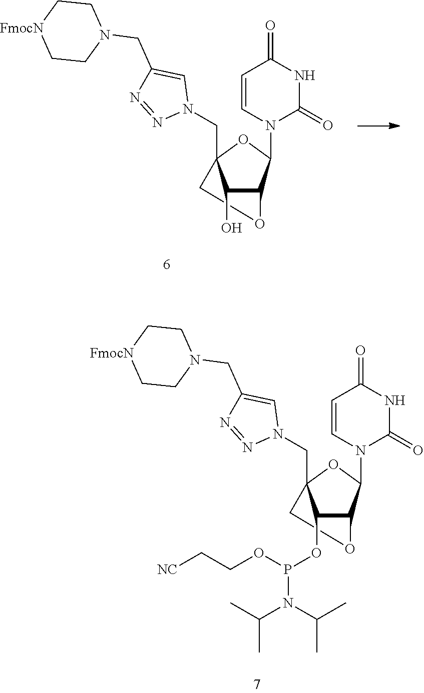

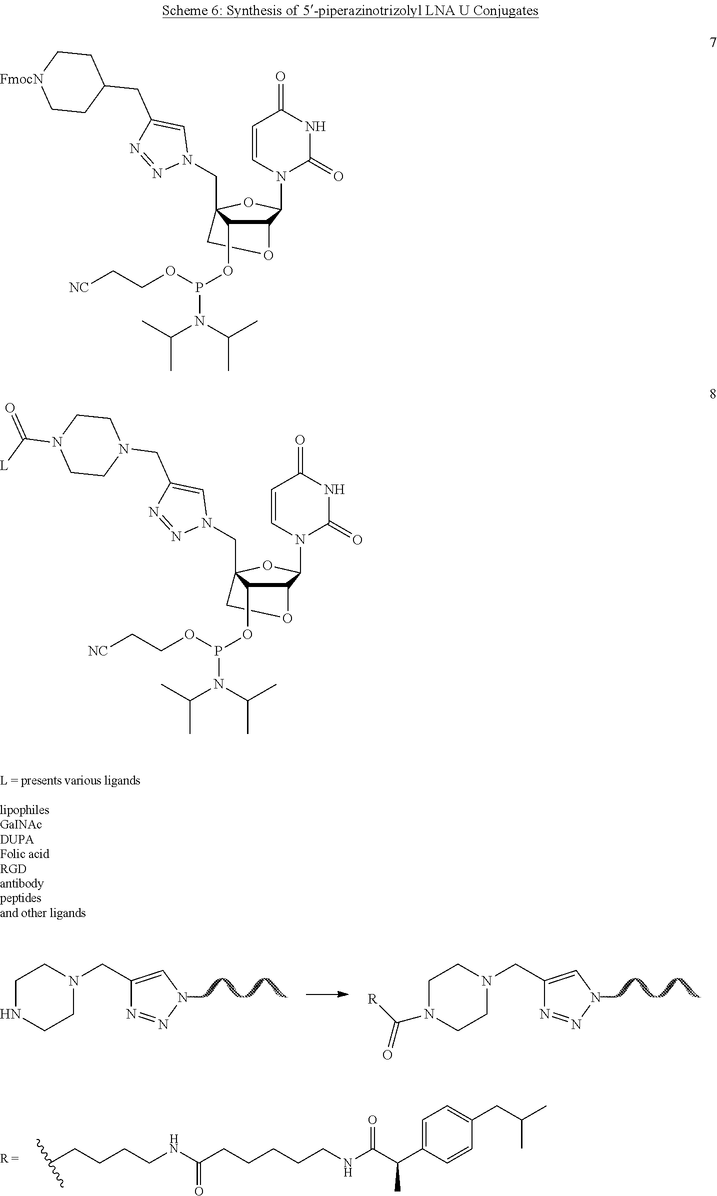

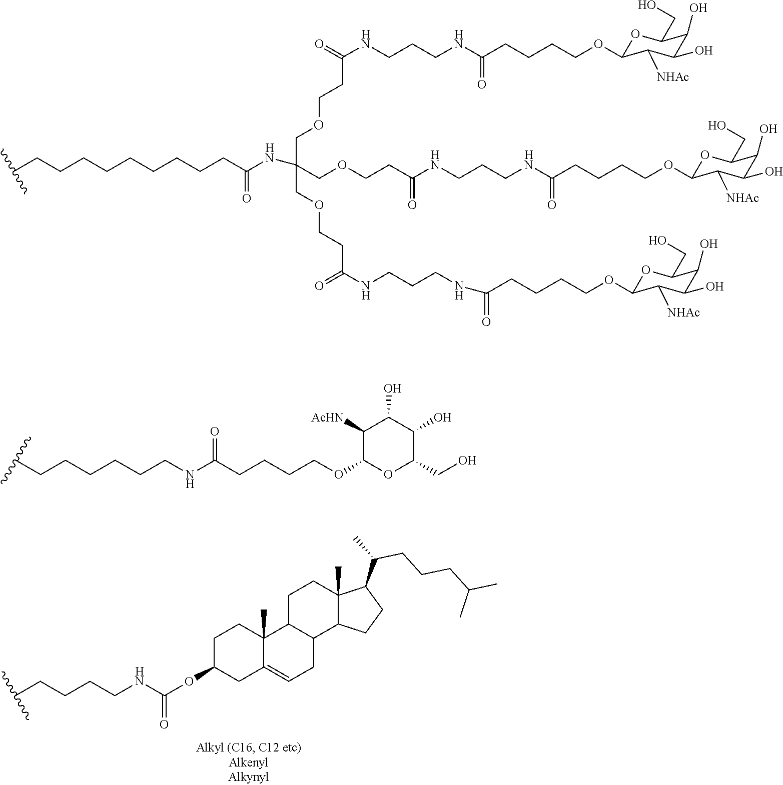 [SMILES: *CCCCCCCCCC(=O)NC(COCCC(=O)NCCCNC(=O)CCCCOC1OC(CO)C(O)C(O)C1NC(C)=O)(COCCC(=O)NCCCNC(=O)CCCCOC1OC(CO)C(O)C(O)C1NC(C)=O)COCCC(=O)NCCCNC(=O)CCCCOC1OC(CO)C(O)C(O)C1NC(C)=O ; *CCCCCCNC(=O)CCCCO[C@H]1O[C@@H](CO)[C@@H](O)[C@@H](O)[C@@H]1NC(C)=O ; *CCCCNC(=O)O[C@H]1CC[C@@]2(C)C(=CCC3C2CC[C@@]2(C)C3CC[C@@H]2[C@H](C)CCCC(C)C)C1]

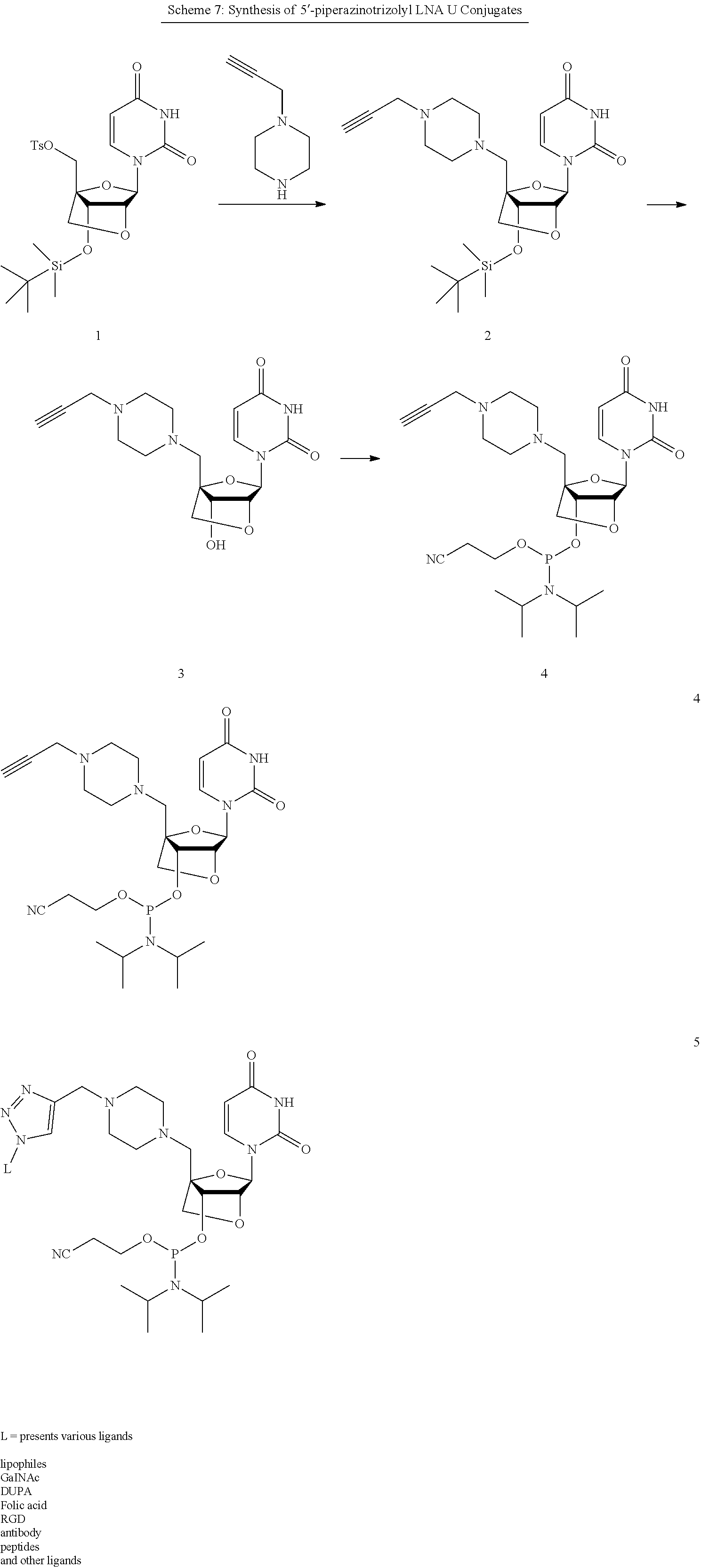

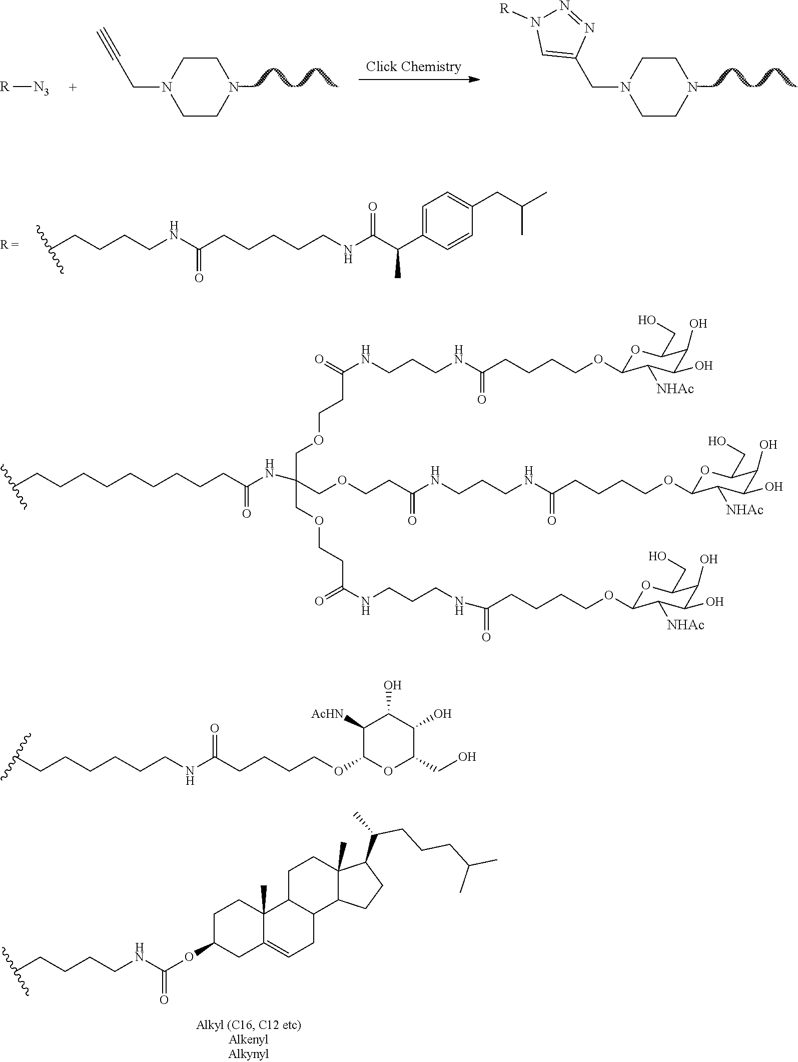

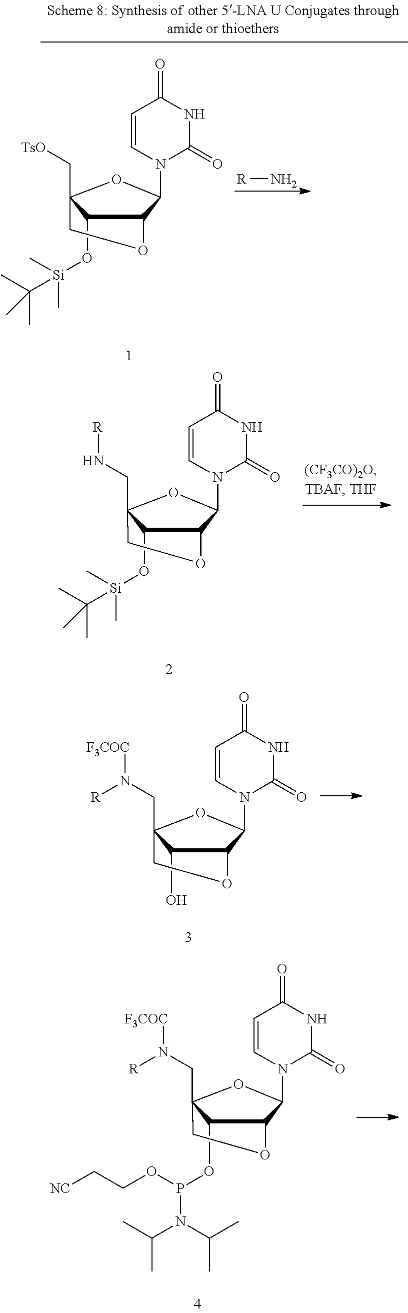

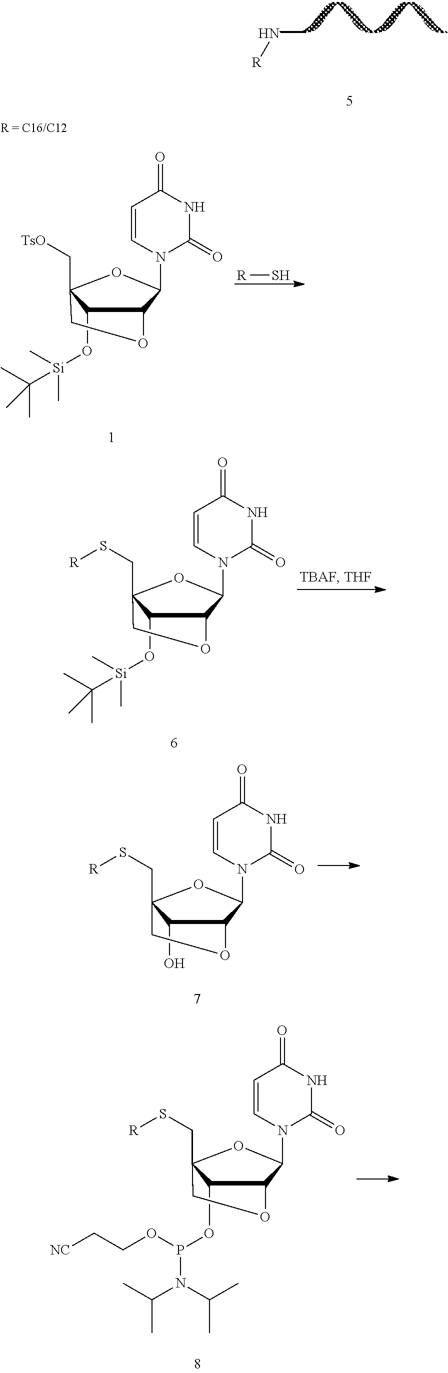

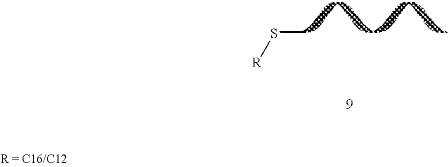

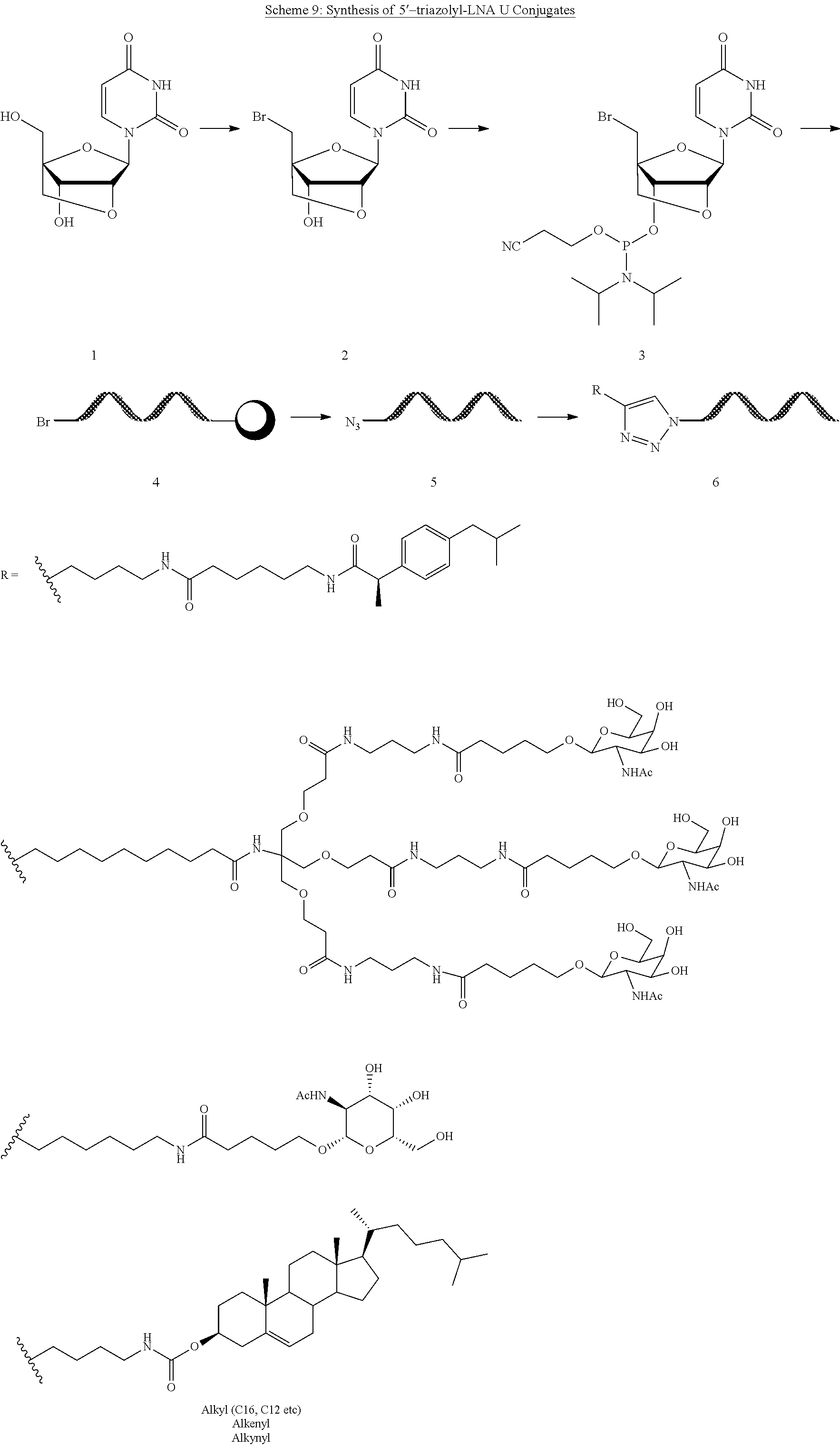

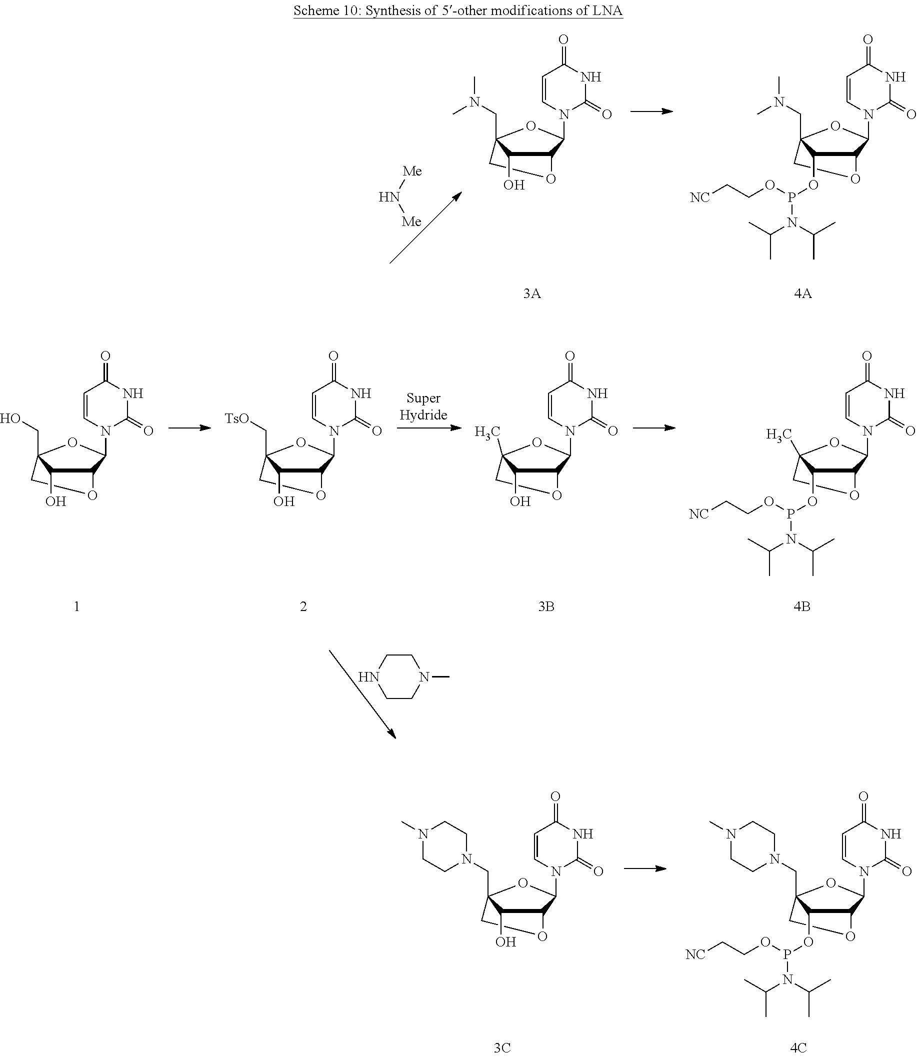

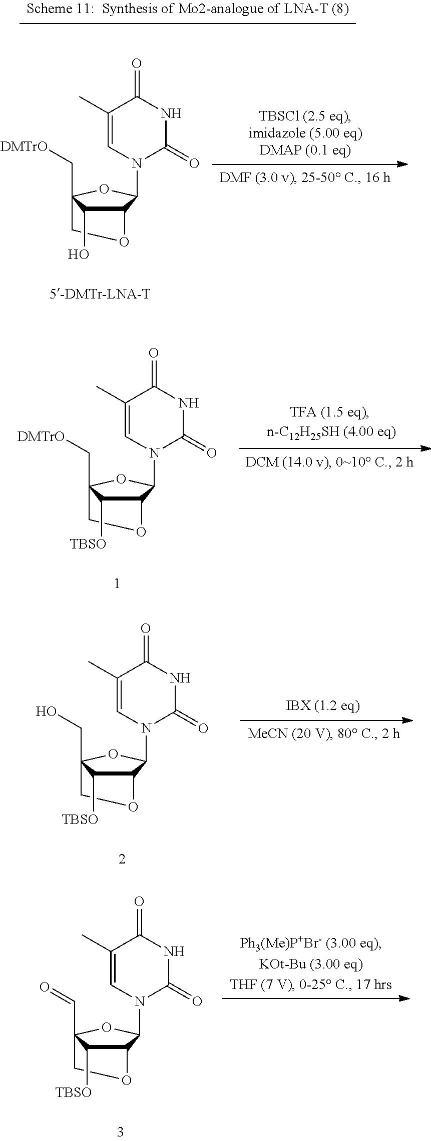

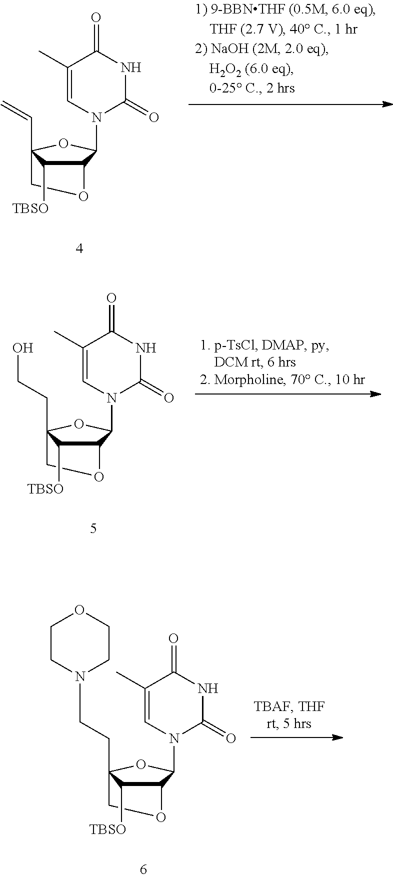 [SMILES: C=C[C@]12CO[C@@H](C1O[Si](C)(C)C(C)(C)C)[C@H](n1cc(C)c(=O)[nH]c1=O)O2 ; Cc1cn([C@@H]2O[C@@]3(CCO)CO[C@H]2C3O[Si](C)(C)C(C)(C)C)c(=O)[nH]c1=O ; Cc1cn([C@@H]2O[C@@]3(CCN4CCOCC4)CO[C@H]2C3O[Si](C)(C)C(C)(C)C)c(=O)[nH]c1=O]

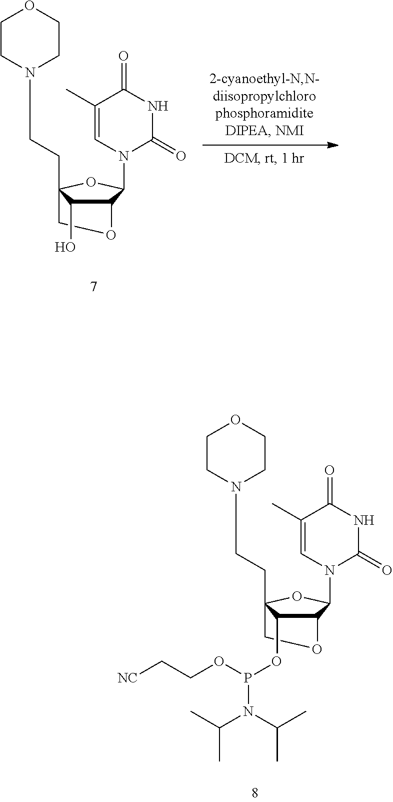 [SMILES: Cc1cn([C@@H]2O[C@@]3(CCN4CCOCC4)CO[C@H]2C3O)c(=O)[nH]c1=O ; Cc1cn([C@@H]2O[C@@]3(CCN4CCOCC4)CO[C@H]2C3OP(OCCC#N)N(C(C)C)C(C)C)c(=O)[nH]c1=O]

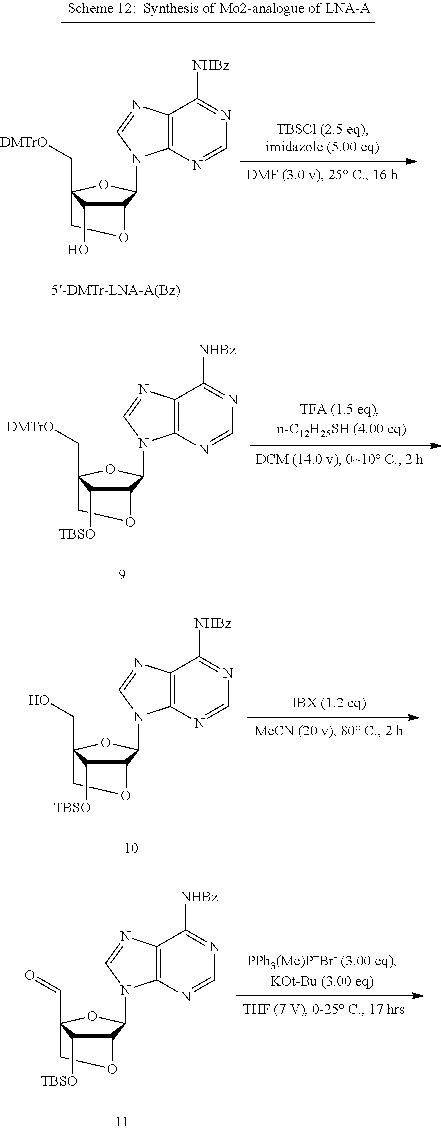

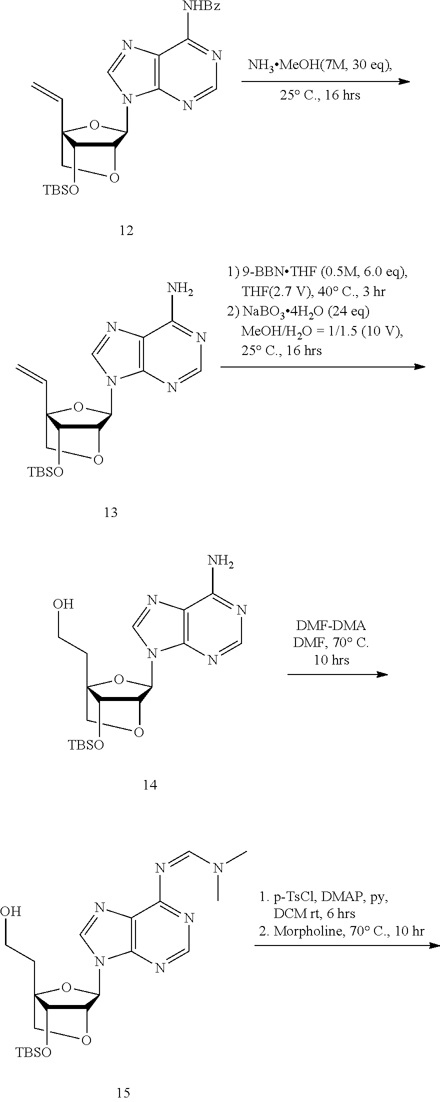

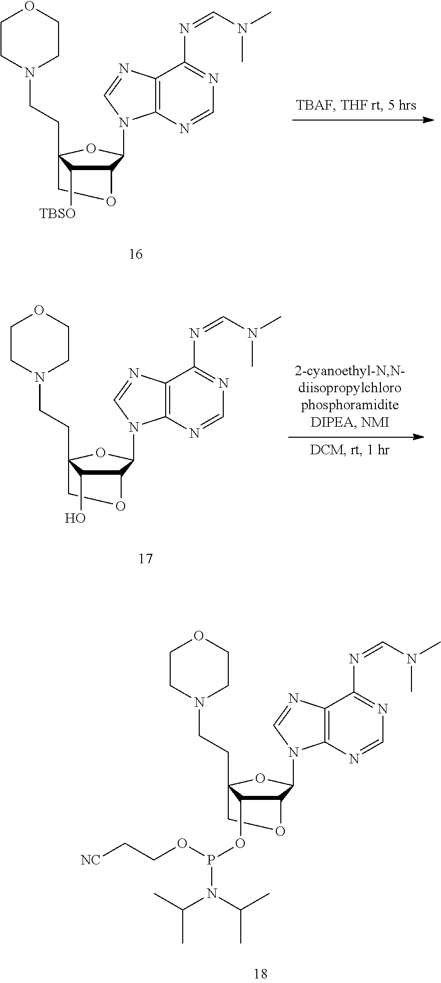 [SMILES: CN(C)/C=N\c1ncnc2c1ncn2[C@@H]1O[C@@]2(CCN3CCOCC3)CO[C@H]1C2O[Si](C)(C)C(C)(C)C ; CN(C)/C=N\c1ncnc2c1ncn2[C@@H]1O[C@@]2(CCN3CCOCC3)CO[C@H]1C2O ; CC(C)N(C(C)C)P(OCCC#N)OC1[C@@H]2OC[C@]1(CCN1CCOCC1)O[C@H]2n1cnc2c(/N=C\N(C)C)ncnc21]

### Oligonucleotide Synthesis

### Codex Monomers:

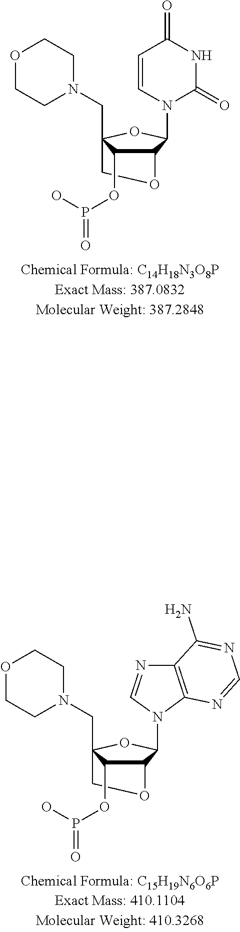

Oligonucleotide synthesis was carried out on ABI using standard procedure. The synthesis was performed at 1 μM scale using 5-ethylthio-1H-tetrazole as activator (0.25 M in dry CH₃CN), and I₂ in THF: pyridine as an oxidizer (for PO linkages). For PS linkages DDTT was used as an oxidizer. No final detritylation was performed for strands containing 5′-morpholino monomers.

The cleaveage and deprotection was performed using methylamine solution at room temperature for 90 minutes. And purification was performed using IEX buffers with a gradient of 15-40% buffer B over 30 column volume. Buffer A: 20 mM sodium phosphate contains 10-15% CH₃CN, Buffer B: 1M NaBr, 20 mM sodium phosphate contains 10-15% CH₃CN. After purification fractions containing pure oligonucleotide were pooled and desalted using sephadex columns.

TABLE 14
Exemplary modified oligonucleotides synthesized

| | | ON | SEQ | Mass | Mass |
|---|---|---|---|---|---|
| | | sequence | ID | ob- | calcu- |
| # | ALNY# | 5′-3′ | NO. | tained | lated |
| 1 | A- | Q340sasCfaGfuGfu | 207 | 8667.80 | 8669.29 |
| | 1036881.1 | UfCfUfuGfcUfcUfa | | | |
| | | UfaAfL96 | | | |
| 2 | A- | Q340asCfaGfuGfuU | 208 | 8651.70 | 8653.23 |
| | 1036882.1 | fCfUfuGfcUfcUfaU | | | |
| | | faAfL96 | | | |
| 3 | A- | Q340aCfaGfuGfuUf | 209 | 8635.56 | 8637.16 |
| | 1036883.1 | CfUfuGfcUfcUfaUf | | | |
| | | aAfL96 | | | |
| 4 | A- | Q340saCfaGfuGfuU | 210 | 8651.56 | 8653.23 |
| | 1037070.1 | fCfUfuGfcUfcUfaU | | | |
| | | faAfL96 | | | |
| 5 | A- | Q339sgsGfaAfgCfa | 211 | 8866.65 | 8867.44 |
| | 1037055.1 | GfUfAfuGfuUfgAfu | | | |
| | | GfgAfL96 | | | |
| 6 | A- | Q339gsGfaAfgCfaG | 212 | 8850.55 | 8851.38 |
| | 1037056.1 | fUfAfuGfuUfgAfuG | | | |
| | | fgAfL96 | | | |
| 7 | A- | Q339gGfaAfgCfaGf | 213 | 8834.67 | 8835.31 |
| | 1037057.2 | UfAfuGfuUfgAfuGf | | | |
| | | gAfL96 | | | |
| 8 | A- | Q339sgGfaAfgCfaG | 214 | 8850.54 | 8851.38 |
| | 1038565.1 | fUfAfuGfuUfgAfuG | | | |
| | | fgAfL96 | | | |
| 9 | A- | Q339sgsUfgAfcAfa | 215 | 8793.80 | 8794.43 |
| | 1036884.1 | AfUfAfuGfgGfcAfu | | | |
| | | CfaAfL96 | | | |
| 10 | A- | Q339gsUfgAfcAfaA | 216 | 8777.61 | 8778.37 |
| | 1036885.1 | fUfAfuGfgGfcAfuC | | | |
| | | faAfL96 | | | |
| 11 | A- | Q339gUfgAfcAfaAf | 217 | 8761.71 | 8762.30 |
| | 1036886.1 | UfAfuGfgGfcAfuCf | | | |
| | | aAfL96 | | | |
| 12 | A- | Q339sgUfgAfcAfaA | 218 | 8777.53 | 8778.37 |
| | 1308564.1 | fUfAfuGfgGfcAfuC | | | |
| | | faAfL96 | | | |
| 13 | A- | (Alns)asCfaGfuGf | 219 | 8599.50 | 8600.18 |
| | 265443.3 | uUfCfUfuGfcUfcUf | | | |
| | | aUfaAfL96 | | | |
| 14 | A- | (Aln)asCfaGfuGfu | 220 | 8583.40 | 8584.12 |
| | 899883.2 | UfCfUfuGfcUfcUfa | | | |
| | | UfaAfL96 | | | |
| 15 | A- | (Aln)aCfaGfuGfuU | 221 | 8567.30 | 8568.05 |
| | 899884.2 | fCfUfuGfcUfcUfaU | | | |
| | | faAfL96 | | | |
| 16 | A- | (Alns)aCfaGfuGfu | 222 | 8583.39 | 8584.12 |
| | 1318094.1 | UfCfUfuGfcUfcUfa | | | |
| | | UfaAfL96 | | | |

Structures of Q339, Q340, (Aln) and (Alns) are shown in FIG. 33.

dsRNAs

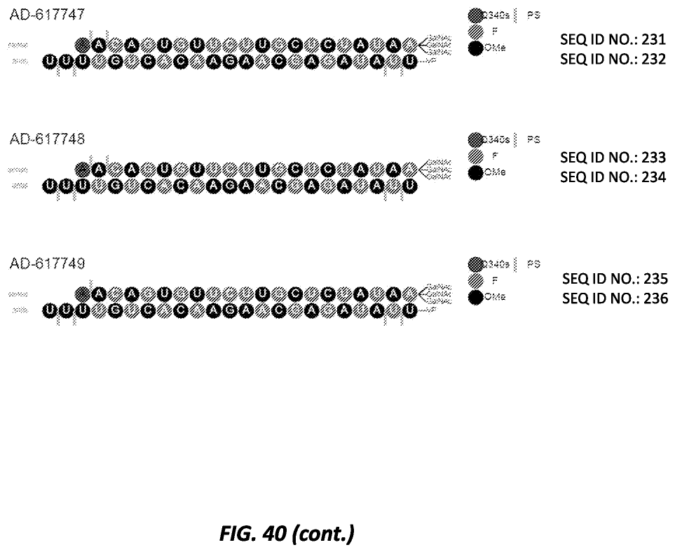

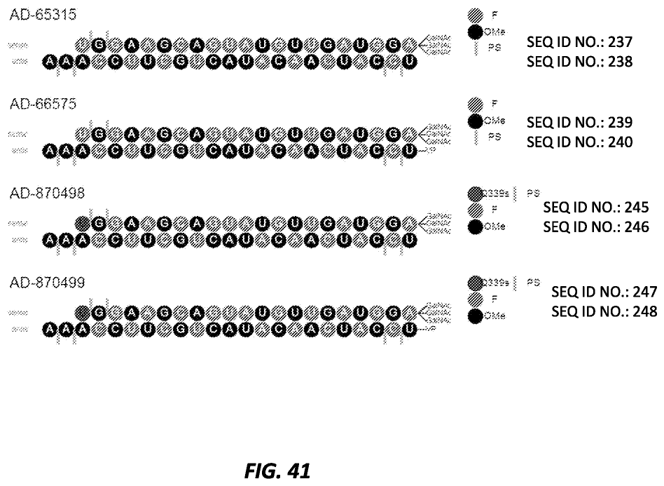

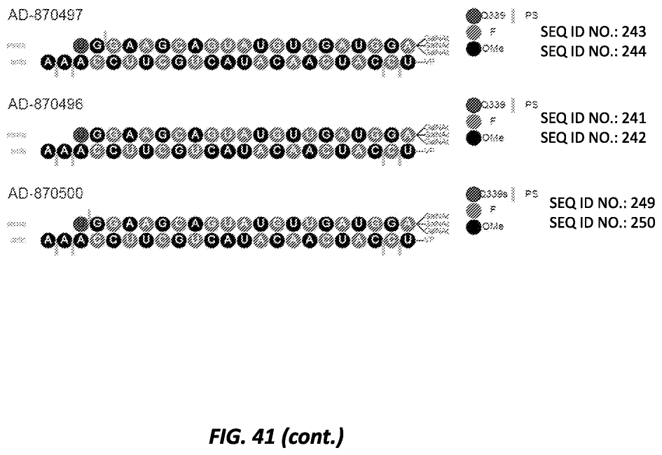

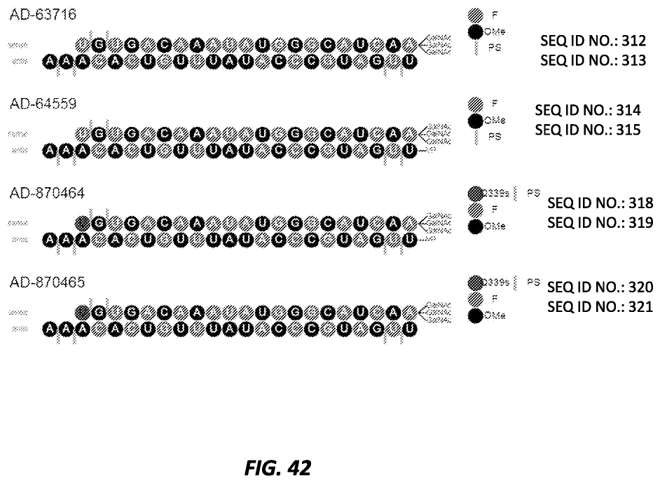

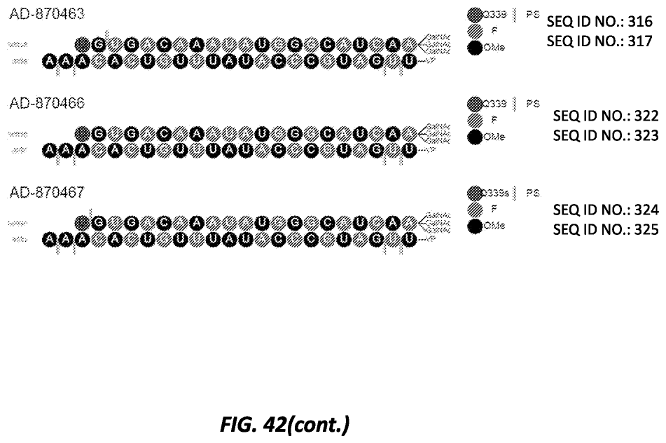

The modified strands were then mixed with appropriate antisense strands (obtained from inventory) and annealed together in 1×PBS buffer to obtain the exemplary duplexes shown in FIGS. 40-43.

### In Vitro Gene Silencing

5 μL of siRNA was placed in a 384-well collagen-coated plate. To each well was added 5.0 μL of Opti-MEM and 0.1 μL of Lipofectamine RNAiMax (Invitrogen). The final siRNA concentrations were 0.1 or 10 nM. Plates were incubated at room temperature for 15 min. Primary mouse hepatocytes cells were suspended in media Invitrogro CP rodent medium (#Z990028 BioIVT) and 40 μL of this suspension (containing approximately 5000 cells) was added to each well. Each siRNA was assessed in quadruplicate. After incubating the cells for 24 h, RNA was isolated using DynaBeads (ThermoFisher). The RNA was then reverse transcribed into cDNA according to manufacturer's protocol (Applied Biosystems). Multiplex qPCR reactions were performed in duplicate using a gene specific TaqMan assay for Ttr (ThermoFisher Scientific, #Mm00443267_m1) and mouse Gapdh (#4352339E) as an endogenous control. Real-Time PCR was performed on a Roche LightCycler 480 using LightCycler 480 Probes Master Mix (Roche).

### In Vivo Gene Silencing

COS-7 cells were cultured at 37° C., 5% CO₂ in Dulbecco's Modified Eagle Medium (DMEM) supplemented with 10% fetal bovine serum (FBS). Cells were co-transfected in 96-well plates (15,000 cells/well) with 10 ng luciferase reporter plasmid and 0.64 pM to 50 nM siRNA in 5-fold dilutions using 2 μg/mL Lipofectamine 2000 (Thermo Fisher Scientific) according to manufacturer's instructions. Cells were harvested at 48 h after transfection for the dual luciferase assay (Promega) according to manufacturer's instructions. The on-target reporter plasmid contained a single perfectly-complementary site to the antisense strand in the 3′ untranslated (3′ UTR) of Renilla luciferase. The off-target reporter plasmid contained four tandem seed-complementary sites separated by a 19-nucleotide spacer d (TAATATTACATAAATAAAA) (SEQ ID NO. 259) in the 3′ UTR of Renilla luciferase. Both plasmids co-expressed Firefly luciferase as a transfection control.

### In Vivo Metabolic Stability

Female, 6-8 weeks old C56BL/6 mice were dosed at 1 mg/kg subcutaneously, with the control group receiving same volume of 1×PBS. Each group comprises 3 mice. Blood samples were collected just prior to treatment administration, and 3, 7, 14, 21 and 28 days after dosing. The collected serum samples were stored in −80° C. until further analysis. TTR protein levels in serum were quantified spectrophotometrically with a mouse pre-albumin/TTR ELISA kit (41-PALMS-E01), in accordance to the manufacturer's protocol (ALPCO, Salem, NH).

At day 28, the animals were euthanized, with the livers harvested and then cryopreserved. RNA from liver was isolated using the PerkinElmer Chemagic system (Waltham, MA), according to the supplier's guidelines. This was followed by cDNA preparation and multiplexed RT-qPCR analysis to assess the TTR transcript levels (Taqman probe Mm00443267_ml; mouse Gapdh 4351309 from ABI).

Schemes 4 and 5 show exemplary methods for the synthesis of modified phosphoramidites. Accordingly, 3′-O-TBDMS nucleotides were prepared in a single step from commercially available nucleosides. Tosylation followed by heating with morpholine produced 5′-morpholino nucleoside. The loss of benzoyl group was observed during the reaction with morpholine. The amidine group was installed. Removal of 3′-OTBDMS group followed by phosphitylation gave required amidites.

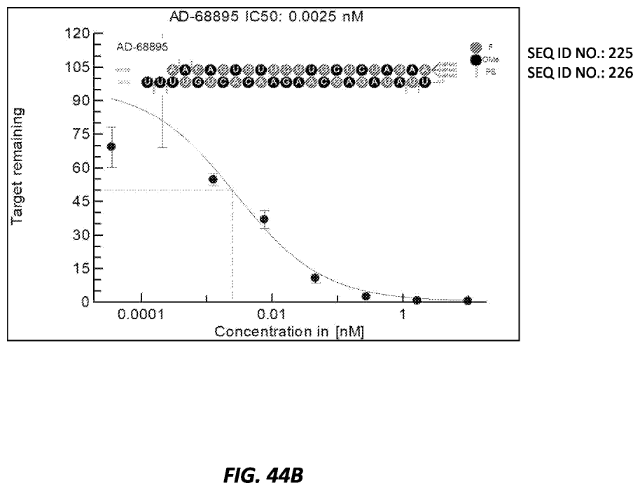

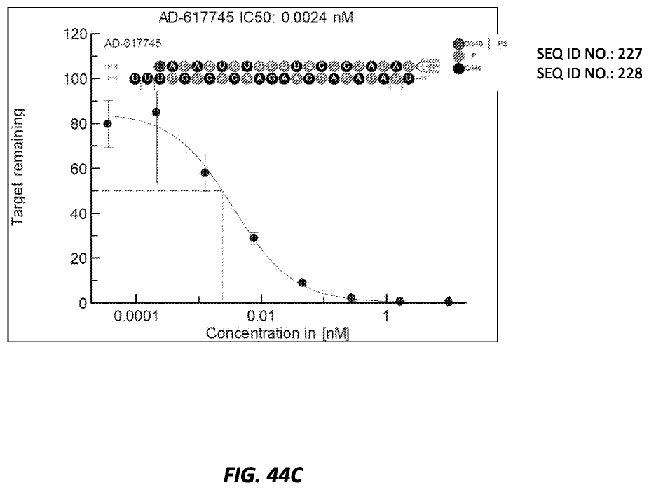

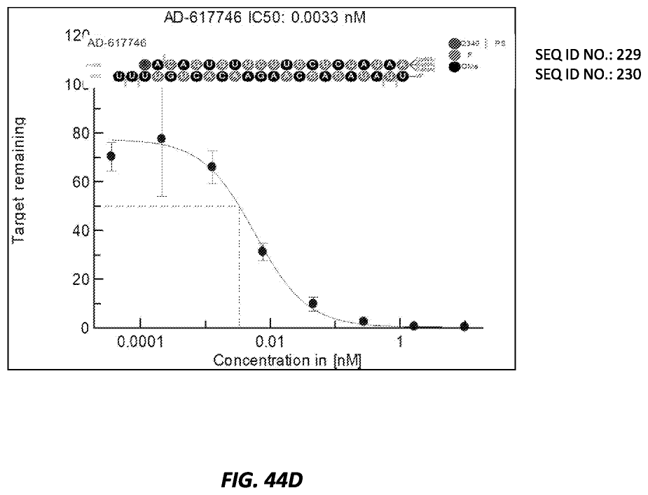

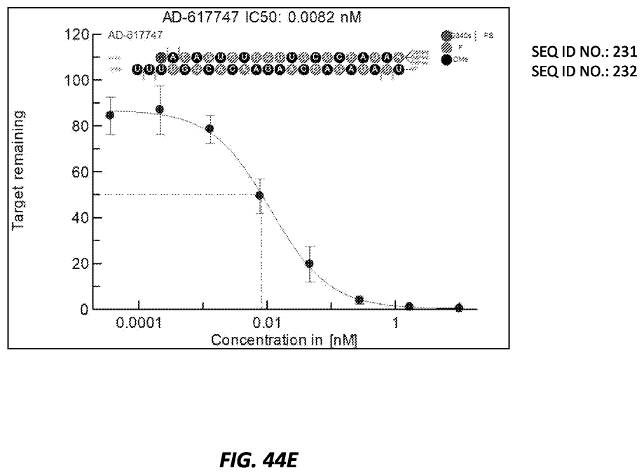

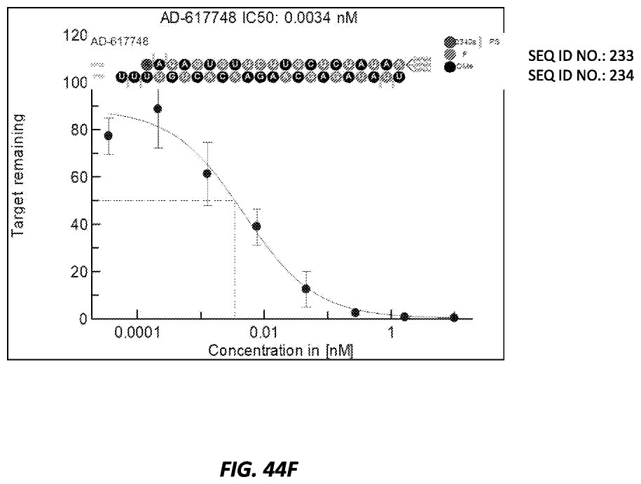

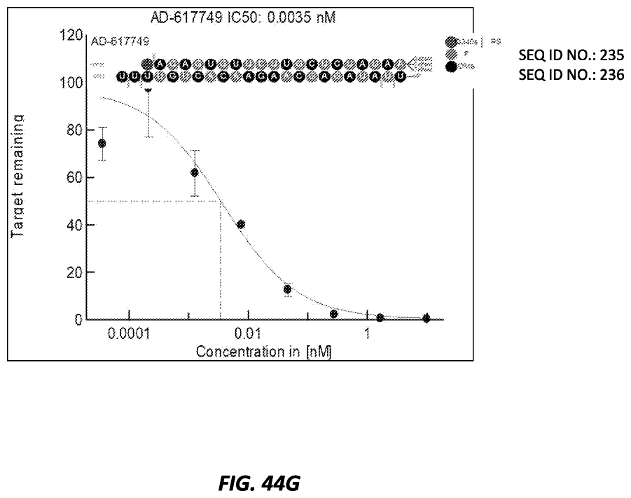

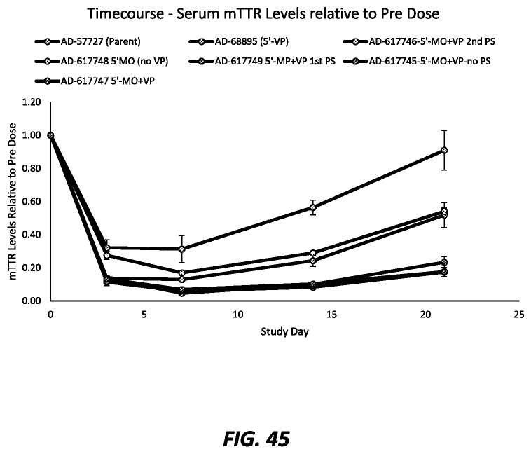

In vitro screening: To gauge the impact of combination of 5′-VP (antisense strand) and 5′-morpholino (sense strand), we chose our well-studied GalNAc-conjugated parent siRNA duplex targeting TTR. The study involved a set of 7 duplexes namely parent duplex (without any modification at the 5′-ends, entry 1), a duplex that carry 5′-VP on the antisense strand (entry 2), a duplex that carry 5′-morpholino on the sense strand (entry 3) and a duplex that carry both 5′-VP and 5′-morpholino modification (entry 4). Other three duplexes (entry 5-7) differ from duplex 4 only in the content of PS linkages in the sense strand. Results are shown in FIGS. 44A-44G and summarized in Table 15.

TABLE 15
IC₅₀ data on the on-target activity in
luciferase reporter assay for duplexes
that target mTTR

| | | | SEQ | | |
|---|---|---|---|---|---|
| | Alny | | ID | IC5 | |
| # | # | Duplex | No. | 0(nM) | |
| 1 | 57727 | 5′-AfsasCfaGfuGfuUfCfUfuGfcUfcUfaUfaAfL96 | 223 | 0.004 | Parent |
| | | 5′-usUfsaUfaGfaGfcAfagaAfcAfcUfgUfususu | 224 | | |
| 2 | 68895 | 5′-AfsasCfaGfuGfuUfCfUfuGfcUfcUfaUfaAfL96 | 225 | 0.0025 | Sense-OH |
| | | 5′-VPusUfsaUfaGfaGfcAfagaAfcAfcUfgUfususu | 226 | | Antisense-VP |
| 3 | 617745 | 5′-Q340aCfaGfuGfuUfCfUfuGfcUfcUfaUfaAfL96 | 227 | 0.0024 | Sense-Mo-no PS |
| | | 5′-VPusUfsaUfaGfaGfcAfagaAfcAfcUfgUfususu | 228 | | Antisense-VP |
| 4 | 617746 | 5′-Q340asCfaGfuGfuUfCfUfuGfcUfcUfaUfaAfL96 | 229 | 0.0033 | Sense-Mo-2^{nd} PS |
| | | 5′-VPusUfsaUfaGfaGfcAfagaAfcAfcUfgUfususu | 230 | | Antisense-VP |
| 5 | 617747 | 5′-Q340sasCfaGfuGfuUfCfUfuGfcUfcUfaUfaAfL96 | 231 | 0.0082 | Sense-Mo |
| | | 5′-VPusUfsaUfaGfaGfcAfagaAfcAfcUfgUfususu | 232 | | Antisense-VP |
| 6 | 617748 | 5′-Q340sasCfaGfuGfuUfCfUfuGfcUfcUfaUfaAfL96 | 233 | 0.0034 | Sense-Mo |
| | | 5′-usUfsaUfaGfaGfcAfagaAfcAfcUfgUfususu | 234 | | Antisense-OH |
| 7 | 617749 | 5′-Q340saCfaGfuGfuUfCfUfuGfcUfcUfaUfaAfL96 | 235 | 0.0035 | Sense-Mo-1st PS |
| | | 5′-VPusUfsaUfaGfaGfcAfagaAfcAfcUfgUfususu | 236 | | Antisense-VP |

Structure of Q340 is shown in FIG. 33.

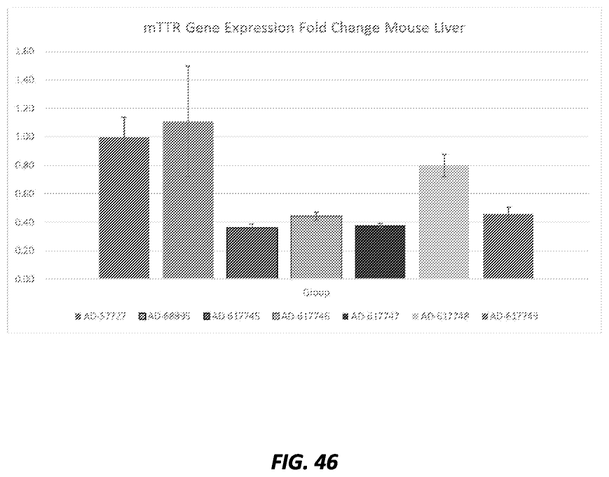

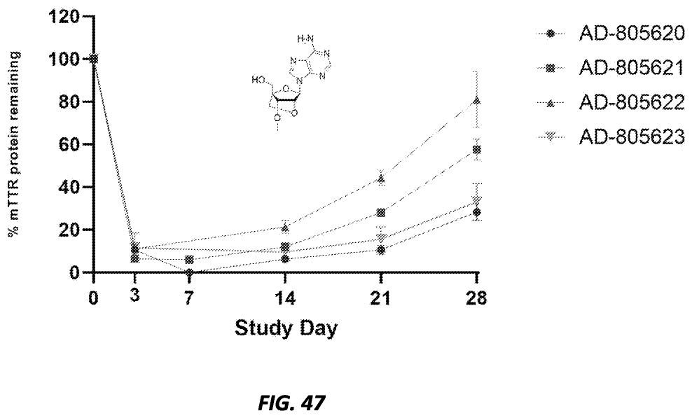

Comparison of duplex 1 and 2 show the impact of VP on the RNAi activity (FIGS. 44A and 44B). As shown before presence of VP improves the efficacy. Duplexes 1 and 3 show comparable efficacy meaning presence of morpholino have no negative impact on activity (FIGS. 44A and 44C). Duplex 2, 4, 6, and 7 also showed similar activity suggesting no impact of absence or presence of PS backbone (FIGS. 44B, 44D, 44F and 44G). However, at day 7 showed significant improvement in efficacy for the duplexes carrying 5′-morpholino group than the corresponding duplexes with 5′-OH on the sense strand (FIGS. 44A-44G). Furthermore, activity was not affected by absence of PS backbone indicating that the LNA-morpholino can be providing enough metabolic stability even in the absence of PS backbone. Similar conclusion was made from day 28 qPCR data (FIG. 46). Thus, combination of VP-Morpholino showed the best activity and may not require presence of PS backbone on the sense strand. Reducing PS content would make the drug candidate stereochemically purer.

TABLE 16
IC₅₀ data on the on-target activity in
luciferase reporter assay for duplexes
that target F9

| Du- | | | SEQ | |
|---|---|---|---|---|
| plex | Alny | | ID | IC50 |
| # | # | Duplex | NO. | (nM) |
| 1 | 65315 | 5′-UfsgsGfaAfgCfaGfUf | 237 | 1.6511 |
| | | AfuGfuUfgAfuGfgAfL96 | | |
| | | 5′-usCfscAfuCfaAfcAfu | 238 | |
| | | acUfgCfuUfcCfasasa | | |
| 2 | 66575 | 5′-UfsgsGfaAfgCfaGfUf | 239 | 0.2849 |
| | | AfuGfuUfgAfuGfgAfL96 | | |
| | | 5′-VPusCfscAfuCfaAfcA | 240 | |
| | | fuacUfgCfuUfcCfasasa | | |
| 3 | 870496 | 5′-Q339gGfaAfgCfaGfUf | 241 | 0.2795 |
| | | AfuGfuUfgAfuGfgAfL96 | | |
| | | 5′-VPusCfscAfuCfaAfcA | 242 | |
| | | fuacUfgCfuUfcCfasasa | | |
| 4 | 870497 | 5′-Q339gsGfaAfgCfaGfU | 243 | 0.3168 |
| | | fAfuGfuUfgAfuGfgAfL96 | | |
| | | 5′-VPusCfscAfuCfaAfcA | 244 | |
| | | fuacUfgCfuUfcCfasasa | | |
| 5 | 870498 | 5′-Q339sgsGfaAfgCfaGf | 245 | 1.6 |
| | | UfAfuGfuUfgAfuGfgAfL | | |
| | | 96 | | |
| | | 5′-usCfscAfuCfaAfcAfu | 246 | |
| | | acUfgCfuUfcCfasasa | | |
| 6 | 870499 | 5′-Q339sgsGfaAfgCfaGf | 247 | 0.163 |
| | | UfAfuGfuUfgAfuGfgAfL | | |
| | | 96 | | |
| | | 5′-VPusCfscAfuCfaAfcA | 248 | |
| | | fuacUfgCfuUfcCfasasa | | |
| 7 | 870500 | 5′-Q339sgGfaAfgCfaGfU | 249 | 0.086 |
| | | fAfuGfuUfgAfuGfgAfL96 | | |
| | | 5′-VPusCfscAfuCfaAfcA | 250 | |
| | | fuacUfgCfuUfcCfasasa | | |

Structure of Q339 is shown in FIG. 33.

As seen from the data summarized in Table 17, in vitro activity showed activity comparable in all cases. It is noteworthy that in vitro activity is recorded over very small period (one day) compared to in vivo RNAi activity. Interestingly, positive influence of morpolino monomer was visible only at day 7. Morpholino ring in addition to blocking the loading of sense strand also increase metabolic stability of the siRNA duplex. This can keep siRNA longer in the circulation and allow its slow release from endosomes. Thus, greater amounts of siRNA is available over longer period of time.

TABLE 17
IC₅₀ data on the on-target activity in
luciferase reporter assay for duplexes
that target ApoB

| Duplx | Alny | | | | |
|---|---|---|---|---|---|
| # | # | | | | IC50 |
| 1 | AD- | sense | UfsgsUfgAfc | 312 | 0.3767 |
| | 63716 | | AfaAfUfAfuG | | |
| | | | fgGfcAfuCfa | | |
| | | | AfL96 | | |
| | | antis | usUfsgAfuGf | 313 | |
| | | | cCfcAfuauUf | | |
| | | | uGfuCfaCfas | | |
| | | | asa | | |
| 2 | AD- | sense | UfsgsUfgAfc | 314 | 01634 |
| | 64559 | | AfaAfUfAfuG | | |
| | | | fgGfcAfuCfa | | |
| | | | AfL96 | | |
| | | antis | VPusUfsgAfu | 315 | |
| | | | GfcCfcAfuau | | |
| | | | UfuGfuCfaCf | | |
| | | | asasa | | |
| 3 | AD- | sense | Q339gsUfgAf | 316 | 0.2186 |
| | 870463 | | cAfaAfUfAfu | | |
| | | | GfgGfcAfuCf | | |
| | | | aAfL96 | | |
| | | antis | VPusUfsgAfu | 317 | |
| | | | GfcCfcAfuau | | |
| | | | UfuGfuCfaCf | | |
| | | | asasa | | |
| 4 | AD- | sense | Q339sgsUfgA | 318 | 0.3593 |
| | 870464 | | fcAfaAfUfAf | | |
| | | | uGfgGfcAfuC | | |
| | | | faAfL96 | | |
| | | antis | VPusUfsgAfu | 319 | |
| | | | GfcCfcAfuau | | |
| | | | UfuGfuCfaCf | | |
| | | | asasa | | |
| 5 | AD- | sense | Q339sgsUfgA | 320 | 0.6511 |
| | 870465 | | fcAfaAfUfAf | | |
| | | | uGfgGfcAfuC | | |
| | | | faAfL96 | | |
| | | antis | usUfsgAfuGf | 321 | |
| | | | cCfcAfuauUf | | |
| | | | uGfuCfaCfas | | |
| | | | asa | | |
| 6 | AD-8 | sense | Q339gUfgAfc | 322 | 0.251 |
| | 70466 | | AfaAfUfAfuG | | |
| | | | fgGfcAfuCfa | | |
| | | | AfL96 | | |
| | | antis | VPusUfsgAfu | 323 | |
| | | | GfcCfcAfuau | | |
| | | | UfuGfuCfaCf | | |
| | | | asasa | | |
| 7 | AD- | sense | Q339sgUfgAf | 324 | 02325 |
| | 870467 | | cAfaAfUfAfu | | |
| | | | GfgGfcAfuCf | | |
| | | | aAfL96 | | |
| | | antis | VPusUfsgAfu | 325 | |
| | | | GfcCfcAfuau | | |
| | | | UfuGfuCfaCf | | |
| | | | asasa | | |

TABLE 18
Additional exemplary duplexes that
target TTR

| | | SEQ | | | | |
|---|---|---|---|---|---|---|
| | Sequence | ID | Tar- | | Strand | Duplex |
| | (5′-3′) | NO. | get | | ID | ID |
| si-1 | a•a•cagu | 251 | TTR | S | A- | AD- |
| | GuUCUugc | | | | 128009 | 64958 |
| | ucuauaaL | | | | | |
| | u•U•auaG | 252 | | AS | A- | |
| | agcaagaA | | | | 126312 | |
| | cAcuguu• | | | | | |
| | u•u | | | | | |
| si-2 | a•a•cagu | 253 | TTR | S | A- | AD- |
| | GuUCUugc | | | | 128009 | 1479278 |
| | ucuauaaL | | | | | |
| | Mo2•U•au | 254 | | AS | A- | |
| | aGagcaag | | | | 2600326 | |
| | aAcAcugu | | | | | |
| | u•u•u | | | | | |
| si-3 | a•a•cagu | 255 | TTR | S | A- | |
| | GuUCUugc | | | | 128009 | |
| | ucuauaaL | | | | | |
| | Mo2•u•au | 256 | | AS | A- | |
| | aGagcaag | | | | 3831223 | |
| | aAcAcugu | | | | | |
| | u•u•u | | | | | |
| si-4 | a•a•cagu | 257 | TTR | S | A- | |
| | GuUCUugc | | | | 128009 | |
| | ucuauaaL | | | | | |
| | u•u•auaG | \|258 | | AS | A- | |
| | agcaagaA | | | | 137474 | |
| | cAcuguu• | | | | | |
| | u•u | | | | | |

Structure of Mo1, Mo2, and L are shown in FIG. 33.

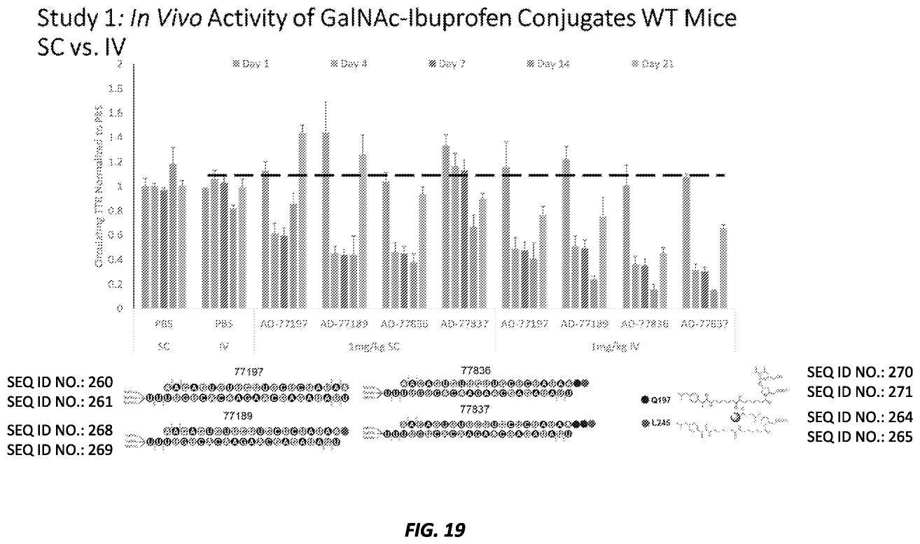

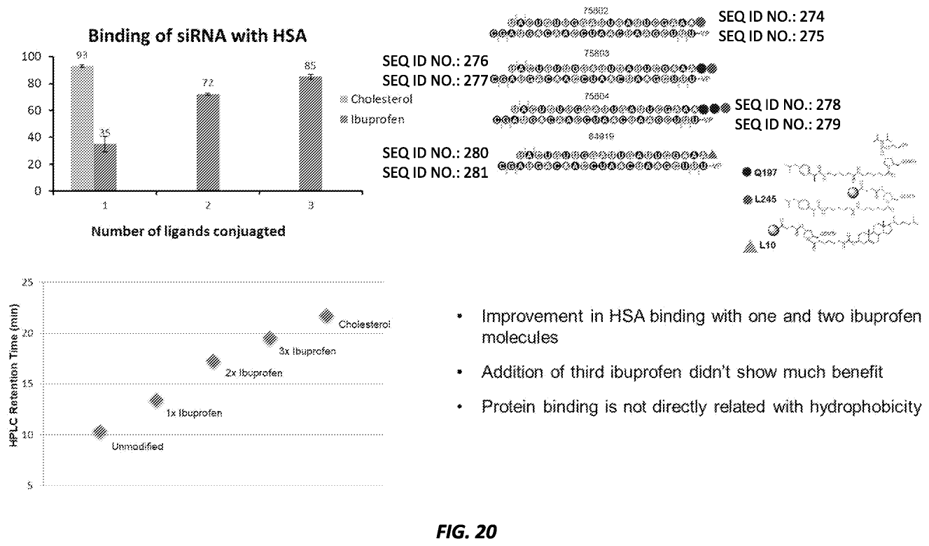

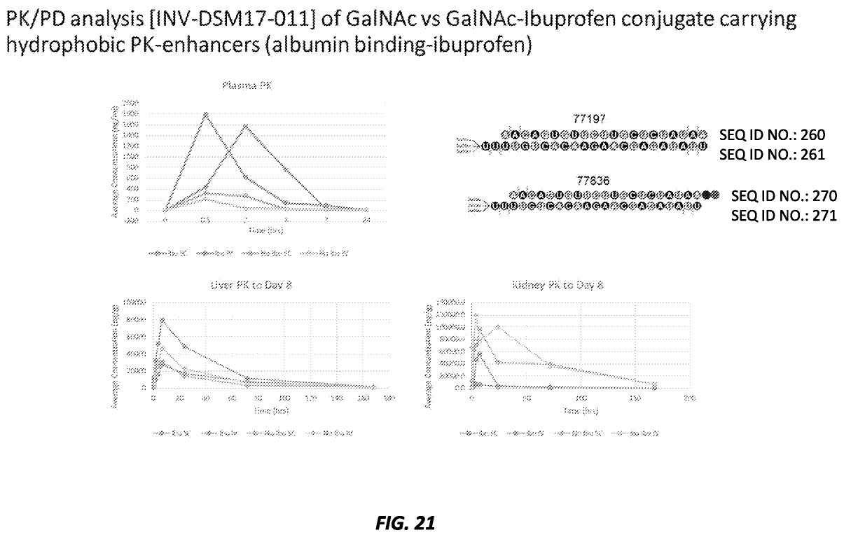

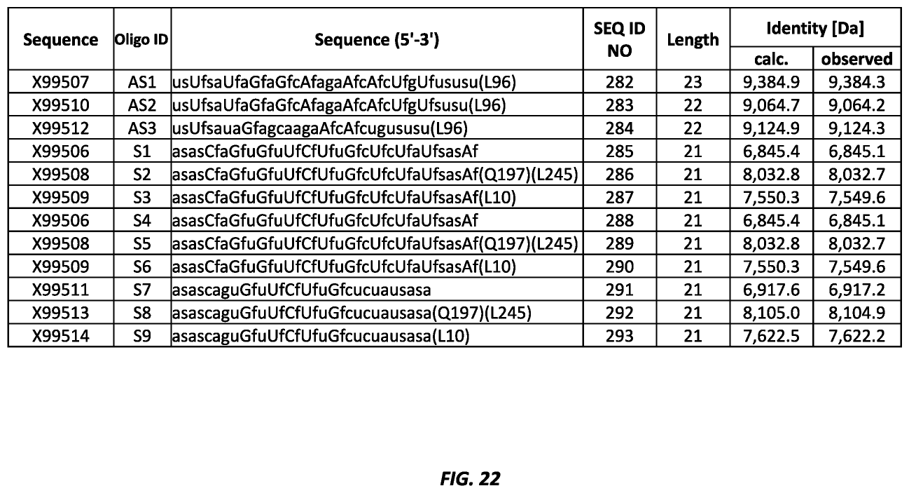

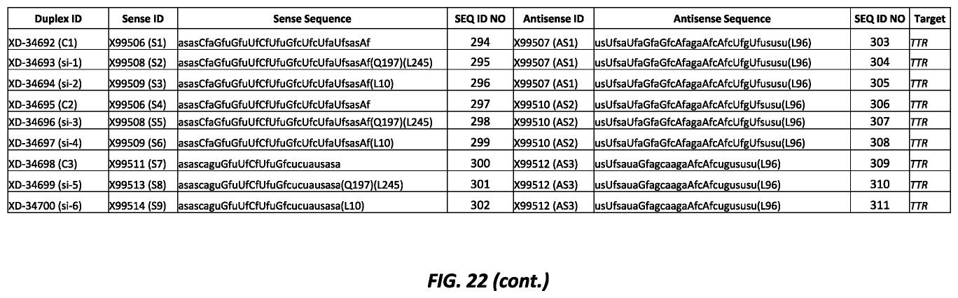

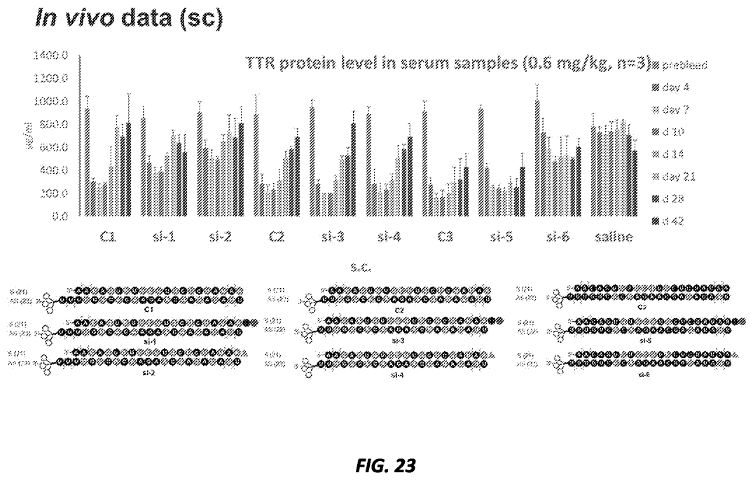

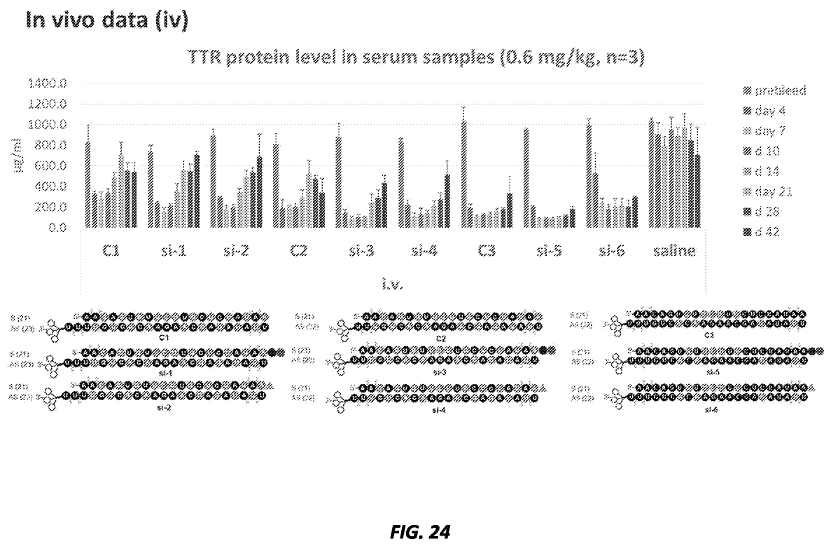

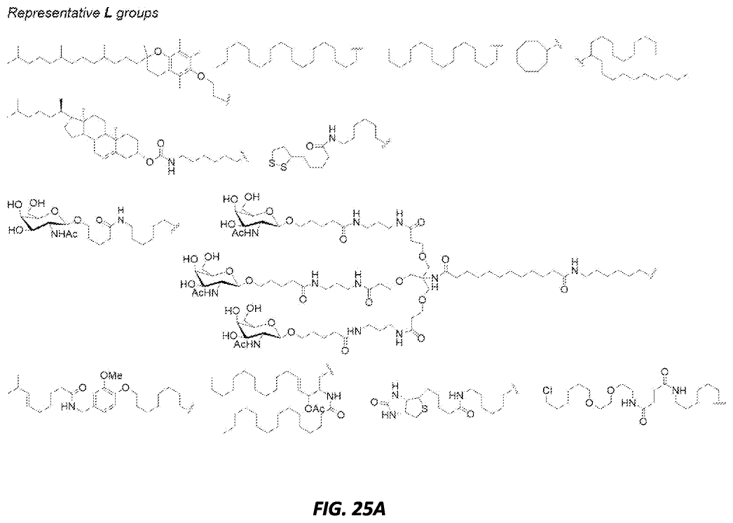

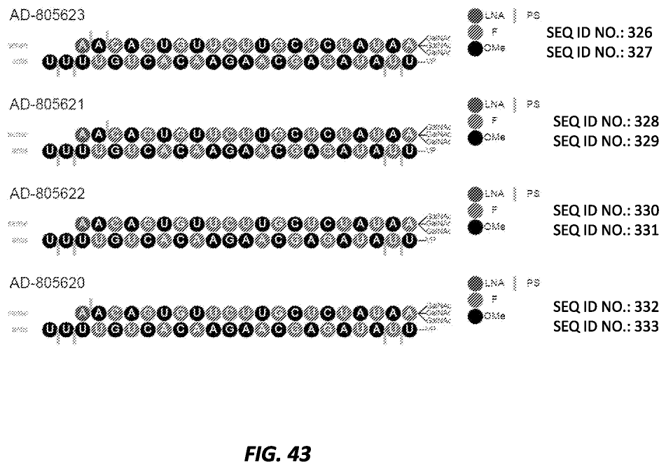

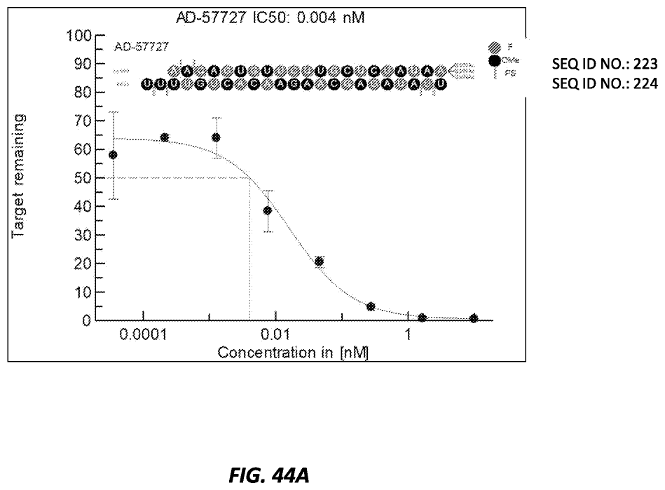

TABLE 19
SEQ ID NOs for sequences shown in FIGS. 19, 20 and

| | | | SEQ |
|---|---|---|---|
| | | | ID |
| | | | NO |
| For Figure 19 | | | |
| AD- | sense | AfsasCfaGfuGfuUfCfUfuGf | 260 |
| 77197 | | cUfcUfaUfsasAf | |
| | antis | usUfsaUfaGfaGfcAfagaAfc | 261 |
| | | AfcUfgUfususuL96 | |
| AD- | sense | AfsasCfaGfuGfuUfCfUfuGf | 262 |
| 77836 | | cUfcUfaUfaAf | |
| | | Q197L245 | |
| | antis | usUfsaUfaGfaGfcAfagaAfc | 263 |
| | | AfcUfgUfususuL96 | |
| AD- | sense | AfsasCfaGfuGfuUfCfUfuGf | 264 |
| 77837 | | cUfcUfaUfaAf | |
| | | Q197Q197L245 | |
| | antis | usUfsaUfaGfaGfcAfagaAfc | 265 |
| | | AfcUfgUfususuL96 | |
| AD- | sense | AfsasCfaGfuGfuUfCfUfuGf | 266 |
| 77197 | | cUfcUfaUfsasAf | |
| | antis | usUfsaUfaGfaGfcAfagaAfc | 267 |
| | | AfcUfgUfususuL96 | |
| AD- | sense | AfsasCfaGfuGfuUfCfUfuGf | 268 |
| 77189 | | cUfcUfaUfaAfL245 | |
| | antis | usUfsaUfaGfaGfcAfagaAfc | 269 |
| | | AfcUfgUfususuL96 | |
| AD- | sense | AfsasCfaGfuGfuUfCfUfuGf | 270 |
| 77836 | | cUfcUfaUfaAf | |
| | | Q197L245 | |
| | antis | usUfsaUfaGfaGfcAfagaAfc | 271 |
| | | AfcUfgUfususuL96 | |
| AD- | sense | AfsasCfaGfuGfuUfCfUfuGf | 272 |
| 77837 | | cUfcUfaUfaAf | |
| | | Q197Q197L245 | |
| | antis | usUfsaUfaGfaGfcAfagaAfc | 273 |
| | | AfcUfgUfususuL96 | |
| For Figure 20 | | | |
| AD-7 | sense | UfsasCfuGfuUfgGfAfUfuGf | 274 |
| 5802 | | aUfuCfgAfaAfL245 | |
| | antis | VPusUfsuCfgAfaUfcAfaucC | 275 |
| | | faAfcAfgUfasgsc | |
| AD- | sense | UfsasCfuGfuUfgGfAfUfuGf | 276 |
| 75803 | | aUfuCfgAfaAf | |
| | | Q197L245 | |
| | antis | VPusUfsuCfgAfaUfcAfaucC | 277 |
| | | faAfcAfgUfasgsc | |
| AD- | sense | UfsasCfuGfuUfgGfAfUfuGf | 278 |
| 75804 | | aUfuCfgAfaAf | |
| | | Q197Q197L245 | |
| | antis | VPusUfsuCfgAfaUfcAfaucC | 279 |
| | | faAfcAfgUfasgsc | |
| AD- | sense | UfsasCfuGfuUfgGfAfUfuGf | 280 |
| 64919 | | aUfuCfgAfaAfL10 | |
| | antis | VPusUfsuCfgAfaUfcAfaucC | 281 |
| | | faAfcAfgUfasgsc | |
| For Figure 43 | | | |
| AD- | sense | (Alns)asCfaGfuGfuUfCfUf | 326 |
| 805623 | | uGfcUfcUfaUfaAfL96 | |
| | antis | VPusUfsaUfaGfaGfcAfagaA | 327 |
| | | fcAfcUfgUfususu | |
| AD- | sense | (Aln)asCfaGfuGfuUfCfUfu | 328 |
| 805621 | | GfcUfcUfaUfaAfL96 | |
| | antis | VPusUfsaUfaGfaGfcAfagaA | 329 |
| | | fcAfcUfgUfususu | |
| AD- | sense | (Aln)aCfaGfuGfuUfCfUfuG | 330 |
| 805622 | | fcUfcUfaUfaAfL96 | |
| | antis | VPusUfsaUfaGfaGfcAfagaA | 331 |
| | | fcAfcUfgUfususu | |
| AD- | sense | (Alns)aCfaGfuGfuUfCfUfu | 332 |
| 805620 | | GfcUfcUfaUfaAfL96 | |
| | antis | VPusUfsaUfaGfaGfcAfcAfc | 333 |
| | | UfgUfususu | |

TABLE 20
Abbreviations used in sequences

| | |
|---|---|
| Af | 2′-deoxy-2′-fluoroadenosine-3′-phosphate |
| Afs | 2′-deoxy-2′-fluoroadenosine-3′-phosphorothioate |
| Cf | 2′-deoxy-2′-fluorocytidine-3′-phosphate |
| Cfs | 2′-deoxy-2′-fluorocytidine-3′-phosphorothioate |
| Gf | 2′-deoxy-2′-fluoroguanosine-3′-phosphate |
| Gfs | 2′-deoxy-2′-fluoroguanosine-3′-phosphorothioate |
| Uf | 2′-deoxy-2′-fluorouridine-3′-phosphate |
| Ufs | 2′-deoxy-2′-fluorouridine-3′-phosphorothioate |
| a | 2′-O-methyladenosine-3′-phosphate |
| as | 2′-O-methyladenosine-3′-phosphorothioate |
| c | 2′-O-methylcytidine-3′-phosphate |
| cs | 2′-O-methylcytidine-3′-phosphorothioate |
| g | 2′-O-methylguanosine-3′-phosphate |
| gs | 2′-O-methylguanosine-3′-phosphorothioate |
| t | 2′-O-methyl-5-methyluridine-3′-phosphate |
| ts | 2′-O-methyl-5-methyluridine-3′-phosphorothioate |
| u | 2′-O-methyluridine-3′-phosphate |
| us | 2′-O-methyluridine-3′-phosphorothioate |
| dA | 2′-deoxyadenosine-3′-phosphate (Deoxy-Adenosine) |
| dAs | 2′-deoxyadenosine-3′-phosphorothioate |
| dC | 2′-deoxycytidine-3′-phosphate (Deoxy-Cytidine) |
| dCs | 2′-deoxycytidine-3′-phosphorothioate |
| dG | 2′-deoxyguanosine-3′-phosphate (Deoxy-Guanosine) |
| dG | 2′-deoxyguanosine-3′-phosphorothioate |
| dT | 2′-deoxythymidine-3′-phosphate (Deoxy-Thymidine) |
| dTs | 2′-deoxythymidine-3′-phosphorothioate |
| VP | 5′-E-vinylphosphonate |
| s | Phosphorothioate linkage- indicates modification of 3′-phosphate for 3′- |
| | phosphorothioate on the preceding nucleotide |
| “•” symbol | Phosphorothioate linkages- indicates modification of 3′-phosphate for 3′- |
| | phosphorothioate on the preceding nucleotide |
| L96 | (2S,4R)-1-[29-[[2-(acetylamino)-2-deoxy-β-D-galactopyranosyl]oxy]-14,14- |
| | bis[[3-[[3-[[5-[[2-(acetylamino)-2-deoxy-β-D-galactopyranosyl]oxy]-1- |
| | oxopentyl]amino]propyl]amino]-3-oxopropoxy]methyl]-1,12,19,25-tetraoxo- |
| | 16-oxa-13,20,24-triazanonacos-1-yl]-4-hydroxy-2-hydroxymethylpyrrolidine |
| | 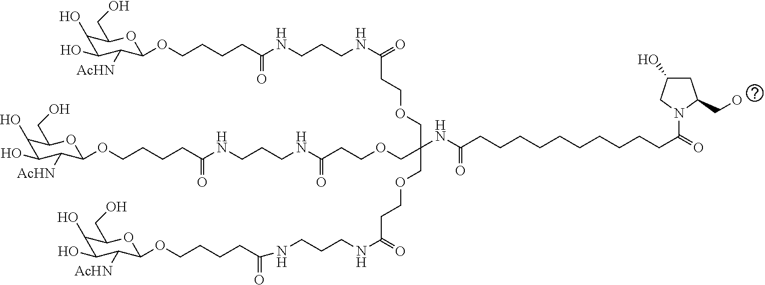 [SMILES: CC(=O)NC1C(OCCCCC(=O)NCCCNC(=O)CCOCC(COCCC(=O)NCCCNC(=O)CCCCOC2OC(CO)C(O)C(O)C2NC(C)=O)(COCCC(=O)NCCCNC(=O)CCCCOC2OC(CO)C(O)C(O)C2NC(C)=O)NC(=O)CCCCCCCCCCC(=O)N2C[C@H](O)C[C@H]2CO)OC(CO)C(O)C1O] |
| uL96 | 2′-O-methyluridine-3′-phosphate ((2S,4R)-1-[29-[[2-(acetylamino)-2-deoxy-β- |
| | D-galactopyranosyl]oxy]-14,14-bis[[3-[[3-[[5-[[2-(acetylamino)-2-deoxy-β-D- |
| | galactopyranosyl]oxy]-1-oxopentyl]amino]propyl]amino]-3- |
| | oxopropoxy]methyl]-1,12,19,25-tetraoxo-16-oxa-13,20,24-triazanonacos-1- |
| | yl]-4-hydroxy-3-pyrrolidinyl)methyl ester |
| | 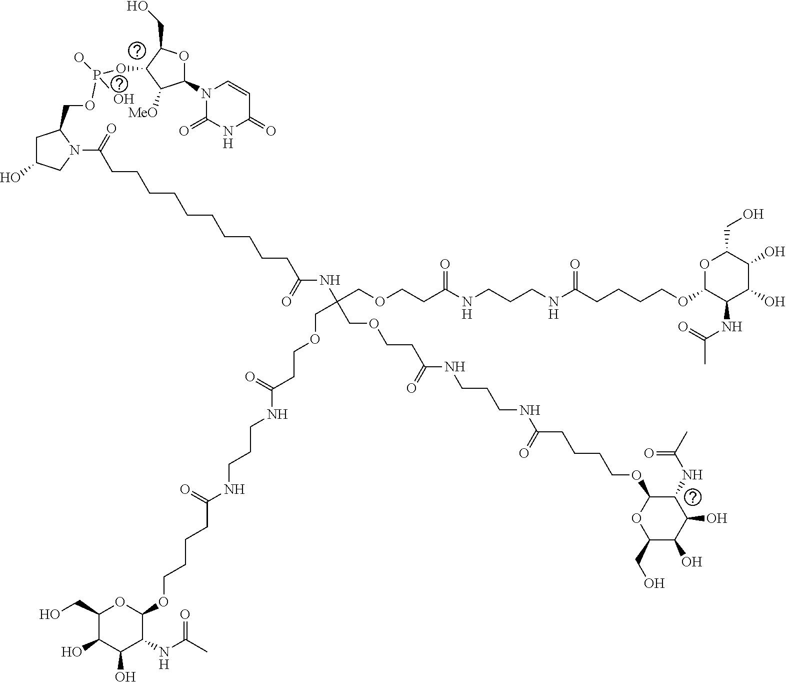 |
| aL96 | 2′-O-methyladenosine-3′-phosphate ((2S,4R)-1-[29-[[2-(acetylamino)-2- |
| | deoxy-β-D-galactopyranosyl]oxy]-14,14-bis[[3-[[3-[[5-[[2-(acetylamino)-2- |
| | deoxy-β-D-galactopyranosyl]oxy]-1-oxopentyl]amino]propyl]amino]-3- |
| | oxopropoxy]methyl]-1,12,19,25-tetraoxo-16-oxa-13,20,24-triazanonacos-1- |
| | yl]-4-hydroxy-2-pyrrolidinyl)methyl ester |
| cL96 | 2′-O-methylcytidine-3′-phosphate ((2S,4R)-1-[29-[[2-(acetylamino)-2-deoxy- |
| | β-D-galactopyranosyl]oxy]-14,14-bis[[3-[[3-[[5-[[2-(acetylamino)-2-deoxy-β- |
| | D-galactopyranosyl]oxy]-1-oxopentyl]amino]propyl]amino]-3- |
| | oxopropoxy]methyl]-1,12,19,25-tetraoxo-16-oxa-13,20,24-triazanonacos-1- |
| | yl]-4-hydroxy-2-pyrrolidinyl)methyl ester |
| gL96 | 2′-O-methylguanosine-3′-phosphate ((2S,4R)-1-[29-[[2-(acetylamino)-2- |
| | deoxy-β-D-galactopyranosyl]oxy]-14,14-bis[[3-[[3-[[5-[[2-(acetylamino)-2- |
| | deoxy-β-D-galactopyranosyl]oxy]-1-oxopentyl]amino]propyl]amino]-3- |
| | oxopropoxy]methyl]-1,12,19,25-tetraoxo-16-oxa-13,20,24-triazanonacos-1- |
| | yl]-4-hydroxy-2-pyrrolidinyl)methyl ester |
| AfL96 | 2′-deoxy-2′-fluoroadenosine-3′-phosphate ((2S,4R)-1-[29-[[2-(acetylamino)- |
| | 2-deoxy-β-D-galactopyranosyl]oxy]-14,14-bis[[3-[[3-[[5-[[2-(acetylamino)-2- |
| | deoxy-β-D-galactopyranosyl]oxy]-1-oxopentyl]amino]propyl]amino]-3- |
| | oxopropoxy]methyl]-1,12,19,25-tetraoxo-16-oxa-13,20,24-triazanonacos-1- |
| | yl]-4-hydroxy-2-pyrrolidinyl)methyl ester |
| CfL96 | 2′-deoxy-2′-fluorocytidine-3′-phosphate ((2S,4R)-1-[29-[[2-(acetylamino)-2- |
| | deoxy-β-D-galactopyranosyl]oxy]-14,14-bis[[3-[[3-[[5-[[2-(acetylamino)-2- |
| | deoxy-β-D-galactopyranosyl]oxy]-1-oxopentyl]amino]propyl]amino]-3- |
| | oxopropoxy]methyl]-1,12,19,25-tetraoxo-16-oxa-13,20,24-triazanonacos-1- |
| | yl]-4-hydroxy-2-pyrrolidinyl)methyl ester |
| GfL96 | 2′-deoxy-2′-fluoroguanosine-3′-phosphate ((2S,4R)-1-[29-[[2-(acetylamino)- |
| | 2-deoxy-β-D-galactopyranosyl]oxy]-14,14-bis[[3-[[3-[[5-[[2-(acetylamino)-2- |
| | deoxy-β-D-galactopyranosyl]oxy]-1-oxopentyl]amino]propyl]amino]-3- |
| | oxopropoxy]methyl]-1,12,19,25-tetraoxo-16-oxa-13,20,24-triazanonacos-1- |
| | yl]-4-hydroxy-2-pyrrolidinyl)methyl ester |
| UfL96 | 2′-deoxy-2′-fluorouridine-3′-phosphate ((2S,4R)-1-[29-[[2-(acetylamino)-2- |
| | deoxy-β-D-galactopyranosyl]oxy]-14,14-bis[[3-[[3-[[5-[[2-(acetylamino)-2- |
| | deoxy-β-D-galactopyranosyl]oxy]-1-oxopentyl]amino]propyl]amino]-3- |
| | oxopropoxy]methyl]-1,12,19,25-tetraoxo-16-oxa-13,20,24-triazanonacos-1- |
| | yl]-4-hydroxy-2-pyrrolidinyl)methyl ester |
| dTL96 | 2′-deoxythymidine-3′-phosphate ((2S,4R)-1-[29-[[2-(acetylamino)-2-deoxy- |
| | β-D-galactopyranosyl]oxy]-14,14-bis[[3-[[3-[[5-[[2-(acetylamino)-2-deoxy-β- |
| | D-galactopyranosyl]oxy]-1-oxopentyl]amino]propyl]amino]-3- |
| | oxopropoxy]methyl]-1,12,19,25-tetraoxo-16-oxa-13,20,24-triazanonacos-1- |
| | yl]-4-hydroxy-2-pyrrolidinyl)methyl ester |
| (Tgn) | Thymidine-glycol nucleic acid (GNA) S-Isomer |
| | 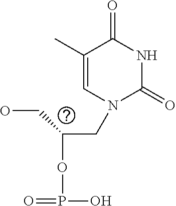 |
| Q339 | 5′-deoxy-5′-(N-morpholinyl)-2′-O,4′-C-methylene uridine-3′-phosphate |
| | 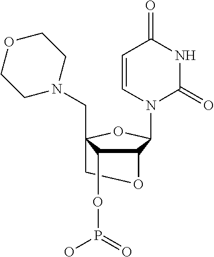 |
| Q339s | 5′-deoxy-5′-(N-morpholinyl)-2′-O,4′-C-methylene uridine-3′-phosphorothioate |
| | 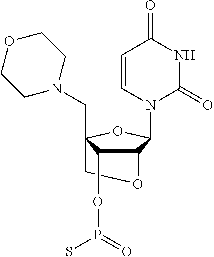 |
| Q340 | 5′-deoxy-5′-(N-morpholinyl)-2′-O,4′-C-methylene adenosine-3′-phosphate |
| | 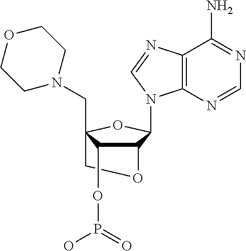 |
| Q340s | 5′-deoxy-5′-(N-morpholinyl)-2′-O,4′-C-methylene adenosine-3′- |
| | phosphorothioate |
| | 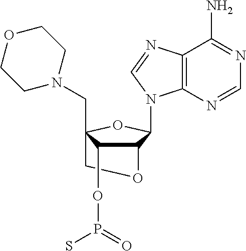 |
| (Aln) | 2′-O,4′-C-methylene adenosine-3′-phosphate |
| | 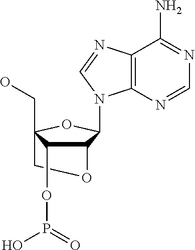 |
| (Alns) | 2′-O,4′-C-methylene adenosine-3′-phosphorothioate |
| | 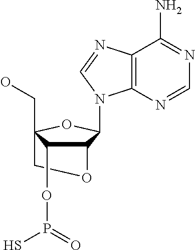 |
| Q197 | (2S,4R)-2-(hydroxymethyl)-1-(6-((S)-2-(4- |
| | isobutylphenyl)propanamido)hexanoyl)pyrrolidin-4-yl phosphate |
| | 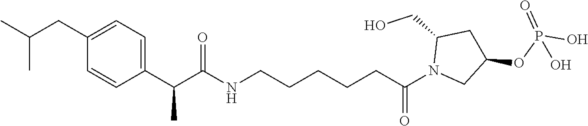 [SMILES: CC(C)Cc1ccc([C@H](C)C(=O)NCCCCCC(=O)N2C[C@H](OP(=O)(O)O)C[C@H]2CO)cc1] |
| L245 | (N-(6-((2S-(4-(2-methylpropyl)phenyl)-propan-1-oyl)amino)hexan-1-oyl)-4R- |
| | hydroxypyrrolidin-2S-yl)methanol |
| | 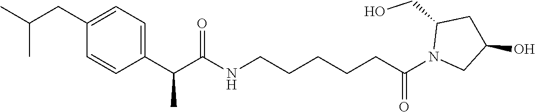 [SMILES: CC(C)Cc1ccc([C@H](C)C(=O)NCCCCCC(=O)N2C[C@H](O)C[C@H]2CO)cc1] |
| L10 | (N-(1-oxo-6-((3beta-cholest-5-en-3-yl)oxcarboxamido)hexyl)-4R- |
| | hydroxypyrrolidin-2S-yl)methanol |
| | 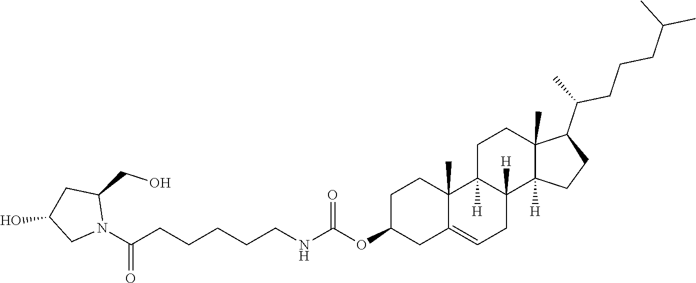 |
| Q197L245 | (4R-((((4R-(hydroxy)-1-(6-((S)-2-(4- |
| | isobutylphenyl)propanamido)hexanoyl)pyrrolidin-2S- |
| | yl)methoxy)hydroxyphosphoryl)oxy)-1-(6-((S)-2-(4- |
| | isobutylphenyl)propanamido)hexanoyl)pyrrolidin-2S-yl)methanol |
| | 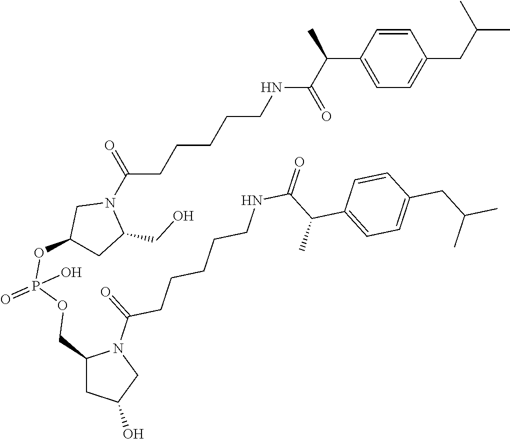 [SMILES: CC(C)Cc1ccc([C@H](C)C(=O)NCCCCCC(=O)N2C[C@H](O)C[C@H]2COP(=O)(O)O[C@@H]2C[C@@H](CO)N(C(=O)CCCCCNC(=O)[C@@H](C)c3ccc(CC(C)C)cc3)C2)cc1] |
| Q197Q197L245 | (4R-((((4R-((((4R-hydroxy-1-(6-((S)-2-(4- |
| | isobutylphenyl)propanamido)hexanoyl)pyrrolidin-2S- |
| | yl)methoxy)hydroxyphosphoryl)oxy)-1-(6-((S)-2-(4- |
| | isobutylphenyl)propanamido)hexanoyl)pyrrolidin-2S- |
| | yl)methoxy)hydroxyphosphoryl)oxy)-1-(6-((S)-2-(4- |
| | isobutylphenyl)propanamido)hexanoyl)pyrrolidin-2S-yl)methanol |
| | 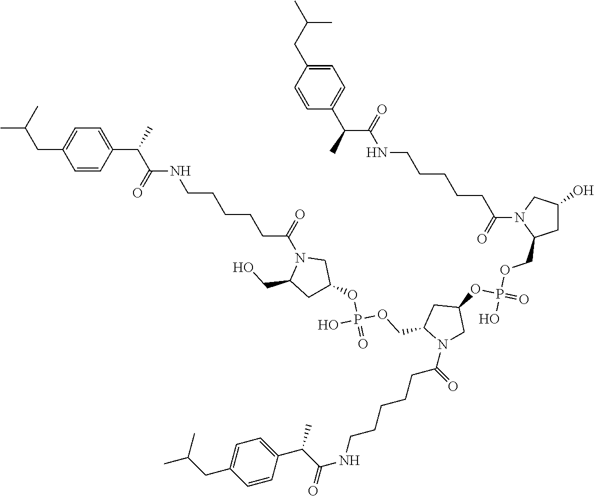 [SMILES: CC(C)Cc1ccc([C@H](C)C(=O)NCCCCCC(=O)N2C[C@H](O)C[C@H]2COP(=O)(O)O[C@@H]2C[C@@H](COP(=O)(O)O[C@@H]3C[C@@H](CO)N(C(=O)CCCCCNC(=O)[C@@H](C)c4ccc(CC(C)C)cc4)C3)N(C(=O)CCCCCNC(=O)[C@@H](C)c3ccc(CC(C)C)cc3)C2)cc1] |

*3′-terminal nucleotides are 3′-OH unless conjugated to (L) or otherwise indicated

All patents and other publications; including literature references, issued patents, published patent applications, and co-pending patent applications; cited throughout this application are expressly incorporated herein by reference for the purpose of describing and disclosing, for example, the methodologies described in such publications that might be used in connection with the technology described herein. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

What is claimed is:

1. A double-stranded RNA (dsRNA) comprising an antisense strand and a sense strand complementary to the antisense strand, wherein the antisense strand comprises at its 3′-end a first ligand, wherein the antisense strand comprises at least one nuclease resistant modification at its 3′-end and at least one nuclease resistant modification at its 5′-end, and wherein the dsRNA has a double-stranded region of at least about 15 base-pairs.

2. The dsRNA of claim 1, wherein the sense strand comprises at least one nuclease resistant modification at its 5′-end, optionally, the sense strand comprises at least one nuclease resistant modification at its 3′-end and at least one nuclease resistant modification at its 5′-end.

3. The dsRNA of claim 1, wherein the at least one nuclease resistant modification is a modified internucleoside linkage, a modified sugar moiety or a modified nucleobase, optionally, the at least one nuclease resistant modification is a phosphorothioate internucleoside linkage, a phosphorodithioate internucleoside linkage, a 2′-5′-linked nucleotide, or a L-nucleotide.

4. The dsRNA of claim 1, wherein the antisense strand comprises at least two, e.g., three, four, five, six or more phosphorothioate internucleoside linkages, optionally,
a. the antisense strand comprises a phosphorothioate internucleoside linkage between nucleotide positions 1 and 2, counting from the 3′-end of the antisense strand, and a phosphorothioate internucleoside linkage between nucleotide positions 1 and 2, counting from the 5′-end of the antisense strand;
b. the antisense strand comprises a phosphorothioate internucleoside linkage between positions 1 and 2, and between positions 2 and 3, counting from the 3′-end of the strand, and a phosphorothioate internucleoside linkage between positions 1 and 2, and between positions 2 and 3, counting from the 5′-end of the strand;
c. the antisense strand comprises a phosphorothioate internucleoside linkage between positions 1 and 2, between positions 2 and 3, and between positions 3 and 4, counting from the 3′-end of the strand, and a phosphorothioate internucleoside linkage between positions 1 and 2, counting from the 5′-end of the strand;
d. wherein the antisense strand comprises a phosphorothioate internucleoside linkage between positions 1 and 2, counting from the 3′-end of the strand, and a phosphorothioate internucleoside linkage between positions 1 and 2, and between positions 2 and 3, counting from the 5′-end of the strand; or
e. the antisense strand comprises a phosphorothioate internucleoside linkage between positions 1 and 2, counting from the 3′-end of the strand, and a phosphorothioate internucleoside linkage between positions 1 and 2, between positions 2 and 3, and between positions 3 and 4, counting from the 5′-end of the strand.

5. The dsRNA of claim 1, wherein the sense strand comprises at least one, e.g., two, three, four or more phosphorothioate internucleoside linkages, optionally,
a. the sense strand comprises a phosphorothioate internucleoside linkage between positions 1 and 2, counting from 5′-end of the strand;
b. the sense strand comprises a phosphorothioate internucleoside linkage between positions 1 and 2, counting from 5′-end of the strand, and between positions 1 and 2, counting from 3′-end of the strand;
c. the sense strand comprises a phosphorothioate internucleoside linkage between positions 1 and 2, and between positions 2 and 3, counting from 5′-end of the strand; or
d. the sense strand comprises a phosphorothioate internucleoside linkage between positions 1 and 2, and between positions 2 and 3, counting from 5′-end of the strand, and between positions 1 and 2, and between positions 2 and 3, counting from 3′-end of the strand.

6. The dsRNA of claim 1, wherein the first ligand is linked to 3′-hydroxyl of the nucleotide at position 1, counting from 3′-end of the antisense strand.

7. The dsRNA of claim 1, wherein the first ligand is linked to the 3′-end of the antisense strand via a linker, optionally, the linker is a hydrophobic linker or hydrophilic linker (e.g., polyethyleneglycol, such as —(CH₂CH₂O)_{w}—, where w is an integer, optionally w is an integer between 1 to 1000, e.g., an integer between 2 and 500).

8. The dsRNA of claim 6, where the linker is linked to the 3′-end of the antisense strand via a phosphodiester or phosphorothioate internucleoside linkage.

9. The dsRNA of claim 7 or 8, wherein the linker is from about 5 Angstroms to about 250 Angstroms in length and/or the linker has a chain length of at least 6 atoms.

10. The dsRNA of any one of claims 7-9, wherein the linker comprises a hydrophobic carrier connected to a carrier, optionally, the carrier comprises a hydrogen-bonding acceptor, optionally, the carrier comprises a pyrrolidine ring.

11. The dsRNA of claim 1, wherein the antisense strand is at least about 17 nucleotides in length, optionally, the antisense strand is about 19, about 20, about 21, about 22, about 23, about 24, about 25 or about 26 nucleotides in length, preferably the antisense strand is about 22, about 23 or about 25 nucleotides in length.

12. The dsRNA of claim 1, wherein the sense strand is at least about 15 nucleotides in length, optionally, the sense strand is about 19, about 20, about 21, about 22, about 23, about 24 or about 25 nucleotides in length, preferably the sense strand is about 21 nucleotides in length.

13. The dsRNA of claim 1, wherein:
(a) the sense strand is 15 nucleotides in length and the antisense strand is 18, 19, 20, 21, or 22 (e.g., 20) nucleotides in length;
(b) the sense strand is 19 nucleotides in length and the antisense strand is 19, 20, or 21 nucleotides in length;
(c) the sense strand is 20 nucleotides in length and the antisense strand is 20, 21, or 22 nucleotides in length;
(d) the sense strand is 21 nucleotides in length and the antisense strand is 21, 22, or 23 nucleotides in length; or
(e) the sense strand is 20-24 (e.g., 22) nucleotides in length and the antisense strand is 34-38 (e.g., 36) nucleotides in length.

14. The dsRNA of claim 1, wherein the dsRNA has a double-stranded region of at least about 15 base-pairs, optionally, the dsRNA has a double-stranded region of about 16, about 17, about 18, about 19, about 20, about 21, about 22, about 23, about 24, about 25 or more base-pairs, preferably, the dsRNA has a double-stranded region of about 21 base-pairs.

15. The dsRNA of claim 1, wherein the sense strand is about 21 nucleotides in length and the antisense strand is about 21, about 22, about 23, about 24 or about 25 nucleotides in length, and wherein the dsRNA comprises a double-stranded region of at least 18, e.g., 19, 20 or 21 base-pairs.

16. The dsRNA of any one of the preceding claims, wherein the dsRNA comprises at least one single-stranded overhang comprising 1-5 nucleotides (e.g., 1 or 2 nucleotides), optionally, the antisense strand comprises a single-stranded overhang at its 3′-end.

17. The dsRNA of claim 1, wherein the dsRNA comprises at least one blunt-end, optionally, the antisense strand comprises a blunt end at its 5′-end.

18. The dsRNA of claim 16 or 17, wherein the antisense strand comprises at least one nuclease resistant modification in the single-stranded overhang, and/or the antisense strand comprises at least one phosphorothioate internucleoside linkage in the single-stranded overhang.

19. The dsRNA of claim 1, wherein the ligand comprises GalNAc.

20. The dsRNA of any one of the preceding claims, wherein the ligand is 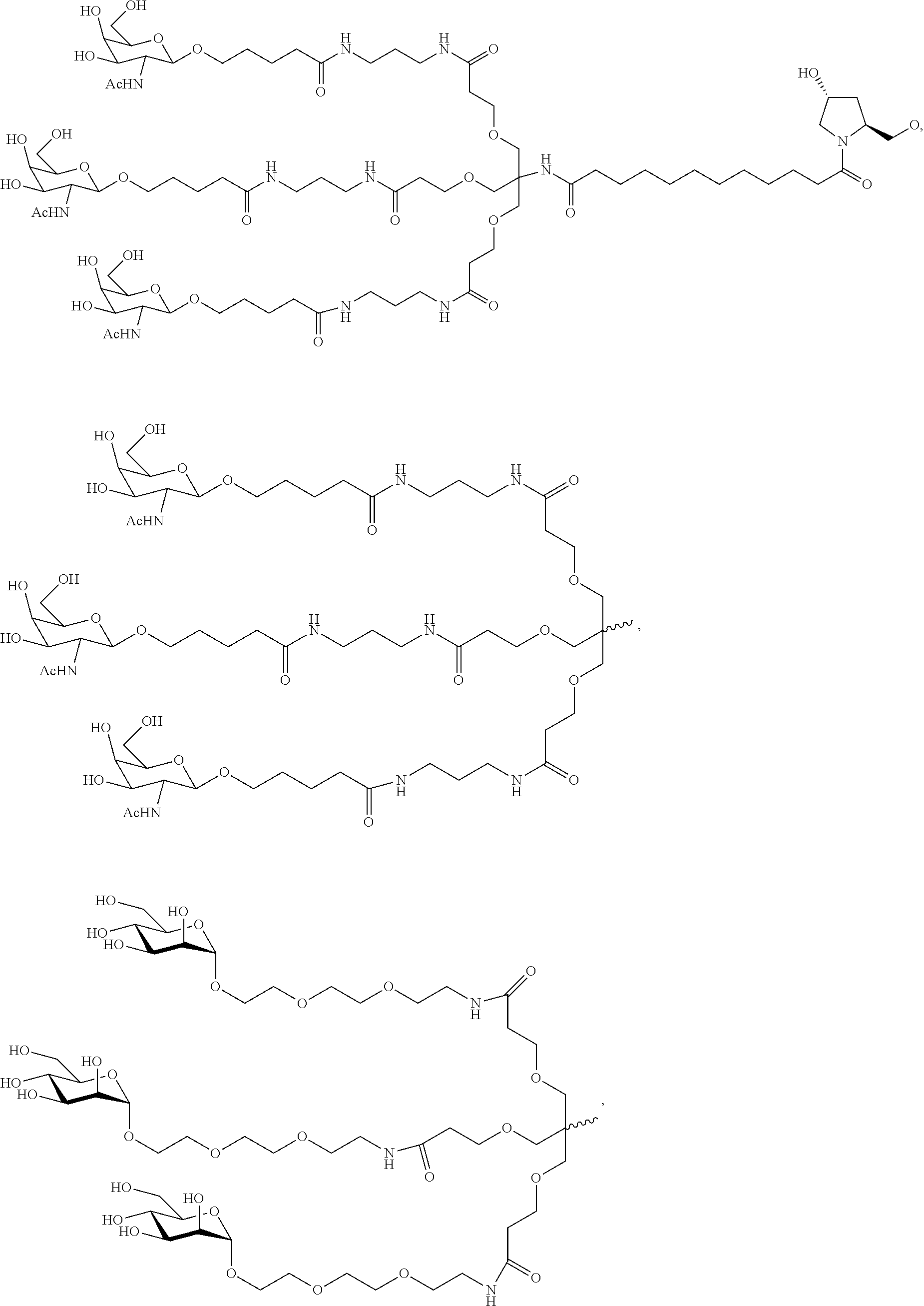 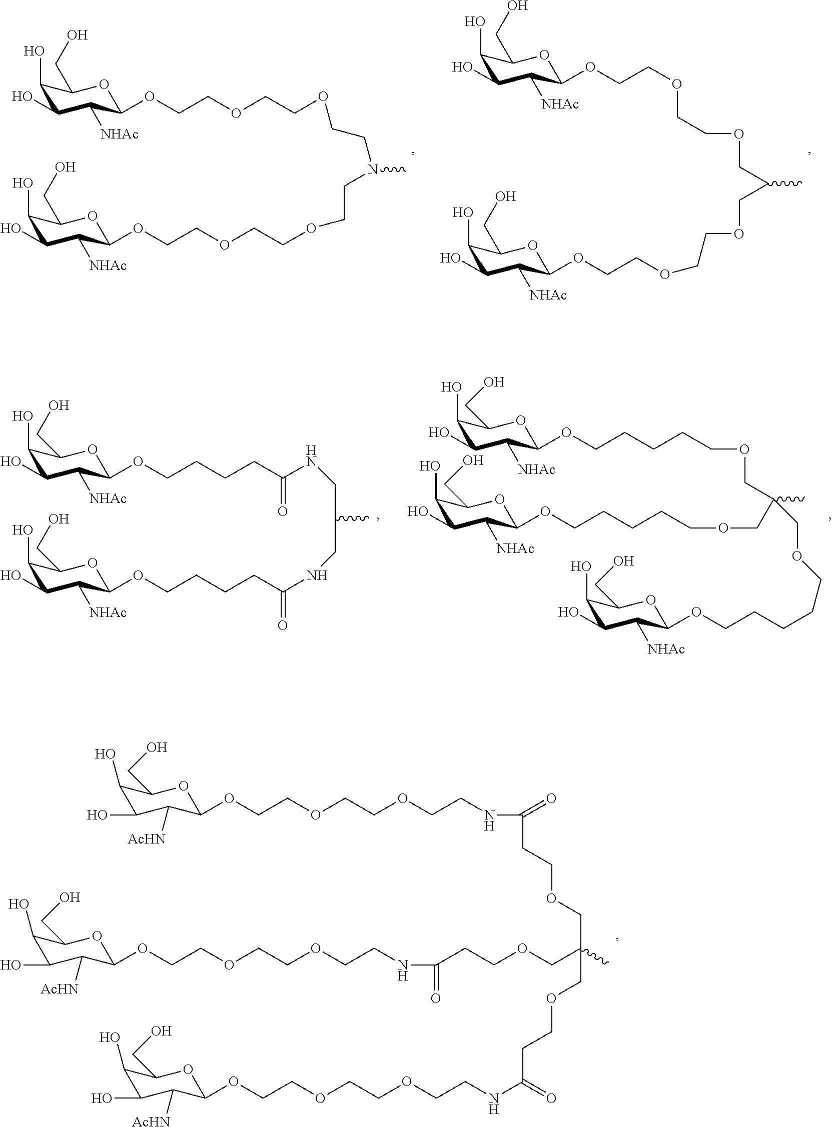 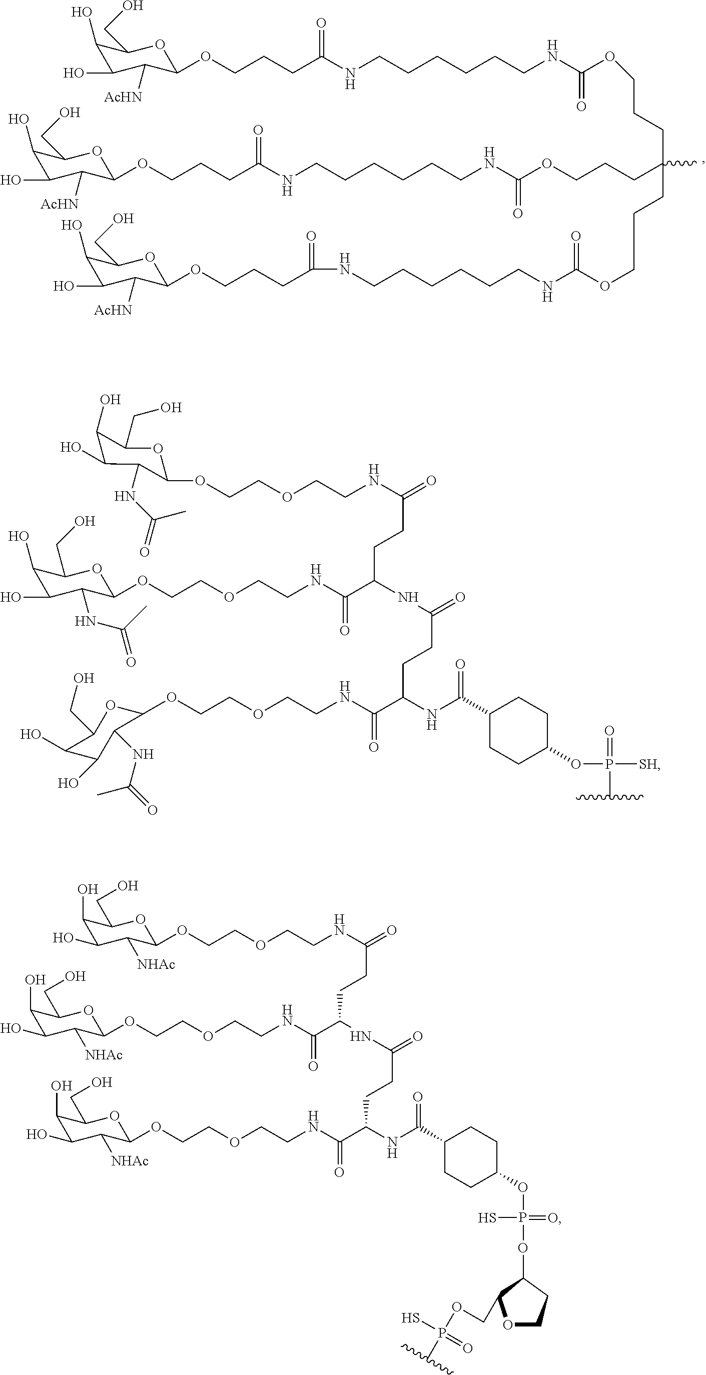 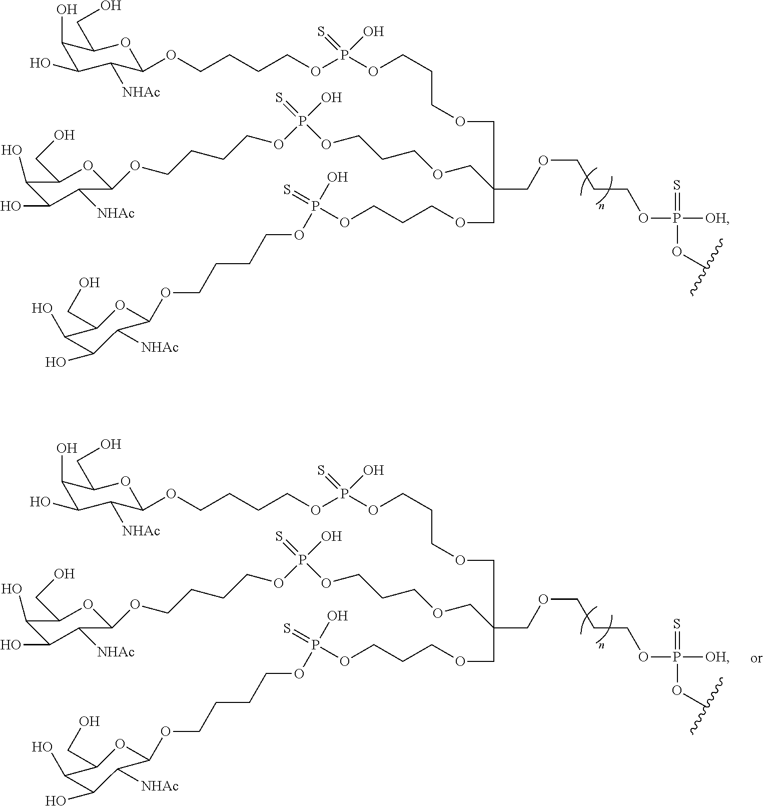 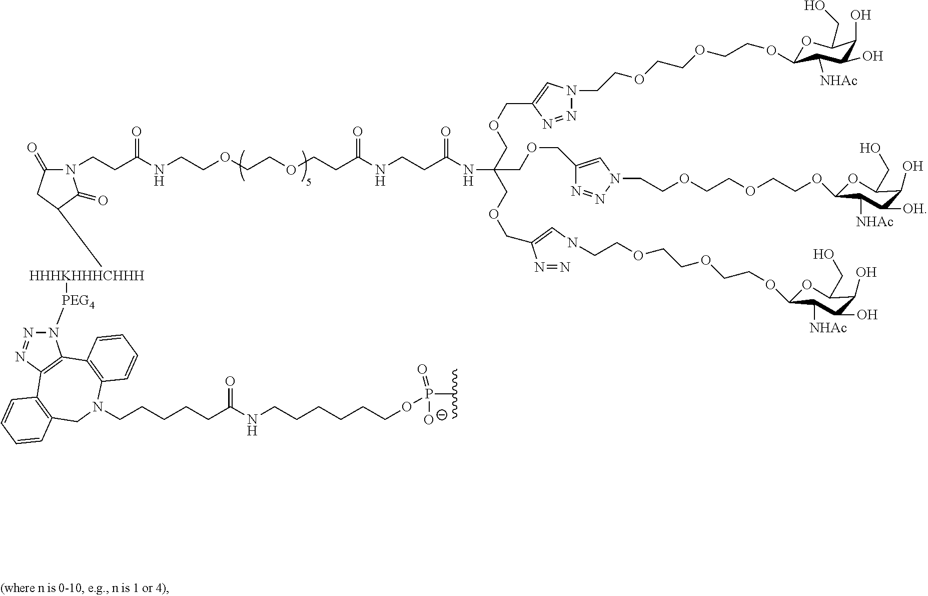
optionally the first ligand is

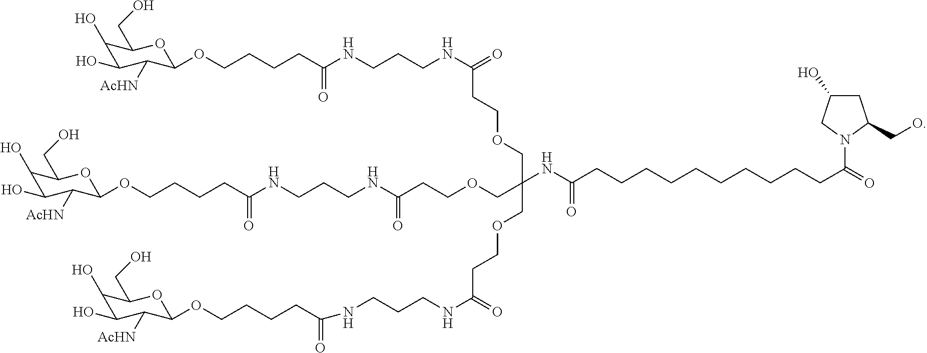

21. The dsRNA of any one of the preceding claims, wherein the dsRNA comprises a second ligand, optionally, the second ligand is linked to the sense strand.

22. The dsRNA of claim 21, wherein the second ligand is a PK modulator, a targeting ligand or an endosomolytic ligand, optionally, the second ligand is a PK modulator.

23. The dsRNA of any one of claims 21-22, wherein the second ligand binds a serum protein, e.g., serum albumin.

24. The dsRNA of any one of claims 21-23, wherein the second ligand comprises iodipamide, azapropazone, indomethacin, tiblone (TIB), 3-carboxy-4-methyl-5-propyl-2-furanpropanoic acid (CMPF), DIS, oxyphenbutazone, phenylbutazone, warfarin, indoxyl sulfate, diflunisal, halothane, ibuprofen, diazepam, propofol, or any combination thereof, optionally the second ligand comprises ibuprofen.

25. The dsRNA of any one of claims 21-24, wherein the first ligand comprises GalNac and the second ligand comprises ibuprofen, or the first ligand is GalNAc and the second ligand is a mannose receptor targeting ligand (e.g., multivalent mannose), or the first ligand is GalNAc and the second ligand is a folic acid ligand.

26. The dsRNA of any one of claims 21-25, wherein the dsRNA comprises at least one, e.g., 2, 3, 4, 5, 6, 7, 8, 9, 10 or more 2′-fluoro nucleotide.

27. The dsRNA of any one of claims 21-26, wherein the antisense strand comprises at least one, e.g., 2, 3, 4, 5 or more 2′-fluoro nucleotides, optionally
a. the antisense strand comprises a 2′-fluoro nucleotide at positions 2, 14 and 16, counting from the 5′-end of the antisense strand;
b. the antisense strand comprises a 2′-fluoro nucleotide at positions 2, 6, 14 and 16, counting from the 5′-end of the antisense strand;
c. the antisense strand comprises a 2′-fluoro nucleotide at positions 2, 6, 9, 14 and 16, counting from the 5′-end of the antisense strand; or
d. the antisense strand comprises a 2′-fluoro nucleotide at positions 2, 6, 8, 9, 14 and 16, counting from the 5′-end of the antisense strand.

28. The dsRNA of any one of claims 21-27, wherein the antisense strand comprises at least one, e.g., 2, 3, 4, 5 or more 2′-fluoro nucleotides, optionally
a. the sense strand comprises a 2′-fluoro nucleotide at positions 7, 9 and 11, counting from the 5′-end of the sense strand or at positions 11, 13 and 15, counting from the 3′-end of the sense strand;
b. the sense strand comprises a 2′-fluoro nucleotide at positions 7, 9, 10 and 11, counting from the 5′-end of the sense strand or at positions 11, 12, 13 and 15, counting from the 3′-end of the sense strand; or
c. the sense strand comprises a 2′-fluoro nucleotide at positions 9, 10, and 11, counting from the 5′-end of the sense strand or at positions 11, 12, and 13 counting from the 3′-end of the sense strand.

29. The dsRNA of any one of claims 21-28, wherein the antisense strand comprises at least one, e.g., 2, 3, 4, 5, 6, 7 or more DNA nucleotides, optionally
a. the antisense strand comprises a DNA nucleotide at positions 2, 5, 7, and 12, counting from the 5′-end of the antise8se strand, optionally the antisense strand further comprises a 2′-fluoro nucleotide at position 14, counting from of the 5-end of the antisense strand;
b. wherein the antisense strand comprises a DNA nucleotide at positions 2, 5, 7, 12, and 14 counting from the 5′-end of the antisense strand; or
c. the antisense strand comprises a DNA nucleotide at positions 2, 5, 7, 12, 14 and 16 counting from the 5′-end of the antisense strand.

30. The dsRNA of any one of claims 21-29, wherein the antisense strand comprises at least one 2′-OMe nucleotide, optionally, all remaining nucleotides in the antisense strand are 2′-OMe nucleotides.

31. The dsRNA of any one of claims 21-30, wherein the sense strand comprises at least one 2′-OMe nucleotide, optionally, all remaining nucleotides in the sense strand are 2′-OMe nucleotides.

32. The dsRNA of any one of claims 21-31, wherein the antisense strand comprises a phosphate group or a phosphate analog or derivative thereof at its 5′-end, optionally, the antisense strand comprises a vinylphosphonate (e.g., E-vinylphosphonate) group at its 5′-end.

33. The dsRNA of any one of claims 21-32, wherein:
a. the antisense strand comprises at least one, e.g., 2, 3, 4, 5 or more LNA or BNA nucleotides;
b. the sense strand comprises at least one, e.g., 2, 3, 4, 5 or more LNA or BNA nucleotides;
c. the antisense strand comprises at least one, e.g., 2, 3, 4, 5 or more CeNA nucleotides.
d. the antisense strand comprises at least one, e.g., 2, 3, 4, 5 or more thermally stabilizing modifications;
e. the sense strand comprises at least one, e.g., 2, 3, 4, 5 or more thermally stabilizing modifications;
f. the antisense strand comprises at least one, e.g., 2, 3, 4, 5 or more abasic nucleotides;
g. the sense strand comprises at least one, e.g., 2, 3, 4, 5 or more abasic nucleotides;
h. the antisense strand comprises at least one, e.g., 2, 3, 4, 5 or more 2′-deoxy nucleotides;
i. the sense strand comprises at least one, e.g., 2, 3, 4, 5 or more 2′-deoxy nucleotides;
j. the antisense strand comprises at least one, e.g., 2, 3, 4, 5 or more acyclic (e.g., unlocked nucleic acid (UNA) or glycol nucleic acid (GNA)) nucleotides;
k. the sense strand comprises at least one, e.g., 2, 3, 4, 5 or more acyclic (e.g., unlocked nucleic acid (UNA) or glycol nucleic acid (GNA)) nucleotides; and/or
l. the antisense strand comprises at least one thermally destabilizing modification, optionally, the antisense strand comprises at least one thermally destabilizing modification in the seed region (i.e., positions 2-9, e.g., position 6, 7, or 8, counting from the 5′-end) of the antisense strand, optionally, the thermally destabilizing modification is an abasic nucleotide, 2′-deoxy nucleotides, acyclic nucleotide (e.g., unlocked nucleic acid (UNA), glycol nucleic acid (GNA) or(S)-glycol nucleic acid (S-GNA)), a 2′-5′ linked nucleotide (3′-RNA), threose nucleotide (TNA), 2′ gem Me/F nucleotide, or mismatch with an opposing nucleotide in the other strand.

34. A compound of Formula (I): 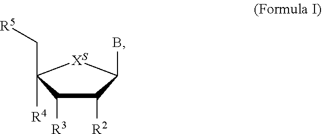
wherein:
B an optionally modified nucleobase;
X^{S} is O, CH₂, S, or NH;
R² is hydroxyl, protected hydroxyl, halogen, optionally substituted C₁₋₃₀ alkoxy (e.g., methoxy, 2-methoxyethoxy), alkoxyalkyl (e.g., 2-methoxyethyl), hydrogen, optionally substituted C₁₋₃₀ alkyl, optionally substituted C₂₋₃₀alkenyl, optionally substituted C₂₋₃₀alkynyl, alkoxyalkylamine, alkoxyoxycarboxylate, amino, alkylamino, dialkylamino, 5-8 membered heterocyclyl, —O—C₄₋₃₀alkyl-ON(CH₂R⁸)(CH₂R⁹), —O—C₄₋₃₀alkyl-ON(CH₂R⁸)(CH₂R⁹), phosphate group, reactive phosphorous group, a ligand, or a linker covalently bonded to one or more ligands;
R³ is a reactive phosphorous group, hydroxyl, protected hydroxyl, halogen, optionally substituted C₂₋₃₀alkynyl, optionally substituted C₁₋₃₀ alkoxy (e.g., methoxy, 2-methoxyethoxy), alkoxyalkyl (e.g., 2-methoxyethyl), hydrogen, optionally substituted C₁₋₃₀ alkyl, optionally substituted C₂₋₃₀alkenyl, alkoxyalkylamine, alkoxyoxycarboxylate, amino, alkylamino, dialkylamino, 5-8 membered heterocyclyl, —O—C₄₋₃₀alkyl-ON(CH₂R⁸)(CH₂R⁹), —O—C₄₋₃₀alkyl-ON(CH₂R⁸)(CH₂R⁹), phosphate group, a ligand, or a linker covalently bonded to one or more ligands;
R⁴ is hydrogen, optionally substituted C₁₋₆alkyl, optionally substituted C₂₋₆alkenyl, optionally substituted C₂₋₆alkynyl, or optionally substituted C₁₋₆alkoxy;
or R⁴ and R² taken together are 4′-C(R¹⁰R¹¹)ᵥ—Y-2′ or 4′-Y—C(R¹⁰R¹¹)ᵥ-2′;
Y is —O—, —CH₂—, —CH(Me)-, —C(CH₃)₂—, —S—, —N(R¹²)—, —C(O)—, —C(S)—, —S(O)—, —S(O)₂—, —OC(O)—, —C(O)O—, —N(R¹²)C(O)—, or —C(O)N(R¹²)—;
R¹⁰ and R¹¹ independently are H, optionally substituted C₁-C₆alkyl, optionally substituted C₂-C₆alkenyl or optionally substituted C₂-C₆alkynyl;
R¹² is hydrogen, optionally substituted C₁₋₃₀alkyl, optionally substituted C₁-C₃₀alkoxy, C₁₋₄haloalkyl, optionally substituted C₂₋₄alkenyl, optionally substituted C₂-4alkynyl, optionally substituted C₁₋₃₀alkyl-CO₂H, or a nitrogen-protecting group;
v is 1, 2 or 3; and
R⁵ is -L¹-R^{H}, —O—N(R¹³)R¹⁴,
where X^{P} is a phosphate group;
L¹ is a bond, -L³-, C₁₋₃₀alkylene, C₂₋₃₀alkenylene, C₂₋₃₀alkynylene, *-L³-C₁₋₃₀alkylene *-L³-C₂₋₃₀alkenylene, or *-L³-C₂₋₃₀alkynylene;
L³ is —O—, —N(R^{L3})—, —S—, —C(O)—, —S(O)—, —S(O)₂—, —P(X^{L3}) (Y^{L3}R^{L3B})—;
where R^{L3} is hydrogen, optionally substituted C₁₋₃₀alkyl,
optionally substituted C₁-C₃₀alkoxy, C₁₋₄haloalkyl,
optionally substituted C₂₋₄alkenyl, optionally substituted C₂-4alkynyl, optionally substituted C₁₋₃₀alkyl-CO₂H, or a nitrogen-protecting group;
X^{L2} is O or S;
Y^{L3} is O, S, NH, or a bond; and
R^{L3B} is H or optionally substituted alkyl; and
* is bond to R^{H};
and
R^{H} is 4-8 membered heterocyclyl comprising 1, 2 or 3 heteroatoms selected independently from N, O and S, and the heterocyclyl is optionally substituted with 1, 2, 3 or 4 independently selected substituents, and provided that the heterocyclyl comprises at least one nitrogen atom,
or R^{H} is 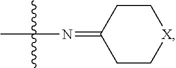
where X is O, NR^{L}, S, or CH₂; and R^{L} is hydrogen, a ligand, a linker covalently bonded to one or more ligands, aliphatic and aromatic alkyl, alkylester, alkylamine, dimethylamino alkyl, alkylether, alkylthioether, heteroaromatic alkyl, allyl, vinyl, alkyl groups functionalized with disulfide, oxime, ketone, acetal, hemiacetal, cleavable peptides, or cleavable sugars; and
R¹³ and R¹⁴ are independently -L²-R^{H2} or C₁-C₆alkyl, where:
L² is a linker; and
R^{H2} is 4-8 membered heterocyclyl comprising 1, 2 or 3 heteroatoms selected independently from N, O and S, and the heterocyclyl is optionally substituted with 1, 2, 3 or 4 independently selected substituents, and
provided that at least one of R¹³ and R¹⁴ is -L²-R^{H2}, and
provided that only one of R² and R³ is a reactive phosphorous group; and
R⁵ is not morpholin-4-yl unless R⁴ and R² taken together are 4′-C(R¹⁰R¹¹)ᵥ—Y-2′ or 4′-Y—C(R¹⁰R¹¹)ᵥ-2′.

35. The compound of claim 34, wherein R⁵ is -L¹-R^{H}, optionally, L¹ is L³ or C₁₋₃₀alkylene, optionally, L³ is O.

36. The compound of claim 35, wherein R^{H} is an optionally substituted 6-membered heterocyclyl comprising a nitrogen atom and 0, 1 or 2 additional heteroatoms selected independently from N, O and S, optionally
a. R^{H} is 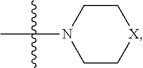
where X is O, NR^{L}, S, or CH₂; and R^{L} is hydrogen, a ligand, a linker covalently bonded to one or more ligands, aliphatic and aromatic alkyl, alkylester, alkylamine, dimethylamino alkyl, alkylether, alkylthioether, heteroaromatic alkyl, allyl, vinyl, alkyl groups functionalized with disulfide, oxime, ketone, acetal, hemiacetal, cleavable peptides, or cleavable sugars, optionally, X is O or NR^{L}, optionally R^{L} is hydrogen, a ligand or linker covalently bonded to one or more independently selected ligands;
b. R^{H} 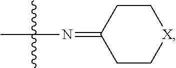
is optionally, X is O or The compound of claim 123, wherein X is NR^{L}, optionally, R^{L} is hydrogen, a ligand or linker covalently bonded to one or more independently selected ligands.

37. The compound of claim 34, wherein R⁵ is —O—N(R¹³)R¹⁴, optionally, one of R¹³ and R¹⁴ is -L²-R^{H2}, optionally, L² is a bond or an optionally substituted alkylene.

38. The compound of claim 34 or 37, wherein one of R¹³ and R¹⁴ is —(CH₂)ₘ—R^{H2} or 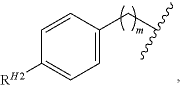
optionally R^{H2} is an optionally substituted 6-membered heterocyclyl comprising a nitrogen atom and 0, 1 or 2 additional heteroatoms selected independently from N, O and S, optionally
a. R^{H2} is 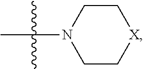
where X is O, NR^{L}, S, or CH₂; and R^{L} is hydrogen, a ligand, a linker covalently bonded to one or more ligands, aliphatic and aromatic alkyl, alkylester, alkylamine, dimethylamino alkyl, alkylether, alkylthioether, heteroaromatic alkyl, allyl, vinyl, alkyl groups functionalized with disulfide, oxime, ketone, acetal, hemiacetal, cleavable peptides, or cleavable sugars, optionally, X is O or NR^{L}, optionally, R^{L} is hydrogen, a ligand or linker covalently bonded to one or more independently selected ligands; or
b. R^{H2} is 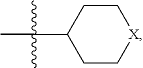
where X is O, NR^{L}, S, or CH₂; and R^{L} is hydrogen, a ligand, a linker covalently bonded to one or more ligands, aliphatic and aromatic alkyl, alkylester, alkylamine, dimethylamino alkyl, alkylether, alkylthioether, heteroaromatic alkyl, allyl, vinyl, alkyl groups functionalized with disulfide, oxime, ketone, acetal, hemiacetal, cleavable peptides, or cleavable sugars.

39. The compound of any one of claims 34-38, wherein one of R¹³ and R¹⁴ is an optionally substituted C₁-C₆alkyl, optionally, one of R¹³ and R¹⁴ is methyl.

40. The compound of any one of claims 34-39, wherein X^{S} is O or CH₂, optionally X^{S} is O.

41. The compound of any one of claims 34-40, wherein R³ is a reactive phosphorous group, hydroxyl, or protected hydroxyl, optionally, R³ is a reactive phosphorous group.

42. The compound of any one of claims 34-41, wherein R² is hydroxyl, protected hydroxyl, halogen, optionally substituted C₁₋₃₀ alkoxy (e.g., methoxy, 2-methoxyethoxy), alkoxyalkyl (e.g., 2-methoxyethyl), hydrogen, optionally substituted C₁₋₃₀ alkyl, optionally substituted C₂₋₃₀alkenyl, optionally substituted C₂₋₃₀alkynyl, alkoxyalkylamine, alkoxyoxycarboxylate, amino, alkylamino, dialkylamino, 5-8 membered heterocyclyl, —O—C₄₋₃₀alkyl-ON(CH₂R⁸) (CH₂R⁹), or —O—C₄₋₃₀alkyl-ON(CH₂R⁸) (CH₂R⁹); optionally, R² is hydroxyl, protected hydroxyl, halogen, optionally substituted C₁₋₃₀ alkoxy (e.g., methoxy, 2-methoxyethoxy), alkoxyalkyl (e.g., 2-methoxyethyl), hydrogen, amino, alkylamino, or dialkylamino; optionally, R² is hydrogen, hydroxyl, protected hydroxyl, fluoro, methoxy, ethoxy, or 2-methoxyethoxy; optionally, R² is hydrogen, hydroxyl, protected hydroxyl, fluoro, or methoxy.

43. The compound of any one of claims 34-42, wherein R² and R⁴ taken together are 4′-C(R¹⁰R¹¹)ᵥ—Y-2′ or 4′-Y—C(R¹⁰R¹¹)ᵥ-2′, optionally, R² and R⁴ taken together are 4′-C(R¹⁰R¹¹)ᵥ—Y-2′, wherein v is 1 or 2, optionally one of R¹⁰ and R¹¹ is H and the other is independently H or optionally substituted C₁-C₆alkyl, optionally, R² and R⁴ taken together are 4′-CH₂—O-2′.

44. The compound of any one of claims 34-40, wherein R² is a reactive phosphorous group, hydroxyl, or protected hydroxyl, optionally, R² is a reactive phosphorous group.

45. The compound of claim 44, wherein R³ is hydroxyl, protected hydroxyl, halogen, optionally substituted C₁₋₃₀ alkoxy (e.g., methoxy, 2-methoxyethoxy), alkoxyalkyl (e.g., 2-methoxyethyl), hydrogen, optionally substituted C₁₋₃₀ alkyl, optionally substituted C₂₋₃₀alkenyl, optionally substituted C₂₋₃₀alkynyl, alkoxyalkylamine, alkoxyoxycarboxylate, amino, alkylamino, dialkylamino, 5-8 membered heterocyclyl, —O—C₄₋₃₀alkyl-ON(CH₂R⁸)(CH₂R⁹), or —O—C₄₋₃₀alkyl-ON(CH₂R⁸)(CH₂R⁹); optionally, R³ is hydroxyl, protected hydroxyl, halogen, optionally substituted C₁₋₃₀ alkoxy (e.g., methoxy, 2-methoxyethoxy), alkoxyalkyl (e.g., 2-methoxyethyl), hydrogen, amino, alkylamino, or dialkylamino; optionally, R³ is hydrogen, hydroxyl, protected hydroxyl, fluoro, methoxy, ethoxy, or 2-methoxyethoxy; optionally R³ is hydrogen, hydroxyl, protected hydroxyl, fluoro, or methoxy.

46. The compound of any one of claim 34-42, 44 or 45, wherein R⁴ is H.

47. The compound of claim 34, wherein compound is selected from formulae (I-A)-(I-D): 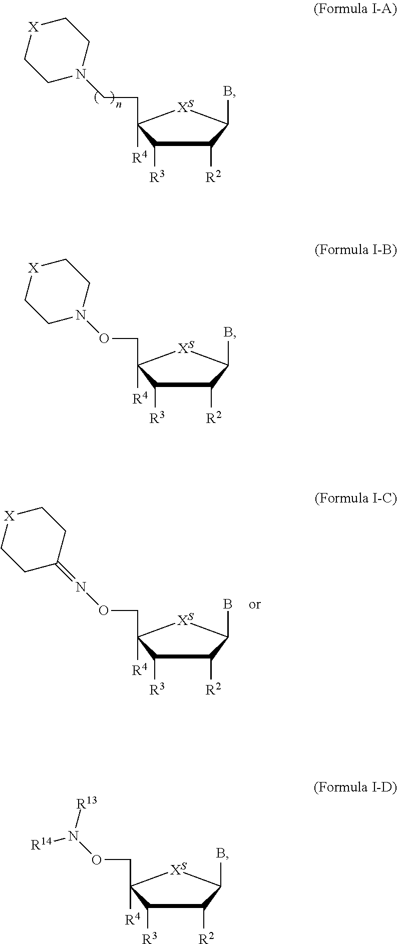
wherein:
n is 0 or an integer selected from 1 to 30 (e.g., n is 1, 2, 3, 4, 5, 6, 7, 8, 9, 10, 11, 12, 13, 14, 15, 16, 16, 17, 18, 19, 20, 21, 22, 23, 24, 25, 26, 27, 28, 29 or 30, such as n is 1, 2, 3, 4, 5 or 6, preferably n is 0 or 1);
R² is hydrogen, hydroxyl, protected hydroxyl, halogen, optionally substituted C₁₋₃₀ alkoxy (e.g., methoxy, 2-methoxyethoxy), alkoxyalkyl (e.g., 2-methoxyethyl), amino, alkylamino, or dialkylamino;
R³ is a reactive phosphorous group, hydroxyl, protected hydroxyl or a reactive phosphorous group;
R⁴ is hydrogen or R² and R⁴ taken together are 4′-C(R¹⁰R¹¹)ᵥ—Y-2′ or 4′-Y—C(R¹⁰R¹¹)ᵥ-2′.

48. The compound of claim 47, wherein X^{S} is O.

49. The compound of claim 47 or 48, wherein R³ is a reactive phosphorous group, hydroxyl or protected hydroxyl, optionally, R³ is a reactive phosphorous group (e.g., a phosphoramidite, such as 3′-[(2-cyanoethyl)-(N,N-diisopropyl)]-phosphoramidite, 3′-[(2-cyanoethyl)-(N,N-diisopropyl)]-phosphoramidite, or 3′-[(ß-thiobenzoylethyl)-(1-pyrrolidinyl)]-thiophosphoramidite).

50. The compound of any one of claims 47-49, wherein R² is hydrogen, hydroxyl, protected hydroxyl, halogen, optionally substituted C₁₋₃₀ alkoxy (e.g., methoxy, 2-methoxyethoxy), or alkoxyalkyl (e.g., 2-methoxyethyl), optionally, R² is hydrogen, hydroxyl, protected hydroxyl, fluoro, or methoxy.

51. The compound of any one of claims 47-50, wherein R⁴ is H.

52. The compound of any one of claims 47-50, wherein R² and R⁴ taken together are 4′-C(R¹⁰R¹¹)ᵥ—Y-2′ or 4′-Y—C(R¹⁰R¹¹)ᵥ-2′, optionally, R¹⁰ and R¹¹ is H and the other is independently H or optionally substituted C₁-C₆alkyl, optionally, R² and R⁴ taken together are 4′-CH₂—O-2′.

53. The compound of any one of claims 47-52, wherein one of R¹³ and R¹⁴ is 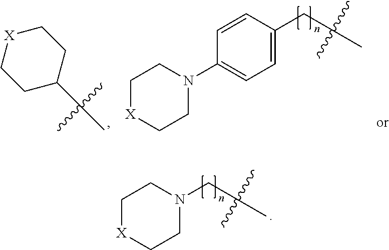
and the other of R¹³ and R¹⁴ is C₁-C₆alkyl,

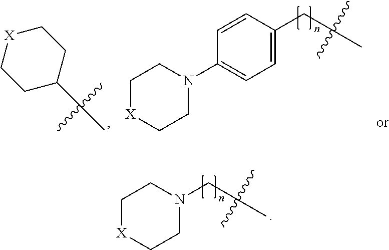

54. The compound of any one of claims 34-53, wherein one of R¹³ and R¹⁴ is C₁-C₆alkyl (e.g., methyl).

55. The compound of any one of claims 34-54 wherein X is O, S, CH₂, NH or NR^{L}, optionally, R^{L} is a ligand or linker covalently bonded to one or more independently selected ligands.

56. The compound of claim 34, wherein the compound is of Formula (I-E): 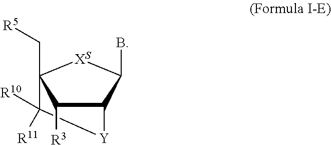
wherein:
R³ is a reactive phosphorous group, hydroxyl, or protected hydroxyl;
R⁵ is -L¹-R^{H}; and
X^{S}, B, Y, R¹⁰ and R¹¹ are as defined in claim 111.

57. The compound of claim 56, wherein compound is of Formula (I-Ea), (I-E¹) or (I-E²): 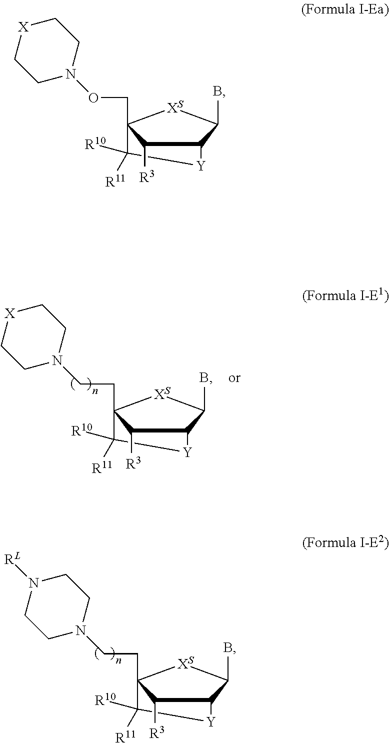
wherein
n is 0 or an integer selected from 1 to 30 (e.g., n is 1, 2, 3, 4, 5, 6, 7, 8, 9, 10, 11, 12, 13, 14, 15, 16, 16, 17, 18, 19, 20, 21, 22, 23, 24, 25, 26, 27, 28, 29 or 30, such as n is 1, 2, 3, 4, 5 or 6, preferably n is 0 or 1);
X is O, NR^{L}, S, or CH₂; and
R^{L} is H, a ligand, a linker covalently bonded to one or more ligands, aliphatic and aromatic alkyl, alkylester, alkylamine, dimethylamino alkyl, alkylether, alkylthioether, heteroaromatic alkyl, allyl, vinyl, alkyl groups functionalized with disulfide, oxime, ketone, acetal, hemiacetal, cleavable peptides, or cleavable sugars, and optionally, the compound is of Formula (I-Eb) or (I-Ec):

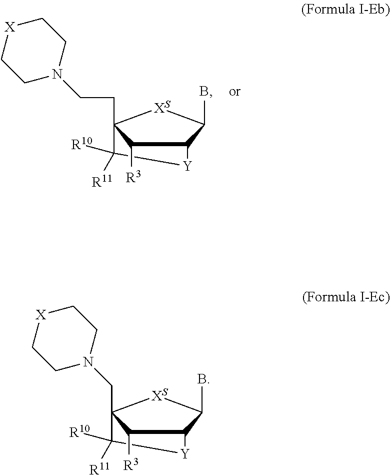

58. The compound of claim 56 or 57, wherein X is O or CH₂, optionally, X is O.

59. The compound of any one of claims 56-58, wherein Y is O.

60. The compound of any one of claims 56-59, wherein one of R¹⁰ is H and the other is H or C₁₋₆alkyl (e.g., methyl).

61. The compound of any one of claims 56-60, wherein X^{S} is O.

62. The compound of any one of claims 56-61, wherein R³ is a reactive phosphorous group, hydroxyl or protected hydroxyl, optionally, R³ is a reactive phosphorous group (e.g., a phosphoramidite, such as 3′-[(2-cyanoethyl)-(N,N-diisopropyl)]-phosphoramidite, 3′-[(2-cyanoethyl)-(N,N-diisopropyl)]-phosphoramidite, or 3′-[(ß-thiobenzoylethyl)-(1-pyrrolidinyl)]-thiophosphoramidite).

63. The compound of claim 56, wherein the compound is of Formula (I-Ed), (I-Ee), (I-E³) or (I-E⁴): 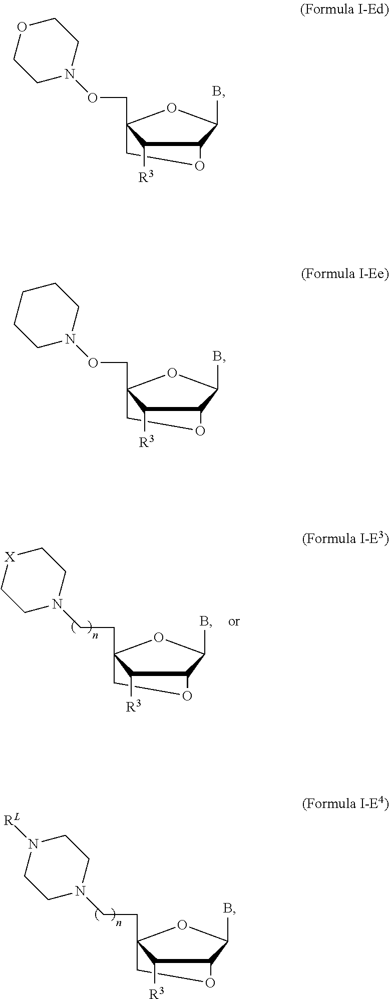
where n is 0 or an integer selected from 1 to 30 (e.g., n is 1, 2, 3, 4, 5, 6, 7, 8, 9, 10, 11, 12, 13, 14, 15, 16, 16, 17, 18, 19, 20, 21, 22, 23, 24, 25, 26, 27, 28, 29 or 30, such as n is 1, 2, 3, 4, 5 or 6, preferably n is 0 or 1); and
R^{L} is H, a ligand, a linker covalently bonded to one or more ligands, aliphatic and aromatic alkyl, alkylester, alkylamine, dimethylamino alkyl, alkylether, alkylthioether, heteroaromatic alkyl, allyl, vinyl, alkyl groups functionalized with disulfide, oxime, ketone, acetal, hemiacetal, cleavable peptides, or cleavable sugars, and
optionally, the compound is of Formula (I-Ef) or (I-Eg):

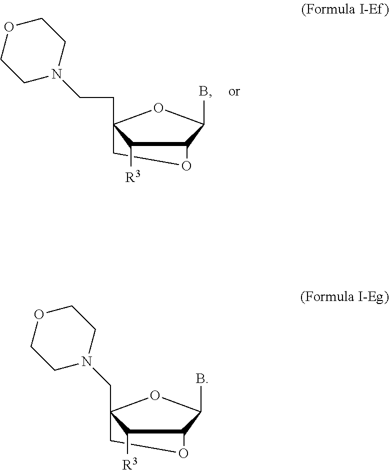

64. An oligonucleotide prepared using a compound of any one of claims 34-63.

65. An oligonucleotide comprising at least one nucleoside of Formula (II): 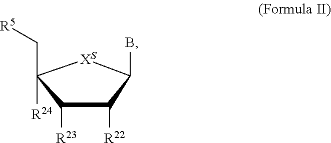
wherein:
B an optionally modified nucleobase;
X^{S} is O, CH₂, S, or NH;
R²² is hydroxyl, protected hydroxyl, halogen, optionally substituted C₁₋₃₀ alkoxy (e.g., methoxy, 2-methoxyethoxy), alkoxyalkyl (e.g., 2-methoxyethyl), hydrogen, optionally substituted C₁₋₃₀ alkyl, optionally substituted C₂₋₃₀alkenyl, optionally substituted C₂₋₃₀alkynyl, alkoxyalkylamine, alkoxyoxycarboxylate, amino, alkylamino, dialkylamino, 5-8 membered heterocyclyl, —O—C₄₋₃₀alkyl-ON(CH₂R⁸)(CH₂R⁹), —O—C₄₋₃₀alkyl-ON(CH₂R⁸)(CH₂R⁹), a ligand, a linker covalently bonded to one or more ligands or a bond to an internucleotide linkage to a subsequent nucleoside;
R²³ is a bond to an internucleotide linkage to a subsequent nucleoside, hydroxyl, protected hydroxyl, halogen, optionally substituted C₂₋₃₀alkynyl, optionally substituted C₁₋₃₀ alkoxy (e.g., methoxy, 2-methoxyethoxy), alkoxyalkyl (e.g., 2-methoxyethyl), hydrogen, optionally substituted C₁₋₃₀ alkyl, optionally substituted C₂₋₃₀alkenyl, alkoxyalkylamine, alkoxyoxycarboxylate, amino, alkylamino,
dialkylamino, 5-8 membered heterocyclyl, —O—C₄₋₃₀alkyl-ON(CH₂R⁸)(CH₂R⁹), —O—C₄₋₃₀alkyl-ON(CH₂R⁸)(CH₂R⁹), phosphate group, a ligand, or a linker covalently bonded to one or more ligands;
R²⁴ is hydrogen, optionally substituted C₁₋₆alkyl, optionally substituted C₂₋₆alkenyl, optionally substituted C₂₋₆alkynyl, or optionally substituted C₁₋₆alkoxy;
or R²² and R²⁴ taken together are 4′-C(R¹⁰R¹¹)ᵥ—Y-2′ or 4′-Y—C(R¹⁰R¹¹)ᵥ-2′;
Y is —O—, —CH₂—, —CH(Me)-, —C(CH₃)₂—, —S—, —N(R¹²)—, —C(O)—, —C(S)—, —S(O)—, —S(O)₂—, —OC(O)—, —C(O)O—, —N(R¹²)C(O)—, or —C(O)N(R¹²)—;
R¹⁰ and R¹¹ independently are H, optionally substituted C₁-C₆alkyl, optionally substituted C₂-C₆alkenyl or optionally substituted C₂-C₆alkynyl;
R¹² is hydrogen, optionally substituted C₁₋₃₀alkyl, optionally substituted C₁-C₃₀alkoxy, C₁₋₄haloalkyl, optionally substituted C₂₋₄alkenyl, optionally substituted C₂₋₄alkynyl, optionally substituted C₁₋₃₀alkyl-CO₂H, or a nitrogen-protecting group;
v is 1, 2 or 3; and
R⁵ is -L¹-R^{H}, —O—N(R¹³)R¹⁴,
where X^{P} is a phosphate group;
L¹ is a bond, -L³-, C₁₋₃₀alkylene, C₂₋₃₀alkenylene, C₂₋₃₀alkynylene, *-L³-C₁₋₃₀alkylene *-L³-C₂₋₃₀alkenylene, or *-L³-C₂₋₃₀alkynylene;
L³ is —O—, —N(R^{L3})—, —S—, —C(O)—, —S(O)—, —S(O)₂—, —P(X^{L3}) (Y^{L3}R^{L3B})—;
where R¹³ is hydrogen, optionally substituted C₁₋₃₀alkyl,
optionally substituted C₁-C₃₀alkoxy, C₁₋₄haloalkyl,
optionally substituted C₂₋₄alkenyl, optionally substituted C₂₋₄alkynyl, optionally substituted C₁₋₃₀alkyl-CO₂H, or a nitrogen-protecting group;
X^{L2} is O or S;
Y^{L3} is O, S, NH, or a bond; and
R^{L3B} is H or optionally substituted alkyl; and
* is bond to R^{H};
and
R^{H} is 4-8 membered heterocyclyl comprising 1, 2 or 3 heteroatoms selected independently from N, O and S, and the heterocyclyl is optionally substituted with 1, 2, 3 or 4 independently selected substituents, and provided that the heterocyclyl comprises at least one nitrogen atom, or R^{H} is, 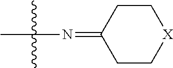
where X is O, NR^{L}, S, or CH₂; and R^{L} is hydrogen, a ligand, a linker covalently bonded to one or more ligands, aliphatic and aromatic alkyl, alkylester, alkylamine, dimethylamino alkyl, alkylether, alkylthioether, heteroaromatic alkyl, allyl, vinyl, alkyl groups functionalized with disulfide, oxime, ketone, acetal, hemiacetal, cleavable peptides, or cleavable sugars; and
R¹³ and R¹⁴ are independently -L²-R^{H2}, where:
L² is a linker; and
R^{H2} is 4-8 membered heterocyclyl comprising 1, 2 or 3 heteroatoms selected independently from N, O and S, and the heterocyclyl is optionally substituted with 1, 2, 3 or 4 independently selected substituents, and
provided that at least one of R¹³ and R¹⁴ is -L²-R^{H2}, and
provided that one of R²² and R²³ is a bond to an internucleotide linkage to a subsequent nucleoside and only one of R²² and R²³ is a bond to an internucleotide linkage to a subsequent nucleoside.

66. The oligonucleotide of claim 65, wherein the nucleoside of Formula (II) is selected from formulae (II-A)-(II-D): 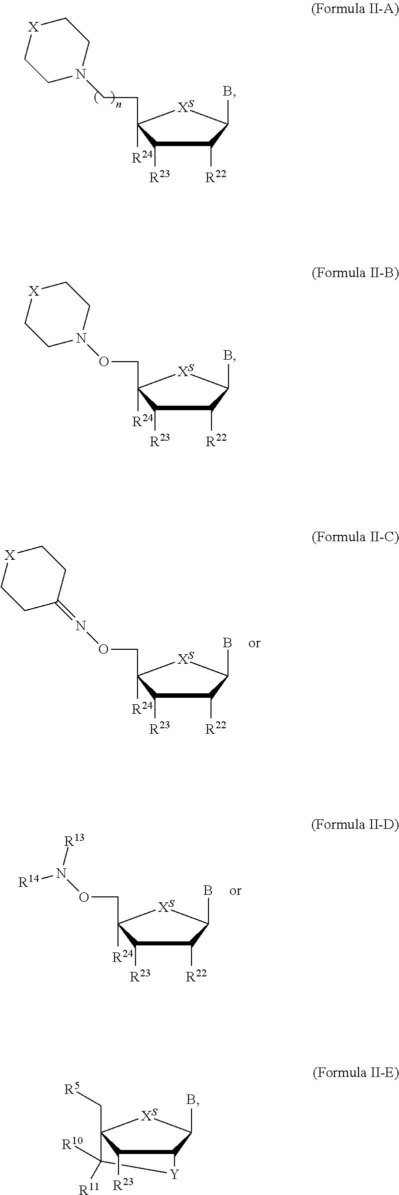
wherein:
R⁵ is -L¹-R^{H};
R²² is hydrogen, hydroxyl, protected hydroxyl, halogen, optionally substituted C₁₋₃₀ alkoxy (e.g., methoxy, 2-methoxyethoxy), alkoxyalkyl (e.g., 2-methoxyethyl), amino, alkylamino, or dialkylamino;
R²³ is a bond to an internucleotide linkage to a subsequent nucleoside;
R²⁴ is hydrogen or R²² and R²⁴ taken together are 4′-C(R¹⁰R¹¹)ᵥ—Y-2′ or 4′-Y—C(R¹⁰R¹¹)ᵥ-2′; and
X^{S}, B, Y, R¹⁰ and R¹¹ are as defined in claim 34.

67. The oligonucleotide of claim 66, wherein the nucleoside is of Formula (II-Ea), (II-E′) or (II-E″: 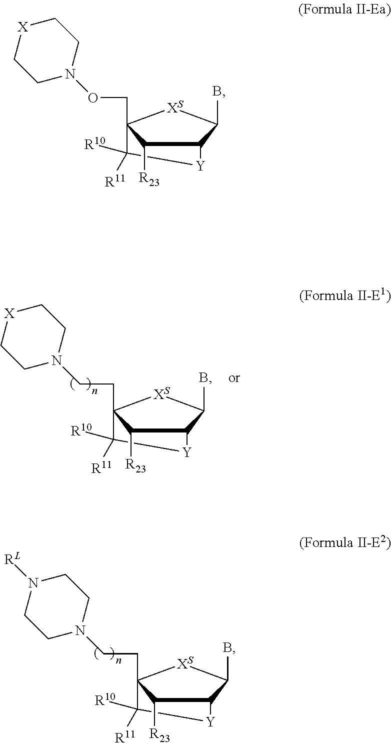
wherein
n is 0 or an integer selected from 1 to 30 (e.g., n is 1, 2, 3, 4, 5, 6, 7, 8, 9, 10, 11, 12, 13, 14, 15, 16, 16, 17, 18, 19, 20, 21, 22, 23, 24, 25, 26, 27, 28, 29 or 30, such as n is 1, 2, 3, 4, 5 or 6, preferably n is 0 or 1);
X is O, NR^{L}, S, or CH₂; and
R^{L} is H, a ligand, a linker covalently bonded to one or more ligands, aliphatic and aromatic alkyl, alkylester, alkylamine, dimethylamino alkyl, alkylether, alkylthioether, heteroaromatic alkyl, allyl, vinyl, alkyl groups functionalized with disulfide, oxime, ketone, acetal, hemiacetal, cleavable peptides, or cleavable sugars, and optionally, the compound is of Formula (II-Eb) or (II-Ec):

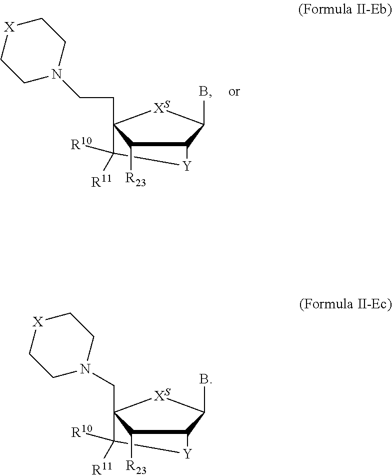

68. The oligonucleotide of claim 67, wherein the nucleoside is of Formula (II-Ed), (II-Ee), (II-E3) or (II-E⁴): 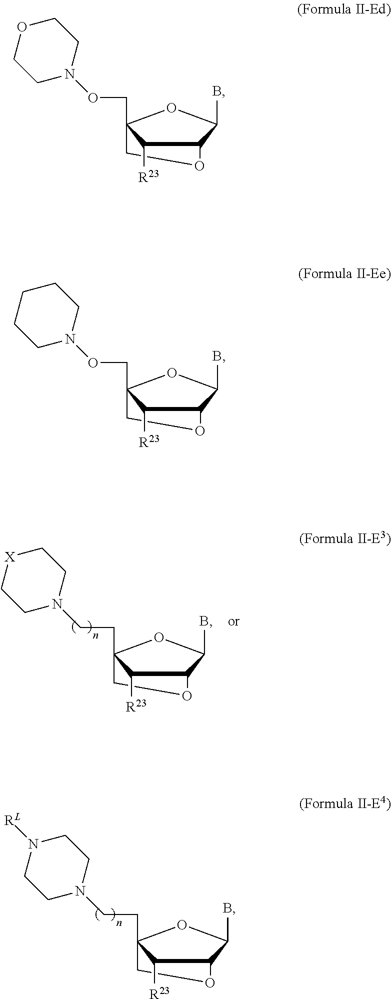
where n is 0 or an integer selected from 1 to 30 (e.g., n is 1, 2, 3, 4, 5, 6, 7, 8, 9, 10, 11, 12, 13, 14, 15, 16, 16, 17, 18, 19, 20, 21, 22, 23, 24, 25, 26, 27, 28, 29 or 30, such as n is 1, 2, 3, 4, 5 or 6, preferably n is 0 or 1); and
R^{L} is H, a ligand, a linker covalently bonded to one or more ligands, aliphatic and aromatic alkyl, alkylester, alkylamine, dimethylamino alkyl, alkylether, alkylthioether, heteroaromatic alkyl, allyl, vinyl, alkyl groups functionalized with disulfide, oxime, ketone, acetal, hemiacetal, cleavable peptides, or cleavable sugars, and
optionally, the compound is of Formula (II-Ef) or (II-Eg):

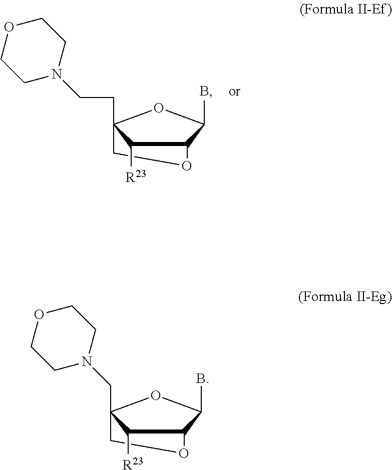

69. The oligonucleotide of any one of claims 65-68, wherein the oligonucleotide comprises from 3 to 50 nucleotides.

70. The oligonucleotide of any one of claims 65-69, wherein:
a. the oligonucleotide comprises at least one ribonucleotide;
b. the oligonucleotide comprises at least one 2′-deoxyribonucleotide;
c. the oligonucleotide comprises at least one nucleoside with a modified or non-natural nucleobase in addition to the nucleoside of Formula (II);
d. the oligonucleotide comprises at least one nucleoside with a modified ribose sugar in addition to the nucleoside of Formula (II);
e. the oligonucleotide comprises at least one nucleoside comprising a group other than H or OH at the 2′-position of the ribose sugar in addition to the nucleoside of Formula (II);
f. the oligonucleotide comprises at least one nucleoside with a 2′-F ribose in addition to the nucleoside of Formula (II);
g. the oligonucleotide comprises at least one nucleoside with a 2′-OMe ribose in addition to the nucleoside of Formula (II);
h. the oligonucleotide comprises at least one nucleoside comprising a moiety other than a ribose sugar in addition to the nucleoside of Formula (II);
i. the oligonucleotide comprises at least one modified internucleotide linkage;
j. the internucleotide linkage to the subsequent to the nucleoside of Formula (II) is a modified internucleotide linkage, optionally, the modified internucleotide linkage is a phosphorothioate linkage;
k. the oligonucleotide is attached to a solid support;
l. oligonucleotide comprises at least one ligand; and/or
m. the oligonucleotide comprises at least one hydroxyl, phosphate or amino protecting group.

71. A double-stranded nucleic acid comprising a first oligonucleotide strand and a second oligonucleotide strand substantially complementary to the first strand, wherein the first or second strand is an oligonucleotide of any one of claims 64-70, optionally, the first and second strand are independently 15 to 25 nucleotides in length.

72. The double-stranded nucleic acid claim 71, wherein double-stranded nucleic acid is capable of inducing RNA interference.

73. The double-stranded nucleic acid of claim 71 or 72, wherein one of the first stand and second strand is the oligonucleotide of any one of claims 64-70 and the other strand comprises on its 5′-end a vinylphosphonate group (VP) group (e.g., *═CH—X^{P}, X^{P} is a phosphate group and * is C5′), C₃₋₆ cycloalkylphosphonate (e.g., cyclopropylphosphonate), monophosphate ((HO)₂(O) P—O-5′), diphosphate ((HO)₂(O) P—O—P(HO)(O)—O-5′), triphosphate ((HO)₂(O) P—O—(HO)(O) P—O—P(HO)(O)—O-5′); monothiophosphate (phosphorothioate, (HO)2 (S) P—O-5′), monodithiophosphate (phosphorodithioate; (HO)(HS)(S) P—O-5′), phosphorothiolate ((HO)₂(O) P—S-5′); alpha-thiotriphosphate; beta-thiotriphosphate; gamma-thiotriphosphate; phosphoramidates ((HO)₂(O) P—NH-5′, (HO)(NH₂)(O) P—O-5′), alkylphosphonates [(R^{P})(OH)(O) P—O-5′, R^{P} is optionally substituted C₁₋₃₀ alkyl, e.g., methyl, ethyl, isopropyl, or propyl)], alkyletherphosphonates [(R^{P1})(OH)(O) P—O-5′, R^{P1} is alkoxyalkyl, e.g., methoxymethyl (CH₂OMe) or ethoxymethyl], (HO)₂(X) P—O[—(CH₂)ₐ—O—P(X)(OH)—O]_{b}-5′ or (HO)₂(X) P—O[—(CH₂)ₐ—P(X)(OH)—O]_{b}-5′ or (HO)₂(X) P—[—(CH₂)ₐ—O—P(X)(OH)—O]_{b}-5′, or optionally substituted alkyl, and dialkyl terminal phosphates and phosphate mimics (e.g., HO[—(CH₂)ₐ—O—P(X)(OH)—O]_{b}-5′, H₂N[—(CH₂)ₐ—O—P(X)(OH)—O]_{b}-5′, H[—(CH₂)ₐ—O—P(X)(OH)—O]_{b}-5′, Me₂N[—(CH₂)ₐ—O—P(X)(OH)—O]_{b}-5′, HO[—(CH₂)ₐ—P(X)(OH)—O]_{b}-5′, H₂N[—(CH₂)ₐ—P(X)(OH)—O]_{b}-5′, H[—(CH₂)ₐ—P(X)(OH)—O]_{b}-5′, Me₂N[—(CH₂)ₐ—P(X)(OH)—O]_{b}-5′, wherein X is O or S; and a and b are each independently 1-10, optionally, the strand comprises a vinylphosphonate group, e.g., an E-vinylphosphonate group.

74. The double-stranded nucleic acid of any one of claims 71-73, wherein the double-stranded nucleic acid comprises a sense strand and an antisense strand, and wherein the sense strand is the oligonucleotide of any one of claims 64-70.

75. The double-stranded nucleic acid of any one of claims 71-74, wherein one or both strands have a 1-5 nucleotide overhang on its respective 5′-end or 3′-end, optionally, only one strand has a 2 nucleotide overhang on its 5′-end or 3′-end, optionally, only one strand has a 2 nucleotide overhand on its 3′-end.

76. A pharmaceutical composition comprising an oligonucleotide of any one of claims 64-70 or dsRNA molecule of any one of claim 1-33 or 71-75, alone or in combination with a pharmaceutically acceptable carrier or excipient.

77. A gene silencing kit containing an oligonucleotide of any one of claims 64-70 or dsRNA molecule of any one of claim 1-33 or 71-75.

78. A method for silencing a target gene in a cell, the method comprising a step of introducing into the cell:
(i) a double-stranded RNA according to any one of claim 1-33 or 71-75, wherein the antisense strand comprises a nucleotide sequence substantially complementary to the target gene; or
(ii) an oligonucleotide according to any one of claims 64-70, wherein the oligonucleotide comprises a nucleotide sequence substantially complementary to the target gene.

79. A method of reducing the expression of a target gene in a subject, comprising administering to the subject either:
(i) a double-stranded RNA according to any one of claim 1-33 or 71-75, wherein the antisense strand comprises a nucleotide sequence substantially complementary to the target gene; or
(ii) an oligonucleotide according to any one of claims 64-70, wherein the oligonucleotide comprises a nucleotide sequence substantially complementary to a target gene.

80. The method of claim 79, wherein said administering is subcutaneous or intravenous administration.
